(19) United States
(12) Patent Application Publication
(10) Pub. No.: US 2026/0109988 A1
(43) Pub. Date: Apr. 23, 2026

(54) COMPOSITIONS AND METHODS TO MODULATE TUMOR MICROENVIRONMENT ASSOCIATED LONG NONCODING RNAS

(71) Applicant: HAYA Therapeutics SA, Epalinges (CH)

(72) Inventors: Samir OUNZAIN, Lausanne (CH); Emely MöLLER TORDENMALM, Mont-sur-Lausanne (CH); Daniel Roman BLESSING, Saint-Sulpice (CH)

(21) Appl. No.: 19/424,522

(22) Filed: Dec. 18, 2025

Related U.S. Application Data
(63) Continuation of application No. PCT/IB2024/000333, filed on Jun. 21, 2024.
(60) Provisional application No. 63/523,001, filed on Jun. 23, 2023.
(60) Provisional application No. 63/587,332, filed on Oct. 2, 2023.
(60) Provisional application No. 63/651,270, filed on May 23, 2024.

(51) Int. Cl. C12N 15/113; A61K 31/712; A61K 31/7125; A61K 45/06; C12Q 1/6886

(52) U.S. Cl. CPC C12N 15/1138; A61K 31/712; A61K 31/7125; A61K 45/06; C12N 15/1137; C12Q 1/6886; C12N 2310/11; C12N 2310/14; C12N 2310/315; C12N 2310/321; C12N 2310/3231; C12N 2310/341; C12Q 2600/106; C12Q 2600/118; C12Y 207/12001

(57) ABSTRACT

Disclosed herein are modulators of a long non-coding RNA (lncRNA), wherein an expression of the lncRNA is associated with an activation of a cancer-associated fibroblast (CAF). Further provided herein are methods of inhibiting a growth of a solid tumor or facilitating access to a solid tumor in a subject in need thereof.

### CROSS REFERENCE

This application is continuation of PCT Application No. PCT/IB2024/000333, filed Jun. 21, 2024, which claims the benefit of U.S. Provisional Application No. 63/523,001, filed Jun. 23, 2023, U.S. Provisional Application No. 63/587,332, filed Oct. 2, 2023, and U.S. Provisional Application No. 63/651,270, filed May 23, 2024, each of which are incorporated herein by reference in their entireties.

### SEQUENCE LISTING

The instant application contains a Sequence Listing which has been submitted electronically in XML format and is hereby incorporated by reference in its entirety. Said XML copy, created on Sep. 3, 2024, is named 60448-705_601_SL.xml and is 2,090,611 bytes in size.

### BACKGROUND

Only a small portion of mammalian genome is transcribed to protein-coding mRNAs, with the rest is transcribed to non-coding RNAs. Among them, long non-coding RNAs (lncRNA) are a type of RNA with usually more than 200 nucleotides that are not translated into protein. LncRNAs have been shown to regulate gene expression networks at different levels via various mechanisms (see, e.g., Yao et al., Nature Cell Biology, 21, pages 542-551, 2019).

Cancer-associated fibroblasts (CAFs) are a group of activated fibroblasts. Fibroblasts may change to CAFs depending on their microenvironment. Furthermore, CAFs are highly heterogeneous and display plasticity in the tumor microenvironment. CAFs are involved in well characterized pro-tumorigenic functions, such as participating in the growth and invasion of tumor cells, that contribute to the development of various cancers. They can secrete various factors to regulate tumor development, metastasis, and therapeutic resistance.

Due to their involvement in tumor-promoting functions, CAFs may be important targets for cancer therapy; however, there are no fundamental CAF-targeting therapies. Therefore, there exist a need to develop CAF-targeting therapies that directly or indirectly modulate the development of CAF and/or activity of CAF in the tumor microenvironment. Such CAF-targeting therapies may be beneficial to prevent, alleviate, and treat solid cancers.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY OF THE DISCLOSURE

The present disclosure provides the identification of exemplary long non-coding RNAs (lncRNAs) that are upregulated in cancer-associated fibroblast (CAF) and provides compositions and methods of inhibiting a growth of a solid tumor or facilitating access to a solid tumor in a subject in need thereof by modulating (e.g., activating, suppressing, or supplementing) the expression of activity of the lncRNAs.

Provided herein is a modulator of a long non-coding RNA (lncRNA), wherein an expression or activity of the lncRNA is associated with an activation of a cancer-associated fibroblast (CAF).

In some instances, the expression or activity of the lncRNA is upregulated in the CAF compared to fibroblast not associated with cancer.

In some instances, the expression or activity of the lncRNA is not upregulated in a cell that is not associated with cancer.

In some instances, the CAF is associated with collagen-fibril organization, extracellular matrix (ECM) organization, and/or tissue development in tumor microenvironment. In some instances, the CAF exhibits a higher expression of an ECM-modulatory gene when compared to a fibroblast that is not associated with cancer. In some instances, the ECM-modulatory gene is selected from the group consisting of C1QTNF3, COL5A2, ITGA11, PDPN, POSTN, ACTA2, ACTN1, ADAM12, ADAMTS12, AEBP1, ALDH18A1, ANTXR1, ARF4, ARL4C, BACE2, BASP1, BGN, BHLHE40, BMP1, BST2, C11orf24, C1orf198, C1QTNF6, CADM1, CALD1, CALU, CCND1, CD276, CDC42EP3, CERCAM, CHN1, CHPF, CKAP4, CLEC11A, CLIC4, CNN2, COL10A1, COL11A1, COL12A1, COL1A1, COL1A2, COL5A1, COL8A1, COLGALT1, CREB3L1, CSRP2, CTHRC1, CTSB, CTSK, CTSZ, CXCL2, CXCL3, DAP, DIO2, DPYSL3, DUSP10, EDIL3, EDNRA, EFEMP2, EGFL6, ERN1, FAP, FBXO32, FKBP10, FN1, FSCN1, FZD1, GAPDH, GEM, GGT5, GJA1, GLT8D2, GOLM1, GPX7, GPX8, GREM1, HAPLN3, HCFC1R1, HES4, HLA-B, HLA-C, HS3ST3A1, ID1, ID4, IER3, IFI27, IFI6, IL32, INHBA, ITGB5, ITPRIP, KDELR2, KDELR3, KIAA1217, KIF26B, KLF6, LAMP5, LEF1, LMCD1, LMO7, LOXL2, LRRC15, LUM, MAGED1, MARCKSL1, MARVELD1, MDK, MICAL2, MIF, MMP11, MMP14, MMP19, MMP2, MSRB3, MXRA5, MYH9, MYL9, NEK6, NR4A2, NREP, NRP2, NTM, NXN, OLFML2B, P3H1, P3H4, P4HA3, P4HB, PALLD, PARVA, PDGFC, PDLIM7, PEA15, PERP, PKM, PLOD1, PLOD2, PMAIP1, PMEPA1, PODNL1, POSTN, PRDX4, PRSS23, PTGER3, PTK7, PYCR1, RAB31, RAI14, RBM3, RCAN2, RCN1, RCN3, RGCC, RGS3, RIN2, RNF144A, ROR2, RUNX2, SCARF2, SDC1, SEC13, SERPINHI, SFRP2, SHISA5, SLC16A3, SLC38A5, SLC39A14, SMCO4, SMIM3, SMYD3, SNAI2, SPARC, SPATS2L, SPHK1, SPON1, SSR3, STK17B, SUGCT, SULF1, SULF2, SYTL2, TAGLN, TENM3, TGFB1I1, TGFB1, THBS2, THY1, TMEM119, TMEM263, TMEM45A, TNFAIP3, TOM1, TPM1, TPM4, TPST2, TSPO, TUBA1C, TUSC3, UBTD1, UNC5B, VCAN, VGLL4, and VOPPl.

In some instances, one or more enhancers are within a genomic locus encoding the lncRNA, wherein the one or more enhancers are associated with the CAF. In some instances, the one or more enhancers are marked by H3K27ac.

In some instances, one or more protein-coding genes are at most 100, 200, 300, 400, or 500 nucleotides upstream or downstream of or within the genomic locus encoding the lncRNA, wherein the one or more protein-coding genes are associated with the CAF. In some instances, the one or more protein-coding genes are associated with a pro-tumorigenic or fibrosis development function of the CAF. In some instances, the one or more protein-coding genes are selected from the group consisting of LRRC15, COL12A1, DYRK2, FN1, and CAPN9.

In some instances, the lncRNA comprises XLOC_055514, XLOC_055515, XLOC_069921, XLOC_005184, ENSG00000203585, SHARED_00113753, as listed in Table 10, any one of lncRNAs listed in Table 12, or a fragment thereof. In some instances, the lncRNA comprises XLOC_055514, XLOC_055515, XLOC_069921, XLOC_005184, ENSG00000203585, SHARED_00113753, ENSG00000288903, ENSG00000230838 (LINC01614), ENSG00000244137 as listed in Table 10, or a fragment thereof.

In some instances, the modulator comprises an endonuclease complex guided by a nucleic acid, wherein the nucleic acid targets the lncRNA or a genomic locus thereof. In some instances, the modulator is a CRISPR-directed DNA editing complex. In some instances, the modulator is an ASO-directed RNA editing complex.

In some instances, the modulator comprises a nucleic acid molecule that hybridizes to the lncRNA. In some instances, the nucleic acid molecule is a small interfering RNA (siRNA), a microRNA (miRNA), an inhibitory double stranded RNA (dsRNA), a small or short hairpin RNA (shRNA), an antisense oligonucleotide (ASO), a piwi-interacting RNA (piRNA), a heterogeneous nuclear RNA (hnRNA), a small nuclear RNA (snRNA), or an enzymatically-prepared siRNA (esiRNA) or the precursors thereof.

In some instances, the nucleic acid molecule is an ASO. In some instances, the ASO is a gapmer or a mixmer. In some instances, the ASO is about 13-30 nucleotides long. In some instances, the ASO is about 14-18 nucleotides long.

In some instances, the nucleic acid molecule comprises at least 10, 11, 12, or 13 consecutive nucleotides with no more than 1, 2, or 3 mismatches from one of SEQ ID NOs: 1-40. In some instances, the nucleic acid molecule comprises a nucleic acid sequence that is at least 80%, at least 85%, at least 90%, at least 95% identical to a sequence selected from one of SEQ ID NOs: 1-40.

In some instances, the ASO is a gapmer comprising a central region of consecutive DNA nucleotides flanked by a 5′-wing region and 3′-wing region, wherein at least one of 5′-wing region and 3′-wing region comprises a nucleic acid analogue, wherein the nucleic acid analogue comprises one or more ribose modifications, one or more backbone modifications, one or more nucleobase modifications, or a combination thereof.

In some instances, the nucleic acid analogue comprises a 2′-methoxyethyl (2′-MOE) RNA.

In some instances, the nucleic acid analogue comprises a locked nucleic acid (LNA). In some instances, the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA. In some instances, the LNA comprises a beta-D-oxy LNA. In some instances, the 5′-wing region comprises at least two LNAs. In some instances, the 5′-wing region comprises three consecutive LNAs. In some instances, the 3′-wing region comprises at least one LNA. In some instances, the 3′-wing region comprises two consecutive LNAs.

In some instances, the nucleic acid molecule comprises one or more phosphorothioate internucleotide linkages. In some instances, each internucleotide linkage in the nucleic acid molecule is a phosphorothioate backbone.

Provided herein is a modulator comprising an antisense oligonucleotide (ASO), wherein the ASO comprises at least 10, 11, 12, or 13 consecutive nucleotides with no more than 1, 2, or 3 mismatches from SEQ ID NO: 2, 4, 5, 9-11, 15, 16, 18, 19, 24-26, 34 or 38-40.

In some instances, the ASO comprises a nucleic acid sequence of 80%, at least 85%, at least 90%, at least 95% identical to SEQ ID NO: 2, 4, 5, 9-11, 15, 16, 18, 19, 24-26, 34 or 38-40.

Further provided herein is a pharmaceutical composition comprising the modulator described herein and a pharmaceutically acceptable salt or derivative thereof.

Further provided herein is a kit comprising the modulator described herein or the pharmaceutical composition described herein.

Further provided herein is a method of inhibiting a growth of a solid tumor or facilitating access to a solid tumor in a subject in need thereof, the method comprising administering to the subject an effective amount of the modulator described herein, or the pharmaceutical composition described herein.

In some instances, the modulator described herein, or the pharmaceutical composition described herein reduces expression or activity of genes involved in ECM structure organization and/or regulation of GTPase activity. In some instances, the modulator described herein, or the pharmaceutical composition described herein reduces expression or activity of genes involved in regulation of tumor innervation in a tumor microenvironment. In some instances, the modulator described herein, or the pharmaceutical composition described herein reduces an expression or activity of one or more genes shown in Table 1, Table 13, or Table 14. In some instances, the modulator described herein, or the pharmaceutical composition described herein reduces an expression or activity of an ECM-modulatory gene in the CAF. In some instances, the ECM-modulatory gene or the genes involved in ECM structure organization and/or regulation of GTPase activity and/or regulation of tumor innervation in a tumor microenvironment is selected from the group consisting of LRRC15, MMP11, COL11A1, C1QTNF3, CTHRC1, COL12A1, COL10A1, COL5A2, THBS2, AEBP1, ITGA11, PDPN, and FAP.

In some instances, the modulator described herein is encoded by a transgene in an expression vector.

In some instances, the modulator described herein is encapsulated in a liposome or coupled with a nanoparticle.

In some instances, the modulator described herein is administered in combination with an anti-tumor drug.

Further provided herein is a method of diagnosing or monitoring a cancer in a subject, the method comprising: (a) obtaining a sample from the subject; (b) detecting an expression and/or an activity of a biomarker from the sample, wherein the biomarker comprises an lncRNA comprising a nucleic acid sequence listed in Table 11 or a fragment thereof, or transcribed from a genomic region or a subset thereof listed in Table 10 or Table 12; and (c) diagnosing or monitoring the cancer prognosis based on the expression and/or the activity of the biomarker. In some instances, the biomarker further comprises INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1, or a gene from Table 1, Table 13, or Table 14. In some instances, the method further comprises (d) administering the subject the modulator disclosed herein, or the pharmaceutical composition disclosed herein. In some instances, the cancer is a solid tumor. In some instances, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma.

Further provided herein is a method of predicting severity and progression of a cancer in a subject, the method comprising: (a) obtaining a sample from the subject; (b) detecting an expression and/or an activity of a biomarker from the sample, wherein the biomarker comprises an lncRNA comprising a nucleic acid sequence listed in Table 11 or a fragment thereof, or transcribed from a genomic region or a subset thereof listed in Table 10 or Table 12; and (c) diagnosing the subject to have a more severe or a progression of the cancer if the expression and/or the activity of the biomarker is at least 50%, at least 60%, at least 70%, at least 80%, or at least 90% higher when compared to a control. In some instances, the biomarker further comprises INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1, or a gene from Table 1, Table 13, or Table 14. In some instances, the method further comprises (d) administering the subject the modulator disclosed herein, or the pharmaceutical composition disclosed herein. In some instances, the cancer is a solid tumor. In some instances, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma.

Further provided herein is a method of monitoring an efficacy or therapeutic resistance of a therapy treating a cancer in a subject, the method comprising: (a) obtaining a sample from the subject; (b) detecting an expression and/or an activity of a biomarker from the sample, wherein the biomarker comprises an lncRNA comprising a nucleic acid sequence listed in Table 11 or a fragment thereof, or transcribed from a genomic region or a subset thereof listed in Table 10 or Table 12; and (c) concluding the therapy treating the cancer is effective or is less likely to develop therapeutic resistance if the expression and/or the activity of the biomarker is at least 50%, at least 60%, at least 70%, at least 80%, at least 90% reduced by the therapy. In some instances, the biomarker further comprises INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1, or a gene from Table 1, Table 13, or Table 14. In some instances, the method further comprises (d) administering the subject the modulator disclosed herein, or the pharmaceutical composition disclosed herein. In some instances, the cancer is a solid tumor. In some instances, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative instances, in which the principles of the disclosure are utilized, and the accompanying drawings (also “Figure” and “FIG.” herein), of which:

FIGS. 1A-1F illustrate cluster analysis of target lncRNA candidates in primary human Head & Neck Squamous Cell Carcinoma (HNSCC) tumor samples. FIG. 1A shows expression of candidate target lncRNAs in HNSCC tumor fibroblast (FB) sub-clusters. Stromal Enhancer Associated lncRNAs (SEALs; SEAL1-SEAL7) are expressed in FBs, whereof SEAL1, SEAL3, SEAL5 and SEAL6 are expressed in fibroblast cluster 2, representing myofibroblast Cancer Associated Fibroblast (myCAF). FIG. 1B shows differential expression analysis of SEALs in The Cancer Genome Atlas (TCGA)-HNSCC tumor tissue versus normal tissue, and in tumor tissue fibroblasts versus normal tissue fibroblasts. FIG. 1C shows expression of SEALs across normal human in vitro and in vivo tissues and cells with Encyclopedia of DNA Elements (ENCODE)/Genotype-Tissue Expression (GTEx) database. FIG. 1D is a heat map showing expression of differentially upregulated candidate lncRNAs in the fibroblast cluster 2, representing myofibroblast Cancer Associated Fibroblast (myCAF). FIG. 1Ei-1Eiv shows dot graphs of expression of differentially upregulated candidate lncRNAs across normal human in vitro and in vivo tissues and cells with Encyclopedia of DNA Elements (ENCODE)/Genotype-Tissue Expression (GTEx) databases. FIG. 1F shows dot graphs of expression of lncRNAs associated with the myCAF signature across normal human in vitro and in vivo tissues and cells with Encyclopedia of DNA Elements (ENCODE)/Genotype-Tissue Expression (GTEx) databases.

FIGS. 2A-2O illustrate the generation and characterization of an in vitro induced Cancer Associated Fibroblast (CAF). FIG. 2A shows the experimental scheme of generating the in vitro induced CAF by treating Human Dermal Fibroblasts (HDFs) with TGFβ or TGFβ+ starvation (COMB). FIG. 2B shows microscopic images of HDFs upon TGFβ or COMB induction versus no induction (control), at 12 hours, 24 hours and 48 hours. FIG. 2C shows upregulation of myofibroblast CAF (myCAF) markers upon TGFβ or COMB induction by RNA-seq analysis. FIG. 2D shows expression of myofibroblast CAF markers upon TGFβ induction by qPCR analysis. FIG. 2E shows expression of myofibroblast CAF markers upon COMB induction by qPCR analysis. FIG. 2F shows expression of SEAL1 in control HDFs, and induced CAFs by COMB or TGFβ by RNA-seq analysis. FIG. 2G shows expression of SEAL2 in control HDFs, and induced CAFs by COMB or TGFβ by RNA-seq analysis. FIG. 2H shows expression of SEAL3 in control HDFs, and induced CAFs by COMB or TGFβ by RNA-seq analysis. FIG. 2I shows expression of SEAL4 in control HDFs, and induced CAFs by COMB or TGFβ by RNA-seq analysis. FIG. 2J shows expression of SEAL5 in control HDFs, and induced CAFs by COMB or TGFβ by RNA-seq analysis. FIG. 2K shows expression of SEAL6 in control HDFs, and induced CAFs by COMB or TGFβ by RNA-seq analysis. FIG. 2L shows expression of SEAL7 in control HDFs, and induced CAFs by COMB or TGFβ by RNA-seq analysis. FIG. 2M shows a heat map of differentially upregulated myofibroblast CAF markers upon TGFβ induction by RNA-seq analysis. FIG. 2N shows upregulation of myofibroblast CAF markers upon TGFβ induction by qPCR analysis. FIG. 2O shows SingScore analysis of a defined myCAF signature upon TGFβ induction by RNA-seq analysis.

FIGS. 3A-3F illustrate characterization of SEAL1 and antisense oligonucleotides (ASOs) targeting SEAL1. FIG. 3A shows genomic locus encoding SEAL1 transcript and LRRC15 mRNA, and SEAL1 upregulation in the TGFβ induced CAF. FIG. 3B shows genomic location where SEAL1-specific tagged primers and LRRC15-specific tagged primers are hybridized. FIG. 3C shows qPCR amplification using LRRC15-specific tagged primers and SEAL1-specific tagged primers with control versus TGFβ induced CAF. FIG. 3D shows qPCR validation of SEAL1 expression in TGFβ induction CAF model versus control. FIG. 3E shows expression of SEAL1, myCAF markers, and LRRC15 markers in TGFβ induced CAF versus control. FIG. 3F shows genomic locations targeted by 7 different designs of SEAL1 ASOs.

FIGS. 4A-4I illustrate qPCR analysis for expression of SEAL1 and myCAF markers upon transfecting TGFβ induced CAFs with SEAL1 ASOs or scramble ASO control (Scrl).

FIGS. 4J-4K illustrate analysis of LRRC15 protein upon transfecting TGFβ induced CAFs with SEAL1 ASO, scramble ASO control (Scrl) or LRRC15 ASO. FIG. 4J shows western blot of LRRC15 protein levels across different conditions. FIG. 4K shows the quantification of FIG. 4J.

FIG. 5 shows microscopic images of TGFβ induced CAFs transfected with SEAL1 ASO (SEAL1_G2, SEAL1_G4, or SEAL1_G5) or scramble control ASO (scrl ASO).

FIGS. 6A-6I illustrate qPCR analysis for expression of SEAL1 and myCAF markers upon transfecting TGFβ induced CAFs with SEAL1_G4 ASO, SEAL1_G5 ASO, or Scrl.

FIG. 6J shows RNA-seq analysis of SEAL1 transcript upon transfecting TGFβ induced CAFs with SEAL1_G4 ASO, SEAL1_G5 ASO, or Scrl. FIG. 6K shows SingScore of myCAF gene signature upon transfecting TGFβ induced CAFs with SEAL1_G4 ASO, SEAL1_G5 ASO, or Scrl.

FIG. 7A shows genomic locations targeted by 7 different designs of LRRC15 ASOs. FIGS. 7B-7J illustrate qPCR analysis for expression of SEAL1 and myCAF markers upon transfecting TGFβ induced CAFs with LRRC15 ASOs. FIG. 7K-7Q illustrate qPCR analysis for expression of myCAF markers upon transfecting TGFβ induced CAFs with SEAL1_G4 ASO, SEAL1_G5 ASO, LRRC15_G1 ASO, LRRC15_G3 ASO, or Scrl.

FIG. 8 shows microscopic images of TGFβ induced CAFs transfected with LRRC15 ASO (LRRC15_G1, LRRC15_G2, or LRRC15_G3) or scramble control ASO (scrl ASO).

FIG. 9A-9S illustrates the analysis of SEAL1 and LRRC15 in HNSCC FB and TGFβ induced CAFs. FIG. 9A shows RNA-seq analysis of LRRC15 mRNA upon transfecting TGFβ induced CAFs with LRRC15_GI ASO, LRRC15_G3 ASO, or Scrl. FIG. 9B shows SingScore analysis of a defined myCAF signature upon transfecting TGFβ induced CAFs with SEAL1_G4 ASO, SEAL1_G5 ASO, LRRC15_G1 ASO, LRRC15_G3 ASO, or Scrl. FIG. 9C shows single nuclei RNA-seq (snRNA-seq) analysis of SEAL1 and LRRC15 transcripts, as well as SingScore analysis of a defined myCAF signature and pseudobulk quantification of the myCAF signature upon transfecting TGFβ induced CAFs with SEAL1_G4 ASO, LRRC15_G3 ASO, or Scrl. FIG. 9D illustrates the analysis of SEAL1 and mRNA LRRC15 expression in HNSCC FBs of cancer progression stages from HNSCC scRNA-seq data. FIG. 9E shows RNA-seq analysis of the SEAL1 transcript expression upon transfecting TGFβ induced CAFs with the SEAL1_G4 ASO at different doses (0, 0.5, 1, 5, 10 and 25 nM). FIG. 10F shows RNA-seq analysis of the LRRC15 (top, left), FAP (top, right), CTHRC1 (bottom, left) and POSTN (bottom, right) transcripts expression upon transfecting TGFβ induced CAFs with the SEAL1_G4 ASO at different doses (0, 0.5, 1, 5, 10 and 25 nM). FIG. 9G shows Singscore analysis of an in-house defined myCAF signature (Table 13) upon transfecting TGFβ induced CAFs with the SEAL1_G4 ASO at different doses (0, 0.5, 1, 5, 10 and 25 nM). FIG. 10H shows RNA-seq analysis of the LRRC15 transcript expression upon transfecting TGFβ induced CAFs with the LRRC15_G3 ASO at different doses (0, 0.5, 1, 5, 10 and 25 nM). FIG. 9I shows RNA-seq analysis of the SEAL1 (top, left), FAP (top, right), CTHRC1 (bottom, left) and POSTN (bottom, right) transcripts expression upon transfecting TGFβ induced CAFs with the LRRC15_G3 ASO at different doses (0, 0.5, 1, 5, 10 and 25 nM). FIG. 9J shows Singscore analysis of an in-house defined myCAF signature (Table 13) upon transfecting TGFβ induced CAFs with the LRRC15_G3 ASO at different doses (0, 0.5, 1, 5, 10 and 25 nM). FIG. 9K shows identification of the SEAL1 Target Engagement panel (SEAL1 TEP) genes. Left: RNA-seq dose response cluster analysis upon transfecting TGFβ induced CAFs with the SEAL1_G4 ASO at different doses (0, 0.5, 1, 5, 10 and 25 nM). Right: Singscore analysis of the most responsive gene cluster (n=526), called SEAL1 TEP (Table 14), upon transfecting TGFβ induced CAFs with the SEAL1_G4 ASO at different doses (0, 0.5, 1, 5, 10 and 25 nM). FIG. 9L shows the fibroblast sub-clusters (top left), the SEAL1 expression in primary cancer fibroblasts (top middle) and Singscore analysis of the SEAL1 TEP in all fibroblasts (top right), of a HNSCC scRNA-seq dataset. Bottom figure panel shows Singscore analysis of a SEAL1 co-expression module (hSEAL1 module, n=177), defined from HNSCC primary cells unbiased co-expression network analysis, in HNSCC normal tissue FBs (left) vs primary cancer FBs (right, also shown in the right subfigure of FIG. 9M). FIG. 9M (left) shows Singscore analysis of the SEAL1 module (n=177) upon transfection of TGFβ induced CAFs with control Scrl, SEAL1_G4 and LRRC15_G3 ASOs. FIG. 9M (right) also shows Singscore analysis of the SEAL1 module (n=177) in HNSCC primary cancer FBs. FIG. 9N shows RNA-seq analysis of the LRRC15 transcript expression upon transfecting non-TGFβ induced (control) dCAS9-iHDF cells with non-targeting sgRNA and non-targeting Scrl ASO, or upon transfecting TGFβ-induced dCAS9-iHDF cells with either non-targeting sgRNA plus Scrl ASO (TGFβ control) or with LRRC15-targeting sgRNA plus Scrl ASO (LRRC15 depletion only). FIG. 9O shows RNA-seq analysis of the COL11A1, CTHRC1, COL5A2 and ITGA11 transcripts expression upon transfecting non-TGFβ induced (control) dCAS9-iHDF cells with non-targeting sgRNA and non-targeting Scrl ASO, or upon transfecting TGFβ-induced dCAS9-iHDF cells with either non-targeting sgRNA plus Scrl ASO (TGFβ control), with LRRC15-targeting sgRNA plus Scrl ASO (LRRC15 depletion only), with non-targeting sgRNA plus SEAL1_G4 ASO (SEAL1 depletion only) or with LRRC15-targeting sgRNA plus SEAL1_G4 ASO (depletion of both LRRC15 and SEAL1). FIG. 9P shows RNA-seq Singscore analysis of the myCAF signature upon transfecting non-TGFβ induced (control) dCAS9-iHDF cells with non-targeting sgRNA and non-targeting Scrl ASO, or upon transfecting TGFβ-induced dCAS9-iHDF cells with either non-targeting sgRNA plus Scrl ASO (TGFβ control), with LRRC15-targeting sgRNA plus Scrl ASO (LRRC15 depletion only), with non-targeting sgRNA plus SEAL1_G4 ASO (SEAL1 depletion only) or with LRRC15-targeting sgRNA plus SEAL1_G4 ASO (depletion of both LRRC15 and SEAL1). FIG. 9Q shows western blot analysis of the LRRC15 protein in control dCAS9-iHDF cells, or in TGFβ-induced dCAS9-iHDF cells transfected with non-targeting Scrl ASO, with LRRC15-targeting sgRNA plus Scrl ASO (LRRC15 depletion only, LRRC15 CRISPRi+Scrl), with non-targeting sgRNA plus SEAL1_G4 ASO (SEAL1 depletion only, hSEA1_4) or with LRRC15-targeting sgRNA plus SEAL1_G4 ASO (depletion of both LRRC15 and SEAL1, LRRC15 CRISPRi+hSEA1_4). FIG. 9R shows RNA-seq analysis of the SEAL1 transcript expression or LRRC15+ myofibroblast marker signature score upon transfecting non-TGFβ induced (control) dCAS9-iHDF cells with non-targeting sgRNA and non-targeting Scrl ASO, or upon transfecting TGFβ-induced dCAS9-iHDF cells with either non-targeting sgRNA plus Scrl ASO (TGFβ control), with LRRC15-targeting sgRNA plus Scrl ASO (LRRC15 depletion only), with non-targeting sgRNA plus SEAL1_G4 ASO (SEAL1 depletion only) or with LRRC15-targeting sgRNA plus SEAL1_G4 ASO (depletion of both LRRC15 and SEAL1). FIG. 9S shows RNA-seq analysis of the TNC (left), FAP (top, right), COL5A1 (middle) and LTBP2 (right) transcripts expression upon transfecting TGFβ induced CAFs with the SEAL1_G4 ASO at different doses.

FIGS. 10A-10F illustrate characterization of SEAL2 and ASOs targeting SEAL2. FIG. 10A shows genomic locus encoding SEAL2 transcript and COL12A1 mRNA, and SEAL2 upregulation in the COMB induced CAF. FIG. 10B shows genomic locus encoding CAF super enhancer (SE) region and COL12A1 mRNA and its upregulation in the COMB induced CAF. FIG. 10C shows genomic location where SEAL2-specific primers are hybridized. FIG. 10D shows qPCR validation of SEAL2 expression in COMB induced CAF versus control. FIG. 10E shows expression of SEAL2 and myCAF markers in COMB induced CAF versus control. FIG. 10F shows genomic locations targeted by 8 different designs of SEAL2 ASOs.

FIGS. 11A-11I illustrate qPCR analysis for expression of SEAL2 and myCAF markers upon transfecting COMB induced CAFs with SEAL2 ASOs or Scrl.

FIG. 12 shows microscopic images of COMB induced CAFs transfected with SEAL2 ASO (SEAL2_G4, SEAL2_G5, SEAL2_G7, or SEAL2_G8) or scramble control ASO (scrl ASO).

FIGS. 13A-13I illustrate qPCR analysis for expression of SEAL2 and myCAF markers upon transfecting COMB induced CAFs with SEAL2_G4 ASO, SEAL2_G5 ASO, SEAL2_G8, or Scrl. FIG. 13J shows RNA-seq analysis of SEAL2 transcript upon transfecting COMB induced CAFs with SEAL2_G4 ASO, SEAL2_G8 ASO, or Scrl. FIG. 13K shows SingScore of myCAF gene signature upon transfecting COMB induced CAFs with SEAL2_G4 ASO, SEAL2_G8 ASO, or Scrl.

FIGS. 14A-14R illustrate SEAL3 identification and functions. FIGS. 14A-14C illustrate characterization of SEAL3 and ASOs targeting SEAL3. FIG. 14A shows genomic locus encoding SEAL3 transcript, and SEAL3 upregulation in the TGFβ and COMB induced CAF. FIG. 14B shows genomic location where SEAL3-specific primers are hybridized, and genomic locations targeted by 8 different designs of SEAL3 ASOs. FIG. 14C shows qPCR validation of SEAL3 expression in COMB induced CAF versus control. FIGS. 14D-14J illustrate qPCR analysis for expression of myCAF markers upon transfecting COMB induced CAFs with SEAL3_G1 ASO, SEAL3_G2 ASO, SEAL3_G3 ASO, SEAL3_G4 ASO, SEAL3_G5 ASO, SEAL3_G6 ASO, SEAL3_G7 ASO, SEAL3_G8 ASO or Scrl. FIG. 14K shows microscopic images of COMB induced CAFs transfected with SEAL3 ASO (SEAL3_G5, SEAL3_G6 or SEAL3_G7) or scramble control ASO (scrl ASO). FIGS. 14L-14R illustrate qPCR analysis for expression of myCAF markers upon transfecting COMB induced CAFs with SEAL3_G6 ASO, SEAL3_G7 ASO, or Scrl.

FIGS. 15A-15D illustrate SEAL4 identification and functions. FIGS. 15A-15D illustrate characterization of SEAL4 and ASOs targeting SEAL4. FIG. 15A shows genomic locus encoding SEAL4 transcript and DYRK2 mRNA, and SEAL4 upregulation in the TGFβ and COMB induced CAF. FIG. 15B shows genomic location where SEAL4-specific tagged primers are hybridized. FIG. 15C shows qPCR validation of SEAL4 expression using specific primers, specifically primer pairs 5 and 6 (PP5 and PP6). FIG. 15D shows genomic locations targeted by 6 different designs of SEAL4 ASOs.

FIGS. 15E-15J illustrate qPCR analysis for expression of SEAL4 and myCAF markers upon transfecting COMB induced CAFs with SEAL4_G1 ASO, SEAL4_G3 ASO, SEAL4_753_G1 ASO, SEAL4_753_G4 ASO, SEAL4_753_G5 ASO, SEAL4_753_G6 ASO, SEAL4_753_G7 ASO, or Scrl.

FIG. 15K shows microscopic images of COMB induced CAFs transfected with SEAL4 ASO (SEAL4_753_G1, SEAL4_753_G5, SEAL4_753_G6 or SEAL4_753_G7) or scramble control ASO (scrl ASO).

FIGS. 15L-15Q illustrate qPCR analysis for expression of SEAL4 and myCAF markers upon transfecting COMB induced CAFs with SEAL4_753_G6 ASO, SEAL4_753_G7 ASO, or Scrl.

FIGS. 16A-16C illustrate characterization of SEAL9 and ASOs targeting SEAL9. FIG. 16A shows genomic locus encoding SEAL9 transcript. FIG. 16B shows genomic location where SEAL9-specific primers are hybridized and qPCR validation of SEAL9 upregulation in the TGFβ induced CAFs vs CTRL. FIG. 16C shows genomic locations targeted by 3 different SEAL9 ASOs.

FIGS. 17A-17H illustrate qPCR analysis for expression of SEAL9 and myCAF markers upon transfecting TGFβ induced CAFs with SEAL9_G1 ASO, SEAL9_G2 ASO, SEAL9_G3 ASO, or Scrl.

FIG. 18 shows microscopic images of TGFβ induced CAFs transfected with SEAL9 ASO (SEAL9_G1, SEAL9_G2, SEAL9_G3) or scramble control ASO (scrl ASO).

FIG. 19 shows analysis of SEAL2, SEAL3 and SEAL4 lncRNA transcripts expression in HNSCC FBs of cancer progression stages from human patient HNSCC scRNA-seq data.

FIGS. 20A-20I illustrate the mouse Seal1 identification and functions, and analysis of ASOs designed to target mouse Seal1 (mSeal1_1-mSeal1_6) or mouse Lrrc15 (mLrrc15_1-mLrrc15_3). FIG. 20A shows the mouse genomic locus encoding mSeal1 transcript and mLrrc15 mRNA, the expression of mSeal1 and mLrrc15 in immortalized mouse dermal fibroblasts (iMDFs) and TGFβ induced iMDFs by RNA-seq data, and location of >90%, >70% or >50% conserved regions between human SEAL1 and mouse mSeal1. FIG. 20B shows a drawing of the iMDF in vitro model (left) and the upregulation, as compared to control (CTRL), of mSeal1 and mLrrc15 in TGFβ induced iMDFs by RNA-seq analysis (right). FIG. 20C shows the mouse genomic locus encoding mSeal1 transcript and mLrrc15 mRNA, and the location of exemplary ASOs designed to target mSeal1 (mSeal1_1-mSeal1_6) or mLrrc15 (mLrrc15_1-mLrrc15_3).

FIG. 20D shows images of iMDFs (top), and iMDFs 48h after TGFβ induction and transfection with mSeal1_G5, mSeal1_G6 or mLrrc15_G3 ASOs (middle and bottom). FIG. 20E shows a diagram of the iMDF in vitro model (left) and RNA-seq analysis of the mSeal1 or mLrrc15 transcripts expression upon transfecting TGFβ induced iMDFs with the mSeal1_G6 (mSeal1_6), mLrrc15_G3 (mLrrc15_3) or control Scrl ASOs. FIG. 20F shows Singscore analysis of the mouse orthologous myCAF signature (Table 13) (left) or the mouse orthologous SEAL1 TEP signature (Table 14) (right) upon transfecting TGFβ induced iMDFs with the mSeal1_G6 (mSeal1_6), mLrrc15_G3 (mLrrc15_3) or control Scrl ASOs. FIG. 20G shows a description of a pancreatic ductal adenocarcinoma (PDAC) mouse model involving the implantation of mouse PDAC tumor cells into DTR− or DTR+ genotype mice. DTR− genotype mice have a wild-type (wt) mSeal1/mLrrc15 genomic locus while DTR+ genotype mice have a heterogeneously modified (DTR-GFP cassette knock-in) mSeal1/mLrrc15 genomic locus, whereby both mSeal1 and mLrrc15 coding sequences are disrupted. In DTR+ genotype mice, diphtheria toxin (DT) is produced from the modified mSeal1/mLrrc15 genomic locus in cells where the mLrrc15 promoter is active, leading to these cells dying upon treating the DTR+ mice with DT. FIGS. 20H-20I shows scRNA-seq analysis of this mouse model. FIG. 20H shows fibroblast sub-clustering and the emergence of the different sub-clusters of fibroblasts with or without DTR at different timepoints (e.g., day 14, day 21). FIG. 20I shows expression of mSeal1 and mLrrc15 in the FBs of the dataset, with or without DTR at different timepoints (e.g., day 14, day 21).

FIG. 21A-21B show survival analysis of the TCGA-HNSC patient cohort (n=499). FIG. 21A shows a survival graph stratified based on myCAF fraction—High vs myCAF fraction—Low content. The myCAF fraction—High patient category had worse (p=0.0074) Overall Survival (OS) than the myCAF fraction—Low category, with a median survival of 2.5 years as compared to 4.5 years, respectively. FIG. 21B shows a survival graph stratified based on sub-clustering fibroblasts. The sub-cluster 1 myCAF fraction—High patient category had worse (p=0.02) Overall Survival (OS) than the sub-cluster 1 myCAF fraction—Low category.

FIGS. 22A-22B illustrate analysis of transcription factors (TFs) in CTRL vs TGF-β induced myCAFs, and the analysis of TF regulon expression in CTRL and TGF-β induced myCAFs with or without treatment with non-targeting (Scrl), SEAL1_G4 or LRRC15_G3 ASOs. FIG. 22A shows pseudotime plots of TEAD2, RUNX2, RUNX1, NFATC4 expression, activity, and positively associated TF regulon from HDFs to TGFβ induced CAFs. FIG. 22B shows Singscore analysis of the predicted target protein coding genes to the TFs TEAD2, NFATC4 and RUNX1 TFs in control HDFs vs TGF-β induced myCAFs treated with non-targeting (Scrl), SEAL1_G4 or LRRC15_G3 ASOs.

### DETAILED DESCRIPTION OF THE DISCLOSURE

Cancer-associated fibroblast (CAF), and particularly myofibroblast CAF (myCAF), plays a key role in tumor development. Compared to corresponding malignant cells within a certain tumor type, CAFs or myCAFs often display less interpatient heterogeneity across cancer types and patients, thus making them an ideal therapeutic target. However, the current CAF-directed therapeutics mainly target the protein-coding genes, and often suffer from a lack of target specificity. Therefore, there is an unmet need to develop more specific CAF-directed therapeutics.

Long non-coding RNAs (lncRNAs) are a special type of RNAs that are involved in all layers of transcriptional regulation covering a broad range of mechanisms. lncRNAs usually express in a cell type/cell state specific manner. However, the past research focused on lncRNAs expressed in malignant cells, rather than on lncRNAs expressed in CAF or myCAF. Therefore, it is an innovative therapeutic design to target lncRNAs expressed in CAF or myCAF.

With regards to novel lncRNA identification, previous studies were affected by several limitations. One of them was an absence of deep coverage RNA-seq data, which left a majority of lncRNAs to remain undetected as they may be expressed in low copy numbers. Another limitation was that the analysis of RNA-seq data lacking strand-specific read information hampers the detection of anti-sense lncRNAs that constitute a large portion of cancer-related lncRNAs. Therefore, the inventions disclosed herein present an unprecedented platform to identify novel lncRNAs that are specifically expressed in CAF or myCAF, and present a promising therapeutic target for cancer treatments.

In some aspects, provided herein are long noncoding transcripts that are associated with the development and/or activation of CAF, which can affect the state of the tumor microenvironment and development and prognosis of tumors (e.g., solid tumors). Such long noncoding transcripts could be a druggable target to prevent, alleviate, or treat development or prognosis of cancer, or symptoms of cancer. As such, in one aspect, provided herein, are modulators of a long noncoding transcript, pharmaceutical compositions comprising the modulator, and kits comprising the modulator. Also provided herein are methods of modulating expression or activity of a long noncoding transcript in a subject in need thereof using modulators of the long noncoding transcript. Further provided herein are methods of inhibiting a growth of a solid tumor or facilitating access to a solid tumor in a subject in need thereof using the modulators of a long noncoding transcript.

### Cancer-Associated Fibroblast (CAF)

Chronic fibrosis is one of the risk factors for cancer development. Fibrosis can lead to stiffened stroma or extracellular matrix, which enhances tumor cell growth, survival and migration. Development of tumor can further contribute to fibrosis and promote malignancy.

In some instances, cancer-associated fibroblast (CAF) plays pro-tumorigenic functions that contribute to the development of common and aggressive cancers as a mediator of tumor fibrosis (e.g., desmoplasia) in many cancers. Non-limiting examples of cancers affected by CAFs are breast cancer, colorectum cancer, lung cancer, prostate cancer, bladder cancer, head and neck cancer, skin cancer (e.g., skin melanoma), pancreatic cancer, liver cancer, esophageal cancer, brain cancer, stomach cancer, gallbladder cancer, or ovarian cancer.

CAFs can be derived from various cell types. In some instances, CAFs encompass a cell of non-tumor cell origin, wherein CAFs do not harbor tumor cell-characteristic genetic mutations or aberrations. In some instances, CAFs can be converted from fibroblasts, epithelial cells, endothelial cells, adipocytes, pericytes, stellate cells, bone marrow derived mesenchymal stem cells (MSCs), hematopoietic stem cells (HSCs), cancer stem cells (CSCs), CSC-like cells, or a combination thereof. In some instances, fibroblasts are converted to CAFs. In some instances, fibroblasts are converted to CAFs when fibroblasts are activated by growth factors (e.g., TGFβ, hepatocyte growth factor (HGF), platelet-derived growth factor (PDGF), or fibroblast growth factor (FGF). In some instances, fibroblasts are converted to CAFs when fibroblasts are activated by signaling proteins (e.g., Wnt-3A), transcription factors (e.g., NF-kB, HSF-1), cytokines (e.g., IL-2, IL-6), or combinations thereof. In some instances, activation of TGFβ/SMAD pathway, CXCL12/TGFβ1 pathway, PI3K/AKT pathway, MEK/ERK pathway, WNT/β-catenin pathway, GPR30/ERα pathway, TGFβ1/JAK/STAT3 pathway or combination thereof in fibroblasts contributes to convert fibroblasts to CAFs.

In some instances, CAFs are located in blood circulation. In some instances, CAFs are located in metastatic sites. In some instances, CAFs are located proximal to tumor cells. In some instances, CAFs are located distal to tumor cells.

In some aspects, CAFs have tumor-promoting functions. Non-limiting examples of tumor promoting functions include, tumor initiation, tumor proliferation, tumor invasion, tumor metastasis, apoptosis resistance, immunosuppression, metabolic reprogramming, therapeutic resistance, induce stromal stiffness, or angiogenesis. In some instances, CAFs modulates angiogenesis, wherein the CAFs enhances vessel density. In some instances, CAFs regulate expression and/or function of genes involved in angiogenesis (e.g., VEGF, MMPs, FGF). In some instances, CAFs play a role in development of desmoplasia that result in deposition of ECM. In some instances, CAFs regulate expression and/or function of genes involved in desmoplasia (e.g., hyaluronic acid, collagens, fibronectin). In some instances, CAFs modulates immune responses. In some instances, CAFs modulates immune responses by recruiting mast cells, polarizing T-helper cells, diminishing activity of natural killer cells, educating macrophages to act as tumor promoter, or combination thereof. In some instances, CAFs modulates contractile, ECM remodeling, ECM production or combinations thereof. In some instances, CAFs are involved in cancer metastasis formation or drug resistance. In some instances, CAFs are involved in cancer metastasis formation via Wnt2 and/or TGFβ. In some instances, CAFs are involved in drug resistance by regulating function of IL6, exosomes, SDF-1, Chi3L1, or combination thereof. In some instances, CAFs are involved in drug resistance by modulating epithelial-mesenchymal transition. In some instances, CAFs are associated with collagen-fibril organization, extracellular matrix (ECM) organization, tissue development in tumor microenvironment or combinations thereof. In some instances, CAFs modulates tumor-suppression.

CAFs are heterogenous with various sub-populations. Sub-populations of CAFs include, but not limited to, inflammatory CAFs, myofibroblast CAFs, CD146+ CAFs, CD146− CAFs, cancer-promoting CAFs, cancer-suppressing CAFs, vascular CAFs, matrix CAFs, cycling CAFs, or developmental CAFs. In some instances, CAFs comprise of inflammatory CAFs (iCAF). In some instances, iCAFs are converted from fibroblasts upon JAK/STAT/NFkB pathway activation. In some instances, iCAFs are located distal to tumor cells. In some instances, iCAFs modulates immune-modulatory. In some instances, iCAFs modulates developmental process, cellular process to growth factors, vasculature development, or combination thereof.

In some instances, CAFs comprise of myofibroblast CAFs (myCAFs). In some instances, myCAFs are converted from fibroblasts upon TGFβ pathway activation. In some instances, myCAFs are located proximal to tumor cells. In some instances, myCAFs modulates contractile, ECM remodeling, ECM production or combinations thereof. In some instances, myCAF differentially express a certain subset of genes that are not differentially expressed in fibroblast that is not associated with cancer. In some instances, myCAF can exhibit high expression of genes that are lowly expressed in fibroblasts that is not associated with cancer. In some instances, myCAF can exhibit low expression of a certain subset of genes that are highly expressed in fibroblasts that is not associated with cancer. In some instances, myCAF can exhibit high expression of a certain subset of genes that are lowly expressed in other sub-population of CAFs. In some instances, myCAF can exhibit low expression of a certain subset of genes that are highly expressed in other subpopulation of CAFs. In some instances, myCAF can exhibit a higher expression of one or more genes listed in Table 1 compared to a fibroblast that is not associated with or affected by a tumor cell, a tumor microenvironment, or development of cancer. In some instances, myCAF markers comprise ECM-modulatory gene. In some instances, myCAF can exhibit a higher expression of one or more ECM-modulatory gene when compared to a fibroblast that is not associated with cancer. Non-limiting examples of ECM-modulatory genes include LRRC15 (NM_001135057), MMP11 (NM 005940), COL11A1 (NM 080629), C1QTNF3 (NM_030945), CTHRC1 (NM_138455), COL12A1 (NM_004370), COL10A1 (NM 00493), COL5A2 (NM_000393), THBS2 (NM 003247), AEBP1 (NM 001129), ITGA11 (NM_001004439), PDPN (NM 006474), FAP (NM 004460), COL8A1 (NM_001850), COL1A1 (NM_000088), COL1A2 (NM 000089), FN1 (NM 212482), or POSTN (NM_006475). In some instances, higher expression of ECM-modulatory gene expression is associated with poor response to checkpoint blockade treatment, standard-of-care chemotherapy treatments of cancers, or a combination thereof. In some instances, higher expression of ECM-modulatory gene expression is associated with poor response to checkpoint blockade treatment of cancers.

TABLE 1
Markers of myCAFs
Gene Name

MMP11, COL11A1, C1QTNF3, CTHRC1, COL1A1, COL12A1, COL10A1, FN1, COL1A2,
SDC1, COL5A2, GJB2, LGALS1, TPM1, COL3A1, THBS2, HTRA1, SPARC, CD99,
AEBP1, NBL1, ACTB, MFAP2, TMSB10, LRRC15, MMP14, PLAU, INHBA, POSTN, ITGA11,
ANTXR1, PLPP4, MYL9, COL5A1, MFAP5, ATP5E, IGFL2, F13A1, CD55, GREM1,
COL8A1, MYL6, ARL4C, MXRA5, CALM2, PRSS23, MARCKS, SERPINH1, SUGCT,
SULF1, FNDC1, CALU, CAPZB, ADAM12, GJA1, IGFBP3, SPATS2L, ANXA2, CMTM3,
HCFC1R1, PTK7, ITGB5, CFL1, RUNX2, LOXL2, ID3, LOXL1, CTSK, RGS16, COL8A2,
CDH11, C5orf46, VCAN, FZD1, TPM4, NTM, TAGLN, RAB31, TPM2, NUAK1, KIAA1217,
CAVIN3, RIN2, SOX4, PALLD, PKM, CALD1, ACTA2, EPYC, RARRES2, MATN3,
MYL12A, MMP7, NREP, EDIL3, TMED9, GAS1, PXDN, DERL3, ACTG2, OST4,
S100A16, CNN2, VGLL4, S100A11, P4HB, COL6A3, IL32, MYH9, CARHSP1, SFRP2,
GALNT1, HSPA1A, ENAH, SLC44A1, PDLIM5, SERPINA1, C4orf48, LPP, OSTC, DIO2,
FLNA, RCN3, PDLIM2, PDZK1IP1, KCNQ1OT, KRT7, FBXO32, CTNNB1,
CXCL14, TUBA1A, HSPA1B, MDK, MYLK, ISG15, KRT19, IGLC2, SLC6A6, IFI27,
LUM, MT-RNR1, SPINK1, IGKC, IER2

CAF lncRNAs

Long non-coding transcripts (or long non-coding RNAs (lncRNAs)) are RNA segments that lack protein-coding capacity, yet, in some instances, can mediate various regulatory mechanisms in cell cycle or cell metabolism by regulating transcription and/or post-transcriptional modification of various genes. As such dysregulation of certain long non-coding transcripts can be associated with an onset, development, or prognosis of a disease or a symptom of a disease. Alternatively and/or additionally, dysregulation of certain long non-coding transcripts can be a signature or indication of an onset, development, or prognosis of a disease or a symptom of a disease.

In some instances, the expression or activity of lncRNA disclosed herein is upregulated in tumor tissues compared to normal tissues. As used herein, normal tissue includes (e.g., non-cancerous tissue, non-precancerous tissue, a tissue that is not within the cancer microenvironment, etc.) of the same individual, or a tissue from a healthy individual. In some instances, the lncRNA disclosed herein have substantially no or low expression across normal tissues (e.g., in vitro, ex vivo, or in vivo). In some instances, the lncRNA disclosed herein have substantially no or low expression across normal human tissues (e.g., in vitro, ex vivo, or in vivo tissues). In some instances, the lncRNA disclosed herein have substantially no or low expression across normal cells (e.g., in vitro, ex vivo, or in vivo). In some instances, the lncRNA disclosed herein have substantially no or low expression across normal human cells (e.g., in vitro, ex vivo, or in vivo cells).

In some aspects, the lncRNA disclosed herein is associated with an activation of CAF.

The term “activation” refers to a situation where fibroblasts are differentiated, converted, or transitioned to CAFs. In some cases, “activation” refers to the change in morphology, gene expression pattern, and/or an element of a signaling pathway of fibroblasts, wherein the change reflects morphology, gene expression pattern, and/or signaling pathways of CAFs. In some instances, morphological changes associated with myCAF activation may involve, but are not limited to, a flattened cell shape and a connective, swirling growth pattern. In some instances, the lncRNA disclosed herein regulates the expression and/or function of growth factors (e.g., TGFβ, hepatocyte growth factor (HGF), platelet-derived growth factor (PDGF), or fibroblast growth factor (FGF) to activate fibroblasts to CAFs. In some instances, the lncRNA disclosed herein regulates the expression and/or function of signaling proteins (e.g., Wnt-3A), transcription factors (e.g., NF-kB, HSF-1), cytokines (e.g., IL-2, IL-6), or combinations thereof to activate fibroblasts to CAFs. In some instances, activation of TGFβ/SMAD pathway, CXCL12/TGFβ1 pathway, PI3K/AKT pathway, MEK/ERK pathway, WNT/β-catenin pathway, GPR30/ERu pathway, TGFβ1/JAK/STAT3 pathway or combination thereof in fibroblasts converts fibroblasts to CAFs.

In some instances, the expression or activity of lncRNA disclosed herein is upregulated in CAF. In some instances, the expression or activity of the lncRNA disclosed herein is increased in CAF compared to fibroblast that is not associated with cancer (e.g., non-cancerous or non-precancerous fibroblast, fibroblast that is not within or nearby the tumor microenvironment, etc.).

In some instances, the transcription of the lncRNA disclosed herein is enhanced in CAF compared to fibroblast that is not associated with cancer. In some instances, the expression or activity of the lncRNA disclosed herein is decreased in CAF compared to fibroblast that is not associated with cancer. In some instances, the transcription of the lncRNA disclosed herein is decreased in CAF compared to fibroblast that is not associated with cancer. In some instances, the expression or activity of lncRNA disclosed herein is upregulated in human dermal fibroblast (HDF) treated with TGFβ with or without starvation. In some instances, the expression or activity of lncRNA disclosed herein is upregulated in myCAFs. In some instances, the expression or activity of the lncRNA disclosed herein is increased in myCAF compared to fibroblast that is not associated with cancer. In some instances, the transcription of the lncRNA disclosed herein is increased in myCAF compared to fibroblast that is not associated with cancer. In some instances, the expression or activity of the lncRNA disclosed herein is decreased in myCAF compared to fibroblast that is not associated with cancer. In some instances, the transcription of the lncRNA disclosed herein is decreased in myCAF compared to fibroblast that is not associated with cancer. In some instances, the expression or activity of lncRNA disclosed herein is specific to myCAFs. In some instances, the expression or activity of lncRNA disclosed herein is upregulated in myCAFs from head and neck squamous cell carcinomas. In some instances, the RNA expression level of the lncRNA disclosed herein is at least about 10%, at least about 20%, at least about 30%, at least about 40%, at least about 50%, at least about 60%, at least about 70%, at least about 80%, or at least about 90% more than in a myCAF compared to fibroblast that is not associated with cancer. In some instances, the RNA expression level of the lncRNA disclosed herein is at least about 1.5 folds, at least about 2 folds, at least about 3 folds, at least about 4 folds, at least about 5 folds, at least about 6 folds, at least about 7 folds, at least about 8 folds, at least about 9 folds, or at least about 10 folds higher in a myCAF compared to fibroblast that is not associated with cancer.

In some instances, the lncRNA disclosed herein is identified by comparing sequencing data of myCAF to sequencing data of fibroblast that is not associated with cancer, wherein the data is produced via Nanopore direct RNA sequencing. In some instances, the lncRNA disclosed herein is identified by comparing sequencing data of myCAF to sequencing data of fibroblast that is not associated with cancer, wherein the data produced via total RNA-Seq. In some instances, the DNA variants of the lncRNA described herein are identified by comparing sequencing data of myCAF to sequencing data of fibroblast that is not associated with cancer, wherein the data is produced via polyA-selected RNA-seq. In some instances, the lncRNA disclosed herein is identified by comparing data from myCAF to data from fibroblast that is not associated with cancer, wherein the data is produced via GWAS and common variant calling. In some instances, the lncRNA disclosed herein is identified by comparing data from myCAF to data from fibroblast that is not associated with cancer, wherein the data is produced via CUT and RUN probing, for example, histone modifications or transcription factors. In some instances, lncRNA disclosed herein is identified by comparing data from myCAF to data from fibroblast that is not associated with cancer, wherein the data is produced via ChIP-Seq probing, for example, histone modifications or transcription factors. In some instances, the lncRNA disclosed herein is identified by comparing data from myCAF to data from fibroblast that is not associated with cancer, wherein the data is produced via single-cell RNA seq (scRNA-seq). In some instances, the lncRNA disclosed herein is identified by comparing data from myCAF to data from fibroblast that is not associated with cancer, wherein the data is produced via bulk ATAC-seq.

In some aspects, the genomic region that is transcribed to one or more lncRNAs disclosed herein is located within chr3:194355288 to chr3:194370349 (+). In some instances, the genomic region that is transcribed to the lncRNA disclosed herein is located within chr3:194355288 to chr3:194358966 (+). In some instances, the genomic region that is transcribed to the lncRNA disclosed herein is located within chr3:194368496 to chr3:194370349 (+). In some aspects, the genomic region that is transcribed to the lncRNA disclosed herein is located within chr6:74958809 to chr6:75026433 (−). In some aspects, the genomic region that is transcribed to the lncRNA disclosed herein is located within chr1:66390975 to chr1:66516344 (−). In some aspects, the genomic region that is transcribed to the lncRNA disclosed herein is located within chrl2:67394371 to chrl2:67455635 (+). In some aspects, the genomic region that is transcribed to the lncRNA disclosed herein is located within chrl2:67394371 to chrl2:67590771. In some instances, the lncRNA ENSG00000203585 is transcribed from a genomic region located within chrl2: 67394371-67590771 (+). In some instances, the lncRNA SHARED_00113753 is transcribed from a genomic region located within chrl2: 67394371-67455635 (+). In some aspects, the genomic region that is transcribed to the lncRNA disclosed herein is located within chr9:87219871 to chr9:87277312 (−). In some aspects, the genomic region that is transcribed to the lncRNA disclosed herein is located within chr2:215718043 to chr2:215720944 (+). In some aspects, the genomic region that is transcribed to the lncRNA disclosed herein is located within chr1:230710698 to chr1:230795492 (−). In some instances, the genomic region that is transcribed to the lncRNA disclosed herein is located on the sense strand. In some instances, the genomic region that is transcribed to the lncRNA disclosed herein is located on the anti-sense strand. In some instances, the lncRNA disclosed herein comprises XLOC_055514, XLOC_055515, XLOC_069921, XLOC_005184, ENSG00000203585, SHARED_00113753, or a fragment thereof. In some instances, the lncRNA comprises XLOC_055514, XLOC_055515, XLOC_069921, XLOC_005184, ENSG00000203585, ENSG00000288903, ENSG00000230838 (LINC01614), ENSG00000244137, or a fragment thereof. In some aspects, the lncRNA disclosed herein comprises a sequence set forth in Table 11. In some instances, the sequence of the lncRNA disclosed herein can be found in a public database (e.g., Ensembl, ensemble.org/Homosapiens/). In some aspects, the lncRNA described herein is transcribed from a genomic region listed in Table 10 or Table 12. In some aspect, the lncRNA described herein is transcribed from a subset of a genomic region listed in Table 10 or Table 12. In some aspects, the lncRNA described herein comprises a sequence listed in Table 11. In some aspects, the lncRNA described herein comprises a fragment of a sequence listed in Table 11. In some aspects, the lncRNA described herein shares at least 50%, at least 60%, at least 70%, at least 80%, 85%, 90% or 95% sequence homology with a full-length sequence listed in Table 11. In some aspects, the lncRNA described herein shares at least 50%, at least 60%, at least 70%, at least 80%, 85%, 90% or 95% sequence homology with a fragment of a sequence listed in Table 11.

TABLE 10
Genomic regions (coordinates with strand information)
which encode exemplary lncRNAs associated with myCAF

| lncRNA name | SEAL# | Genomic coordinates (strand) |
|---|---|---|
| XLOC_055514 | SEAL1 | chr3: 194355288-194358966 (+) |
| XLOC_055515 | SEAL9 | chr3: 194368496-194370349 (+) |
| XLOC_069921 | SEAL2 | chr6: 74958809-75026433 (−) |
| XLOC_005184 | SEAL3 | chr1: 66390975-66516344 (−) |
| ENSG00000203585/ | SEAL4 | chr12: 67394371-67590771 (+) |
| SHARED_00113753 | | |
| ENSG00000288903 | SEAL5 | chr9: 87219871-87277312 (−) |
| ENSG00000230838 | SEAL6 | chr2: 215718043-215720944 (+) |
| (LINC01614) | | |
| ENSG00000244137 | SEAL7 | chr1: 230710698-230795492 (−) |

TABLE 11
Sequence of XLOC_055514, XLOC_055515, XLOC_069921, XLOC_005184, and SHARED_00113753

| SEQ | | |
|---|---|---|
| ID | | |
| NO. | Sequence | Description |
| 2060 | CCCCUCCGGGCCUCUGUAGACUCAGUUAGUCCACAGCCUGCUCACUUCGU | RNA |
| | GGGAAUAGUUCUCCGCUGAGAUAGCCCCUCUCGCCUAAGUAUUAUGUAAG | sequence |
| | UUGAUUUCCCUUCUUUUGUUUCUCUUGUUUGUGCUACGGCUUGACCCAGC | of |
| | AUGUCCCCUCAAAUGAAAGUUCUCCCCUUGAUUUUCUGCUCCUGAAGGCA | XLOC_ |
| | GGGUGAGUUCUCUCCUCAAAGAAGACUUCAAACCAUUUAACUGGUUUCUU | 055514 |
| | AAGAGCCGUCAAUCAGCCUGGUUUUGGGGAUGCUAUGAAAGAGAGAAGGA | |
| | AAAUCAUGCCGCUCAGUUCCUGGAGACAGAAGAGCCGUCAUCAGUGUCUC | |
| | ACUUGUGAUUUUUAUCUGGAAAAGGAAGAAACACCCCAGCACAGCAAGCU | |
| | CAGCCUUUUAGAGAAGGAUAUUUCCAAACUGCAAACUUUGCUUUGAAAAG | |
| | UUUAGCCCUUUAAGGAAUGAAAUCAUGUAGAAUUUUGGACUUCUAAAAAC | |
| | AUUAAAAUCAGCUUAUUAAUACGGGAUAGAGAAAGAAAUCUGGUGCCUGG | |
| | GGGUCCCUGUGUUCACCCCUAGAGUUUGUUUUAAAAUUUUUAAUUGAAGC | |
| | AUGUGAAGUGUACGUGCAGAAAAGUGGGAACAUGAUAGUGUAUGGCUUGG | |
| | UGGAUUUUCACAAACUGAACAUACCUGUGUAAUCAGCAUCUAGACCCAGA | |
| | CCCAGAGCAUCACAAAUAUCCCCCAUCCUGGGCUUUUCCCAGAGGAGAUG | |
| | GGGGCUUCUGAAGAUGGACUUACCUGGGACCUGCCCCCCAUGAGCCAGGA | |
| | CGGUCCCCCCACAGUCAGCCUGUGCAAAGGCCCCGUGGCCAGGGGUGGAG | |
| | GAGAAUAUGUGGGUGUGGACAGGAUGGGAGACUGUGGCCUGAACAGGAGA | |
| | UUUUAUUAUAUCUGGAGACCCUGAGAGACCCUGAGACCUGGGGCACCAUG | |
| | GCUGGCCAGGUCAGAAGCAUCCUGACUGCAGAGGUCCGUGCAGCCACACC | |
| | CUCUUCCCUGCCAGCAAGCUGUCUGCGGCUCAUCGGAGGCCCCUCCGCCU | |
| | GGAGCCUUCUAUGGACGUGAUAUGCCUGUAUCUGUUUUUAAUUUUCAUUC | |
| | UUCACUUAGGGGAAGUGAAAUCGCUCAGAGAUGAGAUCCUUUAAUUGAAA | |
| | ACGAAGUGUAACGGAAUCUAGUGUCUUUCUAAUGUGGUAAAAUUCUCCAU | |
| | CAACAUCACAGUCAGCUGGCAGCUGAACUUCAGAAUCUCACUUACAGCAG | |
| | GCGACACGGGGGUACACCGAUGGGUCACACUGGGUCUGGGGGCUCCCUGG | |
| | AGCUCCUCCUGCGUGUGGUCUGGUUAGGAGUUGAGUUGUUUGCUCCAGGG | |
| | UUAUUCUCCUCCUCGAGUCACAGUCACACGAAUACCUGCCUUCUCUGGCU | |
| | UUCCUGCUAUACACAUAUUCACAUGGCGCUCAAGAAGUUAGGCUCAUGGC | |
| | AACGUGUGUCUUUCUCUGGACAACUGGCCCAGUUUACAGUGAAAUGGAGA | |
| | AUUUCAGGUCUCCACGUCUGCCCAGGAAAGAACUUCAGCUGACUCCACGG | |
| | GGAUCUGGAAAUCCACGACCAAUCCCGAUCGGCUCUUAUUAGCUCCCCGC | |
| | UCCACAAGACACCUGUGCUUUGGAAAUCCACCACCAAUCCCGAUCGGCUC | |
| | UUAUUAGCUCCCCGCUCCACAAGACACCUGUGAUCUGGAAAUCUACCACC | |
| | AAUCCCGAUCGGCUCUUAUUAGCUCCCCGCUCCACAAGACACCUGUGACA | |
| | UCCUCCAGGGCCACAGGAGCACGUGCUGACCAGUUUUCCCUUCCAGUUCC | |
| | UGCACAAAAAGUGUCCAGAGGGCUGUUUGCAAACACUAGUGCACUUUGUA | |
| | GCUUUUCACCCUCUGUCCCAGGGAAUCUAGGAGAGAUGAGGCCCGUCAGA | |
| | GUCAAGAGAUGUCAUCCCCCCAGGGUCUCCAAGGCAUUUCCACACUAUUG | |
| | GUGGCACCUGGAGGACAUGCACCAAGGCUUGCCAGAGCCAACAGGAAGUG | |
| | AGCCCAGAGCAUGGCACAUGAGCAUCACCCGCUGAUGGUGGCCUGCUGUG | |
| | CCUGGUGCCAACAGGGGCAUCCCGGCCCGUACCCCUCCAGACAGGAAGCA | |
| | UGGGUUUGCCCACAGACCUGUCGGGUGCUCCUGUGAGUGGCCUCCAGAUG | |
| | UCUUUGUGCAUAGGCACAAGUGGGCCAGGGCUGGAGGGAGGUGGGAAACC | |
| | UCAUCAUCCGGUGGGCCCUGCCAAUCUUAACCCAGAACCCUUAGGUAUUC | |
| | CUGGCAGUAGCCAUGACAUUGGAGCACCUUCCUCUCCAGCCAGAGGCUGA | |
| | CCUGAGGGCCACUGUCCUCAGAUGACACCACCCAGGAGCACCCUAGGUGA | |
| | GGGGUGAGGGCCCCCUUAUGUGAACCUCUUGCCUCUUCCUUUCUCCCAUC | |
| | AGAGUGGUUGGAUGGAGCCAUUGGCCUCCUUUUCUUCAGCGGGCCCUUCA | |
| | ACCUCUCUGCACCAUGUUGUCUGGCUGAGGAGCUACUAGAAAAGCUGAGU | |
| | GGAGUCUCCUUUCCAACAGGAUGAUGCAUUUGCUCAAUUCUCAGGGCUGG | |
| | AAUGAGCCGGCUGGUCCCCCAGAAAGCUGGAGUGGGGUACAGAGUUCAGU | |
| | UUUCCUCUCUGUUUACAGCUCCUUGACAGUCCCACGCCCAUCUGGAGUGG | |
| | GAGCUGGGAGUCAGUGUUGGAGAAGAAACAACAAAAGCCAAUUAGAACCA | |
| | CUAUUUUUAAAAAGUGCUUACUGUGCACAGAUACUCUUCAAGCACUGGAC | |
| | GUGGAUUCUCUCUCUAGCCCUCAGCACCCCUGCGGUAGGAGUGCCGCCUC | |
| | UACCCACUUGUGAUGGGGUACAGAGGCACUUGCUCUUCUGCAUGGUGUUC | |
| | AAUAGGCUGGGAGUUUUAUUUAUCUCUUCAAACUUUGUACAAGAGCUCAU | |
| | GGCUUGUCUUGGGCUUUCGUCAUUAAACCAAAGGAAAUGGAAGCCAUUCC | |
| | CCUGUUGCUCUCCUUAGUCUUGGUCAUCAGAACCUCACUUGGUACCAUAU | |
| | AGAUCAAAAGCUUUGUAACCACAGGAAAAAAUAAACUCUUCCAUCCCUUA | |
| | AAGAAUAGAAUAGUUUGUCCCUCUCAUGGGAAUUGGGCUGUAUGUAUAUU | |
| | GUUCUUCCUCCUUAGAAUUUAGAGAUACAAGAGUUCUACUUAGAACUUUU | |
| | CAUGGACACAAUUUCCACAACCUUUCAGAUGCUGAUGUAGAGCUAUUGGG | |
| | AAAGAACUUCCAAACUCAGGAAGUUUGCAGAGAGCAGACAGCUAGAGAUA | |
| | ACUCGGGACCCAGAGUUGGUCGACAGAUGUUAGAUGUAUCCUAGCUUUUA | |
| | GCUAUAAACCACUCAAAGAUUCAGCCCCCAGAUCCCACAGUCAGAACUGA | |
| | AUCUGCGUUGUUGGGAAGCCAGCAGUGGCCUUGGGAAGGAAGCCAUGGCU | |
| | GUGGUUCAGAGAGGGUGGGCUGGCAAGCCACUUCCGGGGAAAACUCCUUC | |
| | CGCCCCAGGUUUCUUCUUCUCUUAAGGAGAGAUUGUUCUCACCAACCCGC | |
| | UGCCUUCAUGCUGCCUUCAAAGCUAGAUCAUGUUUGCCUUGCUUAGAGAA | |
| | UUACUGCAAAUCAGCCCCAGUGCUUGGCGAUGCAUUUACAGAUUUCUAGG | |
| | CCCUCAGGGUUUUGUAGAGUGUGAGCCCUGGUGGGCAGGGUUGGGGGGUC | |
| | UGUCUUCUGCUGGAUGCUGCUUGUAAUCC | |
| 2061 | UUUUUAGAGGGGACACAGUGGUCGUAAUUAUAUUUUUCCUUGGAAUCUAA | RNA |
| | AGUAAAUGCUUCUUCCAAAAUGUUAACAACAGAUGAAACACAUUAAUUGU | sequence |
| | CUGUAAAUUUAAAGCCAAAUAAAAAGUUUAAAAUAAGAAAGAAAGAAAGA | of |
| | AAGAAAGAGAAGAAUGAAUAAGUUUUCUAGAACCACAGGGAGAAGCACUU | XLOC_ |
| | GCCAAUACUUCUAGGAGCAAAGAAACGCCCCUGAAUCCUGCUCCAUCCCU | 055515 |
| | UUCUCGGGGUCCUUGUCUCCUUGGCCAGGGCAGGGCAGCAGGGGGUCCAC | |
| | AGUCACUUAGGGAGGGGUUUACCGGGUCGUUGUAAACUCCCCUCUCACGG | |
| | UGGCCAAUGCAGUGAAGGAAUCCAAAAGAUACACUGGGGGAAAGAAGGAA | |
| | AGAGGAAAAUGUGUCCUGAGUUUUUAAAUACGGUUGAAGCAAACCCCUUA | |
| | CAUUUAGGGAUGCCAGCUAUGUGUACCCUGUAGUGUCAGCCCGGGACAUG | |
| | CAGGCACUGCACUGUGGCCCACAGUCCCCCCUCCCAGGGCCAACACUGCC | |
| | CUCUGCAGUGACUCACGCCCGGGAUAGUUUAAAUACCGGGACAGCUUAGG | |
| | CGAGCCACACUCAUGCUGCAGCCUUGAGCCGUCCCUCGUCCUCCUCUCAG | |
| | GCUCCCUCUUGUCCACGGCGGGCGGGCGCCGAGCUGCUGGUAAGUAGGCA | |
| | AGCCACCCCCAUCCCUCCUCUCUGCUUAGAGGGCCCGGCCAGCCCCUUCC | |
| | CAUUUCUGCUGGACCUGCUCCCCUCGGAGACCCCAGAAGGGGCAGAGCUG | |
| | ACAUGUGCCUGGGGAUGCUCUCGGGACAGGGACAGAAGCAGCCUGUGCUG | |
| | GAGGGGUGCAAGCCUGUCUCCCUCACUGCCUGGACACCAGGACCACCAGC | |
| | CCCUCUGCUGAUGCCUCAACAGCCUCUUAUGCCCCUUGCCCUGAGCGGCU | |
| | CAUGAUGUCAACCUGGGGUGCUGAGAUGUCUGAGACCAGCAAGUUGGGCU | |
| | GAAAGUGGACUAAAUUAUCACCCCCCUUGUCACUGGAUUGACCGUUCUGG | |
| | AAUCUAGAUCCACUGGGGGAAGAACUUCUCUUUUUAAGGUUCAGAUUAUU | |
| | UGCGCCUGUGCCAUCUUUCCUGGCACUUGCCCUGGUGGCUCCCAGUGGUC | |
| | UGCCCCGCAUUUGGUUCUGCCCAAGACCUUCCUUCAUGGGGGUUUCCCAG | |
| | GCAAGAGAGCUGUCUGCUUUGCAUAGAUGGAUAACUCCUGCAAGUGUAAC | |
| | UUCAGCAGAUGUGCUGGUCCCUGGUUUGCAGUUCCAAUUUGGAAUUCGGA | |
| | CAUGGGCUCAGCAUUGUUCAGGCUCUAACAGUGUGGCUUAGGGGACACUA | |
| | UGCUGGCAGUUUCCCCUCUCUCCUGCUGGAAGCGUCGGCCUCUGUGGAGC | |
| | CUGGAGGCUUUGAGCAGGGAGUCAGGGCAGGAGAUGGAUUUGUGAUGAGG | |
| | UGGUAUGUGGAUUUGAAAUUGUGACGGCUGCACCUUGACCCGUGUCUCUG | |
| | CUCAAACGUAGAGGCAGGACCUGGGGCCAAGAAACCAACUGUGGAAAACC | |
| | AGAGCUUUCAGGACUCUCCCACCCCUGCCAAAGUGCAAGAGCCAUUCCAC | |
| | CUCCCGGGCCCAGGGCAUCACCUCGGUGCAGCUGCACUCUCUCUGAGAUG | |
| | ACACGUGUGCUCCAGUCGCAGGUGCCAGGUGCCUGUCUGCCACGUGCCAG | |
| | CUUCUCCUUUGGGGUUAUCUGGGAUGCGUCACUUUACCUUGGGCAGCUCU | |
| | CAGAUGUGUUGCUGCCACUGGCUCCUUAAUUUAGAAGUGCUCUAACGGCU | |
| | GGCACUUCCUUCAUUCUGCAGAGACUGAACAUCUGGAGUAAAUUCAGGGC | |
| | CAGA | |
| 2062 | UAAAGCCAGAAUUCUCCUGCCAGGAUUCCAUCAGGAGUUACCUGCUGGCU | RNA |
| | CGAUGUCCCAGUCUGUGCUUAAAGCAGGAUGAGAAAGCUUUGCCACAGGA | sequence |
| | AAUGAGAUAAUGCAUGUACAAACUCAUUUAUCUUCUCAGGAAAAUACAUU | of |
| | ACUUUAAGUUGUAUCUUUAUGCUCUACACAUUUUAACAGACACACAGCAG | XLOC_ |
| | UUACCCUUGGUUUUUCUUCUUUACUGUCACUGAUUCACAAUCAUAAUUAA | 069921 |
| | AAAUGCCAAACGCAUCUUUAUGUGAAACAAAAUAAUUUUUUUCUUAUUUU | |
| | UAUAGACAAAACCGUCCAAAAUGACAUAUUUUUCUCCAGGAAAGACAGCA | |
| | UGUUGGUGUAGGAAACAGAGUUAGUGUGCUAUGGAGUUAGAAGACCUGGA | |
| | UUUCACCAUUUAAAAGCCAUGUGAACUUGAGCCAGUCCCUGAACCUGUUU | |
| | AUUCAUCUAUUAAAUGAAAAAUAGGAAUCUCUCUAAUUCAUGAAAACUUU | |
| | GAGAAGAUUAAAUUAGACAAGAUGAAUGAAAAGAAUUCCAAAUAUACAAA | |
| | UCACAUGCAAACAAGUCUUGGCUUAUUAGUCUACUGGGAGCAGAUUACUA | |
| | CUUUGAAUCUUAAUGCCCUCUCUUUCAGGUUAAUAUGCAAAAAUACACAC | |
| | AAAACACUUAAAAUGUAAAAAGAGGUAGUGACAUCACUUGCUGACUUAAG | |
| | UCUGAACUCAAAUUGGUUAACCUGCCUAUAUAUGGAAAAAUUUCCAUUUA | |
| | AAUGAAAAUAUUUCACUAGCCCACAAACAGCCUUAGACGUGGUUUCCUGU | |
| | UUACCUUUAAAUGGAUGUUGUGGUUUCUUCUUAAUGAUUGGAGGAAGUUC | |
| | CAAAUUUUGGUGAAAUGAUCUCAUUUAAACAUAGAGCAGGAUAUGAGAAA | |
| | GAAUGAUUAACUUGUGGUUCCGAUUGAUGUUGUAUAUUUUACACACAGCU | |
| | GGAUAAGUCUAUUGUAAUAAUUAAAAAUUUAUUUCACUAAUUUUUUAUUU | |
| | UCAUGAAUAUUAAUUAUUAUGUCCUAAGCAUUGUUGUAGAGAUUAAUAAU | |
| | GACAAUAAUGAAGAAGAUAUUGUCCUUGCUUUCAAGGAGUUGUUAAUUUA | |
| | GUUUAGAAAAUAGGCAAGGAAAUCAGUGAUUGCAGUAUAAAGUGGUAACU | |
| | GUCUUUAGAAGUUGGUGUGAUCUGUUUGAUAUUAGAUGUUUCUUGGCUGC | |
| | UACUUCUGGAGUAAUUUUUAACGGUGCUAUGUAAGAAUUCCAUUGUUCAC | |
| | AUAAGUAUGCAAUGGAAGGUCUACUUUUCAGCUCAGAUUCAUUUAUUGUU | |
| | UCUCACCCUCUUUCCACAACCCAGAAGGUACUUAGUGAGUCUCCUUAAGU | |
| | GACAAAUAGUAUUUGACUCUUUCCAUUAGAAUUACUCAUGAUUUCUUUUC | |
| | CCUUGUCUAAUAUUCACAAGAUUUCUGCUAGAACAGAGAGAAUACAGUCC | |
| | UCAGAAAUACAAACACAAAUUAUUUAGUUUAGUUAAAUUAUAAGGCUGCA | |
| | GCAUUGCAUACACACAGGCAGUAUUUUGUUGAGUAAAAUGGCAGGAAGCC | |
| | AAAACUUUUAUAGAAACCCGCUGAUAAUUCAACACUUAAAAACUAUGAUU | |
| | UAAAAAUGUACAAGUGAAAGAUGAUUUCUUAGAAAUGCAGUAAUCUUUUA | |
| | GCAACCAUAUCAAUGCUGAUAGCAGGGGACAAGAGGGUCACAAAGAGAUU | |
| | CCAGAAAAGAAUAGGUUCUUUGUCUUGCUUACUCUGGGUUAGUUAUUACC | |
| | AGUUUUCUGGUAAGGAAGAAUUAGAGGUAAAUAUCAGAACUUACUAAACA | |
| | UACCAUAGGUCACUGUUUAGUGGCCACUAUUUUCACAGGGGUAUGUGAAA | |
| | AAUGUAACUCCUUCCCUUUUCUUGCAUAUCAAUUUGAGCUCACUUAGCCA | |
| | GCCCUCUUUCUGCUAGAUAGUAGGAAACUUUAUCUAGAUAAUUCCAGGUG | |
| | AGCCUAGACAUCAUCCAAUGUUUCACCAAGUGUUUUUUUUUAAUUCCCAU | |
| | UUCUCUGUCCUUUUCUUCAUGAAAUCAUCUAAGAUUGGCUAGGGGCGGUG | |
| | GCUCACGCCUGUAAUCCCAGCACUUUGGGAGGCCGAGGUGGGCGGAUCAC | |
| | GAGGUCAGGAGAUCAAGACGAUCCUGGCUAACACGGUGAAACCCUGUCUC | |
| | UACUAAAAAUACAAAAAAUUAGCUGGGCAUGGUGGCAGAUGCCUGUAGUC | |
| | CCAGCCACUCGGGAGUCUGAGGCAGGAGAAUGGCAUGAACCCGGGAGGUG | |
| | GAGCUUGCAAUGAGUGAAGAUUGGGCCACUGUACUCCAGUCUGGGCGACA | |
| | GAGCAAGACUCCAUCUCAAAAAAAAAAAAAAAAAAAAAAAAAAGAAAGCA | |
| | UCUAAGAUCUCCAGCUUUGGCAGAACUAUCAAUCCAUGUUGGCACCUGUC | |
| | CAGAUGCCUGUGUUUGCCUGUCUGAGCUCUGCUCCUGGUGACAUGGCCUC | |
| | UCCAGAUCUACAGAUUGUUUCUUAGCUAGUAUCAGGCUCCUUUGCUAUUU | |
| | UACUUGGCACUUUAAGAUGGGACUGUGGAAGAUUCUCUGGGCUACAGGCC | |
| | AGCUGGCCAGGCUCCCCUGCAGCCAUUCUGUCCCCACUUUACCAGGGAGG | |
| | UAGGAGAAACUUUGGCAAGGUUGCCACCCUUACAGUCCGGACAGGCAGAC | |
| | UGUACACUUCUUCUACUCUGGGACUCCUCUAAAUCUAUCUUUCAGUAGAG | |
| | AUCUGCCUGGGGCACCUAAAAGGUGCCCUCCACCUUCUAGGACCUAAGUG | |
| | UUUUUAGGUAGUUUUGGGGUAGAAGGUGGUGAUAAAAAAUUCCCAUGUUA | |
| | UCACAUUUUCCUUCAAAUAUUACUAUGAUACAGCUAGACUGUGCUUUCAA | |
| | AAUGAAAAUCUGCGAAUUUUAUGUCUCGUUUGUCCCCUGAUUCUGCACAC | |
| | CUUGGGGUUAAUGGAAGACUUAUAUUUAGUUCUGUCAGGUAGUAUGAAAA | |
| | AACAUGGUACUGUGAAGAGAAGCAGCUUUUCUUUACAACGUGCAUGGUGA | |
| | UUCUUGAGAAUUCUGCUGUUGUCUUCAUAAAUUUCUGGGGUUAGUAAUGC | |
| | CUCCAGGUGUCUUUGCAGGUAAAUAUGUCUACUAGGUUUCCUUUCAGAUG | |
| | CAGCUUUAAUGCUUAGGGCUGGUAAAGCUGGUAAGAACUAUUCUUGGAGA | |
| | AUUCCAUCUAACAGUUUAUCUGUGUCUAGAGUAGUAACAGUUGCUUAUGU | |
| | GAAGAGAACAAAGAAUUACAGAAGAGGAAUUAUGUUAACAUUUUACUUCA | |
| | GAGACAGGUUUAAUUUAUUUUACUACAUGAGCCCCUUUAGAUACGGAGCU | |
| | GAUAGUAAUUUCUUGCUGAAAGAACUAAAACGACCACUUCAUUGGAAACU | |
| | CAUUAAGGAAAAUGCCUGUGUAUUAUAACAUCACCUUAGUCCUUACUUGU | |
| | AGAAGAGCACAUAAUAGUCCCUUAAAUUUUAUUGAAUGAGUAGAAUUUCA | |
| | GGCUACAAUGUACCAACUUGCCCUAUAAAUUUUGUCAGGUAAUGUUGGUA | |
| | UAUUUUUCAAACACAUUUCUUAGGACUAUCAAAAAAAAGCAGAGACAGAA | |
| | AUUAAAGUGGUAAAGGGAAAUUUUAUUCAGGACUAUUGCAAUAGGGGAAG | |
| | AGAGACCCCCAUAUAGAACUGGGCUUCAUUCUAAAUACAUCAGGGAUAAG | |
| | UAGGGAUUUAUAGCCAAAGAGCAGGGAUCAGAAAAUUUUUAUGUAUUUAU | |
| | UUAUUUAUUUAUUUAUUAUUAUUAUACUUUAAGUUUUAGGGUACAUGUGC | |
| | ACAAUGUGCAGGUUAGUUACAUAUGUAUACAAGUGCCAUGCUGGUGCGCU | |
| | GCACCCACUAACUCAUCAUCUAGCAUUAGGUAUAUUUCCCAAUGCUAUCC | |
| | CUCCCCCCUCCCCCCACCCCACAACAGUCCCCAGAGUGUGAUAUUCCCCU | |
| | UCCUGUGUCCAUGUGUUCUCAUUGUUCAAUUCCCACCUAUGAGUGAGAAU | |
| | AUGCGGUGUUUGGUUUUUUUGUCCUUGCGAUAGUUUACUGAGAAUGAUGA | |
| | UUUCCAAUUUCAUCCAUGUAUCUAAUUAAACUAAAGAGCUUCUGCACAGC | |
| | AAAAGAAACUACCAUCAGAGUGAACAGGCAACCUACAAAAUGGGAGAAAA | |
| | UUUUCGCAACCUACUCAUCUGACAAAUGGCUAAUAUCCACAAUCUACAAU | |
| | GAACUCAAACAAAUUUACAAGAAAAAAACAAACAACCCCAUCAAAAAGUG | |
| | GGUGAAGGACAUGAACAGACACUUCUCAAAAGAAGACAUUUAUGCAGCCA | |
| | AAAAACACAUGAAAAAAAUGCUCAGAAAAUUUUUAAGAAGAGAUACCAAG | |
| | UCUGGGGGAAAUUCUGGCAGAACCAACCUAACAGGAUUCCUGCUGAAAAC | |
| | AGGCGAGGGUGAUCAGCUAACAGGAGUGGGCAGAGAAAAAUUUUGAUCAG | |
| | AUACUGAGGGUGAUCAGAUAUCAAGGAUGAAUAAUUCUUGCUAAACUGUG | |
| | UUGGCCGUAUUAUUUGCUAAAACUGGGUUUUAGAAUGAUGUGCACAGACA | |
| | GGCCCAGGAGAAAGUCCAGAAGCCUGACUAAAGUUUGGCCAAGCAAAGAA | |
| | CUGUUGUUAGAAUACAGUCCCACAAGAAGCCAAAUGACAAAAUAAAUAUA | |
| | UAUAUAUAUAAACAACAGAAGAAGAGCUUCUGGCAAGUAAGUUUGAGAAA | |
| | UAGCUGUAUAUCACAUGCACCUUUGAAAGUGUAUUUUAAAUAACAUUAUA | |
| | CCAAAAUUUCUUCAUUUGUUCUUUCUGUAUCUACUCUAUUAAUAUGCUGC | |
| | UUUAUUCUUUUUCUCAUUUAUUAAACAUUAGGAUUUAAUAUACUAACUUC | |
| | UUUUCUAUUAUUAAUAACUGAUCAUCAUGACACUCAUGAUUAAUUAAAUG | |
| | UUUCAUUACAUAAGAAUGGCAUUGGGGCAAUAUGGAAAAAAGAGGUAGGU | |
| | UUUAUUUAUCUAUAGAUAUUUAUUUUAUUUUAUUUUUUUUUGAGUGAAAU | |
| | UCUGCUUUAUUUUAUAAUUAGAAAGGGCCAAGCUUUCUCCUGGUUCAGCA | |
| | AACCAUCUCUGAUGAGCAGGUUAGGGGCAGGUUACCAGACUACCCACCAU | |
| | UCCCUUUUGUCAGAUGCUCAGACUGCUCAACCGUAAGGGUUGGUCCUGCC | |
| | UCAGAACCCACUCUGGUCAUAGAGAUCACAAACUAGGGUCCAUCUGUCAG | |
| | UCCUGACAUUUCUCUAGGGGAAGGUGGAAGGAGAGAAUUAGAGAUGUCUC | |
| | UAGUUGCAGAAUUAUGGCCUGCAGUGGGUCAAAGGAACAUGACUAAGACC | |
| | UCACAAAAGACAAGAUUCUAUCUUGUGGUGAGGGGUGUGGUUGGGGGUAG | |
| | GAAUUAAUCAGAGUGAAGCAUUCUUGCCUAGAGGUAAAGCUCUCCUUUUU | |
| | CUUUUCUUUUUUUUUUUUUUUUUGUUUUCUUUCUUUUAUUUAAUUUAUGU | |
| | GAUGAAACCUGGUCAAAUUUUGGGCUGAGUUUUUUUUUUUUAGUUGAUCA | |
| | UUCUUGGGUGUUUCUGGCAGAGGGGGAUUUGGCAGGGUCAUAGGACAAUA | |
| | GUGGAGGGAAGGUCAGCAGAUAAACAAGUGAACAAAGGUCUCUGGUUUUC | |
| | CUAGGCAGAGGACCCUGCGGCCUUCCGCAGUGUUUGUGUCCCUGGAUACU | |
| | UGAGAUUAGGGAGUGGUGAUGACUCUUAACGAGCAUGCUGCCUUCAAGCA | |
| | UCUGUUUAACAAAGCACAUCUUGCACCGCCCUUAAUCCAUUUAACCCUGA | |
| | GUGGACACAGCACAUGUUUCAGAGAGCAUAGGGCUGGGGGCAAGGUCAUA | |
| | GAUCAACAGCAUCCCAAGGCAGAAGAAUCUUUCUUAGUACAGAACAAAAA | |
| | UGGAGUCUCCCAUGUCUACUUCUUUCUACACAGACACAGAAACAAUCUGA | |
| | UUUCUCUAUCCUUUCCCCACAUUUCCCCCUUUUCUAUUCCACAAAACCGC | |
| | CAUCGUCAUCAUGGCCCGUUCUCAAUGAGCUGCUGGGUACACCUCCCAGA | |
| | CGGGGUGGCAGCCGGGAAGAGGGGCUCCUCAGUUCCCAGCAGGGGCGGCU | |
| | GGCCGGGCGGGGGCUGCCCCCCACCUCCCUCCCAGACGGGGCGGCUGCUG | |
| | GGCAGAGACGCUCCUCACUUCCCAGACAGGGCGGCUGCCGGGCAGAGGGG | |
| | CUCCUCACUUAUCAGACGGGGUGGCUGCCGGGCGGAGGGGCUCCUCACUU | |
| | CUCAGAUUGGGCGGCUGCUGGGCGGAGGGGCUCCUCACUUCUCAGAUGAG | |
| | GUGGCCGGCAGAGAUGCUCCUCACCUCCCAGAUGGAGUCGUGGCCGGGCA | |
| | GAGGCGCUCCUCACAUCCCAGACGGGGCGGCAGGGCAGAGGCGCUCCCCA | |
| | CAUCUCAGACGAUGGGCGGCCGGGCAGAGACGCUCCUCGCUUCCUAGACG | |
| | GGAUGGCAGCCGGAAAGAGGCGCUCCUCACUUCCCAGACUGGGCAGCCGG | |
| | GCAGAGGGUCUCCUCACAUCCCAGACGAUGGGCGGCCAGGCAGAGACGCU | |
| | CCUCACUUCCCAGAUGGGGCGGCGGCCGGGCAGAGGCUGCAAUCUCGACA | |
| | CUUUGGGAGGCUAACGCAGGCGGCUGGGAGGUGGAGGUUGUAGCUAGCCG | |
| | AGAUCACGCCACUGCACUCCAGCCUGGGCAACAUUGAGCAAUGAGUGAAC | |
| | GAGACUCCGUCUGCAAUCCCGGCGCCUCGGGAGGCCGAGGCUGGCAGAUC | |
| | ACUCGCGGUUAGGAGCUGGAGACCAGCCCAGCCAACACAGCGAAACCUCG | |
| | UCUCCACCAAAAAAAUAUGAAAACCAGUCAGGCGUGGUGGCACGUGCCUG | |
| | CAAUGGCAGGCACUUGGCAGGCUGAGGCAGGAGAAUCAGGCAGGGAGGUU | |
| | GCAGUGAGCCGAGAUGGCAGCAGUACAGUCCAGCUUCGGCUCGGCAUUAG | |
| | AGGGAGACCGUGAGGAGAGGGAGACUGUGGGGAGAGGGAGAGGGAGAGGG | |
| | AGAGCAUCUAUAGAUAUUUCAAUUGUACUGGCUCUUGAUAUUUUUAGUAA | |
| | GAGUUUUCAUGUGAGGAAGUACUCAAGUUUGAAAAAACCUCUUUAUAAAU | |
| | CCAAAGACUUAGAAGGAGUUUCUAAUACAUAAUACAAAUGCCAACAAUUA | |
| | AGCAAUUCGAGGAAUUUUCUUACUGACCUUCCUAUUUAUUUAAAAUUAAU | |
| | AUAAAAAUUAGUCUUCCUGAUUUCUUCAUGGCUGUGCUUGAUUGCAUAAA | |
| | GUUGCUUUUACCUGGCCAUCUUUUAACAUUUUGUUUUUUAUUUGGAGUUG | |
| | CUAAAUUAAAGGAAGGAAGCUGUCACAAAUUGCUAAAAAUAAUACAUUGA | |
| | ACUAUAAAUAUGAAUCAUUUAGAUUCCAGUAUAUUUAUUUAUAUACCACC | |
| | CUUUUUCUGAAAUAAUUUGAAGAAGCUUAUUACUUUAUGUACAGAAUUUU | |
| | UUUUAUGAAGACCAUAAUGAGUACAGUGAACACAGCUGUAAUUACUAUUC | |
| | CAGCCCUGAAAGCCACUGUGAUGAAGUCAUGGAAUUAUGAUCUCUUCUAA | |
| | GCUUAUAAUUGUGAGCAAACAUAUGUACAAGUUUAUAUUACUGCAGUGGG | |
| | AUCCUCAGAAAGAAUCACAUUCUGGAAACAUUUAAGACAAAAUAAGCCUU | |
| | UCUGAUCUGCUAAUAAAGUAAACAAUCUUGAUUUAAAAAGAAUAAAUCAU | |
| | GUGUUAGGGACAAAAAUGUGAAGUUCUUUGUGUGAGAUCACAGCUCAGUG | |
| | CUCUCUUCAAAAACAAAAAGUCAUGCAGUUGUUUUUAUUGUUUAAAUAUA | |
| | AAUAAUUCACGUUCCAAAAAUGUCUCCUCAGGCUAACUCAGUAUCAUUUU | |
| | AAGGUAUUUUUUUUUAAGUUUACACUUGACCUGAAUCUUGUCUCUCAAGC | |
| | AGACAGGACAUGUUUUCUAGAAUAAUUGGCACAUUAAUAAUUAGCCAACC | |
| | CUGGGGCACAGGGAGGAAAGAAAACAUUUCAGGGCUAUUAGACACCAAAC | |
| | ACCACUUUCAAACGGGGCUGUACCUGGUGAGAAAGGGGGAUGGCCAUCCA | |
| | AGUGUCCACACUUGGAGAUUCUGCAAAGAGGCUUCCCCUUGCAAUGAUUC | |
| | UUUAUGCAGCCCAUAUUUUUCUGUGAGAAGUGGCAAGACAGAAAUGAAAG | |
| | CUGUGAAUUGAUCUGCCUGCUUAGUAAGCCAAGCUUAUAAAAGAAAAAAA | |
| | UUUGCUCAAGUUUCCUGACCUUGUCACCUUGUUUAAGAGACUGAAAGAAC | |
| | CUGACCUUUCCUUCUACCAUUUUGUCUGCUUCUGCAACUUGGCAUGAAUU | |
| | GUGAUUUGGAAUAGAGUUAAAAUCUCUUUUGGUGUUAGACUUAUUCAAGG | |
| | AAAAAUAUAUCCAAGGAUAUAUAAUUCUGAACUUCCAAGAAAAAAUCCUA | |
| | AAUGCAGCAAGUAAUGCAUUUUAAAGGAAACGUGAAAGUAUAAUUUGUGU | |
| | UAAAUUGUUAAAGGGUUUUAAAACAUUAAAAUUCAUUUAUUAAAAUAACU | |
| | UGCAUUUUACAUGUAUUGUAGAAAACCUGAAAACUCUAGAAAAAUAUAAA | |
| | GUAAAACAAAAGCAUCCAUACUUUUGUUAUCCAAAAAAUGCUAUUAAUCU | |
| | UUUAGUGUUUUUCCUAAAUAUAUAUAAUAACAGGCACUACAUUUAAACAU | |
| | AAUUUUUAUACAUUCAAUAGUUUUGUCUAUGGCCAUGAUAAUCUUAGAAU | |
| | AUCCGAAAGAGCCAGCAUAAUCUGUAAAGUUGCUGAGUGUCUAUAAUAAU | |
| | AUAGAAUAAAUGACAUCAAGAAAUAUAUUGGCUCAGGGGAUAUCUGCAUU | |
| | AUUUAAAACUCCUUCGAACAACCAUGUGAAUGAGCUUGGAAGCUGAUUCC | |
| | CCCUCAGUCCAGCCUUCAGAUGAGGCCUCAGCCUUGGCUAGCUGCUUGAC | |
| | UGUAACCUUAUGAAAGACCCCCAAGCUGGAGGCAUCCAGCUAAGCCACAU | |
| | CUGGAAUUCUCAACACACAGAAACAGUGACAUAAUGUUUGUCAUUUUCAA | |
| | CCACUGAGCUUUGAAAUAAUUUGUUAUGGAACAAUAGGUAAUUAGGUUAU | |
| | AGCAUACAAUAAUUAAAUGGACUUUGGUCAUUACACAAGCUAUGCCACUC | |
| | AGUUUACUACAAUGUUCAUCAAAUCACUUAACUUCUCUGGUUUGUAGUUU | |
| | GCUUACUUGUGGGAUUUGGCCAGAUUAGUGCUUCUCAGUCUUCAGUCAUU | |
| | UAUUUAUUAAUAUUUUCUAUACCUACUUGCAAAUUGUAGUGUUAUUUACU | |
| | GAAUUCUUCAAGUUUUAUUAUGAUGCAAAAAUUACUUGAAGCUAAUUGGG | |
| | CCAUUCAGCAAACAUGUCUUCAGUAAAUAUUGGUAGUCUUCCAGUAGUUA | |
| | UAUGUUGGUGAAGGAUAUUUUCCUCUUUCUGACAACUUGUUUUUAUCAGA | |
| | UGAUAUAUUACAGUGAGUGAAAGUCUCAGACUAAAUUCUGUCAAAUGUAU | |
| | GAACAUUUACUGUAUUUCACCAAAACUAAGAUAUCAUCUACUUCAAGAUA | |
| | CACCAUAAUAUUAUGGAACAGCAAGGGUGAAAGAAACACUGCAAAUCAUA | |
| | AUUAGAAAAUGUAAGACAUAUCCAAUUCCAGAUAUGUUAAAAUGUGAGAA | |
| | AAAAGUGUGUGCUUUAAGAUUAAAGAAAUAAGAUAUUAGCAACUAAGCAC | |
| | CAAAAUAUGUCAUCAUCCAUUCUAUUCAGUACUUUUGAAGUUACACUUUU | |
| | GACCUAAGGACACGAGCCCUAGCAAGAAAGUUUACUCCAAGAGGGGUAUC | |
| | CCACAGUUCUGAUGCCAGAAACAGCCGUCUUUGUAAUGAGAUCAGGCUAU | |
| | AAUGUUCUCCAUGCAAUGGAAGAAUGGGGGAAGAAUUGGCUCUCAUCUUA | |
| | AGUGCUCCUGAUCUUGUGUGCUACACUUGGCUUCUCAGGGUUUAUGCAAG | |
| | GGUGGUGGAGUCUUCACAGUAUUCCCAACUCACCUAUUUUAAAGAACACA | |
| | CAUUUAAGCCAAUAAAGAUUAUUUCUAACAUACCUUAUGCUAAUUAGCAA | |
| | AGCCUUUGGUACAGCUUUGAUUAUGGACAUGCUAUCUUUUAAGUGUAGUU | |
| | CUCCUUAGAUGUUGAGUCCUGAUUAGUACUUCAAAUAUUAAUUAAUCAUU | |
| | CCUGAUAUAUAGAUUAGAAACAAGUACUUCAAAAUACACAUCCUGCUGUU | |
| | UAUAAUGAAGCAUUUUAAACUAAAUGAUCACAUCUAGUAUAAAAACACUU | |
| | AUUUUUUCCAAAGUACACUUAUAUAGUUAGGAAACUACAAGUUACUACUG | |
| | UAAAUGGGAAGUGAGCAAUAGUGCUAUAAAUAUAAUAGAAGCCACCUGUA | |
| | AAAAGGAACACAUACAUCAAAACAAUGCUUGGUGCCAUUGCCUAGUUAAG | |
| | GUUCUAAACUUGAGGCCAGCUCUCCCUUUGUUAAAGAAGGUAAGUAAUUG | |
| | AGAUUUGUUUCCUUGAAUUAAUCAGAGGAAAAAAAAACAAGGCAGUCAUU | |
| | UUCUUGUAAUGUUACUCCAAAUUAUUUAAUGCUCUGUAAGUGUACCACCA | |
| | GGGUCUGGGUCCUGUACUUUGUGAAAAACUAGAUUACAUGAUUUCAAAGU | |
| | CUCUGACAGGUCUAAUAUUUUUUGAGAUACAAUUUUUCUUGUGCUUCUUU | |
| | UGUAAAGCUGUGGGUGGAAAUGCAUGUCAUUUCAGAAAAUAGUGCAUAGG | |
| | UCAUUGGCAUCUUACACAGAUUUGUUGCUGUGAAUCCUUGAACCAGGAAA | |
| | GUAUUUAUUUUUCUAAUUAGCUCAUUAAUAAAUGAGAACAUUAUAAACAA | |
| | UUGUGUAUUUUUUCCUCAUGGUGCUCAUAUUAUGCACUUAUAAUAUGAAA | |
| | GACUUUUAGGCAAUAUUAAAAUGUAGACUCAAGCCUUCUUGUUUUAGAAU | |
| | CAGUAACAACAAUACAACAACAAAUAACUUAUUUGUCUGACAGGCACCAG | |
| | GCUUUAAUAAGCAUUUUCCACCCCCGCAUUCUACCUGCCAGUGAUGAUAC | |
| | UGCAGCAUCUGCUAUGAUUUGAUUUUUGGUACCACUUUGUCUUUAUUUCU | |
| | GGAUAUAGAGCUUACAUUCUUCAUUGGAUAAGUACUUCCAAGUGGACCUU | |
| | ACAUAGCAAUCACAAUGUCAGAUAAGCUAAAAAAGCCUGUUCUGAUAACC | |
| | AAUAGUUUGGUUUUCUAUCAUCUUGGAAACAUUCUUUGGCAGGGUAUCUG | |
| | CAUUCUUUUUGACUUAUUGCAGAAGAGAACCAUUCUCUAAGUAACUCUUA | |
| | CAGGAAAAUGAGAAAAAAUGUUUUAUGACUUCCAAGAGUGAAGUAAGUUC | |
| | UGGAAUCAGUCACCUGUGAACAAAUGUUAGCCAUACAUUUCUAAUAGUGG | |
| | GAUAGGGAGUUCAGGGACAAAUGCUUGACCAUUUCUUAUGUUUUUCCUCA | |
| | UUUGUUUUAAAGCUUAUCCUCAACGACACGUUUUAUUAGCCUGACUAAAA | |
| | GUUUGAGAGGGAUACCUAUUUUAAUUCUUUUUUAAAAUUGUCUAGCAGUC | |
| | UAGCUGAAUUUCUUUUAUACAGUUUGACCAAGAACUAAGAAGCCCAAGUA | |
| | UCAGUAUCUCUAUUACAGAGUUUAUCAAUUGGUGUCUCCUGCCAAAAAUU | |
| | CUGAUCUCCAAUAGUCUUAUAUUUUAUUUUAAUAUUAACUUCAAUAUUUC | |
| | AGUUAACUUGAAAACUUCUGAUAUUUAAUAUCAAAACUAGUUUUUUUGCU | |
| | UAUCUUAAAUAUUAUAGUAAUCAUCCCACAAGUGGUUUUUUCUAGUACCC | |
| | UAACAACAUUAAUAACUAAAAGUGUGUUGUAUUAUAGACAAGAUUUAAAA | |
| | GCACAAUUACAACUGAAAGUAAAUUAGCAAUUACUUGUUGAAAGUGUCUU | |
| | UAAUGCUGUUCUGACUCCCUAGUAGCACUUAUUCACUUCUUUUUAUUGAU | |
| | UUUCUAUGCUUCCAAACUUUGCUUCCAUUUAUCAAUAUUUAAAUAUGCUA | |
| | UGUCUCACUCUCAUGGGGGCUAAUGUUGUAGUUUAACCAUCUGCAAGGUA | |
| | UUUCUUAUUGACAGCUAACAGCUACUAUUUUCCUCAAAGGCAACAAUGCU | |
| | AAUUAACAACUGUCUUUUAUCCAAUCCAGAGCAGUGACUGACAGUGACAG | |
| | UUUUGGCACUGAGGCAGGAAAGUGAAAAAAAGAUUUUUGUUAUAAUUUGU | |
| | AUUUUUUUUUUGGUAUGCGUCAAACCACACCAUGCAGAUAGCUAUGUAUG | |
| | GCAUAGAUUUCUAUCUAAAGGAGAGUAUAAUUUUCAUAUAGUUAUGCUUU | |
| | UGGUUCACUCUGCUAACAAAGCUUUUCCUUUCAAAAGAAUAGGUUUCUCA | |
| | CUGAGGUAUGGACAUGCCAAGUCCGAAAUUUAAAAUCUAAAACAUUCUGU | |
| | AUUAUCUGAGAACAAAUAAAGCUUUGGAAAAAUUUAUCUGAUGCAAAAUU | |
| | ACUAGACUGUUUUUUUCAUGGUGCCCAAAUAUAUUUUUCUUUAAACAUUU | |
| | AUCAACUAAUCAUUUUGUAGUUUUAGAAAAAUCAACAUUUAUUUAGAAGA | |
| | ACAUAUAGGCUAUUAAAACCAGUCAUGACAGGAAUGAUAAUCUUGGAAGG | |
| | UGAUAUGCAUAUCCAUUCCAGUGGUUUUAAUGCUGCCACAAAUGUUUGAU | |
| | UUUUAUUUAUUUAUUUAUUUAUUUUUUAUUUAAGUUAUAGGGUACAUGUG | |
| | CACAACAUGCAGGUUUGUUACAUAUGUAUACAUGUGCCACGUUGGUGUGC | |
| | UGCACCCAUUAACUCGUCAUUUACAUUAGGUAUAUCUCCUAAUGCUUUCC | |
| | CUCCCCCCUCCCCCUACCCCACGACAGGCCCCGGUGUGUGAUGUUCCCCU | |
| | UCCUGUGUCCAAGUGUUCUCAUUGUUCAAUUCCAACCUAUGAGUGAGAAU | |
| | AUGCGGUGUUUGGUUUUUUGUCCUUGCAAUAAUUUGCUGAGAAUGAUGGU | |
| | UUCCAGCUUCAUCCAUGUCCCUACAAAGGACAUGAACUCAUCCUUUUUUA | |
| | UAGCUGCAAAGUAUUCCAUGGUGUAUAUGUGCCACAUUUUCUUAAUCCAG | |
| | UCUAUCAUUGAUGGACAUUUGGGCUGGUUUCAAGUCUUUGCUAUUGUGAA | |
| | UAGUGCUGCAAUAAACAUAUGUGUGCAUGUGCCUUUAUAGCAGCAUGAUU | |
| | UAUAAUCCUUUGGGUAUAUACCCAGUAAUGGGAUGGCUGGGUCAAAUGGU | |
| | AUUUCUAGUUCUAGAUCCUUGAGGAAUUGCCACACUGACUUCCACAAUGG | |
| | UUGAAGUAGUUUACAGUCCCACCAGCAGUGUAAAAGUGUUCCUAUUUCUC | |
| | CACAUCCUCUCCAGCACCUGUUGUUUCCUGACUUUUUAAUGAUCGCCAUU | |
| | CUAACUAAUUAAAUGAAACAGCUUCUGCACAGCAAAAGAAACUACCAUCA | |
| | GAGUGAACAGGCAACCUACAGAAUGGGAGAAAAUUUUUGCAAUCUACUCA | |
| | UCUGACAAAGGGCUAAUAUCCAGAAUCAACAAAGAACUCAAACAAAUUUA | |
| | CAAGAAUAAAACAAACAACCCCAUCAAAAAGUGGGCAAAGGAUAUGAACA | |
| | GACACUUCUCAAAAGAAGACAUGUAUGCGUCCAACAGACACAUGAAAACA | |
| | UGCUCAUCAUCACUGGCCAUCAGAGAAAUGCAAAUCAAAACCACAGUGAG | |
| | AUACCAUUUCACAAAUGUUUGAUUUUUGCAAUCCAUGUGAUUUGUUUAGC | |
| | UCACAUUUUUUCUUGAUUCUUACAUUGAGAAAUGCAACGGGUAAACAUUG | |
| | AAACCUGUACAAAUGGUACCAGCUUGACAACUGCAUGGUUUUAUAUGCAA | |
| | AAACAAUGGAUUGUUGGGUAUUUAUAGAUUGCAUUCCAGGUAUGCAGUUA | |
| | CAUUUAUUAAUGCACAGUAUUUGUGUUUUUAACUAAAUGUUUUAAGUGGU | |
| | UUUUAACUUUUAUUUCACUUGUCCAGUGACUUCUGUUGGUUUUGAAGUAU | |
| | AACCAAUGAAUGUAUUCAGGAUGGAGGGGCCUUACUGAGGGUUAGAACAG | |
| | AUGCUGUAAAGAUCUAGUUAGCAACAUGGAUCCUACAGUCAAGCAACCUA | |
| | ACUCUGUCUCCACUAUUCUCUUAUGAGAGUCAUUUGACUUCAGCAGGCUU | |
| | AGCUUAUAAACUGGGAUUAUAAAAGACCUGACUGUGAUAAUCAGAUACGU | |
| | AAAGCAAAUAACACUGUGUCUGGCAUAUAGAAGGUGCACAACUAAUGCUA | |
| | AGUUUUACAUAGUUUUUAAUCUUAGGGAAUUAAAUUGUCCAGGAGAUGUU | |
| | CAGGAUUGGAGGGAUGGUGGUUAGUAUAGUGCUAGAUAAAAUUAAUGGUA | |
| | AUAAUUUGUUGUAUUGACAUGAAUUCUGUAUAUAUUUUCUUAGGCCGAUU | |
| | UACUCUUUCAGAAAUGUUUUAUUAUUUUAGCAAGACCGGAUUAAGGUAUA | |
| | GUGAUGCAGUGGAAAGAGCACUGGGCCUUAGAAUAAGGGGAUUUCCUUUC | |
| | UAGCCUUGGCUUUGCCAUUUAUUUUCUGGAAAUGAGAGUGGCCCAGUGAA | |
| | AGGUCCAUGGGGACAUUUGACAUAGGAUGGGACAACUGACCUAGUGAGCU | |
| | UAUCUGUUUACCUCUCUUUUUCACCUCUGUUGGUCUGUAUCUUUCAGUGU | |
| | CAGAAAAAUACAGGUAUAACUCUGCAACAAGGGACAUUUAAUUAGCAAGC | |
| | CUCCCUUGUUAGAACCUCGCUAAAUUAAUUAUUGAUCUUGUUUUAGUAUA | |
| | GAUUCUUCCCUGUUGCCAUGCCACAAUAAUUGCAGCAAAGUUGUGGUUAG | |
| | CUGGCUCUCGGACACUUUGGAACUGCUCUCCAGCUUUUAAAGUUGAAUAA | |
| | AUGACAUAUAAGAGCUUUCCACUCUCCUUAAGUUCCACUGUAUUUUUACU | |
| | CAGACACAACAGUGGCCAUUUGGGGCCAGAGAGCCACAAAUGCUCCUGAA | |
| | AUUAUACGGUUCUCCAAAAGCAAAAUAAAACAAAAUUUUAAAAUUAGCAA | |
| | ACCGUCUUCUUCCACCCACCCCCCAAACAAUUCUGAAAGCCAUGUUAAAA | |
| | UGUACCCUGUAAGCCUUCCUAUAAGCCUUAGUUCUUCAGUUGCCUUCCUU | |
| | CAGGCAAGAGAGGUGUCCCACAUGGGGUCUGAGCUUUAACACCACAGAUU | |
| | CCAUGAAACCUGUUCUUUGAUUAUCUCCUCUUUAAGGAAUGGGAAUAUGA | |
| | UGUUGGAUUCAGGGUACCUGUCUCACUCUUAUUGCCACAUCUCAAACACU | |
| | UUCUUCAUUUGAGUGACUCCCAGAGGCAGUCCUUUAAAUCUUGAUGCAUU | |
| | AUAUGCCAGCCUCCCUGGGCCUUAUGGGGGUCCCAAUUCACAUCUGGUUA | |
| | UCUUUGACCUUUAUCUUGGAAUUCUUCCUUCUUUUUAAUUCAUCCCACAC | |
| | UUCUUAAAAUGUUAAACGGCUAAUGUUGUAUGUUUCAUGAAAACAUUCUA | |
| | AUGGGACUGAUGAUAUUCACCUAUUAUUGGAGAAUGUGUCAUUUUUGUCA | |
| | GUGACCUUUUUAGGAGAGUGCCAGUGUGUUUGCUUUCUUUCUUUUUUUUU | |
| | UUUGUAUUUUGCAAUCCCGUGGUAUUAAUUUUUAAGUUUUAAAUUGAAGU | |
| | ACAUUAUACAAACCUACAAGAAGGGUACAGCUUAAUAAGGAAGAUCACAU | |
| | CCAUGUAAUCGCUACCCUGAUCAAGAGGUUGGCCACCUCUUUUAUCAAUU | |
| | AUCCACUUUUGACCAGAAAUAUAGUUAGAGUCUCACCUGGUUGAACAACU | |
| | GAAUCCAAAUUAUCUUGAAUAAUGAGAAUUUCUAGUGUUGUAACUUGAGG | |
| | CUUUGCCCAUGGCUUUGUUUUAUUAAACAUGUUUAUUGAUGACUUAGAUA | |
| | AGGCCAUGGAAGGCAAGCUUAUCAAACUUACGAUUGAGGCAGAGGGAAAA | |
| | AGGAAGUCUCCCUUUGCUUUCUAAUUGCAGAGAUUGUCCUCUUCUCCCUC | |
| | CACUUCACUAUUAUUCUUCUUUUUAGUUCAGGAAGUGGGGAGGCAGAGAA | |
| | AAUGAGAGAUAAUAUUUCUUCUAAUGGCAUUUUAGGGAUAGUCAACUAUU | |
| | UGCCUAUUAAAAGCUCGUUCUUCUUAACAGUCAUUUUGUGUCUUUUACUC | |
| | UUUUGUAUGAUUAUAAGAUUCUUUGUGAGGAUGAGCCAGUUUUAUAAAGA | |
| | UUCCCAGGGGGUACAGCAUUGGCGAUUCAUUCUAAGAGAAAAUAAAUGAA | |
| | AUUGACUUGGGAGAGUAUUACAUUUUUUCCUAAUUUGAGAAAACUCCCAG | |
| | AAGCAGUUCUGUUUCCUAAUACCUGGAUUCCACAUUUAUGGUAAAUUUAG | |
| | CAUCUUUCUGGCCCUGCUUGUCCUUAUUCCCCUGCUUUUAAUGUCUCCCA | |
| | UGCAGAUGACACUCCCAAAUAGAUGUUCUGGGUCUGCACUGAAAUUUCUA | |
| | CCUAGCAGCUCUUGGCUGCUGCAGUUGCUUGUUUCUCAGCAAAAGUUUCU | |
| | CCCGGAGUAGAGGAAGGCUGUAUUCCAUCACCUGGGAAGGUCUUUUCAUC | |
| | ACCUUUGCUCUUGUUAUUUUUUGUUCAGUUCCAAAAUAAUUUAUUUCUGU | |
| | UGCUGCCACCACUCUGACUACCUCCGAUAUCAGAGAAAAGGACCCAUGUC | |
| | UUCUAGAAGACAUAAGAUUUUAUUUCUCUUGAUAUGCAGAAAUCAGAGAC | |
| | CAUAUGUUAUUAAUUUUUAAAUUCCAAUUCCACUAAUAGUAAUUAAUACA | |
| | UGGUGAAUAAAUAAAUAGAAGUCCCAAUGUUGCUGAAUACACAUUUUAGA | |
| | CACUAUCCAAUUCUCUAAUAAAUAAGAAGAAAUAAGACUUUUACUAAUUC | |
| | AUUCAUUUCUUUUUAUGUUCAUUUUAUAAAUUCCACUUAUAGUUAUCUUC | |
| | ACUAAUAGAUGAGAAUAGAAACCAUAAAUUCAUUAAUAUGCAACUGACCA | |
| | GCCCAUUUCACUUGGAAUGCAUUUUGAGUUUACAGGCAGAUUCAUAUAUA | |
| | UCUAAAGCCUAAGCACUACUAAUAAAUUAAAAAGCCAGGAUAUCUACCAA | |
| | UUUAGGAGGAACUGGGAAUAUGUCCAGUUUUUUUCUUCUAUUUUUCUUUC | |
| | UCUCUCUCUCUGCACACUAUACAUGGGCAUCUUGGUCAGUCUUAUUCUAA | |
| | AGCAAAGAAUGAGCAGAAGUGCCCUCUCAGACCUUUCCUGGAAAAAUAGU | |
| | CAUUUAAAUGUCUGGAGUUAAAGGCCUUCUUUGUAUAGUAUACAGAAAGU | |
| | AAUUAGACUCAAAACCCUUUAGACAAUCAAUGCAGGUAUUAAAUGCAUUA | |
| | CAUAUCAGGAGUAUUAGGGAGAUAAAAAUAGAGCCAAAUCUUAUGAUGGA | |
| | GGAGGCCAACAAUCUCCCAUAGAAAAAAACUACAUGCCAGACAGUAGAUU | |
| | UUUUUGCCAAGACAGUCUCCUAUGUGGCACAGACUAUGAAAGCUAUGGUC | |
| | CUUCAGAGGAAGUAUAGGUAAUUUAACCAGGGAGUAAAGAUGAGUGACUA | |
| | GUGGAUGGGGAACCACUGAAAUUAAAAAAAUAUAUAUUUAUAAGACAACU | |
| | AGAGAGCUACUGUCAGAGAAACCAGCUAGAAAGUACAUUCAGUAAGAGAA | |
| | AAAUGUGUGGUAAAAAGCCCUUGAAUAGACUCAUAAUCAGGGCUGGCUUC | |
| | AUGGGCCUGAGACCUAUGGAGUCACACAAGAUUCCACACUAGAAGGGCCC | |
| | CAUACUUCAUUUAAUACUCUGCUGUAGCUAUCUUUAAAUUCUGAAUAAUU | |
| | UCUAAAUAGAAAACUCAACAUUUUCAUUUUAUACUGGGCCCUACAAAUUA | |
| | AGUAGCUUGUUCUGCUGGUCAUAUGGAAAUUAAUGAAGGUGACAUGAAAC | |
| | AAGAGCAUGAGAAAGGAUGAAGGGUUGUGGCUGUAUGACCAGAGAGUAAA | |
| | AAUCUAUAGGGUGGUAAAUUAGAAUUCCCACAUUACAGACAAUUCAGGAA | |
| | GAAUAGGAUUGAGGACUACUCUGAGCAGGCAAUCUUUUUGGAACUAUUGU | |
| | GUUGGAGUAUUGAAGGCCAGAGGCAUGUAACAGGGUCCAAAUAUGCAUGA | |
| | AAAAAAAUGGCGAAGACCAAGUCUCAAGUUAGAAGCUAGUUAGCCUAGGA | |
| | AGGCAUGCAUUCCAGGUAUUGGGACAGAGAAAAAUGGGCAGCCUAAAAUC | |
| | AAGUCACAGGACAAGUGGUUUGUAAGAGGAGAAGAAAGAAAGAAGGCUGA | |
| | AGAGGCCUCAGCAAUGGGCAUAAUGCCAAGCCUAGUGACUGCGGUCAUUG | |
| | UGUUUCACAGAAAUUUGUAAGAAAAUCUACCUUUUGUGGAAAGAUGAUGA | |
| | ACAGUUUUUAGUUUGCAUGGUGGUGGUGUUCAAGCAAAAAAUGUCCAAGA | |
| | UGUAAUUGGAGAAAAGAAUAUGGAGCUGCUAGAAGAGCUCAGGCACAGAG | |
| | ACAGAUUUAACAGUGAGAGCUAACAUUUGUUGGAGGCUCACCUUGCACCA | |
| | CACACCAUGUCGCAUUCUUCUCUUGUGGUAGAAAAAACACCAGAUGGUAU | |
| | AUAACAUCCUUUCAGAGGAAAAGGUAAUGUCUUAUAAGAGGAGAAGGGCU | |
| | UGUGAAAGCUAGACUUACAAGAAACCUUGUUAUUAAAAUUUGGGUCUAUA | |
| | UUUAAGCCCCUUCCUUCACUCACUCAAAAAAUACUUACUCAAGAUAUUAC | |
| | UACUCUUGUGUUUAAGAUGUUCAUUUUAGCUAUUGCUUUUUAUGUGUGAG | |
| | AGUUGAGAUAUCAAGAUGACAGAGAAACAAGAUACAGUGAGGUUGCCAUU | |
| | AUAAACUUUAGAAAUAAAUAAAAGAAAUUAGCCCCAUAAGUUUCAGAAAC | |
| | UCAAAUUAAGCUUAAUGACAGAUAGAUUAGUCAAGGUAUACUAGGGGGUA | |
| | GUAGAUAUUUUUCAUUUCUGCACAAUCAAAAAUGUUGAACAUCUUCCCUA | |
| | UGUGCUAGUAAUGUAUGAGGUCCAAAUUCUUAAGGUAGAAAACCAGAAAA | |
| | UUAGAAAUUUCAGCCUCUGGUAAGCUAGGAGUGACCAUGUGCUCUGGUGG | |
| | GAGUCCCUGGGAAGACCCAUUCUCUCAAAUAAAAUUUCAAAAAGAGAAGG | |
| | GUUUUACACUUUGCCCUUUAGCUAUUCUUUAUUCUGUUCUGCCUGAAAAU | |
| | CAGAUAUAUUGCCUUUCAGGGUCUCACCAAACCUAAGAGGUGCAACAUUC | |
| | UCUUUAUUAUGACAAAUGCUCUUCUAUGCUAAGUGUGGUAGAACAGGGAA | |
| | GAUGGAGGUAACUUAGGUCCUAGAUGGCAUCCUUACUUCAGCUCUGGACA | |
| | UCACGGAAUUUGAAAAAAAGAAAAAGCAAACAAACAAACAAAAAAUGAAG | |
| | CUGCUGUUGUUUGAGUUUUCUGUUCUUUGUAUCCCAAUGAAUAAUUUCUG | |
| | GUUAAUCAAUGGGGCAUAUCACAGAAUCUGAGGGCAAUUAUGUGAUUAGU | |
| | UCUCAAGAGCAUCCAGGACUCCAGUGAAAUCAGGACUCUGUCUCUCCAUU | |
| | UCCACUUUUCUUUCUACUUUGGCUUCAUUCUUUAGUUGCAGUAGAGUAGC | |
| | UUUUGUGGUUGAAAACAUGGCAACUCACACCUCCCAGUUUUAUGUAUUAG | |
| | AGCUCCUGCCACCAGAUGACUUCUUUGAUUUCAGGUCUCCAAAUCCAGAA | |
| | AAAUGGUUUCCUUGUCAUGGCUUAGGUCAGAUUUCAUUUCAGUAUCAAUC | |
| | AAUUUUGGCCAGAAGGUUGAAGUCUUUUAAGAAUUUAAUCAUUCCCAGAA | |
| | AAAGUAUAUAUAGAUGGAGAAAGGGCUGAGGAAGCUCUCAAAAGAAGGCA | |
| | UUGGAACUAGGGGCAUUCUGGGUACAUAAAACAAUAGCCCUCAAUUAUGA | |
| | UUUGUAUUACACAGGAGCAAACUGUAUUGUGAGUCUGAUUGCCCACUAUG | |
| | AACUACACAUUUUACACAGUUAAGAGAAAGAGUAAUCCUCUCAUCUUUUU | |
| | UUUUAAUUUUUAAUUUUUGUGGCUUAUUUUUAGUACCCAGCCUUAAUAGA | |
| | GUUGAGCAAAAUAGAGAUUACGUUUCCAUGACAUAAUAAAUUUUGGUAAU | |
| | UUCAGAAAGCAAAAAAUACUGGAAUCUUUUCAAUCCAAGUCGAUUGGAUA | |
| | CAUAGCAGUUCAAUUUGAACACAGCAGUUCAAUUUAUUUGUUAACAAAUU | |
| | CUGCAGUGUCUUCCUGUAAGAAUCUGUAAAAAUCCUCAUCAGCAUUUGAA | |
| | GAUGCACCCCCUCACUUUUGUAAUUGGUUAACAGAAGCUUCUAUCUUUUC | |
| | ACAUCCCUUUAGUGAUCUCACUGUUUACUCUUCAAGCUGUAUAGUGGCAG | |
| | CUGUUUAUAUGCUUGCAUACACAUGGUUUAUUGGAAUGUUCUCUUCAAAU | |
| | UGAAAUUAUUUAUGUUUCAAAAUUUAUAUACACAUACUAUCUAUGAAAAA | |
| | UCCUGCACAAUUGUGUAUUUUUUUUGCUGUGGAUACUGCAUAAAUAAAAU | |
| | UUAAAAUAAGAAAGAUGUUUCUCUUCUUUACAUUAUUACUCUAUCUAAGC | |
| | CUACUGAUAUUAGUUAAACAUUGUUAUUUUCUGGCAACGAUGUCUUUGAU | |
| | GUGUGACACAUUCUUGCUGGAAUCCAGCCUGUGGCUGAUUGUAUUUUCUA | |
| | GAAAGGGCUGCACUAAUACAUAGCCCAUCCCACCCACAUGCUCUUCUUGC | |
| | AAUGUGACAACAAUAAAUGACAUUGUCUAACCUAUGUUGGGAAUGAUAGU | |
| | CUCUGUCUUUGAAUCUGGUUGGAGUUUUGUAAAUGCUUGGACCAAUAGAA | |
| | UGUGACAGAAAUAAUGCUGUGUGCCUUCUGAGACUACAUCAUAAAAGAUG | |
| | AUGUGGCUUCUUCCUUGGUCUUUCUUGGGAUGUUUACCUUUGGGAUCUGG | |
| | CUACUAAUUUGUGGGGAAGGCUAGGCAACAUGGAGCGGCCAUGUAUAGCU | |
| | CUUCCAGGCUACAGCUGCAGCUAAAUUCUCAGCUGACAGCCAACAUCAUG | |
| | UGUCGGACAUGUGAGUAAAUGAUCUCAUUUUAUUCCAGACCUCAGCCUUC | |
| | AAGUUUUCUAGCUGAGGCUAGGUUAUUUCGUGCCACUUGCUCUGGGGGUA | |
| | UUUUUUUUUUCUCUUAAUACAACCAUAAUAAUGGAACAGAGUCCAUUCUG | |
| | UUUCUGAAAGACACUUAAGUGUAAAAAUAGUAAAAAUAAAUUAUCUCUUU | |
| | CUAAUAACAUUACAUCCUAUAUAAAAUAGAAAGCCUUUAUUCAGAUUACA | |
| | AUGAGCUAUUCUAAAGGGGAAUGUUUAGAUAUAGCUUUGGCUUGCUAACU | |
| | AUUAAAUUAUAAAAAAUUAUCUCAGCGAUCAUUUUCUAUGCAUUUAGCAU | |
| | GUCAGAGUUCAAAUAAUCUAAGAAAAAUACAUAGGAGAAACAAGACCUGA | |
| | GGAGAUGUUCCUGUGAUGGAACACUGAAACAGGAUACUGAUGGAGACAGG | |
| | GUUCUCUAAAAUUAUUUGAAAAUAAACCAGGUUUAAUAUCCUGCAUAGAA | |
| | AAAAGAGCCAUAAAAUAUGGCUUCUAUCACUGUACUACACUGUGUUUUUU | |
| | AAUUUUUUAGAAGAGUUAUUUAAAUUGCUAAUUUAAGACUUUAAAAUUUA | |
| | UAAAAUUUUAAAUUUUAUUUGUGAGGUGUUUAGUUUGCUUUCUGAAGAAU | |
| | UUCUUAAUUAGCAUGUCAUGCUAUGUAGGACACUUUAGUAUCCCUGAGAC | |
| | UGAAGCAGAUUUUCACAUUUGGAUUCUUAUCUAGCCAUAAUUUUUUGGAU | |
| | UGUUUUCAUAUUAAGCUUUACAUUUUCGAAUAUUUGUUGCUGCAUAUGUC | |
| | AAAUAAUUAGAGUCUUUUACUACCUUUGAGACUCUAAUACAAAUGAAGAA | |
| | UCUAAACAAGCAAUUAAACAAUAGCAGGAUAUUCAGCUGGAAACAAUUAA | |
| | GUACUGCCGAUUAGAAGCUCAAUCCUCUAGAAUAAACUCUUACACUAAGA | |
| | GCAAAGUUUAUAGCCACUCUACAAUUUAUUUAACACUUCCCAUCUCCUUC | |
| | AUUUAUAAACCACAUAUGUAUAUUUUCUUUCUUUCUUUUUUGAGACUGUC | |
| | UCACUCUGUAGCUCAAGCUGGAGGGCAGUGGUGUGAUCACGGCUCGCUUC | |
| | AGGCUCAACCUCCCAGGCUCCUGAUCCUCCUGCUUCAGCCUCUUGAGUAG | |
| | CUGUGACUACAAGUGUGCACCACCAUGCCUGCCUAAUUUUUUAUUGUUAU | |
| | UUUUGUAGAGACAGGGUCUUGCUAUGUUGCCCAGGUUGAUCUCGAACUCC | |
| | UGGGCUCAAGCAAUCCUCCUGCCUCAGCCUCUCAAAGUGCUGGGAUUACC | |
| | UGCAUGAGUCACUGUGCCCAGCCCCAUAUAUUUUCUUGGUAAAUGUGCAA | |
| | UACUCAGGUUUCCUUUCUGUCUAUCAGCCACUGGAUGCUUGAUUUAGUUA | |
| | UACAGAGAAUUGCACUGGCAAGCUGUUCAAAACCUCUUCUUAACUUUUUU | |
| | AAAGCAAAGGCAAACAGAAAAAGUAGGCGAGACAAGGGGAAAAAGAAAAA | |
| | AAAUCUCUCAGGAAAACUUCUGAAGGUAAAAACCACUCUGGGUCACAGGC | |
| | UUUGGUCACAACAGCUCAGUUAGGGACUUGUUCUUUCUUUCUAUCCCUUC | |
| | ACACACCAGGGGCAACAUUCUUCCUGGGACUUACUAUGCGCCACAAGAAA | |
| | CUCAUUAUUCGCCUUCACCAAAAUUUAAACUCUUCCCCUUUCUGCAUUUC | |
| | CUGCUUCAAUUAUAACUCAACCUACUAAUCAAAUGGCCAAACUAGGGAUA | |
| | AUCUUAGGCCCUUCCUUUUUCUCCACUAUCAAUCUAAUUUCUUUCCAAAU | |
| | CCUGCUGACCUAUCUCUGUAAUCGUUUAUCAAAUCUGCUUCUUCCCAUCU | |
| | CUGCUUGCCACUGCCCUAGCCUGAUCUAGAUACUUGUAGCAUUUGAUUGG | |
| | CUAUGUUACUGGUCCUGCUCUCUUAGAUAUGUCUCUACUCUGACAUCAGA | |
| | GGAAUGUAUAUAAAAUGCAAAUCAGAUGCUGUCAACCCACUGCUUAAAAA | |
| | UCAUUCAAAGCAUUCCAUUGCCUACAAGAUAAAGUAAAAUCUAUUUACGU | |
| | GACAUGCUGGAAUUUCUGUAGCCCUGUUUGUCUCUCUGUGUCCAUCUUUU | |
| | AAGUAUCCUUUUGGCUCUUCUUUUUCCUUUUCUUAACUUAGUAUCCUACA | |
| | UGGAUGCUGAAUUUCCUGUGACUCCCUCAAGCACCUUGAGUUUUCUGUCU | |
| | UUGUGCAUUUCUUUGUUUUCAUUCCAGCUCAGGCAACACCUGUUUCAGGA | |
| | AUGCUUCACUUCUAUUCUCUGCCAAAAUUUUCACUGACCAUUUCUUUCAU | |
| | AGUGCUUACUUUAUUAUAAUGAUAUUUUGUAUUCUUUAUAUUUUGUUUAG | |
| | AUGUUGUCUUCCUGAUUAGGAAUGUCCUUAAUUUUUGGAGCCAUGACAAA | |
| | UAUUUUUAAAUGAUGCCAUGGCUUUGUUUGUAUUGUGAUUUACAUAAGAU | |
| | UUCUCAUUUACAUAGUUAUCAAUGAUGUAACUAAAUCCAUGCUGAUAUGC | |
| | UUUAUAAACAACCUGUUAGAAUCCAUCAUGACUCAAACCAAAGUUGAGCC | |
| | CAAUUUCAUGGAAUUUUCUUCACCUUUUUUAUAGGAAACUUUUGUCUCCC | |
| | UUUCUUAUUUCUGUGAGAACCAUGGCAAAUUGUAAAUCUUUAUACAAUCA | |
| | GUACAAAUACUACACAGUCCAUAACUAUUUGUUGACCUGAAGUAAACCAG | |
| | AUUAAUAAAGCAUACACUCUGGAUUGAAUAAUCAUAAAGUCCUUAACCAA | |
| | ACUCAUUUCCUGAGCCUGGUUAUUAGAAUCUAAGCAUCCAAUAAAGGCUU | |
| | GCUUCAUCUAACUGAACAGGCAUAGAUUAAUUCUUUAUUUCUUAAAAAUA | |
| | AACAAAAAUGCAUUUUAUUUUUAGCAAGAUAGUUUUCACCAAUUGGGCUA | |
| | GAACAAUUUUUGAAAGUGAAGAUAAGAACUUCAAAGCCCCCACAGCCUCU | |
| | ACCAUCUGCAUUAGUUUCCUAUUGCUGUUGUAACAGCUAAACAUUUAGUA | |
| | GCUUAAAACUCCACAGAUUUAUUAUCAUAUAGUUCUGGAGGUCAGAAGUC | |
| | UGAAAUGGGUCCACAGGGCUACAUUUCUUCUGGAGGCUGUAAGGGAGAAU | |
| | UGCUUCUUGACUUUUCUAGCUUCUGGCUGCUGCCUUACUCUUUGGCUUGU | |
| | GGACAGGGAGAUGCAUCCUCACAUCUAUUCUGAUUCUGACUUUCCCAACU | |
| | CCCUCUUUUCUCUAUUAGGGACCCUUGUGAUUAUGUUUGGCUCACCUAGA | |
| | UAAUGCAGUAAUCCUCCUAUCUUGUUAUCUUUAACUGAAUCACAUCAGCA | |
| | AAACGUCUCCUUUGCCAUAUAAAGUAACAUAUUCACAGGUUCUGGGGAUU | |
| | GGAUUUCUUUGGGAAGCCAUUAUUCUACCUACCAUACCAUCCAUUUAUAU | |
| | UGCUAUGCAUGACAUCUUUUGUUUUUCAACUGAGAAACAACUGUACAUAU | |
| | GGGUUUGGAUGAGAAACUGAGCGAUUGAGAAAAAGUAUAGGAAAUGGAGU | |
| | AGAUUAUUGUAUUCCAUUAAACCCAGAGGGUAUUUAAGCUCCUAAAUUUG | |
| | GGAAAGAAUGGUCCUUAACUGGAGGAUAUUAAUUGUUGUUGAUUUGUUUU | |
| | GAAACUGGGUAAUUUAUAAAAAAUGAAAUUUAUUUCUUACAGUUCUGGAA | |
| | UCUGGGAAGUCCAAGGUUGAGAGCGUUUAUCUGGCAAGAGCUUUCUUGUU | |
| | GGUGGGGAUCCUCUGCAGAAUCCUGAGGCAGCACAGGACAUUACAUGGUG | |
| | AGGUGGGGCUAGAUGUGCUAGCUCAGAUCCUUCUUUCUCUUCUUAUAAAG | |
| | CCACCAAUCCUAUUUCCAUGAUAAAUUUUAAACGAGUUUGAAGGGGACAA | |
| | GUAUUCAAAUCAUAGCAUUCUGUCCCUGCCCCACCCCCAAGCCUAUGCCC | |
| | UGCUCACAUGCCACUCAGAAUUAUUCCAGUGAGGGCUCUCUGCAGUGGCU | |
| | UCCUCCUGAAACAAGUCUCUGCCUGGGCCACUAGGAGUUCAACAACAUCC | |
| | UUUGAAAUCUGGAUGGAGCUGCCAAAUCUUCACAGCUCUUUCUUUCUGCA | |
| | AGCUUGCAGAAUUAGCACCACAUGGAUGUUACCAAGGUUUACAAUUUAUA | |
| | CCUUCUGAAGCUGAAGCAUAAGGCAUACAUGGGACUUAUUUAGCCACAGC | |
| | UGGGGUAGCUGUGAAGCACUGCACUGUGGUACAGGAUUAGAAUCCCAAGG | |
| | UGGCCCUGGGCAGUGAUCUCAUGGAGAAUGUGAUGUCCAGUCUCUGGAAA | |
| | UUAUUUUGCUCUCCUAGGCCUCUUGACCUGUGAUGAAAGGGGCAACCUCA | |
| | AAGUUUUCUGAAAUGUCUUUGAGGUCUUUCUCCCAUUGUCUUGAGGUAGA | |
| | GCACCUAGCUCCCUUCUAUUCAUGCUAAUCUCUUUGGCAAAUGGUUGCUG | |
| | GGCUUGGUUUCUUCUCCUGAACAUACUUCUCUAUACUUUAAGUGUCAGAC | |
| | UGAGAGUUUUCCAAAGAAUUCUACUCUUUUUCCUUUUGGAUUAUAAAUUC | |
| | GGUCCUUAAAUUAUUCUUUUGCUCCCAAAUCUCAGCACAAGUGGUGAAAA | |
| | GUAACCAGGAAGCUUCUGUAUUUUCCUUAGAAAUUUCUUCCGCCAGAUAC | |
| | CCUAGUUUAUCAUUCUCAAGUUUGGCUUUCCACAAAACCUUCAGGCAUGG | |
| | AAACAACUCAGCCAAGUUCUUUGACAGUUUAUAACAAGGAUGGCCUUUAC | |
| | CCCAGAUUACAAUACGUUGUUCCUCAGUUCCAUCAAAGACCUCAUCAGAA | |
| | UGGCCUCUACUGUACAUAUUUCUAUCAGAAUUCUGGUCAUGACCACUUAA | |
| | CCAAGCUUAAGGUGUUCCAGACUUUCACUAAUCUUCUUGUCUUCUAUGUU | |
| | CUCACCAGAAUCUGUUUUUUUUUUUUUUUCUAGCCUGAUCCUUUAAAUUC | |
| | UUCUAGCCUCUCUCCAUUGCCCAGUUCCAAAGCUGCUUCCACAUUUUCAG | |
| | AUAUUCAUUCUUGGCAACAUCCACUUCUGAUACUAAUCUUCUGUCUUAGU | |
| | UUGUUCAUCGUUGCUUAUAACAGAAUACUUGAAACUGAAUAGUUUAUAAG | |
| | AAGUGAAAUUUAUUUAUUGUAGUUCUGAAGGCUGGGAAGUCCAAGGUUGA | |
| | GGGGGUGCAUCUGAUAAGAGCCUUCUUGCUGGUGGAGACCUGCUGUAGAG | |
| | UCCCAAAGUAGCAUAGAGUAUCACAUGGUGAGGGGGCUUAGUGUGCUAUC | |
| | UCAGGUCUCUCUUACACUUUUUAUAAAGCCACCAGUCCCACUUCUAGGAU | |
| | AAAUUUCAAAAUGAGUUUUGGAGGACACAUAUUAAAGUCAUAGCACUUGC | |
| | CUAUGAGGUCAUGAAAGGUCUGAAAUCAUCCUGCCUCUCCAGCCUCAUCU | |
| | UGUGUCCCUUCCCUCUUGAUUUUCAAGCUCUAUUAACUACACUGUCUCUG | |
| | UUCACAGAGAUUUAUAUAUCAAGUAUCGUUUUUCUGGAGUGCUUUUCAUU | |
| | UUAUAAUUCAGCUCAUUAACUCCUUCUGAUAUAUCACAUGGUUCAUUGAU | |
| | ACUUUGUUUUGACACAUUUCAAUUUGAAAUGUUAUGCUCACUUGUUGUAA | |
| | CUACUUUUUAACACGUGUUCAUUUAAAGAAAGAAUAACACAAGAGUCCCA | |
| | CUAUAGAAACUUCUAUUUAGUAUUGUACUGGAGAUUCUAGGAGAAAUUUU | |
| | UCUAUAAGGAUUAAAAACUCCAAACUCAUGUUGAAAUUUAUCAUAGGAGU | |
| | GGGACUGGUGGCUUUAUAAAAAGAGGAAUAGAGAUCUGAGCUAGCACACU | |
| | CAGCCCCUUCACCAAUGUGAUGCCCUAGGGCUACCAUGGGACUCUGUAGA | |
| | GGGUUCUCACCAGAAAGAAGGCUCUUACCAGAUUCAGCCUCUUCACCUUG | |
| | GACUUUCCAGCUUCCAGAACUAAAACAACAACAACAACAACAACAACAAC | |
| | GAAACCUAUGGAAAACCCAAGAGAAUUAUCAGACGAACUACUGCAAUUAA | |
| | UUAGAAAGUUUAGCAAGGUUGUGAGAAAUAAAAUCAAUACAUAAAAGCAA | |
| | AUUGCACUUUAUGUGCAGUUAGAUAAUUCAAUUUUAAAAGACCAUUUUUA | |
| | UGAUAACCAAAAAAUAUAAGCUUUAAUAAGUAAAUACUUAUUAGUAAGAA | |
| | UACUUAAAUUAAGAAUUUUGUUCAUCGAAAGAUACCAGUAACAUAGAGAA | |
| | GUAAAGCAAUUUUUAAAGACAGAACAUUGGAAAAAAGCCUUAGCAACCUA | |
| | CUCUCAAGUCUCAAGUACACAUCUAACAAACAGUCUGUUUACUUACAAAA | |
| | AUGUCUGAGUCUGUAUUUCCUUCUCUGAAAAAUAGGAUUUGAAAUAGACA | |
| | AUCUUGCAGUCCUCCAGCUGAAGUUCUUGGUUGUUAUAAGAUAUUCACUC | |
| | GUUGCAAACUUGUCUGUUUUGAUUUUUGAAAGGCAGACUAAGAUUCUUCU | |
| | UUAGAAGGACACAUUAUAUUCUGAUUAUUCUCUAUGAAUAUAAUGAAUUU | |
| | GAAGACCUCCAGGCAAUUUAUCAUAUUAGCAUAUAUGGACCUGUCUUGUU | |
| | UACUUAUAUCUUACUCAAUAUUUUCACAUUGGACCAUAAGUCAUUCACUG | |
| | UAACUUGCUACUUUCUGAUUCUGAUUUUUCCUGCAGCUUAAGAGGAAAUU | |
| | UAAAUAUAUAUAUUUAUUCUUACCUUGGAUUUAUAGUGAUGCUAAAUGGA | |
| | GGUAGUUUGAGAGGGGUGAAUUUAAGAAUGUGGCUAAAGAGUAUAGUGGG | |
| | AGGUUUAUUUCUGACACUAGAGGUAUUAUGGGAGGGGCUACCUAUGAUUA | |
| | UAAAAGAAAUAAAAUAUACAAAGCCCAAGAGAUAGUUAUCACAUUGAAGA | |
| | AUUUCUGCUUUACCAGCAAACUACUUUCUAUAACUGUCUCCAAGUCAUAA | |
| | AGCUUUGUUUAUGUGUAAUAUCUGAAAAACAAACUCUAUAUCAUGAAGCG | |
| | GUUUAACAUGCUGUUUGGUCUUUGUGAAGCAAAUUUGGAGUUUAGCUACA | |
| | AAAUUAUUGGAGGAUACUGUAAUUUAUAGUAACAAAAGCAUUUUAUCCUG | |
| | UGAUUUCAGGAAACCAAGAAAUUAUUUUGCAUACCACACUGCCACCCUGA | |
| | AUACAAAAUUUAAAAGUUAGCUGUAUGUUUAUGAAAUAUAAUACAUAUGA | |
| | CAAAGCAUAUCCUUCCUUAUUCAUCUCUGAAAUCUAUUAAAGUACAUAGA | |
| | AUAAAUGUAGAAAAGUAACAGCAAGUUGGAAAUAUAAUUUAGGUUUGCAG | |
| | ACAAUAAGUAAAUAUUUGAAUAAUUUUGAUAAAACAGGCCACGAUUUUAU | |
| | AGGUGCUUCUGUCAAGUUACUAAAUCAAAAUAUUAUGGAAUUCUUUCAGU | |
| | UAUUAAAAUUGCAUUCGAAUUUUUUAGAUGAAAAAAAGCAAGGUGACAGG | |
| | CUAUACAUCAGUUAAAUUGUGAGCAUUCUUUUCUAAUUUUCCCCAUAAAC | |
| | CAGAAAUAUUAAGUGUCAUGCUAACAUCUGUGGCCUGAACAUAAAGUUGA | |
| | CAAAAAUCAAACCUAAUGGCUUUUCCCACUGUAAAAUAAUGUCUAGUUUC | |
| | AAUCUGCAGUUUUUUUUUAAGUCUCAAGUGUCAAAGUAAUGUCUCAUGUU | |
| | UGUAACCACUUUGUGUUUUCACAUCAAAUAUAGUUUCACUAGAAUCUAAG | |
| | UUCUUUUUAAAAAGAAUUGGUUUAUGAUGACAUUUUGUUCUCAUAUGGUC | |
| | UCAAAUAUUCUUUUUGUCAAAUAAUCAGAAUGAUGAGGGUACUAGUAAUC | |
| | AUCUAAUAUAACAUUUUAGUUUUUGACAUGUAGAGAGAGAGAGCCCUUUU | |
| | UAUUCCAUAGGAUCUAAUUCAGUAGAAAAUGAAAACAAAUUCUCCAAAAG | |
| | UGGGUUCAAAGAUGUAGGUGGCUGUUGUAUAUAUUCAGGGAAGCUUCCUG | |
| | AUAGUAGUGGGAUACAACGUGUGCGGUUUAUCACAUACAUGUACAUGUAU | |
| | AUAACAAGAUUCUUACUAGACUUUUUGGUUAGACUUCCCUACAUUGAAGG | |
| | GGAUUGUAUUCACAAGGUCACAGUUUCUCUUCUCUGCAUAUAGUCAAAGC | |
| | UGCACUCUGAGACAUCUCUGAUUUCUGAUCCCAAGAAUAGCUUGUCCUUU | |
| | GCUUUUUACUUUCCAAUAGGGAAUGGAUGUCCUUUGACUCAGCUAAUCGC | |
| | AGUGGCCCUAUUUCUUCCAACUGUCCUGUUUGUGGUUACAUCAUCUCAAA | |
| | UUGCUGUGAGAUUCAACUCUCCAGUCUCUGUCACAUAUAUAUAAUACAGC | |
| | UCAGCUGAGUGGCAGCAAGCUUUGUUUCGGAACUGGCAACAUUCCAGGAG | |
| | UCUGGUUGUGUGCCUCGGGUAAAUUUUUGUCUUGCUAUUUGUUGUUGGUG | |
| | UGGUUUCUAGGUGACCCUAAUGAAAAUGUCCAGGAAAUUACAUGCGAUAU | |
| | UCCUGACAGUUCCAAGUCUAACAGCUGCUUAUUAGGAUAGACAUCUGACU | |
| | UUAUCUGGUGCCUAAUGAUUUUGUUGUGGUUUAGUAUUCCACCGACUGGA | |
| | UUGCUUAGGUGCUAUUCGUAACAGACACACAACUUAGUCUCUGCCUUCUG | |
| | GGAGCUGAUGAUCCCUAAUUCACAGAGCUUAAGAGGGCACAAGGCCAUCC | |
| | ACAGCAUGUCUAGAAAGCCAGAGAACUUUUCCUUAGCACUCUCAGCUUUU | |
| | GAAUUAAUUUCUCUUUUUUCUUCUUGGUGGCCCACACUCUAAUCUCACUU | |
| | UUUAUUCAUAUCCAAAAGAAUAAUUUACCUCAAACAACAUAAAGAACUAC | |
| | CUCUACUGAAGUCAUGUGAAUUUUAACACUGAAUUAAUCUAAAAAAUAGG | |
| | AAUGUUAUAAUAUUUGAGUUUAAAAUGUUAGUGACAGAGGAAUAAUUUUG | |
| | CACAAUAGAGUCUUCUCUUCAUUACUUACUAUUGGCCCAUGUGACCAUGA | |
| | AUCUAAAUAUGAUCCUUAAUACACAGAUUCACAUUAUGUUAAUGUGUGUA | |
| | UGUCCUUCUCAGUAACCCCUGACAGAACAAGUCAUUUCAUCAAGAGUCGG | |
| | GGAGGUUAUCAGCUGAUCGGUCUGUGAAUUAGUAAUCAUGCUCAGCUGAA | |
| | UGAUUAGACUGUUAUAAAAAGAAGUUAUCACAGUACCUGACUUUGGGUCA | |
| | AGGCAUGCAUUUGAUUAUUGGUAUAUUUUGGGUGUACAUGGCAUAUAGAA | |
| | UUCAGGAAAGAAUAUAUGGGAUUUUAAAUUUAUUUUAAUAUGUAAAAUAC | |
| | CGUAUUGUAUUAGUUAUCUACUGCUAUAUACAAAUUACCCCUAAACUUAG | |
| | UGAAUUAAAAUAACAAACAUUUAUUAAAUCACAAUUUCUGUGAGUCAGGG | |
| | AUUGGAGAGAAGUUAGCUGAGGUUCUGGUGUAGAGUUUCUCAGGCGUCUG | |
| | CAGUCCAGUUGUUGGCUGAGGCUAUAGUUAUCUGAAGGCUUGACAGGGGC | |
| | UAUCAGACCCAGUUCUAAGAUGUUUCACUCACAUGGUUCUUUACAGCAGU | |
| | CCUCAGUUCAUUGUCACAUGGGCCUCUUUAUAGGGUGGUUGAGUGUCCUG | |
| | AAAGCAUGGCAGCUGACUUUUUUCAAAGCAAGGGGUCUAAGAGAAAGAGC | |
| | AAGAACCCUCAAUGACUUUUAUGCCUCAUCAUAUUCCAUCAAUUCUGCCA | |
| | UAAUCUAUUGGUUUGAAGUUUAUUGUUAUAAAUCCAGCCCACAUUUCAGA | |
| | AGAGGGGAAUUCAACUCAACUUCUUGAAGUGAGGAGUAUGAUAUAAAGGA | |
| | GUUUGCAGAUAUAUUUUAAAACCACCACAUGCAUUGAAAGGAUAGAGAAA | |
| | UAAUGCUCAGGAAACUAUCCAACAUAAUUGAGCCACUAAGGAUCCAGAUU | |
| | UGUUUUGAAAGUUUUCUCCUCUGUUUCUCCUAUUACCUGGAAAAUAUCUU | |
| | ACCUUGGCUUCUGAAGCUAUUGAUAUAUAUCUAAUUGCUAUAGGUAUAUU | |
| | CCUAAUAAAGUCUAAACUAAUUACUAGGGAAACGGAACCCACAAAAUUAU | |
| | GUUUAUUUUUCCUUAAUUCUGGAGUAUUAAUGUUAAAUAUGACCCUCCUG | |
| | UCCCAGGAUGUCAGCAGAGGCUUCAAAGCUGGGAACACUAUAUUUUAAGU | |
| | GCUCUCUUGUUCUGAAGACCAGAGACAUUUCCAAAGGACUUGCUAAAGAG | |
| | UGGAAAUAAAAAUGAUCUUAUAAACUUUAGCCAAUGUGACAUGAGAUAAU | |
| | AAAUUUGGAGAAAUAGGAGGAAGAUGCUUUGAAAAGGAUUGUUGGCUUAC | |
| | UCAAGAGGAGACUGUUUUAUUCUAACUCAGCUAAGUGGUGGAGGAUGUCU | |
| | UCUUAUGAUGAAGGGAAACUGCCAUAUUGCCCCUGGUGGUUGUUGGCUGA | |
| | GCUGUGGAAAUGAGCUUGGGUGAUGCUGAUCUGUUUACCCUGGCUUGAAU | |
| | CCCUAUGUGCACAAGGAAAGCUACCUCGAGUAACUGCUAAAGAACAAGUA | |
| | AGGAGAGAAAGCUGCAGUGAGAGCAAAUAACUCCAUUAGUAGGAGGGCAA | |
| | GGGUACACUUACUGGAGCAAGAGAAGGCACUGUGGAAGGAAGUGAAAUAG | |
| | AACCCUUUAGAGAGGAUGUCUAGUGAGGCAAGGUGACCCAGCAAAUGAAG | |
| | GUUCUGUGGGGUGUCAUUAGAAAUGGGGACUGUGAAUAAGCCACUUGAAG | |
| | GCAUCCAGAAAAAAUGUUGCUCACUGGUAAAUGAUGUAAUGUUGAACGCU | |
| | UCUGUGGGAAAGCGUCUCCAGAGAACCCACCAAAACAUCCCUUGUGUGAG | |
| | AGAGCCAAUGUUUGGACAUCUUCCGUGACAGAGGAUUUCCAACAACCAGG | |
| | AGAAAAUACAAAUGAUGCCAUUAAUUUUAAACAGAUUUUUCCAAUUUAAC | |
| | AUUCAUCCCUGUUCCCAACCCACGUCUAGGGGGAGCUAGAGGCAGUGUAG | |
| | UGAGUCAGGAAUGAAGUUGGAAGAGAAAGGUGGAAACAUAGUAAAAUAUC | |
| | AUGAUACGCCCUUGCUUCCAAUUACUGUGAAACUGAGAAUCAGUUCUGAG | |
| | UGAGGCUGGGAAGUGGAAGCUUAAAAUUGAGUGAAAACUGAAUGCUUCGU | |
| | UGUAUAUUAAACUGGAAAGAAGAUGGAAAAGGAGACUAUUCUAAUGCCUG | |
| | AAAGUAAUCAGAAAGCUGUGGAAUCUAAGAUAUGGCCAAGAGGUAAGGGA | |
| | AGGAAAAUACGACAAAACAGGUUUGAGGACAGAUAUGAACAAAAUUAAAA | |
| | AAAAAUCCUACUUAAACUCUUUGGCCCAUUCAAUAAAUUUGUUGCAAAAG | |
| | AGCUUGUUGGGUACAAUGUUUUCGAGUUAACGUCCUGUGGCAUAUUGGAU | |
| | CUCUUCACAGAUGUUCAUCAAGUGAUGGUAAGUGGGAGAAGUUUAUCUCU | |
| | AUUCUCACCCAUCCUAUAAAUGCUGUGGAGUGAAGUGAUGAACUGAGAGA | |
| | CUCAUGUAAAAUAGUACUGAGGAUAAGCUGGCACUGAGCAAGAGGGAAGA | |
| | GGACUAGUAACUAUAUUGUCAAGUACUGGCAAUGAGAGACUGCAUGUACA | |
| | CUGGAGUGUACCAAACAUUGCCUUCCUGGUCAGAGAAAUGAGCAGAUCUG | |
| | AGUAGACAAUUUUUUUUCUUUUUUUUCUUUUUUUUCUUUUCUUUUUUUUU | |
| | UUUUUGAGAUGGAGUCUUGCUCUGUCACCCAGGCUGGAAUCCAGUGGCAU | |
| | GAUCUGGGCUUACCGCAACCUCCGCCUCCUGGGUUCAAGCAAUUCUCCUG | |
| | CCUCAGCCUCCCUAGUAGCUGGGAUUACAGGCGCCUGCCACCACACCUGG | |
| | CUAAUUUUUGUAUUUUUAGUAGAGACAGGGUUUCACUAUUUUGGCUGGGC | |
| | UGGUCUCCAGCUCCUGACCUCGUGAUUCGCCCAUCUCGGCCUCCCAAAGU | |
| | GCUGGGAUUACAGGCCUGAGCCAACAUGCCUGGCCUGAGUAGACAUUUUA | |
| | ACAGGCGGUGACAAGUGAACAGGCAACACUGAAUGCUUUUCUAGACACCA | |
| | UAAGAAAUAAUUGCACUGUGGCUAUUUUUUAUUUAUUUGGGAAUUAUUUG | |
| | GAAUCUUUUAAUCUCUUCAGUUGGACUACUGCAAACUGAUGAUAUUUGAA | |
| | AGUGCACCUGCAACCACUUUUUUUUUUUUGAGAUGGAGUCUUGCUUUGUU | |
| | GCCCAGGCUGGAGUGCAGUGGCGCGAUCUUGACUCACUGUAACCUCCACC | |
| | UCCUGGGUUCAAGGAAUUCUCCUAUCUCAGCCUUCCAGGUAGGUGGGAUU | |
| | AUAGGUAUGUGCCACCAUGCCCGGCUAAUUUUUGUAUUUUUAGUAGAGAC | |
| | AGGGUUUCCCCAUGUUGGCCAGGCUGGUCUCAAACUCCUGGUCUCAGGUG | |
| | AUCUACCCGCCUCGGCCUCCCAAAGUGCUGGGAUUGCAGGCAUGAGCCAC | |
| | UGCGCCUGGUGCGAACCACUUUUGUACAUAACUAAAGGACUGAAGUCAAA | |
| | GGAUGUCCACAGGGAAAAUAUUGAGUGAAAAUAAUAAUAAUAAUAUAUAC | |
| | AACAGUUCAGUAUUGUUCUAAAUGUUUUACAUACAUUAACUCAUUUGUUU | |
| | GGAUACACUUGCCUUGCCCUAUCAUUGGCAAUGAUUUUCACAAAUUUUUA | |
| | AUAUACCUGACAUUAAGCAUUUUUAUAUUUGUAAUCUGCCAUAGACAUCC | |
| | UAUUUAAAUUUAGCUAGUUAAGAUUGCCUUAUUGGUGAGCACCAGGUGCG | |
| | GCGUGGGGCAGGGCAGGGUCGUCAUGGCGGAGUUGCAGCAGCUGCGGGUG | |
| | CAGGAGACGGUGGACUCCAUGGUGAAGAGUCUAGAGAGAGAACAUCCAGA | |
| | AGAUGCAGAGUCUUAUGUUCCCGUGCAGCACCAGCUGUUGUGAGGACAGC | |
| | UAGGCAUCCAUGCAGUAAGUGCACCAGUGCAUCGAGCGCUGCCAUGCACC | |
| | UCUGGCUCAAGUCCAGGCCUUGGUCACCAGUAAGUUGGAGAAGUUCCAUG | |
| | ACCGCCAGGCCUGGUGCACCAAACAUUGCAACGACAAAGCCAAAGAUUCA | |
| | AUAGAUGCUGGGAGUAAGGAGCUUCAGGGGAAGCGGUAGCUGGACUGUCG | |
| | UGUGACCAAGUGUGUGGAUGACCACAUGCACCUCAUCCCAACCAUGACCA | |
| | AGAAGAAGGAGGCUCUCUUACCCAUUGGGAAAUAGAAGUCCUUGCCAGUG | |
| | GCCAUCGGGACUGAGGGCAAGAAUAUAUUUUUUAAUAAGGAAUUGGGAAU | |
| | UUCAGUAUUUUAAGCAAAGUUUAUGAAUGAAGAAAUUAAGGAUGGCAACA | |
| | AGUGUAAGGCAUAUGUCACUUGCCUCCGGACACUGGUUCUUUCAUGUUUC | |
| | AGUCCUAAAAAGUGAAAUAGGAAAAAGUGGUGCUAAAUUGCAUCGGAGAU | |
| | AUUACAGGAAGUUUUAGAGCUUAUGUUCCUGUGGCUAGUGCUUGUCCUGG | |
| | CCGUAAGGAUGUUGCCUGUAAUAAGUCAGAGCCCUCCAAGGUACCAGACU | |
| | CUUCUUACUACACAGGUACCAAUAGGCUGGAAGGUUAGACCUGAUGGAGU | |
| | UUGAGGAGAGAUAUUUUCUCUUUGUUGCCAACAUCCUGUUUACCAGAAGG | |
| | GUCAUCACACCAUUUUCUGUAAGCUGUGAAACAAAAUCCAAGAGGUCAAU | |
| | CACUUAGAAGGGAAAAACAAAUUUCUGGGUCUUAGUUUUCUUGAUUUUGU | |
| | GUAAUUUAUACAAUGGUAUACAAAUUGAUUUUAAGAUGUGGAAUUGGGAG | |
| | GGAGAGUAGUUUGGAUAAGAACUUUGAAGAUCCUUUUGGAUCCCCAUUUC | |
| | UGGUCAUCGAUAUGUAAAUAUACAUUUCUUAAUAUUAUUACAUGCUGUGU | |
| | CUUUCAACCUGGAGACCGUGCAAAAACAUAAGAGGUGACGACACACUAAU | |
| | UGUGGGAAGCAGAGCUUCCCUCUUACUGGCUGAUGGGCCCUGAGGUUGUG | |
| | UGUAGCAAAAUGACAGGACGAAUCUUGCAGUAACACUUUCCCCUUGAAGA | |
| | GAAGGGGGUUUUGACUGUGAUAUAUAUUAGUAUCUAGGAAUGAAUAGUAA | |
| | AAGAGGAGCAAUUGGCUACUUGCUUAUAAGGGAGUUAUGAAGUACUGGAU | |
| | UUGGAAAAACCUGGUUUUUAUAAAACAGAAUGGAAUGAAAGCCUAAACCU | |
| | AGCGUUGUUUCCUUAGCUCCCUGAAUUAACAGAGCCCAAUUGAGAUAAAC | |
| | CCCUGGCAACAGGAAAUUCAAGGGAAAAAAAGUAAACAACUCGGGCUAGG | |
| | AUGAGCUGACUCCCUUAGAGCAAAGGAGAGGCAGCCAGUCCCCAUUAUCA | |
| | AAUACCAUUUUUGCCCGGGGCUUUUGAAACUUGCAGUGUUUCAAAACUGG | |
| | CAAGGCACCUUAUUAUUUUGAUACCAACCUCAUUGUAUUUUCACCAACUU | |
| | AUUACUUGAAAUGAUAAUACAGCCUGUCCAUUUGCUGUUUUCAGGCUGUG | |
| | AUAUAUUUUCUUAGUAAUUUGGUUUUAAAAAUAAAUAAGGUUUAAUUCCC | |
| | CCCUCCCCGCCACAAAAAAGAUUGCCUUAUUCAUUACUAAAGAAUGAUAU | |
| | AUCACUUUUUAGAAGUAUGUGAUGUUUUCUAACUACAAUUUUUACAAACU | |
| | AGGCUUUUAGAAUUGCCAUUUAUGAUCAUUCUUGCAAUGCAAACUAAAAC | |
| | UAAAAUACGUUGCUGUUUAGGUUUAUAAAACAAGUGCCAUUCAUCAGGGA | |
| | AAAGAUAGAAUACGUCUUUUCUUUUUCUUUCUUCUUAGAAGAGUUUUGUA | |
| | UAAAGAACUUCUUUUAAAUAUUUGAGAUACUUUUCACCCAGAAGAGGUGU | |
| | UGUUUAAAUUGGGGUUUGUGACUAAUUUGUUUCAAUUAAAAAUACUUUAA | |
| | UAUCAUUAUCAAAUGCCUCUUAGAUGAUAUGAUCUCUGUUACCAUAUGUG | |
| | GUAGUUUGCUCUACAUACUAGCUAUUUAAAACAGAUGUAAGUAAAACAAG | |
| | AUUUGUUUGGGAGGAAUAGGGUUUCUCCAGAGUAAAGAAUACAGAGACCA | |
| | AAUGGAAACAUACUUGCUUCACUGCACAUGGUUUCUAUACUUGGCUGUGC | |
| | UGGCUUACCAUUUUAUGGUUACCAGAUUUACAUCCUUUGAAAUUAUGGAC | |
| | AUUGGAUCAAAUCCUCAUGUUGGAAAACACUCUAAGGUAACAUUAAAUGA | |
| | GCAUGUGAUUUGAAUAACCUACAAUUUUCACACAGGUUUUGAUGACCAAA | |
| | GUCAAAAUAUUUCCCAUAAAAUACUGUAAAUUUGCUAAAAUUUAUACAAA | |
| | GCUCCUACAGUGUAAUAAUACAAAGAAACUUUGUUUACUGAAAAAAUCUA | |
| | AAAUAUCUUUUUUUAUAUUUUAUUUUAAACAAUUUUUUAAGUUUGUAUUU | |
| | UUAUCUUAUUUAUUUAAGCUUGUUUUCUCUCUCUUUUUUAAAAUAAUUCU | |
| | AAAAAAACUAGAUACAAAUGCAGAGCGUGCAGGCUUGUUACAUAGGUAUA | |
| | CGUGUGCCAUGGUGGUUUGCUGCACCUAUUGACCCAUCCUCUAAGUUCCC | |
| | UCCUCUCCUCCUGUCACCUCCCACCCCCUAAAGGGCCUUGGUGUGUGUUG | |
| | UUCCCCUCCCUAUGUCCAUGUUUUCUCAUUGUUCAACUCCCACUUAUGAG | |
| | UAAGAACAUGGGGUAUUUGGUUUUCUGUUCCUCUGUUAGUUUGCUGAGGA | |
| | UGAUGGCUUCCAGCUUCAUCCAUCUCCCUGCAAAGGACAUGAUCUCAUUC | |
| | CUUUUUAUGACUGCAUAGUAUUCCAUGGUGUAUAUGUACCACAUUUUCUU | |
| | UAUCCUGUCUAUCAUUGAUGGGCAUUUGGGUUGGUUCCAUGUCUUUGCUA | |
| | UUGUAAAUAGUGCUGCAAUAAACAUACGUUUGCAUGUGUCUUUAUAGUAG | |
| | AAUGAUAUAUAUCUUCCUUUGAGUAUAUACCCAGUAAUGGGAUUGCUGGG | |
| | UCAAAUGGUAUUUCUGGUUCUAGACCUUGAGGAGUCUCCAUACUGUCUUC | |
| | CCCAAUGGUUGAACUCAUUUACAUUCCCACCAACAGAGUAAAAGCAUUCC | |
| | UAUUUCUCCACAGUCUCGCCAGCAUCUAUUGUUUCCUCACUUAAUAAUCA | |
| | CCAUUCUGACAGGCGUGAGAUGGUGUCUCAUUGUGGUUUUGAUUUGCAUU | |
| | UCUCUGAUGAUCAGUGAUGUUGAGCUUUUUUUCAUAUGUUUGUUGGAGAC | |
| | AUAAAUGUCUUCUUUUGAGAAGUGUCUGUUGAUAUCCUUUACCCACUUUU | |
| | UGCUGGGGUUGUUUUUUUUCUUGUAAAUUUGUUUAAGUUCCUUGUAAAUU | |
| | CUGGAUAUUAGACUUUUGUGAGAUGGGUAGAUUGCAAAAAUUUCUCCCAU | |
| | UCUGUAGGUUGCCUGUUUGUUCUGAUGAUAGUUUCUUUUGCUGUGCAGAA | |
| | GCUCUUUAGUUUAAUUAGAUUCCAUUUGUCAAUUUUGGCUUUUGUUGCAA | |
| | UUGCUUUUGGUGUUUUUGUCAUGAAGUCUUUGCCCAUGCCUAUGUCCUGA | |
| | AUGGUAUGGCCUAGGUUUUCUUCUAGAGUUUUUAUGGUUUGGGGUUUGAC | |
| | AUUUAAGCCUUUAAUCCAUCUUGAGUUGAUUUUUGUGUAAGGUGUAAGGA | |
| | AGGGGUCCAGUUUCAGUUUUCUGCAUAUGGCUAGCCAGUUUUCUCAGCAC | |
| | CAUUUACUAAACAGGAGAUCCUUUCCCCUGUUUAAUAAAUUUUAUAUAAG | |
| | UUUAUUAAAAUUAUAGAAUUGUAGAGCUAGAAGAUUCUUAGAAGGUCUAU | |
| | AGUCACUAUGUUAGCAGAUUCUUAGAUUAGUCAUAUUUGACUCAGCAAGU | |
| | ACAGUGUUGUAUUUGUAUUAGGUUCUUUUUGUGUUAGUUUCUUCUUUGUA | |
| | UUUGUAUUCUUGAAUUUGUAUUAGUUUUCUUUUCUUUCUUUUUUUUGGAG | |
| | ACAGGGUCUUGCUCAGUUGCUCAGGCUGCAGUGCAGUGGCAUGAUCACAG | |
| | CUCUCUGUAGCUUUGACCUCCUGGGCUCAAGCAAUCCUCCCAGCUCAGUC | |
| | UCCUAAUUAGCUGGUACUAUAGGUGCACACCAUCAUGCUUGGGUGAUUUU | |
| | UUUUUAAUAUUUUUUGUAGAGACAGCGUUUCACAUGUUGCCCAGGCUGGU | |
| | CUCAAAACUUGGGCUCAAGUGGUCCGCCUUUCUGGGUCUCCUAGAGUGCU | |
| | GGGAUUACAGGUAUGAGCCACCACGCCCAGCUUGUAUUAGUUUUCUCUUA | |
| | CUGUGCCACAAAUUACCACAGACUGUACAGCUUAAAACAACAUGUAUUUA | |
| | UUAUCUUACAGUUGUGUAGGCCAGAAGUCUAGGUACAAUGUCACUGUAUU | |
| | CUCUUCUCAGGGUUUCUUUAGACUGAAAUCAAAGUGUUGUCCAGGGCUGG | |
| | GCUUCUUAUUUGGGAUUUGGGAUUCUCUUCCAAGUUCAUUGUUUGUAGGC | |
| | AGAAUUCAUUUCCUUGAAAGCUUAUUGACUGAGCUCCCCAUUGUCUGCUA | |
| | GCUGUCAAAUCUCUGACUUCCUCUCCUGAAACCAGCCAGAGAAAACUCUU | |
| | GGUUUUUAAAGGGAUCAUGUGAUCAGGUUAGGCCCACCUCGAUAAUACAG | |
| | UUUUUGCCCUGUAAUAUAGUUUUGGAGUGAUAUCUCAUCAUAUUUACAAG | |
| | UUCUGCUCACAUUCAAGGAGAGGAGAUUUUACAAGGGCGUGGGUCAUGGG | |
| | GAACGAGUUACCUUUUGCCUGACACACACACAUACACACACACACACACA | |
| | CAUAUAUAUGUAUGUGUGUAUAUAUAUAUAUAUAUAUAUAUAUAUAUAUA | |
| | UAUAUAUAUAUAGCAAUGACUGUAACUUUAUGGUCUGCGGGAUUAUUUGU | |
| | AUCUAGGAAAUCUGCCUUCUGAACAGUGGUCAGAAGGUCUGGCUUGUCUG | |
| | GUUAUUGCCAUCUAUUCCUACUUCAUUUUACUCACAUACCCCUGGCCCUA | |
| | AUGUGCUUUUUGGGAAGUAGGCAAUACUCUCUCGUGGGUAUCUUUGGCUU | |
| | AGUGGUGCCAAUAUUCCAGGCUUAAACUUCUCCUCUCUCCUUUGUUCUCA | |
| | CACUGGAGACUACUUCAAUGUUUAUGGCAAAUGUACACACUCUGGUUUCA | |
| | UAGUAUUUAAAAUAAAGUGACCAUAUUUCCUACUUAUAAGACAAUUUUUG | |
| | GCUCAGAAAUGUAUACAGAAAUACACUUUUUAAAUAAUUUUCAGGCACAG | |
| | UUUCAAAUACUCACUAAAUUGCUCCUUUCCCAUUUUUCCUAUAAUGCACA | |
| | AUAAUGUAUGUCCCUUGAUCUAGUCCCAGAAAAGGUUUCCAGGAAGAUUU | |
| | CUGAGAAAAGCGCAUAUGUGACAUUUUCUGAAAUGAACUUGCUCAGCAAC | |
| | AUGUACUUUUUUCCUUUUUGUUAAUGUCCCCAUAUUUGAGCAGAAGGUAA | |
| | AAUUUUGUUUACAUGUAUAACUUGCUUCAAUUUUUCACACACGUUUUCUA | |
| | AGAAUGUGCAUACUGAAGUUGGUGGGUUAUAAAAGGGAUUAAUACCCCUU | |
| | UUGGCCUAUAUUUAUUGGAAAGACCAAGUUGCCAGGGUUCUUUGAAGAAU | |
| | UCAGACAAAUACAUCUGAAUAUUUUCCAUCAAACGGGAGGUAGAGGAUCA | |
| | AGCAUGGAUUUGCUCUUUAUAGCACUUCUGGCAAGAGGCUUUUGGAAUAA | |
| | AAUUUAUGUAUUCCUUCUCAAUUUGGUUAACCGCAACAUCUGUUUGUCUC | |
| | CAUUGGCCUUUGGCCAUUGACCUACUGUCUCCUGUAGAAGACACCCACCA | |
| | UCACUCUUACGACCAAAUGUUGAUGGGAAACAAGCAGUUCUCAAGUGAUU | |
| | UUAGUCAUGGAAAUAGUGAUAAGCCAAAAGCAUGGCCUAAUUAUAUCAUC | |
| | UAAAUUCAUGGAAAAGGGAAUUAAUUUUAUAUUUUAUCUGAUUUUGGGGA | |
| | CUGAAUUAUUAAUAGUACCAACUUAUUUGGACAGGAAAAGGCCAAGAGAU | |
| | UUAGACAAGAACACAGUAAACAGACCCUUCUCAUUCAAUAUAGCAUGGGA | |
| | CAUGUAGAUUUACUUUUUGACUGCAGGAACAAUUAGAGGAAUCUAGGUGA | |
| | UUUUUCCAAACUUAGGAAAUGAAAGUUUAACUAAAAUUAAAUAUGUCUGU | |
| | AUGUUUAUACAUUUUUGAGAUAAAACACUAUUUUCUCUAGUUUUUUAAAA | |
| | AUGAUUUUUUCUAAUUUCUAACAAUAGCUUAUAAGUUUACUACACAAUUA | |
| | UUGCUACUUCGAAAACGGAUUUACCACUGUUAGGCAGAACAAACAAAGGU | |
| | UCAGGAGAGGCCAAUGCCUGUGAGACCGUGGUACCAGGAUCUUCCAGUAA | |
| | CAACUUGGAGCCUGCCAGGCUAUCCUUUGAUCAAGGGCUCUCUCAGUGUU | |
| | GGUACCUCUUGCUGCCUGGUCCUGCUUUGUCCUGGCCAGCCAUACCCUGC | |
| | CCAGCUGUGAAAGACCAUUCAGUUUCUGCUUUUUCUUUAAUUUUUAGAUU | |
| | UUUUUUGUUUACCUCAAAUUAUUCCAGUUGCAUGAUAGCAAAAGUAAGCC | |
| | AGGUCCCUUUAAGAAGCACUGGACUGAAAAUCCAGCUUCUGCUAUAAAAA | |
| | CUCAUUUCCCCACUCAAGGCAUUUUCCUUCUGAUUUCAGAGGUGAGGUAC | |
| | UCAGAUGUGCUUUUGCCUCCAAUCUCAGCAUCUUCUCCUAUUCAUUUGUU | |
| | GCCCAUUCUAUGCUUUCAAUUUCUGCUAUAGCCACUCAGUCUUCCUCUGU | |
| | UCUGGUCUCAAGGUGAGCAUUUUCUGCUACCCCUGUGUCUAGUGACACCU | |
| | CCCAGUUUCUGGCACAUAACUCUUUUCUCUGGUUCUCUCUUAAGACCUUG | |
| | GUUUGUCUCUGGCCCAAUUCAGGCCACGAUUUCUACCCACCUUGACCUUU | |
| | GCCAUGGUUGGAGCUGUAUCUUAUGACACUGUGUUAGCUGUAUCUUAUCA | |
| | CACAAACCUGUGGGAACAAGAGAUGUAGUAUCUACCCACAAGGAAUCUAC | |
| | AGACUACUGGGAAUAGCAGACCCUCAUUAAUUAAUUAUUAGCAUUAAUUC | |
| | UUCAUGCUUGUAAUCCUAAAUUUUUUAAAAAUUUGAGAACAAUUUUUUUU | |
| | UCUGUCUAGUUUGGGUGUUGGCUAUUCCUAAAUAUUAUGGUACUUACUUG | |
| | UUUACCUGAAGGCUGGUUUCUCACCUGGCUGCACCUCUGCUGGGGGGUGC | |
| | UCACUUUGUCCCUAACUUUUGGGUCAAUAGGAAGAAGUAAGACAUUGAGU | |
| | GAAUAAAUUUGACCUUAAGCUGCUCUUUGCCAUGGAGGAGUCCUGGCCCC | |
| | UAGUGUUUGACUGUCAGUGUUUCUUUACCCCAUAAAAAUUUUCAUCCCUA | |
| | AUAAAAUGCUGCCAUGUAACUGACUCUACAUGCUGAACCAAGAGAAAUUA | |
| | AGAAGAAUAAACCUAAUUUGCUUCAGAAAAGAAUGUUUCAAUAUAGAGGA | |
| | AUUAUUUCUUAUUUUAACCUUGAUAAUUUUGCCUAUAACAUUUUCUGGAA | |
| | AAUAGAUAAGUAUGCAGAGUCUUAGUUAUAUUUAUAGUAAAAUAAAAUAU | |
| | AGCAAGGUUAAUGGAAUUGUCCAGAGACCCACAACGAACAGAGGGCAGAU | |
| | CACAGAACUCCACACUCAUACUCAUGUUCUAGCCAUUAGGCCUCUUGGCC | |
| | UCUCUUUGUUCUAUUUGCCAAGGUAUGACCAGAGCAGAAAACAUCCUCAA | |
| | CCAAUUAGGCAAGCUCCCCAUCUGGUGAUUAGUCUUGCUAUGAAGACAUC | |
| | UUGUGUAUUACCACAUUUACGACUGCAAUUGGAGCUUUAUUUACCAUUGG | |
| | GGGUAUAGCUCUUUCCGAGGAAAUGACCUUCUAACUGAGCCAGGAGGGGC | |
| | UGGGCUAAGUUUGUUUCAGCUGGAAAUUACACAUUACAUAUUUAGUAUGU | |
| | UUGGUCACUUUAGAUCUCUGUGAGGCAGGGAAGGAGAAUUUUAUGCAUUA | |
| | CAAAGUUCCUAAAAAUAUUUACAAUCUUGUAAAUCAAAGUGGGUUUCUAC | |
| | UGGAUUUAGCUUUAUGCAAAUCUUUAUAUUAUGUCUGGGCUUUCAGUCUG | |
| | AUUUUAGAAGAGUACUAUUACAGUAUUUUAACUUUUAUUGAAAAUCCAGG | |
| | CUUUUUAAUUUUUUUAAUUUUUAAUUUUUGUGGGUACGUAAUAGGUGUAU | |
| | ACAUUUAUGGGGUACAUGAAAUAUUCUGGUAUAGGCAUGCAAAGCAUAAU | |
| | AGUCAUAACAUGUAGAAUUGGGUAUCCAUCCCCUGAAGCACUUGUCCUUU | |
| | CUGUUACAAACAAUUAAAUUGUAUCUUGAGUUAUUUUAAAAUGUACAAUU | |
| | AAAUUAUUUUGACGACAAUCACCCUGUUGUACUAUGAAAUACUAGGUCAU | |
| | AUUUAUUAUUUCUAUCUUUUUUGUACCUAUUAACGUCUCAAUCUCCUUCU | |
| | CACUACCCUUCCCAGUCUCUGGUAACCAUUCUUUCAUUCUUUCUCUCUAU | |
| | GAGUUUAACUGAUUUGAUUUUUAGAUGCUACCAGUAAGUGAGAACGUGAG | |
| | AUGUUUGCUUUUCUCUGCCUGACUUAUUUGACUUAAUGUAAUGAAUUCCA | |
| | GUUCCAUCCAUGUUAUUGCGAAUGACAGGAUUGGAUUUAAUUUUCUUUUA | |
| | UGGCUGAAUAGUACUCCAUUGUAUAUAAGUGUCACAUUUUCUUUAUUCAU | |
| | UCAUCUGUUGAUGGACACUUAGGUUGCUUCCAAAUCUUGACUAUUGUGAA | |
| | CAGCGCUGGGACAAACAUGGGAGUACAGAUAUAUCUCUUCAAUAUGCUAG | |
| | UUUCCUUUUUUUGGGUAAAAACUCAGCAGAAGGAUUCCUGGAUCGUAAGG | |
| | UACCUCUGUUUUUAAUUGUUUUGAGAAAACUCCAAACUGUUUUUCAUAAU | |
| | GGCUGUACUAAUUUACCUUCCCACCAACAGUGUAUAAGGGUUCCCUUUUC | |
| | UCCAUAUCCUCGCCAGCAUUUGAUAUAGCCUGACUUUUCUUUUUUUGAGA | |
| | UAGGGUAUCACUCUGUCACCCAGGCUGGAGUGCAGUGGUGUGAUCUCAGC | |
| | UCACUUCAGCCUUGACCCUGUGGGCUGAAGUGACCUCCCACCUCAGCCAC | |
| | CCGAGUAGGUGGGACUACAGGCAUGUGCCACCAUGCCUGGCUUGCCUGAC | |
| | UUUUAGAUAAAAGUCAUUUUAACUGGAGUGAGAUGAUAUCUCAUUGUAGU | |
| | UUUGAUUUGCAUAUCUCUGAUGAUCAGUAAUGUUGAGCACUUUUUCUUAU | |
| | GCCUGUUUGCCAUUCAUAUGUCUAUUUUUGAGAAAUGUCUAUUUAAAUCU | |
| | UUUGCCAAUUUUUAAAUCAGAUUAUUAGAUUUUUAAGUUUUAUGAAGUCU | |
| | AGAAAUUUUAGAGUUGUUUGAGCUUCUUAUGUAUUCUAAUUAUUAAUCCC | |
| | UUGUCAGAUGAGUAGUUAGCAAAUAUUUUCUCACAGCCUCUGUGUUAUCU | |
| | GUUUACUUUGUUGAUUGUUUGCCGUGCAGAAGCUUUUUAAUUUGAUGUGA | |
| | UCCCAUUUGUCUGUUUUCGCUUUGUUUGCCUAUGCUUGUGGGGUAUUACU | |
| | CAAGGAUUUUUUUUUGCCCAGACCAAUAUCUUGGAGAGUUUCCCCAAUGU | |
| | UUUCUUGUAGUAGUUUCAUAGUAGUUUGAGGUGUUAGACUUCAGUGUUUA | |
| | AUCCAUUUUGCUUUGAUUUUUGUAUAUGGUGAGAGAAAGGGAUCCAGUUU | |
| | CAUUCUUCUGCAAGUAGAUAUCCAUGUGGAUAUUCAGUUUCCCCCAUACC | |
| | GUUUAUUGAAGAGACUGUCUUUUCUCCAGUGUAUAUUCUUGGCACCUGUG | |
| | UCAAAAACGAGUUCACAGUAGGUGUGUGGACUUGUUUCUGGGUUCUUUAU | |
| | UCUGUUCCAUUGGUCUAUGUGUCUGUUUUUAUGUGAGUACCAUGCUGUUU | |
| | UGGUUACUGUAGUCCUUUAGUAUAAUUUGAAGUCAGGUAAUGUCAUUCCU | |
| | CCAGUUUUGUUCUUUUUGCUCAGGGUAGCUUUGGCUACUCAGGGUCUUUU | |
| | GUGGUUUCAUAUAAAUUUUAGAAUUGCUUUUUCCAUUUCUGUGAAGAAUG | |
| | UCAUUUGUAUUUUUAUAAAAAUUGCAUCAAAUCUGUGGAUUGCUUUGGGU | |
| | AGUAUGGACAUUUUAACAAUAUUGAUUCUUCCAGUCCAUGAACAUAGAAU | |
| | AUCUUUCCAAUUUUUUGUGUCUUGUUCAAUUUCUCUCAUUAUCAUUUUAU | |
| | AGUUGUCUUUAUGGAGAUCUUUCACUUUAAUGGUUAAUUCUUAGUUAUUU | |
| | CAUUUUAUUUGUGGCUAUUGUAAAGGGAGUACUUUCAUUAUUUCUUUUUC | |
| | AGAUUGUUCACAGUUUUUAUAUAGAAAUGCUACUGAUUUUCAUAUGUUGA | |
| | UUUUGUAUCUUGCAACUUGACUGAAUUUAUCAGUUUGAAUAGUUUUUUUG | |
| | GUGUUGUCUUUAGUUUUCUCCAAAUGUAAGAUCAUAUUAUCUGCAAAGAA | |
| | ACAUAAUUUGCAUUCUUCAUUUCCAAUGUGGAUGCCCCUUAUUUUUUUUU | |
| | CUUGUCUGAUUGCUCUAGCUAAGACUUUCCUUACUAUGCUGAACAACAGU | |
| | AGUGAAAAUGGGCAUUCUUGUAAUGUUCCAGAUCUAAAAGGAAAGGCUUU | |
| | GAUUUUGUCCCUGUUCAAUGUAAUACUAGCUGUGGGCCUGUCAUAUACGG | |
| | CUUUUUUAAUGUUGAGUUAUGUUUCUUCUAAACCAAGUUUUUUGAGGUUU | |
| | UCUAUUAUGAAGAGAUGUUAAAGUUUAUCAAAUGCUUUUUCGACAUCAAU | |
| | CGAAAUGAUCAUAUGUUUUUUGUCCUUUAUUCUAUUAAUAUGUAGUAUCA | |
| | CCUUGAUUAAUUUGCAUAUGUUGAACCACUCUUGCAGCCCUGGGAUAAAU | |
| | CCCACCUGGUCAUGAUGAAUGAUCUUUUUAAUGUAUUGUUGAAUGUGGUU | |
| | UGCUAAUAUUUUGUUGAGGAGAAAACCUAGACUAUUUCAAUGUGUUUAUG | |
| | CUUAGCUAUCAUGCCCAAAUCAAUUUAGUACAUCGUUUAAAGAUUCCAGA | |
| | UACUCUAAAUCUGGCAACCAGGAAAGAAAACAGCCAUAAAGCAAUGUAUA | |
| | UUUAGAUACCAAUAUUUCCCUCACACUUUCUUUUCGCUUCCUUGUUUCAU | |
| | UUCUUUACUAAUUUGUUGAUUAUUGGCUUAUGACAUAUUGAGGCCUCAGU | |
| | CUAUAAUAACCAUUUUUAUCUGAUGUACAGCUCUACACAUUUUAUUUAAC | |
| | UCUGUGAUGGCAAACUCCUGGUACUUUUGUCAUUCUCUUGCCUCUUAGGC | |
| | UUAUGGCAGAUAUCACUAAACAGUGACAAUAUUCUUUGCUACUAACCUGU | |
| | GGGUUUGGCAUACACAGUAGUGUAUGCAUUAUCAACCUAUAGAAGUGGCU | |
| | UAUAAGUUUAAGUCUGCUAGCUUUUUCUACUCUUUUAGCAUUCUUAAACG | |
| | CUGUUUAAUUAGAUGUAAAAGAAGGAAAUGAGAAGUUAAAUUUGGACUAA | |
| | AUGUGAUAAAAGAAAAUCUUUAUUAACAUAGGAUAUCAGUGAGUCUGAUG | |
| | AAAAGGCUUUAAAAUGCUCUAGGAAGUAUUUCUGCCUGGGAUGUUUGAAU | |
| | UAAACAGUCUCCAAAAGUAGAUCAUUCUCCUUUUGUUUGAAUAUUUUCAG | |
| | AAAUGAUAAAUACACUUUUUGCUGUUGUUGUUGUUUUGAGACACCCAGGG | |
| | UGGAGUGCAAUGGCAUGAUCUUGACUCACUGCAACCUCCACCUCCCAGGU | |
| | UCAAGCGAUUCUCCUGCCUCAGCCUCCUGAUUAGCUGGGAUUACAGGCAC | |
| | CCACCACCACACCCGGGUAAUUUUUGUAUUUUUAGUACAGAUGGGAUUUC | |
| | ACCAUGUUGGUCUCGAACUCCUGACCUCAGGUGAUCCACCUGCCUCUAUC | |
| | UCUGAAAGUGCUGGGUUUACAGGCAUGAGCCACUGUGUCUGGCCUAGAAA | |
| | UAAUAAAUAUACUGUUUAAUGAAAUAGCUUAUUCUAUGUUUGAAGUUUCU | |
| | UAAGUUUAGAAAAUUUUAUGUCGAACUGAAAUUUCUUUUUCUAUAACUUA | |
| | UACCAAUUUUUCUUGUAUUCUUUCAAUGGAAAACAAUGUUUUUUAUGUUU | |
| | UAUGUUUUUAAAAUUAAGAUGUCAUUUAUUUAAAAUAGAAUAAAAUAUAG | |
| | AGGUCCUAAGAUUUCAGUUUGAUGACUGUUGAAAAUUGUGUACACCUAUG | |
| | UGACUACUACCCAACAUAAGAUAUAGGACAUUAGUUUCAUUCCAGAAAGA | |
| | UCGCUAUUCCUGUUUCAAGUUAAUUUCCUGCUCUUCGUCCCAUAAAGGCA | |
| | AUCAGUUUCUUACUUCUGUCACCAUUGAUUAGUUUUAGUGAUAAAAAUGG | |
| | ACUUAUACAGUACGUAUUAUGUCAUAUGUAUAAAUAGAUGUAUUAUGACA | |
| | GUAAGGUUGUUAUGAACAUUUCUGUAAAAACUUUAAUGAACAUAUGUUUU | |
| | CAUUGCUUUUGGGUAAGUAUAUAGGAAUAAAAUUGCUAGGGCAGAAGUUA | |
| | UGUUUAACUUCUUUAGAAACAGCCAAACAGUUUUCCAGAUUGAUAGUACC | |
| | CUUUUGCAUUCUCACUAGCAAUGAAUGAGUUCCAGUUGCAACAUGUCUUG | |
| | CUAGUCAUUUAAGUAUAGCCAUUCUAGUAGUUUUGAAUCUGUAUUUCAUU | |
| | CUGGUUUCAAUUUGUGUUUUCCUGAUGACUAUACAUUUUAGCAGAUUGAU | |
| | GGAGUUAUUAACCCUUUAAAAUAAAUGUAAACUUCCCAACCCAUUUUUAU | |
| | CUAAGCUAGUACUAAUUUAAAUCUCCCCUUACGUGUGCUAUUGUUUGAAA | |
| | UAAAAUGUAUGACAUUAAAUUUACUUUAAUAACAUUAGAUAUUAUGUAUG | |
| | AUUUUCACCUAUUAAGAUAAUUUUAAUUUUGUCAUUGUAUUAGUUACAAA | |
| | UAUGUAUUAUAGAUCUGUGGCUAAUUUAACCAAAAAUACCUGAAAUACCU | |
| | GGCUUUAUGUCUGUACCACUACUUCAUAUUGAGGGACAAAGCUAUGAGUA | |
| | GAAUCCUGCACUUCCUCAUUAGAAGAAGCAUUUUUAAUUAAUAUUGACUC | |
| | UUGAGAUGAUUGUUCAACCUGCUUUUAAAACUUCUGAAUUUUUUCAUCAA | |
| | CCCAUAUUACUAUAUCUCAUUGAUUAAAUGCUCAUGAGGGAUUUGACCAA | |
| | UGACUUAGCAGUGUCAAAAUCUGCUAAACUAUGAGAAUAUGCUGUUCUAA | |
| | UAUCCUAAAAAUGCUGUAAAGGUGGAGAUUAUUUCAGAUAAUAGCUCCUA | |
| | AUUAUUGCAAAACCAUGAUAUAACAAAGAGAUCACUAGACUUGUAAUCGU | |
| | AAAUAUUUCAUUUUCUAGCUGGAUUACCAGGAAAAGUCACUAUAUUUAUC | |
| | UGAAUUCUAUAUUUCACAUCUGUUAAAUGGAGAUAGUAGUAUCUAUUUUG | |
| | UCUACCUCAUAGGGUUGUUGUGAUUAUCACACUAGAUAUUGUGAUUAUCA | |
| | CACUAGAUAUUGUGUGUAAAGUGUUUUGUAACUGUAAAAAAAUACAUAUG | |
| | UAAGAUAUUAAAAGCUUGUAUGUUGAUGUCGCCAGUUUAUAGCAUAGUGU | |
| | UUGCACAUAGUAGGUGCUCUAACAAAAGUAGCUUUUGUUAAAUGAUGGCU | |
| | GGAUGAACAAUAUCUCAAAGCAAUUUUGUUAAUAAACAAUUAUAGAAAUU | |
| | GUUAAAUAUUAUAUCAAUAUUAAUACAAAAAUUCCUGCUCCACUUUAGUA | |
| | AAAGUCACAUUUGUUCAUUUAUAGUCUUUUGGAAUCCCUCAUAUUCUUUG | |
| | UGACUACUUGUGAUAAAUGUCAACAAGUUUGACAUCACUCAAGCAGGUAC | |
| | UUGAAGGGGCCCUAGCAGCAAGUCAUUUAUGGCAGAAUUCCAUUUUUAUA | |
| | AAUAUCAUGGGCUUGCCUUCCUACUCCUUAUUAUUGAACUCACCUUUAAU | |
| | UUGAAGACUAUUUUUAAAAUAAGAAAUAUUUAAGCAAAAAUUUCUAUUUU | |
| | UCUUGAUCUUGAGUACCUGCUCAUUGCUAUUUGUUACCUUUCCCAAUAUC | |
| | AUUAGGUGGUAGUUUAAUUUCUUUGUUUGUUACUGUUCUGAGCAUUUUUA | |
| | AAGUGCUUGACUGAUUUAUUUUAAUCCUGCUAUUAUAUGUUUUCCCCAAG | |
| | UCUCAGGCCUUAUCUUAUUUCCUCUCGCAGUAGCAUUUGACACAGUGGAC | |
| | CAUGCCCUUUUCCUUGAAACUCUUCAUCAUCACUGUGUGGGCUCCCCAUU | |
| | GCCUGAUUCUUCUCCCACUUCACUGUCUGUUCCCUCUAAGUGUCCUUAAC | |
| | UGCUUCUUUUUCAUCUCCCUCCAUGAGACUAUCAGAAAAUUUAUUACCAG | |
| | AAAUCUUGCAGAACAGGAGUGAGUGGGAUGAUAUAUUAAAAGCAGUAAAA | |
| | GAAAAAAAAUCUAUCACAUGAUGAUAAAAUGGUCAGUUCAACAGCAAGUU | |
| | AUAACAAUUACAAAUAUACACAUACCCAACAUCAGAGCACCUAAAUAUAU | |
| | AAAGCAAAUACUAAAAGUUACAAAGGGAGACUGACAAAAAUACAAUAAUA | |
| | GUAGGAUACUUCAGUGCUGUACUUUUAAUAAUAGAUAGAACAUCCAGAUA | |
| | CAAAAUCAAUAUAGAAACAGCUAACUUGAACAAUACUAUGUACCAAGUGG | |
| | ACCUAACAGACAUAUACAGAUCUUUCCAUCCACCAGUAGAAGAAUAUAUA | |
| | UGUUUUUCAAGUGCACGUGGAAUGUUCUCUAGGAUAGAUCACAUUACAUC | |
| | ACAAAACAGGUCUGAUCAAUUUAAAAAGAUUAAAAUCACACCAGUUAUAC | |
| | UUUUCAGCCACAAUAGAAUAAAACUAAAAACAAUAACAGUAAAAAAAAUG | |
| | AGAAAAUUCACAAAUACAUGGAAACUAAACAACACACUCUUGAACAAACA | |
| | UUGGGCAAAAGAGAAAAUAUAGUGGGAAUGUAAAAAGUAUCUCAAGAUAA | |
| | AUGAGAAUACAACCUACGAAAACUUAUGGAUGCACAAAGGCAGUACUAAG | |
| | AAAAAAGUUUAUAAUGGCAAACACCUAAUUUUAAAAAGUAGAAAAUUUAA | |
| | AAUUUAAAUUUAAAAAUAUUUUAAAUUAAAAAAUUUAUCAGAAAAAAUAA | |
| | UAAGAUGACAGUAGAAAUAUGUAAAAUUCAGAAUAGAAAAAAUUUGAAAA | |
| | ACUAAGAGUUUUUUUUGAAAAGAUAAACAUUGAUAAACCCCUAGCUAUGC | |
| | UAAUAUAAAAGAAGAUUCAAAUAGAAUUAAAAAAGAAGAAAUUAUGAGAU | |
| | GACUUAUAAAUAAAAGAAUUAUAAGAUGCUGUUAUGAAAAAUUAUACUCA | |
| | AAUAAAUUAAAUAACCUUGAAGAAAUGGAUAAAUCUUUAAAAACAUACAA | |
| | CCUACCAAAACUGAAUCGAGAAUAAAUGGGAAACCUAAACAGAUCAAUAA | |
| | UAAAUAAGGCGAUUGAAUCAGUAGUCAGAAACCUUCCAAGAAAUAAAAGC | |
| | CCAGGAUCAGAUAGCUUCACUGGUGAAUUCCACCAAAUAUUCAAAGAAUA | |
| | AUUAAUGCUCAUUCAUUUCAAACUCUUCCAAAAAAGUGGAAGGGGGGAAU | |
| | ACUUCCAAACAUAUUUUUAUGAGGCUGGCGUUAUUCUGAUACAAAAGCCA | |
| | GAAAAAGAGAACACAAGCAAAGAAAACAUAGGCCAGUAUUCCUGGACAUA | |
| | AAAGCAAAAAUUCUCCACAAAAUACUAGCAAACUGAAUCCAAUAACACAU | |
| | UAGAAAGAUAAUGCACUUCACAUAUGUUGAAUGAUUCUUGUAUCCAGGGA | |
| | UAAAUUUCACAUGAAUCAAGUGAGAUUUAGCCCUGGGAUUCAAGAAUGGU | |
| | UCAACAUAUGCAAAUUAAUUGAUGUAAUACAUCAUGUUAAUAUAGCAAAA | |
| | AAAGCACAUAAUUUCAAUUAAUGCAGAGAAAGCAUUUGACAAAAUUCAUC | |
| | AUUUGUUAAUGACACAGACUCUUAACAAAAUAUGUAUAAAAGAAACCUAC | |
| | AUGAAACUAAUAAAGGCCAUAUAAGAAAAGCAGAGAGCUAUAUAGCAUCA | |
| | UAAUCAAUGGAGUGAAACUGAAAGCUUUUUUUUUAAGAUUAGAAACAAAG | |
| | CAAAGAUGUCCACUCUUACCACUUCAUUCAACAUAGUACUGGAAGUUAUA | |
| | GCCAGAAUAAUUGGACAAGAGAAAGAAAUAAAAGUCACUGAAUAAAAAAG | |
| | AAAGUAAAACAUUAUAUGCUUGCGGAUGACGUGAGUGUAUAUGUAGAAAA | |
| | CCAUAAAGACUCAAGAAGAAACUAUGAGAACUAAAUAAAUGAAGUCACUA | |
| | AAGUUGUGGGAUACAAAACCAACAUAGAAAAAUCAGUAGUGAUUUUAUAC | |
| | AUAAACCAUGAGCUACCCAAAAAGGAAAUAAAUAAAAUAAUUCCAUUUAU | |
| | AAUAGUAACAAAAAGAAUAAAAUAAGAUACAAAUUAAACUAAAGAAGUUA | |
| | GAAAAGUGUACAAUGAUAAUUGUAAAACACUGAUGAGGGAAAUUAAUGAA | |
| | UGCACAGAUAAAUGAAAAGACAUCCAGUGUUCAUGGAUUGGCAAAACUAA | |
| | UACUGUCAAAAUGUCCAUCCUAUACAAAGUGAUCUUCAGAUUUAAUGCAA | |
| | UCCCUUAUCAAAAUCCCAGUGGCAUUCUUUACAGAAACAGGAAAACAAAU | |
| | UCUAAAUUUUUAUAGAACCAUGAUAGACCUCUAAUAGCCAACUCAGUCCU | |
| | GAGAAAGAAAAAGCUGAAGGCAUCAUACUUCCUGAUUUCAAAAUGUGUAA | |
| | CCAAAACUACACUAAUAGUGUAUAUGUGCCACAUUUUCUUAAUCCAGUCU | |
| | AUCGUUGUUGGACAUUUAGGUUGGUUCCAAGUCUUUGCUAUUGUGAAUAG | |
| | UGCUCCUAUAAACAUAUGUGUGCAUGUGUCCUUAUAGCAGCAUGAUUUAU | |
| | AAUCCUUUGGGUAUAUACCCAGUAAUGGGAUGGCUGGGUCAAAUGGUAUU | |
| | UCUAGUUCUAGAUCCCUGAAGGAAUCGCCACACUGACUUCCACAAUGGUU | |
| | GAACUAGUUUACGGUCCCACCAACAGUGUAAAAGUGUUCCUAUUUCUCCA | |
| | CAUCCUCUCCAGCACCUGUUGUUUCCUGACUUUUUAAAUGUGACACAUAU | |
| | ACACCAUGGAAUACUAUGCAGCCAUAAAAAAUGGUGAGUUCAUGUCCUUU | |
| | GUAGGGACAUGGAUGAAACUGGAAACCAUCAUUCUCAGCAAACUAUCGCA | |
| | AGGACAAAAAACCAAACACCACAUGUUCUUGCUCAUAGGUGGGAAUUGAA | |
| | CAAUGAGAACACAUGGACACAGGAAGGGGAACAUCACACACCAGGGACUG | |
| | UUGUGGGGUGGGGGGAGUGGGGAGGGAUAGCAUUAGGAGAUAUACCUAAU | |
| | GCUAAAUGAUGAGUUAAUGGGUGCAGCACACCAACAUGGCACAUAUAUAC | |
| | AUAUGUAACAAACCUGUACGUUGUGCACAUGUACCCUAAAAUUUAAAGUA | |
| | UAAUAAUAAUAAAAUAAAAAAACUACACUAAUAGAAAUAGGAUGGUACUG | |
| | GCAUAAAAACAGACAUAUAGAUGAAUGAAAUAGAAAUAGAAGUAAAUGCA | |
| | CACAUCUGUAGCCACCUGAUCUUUGAUAAUGCUGCUGAGAACACACAAUG | |
| | AAGGGAAAGGAUAGUCUUCAAUAAAUGGUAUUAGGAAAACUAGAUAUCCA | |
| | CAUGCAGGGAAAUAAAAUUGUACUACUAUCUCAUAGUAUCUACAAAAACC | |
| | AACUCAAAAUGAAUAAAAGACUUAAACAUACGACAAAAAACUUAAAAACU | |
| | GCUAGAAAAAUACACAGGGAAAAAGGUUCUUGACAUUGAUGUUGGACAGU | |
| | UAUAUUUUGGAUGACACCAGAAUCACAGACAGAAGGAAUGAAAUAGACAA | |
| | AUUGGAUUGUGUCAAACUGAACUUCUGCACAAAGCAAAUAAUCUACAGAG | |
| | UAAAAGACAACCUAUGGAAUGAAGAAAGCAUUUGCAAACCAUAUACUUUA | |
| | UAAGGGGUUAAUAUUCAAAAUACAUAAGAAACUCAGUGACAAAAAAGCAA | |
| | CACUUUUUAAAAAUGAGCAGAGGGUGUGAACAUUUCUUAAAAGAAGACAU | |
| | ACAAAUGUUCAACAAGUGUUGGAAAAGGUGCCCAACAUCACCAAUCAUCA | |
| | GGAAAAUGCAAGUCAAAGCCACACUGAAAUAUCACUUCAAACUUGUUAGA | |
| | AUGAUUAUUAUCAAAAUGAUGAAAAAUAGCAAGUGUUGGCAAGGAUGUGA | |
| | AGAGGGGACCCUUGUGCACUGUUGGUAUGAAUGUAAAUUAGUUCAACUAU | |
| | UAUGAAAAACACUAUGGAAGUUGCUCAAAAAUUUAAAAAUAGAACUACCU | |
| | UAUGAUCCAGCUACCUUAGCUCUAAGUAUAGUUUCAAAAAAUAUAAAAUU | |
| | AUUAUCUGGAAUAGCUUGUUCCAUCCUCAUGUUCAUUAAAGCAUUAUUCA | |
| | CAUUGGCCAAGAUAUGGAAAGAACCUAAAUGUCCUUCAAUCAAUGAAUGG | |
| | ACUACAUAUAAUAUGUACUACUAUAUAUACACACAUAUGUAUGUGUAUAU | |
| | AUGUUUAUGUACUAUAUAUACACAUACAUAUGUGUGUAUAUAUACACACU | |
| | AUAUAUGUAGUUAGAAUAUAUAUACAUGCUAUAUGUAUGUAACUAUAUAU | |
| | AUACACACUUCAUAUACAUGUAGUUAGAAUAUAUAUGCACACACAUUUAA | |
| | ACUACAUAUAUAAAUGUAGUAUAUAGGUAUAUAUGUGUAUUACACAUAUA | |
| | UGUACGUAUAUAUAUAUAAAUGCAGUGUAUAUGUAUAUAUGUAGUGUAUA | |
| | UGUGUAUAUAUAGUAGUAUCUAUAAAAUGUUGUCCAUUCAUCUAUUGAUG | |
| | GACAUUUAGGUUGUUUCUAUAUCUUGCCUACUGUGACUAAUGCUUUAAUG | |
| | AACAUGAGAGUGGAACUAGCUAUUCCAGAUAAUAAUUUUGUAUGUUUUGG | |
| | AUCUAUACCUAGAGCUAAGAUUGCUCUAACAAUGUGUGUGUGUGUGUGUG | |
| | UGUGUGUGUGUGUGUGUAUUCUAACUGCAUACGCACACAAUCCAUGCAAU | |
| | AGGUUAAGAUUCCACCGUAAAAAAGGAAACUUCGUCAUUUGUGACAAGGU | |
| | GGAUGAAUCUGAAGGACUUUAUGUUAAGUGAAAUAAGCCAGACAUAGAAA | |
| | GACAAAUACUGUAUCAUAUCACUUAGAUUUGGAAUCUAAAAUAGUCAAAC | |
| | UCAUAGAAACAGAGAGUAGAAUGGUGGUUGCUAGAGAUUAGGAGGGGGAA | |
| | AAAAUUAGAAAUGUGGGCCAAAGGGUAUAGACUUUCAGAUAUGCAGUAUG | |
| | AAUAAGUUCAGGAGAGCUAAUGUGAAUAUUGAAAUUUCCUAGGAUGGUAU | |
| | CAUGUUUUCACCACAGACAUAGAAGAGAAAAUGGUAACUCUGUGAGAAGA | |
| | UGGACAUGUUAACUAGCUUGUGUAAACCAAAAAUAAAAUUCUAAGCCUCU | |
| | UAACCAACUGAUGGACCCUCCCUCUUGGCCAAAGGGACCCCAAAGAAAUC | |
| | UGUUCAGGCCAUGAUGGGAAGGGGGCAUCUGACAUGCCUUGGUAUAUCCU | |
| | CUUCCCUUUGGAAUUUAGGCACAACUGACCAGCUUUAACAUUAAAUAGAG | |
| | AUCUUAAAACUGACAAAACACUUUUUGUAGCAAUAAAAUACCAAAUUCCA | |
| | ACCUUAUGCUGGUGAAGCAUCACAUGACAGAUAGGCCCUGCAGAGGAUCA | |
| | AAGUAUUUUACCCCUAAAUGUAUUUCUUUGACAUAUUUUGAAAUGGCCCU | |
| | GCAAAGCUAUCUCUUGUCAGGGAAAUUUGCAUUCUGUAGACAAUCUCCUU | |
| | CUCUUAAAAAGUCUUUUCUGGAGAGACUGACAUCUGUUAAGGUCCAAUAA | |
| | GAGACAUUUACCGUCUAUUCUUUCUGAAGCCUGUUGGUGGCUUCAUCUAC | |
| | AAAAAAAAAAAAACAUUGACUUCCUUUCCUUAUUUAACUGAAUUAUUUCU | |
| | UUAUGCUGAGUUUAACCCUUUAGGCAAAGCUUAAUUCUUUCAACCAAUUG | |
| | UCAAUCAGGAAAUUUCUGACUCCACCUAUGACCUGAAAGUAUUUAGUUUG | |
| | AGAUGUCUGCCCUUCCGGGGCUAAUUAAUGUUACAUACCUUACAUGUAUU | |
| | GAUUUAUGCCUUUGCCUGUAACUUCUGUCUCCCUAAAAGGUAUAAAACCA | |
| | AGCUUUAACCCAACCACCUUGGGCGCAUGUUCUCAGGACCUCCUGAGGUG | |
| | GUCACAAACCAUAGUUCUCAUAUUUGGCUCAGAAUAAACCUCUGCAAAUA | |
| | UUUUACAGACUUUGGGUUUUUUGGUCAAUGCUUGAUUGUGAUUAUUAUUU | |
| | CACAGAAUAUAUGUAUGUGAAAACAUCAAGUUGUAUACCUUAAAUAUAUA | |
| | UAAUUUUUAAAAUUUGCCAAUUAUGCCUCAAUAAAUGUGAGGAACAAAAG | |
| | AGGUUACAGUAUGAUGAAUUUAAUAAACUCCUUUUAUAAUAUAUUUAAAU | |
| | AGCAACUUUUGGUGUGUAAAAUGUUGCUAUUUUUACUGAAUAUGCAUGAG | |
| | AAUUUUUCGAUUAAAAUAUUUAUAUAUGUAAUUUGAACACAAAAAUAGAA | |
| | UUUUAACUUUCUUUAGUUUUGAUAUAAAUGCUUUAAAAUAGAAACUGCAA | |
| | CUUUAAAUUUUAAUUCUGAUUUUUUCCAGAAUUUGACUUGAAGUAUUUUA | |
| | AAGAGGAAAAUCUUUUAGCGUAGUUCGAAGUUGGGUAGCAUGAUGUCUCC | |
| | AGCUUUGUUCUUUUUGCUUAAGAUUGUCUUAGCUAUAUGGGCUCUUUUUU | |
| | GGUUCCAUGUGAAUUUUAACAUAGUUUUUUCUAAUUCUGUGAAGAAUGCC | |
| | AAUUGUAGUUUAAUGGGACUAGUGUUGAACCUAUAAAUUACUUUGGACAU | |
| | UAUGGUCAUUUUCACGAUAUUAAUUCUUCCUAUCCAUUAGCAUGGAAUGU | |
| | UUUUCCAUUUUUGUGUGUGUGUGUGUCCUCUCUGAUUUCCUUGAGCAGUG | |
| | AUUUGUAGUUCUCCUUGAAGAGAUAGAUCCUUCACAUCCCUUGUUAGCUG | |
| | UGUACCUAGGUAUUUCAAUCUCUUUGUAGCCAAUUGUGAAUGGGAGUUCA | |
| | GUCAUGAUUUGGCUCUUUGCAUGCCUGUUGUUGGUGAACAGGAAAGCUAG | |
| | CAAUUUUUGCACAUUGAUUUUGUAUGCUGAGACUUUGCUGAAGUUGCUUA | |
| | UCACCUUAAGCUUUUGGGCUGAGACAGUGGGGUUUUCUAGAUAUAGGAAC | |
| | CUGUCAUCUGCAAAGAUAAUUUCACUUCCUCUCUUCCUAUUUGAAUACCC | |
| | UUUAUUUCUUUCUCUUGCUUGAUUGCCCUGGCCAGAACUUCCAAUAAUAU | |
| | AUUGAAUAAAACUGGUGAGAGAGGGCAUCCUUGUCUUGUACUGGUUUUCA | |
| | AGGAGAAUGCUUCCAGCUUUUGCCCAUUCAGUAUGAUAUUGGCUGUCGUC | |
| | UAUGGAUCUUAUUAAUUUUGAAGUAUAUUCCUUCAAUACCUAGUUUAUUG | |
| | AGAAUUUUUAACAUGAAGGGAUACUGAAUUUUAUCAGAGGCCUUUUCUCC | |
| | AUUUUUUGAGAUAAUCAUGUGGUUUCUGUCUUAGUUCUGUUUGUGUGAUU | |
| | AAUUAUAAUUAUUGAUCUGCUUAUGUUGAACCAACCUUGCAUCCAGGGAU | |
| | GAAGCCAACUUGAUUGUGGUGAAUAAGCUUUUUGAUGUGCUGCUGGAUUC | |
| | GGAUUGCCACUAUUUUAUUGAGGAUUUUUGCAUCAGUGUUCAUUAGGGAU | |
| | AUUGGCCUGAAGUUUUGUUGUUGUUGUAUCUCAUCCAGGUUUUGGUAUCA | |
| | GGAUGAUGCUGGUCUCAUAAAAUGAGUUAGGGAGUAGUCCCUCCUUUUCA | |
| | AUUUUUUUGGAAUAGUUCCACUAGAAAUGGUACCAGCUCCUCACACCAGU | |
| | UAGAAUGGCAAUUAUUAAAAAGUCAAGAAAUAACAGAUCCCGGCAAUGCU | |
| | GCGGAGAAAAAGGAAUGCUUUUACACUGUUGUUGGGAAUGAAAUUAGUUC | |
| | AACCAUUUUGGAAGACAGUGUGGUGUUUCUUCAAAGAUCAAGAAGCAGAA | |
| | GUAUCAUUUGAUCCAGCAACCCCAUUACUAGGUAUAUACCCAAAGGAAUA | |
| | UAAAUCAUUCUGUUAUAAAGAUACAUGCCACAUAUGUUCAUCACAGCACC | |
| | AUUCACAAUAGCAAAGACAUGGAAUCAACCCACAUGCUCAUUAAUGAUAG | |
| | ACUGGAUAAAGAAAAUGUGGUACAUAUACCCCAUGGAAUAUAUGCAACUA | |
| | UAAAAAGUAAGAAGAUCAAGUCCUUUGCAGGAACACAGAUGAAGCUGGAG | |
| | GCCAUUAUCCUUAGUGAACUAACACAGGAACAGAAAACUAAACACCGCAU | |
| | GUUCUCACUUAUAAGUGGGAGCUGAAUGAUGAGAACACAUGGACACAGGU | |
| | AGGGGAACAACACACACCGGGGCUUGUUGGGGGCAGUGUGGGGAGGGAGA | |
| | GCCUUAGGAAGAAUAUCUAAUGGAUGCUGAGCUUAAUACCUAGGUGAUAG | |
| | GUUGAUCUGUGCAGCAAACCACCAUGGCACACGUCUACCUAUGUAACAAA | |
| | CCUGUGCAUCCUGCACCCGUCCUUCGAAACUUGAAAUAAAAUUUGAUGAA | |
| | AAAAAAGUAAUCUCACUUCCAUAAGAGGAUGAAAAUGUAUGAGUAGGGCA | |
| | UUAUUUAGGUGAACUGUUCAUAAGAUACCUCAUAACAUCACAUUAUGGCU | |
| | AACAUUAAUUUUUGUUGUUUGGACAGUCUAGCCUGGACAGACUUUGUCUU | |
| | UGUUUUUUGUUAAAAACGAAUCUUGCAAGAAAAACAGAAGAAAAUCUUAA | |
| | AAAUUUUAUAGCAGCUUUAUUUUAAAAUUGAUCGUGUAUGAAUAAAUUAC | |
| | CAUUUUAAAUGUGAAUUCCCUUUUUUAAUGGGAGUUUUAAUAUAAAUGUU | |
| | CAAAAAUAGUUAAUCAUUAUUAGAAGUCAUUUUAAUAUUCUAUUAUUUCA | |
| | AUGUUUCUAGUAUUUACUAAAAUAAACUGCUUCUUUAACAUAAUUUAUUU | |
| | UUCUAUAGAAUUACUAUAUCAGGCUUGCUAUUUGUGCUCAUAAACUUGUC | |
| | AUGAAAUAACUCACAAAAAAGUUUAUAAUCUAAAGAUAGUGACAUAUGAA | |
| | AAUGAUUUUACACUUUUAUAUAUUUAAAAAUCACAACUAUGCACAAUUUC | |
| | UAUUGAAUGGUCAUUAAUGUAUAUACAUGUGUAGAGGGAUAUUAAGAAUU | |
| | CUGAGUGUACAUGGAGAAAAAUUCUCUUUCAGCCAUUUUUCCUGAAUCUA | |
| | AUUUUUGGCAAAAUAACCAUUAAACUAUUUUUUUCUGCCAAAAAUGUGAU | |
| | UUGAUUUUUUUUUUAAAGUAAAAGGAAUGGGGUGUGGUUGGGUGUGGAUA | |
| | UAGAUAUUUGGACAACACAAAUCUUUAAUAAAGAUGAGCAAAGACCUUGA | |
| | AAUGAUGCCAUCCUUUUUUCUUGCUCUUUAUUUCUCCCUAACCAACAGAG | |
| | ACAUUCCAAAAUUCCUCUUACAGUAAACUUACUUUAAACUUAUCUACACA | |
| | GUAAUUAACCUUUUGUUGUUAGCUAUCAGAACAAUGGGUAUUCCUUCACA | |
| | AUGAAAUUUUGAAAAUAAUUACCAGGUCUUGAUAUGGUCUUUUAAAGAGA | |
| | AAUUGAAGUUUCUUUUGGAGAAUUGCUAAGGAAUUAUCUAAAUUGUGUAA | |
| | UUUUUUUUGCUUUCCUUGGUGGUUGGACAUUAAUUCAAACUUUGGCAUUU | |
| | UAUGUGUUUGCCAAAGGAACCUUAUUUAAUGGAUUUGUCUAUGAAUAGAC | |
| | GCUGGAAUAGGCUUGGCUUUCUGGAGAUGCUUGGGACUUUCAGAAAAUGG | |
| | GUAACCAGAAAAAUGGCUUGCUGCCCUUCAAGAAUAAAUAGUGUCCCACU | |
| | GUAAGGUCAAUUUGGCUUCAUCCUCACUCCCUUAUAUACAUGACUAAAUA | |
| | AGGGGAAAGCCUGGGAAACCAUUUCCCAGUUUCCCUUACCAAGAGCGUUU | |
| | CAGGUUAAAUUCUCCUAGUGGGAAGCAGUAGGACUCAAUUUGGAAAUUGA | |
| | AAGAGAAGAAGCUGUUCUCUGGCAGCUCCUAUAAAGGAGGCUACAGUGGG | |
| | CACGUCUUAAUUUCCUGAAUUCUAUCAGCAGUUUCCCUGACUUUUGCUCC | |
| | CUCAGCCCUUCUCAUGGUAUGACCCACUAAUUCCCUAUAUUAAACCCCUU | |
| | UCAACUUGAUACCUAUAAGUUUUAUUUUUUCUUAAUAAAAUUCUGAAUGG | |
| | CAACCCCAAAACUAUUAAAUCCAGGAAUUAUUUCAACAAAUGCAAUGAGA | |
| | AUAUGGUUAAAAAGUUUUUAGCAGACUGUGUGAUAAGAAGAUUGAAUACA | |
| | CACAGAUUCUUUUUCUGUCACCAUUGUCUUUGUUGUGUUUCCCUAGAAGC | |
| | ACACCUGGAGAUUAAAAUUUGAUAAAAAGAUAAUUCCAGGAAGCACUAAU | |
| | AGGGCAAGCAGAAAGUAAUAUAGGGAAGAGAAGGAAGCUAAUACCGGGGG | |
| | AAAAAAAAGCAUGUUAUUGUGGUGGGCUAUUACCUGUUGUAGUUGGUUCA | |
| | UUUAUUUGUUACUAGCAUAAUUCUAACUGAUAUCAUGGGUCAUUGUGAAC | |
| | AAAGGCGUGUUUUUUUGGAAUUAAGGAGCCUAGUCUGCCUGCAAUAGGGA | |
| | GGGGAUUUACAGAUUUUGGAGAGCGCUGAAUUCCAAGGAGUUGGUGUUGA | |
| | CUUGCCAUUUGUCAGAACAUUUCAGAGACAAGCAAAGUGAAGCCCAUUAG | |
| | AUUGAUUACACUCACCUAAUGAAAGAGAAUUUAGAAUUUGAAACAUUGCA | |
| | GAUAUUUAUUACCACUGACCAAAUGUAAUAUCAAGACAGCUGGCAAGUUC | |
| | AAGUGGAUGAGCAUAGCAAUAGAAAUGGCUUGGUCUUUUGGUAAAGACCU | |
| | AAAAGAGGAAUGGCUGUAGAAAGAUACAGAUGUUUGAUGAUAGCUGCAAA | |
| | GUAUACUCAAUGAUUGACAAAGAAAGUAUUUGUUAUAAUUUUCUCAGAUC | |
| | CAAGUGGAUCUGUAUAAAGAUCUAAAUAGUACAGUUUUCUAUAUCAGGAU | |
| | AGUUUGAUGCCAUUAUUGAUUAGUAGCUGCUAUCUUUUUUUCCUGUUUUU | |
| | GUUUUCUUAAUCUUCUUUUGUAUAAAAUCUUAAUUAAUGUCUACAUUUUU | |
| | GGCAUGUUGGCUGGCUGGCAUAUUUAGCUAAUCCAGCAUAUUCCUUGGUG | |
| | GCCUUAUUGGGGAAAAGUGUUAGUUUUUCAAAGUUUUUUUUUCCUAUUUA | |
| | CUGUUUCCAAAAAGGUUUUAAGUAAAUUCCAUACUUCUUUUUCUAACUAG | |
| | UGGCAGUUGAUUCUAAUAAAUGUAAGCAAACCAUAUUGUAAAAGAUAGCA | |
| | AUUUGAUAUCACAUACAUACAAAUUAAAUGCAAAAUACAACAGCCCAAGA | |
| | AUCCCCAAAAAGAUAUGCGACUGCACAGAAACAAGAAGCACAGCCCUGUG | |
| | CUAGUUAUGAACCACUUUUGGAUUCCUUGUGUUGACAUGGAGCAGAGCCG | |
| | UGGGGACAUGGAUUUUGGAGGAGCUGACCACUUUGGAAUAGUAGCAAGGA | |
| | AGAAGUAUAUACCUAUUUCUAGAUAAAAUUCAGAAGGCUUUUUUCUGACU | |
| | UUUGAUGACAGUUUGAUUAUUGCAGUCUACAAUGAAGAUUCAUGAAAUUG | |
| | AGUUCUCAAAGAUUUCAAGAAUAAAACAACCUAUACCGUUUGAAUAUUUG | |
| | AUCAGUUUUAAAUAUUUGGGAUUUGAAAGGCAAAACUAUGUAAAUUUCCA | |
| | UAUGCUAUCACUAAAGGCUGGUAUUUUAUUUAAUAAACUUUGUCAAGAGA | |
| | CUAACUUUUCAAAACUCAUUCUUCUUUUUUACGAGAAGAAGUGAAAGGAG | |
| | UAGAAAUACAGCCUGUUGAUGCCCACAAACUUAUCCUAAGAGUUUACACA | |
| | UAUCUUGUUAAGCUUCUCAACAAUGCUAUAAAUUGUGGGUUUUGUCCCAG | |
| | CUUUACAAAAGAGGGUCAUGAGGCGAAGAUAAUUUCUUUAGAAACUUGAU | |
| | UAAGAUCAUGAAAAUAAAUGUGAUAGAAUUAGGAUUCAAAUCCAGGUUUA | |
| | UAUUACUGAUUGAUUCAUCCAAAAUGUUUAUUUACUAUGUGCCAAAUCUU | |
| | AAGCUAAAUAUUGUAUUAGUUUGCUAGUGUAUCCAUAGAAAAAUGCCACA | |
| | GACUGUAUAGCUUAAAAAACAGAAAUAUAUUUUCCCACAAUUCUGGAGAA | |
| | UAGAAGUCUGAGAUCAAGAUGCCAGCUGGGUUAGUUUCUUCUGAGGCUUU | |
| | CAGAAGAUGGUUAAGCCAAACUGUUAAUUUUUAAACAUCAUGAACUAACU | |
| | UGUCCUCAAAGAACUUCAUCAACUUGAACAUUUUCAAAGAGCUCUAUAAG | |
| | GCAGCUCAGCAUGGCAGUUUUUUUACUGAAAUAUCUUAUCUGGAAGAUGG | |
| | CAAAACAGACCUGGACCUUCACAAGCCAAUGGUUGCUUGUAUUCAUAUCA | |
| | CAGUUCACUUGGGUAAGUGGUAACAACAGAAUAAAAAGCAGAUUGCCUCC | |
| | AUACUGGUUGGGUGAGAUAGCUUCAUUUUUGGAAAAUCAAUUGAAUCAUG | |
| | AAAAUCUUCCCAAUGGUAUAAUUUGUUCCGGAGUUCUUUUGAUAGUUAAG | |
| | AAGGGAAAUAGUAAUCAUUGUCCACAGUCUUUAUUUAUUUCUUUUUUUUA | |
| | CAAAUACUCUUAUGGCAUUCUCGAGCCAUCAUUCUAUGGUGUGGUGACUG | |
| | AUUGUACUCCAUGCAGGUCACCUCCUUGCCAUGUCUUCCCAUGGUGUUCC | |
| | CUCUGUGUGUGCACAUCUGUGUGCUAGUCUCCUUUUCUUUUAAGGACAUC | |
| | AUUUAUAUUGGAUUAGAGCCCACCCUAAUGAUGUGACUUUAACUUAAUUA | |
| | CCUUUUUAAAGACUAUCUCCAAAUGUAGUCACUUUCUGAGGUAUGUCAAC | |
| | CUAUUAAUUCUGGGUACAAAGUUCGGCCCAUAAAUGAUGUUAUGGACCCA | |
| | AUGUCUAACAAAGGCCAAACACUCUCCCUGACUUCGGAGAAUUUAGUUUA | |
| | GUAAGGGAGAUUAGCAUUAAGCAAAAAAUUACCUAUAUAUAUAUAUGUAC | |
| | AUUGCUCUAGUAAUAAGAACUCUGAAGGGGAAUUAAAGGAUGUUACAAGA | |
| | GUUUGAACACAUCCAUGGGGAUAGCAAAGACUUUCUUGAGGAAAUAAUGA | |
| | CCCAGUUAACUCUGACAAUGAAAAGGGCAGUGAAGUCUGGUCCAGGCAGA | |
| | UAAUCUACUUGUGCAGGGAAAGCUCCUAUGCGGAGGGAGCUUAGGGAAUU | |
| | UAAGGUUAGAAGUGAGAGAAGCCUUGUCAGCUUUUGGUUAGAAAUUUGGU | |
| | CUUUAUUCCAAGAGUAAUUAGAAGCAAUUGAAAGUUUCAUAGUUGAUUUG | |
| | UGUUUUGAAAAGAGUACUCUUUCUGCUUAUAAAAUACUCAGCUGAUUAAC | |
| | CACAUUUUUUAUUGACUUUUCAAUUGGUGGUGGUUCUAUCAUGCACACGC | |
| | AGAAAAAUUUAAAAGUUAGCUUAACUUUUUAGGACUCUCAUUACUUCCUA | |
| | UUUUCUAGCUUGAAGUUUGCAAGAAGUGAAAAAAAAGAAUUAUAAAAUUU | |
| | UACUUCAACCUUUGAAAGGAAGAUUCCAUUAUUUAGACAAUUGGAAAAGA | |
| | AGACAAAAGACUAAAAACAUUGUUGUUAAUCAGGAUCUGCCUUAGAGUUA | |
| | AAUCUUUCUUUGGUAUUUCCUGUCUGUCUCCACAAAGUUGUCAGAUAUUU | |
| | GGACCUAUUCUUCAUACGAUGAACAUUACUAACUGCCUUUAGUCCCAGUA | |
| | GGAACUAUGUGCUUGCACUGAAGAAAAAAAAUUGUCUGAAAGGCUAAAUA | |
| | CAAAUAUAGUAAGGUAUAUAUGUAUAGAAGUUGGUGAUUUAUCGGGGCAC | |
| | CUGCCCUGAUAUUCACGUAGGUUCUUUUCUAUUUUCCUUAAGUGUCAGCC | |
| | AGCUUGAGAAAUAAAGGGACAGAGUACAAAAGAAAGAAAUUUUAAAGCUG | |
| | GGCAUCUGGGGGAGACAUCACAUGUCGGUAGGUUCUGUGAUGCCACACAA | |
| | GCCGCAAAAACCAGCAAGUUUUUAUUAGGGAGUUUCAAGAGGGGAGGGAG | |
| | UGUGCGAACAGGUGUGGGUCACAGACAUCAAGUCCUUUACAAGGUAAUAG | |
| | AAUAUCACAAGGCAAAUGGAGGCAGGGCGAGAUCACAGGACCACAGGACC | |
| | CAGGGGAAAUGAAAAUUGCUAAUGAAGUUUUGGGCACCAUUGUUAUUGAU | |
| | AGCAUCUUAUCAGGAGACAGGUUUUGAGAUCAACUGGUCUGACCAAAAUU | |
| | UAUUAGGCGGGAAUUUCCUCUUCCUAAUAAGCCUGGGAGCGCUAUGGGAG | |
| | ACUGGAGUCUAUUUCACCUCUGCACUCUCGACCAUAAGAGAUGACCACGC | |
| | CCAGGGGAGCCAGUUCAGAGACCUACCCCCAGGUGGCAUUCUCUUUCUCA | |
| | GGGAUGUUCCAUGCUGAGAAAAAAAAUUCAGCGAUAUUUCUCCCAUUUGC | |
| | UUUUGAAAGAAGAGAAAUAUGGCUCUGUUCCACCCGGCUCACCGGCGGUC | |
| | AGAGUUUAAGGUUAUCUCUCUUAUUCCCUGAACAAUUGCUGUUAUCCUGU | |
| | UCUUUUUUCAAGGUGCCCAGAUUUCAUAUUGCUCAAACACACAUGCCGUA | |
| | CAAUUUGUGCAGUUAAUGCAAUUAUUACAGGGUCCUGAGGUGAUAUACAU | |
| | CCUCCUCAGCUGACAGGAUUAAGAGAUUAAAGUAAAGACAGGCAUAGGAA | |
| | AUCACAAGGGUAUUGAUUGGGGAAGUGAUAAGUGUCCAUGAAAUCUCCAU | |
| | AAUUUAUGUUUAGAGAUUGCGGUAAAGACAGGCAUAAGAAAUUAUAAAAG | |
| | UAUUAAUUUGGGGUACUAAUAAAUAUCCAUGAAAUCUUCACAAUCCACAU | |
| | UCUUCUGCCAUGGCUUCAGUGGGUCCCUCCAUUUGGGGUCCCUGACUCCC | |
| | GCAACAGUGAUUAAAAUAUUAAGUGCUUCCAAAUGGUACAAUUUGAGAUU | |
| | CUGUGUAUAAAAUUGAGUAUGAAUGAUUAAAAUAGUGAAGUAGUUUCCAU | |
| | CGACUUCUGAAAACAAAGAAGCUUUCUGUUUGUUCAGUUUCUUUUUUAAU | |
| | UUUAAAAAUUUAUGACUAUUUUUAUUACAUAAUAUUCAAUCUUAUAAUUG | |
| | ACAUUGAAUGUAUUUUUUUCAAAAUAAAAUUUUUCUUCACCUUCAAAAAA | |
| | GACUUCUAUUUUACUAAAAUGAUGUAGAAACCAGUUAGCCUUAAGUUUAA | |
| | AUGGAGACCAAUAUAUUAAUUUUCAAGUUACAGAAUUCCUACCAAAGAGG | |
| | GGAUAGCUAUAGCUAUCUAACACCCUGUGAGAAAUAGAAUGAUAACAGUA | |
| | AAUAUCAGCCAUAACAAAGUUAUCAAAUUCCUCUUGUUUCAUAGGAGUAC | |
| | AAAGAAAUCAUGCUUUAUCAUCAAAUUCCUCUUGUUUCUUAGGAGUACAA | |
| | AGAAAUAUGUUAAAGUAUGAUAUUAAUAGAACAUUAAUUGGUAAAAGUCA | |
| | CAAUCUGUAUUUUGUAGAUCUGUACAAUGAAAAAAUGAAUGACUUAAUGU | |
| | AGACAGGUGAAUAGUUUUAGGAAAUGAUUAGACUUUCAAUAAAACACAAC | |
| | GUUGCAUAGCUAGCAUAAAGCUAGUUAAAAUAUGAGAAAUCAAAAGCUGC | |
| | UUUCCUGAGGCUAGCCAAAUGACGAGAAGUGAGGUAAGUCCAGAGUCUGA | |
| | CCCAAAUUAAAAUCUGUUUCCAGACUCAAUCUGAAUGCUUCCUUUUUCCU | |
| | UUAUUGCUCAGAGUAGAUUUUAUAAGUAUUGCCUGGAAGAAGAAGCUAUA | |
| | UUAUAUAUUAAAAUAUAUGCUAUUCUUUCUUUAAAAAUUCAAAUAAACAU | |
| | AUUUAGUUCACAUCAUUACUCAUGUUUGUGUUCAAGGGUAGAUAAAAGAG | |
| | GAGUAGGGAAAGUAGUACUCACUUGAGCUGAUAAAUAUCUCUAAGAAAAU | |
| | UUUAAAAUACAUGUUUAAAUUUAUGUGUAAGUUUUAUUAAUUUUUAUUUG | |
| | ACAUUUAAAUGUAAAAUAUUUUAUCACUCAAAUAUAUUUCAGCUAUAAAA | |
| | UAAAUAACAGUUAAGGAUUUAAAAAUAUUUAUACUCUGAACUGUAGAAAU | |
| | AUUAGUCCCUUGAAAGAUCUCUUUAUUUCCAAAAUAUUUCUAUUGCAAGU | |
| | GUCAGAAAUGGUAACUAUAUAACAAUUGAGAACGUAGGAAAUUAUUUAUU | |
| | CAUAUAUGAAUUCCCACUCAUAAAUUUUGGCUGCCUUCACUGAAGGCUUU | |
| | CACCAAAGUGCUAUCAAUUUUCACUUCUCCUUCAUUCCCAUGAUGUCUUG | |
| | CAGAAACAAAAGGAGGAAGAAAAAAUAAUCUAAAGAUUGAAUGGCUGUCC | |
| | CACAGAUUCACAGCCAAAAUUAAAACAAGAACAUUUUUGGCCUACUUAAU | |
| | AUACUUACUUUCCCCAAAAAGAAUCUCAUAAAAAUUGGUACCAAUUAUUG | |
| | UUACACAUAAAAAUGCAGACCUAAUUUCCUUUACAUUUGCUGGCACAUAC | |
| | CUAGUCUUGUAAGAAAGUGAAAAUAGUAUGCUUAUACUGUGUAUACAUGU | |
| | GGAGCUCCCUCCACACAGGAGCUUUCCCUGCACAUAUAGCUCUUUGUACA | |
| | GUCUAAAGUGCACCACCACUUUACUAAUAAUAUUUUUGUUGUAAUGUUAA | |
| | UUUUAAAUUUUAAUAUUUAACUUUCAAUAAGAUCAAAAUAAUAUCCUAGA | |
| | GUUCUGUGGAAGAAGAGAAAAAAUUAAUCAGCACAGCUAUUUUUAUAUAU | |
| | UUAAAGCAAGUUACCAGUGACAUACACAGUCAUCUGUCUAAUGUGUGUUC | |
| | UCAGGGGAUGUUUUUAUUUGCUCCAUUUUAUUUGUCCUACAUAAAACAAA | |
| | UACUGUUAAGUAAAAGAAUAUAAGAAACACAUGAAUAAAUUUAGACCUUU | |
| | CUUUUAAGAGUGAUUAAAAUGUUUCUCUUAGAAGAGUAGUAUUUACUUCU | |
| | UUAGGUAACUAAUGACUAAUAUUUUUGCUUUCUCCAGGUAACUAGUGAAA | |
| | GUGUUUAAAUAUCUAGAGUUAGAACCAAAAGACAGGAAAUUAUUUAAGUU | |
| | GUAAAAUAAUACUAUAAUUGAUAUAUUAUACCAAAAUCAAUUACAAUCUC | |
| | CCAUAGGAUCAGAAACCAUGAAAAAUUAUGUAUUUCAACAAUGGGAACAU | |
| | UUUUAAACUGAAGAAAAUAAUUUUAAUAAAACCUUAUUAAUAAUUUUAAU | |
| | AAAAAUACACCUAGCUCAAAACCUUUCCCUAGUAACACUUUAGUAUGUGU | |
| | ACUGAACUGUUCAUCUUAAGAUAAUGGUAAGAAAUUUUUAUAUGCUGUGU | |
| | UUCAGUCACAAAAAGAGGAAAUGCAUAAAUUCAUACAAUUCAUUUUUCUA | |
| | CUAAACUUAAACUUUUAGUUCCAAAAUGGAAAAAUAGCUAAUUUACUGAA | |
| | AAUCUGUUAUAAAUACUAUUUGAUAAUUAUUUAAAACUAAAACUUCUAAC | |
| | UCUAUAUUUACCUUCACUAGGAUAUUCUAUGCUUUCUACGUCAUUGCUUA | |
| | UGAGUCUCAUGUUUUGAUAAGACAAAAUUGUGUAAGUUAAAAGAUUAUCU | |
| | UUGUAUCAUGGAAAGAUAAUGGUAUCUAAAUUUGGAAAGAUAUAUUUUAA | |
| | CUGAAAAAUGUGCUCAACCAGAAUUAACAAAAUAUAUGAAAUCCAGGUCU | |
| | ACCAUUUACUAACUCUGUAAUAAAGUUUUUAAUUUCUUUGAGUUUCAAAU | |
| | UUCUCACCUAUAAAAUGGCAAUGAUAAAAAGCAAUACCAUAAGGCUACUG | |
| | UGAAAAUUAUAAGAGACUUAGAAAGUGCAAUGCAUAAUGUUUGAUUUAUU | |
| | GUAGUUAUUAUUUAAAUUUUUAACAGUAUGUAUAUAUAUGUAAUAUUGGA | |
| | UUUUAUUUUAUUAAUUCGAUUAAGACAUAUUAUGUAGAAAAAUAAACUUC | |
| | CUAUGCCAUAUAUAACACAAAUGUAUUAAGAAACAAUUGUGCUAAAUUAA | |
| | GUGAAGUUAAAAAGCGUAUGAUUCUUCAUAACAAAACUAUAUACAAUCAU | |
| | UUCAAGAACAGUCAAAACAUAUGCUAGGCAAUAGAGUGCUGAAAAAUGUU | |
| | UGGUAUCAUCUCUUUGAGAAGUGAGGUAAUUUUUCGCUUCUUAAAACAUA | |
| | AAGAUGGAUUUAGGAGUAAUAUAUAUUCUUUUCUAUAUGGGUAGAUAUAG | |
| | ACCAGUAAAAGUGAAUCUAUGCAUUGAGGUAAUUACAUGUACAUUGAAAA | |
| | AAAAUGAAGCUAAUUUAAGAAAGUCUUCUCUCAUCUAGAUAUUUUAGCUC | |
| | AAAUGCCUCAGGAAUAAAUUAUCUCAUAUAAGCAAAUCACUUAUUCUCUU | |
| | GUCUUUACUAAUAUAGUUUUUGCUUAUUGUUUUUAUGACUCUUGAAUUAC | |
| | UAUUGAGAUAAUAGAUAAUGCACAUGCCUUAAUCUAUAUUUCAAAGAAAA | |
| | AUUUAAAAUAUUUUAAAUUCCAUGUUCUUCUUGUAGUUUCUUAAUCAAGU | |
| | AUUUUGCCUAUUUCUCAAACAAAAUGGUAAUUAAAAAAAACACCUGUAAU | |
| | UUCCAGUUUCUAUUUUUCACUUUUCCCCUUGCUUAUUUAGAGCUGACUCU | |
| | GACCAAAUCUGUGAAGCAGUGGGUUUGUGUUUUCUUCCUGGUUUCCUUAU | |
| | GUAUAGAUGACAGUAUCUAUCCCUGGGCAGUGCCAGGGCAUGUCCUUAGA | |
| | GCGGUUUUUUGAGUAGUAAUGAUGGAUUUACAUUGAUUCAUCUAUACUCU | |
| | UGGCCUAGUUUCUUUCGUGGCCAUCAUCCUGCCUUCCUGGAAUCCAGACC | |
| | GAAAUUAGUGGUUCUGGUUUUGCCUCAGAACUGAGUAAAAUUAGCUAUGC | |
| | UAGCUGGAGACUAGCUAAACUGGCUCCAAGUAAACCAAAAGUAGAAUUGC | |
| | CCUUCUCCCUUCUCCAACCUUCUCCUUCCUAACCUUUCCAACAAAUCAUA | |
| | GAUGUGUCUAAGCUCUUAGAGUUCUAGGGAAGUUACCUGUAUUUAAUCUU | |
| | GUCCUUUCUCUACAGAUAAGACUCUAGGAGACUAAGACUUAAGGAAAAAA | |
| | UCUCUAAGUGAUGUGUGUUUUAAAAAGAAAGCCCAGGAAUUAUAAACUGU | |
| | ACACAAAUAAAGGACAUUGUAUUUUUAACGUUCUCUACUUCAGUUGUACA | |
| | CUGUCCUACUAAAAGUGAUGAUAUCUUCAAUAUUCUGCUAGAGCCACUUC | |
| | UCACUUCAUGUUGCCUUGUAUAUUUUGCCUUUAAAAUAUUCAUAAACCUU | |
| | UCAAUUGUUUAACUUUUACAUUUUCGGACAACCUAGUCAUCAGAGAACUC | |
| | AUAUUUCUGAGUUCCUUGCUUAAACAAUAUUUAGCCAGCUGAAGGUUUCA | |
| | AUUAAGAGACUUUAUAUUAGCCUCAGAUCAACAGUUCCUAAGAAACAAUA | |
| | GAAGCUUUAUCAAAUAAUCCACUUAUCAUCUCUGACAAAAGAACAUAUAU | |
| | AUUGUCUUUCCUAAAAAUAUAGGAUUGUUGUAAUAUGUCAAUCUCUCUAU | |
| | UUUCCUAAUAUGUUCAUUAGUUCACUCUUCAUAUCCCAGGGGAUAAAAUG | |
| | UCUUGAUUACCUGAUUAGGAAAUAUAUUUGCUCAUUAUUCCCUGAAAACA | |
| | UUUUGCACUGUGUAUAUUUAAUGCUGUUUAUACAAAAGAAAGGCCUAUUU | |
| | UUUUUUUAAUCUACUCCAACAAUAUGUUUGUUCUUAAAUCUUCUGGCAUA | |
| | ACAUUCUGUAUUUUCCCUGCCCCAGACAUGAAGUUAACCACUUCUCGAAG | |
| | GCUCCUUUUGUUGAAGGAAGGUAGUUAGAAGUAAAGAUCUGAGGAGUAGA | |
| | UUUGCUAGUUGGUGUUGCUGUCCACCAUUUUGGUGGACAAAGCUAAAUUU | |
| | AUUUUUAUGUCUAUCUAUGUGUUUGUUGAAAUGCAUGAGUUCAUACUGAU | |
| | AUACCCAAUUCCAAGAUGACAUCCCAGGGUUCGUCCUAGUUUUCUCUCUU | |
| | UCCAAAUUUUUAACUCCUUUCAACUACAGUAAUAAUCCUGACUACAAUUA | |
| | UUCUUAAUAUAUAUACUUAUUUGAUCAAUCCCCUCCAUGUAACCAGUCUU | |
| | CCAUUGCCACUGCUGCCCCCUUCCCUGCAUAGAUACCCUCUCCAUCUUGC | |
| | UCUGGGCUCUAACCCCACAGACCUUGCUAUCUUUCUCUAUAUGAGCGCUU | |
| | UCCUCAUUACACUUGUGCCCCAAUAGCUCACACUGGGCAGCCCUCCAAUA | |
| | CAGACAGCUAUCUCAUUCUGCUCAGGUUUUGAGACCACUUUGAGAGCACU | |
| | GAUGCUUCCCUGCCUUGCUUGGCAUCACCUAACGGUUUUGGGUCUGAAUU | |
| | GCCCGGAAAAGAAAGGGAACAGGAAGAGAAAGAGUAAACUUUGUAUGUUA | |
| | AAGAUUCAAGUUAAUGAAAAAGAUCCUACCAUUUGACAUUCCAUGGCACC | |
| | CUUUGGGAUGAGUUGCAUGUCCUCUCUUACUAAUAUUGUACAGUUUAGGC | |
| | AUGUUUUCUUCUUUUCUCUUAAAACAUUUUUCUCUCUUGGUUGCUAAGGC | |
| | CACAUCCUGUUGUGAUUAUUUUUAUUUAUUAUUGUUUUUUUUUCUAAUCC | |
| | UCUUCAUUGAUUUCCUUUCAUUUGGUUUUAAAACAUAGUUUUAAAUUUUA | |
| | UUUUAGAUUCAAGGGGUACAUGUGCAGGUUUGUUACAUGAGUAAAUUGCA | |
| | UGAUUGCUGAGAUGAAUGAUCCCAUCCACACAGGGAGUGAGCAUAGUACC | |
| | CAACAGGUAGUGAUAUGGUUUGCCUGUGUCCCCACCUGUAUCUCAUGUUG | |
| | AAUUGUAGCUCCCAUAAUUCUCACAUGUUGUGGGAGGAACCCGAUGGGAG | |
| | AUAAUUGAAUCAUGGAGGUGGUUU | |
| 2063 | CAAACCUGCACAUCCUGCACAUGUACCCCGGAACUGAAAAUUAAAAAAAA | RNA |
| | UUCCUUACUACUAUUUAAAAAAACUUUAUUAAAGUACAAUCAGUGUGCAA | sequence |
| | AAAAGUAUACCUAUCAUAAGAGGGCAUGUUGAUAAAAUUUCACAAAUUGG | of |
| | ACACAACCACAAAACCAGGAACAAAAUCAAGAAGCAAAACUUUACUAGUG | XLOC_ |
| | UCUCAGAAGCUCUUCUUAUGCACCCCCCGAGCCCCUGCCCCAUCACUACC | 005184 |
| | UCCUCCAUACCUCCAACAACAUCUAUUAGUUUUGUCUGUUUUUGAACUUU | |
| | ACAUAAUGGAAUCACAUAAUGUGUAAUAUUUUGCAUUUGACUUAUUUAUU | |
| | GCCCUCAACAUUAGGUUUAAGAGGUGUGCUUAAUUUUUUAAAAUUUAUUG | |
| | UGUGCUGCCACAAUAAAUUAAAUGUCAUUGAAACAUUAUUUGUAGGCACA | |
| | GUUUAAGGUAUCAGAAUAAGGUUCACCAUAGAGAAUUUGCAUUUGAUUCU | |
| | AUCAGGUCUCUAGGGGCACUGGGGCAUUGGUAGAUUGAUCAGAGGGUUUC | |
| | UCAAUCAUCUAGCUGAAACAUGAUCAUAAUCUGUUAAGGUCCACUGCCUA | |
| | UGACCCCACGCCCUCAACCCCUGGCCCUGCUUGAAUUAUCACUGAGGCUA | |
| | CCCUUCUGGCAGUCUCCUGGAUGUAGAGUAGGGGUGUGUUUACUUCUAUU | |
| | UUACCUUUGCAAUGAGAAAAUAGCCCUUUGAAGUUCUGGUUUAAGUUGGG | |
| | AAGACUCUCCUUUUGAAUGCCUCACCCUGGCAUGGCUGGGCUUUGAUUAC | |
| | AGUUCCCCCUUGACCCUCAAGGACAUCAGACUAAUAUUAACUUUUACUCU | |
| | GUGUGGUAAAUAUCCUCAGGGCAAAAGCAACUUUGACGUCCAUUAUGCAC | |
| | CUUUGGUGAGAAAAGUCCCCUUACACUUUGACUUGCUAUUUCCUUACUAA | |
| | CUUGGCAUUUCUUUAGUAGCAUCAAGAAAAUAAGAUGAUAGUUUUCCUUA | |
| | UUUUUAAAGCAAAGGUUUUGUUGAGAUAACCUUGUGAACAAUAUUACAGA | |
| | AAACUUUCAUAGCUACUUGCUAUGGAGAUCUUAGGUCCUUUCAUCCCACA | |
| | CUUGUUUUGGCUAUAAAAUUGCCUUUAGUACAUUCUUAAACUGAAGCUUC | |
| | UGAUGAUAAUUCUUAAGUUUGUGCUCCUAAGGGCCUACUGGGACCUAUUU | |
| | CUUUGACUUUUUCUCCCGAAGACACUAUCUUUCCCCAGUAUCUGAAUGUU | |
| | UUUGGCUUUGAGUAAAAUGUUUGCUUAUUUUAUGUAGUAGAUAUUAGUGU | |
| | CUUUACUGUUUUAUCUCCCUUUCAGAAAGGAACAUCUGAGUUUGCAAUAG | |
| | UUUAAACUGAAUCUUGUUUUACUGUCUUUUCUUCCUUUGUACUUGUAGGC | |
| | CUACAUAGAAUUGAUUACUUCACUUCUUUUGCUUUCUCUUGCUACUUGAU | |
| | CUAGGUAUUGUUAAUUAUCCACGGAUGAGGAUAAAUAACAAAUACACACA | |
| | CACACACACACCCCUUAGGCAUAUCAUAUUCAAACUUCAGAAAACCAAAG | |
| | ACAAAGAAAAAUCUCUAAAGAAAUCAAAUAAUAAAAACAGACAUAUAGAG | |
| | GAACAAGAAUAAGAAUUAUACUGGACUGUUUGUCAGAAACCAUGAAAGCA | |
| | AGAAGAGAGUGGAGUACAUUAUUUAAAGUGUUGAAAUAAAAACUUUCACC | |
| | AGGCUAGAAUUUUAUAACCAGCAAAAUUAUCCUUCAAAUGUGAAGGAGAG | |
| | AGGGAGACUUUUUCAGAGAAACAAAACUGAGGAAACUCAUUGUCAGUAGA | |
| | CCUGAUUUAUAAGACCAUGUUAAAAGAAGUUAUUCAUUAAGAAGAAAAAU | |
| | GAUAUAGGGCACAGAUCUACACAAAGAAGAGUGUCAGAGGAGAAAUAAAU | |
| | GAAGAUAAGAUAACAUUGUUUAGUUUUUUCAAGCUAAACUUUCUUAACUA | |
| | UAUAAAACAUAACUGUUUCAAGCAAUACCAACAAUGCACUGGGUGGUUAU | |
| | AGUAUAGGGGUAAGGGAAAUGAAAGACAACAGUGUGAAAUGAACAAGCAG | |
| | GAGCAAAUGGGAAUAUUCUGUUUUAAGAUAUAUAUACUACAUGUGAAGGC | |
| | AGACUUAGGUUACUUAAAAUAUAAAUAUGUAUUAUAUAUAAUAUAUGUAA | |
| | AUAUAUAUAAUAUGUAUUAUAUAUAUAAUAUAUGUAAAUAUAUAUAAUAU | |
| | GUAUUAUAUAUAUAAUAUAUGUAAAUAUAUAUAAUAUAUAUUAUCUUUAU | |
| | CACAUCUGGUACAGCAGCUGCAAUUGGAGUCAUCACUUUGUUAAACUUAU | |
| | GAUAAGGCACUGUCAUUCUCCAAGAUCCAUCUGUCUUCUGCACAGCCAAA | |
| | UGGGAGAGUUGAAUGGGAAUAACCACUCGUGCUUCUUUCAAGUCCUUGAU | |
| | AGUGGCACUAAUCUCCACAGUCCCUCCAGGGAUGCGAUAUUGUUUUUGAU | |
| | UUACUAUUUUUCUAGGUAGAGGCAGCUCUAAUGGCUUCCAUUUGGCCUUU | |
| | CUCACCGUAACAGCCCUCACCCUACCAGUCAGGGAGCCAAUGUGGGGGUU | |
| | CUGCCAGUUGCUAAGAAUGUCUAUGCCUAUUAUGCACUGGGGAAAUGACC | |
| | ACAGGAUGAGUCCGGGAACCCACUGGAUCCACUCUUUGUUGGACCUGAGC | |
| | UACAACUCCAUUAAUUACCUGACCUCCAUAAGCCCCUGCUUUAACUGGAG | |
| | GACCACAGUGACAUUUUGGGUCCUCUGGAAUCAAUGUCAGCUCAGAGCCA | |
| | GUGUCCACUAGUUCCUUAAAUGUCGAUCAUUUCCCUCUCCCCAGUGGGCA | |
| | GGUACCCUGGUAAAAGGCUGGAGGUCUCCUUGGGGAAGGAUGGGAGAAAG | |
| | AUUCACUGCUCAAAUUGUCGAUAACGUAGUGAGGUCCUUCCUCAAGGGUA | |
| | CCUGGCCUCCCCUUCAUUCAAGGAGUUCUGGGUCUGUAAACUGGCUCAAG | |
| | UCUGGAAAUCGAUUGAGGGGCCAUUAUUCUCUGUUUUUAUAAUUUAAAUU | |
| | AGUCUUUUGUCCAUUCGACCUAGAAGUUUUCUGCUAGUAUAAAUUAAGUA | |
| | CGAAUGCAGUAGGCUUCCUAUCAAUUUCACUUCUAGGAACACCGUGAUUA | |
| | AUUAGCCAAUGCCAUAACUCUACACGAGUCAGACCAUUCUGAUUGCCACU | |
| | UUGCCUCUGCUGUCCAAUAAGGUAGCUACGCCCACCUUGCCUUUGAAGGU | |
| | UGAGUGCCACCACUUGGCUCCUGCCACCUCAGGAUCUAAUUACUCCCAUU | |
| | GUAUUUAAAUUUUGUGGUUGAGUGACUGCAGUUCCCACUGUUAGAUCUGA | |
| | CAUACAGAGAAGAGCAAUUACAGGGCUCUUCAAAGAUGCAGGUGCCACCC | |
| | UCGCAAAUCUAUUUUGCAAGGCAUUGGUCAAGGGUGUAUCUUCUGGACCC | |
| | UCCCAGCUGGGAUGAGAGGUCUAAAGUGACUAAUCCACUCCAUCAUCCCA | |
| | AUCUCCCUAAGCCUCUGGAUCCCUUCCUCUCCAUUAAACCAAGGGAGAUC | |
| | AGGCAUUUCCAGCUUGCUUACAGUGGGCCAUCUUUUAGUCCAUAUUUCAG | |
| | CUAACCAAGCAAAUAAACUAUUAGAACAUUUUUUAACUCACCAAGCUGCA | |
| | ACAUUAGAAGCAGAGUUCCUAUGUAGUGGGCCCAAAUUAAUAAAUUCGGC | |
| | CUGAUCCAACUCUGUAUUCCUUCCACCAUUAUCCCACACCCUUAAUAUCC | |
| | AUUCCCAUGUUCUUCAGAUUUCUGUUUAUAUAAAUGAGAAAACUCAAGCA | |
| | GUUCUUUUCCAGUGUAGCGCACCUCCUCAUGGGUCACACUCUCAACCUUA | |
| | CUUCUAGGGGCCCGCCGGGACUUUAGUCUAGUUAUAGGUCUAGAAGCAAA | |
| | CAGGCAGGUUGGGGGUGGCUCCUGAGGAGAAUCAGCAUUACCUUGCCUGG | |
| | CAACCACCUCAGGGGAGGCCAUCACUGUUGCCUCAGGCGGCACAGGGUUU | |
| | AUCUCUUCAGACAAAGGUAGAAAGGCUGAUGGCAGCAUGGGUCGGGGAGG | |
| | GGAUGUUGCCACUACUGGGGAUGGGGAAGCUGUUCCUUCUGGCAAAAAAG | |
| | AUUCAUCAGAGUUUACAAAUUCAGUAUCCCCAGCUUCAUCAGUGUCUUCC | |
| | CACAUGUUCCUGUUCCAAGUUGCAGGGUCCCAUUCUUUUCCAAUCAAUCU | |
| | CACUUUAAUAGACACCUGGUGAGGCUGUGCAUGCACCUUCUGUUGCAGGU | |
| | CACCCACUUUCAUGAUAAGAGUUUGUGCCUGUUUUUCCAUAAUUCCCGCU | |
| | CUUUCUCUAUAGGAAAUAAGACUUUCACUUAGGGCAAUCUUAACAGACUU | |
| | GAGGUUCAGUAUUUGCUUCUGAAGCCAGGAGACAGAAUCCAUGAGUUUAU | |
| | CAUUUUCUUUCAUCACUUUGUCCACUGAACUUAGGCGCAACCAAUCAGCU | |
| | UCAUUACGUUCCUUGGUUCUGCACAUAUGGUCAAAGGUAUUAUGUAUAGA | |
| | GUCACUAAACUCCUUGCCUCUCAUGAGUGGUGAAUUGGGAGUGUCAAAUG | |
| | CAUUUAUUUUGUAUAACUCUCUAAACAAUCCAUGCCAAGGACUAUCAGUG | |
| | UUCUCCAUACUAUUAGAAGUAGAAUCCUUAGCAUUUUUGAGUCUAAUCAU | |
| | AUUAAGCAGCCAACUCCAGAAACUCCCAAACUAAUGAAAGAACUCCAUUC | |
| | UUAAAAUACUGUUCCUCUAGAACCAUUCUUGGUACCAAAAUCUAUAUUUG | |
| | CCAGAGUUCUCUAGAGGGACAGAACUACUAGGAUACAAAGUCCCACAAUA | |
| | AGCUGUCUUUAAGCUUGAGAAGCAAGGAGAGUGAGUCUGAGUCUCAAAAC | |
| | UGAAGAACUUGGAGUCCAAUGUUUGAGGGCAGGAAGCAUCCAGCAUGGGA | |
| | GAAAGAUAUAGGCUGGGAGACUAGGCCAGUCUCUCCUUUUCACGUUUUUC | |
| | UGCCUGCCUUAUAUUUGCUGGCAGUUCAUUAGAUUGUGCCCACCAGAUUA | |
| | AGGGUGAAUCCUCUUUCCCGAGCCCACUGACUCAAAUGUUAAUCUCCUUU | |
| | GGCAACACCCUCACAGACAUGCCCAGGAUCAAUACUUUGCAUCCUUCAGU | |
| | UCAAUCAAGUUGACACUCAGUAUUAACCAUCACAGAGGGCCACUGUUCUC | |
| | CAGACUCCAGAAUGAUAGAUCCACUGGCAGCUUGUACCCUGCACCUGGAA | |
| | AAGCUACAGGCAUUCAAUGCCAGCCCUUGAGAGCAGCAUUGGGGGGUCGA | |
| | GCCCUGGAAGGCCACAGGGGCGGAGCUGCCCAAGGCCUUGGGAGCCCAGC | |
| | CCUUACAUCAGUGUGCCCUGGAUGUGGGACAUGGAGUCAAAAGAGAUUAU | |
| | UGUGGAGCUAUACAAUUUAAUGACCACCCUGCUGGGUUUUGGACUUGCAU | |
| | AGGGCCUGUAGCCCUGUUCUUUUGGCUGAUUUCUCUCUUUUGGAAUGGAA | |
| | GUAUUUACCCAAUGACUAUACCCCCAUUGUAUCUUGGAGGUAACUAACUU | |
| | GUUUUUUAUUUUACAGGCUCAUAGGCAGAAGGGACUAGCCUUGUCUCAGA | |
| | UGAGACUUUGGACUGUGGAGUUUUGAGUUAAUGCUGGAAUGAAUUAAGAC | |
| | UUUGGGAAACUGUUGGGAAGGCAUGAUUGUAUUUUGCAGUGUGAGAAGGA | |
| | CAUGAGAUUUGGGAGGGCCCAGGGGCAGAAUGAUGUGGUUUGAAUCUGUG | |
| | UCCUCACUCAAAUCUCAUGUUCAAUUGUAAUCCCCAAUGUUGGAAGUGGG | |
| | ACCUGGUGGGAGGUGAUUGGAUCAUGGGGGCACUUUCACAUGAAUGGCUU | |
| | AGCACCAUCCCCUCAGUGCUGUCCUCAUGAUAAUGAGUGAGUGAGUUAUU | |
| | GUGAGAUAUGGUUAUUUAGAAAUGUGUAGCACCUCUCCCCUCUCUCUCUU | |
| | CUUCCUGCUCCACCCAUGUAAGAUCUAACUGCCUCCUCUUUUCCUUCCAC | |
| | CAUGAUUGUAAGUUUCCUGAGACUUCCCUAGAAGCAGAAGCAGCUAUGCU | |
| | UCCUGUACAGCCUGCAGAACAAUGAGCGAAUUAAACCUCUUUUCUUUAUA | |
| | AAUUACCCAGUCUUGGGUAUUCCUUUAUAGCAAUACGAGAAUGGCCUAAU | |
| | ACAAAAAGGUAUACAGUUUGGGAGAGAGGAAAUAAAAUUAUCUUUGCAGA | |
| | UGACAUGACUGUCUUUACAAAAUAUCCUACAGACUGGCAGAAACAAACAA | |
| | ACAAACAAGAAACACCUCUCCUUGAAGGAAAUAAUAAUCAAUAUAACAAG | |
| | GUUUCAGUAUACAGAGUUAACAUACAAAAUCAGUUGCUUUCCUAUACACU | |
| | GGCAAUAGACAAUUGAAAUUUGAAAUUAUAAUAAUAAAAUGUCAUUUACA | |
| | GAAGUGAUACCCUCCUUCCCCCAACGAACCCACCAAUUCAACAACAACUC | |
| | ACAGACAAAUUCUCUUACUGAGAAAUCCAAAACUAGUUGAGAGCCUCCUG | |
| | CACCCAGACACAUCAAAUCCCACAGGAAAAUUCAGGACACCCUCAUGUCU | |
| | GAAUCCCUACCCACAGCUCAAUGCCACAUGAUUGAAAGGAAUCAUAGCUC | |
| | CCAGCUUCUCCCUGGAGAGUAAAAGAGUUGGACCAUGUGUAUGACACUCU | |
| | UCCAACUUCUUCCUCCCCAAAGGAUGGGCCUUUCUCUUUCCAGUCUUGGA | |
| | GCAGUAAUGGAACCCAGCAUAAUCUAGCCAUCCAGGGGCUGGUAAGAACA | |
| | AAGACAGAAGUUUGCACUGACAGUUACCAUAUCCUUUCCCUCAGCUCAAC | |
| | ACAGAGCAAGAAAAUGAAAAAUCCCUGCUUCCAACAUCUUCUUGGGAAGG | |
| | GAAGAGUUAGUCUGCUUGGCCAAUGCCCCAGUGUUUCUGGGAGCUUCCCA | |
| | AAGGACUUCCUCUGUCUGGCUUGUUUUGGGGUACUUAUGGGACCUGGCAU | |
| | ACUCUAGCUGCCUUGGGGCCAAUGAGAACAAAUAUGGUGGUUUGAUCUAG | |
| | CAUGCAACUAGAUGAUGCAGUCCCACCCACAAGCUCAGUGUAGGGUGAAC | |
| | AGGUGAAAAACCACAGAUCCCAGCUUCUCUCUCUGUGUGUCAUGGGAAGA | |
| | GUUGGAAAGGGCAUCAAAUGAUUCAACUUUUCUGGGGGCUACUUGAGGAA | |
| | UGGCCCCAAUAUCACUUCUAUUGGAACACUGAUUGUACCUAGCAUACUCU | |
| | AGAUGGUAGCUGCUAAGAACAAAGAAAACAGAUUGGCAGGUUGGAGAGGC | |
| | CCCGAGAAUCCAUGGCUGAGCUGGUUAGUGAGGGUCUUCUCCUUUAUAAG | |
| | UUUAGUCUGUGAAGAUUGGGAGAGGUCACUGUUUUGUCUAAUAUACCAAC | |
| | CCUACACAAAGAGUCAAGGAAAAUAAAAAUAACAUGGAAAUGGAUUCCAA | |
| | ACAAAGGAACAAGAUAAAUCUCCAGAAACAAAGCCUAAUGAAAGAGAAAU | |
| | AUUUGAUUUACCUAACAGAGAACUCAAAAUAACCAUUAUGAAGAUGUUCA | |
| | CCAAAACCAAGAGAACAAUGCAUGAACAUGAGUAUUUCAACAGAGAUAUA | |
| | GAAAGUAUAAAAAGUACCAAAAAGAAUUUGUAGAGCUAAAUAAUACAAUA | |
| | ACAGCUCAAAAAUUCACUAAAGCAGUGUGACAGCAGACUAAAUCAAGCAG | |
| | AAGAAAAGAUAAGCACACUUGAAGACAAGUUAUUGUUGUUAGAAAUUAUU | |
| | CAGUCAGAAGAGCAAAAAGAAAAACGAAUGACAAAAAUAAAGACAGCUUA | |
| | AAGGACUUAUGGGACACCAUCAAGUGGACCAAUGUGCACAUUAUAGGAGU | |
| | UCUUGGAGGAGAGAGGAGAGAGAGAAAGAACCACAAAACAUAUUCAAAGA | |
| | AAUAAUAGCACAAAACUUCCCAAAUCUGGGGAAGAAAAUGCACAUUCUAC | |
| | UCUAGGAAGCCCAGCAGAUACCAGAUGAAAUGAACGGAAAGAAACCUACA | |
| | CUGAGAUACAUUAUAAUUAUAUUGUCAAAAGUCAAGGCAAAGGGAGAAUU | |
| | UUCAAAGUAGCAACAGAAAAGUGAUUUGUCAUGUACAAGGGAUUUCCCAU | |
| | AAAACUAAAAGUGGAUUUUUCAGUAGAAAACCUUGCAGGCCAGAAGGGAG | |
| | UGGGAUGAUAUAUUCAAAGUGCUGAAAUUAAAAAAAAAAAACCUGCCAAU | |
| | GAAAAAUACUUGGCAUGCCAAAAUUCUCCUUCAAAAAUAGAGACUUUCCC | |
| | AGACAAAUAAAAGCAGAGAGGCUGGGCGCUGUGGCUCAUGCCUGUAAUCC | |
| | CAGCAUUUUGGGAGGCCACGGCGGGCGGAUCACUUGAGGUCAGGAGUUAA | |
| | AGACCAGCCUGGCCAAUAUAGUGAAACCCCAUCUCUACUAAAGAUACAAA | |
| | AAAUUAGCUGGGCAUGGUGGCAGGCGCCUAUAAUCCCAGCUACUUGGGAG | |
| | GCUGAGGCAGAGAUUGCAUUGAGCCGAGAUCAUCGUGCCAUUGCAUUCCA | |
| | GCCCUGGCAACAGGGCGAGAAUCCGUCUAAAAAAAAAAAAAGUGGAGAGU | |
| | UGAUCAGCGCCAUACUUGUCUUAAAAGGAAUGCUAAUGGAAGCUCUUCAA | |
| | GUUGCAACAAAUGACACAAACGGUAACAUGAUAGCACAAGAAAGUGAAAC | |
| | UUGGUAAUGAUAAAUAUAUAGGCAAAUUCAGACUACUGCAAACACUGUAA | |
| | CUGUUGGAUGAAUCACUUUUAAUUAUAAAGUUAAAAGACAAAAGAUAUUG | |
| | AAAAUAUCUGUAAUUAAAUAUUAUUUAAAGGGAUAUGGAAUAUAGAGAGA | |
| | UGUGAAUUUUAACAUCUAUAACAUAGUGUGUGAUGGGAAAGAAGUGCAAG | |
| | UGUAGAGAAAUUGAAUGUAAGUUGUUAUCAGUUUAAUAUAGACUAUUUUA | |
| | AAUAAAUUAUAAGAUAGACUAAUAGAUAUUUUAUGUAAGCCCCAUAGUGG | |
| | CCACAAAGAAAUACCUAUAGAAGAUACAAAAAAGGAAAAGAGAAAGAAAU | |
| | CAAAGUAUAUCAGUACAAAAUAUUCACUGCAACACAAAGGAAGACAGCAU | |
| | GAGAGAGUAAGACAUAGGAGGUCAAGGUAGGAGGAUUACUUGAGCCCAGG | |
| | AGCUUGAGAUCAGCCUAGACAACAUAGCAAGACCCUAUCUGUAAAACAAU | |
| | AUAUUUUUAAAAUUAGUUGAGCAUGGUGGCAUAUACCUGUAGCCCUAGCU | |
| | GCUCAGGAGGCUAAUGCAGGAGGAUUGCUUAAGCCUAGAUAACAAAGAGC | |
| | UAUGAUUGUGCCAGUGCACUCCAGUCUGGGUGAUCCUGUCUCAAUAAAAA | |
| | AUAAAAGACAGAGAAAGAAUAAAAAGACAGCUGUCAGAACCACAAGACAA | |
| | ACAGACAACAAUGAACAAAAUGGCAAUAGUAAAUUUUUCUCUAUCAAUGC | |
| | UUACUUUACAUAGACAUGGAUUAAAUUCCUCAACCAAAAGACAAAGAGCA | |
| | GUUGAAUGAAUUAGAAAAAUCAGAGUAAACUAUGUGCUGUCUAUAAGAAA | |
| | CUCACUUUAGAUUUAAAGACACAGAUAGUGAAGGGAUGGAAAUAUAUAUU | |
| | CUAUGUAAAUGGUCACCAAAGGAGAAGUGGCUAUACUUAUAUCAGACAAA | |
| | AUAUAUGUAAAGUAAAAAUUAGCACAAGAUACAAGGAAGAACAUUAUAUA | |
| | AUAAUGAAAAGGUCAAUUGAACAGGAAGAUAUAACACUUAUAUGUACCCA | |
| | ACAUCAGAGCAUCUAAAUAUAUAAAGCAAAAAUCGACACACCUAAAGAAA | |
| | UAAUACACAAAAAAUACAAUAAUUAUAGGAGGCUUCUUUACCCCAUUUUU | |
| | AAUAGUGGAUAAAACAACCAGACAUAAAAUAACAAACAGCAGACUUGAAC | |
| | AGCACUAUAGACCAAAUGAGCCUAACAUACAUAUAUAAAAUAUUCAUCUC | |
| | AUAGCUGCAGAAUAUACAUUCUUCUCAGGUGUACAUGAAACAUUCUCUAG | |
| | GAUACAUCACAUGUCAGGUCACAAAACAAUUCUUAGUAAAAUUAAUAGGA | |
| | UUGAAAUUAUACCAAAUAUCUUUUUGGAGCACAAUGGAAUGAAACUAGAA | |
| | AUCAGUAGCAGAAGGAAAACUGGAAAAUUCACAAAUAUGUGGAAAUUAAC | |
| | ACACUCCCGAUAUCCUAGUAUUCUUCGCUUAAUGGGUCAAAGAAAAGAAU | |
| | AAGGAAAGAAAUUAGAAAAUAUUGAGGCAAAUUAAAAUAAAAAUACAACA | |
| | UACCAAAACUUAUGGGAUGCAGAAAAAAAAAAAAAGAAUGUGAUAUGCUG | |
| | GUAUAGGACAUUUACCAUGAAUGGAAUUAACAGGAUUGAUUAUAGCUCUG | |
| | GGUGAGUCAGUGAGUGAAUGUGAAGGCCUAGGAUGUUACUAUACACUACU | |
| | AUAGAUUCUAUAAGCACUGUACACUUAGGCUACACUAAAUUUACAAACAA | |
| | AUAUUUUUCUUCAAUAAAAAAUUAAUCUGAGCUUACUGUAACAUUUCGUU | |
| | UUAUAAAAUUCUUAAUUAAAAAAACUUCUUAACUUUUUAUGAUAAUACUU | |
| | AGUUUAAAACACAAACACAGCCGGGUGUGGUGGCUCAUGCCUGUAAUCCC | |
| | AACACUUUGGCAAGCUGAGGUGGGUGGAUCACCUGAGGUCGAGAGUUCGA | |
| | GACCAGCCCGACCAAUAUGGAGAAACCCCAUCUCUAAUAAAAGUACAAAA | |
| | UUAGCCAUGGUGGCAUAUGCCUGUAAUCCCAGCUACUCAGGAGGCUGAGG | |
| | CAGGAGAAUCACUUGAACCUGGGAGGCGGAGGUUGCGGUGAGCCGAGAUC | |
| | ACGCUGUUAUACUCCAGCCUGGGCAACAAAAGCGAAAUUCUGUCUCAAAA | |
| | AAAACAAAACAACAACAACAACAACAACAACAAAAACACGUUGUAUAGCU | |
| | GCAUAAAGAUAUUUGUUAUAUCCUUAUUCUAUAAGCUUUUUUCUUUUUUU | |
| | UUCUUUUUUACGUUUUAAAGUUUUUUGUUAAAAACGAAGACACAAGCACA | |
| | CACAAUAGCCUAGGUCCUUGCAGGAUCAGGAUCAUCAACAUCUGUGUCUU | |
| | CCACCUCCACAUCUUGCCCCACUGGAGGGUCUUCAGGAGCAGUAACACAC | |
| | AUGGAGUUGCCAUCUCCUAGGAUAACAAUGCCUUCUUCUGGAAUACCUCC | |
| | UGAAGGACCUGUCUGAGGCUGUCUUACAGUUAACUUUUUUUUAAUAUACA | |
| | UAAGGGUAUAUUAUAAAAUAAUGAUAAAAAGUCAGCACAGUAGGUUUGUU | |
| | UACACCAGCAUCACCACCACAAGCACGUGAGUAAUGCUACGACAUUAUUA | |
| | UAUCACUAGGCAACAACAAUUUUUCAGCAUCAUUAUAGUCUUAUGGGACC | |
| | ACCAUCAUACAUGUGGUCCAUCUUUGACCCAAAUAUUAUUAUGCUAUACA | |
| | UGACUGUAUUGGAAAUAACAAGGAAAGAAAUAGGAGAACCUCAGUUCCCA | |
| | AGGUGGAGAUCAGUUGUUGUUAAAUAUUAGAGUGCGUCAGAAUCACCUGA | |
| | AGGACUUAUUGAAACACAGAUUGCUGGACCCUUAGAGUUGAGUGUCUAGG | |
| | GUAGGACCUAACAUUUUGUGUGUCUAACAAGUCUCCAGGUGAUAGUUAUG | |
| | GUAUUGGCCUAGGGACCACAUUUUGGGAACUAUCGGCCUAGAUUUUCUCC | |
| | AAAAGUAGGAGGUUAGGUCAACUGCUGAGAAUAAGGGAAAGUGGGCCCUU | |
| | GAACUCUCCUUUCAGCCAAAGGCAGGCAUGUUCUAAACACUGAAUCAUGU | |
| | UAAAAGUGAAUUUUAAUCUGGGGGCAAUUCUAUGCCAAGCCAACUGAUUA | |
| | GUGGUGGGAUUAAUUCCUAGGUUUUAGAUAACAGCACUGCUUUGCAAAUG | |
| | CUCACAGCUUAAUGAUACCAUAGAAUUAUUAUCAGCCUCAUUUUCACUUG | |
| | UGUAGGAAGAGGCACGGAAAUAAAGAAGGCAGGAGUCUUCUUUCAGGUUU | |
| | CCUUAUGGUGGACUCUUUCAUAAGAAACAGUUCUGGCUUCUCCUUUGUUG | |
| | UUCCCUCAAAGAAGAUUAUAAAUAUGGUUGCUUGUCUCCCACCUCCAUCU | |
| | GCCUGAGGGCUGUGCUUUGCUUCUGUAAAUGUUUCAGUUGGAACAUGUUG | |
| | AUUCUGUUUGAGCAUCUAUUAACUUAGGUGUGAUGUCUAAUGUUCACUGU | |
| | CUCAACGUUCACCUCCACUGGGGAGAGUGGUGGCGAUUUCCCUGCCUCUG | |
| | UCUAAGGCUGGAAACAUUCGUUAUUGUGGAGGGGAAAGUGGUUGGUUCAC | |
| | UGAGGUGGGGGCUCUGAAACACAGGAUGUGCUUUAUUGCUCCCACAGUCA | |
| | GUCACAAGUUGACAGUCGCAUGAGGCUUUCAAUAAAAAUAUUUUAUACUU | |
| | UUGUGCUGCCUCUCAGCUGCAGAUCCUAAAGCCCUGUUUUAAAGAUCAAA | |
| | AUACCCAUAGAUGUGCAAAUGCCAGGGAUUCAACAAAAGCUGGUGCAUGG | |
| | AUGGGAAACUGAUGACACACACAUCUGUUCAGCUCUCAUCAGGCUUCACC | |
| | ACCCACUGGAAUCUUUUGGCUACAGAAACUGAAUUUGUGAUUCGUGCUUC | |
| | AAGUACUGCAGUCAACAACUCAGCAAUUCACAAAGUCUCUUCUGUGAUUG | |
| | CCGUCUUAGGUAUGUGAGGUAAUGGACAAUGGAUAUUUAAAAUUGUGCCU | |
| | CAACCCUGCUUUACCUCAUCACUUAUUCCGGUAUCAGCCAAAUUUAUAUU | |
| | AGAAUAUGGGAAGUGCAGGUUUUAUAUAAAAGGUGCCUAGGAUAGAUUCU | |
| | GGCCUAUAUAGCUCUAAUAAUUGGUAUAAUACUUUACAAUAUAGAUGAUA | |
| | UUCUCACUGUAACACAUUAUAGUAUACAACGUCCAUUCUGUCCCCUACUU | |
| | UAUUUAAACUCAACUCCAUAAGAACCUUGUGAGAUGGGCAUUAUUCCUUA | |
| | AGGUAAAAGAAGAACAAACAAAAAAUGGCUGCAACCAAAAUGUAAUAAUU | |
| | GGUGUGAAUAGUUUUAUAAUAAAAAAGAAAGACUUCACUUUGGAUAUUUG | |
| | AUGGCUGACACCUUUCAGUUUCCCACCUAUCUUCCUUUUUCUCCUUAGCC | |
| | CCACAUCCUGAGAAGAUAAGAGAACCUGGGCUCUCCUCCACUGGUGCCCA | |
| | UGGGGAAGUUCAAACCACACAAAUCCCUGUCCUCUAGGAAUUCUCAUUCU | |
| | AGUCCCAACCCUCUUAACUAUGAUAAAAACUCUAAACCAUCAACACCACC | |
| | CCCAUCUUGACUUGUGUCACCUUAGGUAGAAUCUGAGCCCUUCACCUUGG | |
| | GGAAUAUGUGAGUAGUAAACUUUAUUUUAUAUCUGUAAUUCCUAUGUUUU | |
| | AUUCAUCCUAGCAUUCAUAAAUUUCUGAGGAUGCUGGAUGUUCAUAUAAA | |
| | UAGGUUUAUUAGCUUUAGAUAGACUAAUGAUGCUUUAUUCAAAUGUGUGG | |
| | UUAUUUCAGAUCAAAGAAAAGGAAGCAUUGUGCUUGACACAGGGCUAAGA | |
| | ACUGAAAAAUGAACAAAAUAUGGCCUGUGCCCUUUCCUUAGUAGUUAUGG | |
| | UCUGGUAGAAAAGGCAGACAUGUACAUUAAAAAAUUAUAAAACAGUGUCA | |
| | UAUGUAGCAUGUACAAGUUAUUGUGGGAAUGCAGAAGAGAGAGUGGCUGG | |
| | UUUUUCCUUGGUUUAGCAAUAUGUGUGUAAAGUCAUAAGAGCAUGAAAGA | |
| | AGAUUGUAUGUUGAGGAAACUAUUUUUAGUUUUAUUUGGUUGGAGAGCUG | |
| | UAUUAGUGUGGAGAGAUGCAAUUUGAAAGGGCCUUAAAGGCUCUGCUGGU | |
| | ACAUUGGAUUUUAUCCUGAGGGCAUUGGUGAGUUUUGAAGGGCUUUAAGA | |
| | AAUGAAAUAAAAGAAGAACACUUUUGAAGAGUUCACUUUGAUAGCAGUAU | |
| | GGAAGAUAGAUGGGAGCCGAGACAGAAAGAUACUUGAGAACAAUACUACU | |
| | UUAGUCCAAGUGAGCAAUGACAAUGGCUAAAUCAGGACAAAAGCAGUGAG | |
| | AAUGGUUAGAGGAGGACAUGUGCCAGACACUGUUCCAGGUGUUGGGAUAU | |
| | AAGGUUGAAAGAGGACAGAGAAAUUCUGUUUUUUAUGAUGUUUUUAUUCU | |
| | AAUAAGGGUAUAGAAGAGAACCAUUAAGAUCCAUUCUCUUCUCUCCAGCA | |
| | GUAAGAAAACUCCACAGAUGCAGCUGAACACAUGACUACCCAGAAAAUAG | |
| | ACUACUUUUCAGCCUUUGUUGCAGCUUCCUCCAAGUCUAGCUAUACUAGG | |
| | UUCUGGCCAAAUCAUGCCCUUGAAAUGAAGAGGCAUCCUCCUCCUUUCCC | |
| | UUUUCCCUUUCUACUGGUUGGAAUGGGUACACAGUGAUGAGAUUUCUUGC | |
| | ACCAUGUAGCCAAGGUUACCACAGGCCUGAUAGAACAGCAAGACAAAAGG | |
| | GCUCUGAAUAUGUAGCAACUUCAUGGACAGCUGUCAUACCAGGCUGAACU | |
| | UUAAUGUGAAAUAACUAAAAUUCUGUCUUGUUGUUUCUGGGUGUCUCUUC | |
| | AUCACAGAAGCUGAGCCUACCUCAUAAGUAAUACUGGGGGAGAUAUACAG | |
| | UCAUAAUGGAGAUUUACAUGAAGAGAAUGCUCAGGUCUUGAACCCCACAA | |
| | AGGCAGUCAUCUAAGUGUAUAAAUAAGUUAUAUAAGUAUAAGCUGAAGCU | |
| | GAGUCUCUUAAGCUUAAUAUAUAAUUAUUCAAAAUAAGUACUAGUUGUAA | |
| | UUUAUAUGCAAGGAAAAGCUUUGUAGACUGUCUGAGCUAGUCAGUGAGGG | |
| | CUCCUCAGACAAGAAAGAUCCACUUACGCAAGGCACUUACACGUGGGAGU | |
| | GCCAGCCAGUGUGAUGAAAGAGAGACUAGAGCUGAAUAUCUCAUCCUAUG | |
| | UCUCAUUAUUUGAGGAACUUACUAAUCCUGUAUUAAGAAUUUGGUUAAUU | |
| | AUUUGAUAAAGGCAUGAGUUUGUUCAUUGAGCUGCAAGCUGAUUACAGUA | |
| | GUAAUUACAAUAACUAACAAGAAAACUAUAGAAUGAGAUAAAUAUAAUAA | |
| | AUGAAAUAAACAAGGUGAUGUGAUAGAGUAAAUAUAAGCUGAGCAAAAGA | |
| | AUGCUGCUUUAGACAAGUUUGCUACGGAAGUCCUCCAAGAGAGGUGAUGU | |
| | UUGAUUGAUACACAAAUAAUAAAUGGCAGUUAUUGUAUAAAAAGUAGGGG | |
| | GCAGGGUAGGAACUUUAUAGGCAGAGGGACUACAAAUACAAAAGCUCUGA | |
| | GGUGAGAAAGAGCCUGGUAUAUCUUCAUAGGAGAAAAAAGGCCGGGGUGA | |
| | AUGGAGAAUGGAAAGCGCGUGGGGAGAAAGAAGGAAGUUGAAAUUGUAGA | |
| | GGGAGGGAGCAAUUAGAUCAUCGAACUGCUUGUAGGCUAUAUUAAGGAAU | |
| | UUUGUUUUAUUCUAACAGCAAGGGGAAGCCAUUGUACAGUUUUAAACAAA | |
| | AACAUGACAUUAUUUGACUUUAAAAAAAUAAUGUUUUGGUUAUGUAUAGA | |
| | AUGAACCAUAGAAGCAAAGUGGAAGCAGAGAUUAUUGCAGGAAGGUAAUU | |
| | UGAAACAGGGUGAUGCCAGAGGGAAUGGGAACAAGUGAAAGGAUUUAAUU | |
| | GCUUUUUAGAAACAUUGAUAGGGCUUACUAUGAGAUGCAGGUAGAGGUCU | |
| | GAGAAAAGAAGUCUUCAAGAAUCAAUCAUGCUUUUUGCUUGAUUUGCUAG | |
| | GUUGAUAGUGGUACCAUUUGCUAAAGCUGAAAACACUCAGGGAAAUAUUU | |
| | UUUGGGGUAUAAACCAAAAGAUCAAUUUUGGACAAGUAUAAAAGUGUUGA | |
| | UGGCUAUAGGAAAUUAGGUGUAUGAAUCUGGGUUUCAGACGAGAGAGUGA | |
| | AAUUUCAGAUGAGAGAGAGAAUAAAUAUAGUAUUAAUACUUUAAUAGCUC | |
| | AUUUUAUUUAUAAAAUAUUUCAAAUAUACUAAAAGUACAGAAAAUAAUUU | |
| | CAUAGGCAUAAUCUCUUGAUGUACCCACCAUAUAGAUUUAACAAAUGUUG | |
| | AUAUUUUGCUUUUUUUUGCUUCAGAACUUUAAAAAAUAUAAUGUUGCAGA | |
| | UAAAGUUAAAGGAUGAGAGCAUUCUUAAGGAUGAUGGGAAGACAGAGGUU | |
| | UGAAUGUUAUCUUACUUCUUGUUCACUCCCAAUGCUACCUCUUCUGCCAG | |
| | CAGACAGGUCCUAGAGGAUAGGAAUGAAGCCUCAGGUUCUGUCACAGUCU | |
| | UGCCAAGGAGUGCAGAGGAAUUUCUCUUCUUUGGGAAGAGGAGAGCUUGG | |
| | GCAUCAGCACUGAAGAGCCCCAAGAUUAGGAUCAGAAUGCCUGGAUCCUG | |
| | GGACAGCUUCGCUCUCAGAAACUCAUAAUCUCCCACAAAUUCACAUCUAA | |
| | AGAAAUAUACCAAAAUUCUUAUUUCCGGAUUCCUAAAAACUGAGAAGUGA | |
| | CCAAACAGGCAGCUGGACCUUUUCUAUCUCUUGGGUUGGGAGAGCAUGAC | |
| | GAGGCCAGAGCAAGAAGAUCAGAAAGGUUUCCUUCUGAUGCCUUGACGUG | |
| | GCACCAGCAAGGUGUCCUUUACCAGAGGAAAAACCAGAAAGGAGAAAACC | |
| | UUUAAGAUAUACCUCAUGAAAUAGAGUUACUGGGGGAGGCAGACAGUGAC | |
| | CCAUGACAAAAAACAAAGUCCUUGGGAGAUUCAAAUACAAAACAGAAAAA | |
| | AACUUUCUGGAACCAAAAUAAUUUUAAGAAUUUGACACAAAAAUAUGUAU | |
| | ACAUUGUGAAAUAGGUAAAUGACAACAUGUGCAUUACCUCAUAUACUUAU | |
| | UUUUUUGUGAUGAAACACUUAAAAUCUAUUCUCUUAGCAGUUUGCAAAAA | |
| | UAUAGUAUGUUGGUGUUAACUGUUGUCAUCAUGCACAAUAGAUCUCUUGA | |
| | ACUUAUUCCUCCUAGGUGAAGUUUUGUAUUCUUUGACCGACAUUGCCUCA | |
| | AUCCACAAUCUCUCCAUUUUCUCUGGCAUCAGCCUUUUCAAAUCACCUUC | |
| | CUGCGAUAGUCUCUGCUUCCACUUUGUCCCUACAAUUCAUUCUACCCAUA | |
| | CCCAAAACACUAUUUUUCAAAGUCAAAUUACAUCAUAUUUUUGUCCAAAG | |
| | CUGUGUAAUGGCUUCUUAUUGCUGUUAAGAUAAAACAAAAUUCCGUUCCA | |
| | UAGCCUAUUAGCAAUUAGGUGGUCUAACUACUCCCUGCCUCUACCAUCUC | |
| | AACUUCCUUCUUUCUCUUCCCUUGCUUUUCAUUCUCCAGACACACCGACC | |
| | UCUUCAGUCCCUGCUAAGCAUAAUUCUACUCUCCACUUCUGAGUUCAAUU | |
| | UUUUUACACUCCAUAUAUAAGUGAGAUCAUGUGGCAUUUGUCUUUCUAUG | |
| | CCUGGCUUAUUUCACGUAACAUAAUGUCCUCCAUGUCCAUCCAUGUUGUC | |
| | ACAAAUGACAGAAAUUCCUUCUUUUUUUUUUAAAGAAUAAAGAAUAUUUC | |
| | UUUUCAUAUAUAUGCCACAUUUUCUUUCUCUGUUCAUCCAUUGAUAGACA | |
| | CUUAGGUUGAUUCCUUAUCUUGGCUAUUGUGAACAGUGCUGCUAUUAACA | |
| | UGGUAGUGCAGAUAUUCAACAUAGUUAUUUCAUUUCCUUUAGAUGUAUAC | |
| | CCAGUAGUGAGAUUUCUGGGUCAUAUGGUAGCUCUAUUUUUAAGUUUUUG | |
| | AGAAAACUUUCUACUGUUUUCCACAAUGGCUGAACUAAUUUACAUUUUCA | |
| | UCUACACUGUAAAAGGGUUUCUUUUUCUCCACAUCCUCUUCAACACUUAU | |
| | CUUUCACUUAUUUGAUAAUAGCCAUUCUAACAGGUGUGAGGUAAUAUCUC | |
| | AUAGUGGUUUUAAUUUGCAUUUCUCUGAUGAUUAGUGAUGUUGAACGUUU | |
| | UUUCAUAUACCUAUUGGGCAUUUGUGUUUCUUCUUUUGAGAAAUGUCUAU | |
| | UCAGAUCUUUUGUCCAUGUUUAAAAUUAGUAUUGAGUUGUUUGAGUUCCU | |
| | UAUAUAUUUUGGCUAUUAAUCCCUUAUCAGCUGUGUGGCUUGCACAUAUU | |
| | UUCUCCCAUUUUGUAGGUUGUCUCUUUACUCUGUUGAUUGUUUGCUUGGC | |
| | UGUACAGAAGCUUUUCAGUUUGAUAUAAUCCCAAUUGUUUAUUUUUGUUU | |
| | UUGUUGCUCAUGCUUUCAGGUUUAUACCUAAAAAAAGUAUUACCCAGACC | |
| | AGUGUAAUGGAGCUUUUCCCCUAUGUUUUCUUCUGGUAGUUUUUCAUUUU | |
| | CAGAUCUUACAUUUAACUCUUAAAUCAAUUUUUAGUUGAUUUUUGUUUAU | |
| | GGUAUGAGAUGAGGGUCUAAUUUCAUUCUUUUGAAUGUGAAUAUCUAGUU | |
| | UUGCCAACAUCGUUUAUUAACGAAUAUUCUCAUUGUGUAUUCUUAGCUCC | |
| | UUUGUCAAAAAUCAAUUGACCAUUAAUGUGUGGAUUUAUUUCUGGGCUCU | |
| | CUGUUCUUUUGGUCUAUGUAUCUGUUUUUAUGACAGCACCAUGCUGUUUU | |
| | GAUUAUUAUUGCUUUGUGGUAUAUUUUGAAAUCAGGUAGUGUGGUGCCUU | |
| | CAGCUUUGUUAUUUUUGCUUAAGAUUGCUUUAGCUAUUCAGGGAAUUCUA | |
| | UGGUUCCAAACAAAUUUUAGAAUUGGUUAUUUUAUUUCUGUGAAAAAAUG | |
| | CCAUUGGAAUUUUGAUAGAAAUUACACUGAAUUUGUAGAUCACUUUGGAU | |
| | AGUAUGGACAUUUUAACAAUAUGAAUUCUUCAAAGCCAUGAACAUAGAUU | |
| | CCCUCUCCAUAUAUCCGAGUCUUCUUCAAUUUCAAUGUUUUACAGUUUUC | |
| | AAUGUGCAGGUCUUUCACCUCCUUGGUUAAAUUUAUUUCUAAGUAAUAUU | |
| | UUUGUAGGUAUUGUAAAUGGGAUUGUUUUCUUGAUUUCUUUUGUGGAUAA | |
| | UUUGUUGUUAGUAUAUAGAAAUGCUACUGAUUUAUAUGUUGAUUUUAUAU | |
| | ACUGAAACUUUACCGAAUUUUGUUUAAUAGUUCUAAUAAUUUUUUGUGGA | |
| | GUCUUUAGGGAUUUUUAUUUAUAAGAUCAUGUCAUUUUUACAUAGGAACA | |
| | AUUUAACAUCUUCUUUUCCAAUUUGGAUGUCUUUUAUUUCUUUCUCUUGC | |
| | CUAAUUGCUCUGGCUAGGAUGUCCAGCAGCAUGUUGAAUAGAAGUGGUGA | |
| | GAGUGGGCAUCAUUGUCGUGAUUCAUAUCUUAGAGGGAAAGCUUUUGACU | |
| | UUUCACCUUUGGAUAUGAUGUUAGCUAUGGGUUUGUCAUAUAUGGUCUUU | |
| | ACAGUGUGGGGUACAUUACUUCUAUACCUAAUUUGUUUAGUUGAUUUUAU | |
| | UAUGGAAGAUUUUUGAAUUUUGUCAGUUGCUUUUUCUGUAUCGAGAGGAU | |
| | CAUAUGGUUUUUGAUUUUUUUUUUAGAUUUGCAUAUGUUGAAUCAUCCUU | |
| | GCAUCCCUGGGAUAAAUCCAACUUGAUUAUGGUGAAUGGCCCUUUUAAUG | |
| | UACUGUUGAAUUCAGUUUGCUAGUAUUUUGUGAUGAUUUUUGCAUCUGUG | |
| | UUCAUCAGGGAUAUUGGCCUGUAAUUUUCUCUUCCUGUAGUGCCCUGUCU | |
| | GGCUUUGGUAUCAGUUUAAUGCUGGUCUGAAAAUAUUUAUGUAGCUCAAG | |
| | AGCAAGAUUAGAUAUAGAAAUGUGGACUUAUAAGUUUUGGGGCAGAUAAA | |
| | UUGAUAGUUGAAUGUUUUAUAGACAGGGAAAUGGAAUCCACAAAUGAGUU | |
| | UCUAGAAUGAGAAAAGGACAACAUUUGAAAGGAGGGGGAAUGCAAAGAGC | |
| | AAGUUAAGACAAUGGAGAUGACCAGCCCUGACCAUACAAGAACAAGAUCA | |
| | GAGACAGGAAAAAGAAGCAAUUAAGCAUACUGCAGGUAAGUCAUGUAAAA | |
| | UAAAGGCAGGAAGUGUAGCUUGGGUCUGGAGAUUUGGAGAGCAUUUAUGA | |
| | CUUCCCAUUUCCCCAUAGGCCCUAUGAAUCUAGUAGUCCUGGUUCUUACA | |
| | UGGGGAGCAUUCCUACCAGAGAACAUGAUAAAUAUACCACUCAAAGCUAU | |
| | AGCUGCUACCUGGUCACUUUGGGCUCCUUAUACCAGUAGAUGAAUAAGCA | |
| | AAGAAAGAAGUUAACAUAUUCUCUGGGAUAAUGUAUCCUGAUCAUCAAGG | |
| | AAAGUUAAGGCUGCUGCUAAAUCAUGGAAAUAGGCAGCACAUAUAUUGCA | |
| | AGGAGAGUAAAGUUGAGCAAAGGCACUCCUACUUCUGUGCCUUGACUCCC | |
| | UGCCCAUGUGUUCUCCUAUUUGGACUGUAGAACCACAUAAUGGCUAUUGG | |
| | UUUAAAGUGUAUUCCUCAUCCUAAAGUAUAGGGUUCCAUGCUAUAGAGUG | |
| | UCAUUUUCCCACUAGCACCUCAGUGGCAAAUUUGAGUCCAUUACAUAUUU | |
| | UUCUAUUGCUGGCAGCUUCUGGAUGGUGUGGUGGUCAUAGGAUUAGUGCA | |
| | CUCCCUGGCUAUUUCCUCUACAUCACACAUCCUUUGCUGUAAAGUGAUCU | |
| | CUUGGCCUGAGUAAAUAUUUUGUAGAUUCCAAUUUGGUUGUCAUAUGCUU | |
| | UGAGUUUUUGGAUAGUGAACUGGCUGAGACACUGUAGGCAGGAAAGGCAA | |
| | ACCCAGACCAAGAAUACACGUUAAUUCCGGCUACAGUAAACAUAACCUUU | |
| | CCCUGGGUGAAAGGGUCCUUAUGGAAUCAACUUGCCACCGAAUGGCAGGU | |
| | UGGUCUCCUUGAGAGAUGGUCUCAACUUUGCUUUCUGUUGCAAGGAGGUC | |
| | CAACAUUCAUCAACAGCAGUUACUAAAUCAGCUUUGAAGAGAUGGAGCCU | |
| | AUGCCGUUAGGUCCAUUCAUAGACUCUGUCUCUAUCACUGUGGCUACACU | |
| | GUAUGUAAGACCACUUUCUAGGCAUUAAGGUGGUCAAGUAUAGAGAUUUG | |
| | CUGACAUAUCUGGCCCAGUCAUCCUUGGGGUUCUGUAGGAUUCUUCUGCA | |
| | GUGGAUUCUGUCUGGUAGGCACAAAUGUACAAUAGAACAAUCCCUGCACU | |
| | UUGUGCCCACUCUCAUAGGUUCAUCCAUAUGCCUCUUACCCAGACCUUUU | |
| | CAUACCUGGUCUUCCAAUCUUGUUUAUUUCAAGGCUGUAACCAGUCAGCU | |
| | AGGUAAGUCAUUAGUCCUUAUAUCCCAGUUUUAUGCCACUUUUCUUCUUU | |
| | CCCUAACUACUGUUUUUCAGUGCUACUCUGUGGUACAACAGCAAUUCAUU | |
| | UUCAUCUUACACCCACACACUGAGUUGAUCCAUCCAUGUACUAAUCUUGG | |
| | GCUUUUUCUCCCUCAUCAACAGGUCACAGGAAACCCUUCAUGAAUUCAUA | |
| | GGUGUGAAAUAGGGGAGAAGCAUUAUAACAGUGACAGAAAGCAUGCGGGU | |
| | UUGUGACAUAUGUUUGUGUAUCUUUCUUAUACCUUCUUACACCUGCUCAA | |
| | GUUUGACCCUGGGCACAUCCUAUCCAUGUUAUGGUUGAUUGCUGCUGGGG | |
| | UCACCUGAUUUUAUGAUUUGUUGGAUCUAUUGGUAUCUGUGGUGUGCUAG | |
| | UAAAUGAUUACUUCUGAAAAAUACUGCCUUGGCUUGUAGCAUUUGGUGGC | |
| | UUCUAUGGCAUAAAUAUGUCUACUAUGGCUGAUUUCAAGUGCCAAACUGA | |
| | UGUCAGAAUGUGGAGUUAGGAAGAGAUGUGAUGGUAUGAACUGUCUCUAG | |
| | CACAUCACUAUCUUCAGCUGGUGAGAGCAGUUCUGGUCACGUGGUCACUU | |
| | GAUGUCAUUUGAGUUUCCAUGGUUUGUUACUCAUUUUCUACUGGGGCCCA | |
| | AUGGUAUGCCAGGAAUUGUUUAUCAAUAGUUCAUAGUCCUGUACUGUAGA | |
| | UGUCAUAGCCUUGCCCUGGAACCCUAGAAGUGUGCACUGUAUAAAACACA | |
| | UUUUGCUACUACAAUUAAUUCCAGUACUAUAGGUGAUGUGGUGCAAGCAA | |
| | GAGUCCUGGACCUUAUGCAGACCCAUUUGUUGCCUGGGGCUCCUCUCAAA | |
| | AGUUACACCUCUUUGCAUCGUUUGGCAAAUAGAUCAUAACAGUAGUCUCA | |
| | AGUGUAUUAUAUGUUGCACCUCAACAUCAAAGAGGUCUGCUGAACAUUGU | |
| | GAUUCUCUUUCAAAGCUAGGAGGCAAGAUAGUAAUUUGCCCUUUACUUUG | |
| | GAAUACAUAUCCCAGGAUGCUCAGAACACUCGGACCCUAAGAUGUUUACC | |
| | UAUGGGGCAGCCUCUUGAAUCUUCCUCUAGAACACACGUUUCUUUCCAAA | |
| | GCAUCCAGAAUACUUGCCACUUUUUGCACAUCAGGCACGGUUAACAUGAC | |
| | AUCCUCAAUAUAGUGGACCAGUGUGAUGUUCUGCAGAAUGUCCAGAUGAC | |
| | UCAAGGCUGUGCUGACAAUGUUAUGACAAAGAGCAGGAGAAUUAACACAG | |
| | CCCUGGGGCAAGACUGUGGAUAUACACUAUUAUCUGUUUAUGUGAAUGAG | |
| | AACUUCUUCCGAUCCUUCUUUCUGAUAAAAAUGAAAAAGAAAACAUUUGC | |
| | CAGUUCAGUGGCGACAUACUUUUGGUGUGCUUGGUGACUCUUGGUUAACA | |
| | GGUGGUCCACAGUUUUUGCAAGGCCAGGCUCGGCUCGUGUAUUAAAUAGG | |
| | ACCCCAUUAGGGACCACCAUUCCUGUCUCCUUGAAGACUUGGAGGGUGGC | |
| | ACUAAUUUUUGCCAUUUCCUUGAGAUGCUGUUAUGGGUUGAAUUAUAUCC | |
| | CCAAAGGAUAUAUUGAAGUCCUAUCCCCUGGUAUCUAUGAAUGUGACCUU | |
| | GUUUAGAAAUAGGGUGGAUGCAGAUAUUAUUAGUUAAGAUGAGGUCAUAC | |
| | UGGAAUAGGGUGGACCCUUUCCCAGUAUGACUGGUAUCUUUAUCAGAAAA | |
| | GGGAAAUUUAGACACAGCUAUAGACACAGAGGGAAGACAGUUAUGUAAAU | |
| | CUGGAGGCAGAGAUUGGAGUUAUGCUGCCACAAGUGAUGGAUUGCCUGGG | |
| | GGCUACCAGAAGAUGGAAGAGAUGAGGAAGAAUCCUCCUUCUAAGACUUU | |
| | GGAGGGAGCAUAGACCUGCUGACACCUUAUUUUUAGAUUUCUAGCCUCCA | |
| | GAACUGUGAAAUAAACUUCUGUUGUUUAAAGCCACUUAGUUUGUGUACUU | |
| | UGUUAUAGCAGCCAUAGGAAACUAAUACAGAUGCAAUAUAUAUACUAUAU | |
| | AUAAUAUAUAUGAAUACAUAUACAUAAAUAUAUAUACUAUAUAUAAUAUA | |
| | UGCAUACAUAUACAUAAAUAUAUAUAAUCAUAUAUAUUUAUAAAUUCAUU | |
| | AUCCUCGUGGAAGUGUCUUACUGGCAUAUCCAGGAUCAUUCUCUGGGCCU | |
| | UUGCUACAACAACAGCUAUAACUUUACAAUUCAAUGAACAAAGGGUUCUG | |
| | CCAACUGCUAAGUAUGCCCAUCCCAAUUGUACAUUUGGAAAUUGUGCAAA | |
| | GGCCCACCCGUACCCAUUGUGAGACAGACAUGGUUUGGACUCCGGUUGCU | |
| | AGUUGACUUCCAUAUACCUCUACUCUUAGCAGGAGGGUAAUGUAUUAGUC | |
| | AGGGUUCUCUAGAAUCAUAUAUAUAUUAUAUAUAUGAGAGCUUAUUAUGU | |
| | AUUAACUUACAUGAUCACAAGGUCCCACAAUAGGAUGCCUGCAAGCUUGA | |
| | GGAGCAAGGAUAGCCAGUCUGAGUCUCAAAACUGAAGAACUUGGAGUCUG | |
| | AUGUUCAAGGGCAGGAAGUAUCCAGCAUGGGAGAAAGAUAGAGGCUGAGA | |
| | GGCUAGACCAGUCUCUCCUUUUCACGUUUUUCUGUCUGUUUUAUAUUCAC | |
| | UGGCAGCUGAUUAGAUGGUGCCCAUCCAAUUAAGAUUGGGUCAGCCUUUC | |
| | CCAGCCCACUGACUCAAAUGUUAAUUCCUUUGGCAACACCCUCACAGACA | |
| | CACCCAGGGUCAAUACUUUGUAUCCUUCAAUCCGAUCAAGUUGGACUCAG | |
| | UAUUAACUAUCACAGGCAAUGAUGGUGUUUGGGAUCUUUGGCUAUCAGCA | |
| | UCAAUCUGUAUUCACGUGUUUGGAAAUAUCUGAGUAAUUUACGGAUUGAU | |
| | CGAUGGUGUUGCAACAUCUUUCUUGUUUUUCAGUUGAUGUGUUUUGGGUC | |
| | ACAGAACUGGCUCAGGCCUAAAAGCUGAGCAUGGAAAUAAGGCUUUUCAC | |
| | UGAGCAUAGCUGACCUCAAACUUCUACUCUGCCGUCACGUAUCUCUUUUG | |
| | AAUAUUCAUAUUCAAAUACCCUUAUUGGCUGCCUAUCUAUCUUGCCCCUA | |
| | GGGACAAAAUGUUCUAUGAGCCCUUUCCAUAAAUUCUGUGGGUCAUGCCU | |
| | UGCUGACUCAUUCUGAACUUGCUGUGGAUUAUAGCCUUCUGUCCACCGCU | |
| | CUUCUAUUGGGUACAUGCUGCCACCUAGCCUCUGCUCUUCUCAGAUCCUA | |
| | UCAUCUCCAUUGCUAUUAGGGAACCCACUACCAUAACAAUAUCUCACAUC | |
| | AUUAUUCCCAGGGGUCCUUCUGACCUUAUUGCCUUGGGCAUGUCUUAUGG | |
| | GCCUCCCUAAGGAAUAUGGUCGGUACAGGGUUUUCUGUCUCAUAGAACGG | |
| | ACCCAUUGCUUGCAUGUUGCUUCUGUGAGCCUUUUAAGCCCUUCCUCCAU | |
| | CAUUUGCUACAGCAAUUCUGGCAUCUCUAUUUUGUUUACUGUAGGCCUUU | |
| | CCUUUUCUCAGGCUUUCAAAGUUAAUUGUAUCAGCUGAUUACAGCAAUCA | |
| | CUGGGGCUCUUGCCAGGGCAUUAAAUCCUGUGUCACUGGAAAGUGCCACU | |
| | AUAUUGCAAAACACUCCCUAACACAGCUGUAUAGCCUAUCUUCUCUUGAU | |
| | UUAACCCCCUGAGGAACCACUUCCAGUUCCUGAUGGUAACAUGUUAUCCA | |
| | UGUCUUGAUGUUUUUAGGCAUAUAAUUUCUUUUCUGCUUUACCUGUCCCA | |
| | ACACUUUUGAUCAGGUUAUGCUGAGAUUUUACUCUAAUUAUGAACCAAGA | |
| | GAGGAAGUGGAGCAAGUCCUGAGAAGGUAAAGAAAUGUUAUCUAAGAUAC | |
| | CUGCCUCACUUGAUACCUUUGUAUAACUUCAAACGUGUGUGUGUGUGUGU | |
| | AGCUCUGUCUUCCAAUAAGAGAUAAUUGGUCACAUUUUCAGGUGCAUAGG | |
| | ACUCAGGAUAACCCAAGAUUUGAGAUUCUUAACUGUGUCGAUACAGAUAU | |
| | CCCCAUCCAAGCAUCAGGGCCCCACUCCCUUCAGGGCUCUAAGUUUGUGU | |
| | AAGAGACCUGCCUAGACUGAGAUCCAGCUUUCUCUGAUGCACUGCUAACA | |
| | AUUAAUUUUGUUUUCUCUUGUUCUUCAACCGUGGCUGCCAUCCAGGUGCA | |
| | GAAGUGGAAGUUUUAUUUAAAUAGUGCCUAGGAGUUCCUGUUACUCUCAC | |
| | UUUGCUAAUUUACUUUGUUGAUUAAUAGACUUGAACCUGUCAUUUUCUCU | |
| | CUUCAAUCACUGGUGCUGAGCAGUAGCCAACCAAUUCCACAUUCUUAUAG | |
| | UUGUUAUUUCUUCAUAUCUCUAAAACACCCAGAGUAUUGUCGCAGCUAUU | |
| | GCACCGCUUCCACCUGUAUCCCAUCACAGGACAGCAUAGGUGAAAGCCAG | |
| | UAAAAUGCACCAGCAUUCCAAGGACUAUCAACAUUCCACCUAUCACCAGC | |
| | AAUAGGGUUCUCAUUAUCAUCCAGCUGGAAAAUGAUCCAACUCCAGGAUC | |
| | CCCAUCUUUACAGUCUGUCUCUUAGAAGUGCUUCUUGUAUCAGUGGUCAU | |
| | AGAUUGGGUUCCAUUCAGGUUAGGGAUCCUUGUUUAAGUCACUUAUUGGG | |
| | GAUGUGCUCAAUAGGGGAGUGAGGGAAGCAGAAUAGGAUGGGGAAAACGC | |
| | CAAGCAAGAAUGUGGUCUCAGGAGGGACUAGCUUCAGUCUGAUCCUACAG | |
| | GACACUGGAGCACAAAUUACACAACAGAACUAGUCGUACAUAGAGAAAAA | |
| | GAGGCUGGCCUUGUGUAUCCCAUGCCAGGAGGUCAUUGGCUGAGUCAACC | |
| | CUGGAGAAGGGAGGGAAAGUGGUGUAUCCACCCAGGCAAAGUGGCUUCCA | |
| | UCUGGUUCAGGGCGAUUCUCUGGAAUAGGGGUGAGCUGUUUGUUGUUAUC | |
| | UCCAAAAGUCACAGCAAAUGGGAGAUGGUACAACCAGAAAAGGGAAUGUG | |
| | GGCAAACACCAGCCACAUUCACCACAAUAUUACAGAGCUAUUAGGAACUA | |
| | UAAUGAUCAGCAUGAAGUUUUUCUGUGUCCUAGGUAAGAAAUACAUUUCU | |
| | UUAAACCGUCUGGAAAUCUUCUAUAGUCUUGGAUAAGAAGGUUGUGUUUA | |
| | UCUUUCCUCCUUUUUAGUGGAAUUAUACUCAAUCCCAAAUGCCCAUUCAC | |
| | UAUGUUUAACAAGGGACCGUCACGCAAAUAGCAUCUCUCUUCCUGGUCUG | |
| | CUGUCAACUCUUCAAGUCUCCACAUCAAAAAUGUUAUUCCCUACUGAGUU | |
| | AACUUUACUUUUCUCUCUCCUUACCUUUGUUCUUGCAUAUAAUGGUUGCA | |
| | GUGAGGGAUUAUGAGAACAUCUCCUUUCAGAUACGUUGUCACAUUCAGAA | |
| | UUGUGUGAUCUAGCAUGAGAAAGGCUUAACCUUUUUCCUUUCCAUGUGCC | |
| | AGUGAAAAUUUCUUUAUACUAAUACACGUUAUUUCUGAAAAGGGGAUGAG | |
| | AGAAAGGGACAAGAGAAGGGAUGAUACAAAAGUAAUGUAGAAGUGGCCAA | |
| | GAAUAAGAUUUGGCUUUCCUAUUUUUUUAGUUUGAGUUUGGAAAUUUAUG | |
| | CAUUCUUUUUGUUUGUUUUUGAUCCAGUAAAAAAAGUGUCACCUCUCGUC | |
| | AGGACUUCAAUAGUUCUGAAGGGUCUGGAAGUAAGUCAUUGUCUCCUGAG | |
| | UUGAUUUAAGGCAGUUUAUGAUGAGAGUCAUUCUGUUUUUUAUUCCCUAA | |
| | AACUCUGAGCAUACUGCUGAGUGAAUAGUCUGUGCUCCACAACUACUUGA | |
| | AAAAUGGUGAUGCAGGCCAAAGUGUUUUGAAAACUGUAAAAAUCCAUGCG | |
| | GAAAUGAGGUAAUAUGAUUGCUUUAAAUUGACCUCUCCAAAGGCUCUUUC | |
| | ACCUUUUACCUGUUUUACCUGUUUUACCCCAUAUAUAGAUCCCAAUGUUC | |
| | UUCCUCCUACUUGAUGCAGAAUGCCUGUUUCAUUUUCUCCUUUUUAGCCA | |
| | UAAAAAUUCCAAGUAUAAUUCUAGGGCUUGGUUUGAAUCCCACUGUGAGG | |
| | AAACAAAUCCCGAUACUCUAAGGUUUGGCAAUGGAGGAUGAAUAUCCCUU | |
| | UAGGGAAAACGAUGGCUGAAACCUGUGGGUUUCACUCGUGUCUACUGAGA | |
| | UUCUCCUAAGGGACCUUCUUUUCUUAGGACAGUGUUUUCAAAGUGUAGUC | |
| | CGCACGUCAGAAUAUUAGCAUUACUAGAGAACUUGUUAAAAAUGAAAAUG | |
| | AUUAGCUCUACCCCAGACCUAUUAAAUUUUGAACCCUGAUUGUAGGACCC | |
| | UGCAGUUCAUGUUUCAGUAAGCCUUCCAGGUGAUUGUGAGGCCCGCUAAA | |
| | GUUUGAGAACCAUUGUCCUAGGGAAUCACUUACUGAUCACUUUUGAUACC | |
| | ACCCCAUUAGCUUCAGGAAGUCCUCCUCAACUUAGCACUUUACACCAAAU | |
| | UAACCCUUAAUUGUUGUGGCACAGUCAAAACAUUUAAACUAGCUUUCCUA | |
| | CCUAAUGCCUUCUCCAUCAUAAAAAGAAAGUGAAUUCCUAACAUUAAUGU | |
| | AUAAUUAGGCCAGGCGCGAUGGCUCAUGCCUGUAAUCCCAGCACUUUGGG | |
| | AGGCUGAGGUGGGUGGAUCACCUGAGGUCAGGAGUUUGAGAUCAGCGUGG | |
| | UCAACAUGCUGAAUCCCCAUCUCUACUGAAAAAAUACAAAAAUUAGCCAG | |
| | GCAUGGUGGUGUGUGCCUGUAAUCCCAGCUGCUCUGGUGGCUGAGGAGGG | |
| | AAAAUUGCUUGAACCCAGAGAUGGAGGUUUCAGUGAGCCAAGAUCAUGCC | |
| | AUUGCACUCCAACCUGGGUCACAAGAGCAAAACUUGGUCUCAAAAAAAUA | |
| | AAAAUAUAAAAAUAAAAAUAAACCCACCAGUUUUAUAGUCAGGCAAACUA | |
| | CACUUUGACUCCUGCUUUUCACUCAACCACGUUGAUAGGAAGGCUCUGCA | |
| | UCUGUUAAAUGAUGGUAAUAGUAAUAUCUACCUUUUAAGACUAUGGUGGA | |
| | AAUUAAGUGAGUUGGUGAAUGGAAAGCACUUAGCAAAACGUCUGACUCAU | |
| | AGGUGGUUUUCAGUAAAUGAUAACAAAUCAUAAUUUAGCUUAGUCAUCAU | |
| | CAUUUCAUAGCCCCCUAUCUGCAGUGGGUCUCAGAGACUAGCUCUCAGGC | |
| | CACCUCCACCGGGCAUGGAACAUUUUUUACCUUUGGAAUUUAGAGGCAAC | |
| | CCUAAGCAUGUCUUGGGCCCUUAGGGUGUAAGCCAAGAGAGAAGCAGGAA | |
| | UUCUCUUCUGUGUCUAACUUCCACUGUCCAAAUUCUAACUUCCACUGUCU | |
| | UAAGACCCUUCUGUCACCUAGGAUAUUGUUUUUGGGAAACAUCAGGCCCA | |
| | UUCCCAACAUUUACAGGGACUGAAGCAGGAGUACAAAUGGAAGGCCCACA | |
| | CAUCAUAUAUGUAUUUAUAAUUUACAGAGCAAGCUGAAAAUGUUUUAUAC | |
| | UCUGGGUAGGCAGGAUAUCAGCCCACACAAUCCCAGGAAGCUGUGACUAU | |
| | GUUACAUUAUGAGAUAAAGGGACUUUGCAGAUGUGAUUAAGUUCAGGGCU | |
| | UGAGAUGGGGUGGUGAUCCUAGAUAAUCUAGUCCGAUGAAAUCACAAGGG | |
| | UCUUUAUAAGAAGGAGGCAGGAGAGUCAGAGAAGGAGAUGUAACAAAGGA | |
| | AGCCAAAAUUGGAGUUGUGCAAAUCCAUGAGUGAGGUAGUGGGCAGUCUC | |
| | UAGAAGCUGUGAAAAAGCAAGAAAAGGGAUUCUUCCCUUGAUCCCCAGUG | |
| | GGAGUGCCUUGCUGGCCCAUUUUAGACUGCUGACCUUCAGAACUGCUCCU | |
| | UAAUAUAUUUGUGUUGUUUUAAGUCAUAAAGUUGCAGCAAUUAGUUACUA | |
| | CAGUGAUAAGAAACUAAUAAACCUCCUACCUUCAUAACAGUACAAUUUUU | |
| | AAAAAAUGUGUACAUAUGUUUUUAUAAGACUGAAAGUUGGCAAAAUAUCA | |
| | AUGGUGAUUGAAUUUGAUUAUUAUUUUGCAUAACAAAUGGUCUUUGAAUG | |
| | ACUCAAUAAUAUUUGGGUGAAUAAUAAAAUAAAGAAUAAAAUAAAGAUAC | |
| | AAUUUAUUAUUAUUAUUUUCUGUUUAAGAUGGAGUCUCGCUCUGUUGCCU | |
| | AGGCUGGAGUGUAGUGGCAUGAUCUUGGCUCACUGCAAUCUCCAUCUCCC | |
| | AGGUUCAAGUGAUUCUCUUGCCUCAGCCUCCCAGGUAGCUGGGAUUACAG | |
| | ACAUGCCACCACACCCAGCUUAUUUUUGUAUUUUUAGUAGAUACGGGGUU | |
| | UCAUCAUGUUGGCCAGGCUGGUCUCGAACUCCUGACCUCAAAUGAUCCAC | |
| | CUGCCUUGGCCUCCCAAAAAGCUAGGAUUACAGCUGUGAGCCACUGUGCC | |
| | UGGCCUCAUUAAUUAUUUUUAUCUCAUAACACUUAUUUUUCUUUCCUGUA | |
| | UUUCAGCACAAUCAUGAGUUAUAUGGUUCAGAGAACUUGUUUCUUAUACA | |
| | CAGUAUUGCCAAACUAGGCCACCAUACUUGAAUUACUGUAGUUCCUAGCU | |
| | AAUAUUUUUAAAUUAUUUUCAGUCUGGAGAAACUUGGUUCUACUGAGGCA | |
| | GUAGCAACCUAAUUGUCAUUAAAACUCUUAAAGAAUACUGGAUUAAGAAA | |
| | UAGAUUAUUAAGUUAUAUAUCACGUUCAUGCAUUAUAAGGGGAUUGUCUA | |
| | UGAUGCAUGAUCUCAUCAUGAUCUUAUCAUGAUCUCAGAAAAAAAUUACC | |
| | AAGUAUUUAACUUAAGCAAGUGAUUACUGUUAUGUAUAUUUUGCUUUUCA | |
| | ACACCUACAAAUCAAUAUCUAGAUGCCUAGAGUUUUACUUAAGUUUCUUU | |
| | UUUCCCCCAAUCUUUUCAAUGGUGAAAUAUUAGAAGAAAAUUUUAAAAAC | |
| | AGAAGUAUGAUGCUUAAUUUGAUCAGUUCUCUCACUACAUUUGAUACUAU | |
| | UUCUAAAUAAGAAUGACCAGUCUGCAGGUUCUCAUUUGUUUGUGCAUGAA | |
| | UUCCUUUGUGUUCAUAUAAAUAUUUUCUAUUUCCUUUUUCUAAUUUUUAA | |
| | AUAUUUUAUUGUUGCAUCUCACAUGUAGUUUAUUUCAAUUUAGAAAAAAG | |
| | UGUUCAAAAUAUUUGCAAAGACUUUGUAUUUUUUUUAAAAUGAAGUAGCC | |
| | AAACCUAGAUUGUUAGUGUUUUUAUAAACUUCUGCCAUAUAAUAAGUGGC | |
| | AAGCAACAAUUCAUACUGCAUCAAUUUGUGAUUCUCUUGAGAAUCGCAAA | |
| | CAAGAACACAGAGACAUAAGAAAACAGACGCUUGGCACUUGUGGAAAAUG | |
| | GUGUGUUAUUAUACAAGAAUCUUCUGCUUUAGUUCUGUCAGAGAGGGCAG | |
| | AAUUGAGUAAAUACUUUGUCUUUUCUUUUACCAAAAUCCUUCCCUGUACC | |
| | CUGGAGCCCUGUCCGUCCAUCCAUCUCAGAUGUUAGUUUCAGCACAGUCC | |
| | CGUAAACCUUCAGGCACUGACACAUAAAGACCUUUUGUUCUGAGGACUGG | |
| | GAAGAGAGAUGUAGAAAAGUGUUUCCCUAGGGCGUGGACAGUUUAUACUA | |
| | CAUCCACUAAAGUCAAGUGCAGUUCUGAGGGAACCCUUUUAUGGGAUUUU | |
| | GCAUUCUCUACCCAUAGAAAUGUGUCUUUCUCCUCUUAUUGGAAAAACAA | |
| | CAACAAAAGCAUAGAAAUAGUAAUAGAACUCACCGCAACAGCAUAAUGCA | |
| | CCAAGUGGCACAUGUGUGCACACUGACACUCACACAAGUGAAAAACUGAA | |
| | GCAAGUCGCCCAAAUUUGCAUGGGUAACAGAUUUGGGAUUCGAGCCCAUG | |
| | UCUACUUCACAUUGAGUUCAUUCUGGUACGUCAUCUUGCUUCCUCAAGAG | |
| | CAGGGUGUUAUCCUUUCUCUGGUUUAUCUAGCAGCUUACCUGUAUUUGUC | |
| | ACUCAAAACUGACUCACAUUUCACCUCCUGGGGAAGGUGCCCCAAUUCCC | |
| | UUUUGAACUUUCUACAGUAACAUGCUCUUCAGUCAUUUAUAGGGUUACGU | |
| | GCUGUUCUCACGCUGGCCUCUGAUUCUUUGAGGACAAGGACUGUGUUUUA | |
| | CUGCUUUCUGCAUUUCUGAGGCCUAAGAGAGCGCCUGGAAUAUAUUAGGA | |
| | GCUGAAAUAUGGGAUAAUAAGAAAGCUCUUAAUUUUAAAAAAGAAAUAUG | |
| | CUACCACUAUAAUUCAGCAUAUUAAGCAAUAAGUAUGGACAUGAAAUCCA | |
| | GAAGUCACAUAAAUUCAUCUUUAGGUCCCAGAUUGAUAGCUGCUGGGAUA | |
| | UUUUUAGAGAAUUUUGAAAAGAUUCCACAGUUUUCUUCAGCAAUUUAUUC | |
| | AAAUAUGGGCCAUUCUAUACUGUCAGCAAAUUCUCUCUGAUAUUUAACCU | |
| | GAAGCAGUAACCUAUAAAUGGUAUGCUGAGGCUCUGAGAGAGCUGGUAUA | |
| | AUUGCCAAAGUUGCAGUCUUACUACUGGGAUAUAUUUAAGGUUUGGGAAA | |
| | GUCUUAAUUCAGCACAUUUUGAAGAGCAGUAUUUUUAGUUCCUUGGGUGA | |
| | AUGGUCCUGUUUUAAUUCAGGAAACAAAAGGUGCUGGUUUAGCUAACAGU | |
| | UUGAUUUCAAUCUCUGAAGCCUUUCCACCAAUUAUUUUCUUUUUCAAGCA | |
| | AAAUCAAUCUUUCCUCCAUGUUCUCAAGUCCAAUUUAUUUAAAACACACA | |
| | CGUACAUGUCUGCAGGCACACAUACAUACACACACAUAGUAUUACAGUAC | |
| | AACUAUUAUAUACUGUUUUAGAUGUAGAUAUAGAAAGAAGUAUAGAUAUA | |
| | GAUUGCGGUUUCUUGAGUUUCUAAGAGUUUCCUUAUUGUUUGCUUCUCCA | |
| | GUGACUUUGCAAUAACCUAGCAAACUUCCAGUAAAUGUUCGCGAAAUACG | |
| | UGAAUAGUAAAUGAGAGAAAUGAGGUGGAGCUUGAAAUAAUCCUGCCACC | |
| | UUUGUUCCUAUCACUGUAAAUGUUAUGUAUACCUGUCUCUUUUCGUAGAA | |
| | AGUCUUGCACAUAUGUACCCGGAGAAGAGACAGUCAUUGGUGUGAUUGCU | |
| | GUCUUAGAGGGUAUGCUCAGCUUGAACUUUACCAGGAAACAACAAAUUAU | |
| | UUUUGAAAGUGAUUGAAAAUUUAUACUCCUACCAGCCAUGUAUAAAGGUU | |
| | CCUAUUUAUCUGUGUACUCUCUGAUCUUUGAUAUUGUCAGACUUAAUUUU | |
| | UGCCAAUCUAGUAGGUAUGUACAGGUAUCUCAUUGUGACUUUACUGAAGA | |
| | CUGGACAAUUUUCAGAUAUUUAUUUGCUCUUUAUUUUUUUAUUUUCUGUG | |
| | ACAUCUCUGUUUAGCUUUUUUGUUCUCUUUUGUUUUUCUUCUUUAUUACU | |
| | GAUUUGCAGGCAUUUUUACAUUUAUGUAAAUACUAAUAUUUUGUCUCAGG | |
| | CUGGCUUGUCUUUUUACCCUCUUUCUGGUGCUUCUUGGUGGAUAACAUUC | |
| | UUCAUUUUGAUGUAGUUGAAUUUCAUCACCCUUUUAUUUUAUAUGGUCAA | |
| | UGCUUUGUGCUUAAAAAAUCCUUUCCUACCCCAACUUUAUAAAUAUCUUU | |
| | UCCUAUAUUUUCUCUAAAAGUUUUAAGGUUCGCCACCAUUCGUUGACUGA | |
| | UCUUUCUCUCAGUAAUUUCCAAUGCCAUGUCUGUUACACAGCAAGCUAUC | |
| | AUUUAUGCAGGGGUCUGAUUCUGGGCUCUCUAUUUGUUUGGCCUGUGUGU | |
| | CUGUCCCUGAGCCUAAUUAGUAAUUUAUAUCAGUAAGGCAUUUUCUCCUA | |
| | CUCUGUCACUCUUCAGAAAUGGUGAGUUCUUCCUGCUCCUGUACUCAUCA | |
| | UGUAAAUUUUAAAAUCACUGGUUAAAUUUUAUGAAAACCUGUUAUAACUU | |
| | UCAUUGUAAUUGUAUUGAAUUUAGAAAGCCAUUUCUGGAAAUUGGCAUUU | |
| | UUUUGGUAUUGUGUCUUCCUAUCUAUUAACAUUCUGUAUUUCUUGAUUUA | |
| | UUUAGGUCUGUCAAAGUCUUUAGAAAAUAUUUAUAAUUAUUAUUCUUAGG | |
| | UCUUGCUCCUCUUUGUUACAUUACUUCACUUUUGCUACUAUUGUAACUAG | |
| | UAUAUUUUAAAAAUAUAUAUUUUCAAUCUGUGGCUGAUCAUGGAAAUAAA | |
| | AUUAGAUUUUAUAUUUUGACAUUACAACCAGCUGUUUGUUAUUGAUUAUU | |
| | AUGCAUAAUAACUUGUCUUGAGAAUCUUUUGUGUUUUACAUAAAAAUCCA | |
| | UUUUCUGCAAAUAUUAACACUGUGUUUCUUCCCUUCUAAUCUUUACUCCU | |
| | UUAAUUUUUUUCUUGUAAUUGUAUUGGGUGUUUAGUACAGUGCUGAAAAG | |
| | AAGUGCUAUUAUAGUCAUGCUCGUCUUGUUUCAUAUUGUACAGGGAAUUG | |
| | CAAUCAGAUUAUGUCUGUUGUAGGCUUUUUAUAGAUGCAUUUGAUUAGGU | |
| | UAUGGGCGCUUUUUGCGUUCUUAGCUUUAUAAGAUUUUUAAAAAUAUGCU | |
| | UGUGUUGGAUUUUUUUUAAAUUAUAUUUUUCCCUGCACUUAUUGAGAUGG | |
| | UUUUUUUAAAAAAUUUAUUGAUAUGGUGAUAAAUAUAUUUUCUAAUGAUA | |
| | AACUGCCUUUUCCUUCCUAAAAUAAGCUCAACUUGGUCAAGAUAUAUUAU | |
| | UAUGGUUUUAAAUUUAUAUUUGGAAAGUUUGCCUAUAUUUUAUUUAAUUU | |
| | UUUUGCAUCUAGUUUCAUAAGUGAAGUUGACUGAUAUUUUUUUCCCACAU | |
| | UGUGGUAUUUUAAAAAGUAUCAAUGAUAUGGUAGGCUCUUAGAGUGACUU | |
| | GAAGAUUGUGCCCUCUUUUUAUCUUUUGGGGAAGAGUUUAAAAUUUAUAA | |
| | AUAAUGUGGUAGGAUUCACCUAUACAAUAUCUGGGUCUGAUAUUUUCUUC | |
| | AUGGGAAGAUUUUUUAAAACUGUGAUUCGUUUUUAACCUAUUUAAAGCCU | |
| | ACUCUUUAUUCUUAAUACUGUUUUAAUGUUAUGUUUUUUCUAAUAAUGUG | |
| | UCCAUUUUGACUAAGUUUUUUAGUAUGUUGGUAUAAUGUGAUACACACAC | |
| | ACACACACGCGCGCGCGCGCGCGCACACACAUACACACACGCAAAAUACC | |
| | AUAGUGUUACCUUAUCUUUUUAACCUGUUUUAUCUGCAGUUGUAUAUCUG | |
| | CAGUAAUCUGCAGUUUUGUAUCUGUAGUUAUGACUUAUAUAGUUAUAUUU | |
| | AAAACUUCUUUUGAAACAAAUGUUCGUAACUGCAUAUCAGAAAAGUAUUA | |
| | CAGAAGGUAUUUAAUCAUUUAAAUUUGAGUGUGUUCUGUGCUUCCCAGAU | |
| | GCAGAAAUAAAAUAUUUUCUUUUGUUUUAUUUUCCAACACAAGAGGGCAG | |
| | CAUUGCUGCACUGUUAAUUUCCUGUAGGCUAACACCGCUGUGCCUUGUGU | |
| | AGGUUUAAAUAUACAGCACAUCUCUUUCAAAUGGAUAAUCAUUUGAGGAU | |
| | UGCAUAAAUGUGGACCUUGUCUAUUUUUCACUUACAUGACAUUCAUUGAA | |
| | AUUUAGUUUUGAAAACAAUACUCUCUGAUUAGCAUUUAUACAUGUUUUAU | |
| | AUUUGCUUCAUUGUUUCUGAUUCUGUAACAAUCACCUCAUUCUGAGAAGA | |
| | CUAGGUACUGUUGUGUUUGAUAGUGUGUAUGUAUGGUAAUUUUCUAGAAA | |
| | UCUGAAUUUUUUUAAAUGGGCUUUUCAAUGAUAUUACUGAGAAAUAGGUA | |
| | GGGUACUAUUUUGACAUAAAGGAUAAAGAACCCACAAAGUCCAUGAGCUU | |
| | CCUGUCGGAUAUGAGAGAUUAAAACUGCUGCUGACUACACCACCAGGUCC | |
| | AUGUCAAUUAACUCAUUUGCAGAUGGGAGGUGAGAGUGCAAAAAUAAUAU | |
| | UAACAUUAAUAUUAAGUACCAUUACUGGCUACUCAUUAUAGCUUAGAUUC | |
| | UUUAUUUGCUCUACUUAGAUUUAUUUUUAUAACAUCAUCCUGUAAAGUAG | |
| | GUAUUAUUACCCCCAUUUUUACAGAUGAGGAGGCAGAAGCUUGGGUUGGC | |
| | UAACUCACCCCUUAGUUCAUGAGGGUCAGAAGAGGGUGCUGAUUAAUAAA | |
| | UCCCCAGGCUUUCUCACUACUGCACACUGCCUCACCAUCAACAGCAAAAC | |
| | AGGUUGAAUUCGACGAUUCAGUAAAAAAGUAAUUUUGAUAGUUGAGUUCC | |
| | UUUAUCCCUUACCACGUCUAUUUCACAUAUAUGAUUAUUAUUAUUUUGGU | |
| | AGAGAUGAGGUCUUUUUAUGUUGUUCAGGCUGGUCUCAAACUCCUGGGCU | |
| | CAAGCAAUUCUCUUCCUCCUGAGCCUCCUAAACUGCAGGGAUUACGGGCA | |
| | UGAGCCACGUGCCUGGCUCACAUAUUUGAUUUGGUCUAGGCUAUCUUAAC | |
| | AAAAUACAAAAUGCUUUGUAAAGUCUACAUUUUAUUAUUGCAUUUGCUAU | |
| | UUUUAGCUCUUUAUGUCUGAGCAAUUGUUUAAUGCCUGUGCCUGAGAAUC | |
| | UUUUUCUCAACAGGGGAACUGCGGGGGAGAUUCCAGAAGUUCCUUUUAGA | |
| | GUUGUUGUUUCACUGUAUCAGUGCAUCGGUGACUGAGAAGGAAGUAUUUG | |
| | CUUUUGUGUGGCAGCGCAGUAAAUUCGCAGAACACAGUUCCAAGAAGCCC | |
| | AAGGCACUAGCAAGAAUGAUAGGUCACCAUCUCCAUUAUCUGUCGGUGGU | |
| | GGAUAUGGAAGAUAGCAUUCUAUAUUAACUUUUGAGAAAGAGAAAGUUAG | |
| | GCAAGAAGAGAUUGCUGAAAUAGAUUGUCUGUGAUUGCUGAGGUUGAAAG | |
| | UUUUUUAUUCUUGGCCAAGGAAUGUAUCUUUUGGGUUUAACAUCACUUUA | |
| | GGAGUCAGACAGCCUUGAAUGUUAGACUGAGCUUUGCAUUUUUUAGGUCA | |
| | UGACUUUAGGCAGAUUCCCUGAGCCUCAGAUUCAUCAUCUGUGAAAGAAA | |
| | GAUAAGACUAAUACCAAUUUAUCCAGGCUAAAAAUAUAUAUAAAAAUAUA | |
| | UUUUUUCAAACAUACACAUUCAAAAAUAAUGACCAUAUUUUUAAAUGCUA | |
| | UCCUACGUUGUGUCAAGGGCACCAGUUUCUAACUUAGAGACAUUUGCAAU | |
| | UUUGAGCAGUGUCUUCUGAGGCCACUGGGUAUCUCUAGAAAGCUUGAAGU | |
| | UUAGGAGUAAACAGAAAAAAGCUUGUGAACAUGCUUUAUAAACUGUAAAG | |
| | AUUUGUAAAUAUAUAAAAUCAUUAUAAUGGCUCCUCUCUCUGAACUCAAC | |
| | UUCCACUGGAUACAUGGCAUAUACUUUUGCAUGUAUCAACUUAGCUCAAA | |
| | UCAAUAAAAAACAUUUUUGGACUCCUGGUAAGUCCAAACAUUUAUUCAUU | |
| | AAAAAAAUUAUUAGGGACCUAAAUGUACACUGUUUUAGGCACUGGAGAUU | |
| | GGACAAGAAACAAAACAGAUGAAACCUUUGCCCGUGGAGCUUGUAUUUGU | |
| | AGAGGUGACAUAUGUGGAUCACACAUAGGUAGGAUGUUUCAUAAUAAUAU | |
| | AGAGAAAAAUAAAGUCUAGGCAAAUGGCUAGAGAAUGGUGGAGGCUUGCU | |
| | ACUUUAGAUGGAAUUGUCCAAAAGCCUUCUUUGAAGAGGGAAAAAACUGA | |
| | GCAAAGACUUGAAUGAAAGAAAGAAACCAGCCAUACAUCUGUCUAGAAAA | |
| | ACUGAGUUAUUGGCAGAGAGAACAAAAAGUCAAAGGCCUUGAAAUGAGGA | |
| | UGUGUGCCUGGUAUGUUUGCAAAAUGGCAGGAAAGCUAUGGUAAACCAUA | |
| | CAAAUUAUUGGGUUUUCCAUGUAAUCCAUUGUCUCUUUGAGAGACGAUAU | |
| | CUAUUAUAAUAUUAUAAUUUUAUAGCUCCUCUUUUAUAGGUGUUCAUAAU | |
| | AAGAAUAAAGAUAAUAAUUAGUACUUUUUGAGUACUUGCCAUGUGCUAUA | |
| | UUUGUCCUUACAACCCUCCCAAAUAAAUAUAAUUAUCCACCAUUAUACAG | |
| | AGACUCUAAAUAGCUUUCCUAAGGUAGCAUAUUCAAUAAGUUGGUGAGGC | |
| | AACGGUUUGUCUUAUUUCAAGUGCAGGGUGCAUAAUAACUCAAUGGGAAG | |
| | ACAGAUGAGUGAAAAAUGCGUAGUAAUGUCAGAGGGCUUAUGAAAGGUAC | |
| | CUAGGGAAGGUGUCAUGAAGUUACUUAGGGAAGGUGUCAUGUUAUGCCAU | |
| | AGAGGAUCAGUUCCUUAAGGAAGGAGAAGAUGGAUUGGGAGUGGAGAGAG | |
| | AAUUUCCUGCGAGAAACAAAGGCAUGAAGGCUUGAAAGAGCAUGGGGUAC | |
| | AUACGCAGUAAAUAACAAACAGUUAAGUGCGGCUGAAGAGUCUGUAGCAU | |
| | GCGAUUUAACACAGAUGCUUCUCCACUUAUAAUGAGAUUACAACCUGAUA | |
| | AACCCAUUAUAAGUUGAAAAUAUUGUUAGUCGAAAAUGCACUUAAUAUAC | |
| | GUAACCCACAGAGCAUCAUAGCUUAGCCCAGCUUACCUUACAUGUGCUUA | |
| | GAACAUUUACUUUAGCCUACGGUUGGGCAAAAUCAUCUACCACAAAGCCU | |
| | AUUUUAUAAUAAAGUAUUGAAUAUCUCAUGUAAUUGAUUGAAUACUGCAC | |
| | UGAAGUGAAAAACAGAAUAAUUGUAUGGGUACUCAAAGUAAGGUUUCUAC | |
| | AGAAUGUGCAUCCCUUUCACAACUUCCUAAGGUCAAAAAGGGGUCAGUUG | |
| | AACCAUUGUAAGUUGGGGACCAUCUGCAGACAUGCUCAGGUGCUACUUUG | |
| | CCAGGCAGGGUCUGCUACUGUAACAUAAGACACCUCCCAGCCAUGGCUGA | |
| | UUGGACAGAGGUGGAACUCUGACCCACAGGUAGCAGAAUCCACUGUUUUA | |
| | CUAGGGACAUAGGAUGUGUGUGGCUUGACUCAAGCAGCUGAGGUGACUGA | |
| | AUCAGAUUCUGACUUUCAAAACAUUGAAGCAAGGAUGCAGAAACUGUUGC | |
| | UAGUCCAUGAAGGGCCCUGGGAUAGGGAGAUUAUCCAAAUUUGGGUGGCU | |
| | CAAGAGGUCAUUGGGCCAUGGGAAAGCUGAUUAGGAAGCUAUCUACAGAA | |
| | AGAGAAAAAUGGAACAAGUACAGAGAGAAGCAAAGAUGGAAUAUUAUGAA | |
| | ACCUCGGAGAGAAAUGGAGAGCAGCAGAUCCUCAGUUUCUGUUGCAUUUC | |
| | CAGUUCUUUAGGCCUCACGAGGCAGGUCUAUGUCCUGUCCUAGAUCCCAG | |
| | AGAUCUUUCUAUCCCAAAAUUAGGGGGGCCAUGUAAUUUAGAUCCAAAAU | |
| | GGCACUCUUUUGAAAGUGAAAGUGGCUGCUAUUAAUAAUUACAGACGAAA | |
| | ACCUCCAAAUCACUGGGAAAACUGGGUUGUAUGUGGUGGUGGGCUAGGAA | |
| | GUAUUUAACAACUGGGUCCCCAGGAAACAACAGCAACAACAAGCCAGAAU | |
| | UUGAUUGGUAGUGUUUGUCGAUUUCCAUGUUGUAAAUACUCUCACCAUAG | |
| | CUGAUUUCCAGCUACCAACUGGCUUGGAAAAUUCCUGAAAAUUUAAUAAU | |
| | CACCUUUCUCAAGAUGCGACAAGUCAGCUCCCGUACACCACUGGAUGCAU | |
| | ACUGCCUUACCUAAAGUACUCUCCUUCUAUACUAACCCCCUGCCCCCACC | |
| | UUUAUUUUUACUUGGGUUUUGUGAGUUUAUUUGAACCUUGCAACUCCAAA | |
| | AGCACCGAUUAGAACAGUAGUGCGUUUGGCCCUGCGACUGCAGGCAGUGG | |
| | GAGGCCUGCCUACAAGCCUGCCCUCCAGCUCCUUGAAGAGAGAGACUAUU | |
| | UUAAACUUCCCUUUCCUCCCCAGCAUGCCCAGCACAGCGAGGAGAAUGUC | |
| | GCAGCUGUCAGUAAAUGCCUACUGCAUUAAUGCUUGAGGUGUCCCUCAUU | |
| | GCACCCCUGCAGCAAGGUUGGCCAGCUGUGAUUUACAAUUUGCCUGUCAG | |
| | CAGUGGGAAAGUAACUUUGUGACAGCAGCAAACACCAGCUUCUCAGAGGA | |
| | UUUUGAGACCUGGGUUCUGACAUUUUUACCUAGAUGUGAUUGUUGUCAGC | |
| | UUAUCAUCCCCUGUCAAGUUCUAGGCUUCCUUGAGCAGAAAAUGAAGUUU | |
| | AUUAACUCAGUUAUGGGAUGCUCUGACUCUUUAGCCACAUGGGUGCUAAC | |
| | UGCUUGAUCAGUGCCUCUCCUUAGUGUGUUCACAUUGGCCUGAGGCGAGC | |
| | CCUGUCACACUCAUUCUUCCUUCUUCAAAUAACACAAUCAUUGCCUUCCA | |
| | AAAAGCAGAAUCAUCCAUCCUGUUUUAGAAACAAACUGGUUACCCAAACU | |
| | AAUUAGCUGAGCUUGGACUGUACAAAUACCUUUUUACCUGGAAUCCAGGU | |
| | UUUUCUUUACAUUUUCCUCUCUUUUAUCCCUAUUUAUGCUUGUAUGUAAA | |
| | AAUCUUUAUACAUUUGCAGAAACUGGUAAAUUAUCAGAAGAAUUUGGUUU | |
| | CUGUCAGCUUUCCUAAAAGCAAUUAUUUUCCCUGUUUCUGAGAAGUGAUA | |
| | AGAAUAAAAAAUAUUUCCUCUGAACUUUCUGAAUAUAUUAUUGUUAAAUG | |
| | AUUGAUAUAACACUUACACUGAGAAAAAAAUUUAUUCCAGGUCAGGCACA | |
| | UGGGUGAGCUGACACACACAUAUACUUUCUUCUCUAGCAGUUCUCGGGUG | |
| | AAAGAGAAUCAGGUGAAAUGAGAAUUCUAUGCUUGUACUAAUACUGUCUU | |
| | UUAAAGUUGUUUAUUUACAAAAGUCUUCCCAGGUUACAUGCCAAGCAAUA | |
| | AAACGGUUAAACAAUUAAAAGGUUUCCAAGACAACUUAUUGCAAAGUACC | |
| | AUAAAAAGUCAGCUAUGAUAUCACACUUGUAAUUGAAGACUCUAAGUCUC | |
| | AGCUUUCAGUAAGCAUGCAUUAGUAUCAAGAAGGUCAAGAUUCAUGGAUU | |
| | CUGGUAUUUUUGGUUAUGGCAAGAGAAAUUAACUUUUAUAAACCAUAGUG | |
| | UGAAAAUCAAUUCACCAGAAAACUCUGUGUGUUUACAAUGUGUCCAUUAA | |
| | UGUCCUGGCUAAUGUAAAAUGAUGAGGACAUGAGUCUAAGUAAUAGAUGU | |
| | UCAUAAAGUGGAAUAUAUAGAGAUGUUUCUAGAGCAUUAUUUAAAUUUUG | |
| | AAAAAGAAUAACAAUUCUACAUCUCCAGUAAGUGAGCAUGAUUUAAUUUG | |
| | GACAGAUAGUGAACUGCUAUCUGACCAUUUAAAACCAUGUUGUAGAAGAA | |
| | UAUUUGCUGCUGUGAAAAGAUAUUCAGAAUAUAUUGCUAAGUGGUGAAUU | |
| | CAGCUACAAAACACUAUAAUAACAAUUUUUGCAUAUUGUGUAUGUGCAUG | |
| | UGUGUGUGUGUAUUACAUAUAGGACUAGAAAAAUAUAAUAAUUUAACAGU | |
| | GGUUAUACCCAGUGGAUAUAUCUGGGGAGAGUGAUUAAGUCAUAUUGCUC | |
| | UAUUUGCCUAUUUGCAUUUUUCACUGUGCUGUUUUUACUGUUGAAGCACG | |
| | CUCGGCUGUUUUAGUUACAGGAGAACUUAUUCUCAUGAGUGUACUGAAUU | |
| | UUGAAGGGGAUAGAAUCUUACACAAUUAACUAAAAUGAGACAUAUAUAUG | |
| | UGCCAAAUGUAUGGUGUAGGGUCCUGUAAUUCAUUACAUUCCUGCCCUUU | |
| | UAUUUAGGGACAGGUUUUAGAAAGGUGAGUGGGGGUAACCUAGAGGAGUG | |
| | AAAACACCUUGGGUAACAUGAAUUGAUGCCUGCUUAAAGAGUUAAGGCAU | |
| | UUCCUCCCCCCCCCAUGGAAAAGGAGAACAAAGCUUUUACUGUACGAUUA | |
| | AUUAGAAUUCAGCUUUGCAUAGUCUGUGGUUACAAACUUCAGUGAAAUUG | |
| | GUGCAGGAUCCUAUUCUCAAAGGGUUACAAAGCACAUAUUGUUGUCAAAG | |
| | UACCCAGUACAGAAAGAUAGCAUCCUCAGAAUAAAAAUUAAGUACAUUUU | |
| | AUAUGAAGCGAUACACAUUAGACACUUUCAUAGGUUUAAAUAUUAAACAG | |
| | UGGUGAACAGUUCCAGGAAUCUUGUGAUUUUUUUUUUUUUCAUAAAAUGA | |
| | AUGCCCUUAUUUCCCAGAAAUCCAAGACUUAACAUUUCUUAACAGCUUAU | |
| | CUGAUGGGAACAAACCCAAACUGGCAUGUAAAUAUUAUCAACCAGGAGGG | |
| | UUUCCAAUUAAUAGAUCAAGGUAAAACAAAAAUUACAUUUUAUUCAACUU | |
| | UUGCCACUAGUGCUUAGCACAGUGCAUAGCGUAUUAUUGGCACUCAACAA | |
| | AAACAUGCAUAUUUUUCUUUAAGACUGUUGAAUAAACAGAACCAACGACA | |
| | AAAAUCACGUGAUUAUCUCAAUAGAUGCAGAAAAGGCCUGCAACAAAAUU | |
| | CAACACCCCUUCAUGCUAAAAGCUCUCAGUAAACUACGUAUUGAUGGAAC | |
| | AUAUCUCAAAAUAAUGAAAGCUAUUUAUGACAAACCCACAUCCAGUAUCA | |
| | UACUGAAUGGGUAAAAGCUACAAGCAUUCCCUUUUAAAACUAGCACAAGG | |
| | AAAGGAUGCCCUCUCUCACCACUCUUAUUCAACAUAGUAUUGGAAGUUCU | |
| | GGCCAGGGCAAUCAGGCAAGAGAAAGAAAUAAAGGGUAUUCAAAUAGGAA | |
| | GAGAGGAAGUCAAAUUGUCUCUGUCUGCAGAUGACAUGAUUGUAUAUUUA | |
| | GAAAACCCCAUCAUCUCAGGCCAAAAUCUCCUUAAGCUGAUAAGCAACUU | |
| | CAGCAAAGUCUCAGGAUAUAAAAUCAAUGUGCAAAAAUCACAAGCAUUCC | |
| | UAUACACCAAUAAUAGACAAACAGAGAGCCAAAUCAUGAGUGAACUCCCA | |
| | UUCACAAUUGCUACAAAGAGAAUAAAAUACAUAAGAAUACAACUUACAAG | |
| | GUAUGUGAAGGACCUCUUCAAGAAGAAUUACAAACCACUGCUCAAGGAAA | |
| | UAAGAGGGGACAAAAACAAAAGGAAAAACAUUCCGUCCUCAUGGAUAGAA | |
| | GAAUCAAUAUCAUGAAAAUGGCCAUACUGACCAAAGUAAUUUACAGAUUC | |
| | AAUGCUAUUCCCAUCAAGCUACCAGUGACUUUCCUCACAGAAUUAGAAAA | |
| | AAUUACUUUAAAUUUUACAUGGAACCAAAAAAGAGCUCGUAUAGCCAAGA | |
| | CAAUCCUAAGCAAAAAGAACAAAGCUGGAGGCAUCACGCUACCUGACUUC | |
| | AAACUAUACUACAAGGUUACAGUAACCAAAACAGCAUGAUACUGGUACCA | |
| | AAACAGAUAUAUAGACCAAUGGAAUAGAACAGAGGCCUCAGAAAUAAUGC | |
| | CAUAAUCCGAUCUUUGACAAAUCUGACAAAAACAAACGAUGGGGAAAAGA | |
| | UUCCCUAUUUAAUAAAUGGUGUUGGGAAAACAGGCUAGCCAUAUGGAGAA | |
| | AGCUGAAACUGGAUCCCUUCCUUAAACCUUAUAUAAAAAUUAACUCAAGA | |
| | UGGAUUAAAGACUUAAACAUAAGAUCUAAAACCAUAAAAACCCUAGAAGA | |
| | AAACCUAGGCAAUACCAUUCAGGACAUAGGCAUGGGCAAAGACUUCAUGA | |
| | CUAAAACACCAAAAGCAAUGGCAACAAAAGCCAAAAUUGACAAAUGGGAU | |
| | CUAAUUAAACUAAAGAGCUUCUGCACAGCAAAAGAAACUAUCAUCAGUGU | |
| | GAACAGGCAACCUACAGAAUGGGAGAAAAUUUUUGCAAUCUAUCCAUCUG | |
| | ACAAAAGGCUAAUAUCCAGAAUCUACAAAGAACUUAAACAAAUUUACAAG | |
| | AAAAAAAACAAUCCCAUCAAAAAGUAGGUGAAGGAUAUGAACAGACACUU | |
| | CUCAAAAGAAGACAUUUAUACAGCCAACACGCAUAUGAAAAAGAGCUCAU | |
| | CAUCACUGGUCAUUAGAGCAAUGCAAAUCAAAACCACAAUGAGAUACCAU | |
| | CUCACACCAGUUAGAAUGGCAAUCAUUAAAAAGUCAGGAAACAGCAAGUU | |
| | GCUGGAGAGGAUGUGGAGAAAUAGGAAUGCUUUUACACUGUUGGUGAGAG | |
| | UGUAAAUUAGUUCAACCAUUGUGGAAGACAGUGUAGCAAUUCCUCAAGGA | |
| | UCUAGAACUAGAAAUACCAUCUGAACCAGCAAUACCCAAAGGAUUAUAAA | |
| | UCAUUCUACUAUAAAGACACAUGCACACGUAUGUUUAUUGCAGCACUGUU | |
| | CACAAUAGCAAAGACUUGAAACCAACCCAAAUGCCCAUCAGUGAUAGAUU | |
| | GGAUAAAGAAAAUGUGGCUCAUAUACACCAUGGAAUACUACGCAGCCAUA | |
| | AAAAAGGAUGAGUUCAUGUCCUUUGCAGGUACAUGGAUGAAGCUGGAAAC | |
| | CAUCAUUCUCAGCAAACAAACACAGGAACAGAAAACCAAACACUGCAUGU | |
| | UCUCACUCAUAAGUGGGAGUUGAACGAUGAAAACACAUGGACACAGGGAG | |
| | GGGAACAUCACACACCAGGGCCUUUCGAGGGGUGAGGGGCUGGGGGAGAG | |
| | AUAACAUUAGGAGAAAUACUAAUGUAGAUAAUGGGUUGAUGGGUGCAACA | |
| | AACCACCAUGGCAUGUGUAUACCUAUGUAACAAACCUGCAUGUUCUGAAC | |
| | AUGUAUCCUAGAACUUAAAGUAUAAUUUAAAAAAAGAAAAAAAAUGCAUA | |
| | CUUGAUUGAGCUAGAAAAUGUGCUUGUCUGGAGGUCUCUAAUCUGUGUUG | |
| | CUAAUACAUAGAAUGACAUAAUAGAAAGACUAUGGACCGAGAAACCAAUA | |
| | CUCAGACCAGCUGCGAUUGAGUUCUAACGCCAGCUCUUCUGUUAACUCAG | |
| | UGUAUGGGUUCUUCUCCUUGUGGCUCCAUUUACCCAUUUGUUAAAAAGAG | |
| | GAUUGGACUAGAUUAACUCUAAUGAGAGUAUCUUUCAGAUCAGAAUACCU | |
| | AAGGGUUUCUUUUCUUUCUCUAUUGCCAAAGCUCAUGUAUUUAUUAAUGU | |
| | AUUCCACAGAUGCUUAUUGAAUGUCUUCUCUGUAUGUCAGGCAUUGUGCU | |
| | AUGUCAUGCAGAACAGGCCUGUAAACAAGGCACACACAGUCCCUGGCCUU | |
| | GGGGUCCCUUCCUCCAGUGAGAAAGUUGUGUUCCCUCCUACUGGGUUGAC | |
| | UAUAAUUUUUUUUUUAAUUCACUCAUUGUUUAAAGCAAGAUCUUAGAGGA | |
| | AUGGGAAGUUAUUCUUAUUGGGAGAAUUGCUUGUCAUCAUAGAGUUCUCU | |
| | UUGAUGAGAUAGAGAAAUUUAGAAAGAUUAAACUCCUAAAUUGCAUUUCC | |
| | UUGUGAAUUGUUGUAAUGGACCAGAAUAAACACAAAUUUGGUGUUUUCUA | |
| | GCAUUUUAGUGACAGUGUGGGUACACCUCACAUUACAAUGUAAAUCCAUU | |
| | UUAAAAAUGCAGUUAGUAACUUAUGUUUUAUUAACUCUCGUAUUUUUAGA | |
| | GUACCAGAGUAAACUGACCGUUUGAAGGAAUGCUUCAUAUAGUGAGUCAU | |
| | AUUGUCAUGUAAUUAUGACCCAACCUAUAUUUUUAUUUCAUUAAAGGACU | |
| | UUUCUUAAUGCAAAGCAUCUUUAAACUUCCAUGUAAAGGAGAUAAUUAUA | |
| | UUAAAAUAUUCAUAACCACAUUUUGACACAGCAUUGCUCGUUUUGUUCCU | |
| | UUGAUCUAAUAGUUUGUAGAAAAUAGAAUGUAACAAAUUAUCUCAUUUAA | |
| | UUUUACCUGAGAAACACCGUACUUUUUCAGGGUGUAUAAGUCAGGAUAAG | |
| | CUGGCUAUGAUGCUAUAACAAACAACUCCAAACUCUCAGAGGUUUAAAAC | |
| | ACUAUAGGUUUUCUUCUUGCUCAUAUGACACAUUCAUCUUGGGUAUGGAU | |
| | GAAUUGGACUGAUCACAAUUACUCUGGAACCCAGGCUGACAGCCGCCACC | |
| | AUCUCAAACAUUUUCAUGGUAAGUGCAAGAGAGCAAAAAGAGCUCUGAGA | |
| | GGGUCUCACUUUGGCAAAGAAUUGUGUGGUUAUAUCCAGUUAUAAAUCAC | |
| | UGGCUAGAAUUAGUCACAUGCUCCAUAUUUUCACAGAGGGGCAGGAAGUA | |
| | CUAUCCUACCAUCUGCCUAGGAAGAAGACAGCUGGGACACUUGAUGAAUA | |
| | AUGCUAAUUACUACCAAACACGGUAUGGAAGAGUUGUUUUCAGGCAUUAG | |
| | CUCCCUUAAUUUGCUAGAGAAUCAAUUAACACUUGGUUAGCUUAGCAAUG | |
| | UAUUUAGUUGAAAAAACUUUUUUCUGAAUAUUUCAAAGCACUAACUUUUU | |
| | UGAGAAGAUCUACUGCAUUGUACUACAUAUUGCUGGAAUUCUAGCAACAU | |
| | CUUCAAAUUUUAGAUUUGUGACAAUAUUUGUAAUGAUAUGUAUUUUAUAC | |
| | UUUUGCCCAUGGAUAUUAAUGACAUUUUUACUGUGAUUCUCAAAAGCAUU | |
| | AAAAAAGAAGAAAGGUUAGGUCAUAUAAAUGGUAUGAUUAGUUUUGUUUC | |
| | UUUAUUUCUUUUUCCCUUGGUGUUUUUUUAGACGCAGAGUUUUGCUUUGA | |
| | AUUGCUCUUCCAGUAUUAUUUUGAGAGUGCUCUACUAGUGCUUACUCAUU | |
| | UUCCAUUCUAAUAACAAUUCUAAGCCAGAACAAAUAGGAAUUUAUCAGAA | |
| | AAUUAUGGUCCACUCUCAACUACUUCUAAAUAUCAAUCUCAAAAGUUCAU | |
| | UUAAUAAUUCAACUUUCUUAGGUAUAGUAAUAAACAAUGUAUAAGUGCCU | |
| | UUUAAUAUACAUCUUAAAGUAUUAUUUUUAUAUUUAUUAAUACUUGUAUU | |
| | CAUUUAUAAAUUUACUUAUUUAACAAACUUUUAUUGUACUCAUGUAGCAG | |
| | GCAUUCAAACUGAUUUAGUGGUUUAAAAUAUAUUUAAUUGUAUACUAAGA | |
| | AUUUAUGAGAAUUUACAAAUAUUUUCUUUUUUUUUUUUUCUAAGUAAGUU | |
| | UGUUGUGCUUUAAAGGUUGUACGGUUCUAUUCUUCCAUAUGAAACUGACA | |
| | AUACAAACCUAAAAAUGGGAAAUACAUUUUGGACUACAUGUCCAGAGGCA | |
| | GGGGCUAGACUACCUUCUUUCCUAUGGCUUUUUGCAGAAGAAUGAUACAU | |
| | GUAUUUGGAAGAUAAAUGGCAGGGAGGUAUUAGUGUAAUAUAUUGCUGUC | |
| | AAAUAUUCUGGGAUUCAUACUUUAUUCACUUAACCAGCCAAAAGUGACUA | |
| | UGUGUUUACAGUGGGUCUUAUGAUUUGGUAAGGACUUAAAAUAUAAAGAU | |
| | UAAGACAUGAGCCAUAUUCUCAUGGAGUUCACACUCAACUGGGGGAAAUA | |
| | GAUAUGCCAAUAUACAAUUAACAAGUAUAUAAUGUAAUAAGUUCUAUGGU | |
| | AAAGGUAUCUACUGAGCACAAUGGAAAUACCAACCAACCUGGAGGCAUCA | |
| | GUGAGAUCUUCCAAGAAUGGAAGUGACAUUCGAUUAGGUCUUGGGGUGAA | |
| | AUACACUGGGGAAGAAAGGAAGGAGAACAUUGCAUCUAUAACAAGCAAUG | |
| | CAUGUAUAACAAGCAGCAUGAGUGACUAAAUGAGUGAACAUGUGAAAAAA | |
| | UGUGCACCUGGAGGACUCCAUACAUGAGGUGUGCGGUGCAGUAGCAGGAA | |
| | AUGAGGCCAAAGGAAAACCCGGGUCUGGACAGGACGGGAUCUUGGCUUGA | |
| | GUUUGCCUCAAAUAAGAAAGGAUAGGCUAAAGGAUAAGAGAGGAGAGGCU | |
| | AAAGAGCUUCUGGAGAGGCAUUAACUAAAGGGAUUGGAAUUUAUCUAGUA | |
| | GGCAUCUGGGAUCAUUGAAUUGUAUAAAGCAAGAGUGACAUGGUCAGGCU | |
| | GUUAUAUUCAGGAAAAUAAGUAAUUGUAGUGAGAGACAGAGAGGAAUAAA | |
| | UUAGAGACAGUGAAGCCUGGAGGCACAAAAACUUACAUCUUGGCUUCAUC | |
| | AGCUUAAUUUGCCAGUGACUAGACGCUAGGCAAUUGAGAUAGGUGACUAA | |
| | UUUUUGUACUUUAAUUUAUAAUAUUAUGACUAUGUGCUUAGAAAAUGACA | |
| | CCUGGUCCUUUAAUAACUUGAUUGCUUUUUCUAUAUAAAACAUCUCCAUU | |
| | AAAUUUUCUUUGAAGUACCAAUGCAUUUUCUAGAAAGCAGAAGACAUGUU | |
| | AAUAGCAGCGAUUGCCUUUCUCUGCUAUGAACAUCUAUGUGGUACUAAGU | |
| | AGUUAUUUAGGCAUCUCUAUUAAUAGACAGUAAUAGGUAUGCUGGGGAGA | |
| | UUUCUAAGGGAAUCAUUUCAUGAAGCACCUUAUAUGACUUUAAUAAAAAA | |
| | CCCAUUAAUGAAUAAAGGAAUAAGUUGAUCAAGUCACACAUUUGUAGCCA | |
| | CCAGCAUAGUUUGGAGAAUGAUAUGUAGUGGAAGUGAAAUGAAAAUGCUG | |
| | AGAGUGAUAUUUAUUCCAACUAUUUCCGAAGUAGAAUAUGAUAGUCCCUG | |
| | AAAAUAUUCAGAGAGGAUGGUGUUAUCAGCAGUAUGAGCUGUUUUUGAGA | |
| | AAUCGAGAAUGUAAAUGAGGGAGAAUGAUUCAUGCCAAAAUUGACUAACU | |
| | CCAAGUAAUCAUCUCUAGAAUGCUUAAGUAGGUAUUUUGCUAUAUUCCUG | |
| | UAGCUAUAACAAUAUAUCAUUUGAAAGAUUUUCAGAAAUGAGCAUCAUUA | |
| | UCAAUAAUGAAAAAAGUAGGUCAUAAUCUCUUUUUCCUUUGGACUUUUAA | |
| | AUGCCAAGUCUUGUAUCUUAUGAAUAUAAAAUCCAUUAUCUGUGUUACCA | |
| | AGAUAUAUCCAUCAUUAUUGCUUCUCUCUGUUUUGAAAUAACAUCAUUUU | |
| | UCCUAGGCAGACUAGGUAGAUUCUGGUAUAAAAAAUUUGGUAAUAUAUUA | |
| | UGACUGUGCAUUUAUUGGAUUUUAUGUUUAUUUUAAACUUAUAUGUUUUG | |
| | AUUUUUAAGUUUCUAAACAUUUAGGUCCCUUGCACUUUCGCAUAGAAGUG | |
| | UGUUUUAAUGCAUAUGCCUUGUGGUCAGAUAGCCAGUUGUUUUGGGAAUC | |
| | AGUUUGCCAUUUAGACACUCAGUCCCCCAAAAGGCCGGGAGAAUAUUCCU | |
| | UCAAAUGGCCCACUGGGCAAAUGUGUACACUUCUUUAUCUUGCCAUAUUU | |
| | UGGAGAGGAUAUUUUUUUGUCAAUUGGGAUUAUGCAUUUGUUAGAGCCUG | |
| | UGAGUUCUUGGGAGAUCCGUGUAUCUUAGCUUUUUGAGUUUUAGAAUUUU | |
| | UUCUCGGUAUAACAUUUUAAAGGUUAGAUUAUUUGGGAAGAGGUGUGAUA | |
| | AAGCUGAAUAUAUUCCUGUGGAAGAAACAAUUUGUAAUCUGGAUUAGCUU | |
| | UCUUUGACGGAAAGUAGUUUUGGCCAAAAGGUGACAAUAUUCUUCAACUG | |
| | GUAAGUGUGGAAUUGGAUGAGAGACACAAUCAAUCUGAAGGUGUAUGCUG | |
| | UUUACCAAAAAGUAGACUAGAAAUGGAGGAAAAUGAGGAAUUAAAUUCUA | |
| | UCUAAUAUACUCUGAUUCACUUAGUGUUUCUGCAUAACUAUUUCUGGACA | |
| | UUAUUUUCAAUUUUUUCCUUUAUUUAGAUGCAGUUCCACUGUAACUAGUC | |
| | UUACUACAAGCAAGCCUUUUCAGUAAAGAGAGGAGAAAAUAUACUCUACU | |
| | GUUUUUUAUUUCUUUGGAAUCAAAUGUUUGUUGAAUGCUACAUUGGGCCA | |
| | UGCAUAGAGUUGAACAUGACAAAUUGUUGCUAAAGAAAAUUCUAGACUGG | |
| | UGAAUAAUUUGUCUUGAAUGUUUUUUGAUUGCAUUUAGGUAAAGUUGUUG | |
| | GAGCAAAGUGUUCUGUUGGCUGCAAAUCUAUCCAUUCUGUUUGUUUCAGA | |
| | AAAACCCAGGGCUAGCCAGCUUUGAGCAUUAGUUAAUUGACAAUAUUAGG | |
| | AAAAAAAAUCAGCUCCAGAACCAUUUCCAUUGAUUGGGAAGACACACAGA | |
| | AUUAUUCCUUAAAGUGGUCCCAUUUCCAUAGAAGGAAACUGAAGGUAAAU | |
| | GAGUGACAGAAUUGCUUCAACCCACAGAUAAUUAUGAAUGAAUGUAGAUU | |
| | UACUAAUGUAGGUACUCAAACUCAGUGACUAUGUUUAUAUUGAUAUAUAA | |
| | AAUAUAUAUUUAUCUUUAUGAUUUUAUGUAUAAAAUAAUAUAUACUGUAU | |
| | UUUAUAGAGUAACUGAUUUUUAGAAGAGACAUUUUGGUUGGAGAAAGAUG | |
| | UUUAGAGUGAGACAGCUGGGUUUGAAUAAAACUUCAGUACUUAGUAUUUU | |
| | UCAGCUUUGAAUCUCAGAACAAGAGUUUCUUUGAAUGUACAAUGGGAGAU | |
| | GAAAGUACAUAUCAUGCAAAGUUGUUACUAUAUUUGCAAACUUGGACACA | |
| | AUAGACACCUAGGAAUUGGUAGUUAUUAUCCUUUUGCUACUAAUUUAUGU | |
| | UUAACAAAUAUUCCAGCGCUAUUGUGAUCAGAAGACUGCAGGAAGGUAUU | |
| | GUGGGAAAAACACUAAAAGGAACAACACAAGAUCUCUCUCUUUAAAGGAC | |
| | UUUGAUACAGUUUUUGAAGAGACAGACACAUUCAUACAUCCAAUAAAUCU | |
| | UAAAGAAUUCUAAUCUAAAGCAUACAAAACAAUGCAAAACCAUUAUAGGA | |
| | CAUAAAUGUAGCACUGGGUUUUUUUUUUUCUUUUUUCUCUUGAGACAGUC | |
| | UUGCCCUGUCACCCAAGCUGGAAAGCAAUGGCACGAUGCCGGCUCACUGU | |
| | AACGUCCACCUCCUGGGUUCAAACAAUUCCCCUGCCUCAGCCUCCCGAGU | |
| | AGCUGGGACUGCAGGUGCAUGCCACCAUGCCCAGCUAAUUUUUGGAUUUU | |
| | UAGUAGAGAUGGAGUUUUGCCAUGUUGAUCAGGCUGGUCUUGAGCUUCUG | |
| | ACCUCAGGUGGUCUGCCUGCCUCAGCCUCCCAAAGUGCUGGAAUUACAGG | |
| | UGUGAGCCACUAUACCUGGCCUGGAAUAAUUUUUAUGUCACCAAGAUGGA | |
| | AAUUUUAGAUCACGGUUGCCCUCUUUUCCAAUUUCUACUUAGAGAUUUCC | |
| | UCCUUACAUGUGGCGAUAAUAAAGUACUUUUAAUAAACUAAAAAAUGUUC | |
| | UUAAAAACCAUUAUCAAUAAUUAUGAUGUGAUCAAUGACAAUAAAUAGAC | |
| | UAUUUGUAAUUAUCAUGGAUUGCUUCAACCUGAUUCUAUAUUAAGGUCUC | |
| | AUUGUUAUUUCUAAUAAACUACUGAUUGCUAAAGCUGCAUUUUUAAGUUG | |
| | GCUCAUUAUAUAGGAAAUAUUCAGAUGUUAACAUAACUAUAGGUGCUCAU | |
| | AGUUGAUCACUCUACAAUGCUUGGGUGUUGCCAUUCCCUUCUCCCUGGGU | |
| | CAUGGCAGACAUUACCUAUUAAUCAAGGCAUCUUUGUUUCUAUUCAUUGC | |
| | AGUCUCACAAACCUUCUCAUGUCAGUGUGAUAUGGUUUAGAUUUGUGUCC | |
| | CUGCCCAAAUCUCAUAUUGAAUUGGAGGAGGGAUCUGGUGGGAGGUGAAU | |
| | GAAUCAUGGGGGUGGAAUUUCCCUUGGUGUUCUUGUGAUAGUGGGUGAAC | |
| | UCUCACAAGAUCUGAUGGUUUAAAGUGUGUGGCACUUCCCCCUUGCUCUC | |
| | UUUCUCUUCUGCUUCACCAUAGUAUGAUGCGCUUGCUUCCCUUUCACCUU | |
| | CCAUCAUGACUGUAAGUUUCCUGAGGCCUCCUAGCCAUGCUUCCUGUACA | |
| | UCCUGUGAACUGUCAGUCAAUUAUACCUCUUUUUUUCAUAAAUUACCCAG | |
| | UCUCAAGUAGUUGUUUAUAGCAGCAUAAGAACAGACUAAUACAGAAAUUU | |
| | GAUACUGGGGGAGUGGGGCACUCUUAUAAAGACAGCUGAAAAUGUGGAAG | |
| | CAACUUUUGUAACUGGGUAAUGCUCAAAGGUUGGAACAGUUUGGAGGACU | |
| | CAGAAUAAGACAGGAAGAUGUGGGAAAGUGUUGAACUUCCUAGAGACUUG | |
| | UUGAAUGGUUUUGACCAAAAUGCUGAUAGUGAUAUGGACAAUGAAGUCUA | |
| | GGCUGAGGUGAUCUUAGAUGGAAAUGAGAAACUUAUUCAGAACUGGAGUA | |
| | AAGUUGACUCUUGCUAUGUUUUAGCAAAGAGACUGGUGGCAUUUUGCACC | |
| | UGCCCUAGAGAUCUGUGGAACUUUGAACUUGAGAGAUGAUUUAGGCUAUC | |
| | UGGUGGAAGAAACUUCUAAGCAGCAAAGCAUUCAGGAUGUAACCUGGCUG | |
| | UUUCUAAAAGUGUAUGCUCAUAUGCAUGGGCAAAGAGAUUAUCUGAAACU | |
| | GGAACUUAUAUUUAAAAGGGAAGUAGAGUGUAAAAGUUCAGAAAAUUUGC | |
| | AGCCUGGCCAUGUGGUAGAAAAGAAAAACCCAUUUUCUGGGUAGAAAUUC | |
| | AAGCCUACUUCAGAAAUUUGCAUAAGGAAAGAGGAGCUGAAUGAUAAUAG | |
| | CCAAGACAAUGGGGAAAAUGUUUCCAGGGCAUUUCAGGGACCUUCACUGC | |
| | AGCUCCCAUUACAGGCCCAGAGGUGUAGGAGGGAAAAAUGGUUUUGUGGA | |
| | CCUGGCCCAGGGCCCUGUUGCUGUCUGUAGCCUUGGAACAAGGCACCUUG | |
| | CAUCCCAGACACUCCAGCUCCAGCUGUGGCUAAAAGGAGCCAGGGUACAG | |
| | CUCAGGCCAUUGCUUCAGGGGGAGUAAGCCCCAAGCCUUGGUGGCUUCCA | |
| | UCUGGUGUUGGGCUUGCAGGUGCACAGAAGACAGGAGUUGAGGAUUGGAA | |
| | ACCUCCACCUAGAUUUCAGAGGAUGUAUGAAAACAUCUGGAUGUCAAGGU | |
| | AGAAGUCUGCUUCAGGGGUGGAGCCCUCCUGGAGAACCUCUACUAGGGUA | |
| | GUGUGGAGGGGAACUGUGGGGUUGAAGCUCCCACACAGAGUCCCCACUGA | |
| | GACACUGCCCAGUGGAGCUGUGAGAAGAGGACCACUGUCCUCCAGACCCC | |
| | AGAAUGGUAGAUCCAUCGACAGCUUGCACCAUGUGCCUGGAAAAGCCUCA | |
| | GGUACUCAAUGCCAGCCUGUGAAAGCAGAUGUAGGGGCUGUAUGCAGUAA | |
| | AGCCACAGUGGCAGAGCUGCCCAAGGCCUUGGGAGCCCACCCCUUGAAUC | |
| | AGCAUGCCCUGGAUGAGAGACAUGGAGUCUAAGGACAUUAUUUUGCAGCU | |
| | UUAAGAUUUAAUGACCACCCUGCUGGAUUUCAGACUGGCACGGUGCCUGU | |
| | AGCCCCUUUGGUUUGGCUGCUUUCUUCCUUUUGGAAUGGGAGCAUUUACA | |
| | UAAUUCCUGUACCCUCAUUUUAUUUUGGAAGUAAUUAACUUGUGUUUGAU | |
| | UUUACAGGCUUAUAGGCAGAAGGGGCUUGCCUUGUCUCAGAUUAGACUUU | |
| | GGACUUGGACUUUUGAGUUAAUGCUGGAAUGAGUUAAGACUCUGUGGGAC | |
| | UGUUGGGGAGGCAUGAUUGGUUUUGAAAUGUGAGAAGGUCAUGAGAUUUG | |
| | GGAGGGUCCAGGGAUGGAAUGAUAUGGUUUGGGUUUGUGUCCCCAACCAA | |
| | AUCUCAUGUCAAAUUGGAGGCGGGACCUGGUGAGAGGUGAUUAGAUUAUG | |
| | GGGGCAGAUUUCCCCUUGGUGUUCUUGUGAUAGUGAGUGAGUUCUCAUAA | |
| | GAUCUGAUGAUUUAAAAGUGUGUGGCACUUCCUGCUUAUCUCUCUGUCUC | |
| | UCCUGCCCACUUGUGGUAAGACGUGCUUGCUUCCCAUUUACCUUCCAUCA | |
| | UGAUUUAAGUUUCCUGAGGCUUCCUAGCAAUGCUUCUUGUACAGCCUGCA | |
| | GAACUGUGAGUCAAUUAAAUCUCUUUUCUUCAUAAAUUACUCAGUCUUAG | |
| | GUAGUUCUUUAUAGCAGUGUGAGAAUGGACUAACAGAGUGUUUCAGGUAG | |
| | UCACAGCCAACUAGUUUGACUUGACACAUGUGAUAGGAUCUAUUUUUCAU | |
| | CCCAUCAUAAGCAUUUUUGAGUUUGAGAAAGUUCACACUCACUAUGUUAA | |
| | CUGAAGCUACCUUGUAGCCCUGUAAAUCUAUUUUUAGGAAACUUGAUCUA | |
| | CUAGUUAUAAUUCUCCCUUUUUCUUCUGGUCUCAUUAUAUGCUAGAAUUC | |
| | CUUGAUUAUCAACUCUGAAAACAUGGCAGAAUGCAGUGGUUCACAAAUAU | |
| | UGGUGUGCAUCAGAUUCACCUGAAGACUUGUUAAGGGAUUUCUGGGUCCC | |
| | ACUCCCCAGAGUUUCUGAUUCAGUAGGUAGCAAUAUUUUAAACAGGUUUA | |
| | GAAAGCUUUUAAGAGAUAUAAACUCAAGUGAUGCCUUUGGUGCCAAUCAU | |
| | GGAGCCACAUUUGGAGAACUACUGACAUAAUGAAAAGAACAUAGACUUAA | |
| | UUACAGAGAAAAAAGCAUUGAGAGCAUCAUUACUAGAUUCUUGAAACUCA | |
| | AUUGUUUUCAAUGAAGUUUUCCCAAUAAUAAAGCCUGUAUCAUGGAAUUA | |
| | CUUGAAGCAUUACCUGAGAAAUAUUUCAAAUAUUUAGCAGUAAUAACCCC | |
| | UCAGUGAAUGUACAUUCACUGUACUUUUUGUACUUUGUGCACUUUGUAAU | |
| | AAAAAAAUCAGAUCUAACAUUGUUCUAUGUACCAUAGGGAAAUGGAGAUG | |
| | GCGAUGUAUAGUUCUUGUUCUUUCUUCCAUGAACUGUUGUUACAUAUUCU | |
| | AAUCUUCAGAAACUCACUUUGUGUUAUCCUAUACAGCAUACCAUAUUUGC | |
| | UCUUUGCUCUGAAAAGCUUGCUUUAGGCACUAAGGAGAAAGUUUUACUUU | |
| | UUUACUUUGAGUUUGGAGAAGUGCACAAAUGGUAAUCCAGCAGGUAACUG | |
| | UGUAUAAAAGCUGUGGAGCCACAUUAGGCUGGGAUUUCCCUCUUUACAGG | |
| | UAAUAUUUUUAAAUGAAUGUGAUAAUACUUAGAGCAUAUUUUACAUCCUG | |
| | CAAAGGAGUAUACUAUUCAAAUAUUUGUGCCAGAUUGCGUUGUAAAUGCA | |
| | GCACCAUUUAAUUUUAAUUUAAUUUAAUUUUUAAUUUUUUUUUUUUUUUU | |
| | UUUGAGAUGGGGUUUUGCUCUUGUUGCCCAGGCUGGAGUGCAAUGGUGCG | |
| | GUCUCAGCUCACUGCAACGUCCGCCUCCCAGGUUCAAGUGAUUCUCCUGC | |
| | CUCAGCCUCCAGAGUAGUUGGGAUUACAGGCGCCCACCAGCACACCCGGG | |
| | UAAUUUUUGUAUUUUUAGUAGAGAUGGGGUUUCACCAUGUUUGCCAGGCU | |
| | GGUCUCGAACUCCUGAUCUCAGGUGAUCCACCCGCCUUGGCCUCCUAAAG | |
| | UACUGGGAUUACCAGUAUGAGACACUGCUCCUGGGUGCACUACCAUUUAA | |
| | UUUUAAAUUUUCACUUAUUAGUUCACUUGAAUUGCCCUGAAGAAUACUUG | |
| | GUCCGAACAUUUACAUCUAUGGAUAAGGCACUGAGAAACAGAAAGCCUGA | |
| | AGGAAUUUCUCAAGGAUGAUGAAGAACCUGGCUUAGGUUUGAUCUCAAAA | |
| | CACAGACUCCUUAUAAAUAAUUCACUCACUUAGGUCAUUCUCCUCCAUGA | |
| | UUGCUCUUCAGUUUACCAGAGGUUUAAUUUGUUUCCCCAAGUAUUUUCAC | |
| | AUGCAGAUUCUUUAUAUAUUACUGUAUUUUUGUGCUUAGUAAUCUUAAUA | |
| | AAAAUAUCAAGAGUACUGUUUAGGAAAUAUGUUAUCUAUGGCAAAAUAGU | |
| | UCUUAAUUAUAAUGUAGUGUUUUUGGACAGUAUAAAUGAUAGCUUAGAAU | |
| | UGAAAAGAGAAUUACUAUGCUUACCAUAAAUCUCCUUGACAACAAGUUCA | |
| | AUGUUUUGAAUACUGAAUCAUUUAGAAUUUUAUGCAGAUUUAUAGGUCUC | |
| | UCACCUUGGCAUCUCAUUCCUUUUUUAUACAUCUGGGAUUUUAACAGAUG | |
| | UAUAAAAUCUGUUAUAAUUAUUACUGCUUUAUUUAUCCCCAUAAUUGUUA | |
| | CUGCUUUAACAAAUAUUUUUGGCCGUCCAACUCAUUUGUAUUUGAAAUGU | |
| | UUGCCUAGUUUUUACUAUCAUCAUCAUCAUCCAUCAUCUUCAUCUUUCUU | |
| | CAAUAUUUAUUGAGCUUUGAAAAACUGUGAAAAUCAACUUCAAAGAACAU | |
| | CUUGAUUACUUUACUGAAAUGAAGGUCCUUAUUGGAACCCUCACUCCAGA | |
| | AAAAAAGAAGUGAUUCACAGAUUCUGGGUCUUCUUUCUAAGGGAUAUAUA | |
| | AUGUCAGUUAGGCAGUUACCAUCCCCCAAACUGCUUGCUUCUCAUUUUGU | |
| | AAUGAAAAGAAAGCUCUCUCAGCAUAUAGUAUUCUGUACAGAUUGCCUUC | |
| | CUGCCUUUGGCGGAGCUCCUGUUUCUCUGUGAAUCAUUCCUUGGUACACA | |
| | GUGUUACCAUUGUGUUAUCUGAAAGAGCGAAUCCUGCUGGGUGGUGGUGC | |
| | CGCUACUCCAUUGGGAAGGUCACAAGUUCAUGUCUGCUUUGCCUUCUGUC | |
| | UAGUGAAAUGUGUUCCCACUAAGGAAUAGAGAAUUGCAAUAUUUUUCUGC | |
| | UUAUGGAAGGUCAGGCGUUCAGUGGUACCCUAGCAGUGUUGGCUAAAAGG | |
| | CCAUCAAUAGUGGCAAAUUCAAGGCAAGGCUUAAUCUUGCCUGUUGUCUA | |
| | UCAUUCCACAUGUUUUUUUACACCUCUUCUCAGUGGCUGAAAAAUGAAUU | |
| | UGCUUAAGGACUUGGUUGAAGUGACUGAGGAUGAAGCAGGAGGCCCUUAA | |
| | UGGGACCCUGGAGUGAAAGCGCUUGCAGUUUAACUAAGUGGUCUUUAUUG | |
| | AUUAAGCAGGCAUUUAUUGAACAUCAGCUCUAUGCCAGACACUGCUUUAG | |
| | GUGCUGGAGUUACUGAGGUAAAUGAAACACCAAAGGUCUCUGUUGUUUCA | |
| | GGGCUUUUAUUCUAGUGGAGGUGUCAGGCAAUAGCAAUGGCAGAGAGUGA | |
| | GUGACAGAAGCUGUUAGGAGAUUAAAGCUGAGGAAGGUACGAGAAAGGGC | |
| | AGGGCUGUUGUUUUACAUCAGGUCAUUAGGGACAGACUUUCUGAGGAGAA | |
| | GACGUAUGAACAGAAGAGAACCAUAGAAAGAUCUAGAGAGAGAGCUUUCC | |
| | AGAAAGAGGUAAAAGCAAACAGAUCCUGAAGUGAGUGUGAGUCGCCAUGA | |
| | GACGGGUGCCACAAAUUCAACAUUUGUUUGAAAUAAGAAUAUUUUGUUGA | |
| | UAAUGAUUAUACACAUCUAUAUAAAAUACAUUUGAAAACACGACAUUAUG | |
| | CACAAAAAACCAUCACCACUUUUAAAAAUAUGUGUAUAUAAUGAAGGUAU | |
| | AAAAUAAUAUUUUCUGUCAUAAAUUUUUCCCCAGUAGAGCCAUCAUAGGG | |
| | UUAAAACAAUAUGUUUUAUACAUAAUGUACUGUAACUGCUUUUUUAACAA | |
| | CUGAAAAUGUACAGAAAAAUGGUGUUUUGACUGUCUAUCUUUAGUCUUCA | |
| | GCUGUUGCUGUCACGAUUCAGAAAUUUGGAUCUCUGCCUUAAGAAGCACA | |
| | UUUUCUUAGCUAUAAAAAAAUCUGAUGGAAUCUACAACUGUAAUCAUAGG | |
| | CUGUGACUUUAGAAUAUAAAUCACUUGUUAUAAAAGGGGGGUGUUGGUGU | |
| | UUCUUUUCUUUUCAUUUGAUUCUCAUUUGUGUUCUCAAUAUGCUUUACUU | |
| | UGAGUUUGUUCGUAUUUGGAUGAAGAUAAAAUUUUCCUUGGCUUCAGUGU | |
| | GUGGCAAGCAGUGCCCAUUUAUUUUAAUAGAUACUGUACCAACUAAAUCC | |
| | CAAUAUGUGUCAGGAAAUUCAGAAAACUGAACCCAGAAAUGAUGCUAGAC | |
| | ACAGAUAGAAAGGAGGACUUCAAAAAUUCCUACAUGUUACUUUGGUGAUU | |
| | GGUGUGAAGGAGUACUUUAUAGUAUCUUCUAAAAGUACAGUUACUUUCAG | |
| | AAAAAUGAGAAAAAUUUAAAAAGAUUAAAAAACACAGUAUCAUUAGAAUU | |
| | UGGUCUCUUGCCUCUAAAUUUUCUAAAUUACCAAAAAUAAAAUAAAAGAA | |
| | AUUUAUUGGCUAUAGAUAAUAUAGCACAAAUUGGAUAAUUUUAAAUCUCA | |
| | GACAUGUAAAAUCAGUACAGAUUUCACACCAAAACACUAUUUGUAGCAAU | |
| | AGUAGCCAUGAGAAUUAAGUAUCUGACCUGGGAGGCCCCAAAAUAUCAAU | |
| | CUCAGGUGUGUCCGUUAGAAGGUAAAAAAUCUGGAAAUAGAAUCCUGAGA | |
| | UGAUAUUUCUACCUAUAAUACUUAAGGUAAAGAAAAGGAGGUACAUAAGA | |
| | AACGUUUCUAGAUUUUAACACAAGAGACAAAUAAAUUCUUUUUGACAUGA | |
| | AAGACAAAUAAAUUUUGAGUUGCAAAUGAGUGAACAUGCCAAGGUACGUU | |
| | AAACUAAUAUACUUUCUUCCAAAACUGGCUUAAGAUUGGUAAAAGUGCAU | |
| | UCAGGCGAAAUUAUUUUUAAAAAUUACUCAUUAAUAAAAUUUUGCUAAUU | |
| | UUUGUUGCUUAUUCAAAUUGCUUUGUGUUGUCCUGAAAUAUGAAAUUAUG | |
| | GAGAAACAAUUCUAGUUUCAGGAUACAACUUAAUUGGAGUUAAUAGGUUU | |
| | UUAAUACAGUGUGUUGUAUUUAUGUGUAUAUAAAUAUGGCGUAUUCAUGU | |
| | UUCCUUUUUUUUUUUUUUGCAAUAAAGUAUAGUGGAUUAAAGCCUAAGUU | |
| | UUGAAGUCAGAUGUUUUUGGGUUCCAAUACUUACCAACUGGUUGACCUUG | |
| | GGUAAUUCAUCUAAUGUUUCUGUAAUUCAGUUUCUUUAUCCGUAAAGUGU | |
| | AGAUUAUACAGUUCAAAGGGGGUAUUAUAAGGUUAAAUGUGAUAAUGUGA | |
| | UGAUAAUGUAUAUAAAAUUAACUAGUACAUAGCAAACUCUCAAUAAAUAA | |
| | UGGUUAUUAUCUCUUAGUUCCCAGUGGUUCUCUCUUUCUCUUUCUGCCUU | |
| | CCCCCCAAGAAAGGGCUGGAACUGAUUCUUUUCUCAUAGCCCUUAGAAAG | |
| | AACUAACUCAGCUGAUGCCUUCAUUUUUGGACGUCUAGCUCCCAGAACUG | |
| | UGAGACAAUAAAUUUCUGUUGAUUAAGCCACCCAGACUGUGGUAUUACCA | |
| | AGAAGUACUAAGGUAGUGCUGUAAAAAUACUUUAAAAUGUGGAAUUUGGA | |
| | AUUUGGUUGGCAGAGACUGGAAGAGAUUUGAAGUGCUUGGUAGGAAAGGU | |
| | CUAGAUUGCCUCCAGGAGACUAUUGUUAGACUUAUGAAUGUUAAAGAUGU | |
| | UUCUGAUGAAGUUUCAGACAGAAAUAAGGAACAUCUUCUUGGAAACUGGA | |
| | GGAACGGUGAUCCUUAUUAUAAAAUAGAAAAGAACGUGGCCUACUUGUGU | |
| | UCUAGUUUCUUUGUGAAAAAUAGAACUUAUAGGUGAUGAACUUGGAUAUU | |
| | UAGCUGAGGAGCUUUCUAAGCAAAGGGUCGCAAACACAUCCUGGUUUCUC | |
| | CUUACUGUUUAUAGUAAACUGUGAAGGGACAGACUACAUUGAAGAAGGAG | |
| | UUGUUAAGCAAAACGUAACCAGAGUGUGAAGACUGGAAAAAUUCUCAGCC | |
| | UAUCUACAUUGCAAGAAAUGAGAAAACGUGUUCUGGAGAGAGCAUCAAAA | |
| | GUGUGACUAGACAAUCAUUUGCUACAAAGAGAAGCAUGUGACUCAUUAAU | |
| | CCAAUCAACCAUCUCAGCAAAAGACAGGAAUAGAUAUGUGAUCAUCCAGG | |
| | AAACAUCUGUGAAAGACCCUCUUGUCUGGUGGUUUGGAACCCUGUGAAUU | |
| | GCACAGGAGGCCAACAAGAUUUUUGAGAGUUUUCUAUCAGCAGAAACACU | |
| | ACAAACUCUGACUGAAGGUGACAGAACUGGGAUGAAGUGAAGGAAAAUGG | |
| | UCAGACUUCUGGGAUUCUACAAGCAGGAAAUGGGUUGAUUGAACUAUCUG | |
| | GCUGGUUGAUAAUGCAGAUAGUUUUUAAGAAAAGAGAAGAAUGAUCCAGA | |
| | AGGUAGUUUAGAGGCCAGCAGAGCUGCUUCUGUCAUCAUUGGUCCAGAGA | |
| | GCACAAGCCUGGCAACACUGGGGCCAUCGCCUCAUUGGUUCCAGAAAAAU | |
| | AGGACCACCUCCUUGGUUUCAGAGGGCAGGUCUGGCCACUAAUGCUCAGG | |
| | UUCUAGAAAACUGGUGCCACCCCAUUCAUGGGCCCAAAGGACAGAACACU | |
| | GAGCCAAAGAGGAUUAUUCUCAAGCCUCAACAUCUAAUGGAAUUUUUCGU | |
| | GCUAGGUUUUGGACUUAUGAUCUCUUUCUUCCUUUUGGAAUAGGAAUGUU | |
| | UGUCCUGUGCCUGUCCCACCAUUGUAUUUUCAAAUCAGAUAACUCAUUGU | |
| | CUGGAUUUAUAGGUUUCCAGAUGGAGAAGAACUUUGCCGCAGGAUGAAUC | |
| | AUACUUCAAGUUUCACCCAUAAUCGAUUUGGAUGAUUUAAAUGAGAUUUG | |
| | AGGCUCAGAGUUGAUGCUGGAACAAUUAAGACUUUUGGAGGUGUUGUGAU | |
| | GGGGUGAAUGUAUUUUGCAUAUGACAGACAACGACAUGAAUUUUUGGGGU | |
| | CAGAGGAUGGACGUUUAUGGGUUUGUGUCCUCCCCAAAUCAUAUGUUGAA | |
| | AUUCUAACCCCCAGUACCUCAGAAAGUGACCUAUUUUGGAGAAAAGAUCU | |
| | UUACAGAGAUUAUAAAGUUAAAAUAAGGUCAUUAAGGUGGGUCCUAAGCC | |
| | AACAUGACUGGUAUCUUUAUUAAAAACGGUAAUUGCAACAUUGAGACAGC | |
| | AUAAAGGGAAAGCCAUGUGAAGAGGCAUAGGGAGAAGGCAGCCAUCUAUA | |
| | AGCCACGGAGAAAGGCCUGCAACAUAUUCUUCCCUCACAACCCUCAGAGG | |
| | AGCCGAUUCUGCUGACACCUUGAUUUUGGAAGUCUAGCUUCCAGAACAGU | |
| | GAGACAAUACAUUUCUGUUAUUUAAACCACCCACUCAUUUGUACUUUGUU | |
| | AUGGAAGCCCUAAUAACGUAAUAUAUACUAUUGUGUAUGUCAGAGGUUCA | |
| | GACUGUAUUAUCAAAAAGGAUCUAAUUUAUAUUGUAUAAGGAUAGUUGAA | |
| | CGAGUAUAGUUAUUUAGGAUGGAAGAGAAAAGUAUUAAAGAAAAUAUCUU | |
| | UGCAGGGCUAUAUAUGACCCAGGAAUCCUACAUUUGGGUAUUUUCCAUAG | |
| | AGGAAUGACAGCUUAUAUUCACACAAAAACCUGUACACGGAUGUUUACAG | |
| | AAGCUCUAUUUAUAAUUGAGCUCAAAACUAGAAACAGCCCAAUUGUCCUU | |
| | CAGUGGGCAAAUAAGUAAACAAACUGGUAGAGACUUACAAUGGAAUGCUA | |
| | UUUGGCAAUAAAAAUUAAUGAACUAUUAAUAAAUGUACAUCUUAGCUAAA | |
| | CUUCAAGGACAUUGUGCUGAAUAAAAACUUGAUCUUAGCAGUUUAAACGC | |
| | UAUAUGAUUCCAUUUAUAUGAUAUGAUUGAAAAGACAAAUCAGUGAUGGA | |
| | GAACAGAUCACAGAUUGCCAGGGAUUAGUGGUGGCAGGAAGGAUGUGACU | |
| | AUAAAGGUGUAGCAGAGAAAGUUUUCUGAGGGUGACACAACUAUUCUAUA | |
| | UCCUGAACAUAUUGGUAAUUGCAAGAAUAUCUAUGUGUGUCAACUUUCAU | |
| | AGAACCCUACACCAAAAAAGGUGAAUUUUAUUUCGUGAUAAAUUAAAAAG | |
| | UAAAACAAAAGAGACAAAAAUAGUAACCAGGGAAGCUUUCAAAAAAUAUA | |
| | UGUUCUUGUUCAAGACCAGCAUGGCCAACAUGGCGAAAUCCCAUCUCUAA | |
| | UAAAAAUAGAAAAAUUAGCCAGGCGUGGUGGUGGGCGCAUGUAGUCCCAG | |
| | CUAUUCAGACGGCUGAGGCAGGAUAAUUGCUUAAACCUGAGAGGUGGAGG | |
| | UUGCAGUGAGCCGAGAUGGUGCCACUGCACUUCCAGCCUGGGUGACAGAG | |
| | UGAGAUUCCAUCUCAAAAACAAACAAACAAACAAAAUAUAUAUAUGUUAU | |
| | CUUUAAAAGUCUUUGCUAAAACUCUCAGCAUGCUUACAUAUCAAAUGUCU | |
| | CAAGCCUUUGAUUUUACAUAUGUAAUAUUGCAGUGUUUUGAUUAUAUGAA | |
| | AUAUUAUAUUGAAGUAAAUUUGAUUAACUCUAAAUUUAAUUUUAAAUUUA | |
| | AAUUUAAAUUCUAGGACUUUAUAGCAAGUCUUGACAAAUAAAAUUCUAUU | |
| | UUCUUCGUUAUGAACUAAUGAGCCAUCAUCAUUAAUGUUACUGAAAUUUU | |
| | UAAAAAUUCUAAUAUGAACAGAUUUAAAAUAAACAGACUAUGUAGUGAAA | |
| | AACAAAUCUUGAAAUUAAGUCAGCUGAUCUUGAUCUAACCUACCAUUUUG | |
| | GAAGAAUUUUCUACAUUGAUAUGUAUUAUGUACCCUUAUAAGGCUCAUAG | |
| | UAGUAAAAUAUUAAUUUAGGAAAAUAUUGUCUCUGUGAAAUAGCUUGUGA | |
| | AUGUCCACUUAAACACAUAAUGGAUCUCUGCAUAUUGUUCUUUUUUUAAG | |
| | AGCAGUGGAGUGGAUUUGAUUUAAUAUUAAUGUGUGCUAUACUCUAUGAU | |
| | CUCUGUAGUAUCACAGUUAACUGGACUUAUAUAGGCAAUUAUUUACAUUU | |
| | AAAAUUUUACAGUGUAGAUUAAUACUAUGUUAUUUUUUUCUUAUCUAUUU | |
| | CAGACAAGACUACUGACAAAGUAGGAAAUAAUAAAAAGAUAAAAGCUUAU | |
| | UUUAGAGAAAGAGGUUUUCCCAAAUAUAACUUUGUUUCUAACAGCUAUGU | |
| | CCUUAAUAUUUUAUUCAUUUGAAAUCACCCUUAUAGACAAUUUGCUGACA | |
| | GGUUAAUAUUAUCUUUGGGAACUGGAGUACCCUCUUUUAGUGUGGUUUUU | |
| | ACAACAUCUACCAUGGGAACAGCUUCCCCUUCCAUUUAGGUAUCUGACUC | |
| | AUCCUUGCUAAUAUAUCUUGAUUUAAUGAUGACCUAGGGAAGAAAGAGAG | |
| | GAACUCCCUUAGUAUUUUUAUUCUAUGUAAUCUGCCUUCUCAGAAUGUGC | |
| | CUUGUGAAAGAAGCACCUAAUUACUCUGCUAAAACCAGAAAGCACUGUGA | |
| | GGAGAAAGCCUGGCAAGUUUCUCCUACCAGUCUGCCACCUAAUGAAUAGA | |
| | AAUUGCAACACACUGGUAUUCUGCUUUUGCACUGCGGCAGGAAUGUGAAU | |
| | AAUAAUAAUUAUUAUUAUUAAAAGAACAAAGAAAUGUUAGCUAAAAUGGU | |
| | AAACAUAGAUAAACUGCUGUGAUUUAAGAAGUUUCCAUUUGGAAUCCUCU | |
| | GUAAGCUCAAGUAUAAAAAGGAUAUUGGCUGUAAUCACUUUCAAGACAGU | |
| | AACAAAAAUGCAGGGCUAAUAGCAGGGCAUGAUUUGACGUGCCAAAUUUA | |
| | UUUUUCUUUCAUAUAUUUAACUGCUAAAUAUUAAUCAAUCAGAAUACACA | |
| | AUGUCAGCCUCUGAAGAUACAAAGGUGAAUACGAUAGUUUAUGUCCUUGG | |
| | AGAGCUCAUAGAUUAGGCCAGGAGACAAAUGAAAAAAAGUACCAUAACAU | |
| | AUCACAAAACAUUCUGAGGGAGACAGCCAAUGCUGGUGGAGUACAGAUGG | |
| | AUGAAACAGCUGACUCUUCUUAUGGAAGUUGAGAAAAAAUUUACUGAAGA | |
| | GUUUUCCUUUGAAUUUAGCCUUAAUGGAUGAGACAUGACAUGCUUGACAA | |
| | AGAUGAUAGAGAAAUAACACUUCAGACAGUGAAAACUGCACAAAAUAAUA | |
| | AAUAUACAACAUUUUGAAAAUGCUACUGCUUAAUGCCAAUAUAGCUCUUA | |
| | CUCUAAUUUGAGAGAAAUAGCUUAUUCCAUUUUGUUUGAUCCCUCCUCCU | |
| | UUGUCUUAUUCUGCACCAAGGCGAUCAGUUAUUUAUUCAUUCAAUGAAUA | |
| | UUUGUUUAAAUACCUACUAUGUAGCAAACAGUAUUUGGGGAGCUGAGAUU | |
| | ACAGGAUCGAACAAACAAAGCUCUGUUCCUUAUGAAAGUUAAAUUUCAGG | |
| | AGGAGUAAACAGAUGAUAACAAAGUCGUUAAGUAAUAUCGACUAUGAUAA | |
| | ACAAGUAAUUAAUAUUAUACGCUGACAAGUACUAUGGAGAAAAAUGAAUC | |
| | AGAAUAGGGGAGGUCGGUAGGUUUAAACAGGGUGAUUAUGGGGGACUUCA | |
| | UGAAGCAAGCGAUAUUUGGGCAGAUGCUGAAGGAGAUGAGGGAACAAACC | |
| | AUGCAGAUAUACUGGGAAAAAGGAAACUUGAACAACAUUUUCUGUGAACA | |
| | AAAAGGAGGCCAGUGUGGCUGAAGAAGAACUAGGGGGAGAGUUGAAGAAG | |
| | ACGAAAUAAGAGAAGUACUGCAGUUGGAGAUGGGGAAACCUCAUGUAGGG | |
| | ACCUCAAGGCCACUGUGUAGACUUUGCUUUUACUCUAGGUGAUGAGGUGU | |
| | CAUUGGAGGGCUUUGAGUAAGCAAUUCUUUCUUGAAUGGGACCAUCUCUA | |
| | AGAAACAUGAAACCAUGCUCAAGCAUCUAGGAUACAAGCCUCCCACCUAG | |
| | GAUUCAGGUGACUCUAUCAGGUAGUAUGGGCUAAAACUGAUCUUUCUGAA | |
| | GCACCAUUUUCCUAAAGAUGAGUAAUUAAAUAGCAUCAUAUGGAUAAAAG | |
| | UGAGGUAUUCACAUCAUCUUAUAAAGAUAUCAUGCUAGAUAACUUCAGAC | |
| | UCAAGGAUGUUAAGUAUAUGUGAAGAUGAACUUCUAGCAAUAUGACUGGA | |
| | UCACCGUGGGAGCAUGAAUUCCGUCUUCUGUCCCAUAAGGUAUCCUCUGU | |
| | UAUAAAAAGCUUGGAUAGCAUUGGUGAAGUAUAAUAAAAUGGACACUAUA | |
| | GUUAGAGUGGAAAGACCUGGUUUUGAGUAUUGGAUCAACUGCUUAAAAGC | |
| | UAUGCAAUUUUAUUUUUUGAUUUAUUUAUUUUUUUUUGAGAUGGAGUCUC | |
| | ACUGUUGUCUGCCUGGGCUGGAGUGCAAUGGCACAAUCUCUGCUCACCAC | |
| | AACCUCCACUUCCUGGGUUCCAGUAAUUCUCCUGCCUCAGCCUCCCAAGU | |
| | AGCUGGGAUUACAGGCAUGCGCUGCCACGCCCAGCUAAUUUUUGUAUUUU | |
| | UUAGUAGAGGCGGGGUUUCACCAUGUUGGCCAGGCUAGUCCUGAACCCCC | |
| | CACAUCAGGUGAUCCGCCCGCAUUGGCCUCCCAAAGUGCUGGAAUUACAG | |
| | GUGUGAGCAACUGUACCCGGACUUGCGUCUUGAUAUUCUUAUCUGAAAAU | |
| | GGGUAUAAUAAUUUACAAAUAAUGUACAUUUGGUCUACAAAUAUUUAUUG | |
| | ACUGUCUACUCUGUGCCAUAGUCUAUCCCAAAUUCCAGAAAUACAGUAUU | |
| | GAACAAGACUCAGCCCAGCCCAAAAGGAUUUUAUAUUUCAAACAGGGAAU | |
| | UUCAACAGUAUUUGUCUGAUAUAUUUAUGAGGAUCAAAUAAAGUAAUAUA | |
| | UUAGAAAUGCUUACUGCCAUAUAUGGCACUCAGUAGGAAUGCAAAAAAUG | |
| | UAAGUUACAUCUCUAUAAUUCUUUUAAAGAGUUGCCAGUUAAUUUUCUAA | |
| | GAGAGCUAGAUAACCAUUUUUAUUCAGUUGAAUAUUAUCUUCUUUUCAAU | |
| | CAUGUUAGAAUAAUUCUCUAGCUUCGUUUUUUAAAACAAAAUAUAAAAAU | |
| | UGGGAGAAGUUGCAAUUUGGGGGUUGCUGUAGUUAUAUAUAACAGUAUAU | |
| | UUACUGUUAUAUACUGUUGUAUAACAGCAUACACUGCUGGAAGAGGUAUG | |
| | UUAGUUCAUUGGUGCUCUGUUACCUUGUCUUUGACUGAAGGAAGGGAUCU | |
| | UGUAUAGCUUUACUUGAAUAGAGUCUAAUGGUUGUGUGUUGUUUUAGAAA | |
| | AAAGGAACCAGCUAGUGAAAUAAAUAAAGACAUGAUCAAUAAUAAUUCAA | |
| | UUUUAUUAAUAUUGAUAGUCUUAAAUCUGUUCCUCUUUUAAAGAUUUUUU | |
| | UUUAAAAGCUUCUGGAUUGUCUUUUUAAAAAAAGCUUCUUAAUUCAUUGU | |
| | CUUCUCAAAUAUCCAGCUUUGUAUUUACAAUAAGUGGGAACCAUGUUAUU | |
| | UUGAGUGCCAUGUUGUGCUAGGUAUAAUAUUAUUGUAUACAAACUGCAGU | |
| | UUUGUCCAUUUAAAAUACUUUUGUUUUAUAAUAGUUGAAAAGUAGUUACU | |
| | CGUAGAAAAUUUAUUCCAGGUCAACUUUUAAAGUUUGAAGGAUAUUGAAU | |
| | AUAAAAAGUAUGUAAUUAUUUUCAUCCAAAAUAAUUGUAUUACAAAGAUG | |
| | UUGAAUUUUAAAAACAUUUUAAACCAAAGUUAUCAUAGUGUCACUAUAAA | |
| | UUUAUAAUAUUUUUGGCUGUAAUUAAUUGACCUUUCUUUUCAAUUCAAUG | |
| | AAAAUCAAAAUGUUUCCAUUUUUCCACAAUAUUUAUAAUAAGUAAUGCAU | |
| | UAUUUAAUUGUUAUUGACUAUAUAACUAUUCAAAUCCUUUAAUGAUAUUU | |
| | UAUUAUGAUAAAUUAUUGAUUCUUAUUGCUUUGACAGGAUGUGAAUAUCA | |
| | GAAAUUUCAUUAUUUAAAACUGAGACAGGCUGGGCACGGUGGCUUGCACC | |
| | UGUAAUCUCAGUACUUUGGGAGGCUGAGGCAGGCAGAUUACAUGAGGCCA | |
| | GGAGUUUGAGACCAGCCUGGCCAACAUCGUGAAACCUGUCUCUACUAAGA | |
| | AUACAAAAAAUUAGCCAGGCAUGGUGGUGGGCGCCUGUAAUCUCAGCUAC | |
| | UCAGGAGGUAGGAUCGCUUGAACCCGGAGGCAGAGGUUGCAGUGAGCCUA | |
| | GACAGAGCCACUGCACUCCAGCCUGGGUGACACAGCGAGACACUGUCUAA | |
| | AAAAUAAAAUAAAAUAAAAUGUAAUUGAGUUAUAAAAAAUAAAUAAACCU | |
| | GAGACAUACCUUUAAUUGGAUUUCUCAAAUAAUUCUCAAGUAUCUGUACA | |
| | UUGAUAAAUUAAUUGGGAAUUACUCAUUUCUCCUUCUGAGGUGCCAAGGG | |
| | CUAAGGGUUAUACAUGAACCGGAAGGUUUUCAUUUUAUUCAUUAGUUCCU | |
| | UCCUAGGCACCUGACAGGGCAAAUAGAUUCUAUUUGCCUUUUCUCCUGUU | |
| | CUCCUGGCUGUCUCUCUCAAAUAGUAUACUUUGCUGAAAUAUAGCAUAGG | |
| | AUUUAUUUUUACUGUGCUUAUGUUUACUUUUUCUUAGAUAGCAUUGGCUA | |
| | UUUCCUAUCUUGUAGGCUCAUCUUUGAUUUCAAUAUUUGGCAAAGUGUCU | |
| | CAUUUUUUUUUUUUUGGCAGUUUGGCUCUAAAAACAAACUUUCCUCCUCU | |
| | UGCCUUAAACAUUUUUUUUUUCAGAAAUAAGUAAUUCAGACUUUCAAAGA | |
| | GUCCGAUUAUCUUUUAAAUUUUAUAUUUCAAUUGGCUGUCUUUGGUAUUC | |
| | CUAUCUUUUUUCUAGAGUAGGUAGAAUUAACAGACUUUACAUAUUUUUGU | |
| | GAACAUCACAUCUUAGAAUGAUUCUCAAAAUUUCUUUACUUGGGAACUUG | |
| | GCUGUCAUAGUAGGGAAAGUGAAAAAAGUAAUGUGGAAUCCCACGUGAUG | |
| | GCACAUAAUUUGGCAAUAAUAAUUGGAAAGUAAUUAUAAUUGCACUAUUG | |
| | UACUGCUUUAGGUAAGUUUAUGUCAACAUUUCUUUAUCUAAACUCCAGUU | |
| | CACAUAUAGCUAUAUCUGGGUUGUGAAAUUUGCUGUAAUAUUUUUAGAAA | |
| | GAAAUUCCUAAAAUGCUUACUAUAUUUUGGCAGUUUUGAAAAGUAAAUAU | |
| | GAGAAGUAUCUUUUGUAACUUCAAAUUGCUGGGGUUUUUUUUGCCAAUAC | |
| | AUCACUAUGAAUAAUGGCAAAAUCACAUAUUUUCAUGUCCCAGUUUUAAA | |
| | UCAUAUUAUUGCUGGAGGAAAGUCCCAGCUUUGUUUUCAUAAUUCAGCAA | |
| | AUAAUAGGUAGCUUAUAGCCAUAUUUUCUCCUGUGACAUUACUAAUAGUU | |
| | UGUCUUCUCAAAUAGGGCAGUUAAAAUAUCUUUAGAUAUUAAAACAUGCA | |
| | UUAGAGCCUACUUGAACAAAAUUACAAUUUUGUAAUUGAAUUAAAGAUCA | |
| | UUCCAUUGGGAAUCAAAAAACCUGGGUUUUAGUUUUAGCUCUAACAUCAG | |
| | CAGCUAAUAUGAUUUUAGGUAAGUUGCUUAAAUUCUCUAGCUCUGUUACC | |
| | UUGUCUUUGGAAUGAGAAGCUGAACAAGGUGAUUACUAGGAUUCAGUGCA | |
| | AAUCUAAAAUUCUAUAACUGUGCUUUUGUUCAUAAUAUAUUUUAGUUAAU | |
| | UAUUGAAAUGUUCUGCUUUCCAGAGUAGUACCGUAAACCAGUAAUUUAAA | |
| | CCCUUCCUACUAUGUUUCAACCACUUUCCCAAGGGCUGGUAUGGGUCUUA | |
| | CUUGCUCCUACAUGUAGGAGUUAGUCUGCUCUUAUUUAGUCCUUUAGAUA | |
| | UUUAGCACAUUUUAGCCUGAAUAUCUGACACAGAUGAAUGAUUUUUUUUU | |
| | UUUUUUGAGACAAAGGCACACUGUCACCAAGGCUGGAGUGCAGUGGCGUG | |
| | AUCUUGGCUCACUGCAACCUCCACCUGCUUGGUUCAAGUGAUUCUCGUGC | |
| | CUCAUCCUCCUGAGUAGCUGGGAUUACAGGUGUGUGCCGUCAAGCCUGGG | |
| | UAAUUUUUGUAUUUUUUUAAGGAGAGACGAGGUUUCCCCAUGUUGGCCAG | |
| | ACUGGUCUCAAACUCCUGGCCUCAACUGAUCCGCCUGCCUCGGCCUCCCA | |
| | AAGUGUUGGGAUUACAGGCAUGAGCCACCACGCUCAGCCCAAAUGAUUUU | |
| | AUAAGCGUAGAAGACAUCUCUCACAUCUUAGUGGGAGAAUUAAAUGUCUC | |
| | UCUUUUUCACUGAAGUCUUACCGAGAAUCAUUUCUUUUCUGUCACUCUUG | |
| | CUGAUCUUGCUGAUGAGCCCUAGUUCUGCUUUGUCAAACUUAUUUGAGAU | |
| | CAGGCUGGCCCUUGUGACAAUCAUUGCCGGUAUAGACUUUUCAUAACAUC | |
| | CAGAAACUUCAAGCACCACUAUAUUGUUUCUACUUCCUCAUAUGCCUUUU | |
| | AAAGUAUAGAAGCUGUAUUCCAAUAUAUAUCUACAUGUUAGGUUACAGUG | |
| | AUCUGCUUUUGGACUUGAGGCAUUUUUUGGACUCGCCUGUCCGUAAGUCU | |
| | CACUUGCAUUCUUUGUGAACAAAGAAUACACCCACUCUCAAAUGUAUAUG | |
| | CCCACACACAAGCAUACAGUAUCUUGUACACCUAUCUGCACACACACAAA | |
| | CACAUGCAUAGUGCUUGACUUUGGACUCUGUGCUUUAACUGAGAGUAUUC | |
| | AUUUUUCCUGGUUGUUACCUUCAUUCCAGUUUUGUGAGCUUUGCUGUCAA | |
| | AGUGGCUGUGCAUCUAGCAUAGAGUGGCUGCUGUUGGCAACCCAUGAACA | |
| | AUGAGCUUAAACAGAAGAAUGAGGGUAAUGUUUAUUGUAAUAAAGAUUGG | |
| | GCAUUAAAUUCCUUGGGGGAAUGAACCAAAUCCAAUGUUUUCCUGUUCGC | |
| | UGACAUUUUUUAUAUUGAUGCUGUUUUGGAUCUUCUGGAGAAGGGAGGAA | |
| | AAAAAAACUUCAGCAAAUCAGCUCUUUCAUUCAAAAAUGGAAUUCAGAAG | |
| | GCUAUUUUAGCUAGCUUUUUUUGACAAUCCUGGAUUUAAUCAGUUUUUAC | |
| | UGUGAUGAAGACAGUUAGCCUCACAAAUUUUGUGGAAAUCUAAAGCGAUG | |
| | AAGGGUGUUGUUAAGGGGGAAACAGAGUGCAAUUGUUUAAAUAAUCCUGG | |
| | GCCAAUAGCAUUUGGUAGGUGGAGUUACUGUUGUGUGCAUUACAGAGCAA | |
| | GAUAAUUCUGAAUGAAGACUUUAAACAUUACACAUGCUGGACGGAUUCUG | |
| | GGUCUGCUCUUUGUGCAAACAAAACAAGUGACUAGGUUUUGCUUACUAUC | |
| | CAUUAGUGAAUGCAGCUUGUUUAAUAAGAUUUUUUUUAAAAAAAUGCAUG | |
| | UAAAGGAUUUAUCAAAAGCCUUUAUGAAUAUUUCAUGAGUUGAUAUAUUC | |
| | AGCUGAAUGAAUUCAGUGAGUGUCAGUGUGUAGCUUGCAGACAAACCUCA | |
| | UCCACAAGGAGGCUACUGACAUUGGAAGCACUUUGGCGCAUUUUCAGAGG | |
| | CAAAGCCAGCCUGAUAAAGCUCCUUGUGACAGCCUGACUUGCUAUUCUUC | |
| | GAGUAUGCUGCUCUUGCUCUAAGACGCUCAUACAUUGGAGUCACAGCUUC | |
| | GUAAAUUAAACUGGAAAUAGUUUGCAGACUUAUCAGUCUUCUGACCUGCA | |
| | GCAGUGUCUCUGCUAUGUUUGAGAUUUUUGUUUUGGAUGGUGAAAGCUAG | |
| | CACUCCUUACAAGACAUGACAGCAAAAGAUUCUUCAAAGGAACUUACUGC | |
| | UUCUGAACCUGAGGUUUGCAUAAAGACUUUCAAGGAGCAAAUGCAUUUAG | |
| | AACUUGAGCUUCCGAGAUUACCAGGAAACAGACCUACAUCUCCUAAAAUU | |
| | UCUCCACGCAGUUCACCAAGGAACUCACCAUGCUUUUUCAGAAAGUUGCU | |
| | GGUGAAUAAAAGCAUUCGGCAGCGUCGUCGCUUCACUGUGGCUCAUACAU | |
| | GGUAAGAUAAGACUUGGAGGUAUCUACAGUGCUUUUCAGAUUCUCGCAUU | |
| | AGCACCAGUGAUCUAGCAGUGUAGAAAAUGGAACAUUUGAGUUUUGUUCC | |
| | UAAAACAUACUGCAAGUGUGAAAUGAAUGGAGACCAGGUCAGAUGAAGAG | |
| | UACAUAAAUGCACUGUGUGCUAAAUUGUCAUUGUGCUACUAGCUUUUUCC | |
| | AGAUAAAAUAUGCUUUUAACUGUAGAGUCUUUAAAAUUACUGCUUAUCAG | |
| | AAUACAUAGCUAAUUCUAGGUAAGUCGAGUAAUAAAUGAAUUUUUAAAGU | |
| | CAGUUUUUUGUUUCAUAUCUGUUGAGGUGGUAGGUGACUUUUUUGUUUUG | |
| | UGAUUGUUAGUGAUUUUAAAAAAAUAAUUUUGGGACAUGAAUAAGUGUUU | |
| | UAAUCUUAGUUCUAACUUAUUUUUAAGAUAAUGUAUAAAGUCUAACUUAA | |
| | UGUAUUGUACCAUCAUGUUUAUAGCAUAUAAACCAGUGAGUUUUUGAUUU | |
| | AUUGACUUAUCUUUACAAUACUUAAGGAAAGGGAAAACUAAGUUCCCACU | |
| | UCUGAAAUUGCAUGGUCAGUUACUUUAAUUUUUGAUUCCAAAAUGCUGUU | |
| | AUCUUUAAGAGCUUCUUUUUUUAAAAAAAUGGACUUUAAAAUUUAUAGCU | |
| | AUUUUUUAAAUACCCAGCAAAUAAAUUCAAGAACCCUCUGUAAAACUUGC | |
| | ACUGAGCCUGCCACCUUUAAUAUACAUGUUUACAAGGUAUGUAAAUAUAG | |
| | UAGGGUAACGUCCUUAAACAGUAUAAUGAUGAAAUGUAAGGAAAAAACAC | |
| | GAUGAAAUUAAAUCACUUUUACUGUGAAAGCCAGGCCACUUCAGAUUUUU | |
| | UUGGAGGCUAUAUUUUCUUUCAGAUUAAGGUACCUUUAAAAAUUGAGUAG | |
| | CUGUACCUUAGUUUUUUCUAGCAGGGACCAUAGCAAUAUAGGGAGCAUGU | |
| | ACAUUUUAAAGGCAAUUCUAACACAGUGAUUUUGUGACUACAUGCUGGGU | |
| | UGCCAAUCAACAUUAUAAAUUGAGGAGAUCUGAUUUUUAAAAAUUAAUAU | |
| | UCACUUGAAUUAUUUUCUACUUAUCUAAAAUGUUUGUCUGCUUAAUCUAC | |
| | UGUAUAGUGCAUUGGCAAAUUAAAGUUACGCCUAGAAAUAUAUAGCUUGC | |
| | CUUUGAUAGAUACCACAUGCUUAGCCAACACAUACUUUGCCUGGCAUAUA | |
| | UGAUGUGCUUAUUCUUGCUUUCUGACAAUGGACUGUUCAUAUAAUGGCUU | |
| | UAUUGCAGAUUUUCUUUGGUCAUCUUCUUGAUGUAUUACAUCAUGUUGUU | |
| | UAAUUGUAAUCUCUUAUUUGCAGAAUACACGUGGUUAGUCUUUCAAAGCC | |
| | UACUAAUGAUGGUAACAUUGUAUAAAGGGAAGGUUUAUGUAUAAGGAUGU | |
| | ACUGACUUGAAGAAGUCCUCUCUUUUGUGCUUUAUCCCACCCCUAACAUG | |
| | UAUGUAUUUUCUUAUAGUUUGCUGAGUAGUAUAACUGAAUAGGGAAGAUA | |
| | UUAGUUAUGAAAAUAAUAUAUACAGAAAAUACACAAUUAAUAGAUGCACA | |
| | UUUUUUCUUGAGUUAAUAUUAAAUUGCUAUUUUAACCAAAAGGAUAUUUA | |
| | AUUUUAGAUAGCUAAUUUUUAGAUAAUGAUAAGUCUUCUUAAACCCUUGG | |
| | UAAUAUUUUUCUUUAUGUAGAGUUUUGGUUCUGAACACAAUAGUGUGACC | |
| | UUUCUUUUUCUUAUAAUAGAUUUUUGAUGUGCUAAGAACAUAGAGCUAUA | |
| | AAUACUGGAGUUUUUUACUUUUCCAAAGAACCCCAAUUUUAAAAUGAUUA | |
| | AAGAGCUAGUUUUACUCAUGCAUUAUACAAAUUUUCUGGCAGCAAAAGGA | |
| | ACAAUUAAAUGGUAUCUGAAGUUACCUACGCUAAUAAGAUGAUAUUCUUU | |
| | UAGUUGAGUUAAAACAAAAUGUUACAAUUGCCAAAAAAAAUAGGACUUUG | |
| | AUAUAAUGCUCUAUAAUAUAGAUUAAAAUAAUUCAUUAAUCAUUAAACUU | |
| | GUGAAAUAACUUUAGUUUUGACUAUGAUUCAUUUUCACCCUUUUUUCUUU | |
| | CAACUCACACAUUCCCCUUCUGAUGUUUCUUAGUAAAACAGUUUCACAGU | |
| | UUGUUUUCAGCUCAUGACUAAGUCUUUAUUUAUAUUUAUUUCUAUUGUUU | |
| | UAUCUGAAUACAUGCUUAACAUUUUUCAAAUUUAUUUUCAUACAACAUGC | |
| | AAUGAAAAUCUCUUUCUAGAAAAUAGACAUGUAUCUUACAUAUUUUUAUG | |
| | ACUUUUUCAAAUCUUAAAAAUUUUGCCUUUCCCGGGGAACCCCGAUAAAG | |
| | UCUCUAGCCUAAUGCUUCAUGCUUUCCCCUUGCUUCGCCUUUUUGUCUCU | |
| | UGAGCACCCUAACGUCUUUCCAAUGAAUAUAGAUGGCUCUGUAUGGUCUG | |
| | CUCUACCUCCUGUCUCUAAGACUCGUGAGAAUAAUAAACUUUUCUUUCUU | |
| | GAGUUUCCAAAAAAUUUCAUGUGCUUUUUUACAUAGAUGAAAUGGUUAUA | |
| | UGAGGAAAUAAGAGCAUAAGUACUUUAUUUUUGCCUCUUUGUCAUAGUGA | |
| | AAUGUAACCAGCCUUUCACGAUCAACAAUUGGUAUUUUUCCAAACCAUGC | |
| | ACUUAAGUUAGAAAUUCCAAGAUAUGCACUUAGAAGUCUCUAAAAACAUG | |
| | UUAUUACUUUACAGUAGACAGUAGGCUUCUUUUAAGAAGAUUGGAUUGUU | |
| | UUGAUUCCAGCAUUGAAAUUAAAGUAAUUUUCAUGAUGAAGCUAAACAAU | |
| | GCAGAGGAUGCAGUUUAUAUGCAAACCCACUAAACAGUUUUUAUUUGUAG | |
| | AUUUCAUAUUUCAGUCAGAUGGAUGUGUAGUUCUGGCUUUAAAAACCCAA | |
| | AUUAUGAACUGUGGACUAAUGGCAGAAGAUUUAAGAAAUAUUUAUGAAAU | |
| | UCUGACUCUGGGCUAUUUAAAAAUAUCUGGGGGAAAUGUGGAUAUUCAAU | |
| | GUGUUAUCACUAAUCAGAGUAUUUACAAAAUCCUAAAUCUGGAAGGAAUU | |
| | UGGAUGAUUAUCUCAUUGCUUCCUUUUACCCUUGGGCAGGACCACAAUUA | |
| | CUCCAUCUUUAACAUAUGGGAAUCUGCCCUACUUUUAAGACUCUAGAGAA | |
| | AAUAAUUCUAAAAUCUCACUUAGUAUACAAUCUUACUACUUUAUAGUCCA | |
| | GGUACUAGAAAAUUAUUUGUUAUGAUUUAUCUAUAUUAUUUAUAUAUUAU | |
| | AUAUAAUCUUUUUUCCCCUCAGUGGAUAUUAUUAAUAAAAAAGAUGCGUA | |
| | AGAGCCUGCAUGAAUCUGAAGAUCAUUAUUAACUGGUGCCAGCGUUUUCU | |
| | UUUUCAUUCACUCAACAUAUGUAAUUGAGCACUGCUGUGAGCAAGAAGAG | |
| | AAAAAAAAUCUGAUUCAACCCACAUGUAUUCCAACACCUACUAUAUUUUA | |
| | AGUUUUUUACUAGAAUGUCUGCAUAUGUUAUCCCAUUUAAGUUUCAUUUG | |
| | GAAUAUAAGUAUACUCCAAAUUCAUGAUCCUUUCAAUAUUACCUGCUGCA | |
| | GAUAUCAAAAGUAUUUUGAAUUGCUCUAGCUUCUUAUAGGUGUUUAUACU | |
| | CCAUUCAUCAUUAUUUUGACUCUGAAUCACUAUCUCAAAUUUCAAAAUUU | |
| | UUCUUUGAAUUUUGAAACCCAAAAGUGGAUACCAUACUCUUUGUUAGACG | |
| | AUAUGAGAAAAUAUAGAUAUGAAACAUGUAAUAAGUUGCAAAGUGUCAAU | |
| | GGAACCUUCAGAAUUACUGAAUUGGAAAAUUACUGCCAGAGUUCUCAGGU | |
| | CAGCAUAGUAUUUAUCCCCAAUCCCACAUCAGGGAUGACAACUUAUGAAG | |
| | CACAGUGUUAUCCUACUUGGCCUGUAUGGCCUGCCUGUGCUUGCCUGGUA | |
| | CUUGUCACACAGUGUCCAGCUUUUAUAACCUUCCCUUCUUUGUUGAUAUC | |
| | CCAACUUCAGUCGUCAGUUCUCUUUCAUCAUUAGCAACUUGAUGCUCUCU | |
| | GAAGAAAGCUACUGCAAUGGUCUUGAAUAUAUCCUGGGCCCACUGCACCC | |
| | CCCUGACCCACCCCCGCCCCCCACCCUGCUGCAUUGCUGCUCCUUUUUGG | |
| | AGCUCUCUUCUUGUAUUUGUGAGGACUACCCUCUAUCUCUCUGUCCAUGC | |
| | UUGCACACAGGAAACAAUAUUUUUGUAAUUAGAAGUUAGUAGGAGAAAAG | |
| | AGGCUUGUAUUCCUUCUUCUCGUUUAGUUUUACCUGUAUCUUGGAAAUCU | |
| | UUUCUCGUUUUAACAAAGGUUUAUGAAGUCUUUACUUGUCUAUCUAAUAU | |
| | UUGAAUACAAAAGUUUUAAAAGCCAUAGUUAGUACUCUAAAGGGACUUAU | |
| | AUUUGGCAAGACAACACUGGAUUAAGUGGAGAAUAGGAAAAAAAACAGUG | |
| | AAUAUGGCAUAUGAUUGACCUUAGAACUGCCGACAUGCUUGCUGUAUUAU | |
| | ACAACUUAGUCCUAGUUUGUUAGCUAACUCUACUGAAUCUGUGCUAGUAA | |
| | UAUUGAUCAAUUGCCUUGAAUUCUAUCCUGGUAAUAUUGGAAAUUUUUUG | |
| | AGAGAUGUCUGUCUUUACAUGUUUAUUGAUUCAACAUUUGUUUGAACAAA | |
| | ACAGUUGUUAAGAGCCUACUAUGCACCAGGCUCUGAUCUGGGCUUAGGAG | |
| | ACACAAAAUGAGAAAAAGCCCUUGUCUUCAAAGAACUCCUAUACUAGCUG | |
| | GAAGACAAACUUAUGCAAUAAUAAUUAACCAAUGGUGUGGGAAUAAUGCU | |
| | ACUGAAGCAUGUUGUUCAACUUGUCAUGAGGGCACAGAAGAAAUAACUAC | |
| | CACUAGAGAGUGAGUCAAUGUGUUAAGGGAAGUUUCGAGAAGAGAUGACA | |
| | UUUGUGUGGGGUUGUGACAAAUGAAUAGGAGUUUUCUAGGAGAAAAGUGG | |
| | GCAGGAAAGGUAUUUUUUGGCCAAGGGAGCAAUAAUUAGGAAAUCCUGUU | |
| | AACAAAUAAUAACAAUAAUAGCUCUAAUAUAUUCAGCACUUAUAUAAUGU | |
| | GUUGGAUAUUAAUUAUAAAUGUAUUUACUCAUUUAAGCAUAUAAAACCCC | |
| | CUGAGUUAUAUACUAUUAUAUUAUUUUCAUUUUAUAGGAAAAGGAAUAAA | |
| | GGUCAGACAGGACAAGUAAUUUGUUCAAGUUUACACAGAAAAUAAACAGC | |
| | UGAGUUGGGAUUUGACUCUGAGCACGAGUGAAAAUACACAUAGUUCACUG | |
| | GGGUUUCAGCCUAAGGAUGCAAGGUGGGAAAGCUAGGAAGUAAGACUGUG | |
| | CCAGCAGAUUGGAGCCAUUUGCUAGGGCUUCCAAUAUCAUGAACAAGAGU | |
| | UGGAGUUUUAGCCCAUCCAUUCUCGGUGAGGAGCCACGACAGGUUAUGAG | |
| | AAGGGGAUUGCCAUGUAUUAUUUUUAUUUUGGAAAGGUACUUUGCAGGUC | |
| | AGGUAGAGAAUGGAUUAGAGGACAAGGAGCUUAAAGGUAACUGGAAAAGG | |
| | GACAAUGUGCAUUUGAGAGAUAAUGACUUCUACUUAUUUUGUAUAUCCCG | |
| | UAACAUUGAAAACUAUGCAUAUGACAUGUUUUAUGACUUAACAAAUUAUG | |
| | GUUUAUGGAAACUGGGAGGAAAGCAUGUGAACUUGGUGUCUCUUGCAUUU | |
| | AUCACUUACACCAUGAUGCUGUAGAUAGUACUAGCUGGUACAAUACCCCC | |
| | ACAUGCAUCAAACAAUCAGCAAAGGUGCAUAGGAGGAUGUGUGUAUCUGA | |
| | GGAAAACCCUAUUAGCUCAGUCCUAAUUUACUAGAGGUGCUUUGUAUUUU | |
| | ACUUUAAGUGCAGACUCCUAGUGGGUUAAAAAAAAAAAAAAAGCAGAUAU | |
| | UAUCUUAGUCCACAAUCUGAAUCAAAUGGCAGUAGAAAUCUAUGCCUCAU | |
| | AGCCUGAGUCUGUUGCUUUAAUCAGAGCAAGCAUUUUAUCAGUACAUCUA | |
| | UAAAUAGUGUUUUCCUGGCCUUUUAGUGUCAUCAAGUCUUCAAUAUAUUG | |
| | GAUUUUUAAAAUACUUACAGUAAAAAGGGCUUUGCAAAGUAAUGGUGACA | |
| | ACCCCCAGGACACAUGCCGGAAGGUGGCCCAGCAAGCUUGCCAGCUGCCU | |
| | UUCUGCUAAAGGCUAAACCCAUUUUGCUAAAUCCCACUUGCGUUCUCUUU | |
| | CCAUUCCUUUCUGUUUUGUUAUCUCUCAGUUUUUCUCCUUGGAUGUUUAU | |
| | AGUGCUUCCUUCUCUAACAUCACUGUCAAUGCACUUGCAACAUUAUGCUA | |
| | GAAGCAUUUAAGCGAUUUCUUAGAAAAAAAUCUCAAACGCUUACCAUGGU | |
| | UUUCAGGCCCUACAUUAUCUGGCCCCAGGCUGCUUCCAACCUCCUCUCCU | |
| | GUUCUCCAGCCCUGUGAGCCUGCUUUCUGUUGUUCAGCAAACUAAGCCUG | |
| | UAUUCACUUCUGGCCUUUGCACUUGGUUUUCUUCUGCCUGGAAGAAAAGU | |
| | ACCUGGAUGUUAACACGGUUUCUCCUUAUGUCCAUUCAGGAUUCUACUAU | |
| | CUCCAGGUAUCACUAUCCGGAGAGGCCUUUGCUGACCUCCCUAUCUAAAA | |
| | UAGCACCCUUUCGCCUUUUACUUCCUUGUCUUUUUGGUACUUAUUAUUAC | |
| | CUGAAAAUACAUUAUAGACAUGCUUAUUUGUUAUAUUGUCUUAUCCUUUC | |
| | AUCAUCUGCCAUCCCUGCUAAAUUUCAUGAGGGCAGUGAUUCUGUCUUGU | |
| | UUUCAGUAUCCUCAGAGCAUACAAGAAAGGCAGUCAUUUAACAAAUACUU | |
| | GUUGAAGAAAUAAAGAAUGAAUAUGGUUUCUGUUUCCUGCUCUUUAUUCU | |
| | AUGAGCAUUUUUCCUAUAGCUUGAUAUGAUGGAAAUGGAAAGGAAGCUUU | |
| | GUUGAUACUUACUCUAUAAGCAUUCUUCUCCAAUCAUCUCUCAAUAGCUG | |
| | UGCUCUCUUUCUGUCAGUAGAGCUACUGCAGCUUGCUUCCUAUAUUGUAU | |
| | UCUAUGAUAUUGCAGGUAUAGAUUUAAUUAUAAUAUCUCACUAGAGCCUA | |
| | AAUGCUUUACAUAGUAGCCUAUUUAAUUAAGUCACCAUGUACAAAUCAUA | |
| | CCUCCUAGUCUGUAGUGCUGUAAAGUAAAUAUUUGUCUGUAGCACUGUAC | |
| | AAUUAUUUAUUUGGUUUUUCUUCAUUCAUCAAGUAGCAUGGUUGGUGCAU | |
| | CAGCUCAGGAAAGCUCUACAUAUCUCUCACCAAAUGCCUGAGGAGAAGUG | |
| | UUGUAUGGGGGAAUCUACUUGUUAUUUUGCCUUCAAAAUAUUUACAUAGU | |
| | GUACAUUAGAAUGCUUAAUUAUCUAUAUUUUUCCACUGAUGCAACAUUUA | |
| | CAAUAUUAACUCUAGCAGCAGCUACAGCAAUUUGAGCUUUUAAUAGGAUU | |
| | UCCCCAGGGAACUUGGAAGUAAGAAGUCUAUGACUUGCAGAGUGAGGAAA | |
| | UUGCAAGUUAAUAACCCUUGUACUCUAGAAAAGACGUCACUGAGAGGGAA | |
| | ACUGUUCACUUGGUUCUGCCUCAUUUUUUCUUUCCUGAACUAGGAGGUUG | |
| | GGGACCUCUGUCACUGGUAGUAAUUUGUUUGACAUAAAUAUGUUUGAAUA | |
| | CCACUGUGUACUUUAGGUUGAAAUGAAAAAUAACUCAACUUCUCAGGUUG | |
| | CUUCUUCCUGGAACUUAGGGAGAAUGAGGAUGACUGCUUCUCAAAACCUU | |
| | UGUAAAGAGAAUGCCUAUGAGAUGAUGUGCUCCCUUGACAGACCAAGGCU | |
| | UGUCCUCCUCUUCCAAACUAUACCUCUCAAAACUCAGCUUUAUUGCAAAC | |
| | UAUGCAGAUUGGAAGUCAGGGGAUGGAGGAUAGUGUAGCUUCAAAGAGUG | |
| | CUUCUUUCAAACUGUAUUUUCUGGAAAAUCAGUGAUCUAGGAGCCAUUUG | |
| | GAGAUUCCUCGAAAAAGAGGGUGGUGAUGAUCUGUUUCCUGGUUCUCGGU | |
| | CAGAACAAAGCAACAUGGAUGAUUAUAUGCUUGGAGAAUUUUGGAGGAUU | |
| | CUCACACAAAUUCUAUAAUCUCCAAGUACUUGAAAACCACCAGUUUAAGA | |
| | GUUAAAUGAGUAGAGGGAGAAAAGAAAUCUGUUUAGUUGUGCUUGAUAAA | |
| | GUCACAGACAUUUCCAGAUAAUUUAUAAUAUUAAAAUCAUCUUCAUCUAA | |
| | UAUGUCCAGGAAUGGCAGCAGGAUAAAUGAGGAGGAUGAAAGCAACUGUG | |
| | GGUCAGGGAGACAUCACUGGUAGUGAACAUUUAAAAUUUCAAAGUUGAAA | |
| | GAAUAGUACAAUGAACACCUUUUACCUAGAUAUACUAAUUGUUAGUGGUU | |
| | UCUCUUAUUUACCUCUUUGUCUCUUUCUGUGUAUAAAAUCAUAUAUAUGU | |
| | UUUCUUUAUGUUGAAGAGAGCACAUUUCGAUGGAGAGCAAUAGUUUUGGG | |
| | AUCAAAAGAAAAAUAUUAUUUAUUUUAAAACAGUUUCCAUGUAUCAAGCU | |
| | UGCUGUUAUUACAAUUCAUCCGUGUUUUUGCAUUUAUUAGUAAUUUAUUC | |
| | CCUUUUAUUAUUAGGUGGUAUUCCUUUGUAUUGGGUGUACUACAACUUGU | |
| | UUAUCUGUUCACCAACUGGAUCAAAAUUUGGAUUUCCAGUCUUUUGGCUA | |
| | UUAUGAAUAAAGGUGAUAUAAACAUUCAUGUAUGGUUUUCUGGGUAAACA | |
| | UAUAUUCUAAUCUUUCUUGGGUAAAUAUCUUAGAGUGGCAUUGAUGGGUU | |
| | GUAUUGUGAAGUAUAUGCUUUUUUGGUUGUUAUUUGUUUUUAUUUUUUUG | |
| | ACAUGGAAUCUUGCUCUGUCACCCAGGCUGGAGUGCAGUGGCAUGAUCUU | |
| | GGCUUACUGCAACCUCUGCCUCAUGGGUUCAAACUAGUCUUGUGCCUCAG | |
| | CCUCCUGAGUAGCUGGGACUACAGGCACGUGCCACCACACCCAGCUCAUU | |
| | UUUGUAUUUUUUGUAGAGAUAGGUUUUCACCAUGUUGCCCAGGCUGGUCU | |
| | AGACCUCCUGGGCUCAAGCAAUCUGCCUGCCUCAACCUCCCAAAAUGCUG | |
| | UGAUUACAGGCAUGAGCCACCGUGCCUGGCCUAUAGUAUAUGCUUUACUU | |
| | UAUAAAACACUGCCAAACUGUUUUCCAUCAUGGAUGUCCCACUUUAUAGU | |
| | CUUACUAGCUAUAUAUGAGAGUUCUUAUUAUUCUGUAUGUGUGAGGGCAU | |
| | UUUGUAUUGUCAAGUUUUUUUUAAAUUUUCGUCAUUCUAACAGAUAAGUA | |
| | GUGGUAACUUACUGUGAUUUUAAUUUGUGUUUCCCUUAUGACUAGUAAUA | |
| | CCGAACAUCUUUUAAUGUCUUUAUUUGCUAUCCUUAUCUCUUUGGUAAAA | |
| | UAAAAUUGUUUAUAUAAGUCAUGUAUCAAUAAACUUUGCAAAUAUUUUAA | |
| | UUUUUAAGAGCACACUGACAUAUUGGCAAGUUAUGAAUACAUUUGUAGCA | |
| | CCGGUUUAAAAAAGAAAACUAUUAAAAUAUAGUAGAGGGCUAAAGGCAUC | |
| | UAUCAUAACAUGAUUUUUCUUGCUGAUAAAUUAUUGGUUUUCAUUUCUUA | |
| | AAAUGGUUUCUAAGAGAUGAUGUCUAUAAGAUUCAAAUUUUCUUCCUCAA | |
| | GAAGGAAGUUUUUUUAAAAAAAUAAAUCUAUAAUAAUUUUAUAAUAUAUA | |
| | UGAAACACUAUAUAAUGCCUUAAUGUAGUAUACUGUAGAACUUGGGGUUC | |
| | UAUCACUGUAUAAUUUUGACGUUGUCUGGAAUUUAUUUUUCAUGGUGAAU | |
| | CUUUGUUUUAUGUGACUUCAUCUGGAGUUGAGAAUGCUAGGUUGGGUAUC | |
| | AGUAUUUUACCUUGAUUUGCUCUUGAAACUUUAGAUCAAUCCUAGAACUA | |
| | AUUUAAUUGUCCUAGACCUCAAUUCUUUAACUGGCAAAAUGGAGAAAAGA | |
| | GUUUUAUAUUCAGAUAUUUGCAAAUGUUAAUUUAAUUUAAAAAAUAUCUA | |
| | CCUAGACUGUGAGUUACCUAUUUCUUGAGAUAAUGUAUUAAAGUACUUAG | |
| | AACAGUACUUCAAGCAUUAUAAGUUGUUUAAUAGAUGUUACUGUUUAAUU | |
| | AUCUGACUAAAUCUCUAAACAUACACAAACAUUCAUAUUAUUGUCAGGGU | |
| | UGGUUGGUAAAGCAGGAACUAGUUUUUUUUUAUUUUUUGAAAAGCAUCAG | |
| | UAUGUAUGGACUGUGCCAAUUUGGAAGAGUCAAAGUGCAUGUGGGUUAGU | |
| | AAAGAAGUACAUUUAUAAUAUCCACAGUGUGACAAUUCAGUAUACUAAGA | |
| | AACAAAGUAUCAUUCAUUUGCUGUCAUGUAAUAAUCAUAUCCUAAUGAAC | |
| | CAAGCUAAAUUAUACUAAAAUGCACUAAUUAUCAGAAGACCCAGUGUGAA | |
| | UAGCUUACAUUCCCGAUAAGAAAUUUUAUUGUCUUGUUUUGCUCUCAUCA | |
| | AUACAUUUAAAAAAAAAAAACAUUUAUCAUCUUCACUCUGUGAAAGAGAU | |
| | AGGAAGAGAACCACUUUGAAGUCAUUCCAUCCACUUGUACCAAUAAGCUU | |
| | AAAAGUAACAAGACAAAGCAAAACAUAAGAAGAUGCCUUGUCUAUUUCAG | |
| | GAUCCGCAUAUUAAAAGCCAAUAUAGGAUUCAGUGAACAAAUGCCUUGGA | |
| | AGAAUUCCAGUGUCAUCAAACUUAUUUAUCUCAAAACGUCUCCUUCCAUC | |
| | CUUUCUGAUCCUCUACAAAAUUAAUAAGCUGAGACUAUUUGUUGCAGCAC | |
| | AGAGGAUCUGAGCAUGCUUCUGUGGCUUAUUUCUGCUGUAAUUCCCUCAG | |
| | CAGUUUAUACAGGAAUUCUUUAUCUCACCAUCAGGAUUCUUUAGGCCAGA | |
| | AGAGAGGUCAGAGAAGACUGGACAAAUAAACUAUGGGAUCUAAGUCUUUC | |
| | UUGCUAAGGUCCUAACUGCCCAAGACUUCCUCCUGAGAGUCUACUGUUGA | |
| | AAUUCAUAUGCAAAUUUCAGUUUCAUGGGAUUAACCCUGGCUGUUGUCUU | |
| | UACCUGUCAGCCUGCUCAGCCAGGUGUGAGUUCACCCAUAAAACAGUGUC | |
| | UUUCACAUGCUCAUUUAUCAAAACGACAUAGAGACUUUAGGAUGCCAGGU | |
| | GAUAUGAUGCAAAAAGCACAACAUUUGGUUUCUAACUGCAUCUUUUACCA | |
| | UACACUGUUUUUGGGCAAACCAUUCAACCUAUCUUAGAAAAAGUAGAAAA | |
| | AUUAGAAAAAUUAGAAAAACUCUACUUGUAUUUCAUUAGGCCUUUAUGAA | |
| | GAUUAAAUGAAGUAAUGUGUGUAUGAUGGUUAGUAUAAUCCCUGGCCAAU | |
| | GGCAGGUAAUAAAAGCAGUCCUUAGUUCACUAAUGUAUCGAGAGCUUAUG | |
| | AUUUUGUGUAAAAUUUAUAUAAUAUGUAUCAUAAUGUAAACUACUUAAAG | |
| | AUCUUUAUAACAUGGUUUAAUUAAAUCAGUUUCAUUGAGCAAAUGUGGUG | |
| | CAUCUUAAAUCCAUUCUUUUAGACCACUUAGAUAUUACUUAGUUUCAUAG | |
| | GAUAUGACUUUAUCAUAAAGCACAUAUUAAUUAUCCUUACCACAAUAAAA | |
| | UAAAAAGUAAUAAAUCCUUAUGUGUUAUGGUGUGUAAUCACUCUCAUUCA | |
| | UCACAGAGACCAUUCCAAAUUGCAGAUUUUGACCAUAGAAUAAUCUUUUU | |
| | UCUUUAAAAUUCCAGUUACCUUCUUGAAUUUGAUACUUUCUCCAUUAGUG | |
| | AAACUAUUAAUAGAUACCAAUGAAGCACUGUUUUUUUUAAGUUAACGUAA | |
| | AAAACAUUUAAUGGUCUUUUCAAAGUGCCAGAACAUCUCUGAAGUUGUUU | |
| | UCCUUAGUGGGAUCUAACCUCCUAGUGACUUGUUUAUAUCAGAAAGUUUA | |
| | UUCAUGAGAGGCAGUGGAAAAGCACCUAGGGAUCUAGAUUGACAGAAUCU | |
| | AGACAUGUUUCCUGGCACCACCACUUACAAACUGUUUAUUUAUUUGUUUA | |
| | UUUAUUUAUUUAUUUUUUGAGUGGAGAAGGAGUAGUUCUUUAACCUUUGG | |
| | GAGCAUCAACUUCCUCAUCAGUAAAUAAGAGAUAAAACAAUAUCACUGAU | |
| | UUCUCUACUUAUAUGAUUUUAUAUGAUUAUAUAACAUUUACAAAUAUUAA | |
| | UUAGUAUGGUUGAGAAGCAUGGGAAAAGAAAGAUAAAGAUUACUUUACAG | |
| | AAAGUAAGGGUACUCCAUCAUUAAAAUAUAAAAAUCAAAGAGCUUGACUU | |
| | AAGUGCCGCAGGUGUAUUUCGGUAUAUGUAAAGAGCAUCCCAUUAAUAAA | |
| | UAUUAUCAAAUACCAUACACAUUGCUCCAGAUUUAUUUUAUAAAAGGGGG | |
| | AAGAGUUUCUGCUUUAUGGGACCAUGUAAGUGAGAGACUGGAGACAGGAG | |
| | ACAGACUAAGUGGUACCUUAAGAUCUCAUCUACCCCACGGGUCUUUAUAU | |
| | CACUUUUAGCUUCCUGCAGAAAGAUAAUAACAAGCUCAUAUAACCAAUAU | |
| | AUAAUGAAGUGAAAGUUGCCUUUAUUCAGACAACUUUACAGCAUUUAAAU | |
| | CAAAUUGCUCUUUAUUUCUUGGUCAUUUCAAAGAAUAUUUAAGGAUGAAA | |
| | ACAAAAAUAAAGGUUAUUGCUUCUGCUUUGAGAAAGUUUUCCUUCUCACA | |
| | UAAUCUUUGAUCAGGCUAUUGUGUCACUUGGCAACUUAAAGGCAGGAAAA | |
| | AGUUAUAUCUUUAAGUCCAGGGAUUCUAUUUAAAUGAAUUGCCAUUGAAA | |
| | AGUGGUUAUUCAAAUCAACUCACAGCUUAGUAACAUUGAAAAAUAAAGUA | |
| | AACUACUUUUGGGUUUCACAAGGAUCAUGCCAAGUUUCUGGGUCUGUCUA | |
| | AAUUAAUAAAAAUAAGACUUAGGAACCUGUAUGGAAAUUGUAGAUUAAAU | |
| | UAAGCAACUGCUUCAUCUGAAUAAAGCUUUGCAGAGUCUAAUAUUAUGAA | |
| | GGAGCUAUCUUCAUGAAUACAGUGCUUGCUUGGGAUUCAAGGGAUUUGAG | |
| | UUUUAAUGCCAGCUGAAUCCCUAAUGCCCACGGAGCACAUAUGCAUUCAU | |
| | UUGAUGCUGGUAGCUUUUCUGUCAAGUGGGAUUAAUAAUUACCUGCUCUA | |
| | CCACCCUUACCUUAUGUAUUAUAGUAUGAAUAUUCAAUACCAGUGUUCAC | |
| | GGAAUGCUCUGCCUUUCUGCAAAAUAACAUGGCAGAAGCAUUUAGAAAGG | |
| | AAAAAAAGUUUCUAAAAAAGUUUUAUGCGUAUCAGAUUUUCAAAAAUGUG | |
| | UCUCAGCUUUAAAAUACAUUAAUGAAUUUUGUAUUUUAUAUGAACCCUAA | |
| | AAAUGCUCUUGCCAAGUACUUAGUUAUCUGAAAGCUAAGAUAAUUGUUUU | |
| | GGGUUUCUUACAGCUUUGCUGCAUUUGUGUAUGUUAGUCCUUUGUCAAUG | |
| | GAUAAACACCAGAUUGACAUCGCUUAGUGAGUUAACUCUGAUGGGUGGCU | |
| | AGUAUUUUCGUUUUUUAUUGAUUACAACCAACAGAUAUUUAUUGCUGGAU | |
| | AAUGCAAACAUGAGGCACCAUGCCAUCAAGGAGCUUUAAUCUGGGUAAGG | |
| | AGAGUAUAUAGUCAUUUGGCAUGUCAAAUGAUUAAUUUUUCACAGAUACA | |
| | AGUGUUAAGAGUAUAUCAGUAUAGCCAGGCAUGGUGGUUUGCACCUGUAA | |
| | UUCCAACCACUUGGGAGGCUGAGGCGAGAAAAUUGCUUGAGGCCAGAAGU | |
| | UUGAGACAAGCCUGGGCAACAUGGUGAGUGAUAUGGUUUGGCUGUAUCUC | |
| | CACCCAAAUCUCAUCUUGAAUUAUAGUUCCCUUAAUCUUCAUGUGUCAUG | |
| | GGAGGGACCUGGUGGGAGGUAAUUGAAUCAUGGAGGAGGUUCCCCCAUGC | |
| | UGUUCUCAUGAUAGUGGGUGAGUUCUCAACAGAUCUGAUGACUUUAUGAG | |
| | GGGCUUUUCCCUGCUCUGCUCUGCACUUUUCCUUCCUGCAAUCAUGUGAA | |
| | GAAGGACGUGUUUGCUUCCUCUUCUGCCAUGAUUGUAAGUUUCCCGAGGC | |
| | CUCCUCAGCUAUGCGAAAUUGUGAGUCAAUUAAACCUCUUUCCUUUAUAA | |
| | AUUACCCAGUCUCAGGUAUUUCCUAACAGCAGUGUGAGAAUAGACUAAUA | |
| | CAGUGAGACUCCCAUCUCUACAAAAAUUAAAAAAAUAAUAAAUUAGCCAG | |
| | GCAUAGUGGCAAGUGCUUGUAGUGCAAGCUACUCUUGAGGCUGAGGUGGG | |
| | AAGAUCCUUGAGCCCAGCAAUUCCAGGCUUUAGUGAGCCCUGAUUGCAUC | |
| | ACUGCACUCUAGCUUGGGCAACAGAGUGAAACUUUGACUCUGAAAAAAAU | |
| | AUAUAAAAGUAUAAAAUACAGGGCCAGGCAUGGUGGCUCAUGCCUGUAAU | |
| | CCCAGCACUUUGGGAGGUUGAGGCAGGGGGAUCAUUUGAGGUCAGGCAUU | |
| | CAAGACUAGCCUGACCAACAUGGUGAAACCCCAUCUCUAAUAAAAUGCAA | |
| | AAAUUAGCCAGGUGUGGUGGCAGGCACCUGUAAUUUCAGCUACUUGGGAG | |
| | GCUGAGGUGGGAGAAUUGCUUGAACUCUGGAGGUGGAGUUUGCAGUGAGC | |
| | CGAGAUCAUGCCACUGCACUUCAGCCUGGGUGACAGAACAAGAAAAACGA | |
| | ACAAAACAACCCCCCCCAACCAAAACCAAACAAAUGAAAAACAAGUAUAA | |
| | AAUACAAUGCUCUCCCACUGCUACCAGGUGGAAAUGAUCUAAUUAUGAAA | |
| | CUACAGGUUUGAAGAGCAAUGGUUCCACUUCUUUUUCCAAAUGGCUGUGA | |
| | GCCUUGAGUCUGCCCCUGUUAUGGUGACAUACUGAGCAAUUAAUCAGUAG | |
| | UGAGCUAUACUUGAGAAUCUGGUGUACUAGCAAUAGGCUAGGUAGUCUGC | |
| | UGAUGUGAGCAGGUAGGAAAUAUCUCCUAAGACAGAGGGAAACAAAUUCG | |
| | CAUUUUAAUUUUUUUCACAUAAAAAUUAGGCAAUGAUAGAACAAUUACAU | |
| | CAUAUAUUUCUUAUAUCAUUUAGUCUUCAUCUCCAUUUGCAGAUGAGAAA | |
| | GUUGAGACUCAGGAAGGUCGAUUAAUUGAUACCAGGUAAACAAGCUACAA | |
| | AGAAAUAAACCAAGAUUUAGUACAUGUGUUCUGAUUUGGAGUAACACAUU | |
| | UUAUAUGCUGACAAGGUCCAUAGAUACAGAGGUAUAUCAUUACACAACCC | |
| | GCCCCAUCCCCUUUGCAACUUCCAACUCCCAGCUCCUGUGGGAAAAUAUC | |
| | CAGGGUUACUGGAUUCACAUGUUCCCUUAUUAAAAGCUUCCUCCCAGGAA | |
| | GGCUUGUAAAAUUCAUUUGCUCUUACACAUAAUGGACUCAUGGGGUUUAA | |
| | UUUGGGGAGAAUAAACCAUUAAACUAGAAGAAUUAAUUUCUACUUACAGA | |
| | AUUUGGCACAUUAGAGAGAAGAGACAAAGGGGAGUAUUUUGGAGGAUGAA | |
| | AGGUUAUGUUCCAGGCAUGGAACAUAGACGAUGGGCUGAAUGUCCAGGCA | |
| | UGUCAUUGUAUUUGCAAACCAUUCAUUCACUCAUUCAUUCAUUCAUUAGC | |
| | AUUGUCAACCACCUGCUUGAUUGUGCCCAUUUCCUGCCUUUCCUGUUUCU | |
| | AUAGAUGAAUUAUCUGUGUACCUGGUUAAGGCCACCCCUUACUGUGCACU | |
| | GCAUUAUAUCAGUACUCACCCACAAGGGCUUUGCUCUUGCACUUGUCCCC | |
| | GCUGUCUCCUGUAUCAUCAGUUUUCCAUUCUAUUAAAUGGUAUCCAUUUA | |
| | UAAACAUGCUAUUUCCCUUUAAAAAGCCCUCUUUGACCCGAGGCACUCCU | |
| | CUAUCUAUUGCUGUAUUUCAUUUCUCCCUUAUAAGCAAAACUCUCCCAAA | |
| | UAAUUGUACUGCUCUACUGGUUUCCUCUCCUCCAGUCAUCUUCUGGAAAC | |
| | UUCUAACCAAGUUUUCUCCUCACAAUUCCACUAAAAGUGUUCUUCCUGAG | |
| | AUUAUGACAUUCCAUGUGCCAAAUAUAAUGACCAAUUCCUUCACUUGGCC | |
| | UCCAGAACACUGAAUGUCUUUCCUUCCUCAUGGGCUUCUCUUUCUCGGUU | |
| | GGUUCCUCCUCAUGUUCCUCAAGUCUAAAUAUUGGAAUGCUCCAGAGGGU | |
| | UGUGGACCUCUUCAUUUUUGUCCUAUAAUAACUCCCUAGGCCAAGUAUCU | |
| | CAAUCUAAAUAACAUCCGUAUGCUGAUGCUUCCCAAAUUUAUAUCUCUAG | |
| | CCAGACUUUUCCUCUGAACUCUAGAUCUAUGUCUCUCUCCAGCAGCCUUC | |
| | UUGACAGCCAAAACUUAGCACAUCCAAAAUGGACAUCAUGGCUUUCUCCC | |
| | CCCUACACCAUGCCCUUGAUGUAACAUGUUCCAAAUCUUUUCCAUUGCAG | |
| | UAAAAUGCAAUUCAAUUUUUCUAAUUACUCAGGUCAGAAAUCUUGGAGUC | |
| | UUGCAUGAUUCCUGUUUUUCUCUGAUAUUUCCCAUUUCAAAUGCAUCAGC | |
| | AAAUCCUAUCAGCUCAACAUUCACAAGGAAGCGAGACUGUCACCACUUCU | |
| | CAUCACCAGCUGUCGCUGUUCUGAUCCAAGUAACCAUUACCUCAAACCUG | |
| | GAUUAUCAAAUUAACAUCUUACUGGAUCUCCCUUCUUUCAUUCUUUCCCC | |
| | UUUACAAUUAAUUCCUCUUGUAGCUGCCACAGUGAUCCUUUAUAUUAUCU | |
| | GAUCAGUUGUUUCAUUCCUCUAUUCAGAAUCCUUUAACAACAUCCUAUUU | |
| | CACUCAGAGUAUGGGUCAAUGUGCAUGUACCAUGCCUACAAAUCUACCCU | |
| | GGUCUCUACCUAUUACUUCUCCAACUCCUUUCCUUCCCCUCACACCAUCC | |
| | CAGCCACACUGCUGCACUUGGUUCUCAGGCUAAGCAGGAUUUCUUAUCAA | |
| | GACCUCGGCCUUUUCUUGUUGUCAAGAAAAAACUUUCUCCAGAUAUCUGU | |
| | AUGGGCUUUUCUUUUCUUAUUUCAGGUGGUGUCUUGAAUUCAUCUAAUUG | |
| | GAGAAGACUUUUUGGCCAUCUAUAAAAAAUAGCAGUGCACAUCUUUGCCU | |
| | UUCUACCAUCAUUAUCCCUGAUUUGGGUUCUUUAUAGCACCUGAUGUAUA | |
| | UAUCUACAUAUAUAUGUAUUUUUCUAUAUCUACAUCUAUUUCAUAUCCAU | |
| | AUGAUCUGUAUCUUUAUCUAUCUAUCUAUCUAUCUAUCUAUCUAUCUAUC | |
| | UAUCUAUCUAUCAUCUAUCUAUCUAAUCUUUGGUAUUGUCUGUCUCUUUU | |
| | CUCUCUCCAAUUAGAAUAUAAUCUCCAUGAAGACAAAGAACUUUGUCUAU | |
| | UUUAUUCAUUUCUAAAUCCUCAAAACUAGAACACAUGGUAGCACAUGGUA | |
| | GGUAGUUAAUACAUAUUUAUUGAAUGAAUAAGUGAAUAAAUAGACAAAUG | |
| | AGUUGCAGACUGUUUGGGUACCUUCCAGUUUUAAGCACUGAGGCCCCAUG | |
| | UUGAAUGGGAGAGGGAAAUGUGAACAGAUAAUUUAAAAUAUUAUGUGAUA | |
| | AAUAAAAUAGUAAAAAUAACUAUUGCCUUUCAAAGUAAUUUUAUCUGGAA | |
| | AUAUAAAUUUUAAAAUUUAAAAAAUAUUAUAUAAAAUUUAAUAAGAGAUU | |
| | AUUUUCUUUAUAAAGAGAAAGAGGGGCAUUUGGCAAGUAUACUAAACUUA | |
| | AUAGCAAGAAAACUUAAAUUCAUAUUGUGAUUACAUUGUUUACUAGCUGU | |
| | AGAAUCUUAGGAAAAUAACUCAUCUAGUCUCUUCCUCCCAUUGUACCUAG | |
| | UAUACUAUCUAAUUUAUUUUAUAGUUGUGAAGAUUUAGUCAGAUAAUAUC | |
| | AUACAUAAUGGGCAUUAAGUAGAUAUUAUUUUUAUUUCUUUCCAUAUCUA | |
| | AUACUAUACUUUACAUUCAUAGGUUAACCUUCUGUGAUCUGUCUUAUUCC | |
| | UCUGCAAUUUUUCAAUGACUUUCUAAAUUCCCACUUCAUUGACUUUCUUC | |
| | CAUUUUCUUCCUGUGAGACUUUUUGACAUCAUGUGAUGCCAUACUCAGCC | |
| | UCAUCAUCCCCACGAAGAUUAAUCAUCUUUCUCUCCUUGCUUCAACAAUA | |
| | UUGCAUGUGAUAUUUAGCUCUAUUUCUAUGGAUGGAAGCAAGGAGAGAAA | |
| | GAUGAUUAAUCUUCGUGGGGAUGAUGAGGCUGAGUAUGGCAUCACAUGAU | |
| | GUCAAAAAGUCUCACAGGAAGAAAAUGGAAGAAAGUCAAGGAAGUGGGAA | |
| | UUUUGAAAGUCAUUGAAAAAUUGUGGAGGAAUAAGACUGAUCACAGAAGU | |
| | AACCUAUGAAAGUAAAGGAAUCAUAUAAUGUGUACUCUUUUGUCUAUUUU | |
| | UUUCACUCAUCAGAAUUACUUUGAGAUUCAUGCAUGUUGUUUUAUGUAUG | |
| | CAUAGUUAAUCCUUUUUAUUGCUGAACACUAUUCCAUUGCAUGGAUAUAC | |
| | CAAAAUGUGUUUAUUCAUUCACUUGUUGACAAGCAUUUGUUUUUAUGUUU | |
| | UUUUGCUAUUACAAAUAAAACAUUCAUCUGCCAAAAAAAAAAAAAAAAGA | |
| | ACUGGUGAGUUCUUUAGCUCUAUUUCUAUGGAUGGAAAGCUGGAUUAAUU | |
| | UUCAACAUUGUAGGACAGUAAUAAGUGAAUAUUUAUUCUUUAAUUGGAAU | |
| | UAUGUUUCUUUGAGUUGCUUGUCACUAAUACGUAGUGAAUCAAUGGAAAU | |
| | GAAAAGGACACUCAUAAAGUAACUGCAUAGUGAGUACAUAAACCAAAACC | |
| | AUGGCUAUCUUUUUACCCUGUGCUACAGCAAGGCUAGUGAAACAAGAGCA | |
| | ACAUGAAUAUUUUUAAGAAAGAGUAUCACAAUGGUAAAUUUUAUUUGUAG | |
| | GGCAUUAUGUAGAAGAGAAAAUUUUCAGGAAUAAUUAUAUUAGACUCAUC | |
| | UCUUUGAUGACAGACACUAUUUCCUAUUCAUAUUGCAAAAUAAUUGGCAA | |
| | GUACAUGUUUUAAAAAAAUGAGUGUUUUUUUUCCUGUGAGACAUCUGUAU | |
| | UUUACUACAGGUGGGACAAAGUUAUUUCGGUCCUUAUUUUGCCACUAGGA | |
| | UAUUGGCAGGCAGAGGGUGACAAAAUAAUGUAACAUCUGAAAUUUCAGGC | |
| | UAUGAGGAAACUCACAUACAUACAAUUUGUCAGACAAUUAGGAUAUUUCU | |
| | AAAAUGUGAACAGAUGUAUAAUAGAAAUUAGCAAAAUGAAAAAUGACCUU | |
| | UUUUUGUUUUUGAUGGAUACAUAAAGAAUUUAUAUGCAGUUUUCUAGGUU | |
| | AGCCUAGUUAAAACCUCUUCUUUCUACAUAUUCAGUUAAGAAAUAAUUGU | |
| | UAAUUGAGACCAAGACAUUAUAAUUGUUUCCCUUUUGUAUUAUAAUAAUG | |
| | AAAAAUAUCAAGAAUUAUAUCUUAUAACUUUUUACACAGGUCCAAGUGAU | |
| | GUUUUCUGAACUUGCAUGCCACAUUUUUUUAGCCAAGUGUUCACCAGUUG | |
| | GGAACUCCUAGGGGUUGUUUAUUUUUCAGUGAAAGACAAUCUUUUAAGGA | |
| | GUUUAUGUCCUAAAUAUUUUCUGGUUUUGCAGUAGCAUACAUCAGACAUA | |
| | GUCAUUGUGUUUGGAUUGGGAUUUUCACUUUUAACAUGUUCUCAAAUUCU | |
| | CUAGGCGUUUAAUGUUUCCAUCUGGUCCUCAUAAAAGGUUUCACAUUUGU | |
| | AAUGCAGCUCAGGCUCCUACUCAGAGAGCAUCAUUAUAAUUCUAUCCAUG | |
| | ACUCUAGUUUGAUAGAGCAGAUACUUUUAAAACUUUGGAUUUGUUUUUUG | |
| | CUUUUGUGUUCUGUCUUAAACCUAGGAAUAACACUUGAGUUUAAUGGUUG | |
| | UUUCUCUCUGAAAAUAAUCAUAGUGUGCACACCAUGACAACAGGCUCACA | |
| | CCCCACUUCCUUCUGUGCCGUUUGAAGCUCCUAGGGCAGAAGGAGUAACC | |
| | ACAUUGUGAGCCCAGUAUGAGGAAACUUAGCAGUCGUCUUACUUUCUCUU | |
| | CAUUAAACAAUCUGUUCUCAGUCAUCACAGAUGGAGAGGAUAUUUUCAAG | |
| | GACUUGAACAUUUUCUUGAAAUAGCCAAACAACUAGGAAUCUCCUUUUGC | |
| | CAUGUUAAAUAAUAGGUUCUAGGGCACAAUUUGAGCAAAGUUCACAGCUU | |
| | CUAGCAAGGUCUUUUUAUGUAGUUAGGUGUGCAGUGCAUGUGGAUUUGUU | |
| | UAGUGAGUAUGUACUUAAGUAACACUAUAUGUCAGGCAAUGCUCUAACUA | |
| | AACUUCACAAAUAGUAACUCAUUUAAUCCUCACAGUAACUUUAUGAAUAC | |
| | GGUACUAUUAGUAUCCCCAUCCAAGGAUGUAGAAACUGAGGCAUAAGGCA | |
| | AAAAGACCACAGCUAGCAAGUGGAAGAGCCAGAAUUAAAACCUAGGUGGU | |
| | CUGGCUCAAGAAUCCAUGGUCUUAACUGUGAAACUGUGCUAUGACUGAAU | |
| | GUGUUCAGUGUCAGAUUUUCAAAGAUCUAUUAUUAUCUUCCCAGAAUUGA | |
| | AUUGCACACUUUGUGUAACUCUCCCCAACACCAUUCUUGAUUCAUAAUUG | |
| | GUACUCAGUGAAUGUUUUUUUACCUUAGGAUUUAAUUAAGAGUGAGUGAA | |
| | AUGAUAACUGAUUUUGCCAUCAUUUGAAAAAAAGGCAGAACCAAAGGUGA | |
| | AUCAUAAUGGUAGCCAGUGGACAGGCAGUUUUUCAUUCAGAAGUGUCAAU | |
| | GGAUCCCGUUAUCCAAGGAUAACCUAUAUUUAAGGCCCAGAUACUUCUAA | |
| | GAUGAUGCUUUGGAUUCUACAUUUUCAAAUCUGGAAGAACUAUCUUCAUU | |
| | UCACUAAUUGUUCUUUACUGAUACAGUUUUUUUAAAUUUAAGUGUGGUGA | |
| | AACACAACCCAAAAUUUACCAUCUUCGCUAUUUCUAGGUGUACAGUUCAG | |
| | UAGUGUUACGUAAUUCACAUUCUUGUGCAACCAGCCUCCAGAACUCUUUU | |
| | CAUCUUUCAAAACUAAAAGUCUAUACCACUUAAGCAACCACUCCUCGGUU | |
| | UUUCUUCCCUCUAAGCUCUCAGAAACCACCACUUUUUAAAAAAAAAAUAA | |
| | CUCAUCCACCUAUGGAAAUUUAGGUUGCUUCCACCUUUUGGCUAUUGUGA | |
| | AUAAUGCUGUUAUGAACAUGGAUGUAAAAAUAUCUCUUUGAGACUCUACU | |
| | UUUAUUUCUUUGGGGUAUAUACCCAGAAAUGGAAGUGCUGAGUCAUAUGA | |
| | UAAUUCUAUUUUUAAUUUAUUUCCCAUACGGUUCUCCAUAAUGACUGUAU | |
| | CAUUUGACAUUCCCACCAACAGUGCAUAAAAGUUCACUUUCUCCACAUUC | |
| | UGACCAAUAUUUAUUAUUUUCUGUUAUUUUUUUCUGAUAGAAGUUAUCCU | |
| | AAUGGGUGUUAGAUGGCACAUCAUCUUGGCUUUGAUUUGUAUUUUCCUAA | |
| | UGAUUCGUGAUGUUGAGCAGCUUUUCACGUGAUUAUUGGCCAUUUAGUAU | |
| | CUUCUCUGGAAAAUGUCUGUUUAAGUCCUUUGUACAUUUUAAAAUUGGCU | |
| | UGCUGUGUUGUUGUUGAGUUGUAGGAAUUCUUUAUAUAUUCUAGGUAUUA | |
| | GCCCAUUGUCAGAUAUAUGAUUUGCAAAUAUUGUCUCCCUUCUGUGGACU | |
| | GGCUUUUCACUAUACUCUUGUGUUCUCUGAUGCACAGAAUUUUUACAUUU | |
| | UUAUGUAGCCUAAUUUAUCUACUUUCACUUUUUUGCCUAUGCUUUCUGGU | |
| | GUCAUAGCCAAAAAAUCAUUAUCAAAUCUAGUGUCAUGAACCUUUUCCCC | |
| | UAUGUUUCCUUAUAGAAGUUUUAUAGUUUGGGUCUUGUGUUUAGGUUUUU | |
| | GAUCCAUUUUGAGUUGAUUUUUGCAUAUCGUGUAAGAUUAGAGUCCAACU | |
| | GCUUUCUUUUGCAGUAUCCAGUAUCUUUUGCAGUAUGUUUUGUUGGUACC | |
| | CAGCUCUCCCAACACCAUUUGUUAAAGAAAAAAACCAUACAGAUUUAAGG | |
| | GAAACUCAGACCACAUAGCUAUAAACCUGGAGUUCCUCUCAUACUUUGGU | |
| | CCAAACCAUAAUAGAUUUAGAAAGAAUCACGACUAAAGUUCUCAACUGGG | |
| | GGCUCUUUUCUCCUGGCGAGAACAUUUAGCAGUAGGUGGGGACAUUUUUG | |
| | GUUGUCACAUCUUGGGGUGGGAGUGGGGGUAGCAGUUGUUACUGGAAUCU | |
| | AGUGAGUAGAGGUUACGAAUGUUGCUAAAUACCCUGCAAUAAACAGCUCC | |
| | CUAAAGCAAAGAAGUAUCUGGCUUCAAAUGUCAGUAGUGCUGAGGUUGAG | |
| | GAACCCUGGCCUAGAGUAUAGGGAAGUACAGGUUAUGUUGUAGGACUGGA | |
| | AAAUUGGGGAAUCUCCUUUUUUUGCCAUUUGAGGGCUUGAUGAUCAUUGC | |
| | ACAGGUGACUACCUAAGUGUCCAAUUAUUGACUACUUGGUAGUUGGUUUU | |
| | UUAAAGAUACCUGAAUUUCAUAUACAUCCAGUUAGGUUUUAACAACAGAA | |
| | UGCUAUUUUGAGCUUAACUUUUGCCUUUAAGACUGUUCUUCUCACUUUCU | |
| | UUCACCAUAAUUUUGUCCAUUUAUGCAUUCAUUUUCUCUUUUCUGUAAGA | |
| | AAACAAGUAAAUAAUUGUAGGAUUGAUCAUAAGAAUUGGAGUCCUAUUAG | |
| | ACUAUACAAUAAUCAAGGAUUACAGAAAUGCCUAGAGAUUGUUUUGGCCU | |
| | AGCUGCUCCUCCUACUUCUGAGCUACUUACAGGAGAAAGAUGCCCAAUUG | |
| | ACAAUAUAAACAUGAAAUUAAUUACUUUAUAAUAAAUAGUUAAUUUAUAA | |
| | CACCCAGGCUUGUGUUUAAAAUUCCAGAGCUUGGCUCUCUGCUGAACUGA | |
| | GCUGGAGUACACGACUUCAGCCCAAAGUAAUUUUAUAUUGGGUGUUUAUG | |
| | GUCAGGGAAACUUCGUUGCAGAGGUCAUGCUUGAGCUGAGACCUGACUGG | |
| | CUAAAAGCCUCAAGCCAUGAGAACAUUUGGAAGAAGAGUAUUUCCACAAG | |
| | AAAAAAAAAACAGAAAAUUAAAGUGCAAAGAUCUUAAGGUAGGAAUAAAC | |
| | UUGGCAUGUUCAAGAAAUAGAAAUAAGGCCAGUGAGGCUGAUGCUCUGAG | |
| | UUACUGAGGAUAACAGAAGUAUGAUGCAACUGCUGAAAUCAAAUUAAUAU | |
| | UUCUUUUUCCAGAGGAAAAGGUGGGGGGUGUUUGAUUAGAACUUCUCCUG | |
| | GUAUAAGGGGAGAUUCUCUCAACCUUGGACACCUGAAGUUGGCAGGUGUG | |
| | UAAAGCAUUGGUUUUGUAUUGCAUGGUUGGUUCACGUUAUCAAUUACUGU | |
| | UUAAGUAUUUAUUUAUUCACUUUUCAAAAACUGCUAAUAAUUUUCUAUAU | |
| | UCUAGGCACCAUUAUAGGGCUAGGCAUUGGAGAAAUUGAGAUGCAUAAGA | |
| | CAAAAUGCUUAUGUUUAGGAGCUCGUAGUGUGCAUUUAUGGAUUGGAUAG | |
| | UUGGAUAGAACUCUUCUAAUUAUUGACUAUGUGACCUUGGGAAAGUUAUU | |
| | AAACCUCUCUGUGUCUGAGUAUCUCCAUCUCUAAAAUGAGAUAUUUAUUG | |
| | CAGUUACUUCAUGGAGUUGUUGUGAGGAUUAAGUGAGUUAAUACCUUUAA | |
| | AGCAGCACCUGAGACGUAAUAACCACUCAGUAACUUAGCUAUUAUUGUUG | |
| | UGGUGGUUUUACAGAAGCACAGGAAGAGACUCAUUAAUUCAGUCUCAAAG | |
| | GUUAGAGAGAAUUUAAAAGAAAAGGUGAAGUUUGAGUUCAUUCAUUAACU | |
| | CAUCAUUUGUUUGAUAAAUAUAUAUCAAGUGCCUACUGUAUUCCACAUAC | |
| | UACUUUUCUGCUACGGCACAUACUUCUUUCUUGCUGCCUGGAUUAUCUUU | |
| | GACACCAUUAUUCAGCUGUCAAACUUUAACAUCCACAUCAUCCUCUUCAA | |
| | UGUAACCUUCUUCAGUUUAAGGAAGAGUAUAUAUUAGAGAUAAAAAUUGU | |
| | ACAAAUAAUAAUUUAGUUACAAUUGUCAACCAUGAUAUCAAGUGUAAUCC | |
| | AGUCAGUGAGAGGAAAAAUGCAAGGAACAUCUAAAAAGUGUGAAAGAUAA | |
| | GGAAAUGUCUUAGCUAAGAGAUGCAAUAUAUGAGAGAUAAGAAAUGCAAA | |
| | AUAUCAUAAGAAAAAAUAAUCAUAAUCAUCAACUUUGUAGGUUUCCCAAA | |
| | CUUCUUAUAGUUUAUGUAUUUAUCUUCUAAUUUUCUAAGAGUUGAGCACU | |
| | UAUAACCAAAAGGGCUUCAAUGCUUGCUUUUGAGUCAGCCAUAAGUGGAU | |
| | AAGGAGCAAAAGGAUGCAGCUUGCUUUCAACGCCAAGUCAGACUUGUGUU | |
| | GGAGGAGGUGUUUAGGGAUGGGUUUUCUAAGGGAGAAGAAUAUGUAGGGC | |
| | ACCUACUGAAGGAGAAGAGUAUGGGCUCUUUGGCCAUUCUUGCAGAGAAA | |
| | ACAACAGAGUGUGGGUGGGAUAAGGAGCAGAACUAGGCUAGAAUAGGGAG | |
| | CAUUAGGUUGGAACAGGAGGGCUGGAGGGUAGUUCUCUGGAAUUUUGAUU | |
| | GUGCCUAUAUAAGCAAUUCUUUGCCUUUUUUUGGAUUUACAUACUUGAUG | |
| | UCUUCUUUCAGACUGUUGAAUAUUAGAGCAAACAUUUGAGAGCAGAGAAU | |
| | UUAUUGUAUUUGUGUAACCAGUAUCUCUCAUUCUGCUGCAUAUAUAUAAG | |
| | GCCUUUAGUAAAUGUUUGCUGAAUUUCCCUAUCAAUUUAUAUAUCAAUUA | |
| | AUGAGAUAAUAAAAUGAGAUUGGAAGAAGAGGAUCAUAUCCAGUAGACCU | |
| | AGCCUAUAAUUUGUUCAAUAUUGAUGUGUUUAAAAAUUAAAAAAGCUACC | |
| | UAGAUAAAAGGCCAAAAUAUAACAUGUAGUUAUGACAGUCAGGGUUUAUU | |
| | UAGCAUUUCUAUAAGCCAGUAGCCUUUCAAUUUAUUCUGCAUGUAUUCAG | |
| | UGGUGGUGGUAGACAACUGGUUUUGAGUCAAAAUUGCAUAUAGGAUCACA | |
| | UUUUCUUGCUAAUUCAACUUUCUUAGUUCUAGGUUGGUAAAUAUUGUAGA | |
| | AAAAGCAAAAUGUUUCCCACAUGUCUACAGAUUUUCUUGCUUUAAUAGCU | |
| | UCAUUUGUGAUUUUGGGAACCAGAAUAUUUUUAAAUAAAACAAGUCAUUU | |
| | AUUCCUGGAAUAAGUCCCUAUGCCAUGCAUGUCUUGAGAUUAGAAACUCA | |
| | GCUGAGAGCAACUAGUGAUUAUUCCCUGACUACAACUAUUUGUGAGAUAC | |
| | AGAAAUCUUAAUGAUGAUAAUCCAGUAUUAGUGAGAUUUUUAAAAAAUAU | |
| | AUAUAUAUAUAUUCAAGGGUGCUUUCCAAGAACAAUUUAUACCUCACCAA | |
| | UUAUUUACUGAGUUGUAUGUGGAAAUUGGAAUUAACUGGCACCAUUUAUU | |
| | UCAGUUACAUUUUGAAACCACUUACAGAUUGACACCUGGGUAACUGGCUA | |
| | UAUUUUAUAUAGCUCUGCACGGAUCAUAGAGUUUUAGAGGUAGAAGUUAG | |
| | UUUUUUCUUUUAGUGUUACUCUUGUAACAGGAUGUAUUUAACUGUUUCAG | |
| | CAAAAGGAAGACAGACGUUUCCAUCUAAAGAGUAUGUGGUUUUAGGCCGG | |
| | GCACGGUGGCUCACACCUGUAAUCCCAGCACUUAGGGAGGCCAAUGCGGG | |
| | CGGAUCACGAGGUCAGGAGGUCGAGACCAUCUUGGCUAACACCGUGAAAC | |
| | CCCGUCUCUACCAAAAAUACAAAAAAUUAGCCGGGCAUGGUGGUGGCGGG | |
| | CGCCUGUAGUCCCAGCUAGUCGGGAGGCUGAGGUAGGAGAACGGCGUGAA | |
| | CCCAGGAGGCGGAGCUUGCAGUGAGCCGAGAUCGUGCCACUGCACUCCAG | |
| | CCUGGGCGACAGGGUGAGACUCUGUCUCACAAAAAAUAAACAAAAUAAAA | |
| | UAAAAAAAGAGUAUGUGGUUUUGACUUUUAUAAUACAGUAGAUUCUGCUU | |
| | GAAUAAAUUUUAAGAUUAAGUAUUUUAAUGUGCUUUGUGUAGAUACAACA | |
| | GAAAUAUCAAAGGACAAUUCCAAUAUGAGGUUAAUAUUUUAAUAUCUUAU | |
| | AUCAAAAUAUCUGAGCACCUAGAAACUGGCAGUGACAAUUAACAGCUUAA | |
| | AUGAUUCUUCAAAUAAUUCAAUUUAAGGGUAGGAUUUGGAAACAAGUAGC | |
| | AUUAUCAGACUGAAAAUAACACCAUUUUAUAAGAAUGUCUAUGGAUACAA | |
| | UCUUAAGUUAGUGGCGUAUGCCUUUUAUAUAUUUAUCCAACCAUUAUCCU | |
| | CUAUGUGCAAAGAAUGUGAUCUAAAGAAUGUGUUAAGGCAGAUAAUACGU | |
| | GUGUAUUAUAAUAAUUAAAAGGUUCUGUGAUACAGACUCUGACCUGAGGA | |
| | UAAAGUACUACAAGAUAUAAUAACUACCAUAAUUUUAUAUUUUAUAAUGA | |
| | UAAAACUACCUAUAGAAUGAAAUACCUUAUAUCUUACUUUUUGCUGCUUU | |
| | UGGGUUAAUUAUGCACUUAAAUAAAUGGAUAGAGUUCUUACUUACCGUUU | |
| | UCACAUUUAAAUUCAUCCUGAUAUGAUGACAUAUAGUUUUAAUGACCUGG | |
| | UUAAUACUGAUGAAAACUUUCAGUGGCAGUGCCUGGGGCCAGUAGUCUGC | |
| | UGCAUAUUUUAUUUUUAUAUUAUUGCAAUAAUAACAAAAGAUAUUGGUGC | |
| | UUCUUUUCACCUACAAAUAAUCAAUUAAGGAACUGAAGCUUAAUUUAUUU | |
| | AAAAAGCAAUUUUUAGAACAAGUCGAUAGUUCACAUAGAGCUAUUUAAAU | |
| | GUCCCAUGCAAAUUGAUGGAAUGUUCUAGCAAAUAUUAUUUAGAAAAUUA | |
| | UUAUUAUUGUUAUUCUUUUUUUUUUUUUUUUUGAGAUGGAGUCUUGCUCU | |
| | GUCACCCAGGCUGGAGUGCAGUGAUGUGAUCUUGGUUUGCUGCAGCCUCU | |
| | GCCUCCCAGGUUCAAGCAAUUCAUCUGCCUCUGCCUCCUGAGUAGCUGGG | |
| | ACUACAGGUGUGCAUUGAAAUGCCUGGCUCAUUUUUGUAUUUUUAGCAAA | |
| | GACAGGGUUUCACCAUGUUGGGCAGGAUGGUCUUGAUCUCCUGACCUUGU | |
| | GAUCCGCCCCCUUGACCUCCCAAAGUGCUGGGAUUACAGGCAUGAGCCAC | |
| | CGUGCCCAGACUAGAAAAUUAUUUUUAAUAGCUCACAUUCUUUGCCCAGU | |
| | CAUUUCACACCGGUAUUCCAUAAGAGUUCCCUUGCAGCCAGAAACAGAAA | |
| | GGAUUAGAUUUUAAUAUCAUCUAACAAUAUUCUUUUGUUGUUUAAUCACA | |
| | CUUUAUAGUGCUAACCCAAAUCUGAUCUGGAAAUCUGUUCAGAUCCAGUU | |
| | UGAGGAACUAGUUGAGUAGACAAUACUCUCAGAGAGCUACAGGAACUAAA | |
| | UGUACAUCUCUUGUUCAUUUGUAGACAUCAUUCAAGCCUCAGGCUUGGAU | |
| | UCUUCGUUUAGGUCUGCCAGUCAACAAGAAUUCACUGGGUGGUUACUCAU | |
| | GACAGAGACCUGUAUGAGACUGUGUACAUAGAAGUCAAAGAAAAAUAGUU | |
| | ACUAUCGACAAGCAUAUAUUGAUACAUAUAGGCACAAUUUUUCUUUCAAA | |
| | AGUUUGCAUUACUGUUGAGGAAAGUAGACAUCUAUAUUUUUAUAAUGCCU | |
| | AUUUGAAAUUUGUCCAGGAAAUUCUGUUGCUGGUCAACAGUGUAGCUAGG | |
| | CCUGGUAUAAGAACAGGUCAGGUACAGUAGGAUUCUGAUUAAGGCAGGUG | |
| | UCUGGCAUAAAAAUAGUGAACGUUAGGAAGGUAGGAUUCCUGGUAUAUAG | |
| | CAUUACUUAAAGAACACUUUGAACUCUUUUUUUUUUAAUAUACUUUAAGC | |
| | UCUACGGUACAUGUGCACAACGUGCAGGUUUGUUACAUAUGUAUACAUGU | |
| | GCCAUGUUGGUGUGCUGCACCCGAUAACUCGUCAUUUAUAUUAGGUAUAU | |
| | CUCCUAAUGCUAUCCCUCCUCCCUCCCCCCACCCCAUGGACAGGCCCUGG | |
| | UGUGUGAUGUUCCCCAACCUGUGUCCAAGUGUUCUCAUUGUUCAAUUCCC | |
| | ACCUAUGAGUCAGAACAUGUGGUGUUUGGUUUUCUGUCCUUGUGAUAGUU | |
| | UGCUGAGAAUGAUGGUUUCCAGCUUCAUCCAUGUCCCUACAAAGGAGAUG | |
| | AACUCAUCCUUUUUUAUGGCUGCAUAGUAUUCCAUGGUGUAUAUGUGCCA | |
| | CAUUUUCUUAAUCCAGUCUAUCAUUGAUGGACAUUUGGGUUGGUUCCAAG | |
| | UCUUUGCUAUUGUGAAUAGUGCCACAAUAAACAUAAGUGUGCAUGUGUCU | |
| | UUAUAGCAGCAUGAUUUAUAAUCCUUUGGGUAUAUGCCCAGUAACGGGAU | |
| | GGCUGGGUCAAAUGGUAUUUCUAGUUCUAGAUCCUUGAGGAAUCGCCAUA | |
| | CUGUCUUCCACAAUGGUUGAACUAGUUUACAGUCUCACCAACAGUGUAAA | |
| | AGCGUUCCUACUCCUCCACAUCCUCUCCAGCACCUGUUGUUUCCUGACUU | |
| | UUUAAUGAUUGCCAUUCUAACUGGUGUGAGAUGGUAUCUCAUUGUGGUUU | |
| | UGAUUUGCAUUUCUCUGAUGGCCAGUGAUGUUGAGCAUUUUUUCAUGUGU | |
| | CUGUUGGCUGCAUAAAUGUCUUCUUUUGAGAAUUGUCUGUUUAUAUCCUU | |
| | UGCCCACUUUUUGAUGGGGUUGUUUGAUUUUUUCUUGUAAAUUUGUUUAA | |
| | GUUCAUUGUAGAUUCUGGAUAUUAGCCCUUUGUCAGAUGAGUAGAUUGCA | |
| | AAAACUUUCUCCCAUUCAAUAAGUUGCCUGUUCACUCUGAUGGUAGUUUC | |
| | UUUGGCUGUGCAGAUGCUCUUUAGUUUAAUUAGAUCCCGUUUGUCAAUUU | |
| | UGGCUUUUGUUGCCAUUGCUUUUGGUGUUUUAGUCAUGAAGUCCUUGCCC | |
| | AUGCCUAUGUCCUGAAUGGUAUUGCCUAGGUUUUCUUCUAGGAUUUUUAU | |
| | GGCUUUAGGUCUAACAUUCAAGUCUUUAAUCCAUCUUGAAUUAAUUUUUG | |
| | UAUAAGAUAUAAGGAAGGGAUCCAGUUUCAGCUUUCUAUAUAUCGCUAGG | |
| | CAGUUUUCCCAGCACCAUUUAUUAAAUAGGGAAUCCUUUCCCCAUUUCUU | |
| | GUUUUUGUCAGGUUUGUCAAAGAUCAGAUGGUUGUAGAGGUAUUAUUUCU | |
| | GAGGGCUCUGUUCUGUUCCAUUGAUCUAUAUCUCUGUUUUGGUACCAGUA | |
| | CCAUGCUAUUUUGGUUAUUGUAGCCUUGUAGUAUAGUUUGAAGUCAGGUA | |
| | GCGUGAUGCCUCCAGCUUUGUUCUUUUGGCUUAGGAUUGUCUUGGCAAUG | |
| | CGGGCUCUUUUUUGGUUCCAUAUGAACUUUAAAGUAGUUUUUUCCAAUUC | |
| | UGUGAAGAAAGUCAUUGGUAGCUUGAUGGGGAUGGCAUUGAAUCUAUAAA | |
| | UUACCUUGGGCAAUAUGGCUAUUUUCACAGUAUUGAUACUUCCUAUCCAU | |
| | GAGCAUGGAAUGUUUUUCCAUUUGUUUCUGUCCUCUUUUAUUUUGUUGAG | |
| | CAGUGGUUAGUAGUUCUCCUUGAAGAGGUCCUUCACAUCCCUUGUAAGUU | |
| | GUAUUCCUAGAUAUUUUAUUGUCUUUGAAGCAAUUGUGAGUGGGAGUUCA | |
| | CUCGUGAUUUGGCUCUCUGUCUGUUAUUGGUGUAUAGGAAUGCUUGUGAU | |
| | UUUUGCACAUUGAUUUUGUAUCAUGAGACUUUGCUGAAGUUGCUUAUCAG | |
| | CUUAAGGAGAUUUUGGGCUGAGACGAUGGGGUUUUCUAAAUAUACAAUCA | |
| | UGUCAUCUGCAAACAGGGACAAUUUGACUUCCUCUUUUCCUAAUUGAAUA | |
| | CCCUGUAUUUCUUUCUCUUGCCUGAUUGCCCUGGCCAGAACUUCCAACAC | |
| | UGUUGAAUAGGAGUGGUGAGAGAGGGCAUCCCUGUCUUGUGCCAGUUUUU | |
| | AAAGGGAAUGCUUCCAGUUUGUGCCCAUUCAGCAUGAUAUUGGCUGUGGG | |
| | UUUGUCAUAAAUGGCUUUUAUUAUUUUGAGAUAUAUGCCAUCAGUACCUA | |
| | AUUUAUUUGGAGUUUUUAGCUUGUAGGGCUGUUGAAUUUUGUUGAAGGCC | |
| | CUUUCUGCAUCUAUUGUGAUAAUCAUGUGGUUUUUGUCUUUGAUUCUAUU | |
| | UAUAUGCUGGAUUACGUUUAUUGAUUUGCAUAUGCUGAACUAGCCUUGCA | |
| | UCCCAGGGAUGAAACCAACUUGAUUUUGGUGGAUAAGCUUUUUGAUGUGC | |
| | UGCUGGAUUCGGUUUGCCAGUAUUUUAUUGAGGAUUUUUGCAUCGAUGUU | |
| | CAUCAGGGAUAUUGAUCUAAAAUUCUCUUUUUUUUGUUGUGUCUCUGCCA | |
| | GGCUUUGGUAUCAGGAUGAUGCUGGCCUCAUAAAAUGAAUUAGGGAGGAU | |
| | UCCCUCUUUUUCUAUUGAUUGGAAUAAUUUCAGAAGGAAUGGUACCAGUU | |
| | CCUCUUUGUACCUCUGGUGGAUUUCGGCUGUGAAUCUGUCUGGUCCUGGA | |
| | CUUUUUUUCUUGGUAGGCUAUUAAUUAUUGCCUCAAUUUCAGAACCUGUU | |
| | AUUGGUCUAUUCAGGGAUUCAACUUCUUCCUGGUUUAGUCUUGGACUCUU | |
| | ACCAUUGAUUGUGUGGGGAUUGUAAAUGGUGACAAUCUGUUGGUACCUUG | |
| | GAGAGCUCCCAAUAGCCUCUAUCUUUGAUUUGUUAAGGUGCUACUGAGGC | |
| | UUUUUGUUGUCUUAGCUUUAGGCAUACUUUAUAAGCUCUGUCAUCCUGGU | |
| | GGAUCUAAAAGGUAGUUAAUGCAGCUGUACUUCAAUUUGCUGUGUUAGGC | |
| | UGUGUGGUACUCGGACAUUAUCUACAUUCUGUUAAAACCUUUUGGCCUUC | |
| | ACACCAGAUCUUAGGCAAUAUUUUUGGGUUCCUCAAAGUUUCCUAUGCAC | |
| | UAAGGAGGAUAGAUAGGCUUAUCAGUUCACAAUGUUGGCACACUUUGUCU | |
| | CCAGGGGAAAGAGCAUUUUUUGGCACAGACAAAAGAAGUAUUGCCAAGAA | |
| | CAAGGUAGCAUAUGCUCAGAUCCAAUUAGUGGCAGAGACAAUAAACACAG | |
| | UAAGAGUUCAGAGAAGGAAGUUGUUUCUGAGGGCUAUGGAACAAGUGAAA | |
| | AGCUCCAUGCAAUAGCAGGCCCUAUAGUAAUGAAAGCAUUCAGGUGGAAG | |
| | AGGGAAUUCCAGGUGAAGGAAAUGGUUAACAACAUUCUAUUGAUUUCAAU | |
| | UGAAGAAAAUUACAAGGCAAAAUUUUUAUUUUUAAUUAGCCUAUAAUCCA | |
| | CCUGAGGGAGAAAAGAACCAUGAAAGCAGAAACCAACAUUAGAUCAACUA | |
| | CCAUUUAUGUAGUAAAAAGAGUAAUAAGAUAUAGUGUAAACUUAAUAAUA | |
| | AUGUUUAGGAGAUAAAGCCUUAGGAAUAAAGGAUCUGUGUUGGGUAGGGU | |
| | UAUUUAGGGCACUCUUGAUUUCAGUUAUUAUUUUGUUUCUUAUAUUAGUA | |
| | CUUCAUUACUAUAUGUGUCUCAAGUGGAGAGUUGAACUCAGGAAAACAGC | |
| | CUCAGAAAGAAAAAGAAAAAAAAUACUACAUACUGAUCUCAGUUAUACAU | |
| | AUUUUGAUAAUUAAUAAUGUGAAUUGUUUCAGGCGGUUUAUUUUUUACCU | |
| | UUUGUGCUGCCUACCCACUACUUUUCAUAUUCCAGCCAGCCUCUUGACUU | |
| | UGGUGUUCUCCAUUCUGAGAGACUGUCCAUACAAGUAUCCUCCAUUAAUG | |
| | UGAAGUAGAGUUGCUUAUACUUACAAAAAGUCCUAGAGGAUAGAAAAACA | |
| | AUCAAUAAUGGGUGCAAAUAAUAGAUAAACACUAUUUUUGAUAGUAUUCA | |
| | GUAGGUCUCUUACCAAGUUAUUUUCAGAAUAUCUCUAAAAAAUAUUAAUC | |
| | UAGCAUCACUAUACCUUAGAGUUGGAAGUGCUUCAGAGAUCAUCUCAUUU | |
| | ACCCCUUACUCAAUCAGAUAGCCAUCCAACUUGCAUCACUUCUGUGAUUA | |
| | CUAGGUUCUGUCUGAACAUUUUCAGAUAUGGAAAGCUUGCUACCCCCAAG | |
| | GAAGCCAUUCUGCUGUUGACCAACACUUAGAAAGGUAUUGCAUUAAACUA | |
| | GAAUUUGUCCCUAUGACUUUAAAUAGAUUUUUCCAGUUUCUUUCCAAAGA | |
| | AGCUACACCAACUGUGUCAAUUCAUACGUUUAUUCAACAAAAUGUACAGG | |
| | UCAUAUAAUAUAUGCAAGGAAUUUUCUGAGCCCUACAAAUGAUAACUUGA | |
| | UAAACAUGGAUAUAAUACUGUCUUCUAUGAGAAGGCAGAUUCUUAAUACA | |
| | UUUUUUUGGAUCAGUAAAUAGAUGGAUAGAUGAAUCUUUUUCACCAUUAA | |
| | AUAAUGGUAAACUCUUGAUAUAUAGGUAAAGUUGGUGUAUUCCUUGGUAA | |
| | GUGAAGGAUGUGGAAUGAGCAUUUUAAUAUCCUAAUAUCCUCUAGAAUGG | |
| | UGAUGUAUUUUAUUUCAUAGCCUAUCAGUGUAAAAAAGAGGCAAAUACAA | |
| | UAUUUAUAUAUUUAUUUUCAAAUUAUAAAUAUGAAUUACUUAACUAAAGU | |
| | AUUAUGUAUAUGAACUAAAUAGAUGUUUUUAAAGAUUUAACAUAAGUAUA | |
| | UAUAGCUAUAUAUUUUAAUAGUUUAUACAGUGCCCUAUAUAACCCACAAG | |
| | UAUGAUUUAUAGCUUUGUUAAUUAUUUAAAUAUACUGCCUUUCUAGAAAA | |
| | CCAGCAUUUUAGUUGUCCAUUACAUCUUAGUUGUUUUCCUAACUGGAGUC | |
| | AAGUGAUCACAAUAUGGUAGCAUUUAAGAUUUGGCUUAAGAACAUAUUAG | |
| | AACUCACCAAGUUUUUCAAAAAAUAGGCUUUGAGACAAUAUUAAGAAACA | |
| | GAGUACUUCAAGGAAAGAAGUGAUAGAAGUAAUUUAUGAAAAACAAUUUA | |
| | UAUGAAAGCUUUGUACUUUCAAUAUAAAAGUACUAUAGAAGUAAAACACU | |
| | AUAAAGAUGGAUAAAAAGGAGAAGUUUGAAACUGUCAAGUAAGCUAAAUU | |
| | CCUUAUCUCUUUACAAGCCUAAUAACCCAGUUGGAAUAACUCAUAGAGUA | |
| | UAAAUCUGGGAUCUCAUUAAAAAUAAAUAUUUUCCAGUAACACAAUGAUG | |
| | CAGAGGCAAAAUGUAUGAAAUAGAAAAUAAAAGAAAGAAAAUAAUGACUU | |
| | CAUACUCUUCUUACCUAUUGUCUGCUAUGCCAGUUUGGAUUACCACACCA | |
| | CAGGUUUAGCUUGAGACUGAACUCAUAAUUUCUUCCCAGACUAAUGUUUC | |
| | CAUGUGAGUUUCCUAAUUCUGUGUAUGGUGUCACCAUUCUCUGGGUGUCC | |
| | CAGGCCCCCAACUUUAAACUUAACCCCUGUUCCCCAUUUCAUGACUAAUU | |
| | GUUUACCAAGUUAUUUUGAUUCUCUGCAAUAUCCAGCAAGCAAAUUAUCC | |
| | CAUGUACUUAGGUACAGUAAAAAUUUAGGACUGCUAACAACCACAAAGGU | |
| | UUGUGCUUUAAGUUUAGUUGUGAGCUCUGAAAAAUGUUAGAGUAUUAAGU | |
| | GUCAAACUUAAGAUGGCAAGUCCAUUUUCAUAAUUUAGUUUUUAGUUUCA | |
| | AUUUUGAAGAAUCUCAUCAAAUGUAUAUGUCUCAGAAUACAAACCUUUAA | |
| | UAAUAUAAUUAUUUUUCUGGUCAGUUACAUGUUUUAUUGUUUUCUUGAUC | |
| | CUUAGACUUACAACUUCAAAUAAAAAUGUAAAUAUCUGCAACAUUUGGGC | |
| | AAUAUUGCUUUAUAAAUGAAACUGUACAUACAAGUUCAGUUUUUCACUUU | |
| | UACAAACCUAGGAAAUGCCCUUGAAGCAUGAAUGACAGUGUUUCCUCAAG | |
| | AGGUUCACAUCCUAUUACAGUAGAAGGCAUGGUCAGGACUCAUGCCUACA | |
| | CUUUCAGCUUUGACCCACCCUGGCUUUGGAUCUUGGGUAAAUGGUUAGUC | |
| | UCACUCAGAAUAAAACUUAUACUAGACUUUUCAUGUCAAUGGUCCCUUUC | |
| | UAUGUCUAGCUAACAUUGUGCCUCAUGUCCUCUAUUAUCCCCAUCUGGCC | |
| | CUAAUUAGGUGAGGCUGUAUCAUGGCAGCAUAGCUUCGGGAUUAAGCUCU | |
| | CAGGCUUGAAGUCAGAUUCUUUGGGUAUAUGUCCCUGUUCUGCUACCUAU | |
| | GGUAACAGGACAGAAGCAUUUAAUCUCUAUGUUUUGUUGUCUGAUUUGUA | |
| | AAAUGAGGUAAUAAUAACACUUAAUUUAUGGGUUUGUUGUGAGAAUACCA | |
| | CAGGUUAAUAUAUAAAAGUUCUGAGGACUAGCACAUAAUAACUACUAUGU | |
| | GGUUGCUAUUAUUGAUAUUCAUAUAAAGCACUCAGCAAUGUGCCUGGUAU | |
| | AUAGUGUGUGCUCACUAAAUAUUAACUGUUAUUCUCAUUGCCAGACUCCA | |
| | CUUCAAUUUCUGUAAGCUUUAUUUCAGACUUUGGUCACAAAACAAAAUAG | |
| | AUGAAAGUAUAUCCUCAUAACACAAAUGCGUGUUCUUCACCUCAACAAAC | |
| | CAACCAAUUAUGGCGUCCUGAGGGAGGGUGUGUGCGUCACCUUGCAAUGU | |
| | UGUAACAUAUGCCUGCAAAGGACUAUUCUACAAUACAUGGCUCAGUAAAC | |
| | UUACGUUUUUCUUAACAGCUGCAAACUAAGAUUUGUUUUGAAUAAUCCUA | |
| | UUAGUUUGUGCCAGUGUUACUGUGAAAGUGCAUAUUCAAAUUCUUGGCCU | |
| | UUGUUUUACCUGUUAGCUUUAAUGUAUUUUACUUUUUAAUAUUAUAAUCU | |
| | ACUAAAAUCUAAAAAUAUGUGUAUUAAUCUGUUUCAUGCUGCUGAUAAAG | |
| | ACAUAUCUGAGACUGGGAAGGAGAAGAGGUUUAACUGGACUUACCACAUG | |
| | GCUGCAGAGGCCUCAGAAUCAUGGCUGGAGGAAAAAGGCACUUCUUACAU | |
| | GGCAGCAACAAGAGAAAAAUGAGGAAGAAGCAAAAGCAGAAACCCCCGAU | |
| | AAACCCAUCAGAUUUUGUGAAACUUAUUCCCUAUCACAAGAGUAGCACGG | |
| | GAAAGACUGGCCCCCAUGAGUCAAUUACCUCCCCCUGGGUCCCUCCCACA | |
| | ACAUGUGGAAAUUCUGGGACAUACAAUUCAAAUUGAGAUUUGGAUUCAGA | |
| | CACAGCCAAACCAUAUCAUUCUGUGCCUGGCCCCACCAAAUCUCAUGUCC | |
| | UCACAUUUCAAAACCAAACAUGCCUUCCCAACAAUCCCCUGAAGUCUUAA | |
| | CUCGUGACAGCAUUAACCCAAAAAUCCACAGUCCAAAGUCUCACCUGAGA | |
| | CAAGGCAAGUCCCUUCCACCUAUGAGCCUGUAAAAUCAAAAGCAAGCUAG | |
| | UUACUUCCUAGAUACAAUGGAGGUACAGGCAUUGGGUAAAUAUAGCCAUU | |
| | CCAAAUGGGAGAGAUUGGCCAAAACAAAGGGGUUAUGGGGCCCAUGCAAG | |
| | UCCAAAAUCCAACGGGGCAGUCAAAUUUUAAAGCUCCAAAACGAUCCCUG | |
| | ACUUUAUGUCUCACAUCCAGGUCACACUGAUGCAAGGGGUGGGUUCCCAU | |
| | GGUCUUGGGCAGCUUCACCCCUGUGACUUUGCAGGGUACAGCCCUCCCUC | |
| | CUGGCUGCUUUCACAGGUGGGUGUUGAGUAUCUGCGACUUUUCCAGGCAC | |
| | AUGGUGCAAGCUAUCGGUGGAUCUACCAUUCUAGUGUCUGGAGGAUGGUG | |
| | GCCCUCUUCUCACAGCUCCACUAGACAGUGCCCCAGUAGGGACUCUGUGU | |
| | GGGGGCUCCAACCCCACAUUUCCCUUCUGCAUUGCCCUAGCAGAGGUUCU | |
| | CCAUGAGGGUGCCGCCUCUGCAGCAAACUUUUGCCUGGGCAUGCGUGCAU | |
| | UUUCAUACAUCUUCUGAAAUCUAGGCGGAGGUUCACAAACCUCAGUUCUU | |
| | GAAUUCUGUGCAUCUCUGGGCUCAACAUCAAGUGGAAGUUGCCAAAGCUU | |
| | GGGGAUUCCACCCUCUGAAGCCACAGCCUGAGCUGUUUGUUGGCUCCUUU | |
| | CAGCCAAGGCUGGAGUGGCUGGGACACAGGGCACCAAGUACCUAGGCUAC | |
| | ACACAGCAUAGGGAACCUGGGCCUAGUCCACAAAACCACCUUUUCUUCCU | |
| | AAGCCUCAAGGCCUGUGAUGGGAGGGGCUGCUGUGAAGGUCUCUGAUGUG | |
| | GCCUGGAGACAUUUUCUCCAUGGUUUUGGGGAUUAACAUUAGACUCCUUG | |
| | CUACUUAUGCAAAUUUCUGCAGCCAGCUUGAAUUUCUCCCCAGAAAAUGG | |
| | GUUUUUCUUUUCUAUUGCAUAGUCAGGCUGCAAAUUUUCCAAACUUUUAU | |
| | GCUCUGCUUCCCUUAUAAAAUUGAAUGCCUUUAACAACACCAAAGUCACC | |
| | UCUUGAACAGUUUGCUGCCUUAGAAAUUUCUUCCAUCAGAUACCCUAAAU | |
| | CAUCCUUUUUAGGUUCAAAGUUCCACAAAUCUCUAGGGGAGGGGCAAAAU | |
| | GCCACCAGUCUUUUUGUUAAAACAUAACAAGAGUCACCUUUGCUCCAGUU | |
| | CCCAACAAGUUCCUCAUCUCCAUCUGAGACCACCUCAGCCUGAAUUUUAU | |
| | UGUCCAUAUUGCUAUCAGCAUUUUGGGCAAAGCCAUUCAAGAAGUCUCUA | |
| | GGAAGUUCCAAAUUUUCCCACAUUUUCCUGUCUUCUUCUGAGCCUUUCAA | |
| | ACUGUUCCAACCUCUGCCUGUUACCCAGUUCCAAAGUCACUUCCACAUUU | |
| | UCAGAUAUCUUUUCAGCAACGCCCCACUCUACUGGUACCAAUUUACUGUA | |
| | UUAGUCCUUUUUCACACUGCUUAUAAAGACAUAACCAAGACUGGGAAGGA | |
| | AAAAAAGUUUAUUUAGACUUACAGUUCCACAUGGCUGCAGAGGCCUCAGA | |
| | AUCAUGGUGGGAGGUGAAAGGCACUUCUUACAUAGUGGUGGCAAGAGAAA | |
| | AAUAAGGAAGAAGCAAAAGCAGAAACCCUUAAUAAACCCAUCAGAUUUCG | |
| | UGAGACAUAUUCACUAUCACGAGGGUAGCAUGGGUAAGACCGGCUCCUGU | |
| | GAUUCAAUUACCUCUCCCUGAGUCCCUCCCACAACACAUGGAAAUUCUAG | |
| | GAGAUACAAUUCAAGCUGAGAUUUGGGUGGGGACACAGCCAAACCAUAUC | |
| | UGUAUGUGAUUAGAUAGGAAGCUUGCUCUAAGUUGGUAGAAUGUAAUUUA | |
| | AUGCAAGAGAGUUUGAGUUGAAACAGUUUGAGAUAAAUAUGAUUUAUAUU | |
| | ACAAAUAUUUUACAGCAAACUUUUUCAAUUUGUCAUUAUUGAAAAUGUUU | |
| | CAGAUAAAAAUGAAAGAUGAAGAGUCUAUUGACUCUUUUUUCUUAAGCUA | |
| | AAAUAGACAAUACCAAUAUUAAUUUAUAUAUAGUAUGUAUGAAUGUUACU | |
| | UUUGCUUUUUGAUAUGAAAUAUAAGUCAUAUGUUACUAUAAUAAUAAGCA | |
| | CUACUGACAAAGUUUCUUCUUAAUUGUAAUCUUUACAGCAUAAUGAAUUC | |
| | AUCUUCCUACAUCUAGACAUGCAAUAAUGAAAUAUUUUAAAUACUGAAUU | |
| | UAGGAUGAAUUUGAUUUUAUAUAUGCUUAAAUUUGAAAUAACCACUUUCA | |
| | AAUUUCUCACCCAGUGAUUAUAACAAUAAAUGAUCUUUGCUAGAUAUACA | |
| | AAGGUACUUUCAAGCAAUAUUCCUUUUGUUGCUUACCACAAUUAUGAAGU | |
| | CUAAUUUCAGAAAUCUCAGAUUAAGUGGGAUAGUCAAGCAUAGUGACAGA | |
| | AUUGGUAUUUAAACCUGGACUGCUUCAAUGCCUAAUCUGUUGAGGGUUUU | |
| | UUUUUUUUUUUAUCAUGAAGGGAUGUUGGAUUUUAUUGAAGGCUUUUUCU | |
| | GCAUCUAUUGGGAUGACCAUAUGGUUUUUGGUUUUGAUUCUGUUUAUGGG | |
| | GUGAAUCACAUUUAUUUAUUUGCAUAUGUUGAACCAACCUUGCAUUCCAG | |
| | GAAUAAAGCCUACUUGAUUGUGGUGAAUAAACCUUUUGAUGUGCUGCUGG | |
| | ACCUGGUUUUCUAGUAUUCUGUUGAGGAUUUUUGCAUCUAUGUUCAUCAG | |
| | GGAUAUUGACCUGAAGUUCCCUUUUUUCAGUGUGUCUUUGCCAGAUUUUG | |
| | GUAUCAGGAAGAUGCUGGAUUUGUAGGAUGAGUUAGGGAGAAGCCCCUCC | |
| | UCCUUGACUUUUUGAAAGGGCUUUGGUAGGAUUAAUAUCAGUUCUCCUUG | |
| | CAUGUCUGGUAGAAUUUGGCUGUGAAGCCAUCUGUUCUGGGGCUUCUUUU | |
| | GGUUGGUAGUUUUUGUAGUUUUUUAAAUUACUGAUUCAAUUUGUGAACUU | |
| | UUUAUUGUUCUGUUCAGGUUUUUGCUUUCUUCCUUAUUCAAUCUACUAUU | |
| | UGACUCAGCAAUCCACUACUGGGUAUAUACCCAAAGAAAAAUAAUUAUAU | |
| | CAAAAGACAUAUACACCCAUAUGUUCAUCGCAGUGCUAUUCACAAUAGCA | |
| | AAGACAUGGAAUCAACCUAGGAGCCCAUCAGCAGAGGAUAGGAUAAAGAA | |
| | AAGUGGUACAUAUACAACAUGGAAUACGAUGCAGCCUUAAACAAGAAUGA | |
| | AAUCAUGUCCUCUGCAGCCAACAUGGAUUCAGCUGGAGACCAUUAUCAUA | |
| | AGUGACCUAAUGCAAGAACAGAAAACCAAAAACUGCAUGUUCUCACUUAU | |
| | AAGUGGGGGCUAAGCAUUUAAUACUCAUGAAAGUAAAUACAGGAAUGAUA | |
| | GACCCUGGGGACCACUAGAAGAGGGAGGGAGGGAAGAGGGCAUGAGCUGA | |
| | AGAACCAUCUGUUGGAUAUUAUGCUUUCUGCCUGGGUGACAGGGUCAUUG | |
| | GGACCACAAGCCUCAACCACAGGCAAUUUACCCAUGUAGCAAACCUGCAU | |
| | GUAUAUCCUUUAAUCCAUAAUAAAAGUUGAAAUUAAUAAAAAUAAAAUAA | |
| | AUUGUACAAAAAAACCCUUGGACUUCUAAUGCCAAAGACUACAUUUCCAG | |
| | GUAUUUAAAGUAAAUAGACUAAAAUAAUUGAUUAGAGAAUUUUAGGUGAG | |
| | AGGAAAACACCCUUUCUCUGUGGAUUAAUAAAAACCCAAUUAAUAUUCUU | |
| | UCUUUUUAUUCUCACCUCUCAUUCCUUUAUAGAGAAGAGCAAUGGAAAGA | |
| | AGAAGGGAGCAGGGAAACAUGGUCAAGGAAGGAAAGUGAUUCUCAAUCAG | |
| | GAGAAAAGGUAGCUCUCACUGGUGAAAAUUUGGUGCAGUACAACCAAAAU | |
| | ACAAAAUCCAGUUUACAAAAAAUGAAAUAUUUCAGUGACCCAUAUAUUUA | |
| | UUUUUCUGUGUUUUUCUGCAUGGGUUUGGGUUUUUUCCCUAUUAUUUCAG | |
| | AAUUGGGCUGUAGCCUGCAAGUAAAUUAAAGAACACUUGUUUUCAUUUUU | |
| | AAUUAUUGAUCAGCUGUCUCUAAGGUUCAUUUACUAUUAAUGCAGAGGUA | |
| | GAAUUGUGUUUUUUCCAUACUCUUUAGUUCUUGAUGAACCCCAUAUGGUU | |
| | CUGGUGCUCACAGCUCUUCAAUAAUUCAAGAUUAUCAUGGAAAAAUAAUC | |
| | AAGUUUUGAAAAUCUAGAUCAGUGGUUUUCAAAGUUUUUUCUCCUGUCAA | |
| | CAAUAGAAAUAUUUUUUUUCUAGUUAAAUUAUGAUCUGGACUCAAACUUU | |
| | UAAACUGUGUUUAAGUGGAGCUGCUUGGCUUUUGUAUAAGAAGGAAGGUU | |
| | UCUCCAUGACUCAAUUAACUUUCCAUCCUCUCCGGUGACCCCAGAGGUAG | |
| | CUCUUUCAAAGGUUGGGGCUCCUGUUGAACAGAAUUUGAAGUACACUGAG | |
| | AUAGAUGAAAAGCAGUUAGGAUGUAGCUCAGGCUGAGUUCUGAGAUAGAG | |
| | CUGAUCUGGGUUCAAACCAUGAACCUCACCAUCAACUAGGCUUGUUACCA | |
| | ACAGCAGGUAGCUGAACUUCUCUAAUAUCAGUUUCCUCCUCAGUAUAAGU | |
| | GGGGAGAAAAUAAUGCCCACCUCAUAAAACUGUUUUGAGGAUUGAAAUAA | |
| | UGCAUAAGAAGUAUUUGGCAUGUAUUGUUACUUGUGUCAGUUAAUAAUCU | |
| | CUUCUGAGACAUAUUCUAUAGUGAUACAAUGUUGGCAGUAUGAUGAAGCC | |
| | AUAGAGAUAAUGCUACUCAGAGUACAGACUGCUUUCUUGAUGACAGACCU | |
| | GAAUAUUUCAAAUUAUAAUCCUAGGAGGCUAUAGUUUAUGCUUUUUUCCA | |
| | ACUCUAAGAUUUGUAUAAAGACACAUAUAAAGUGUAUCCAUUUUGAAUAA | |
| | UACAUUUAAAGGGAUACUUUAUCCCCUAAGAUGCUGCACUGGUGAAAAUC | |
| | CCUAGGAUAAUGUGUAAUCUUCUUUGACAGUAUGUGCACGUAAAAUAGUU | |
| | UCACAAGUGCAAGAUGGCAAAUGUAAUCUUCCCAUGUGCAAAUAGACUUU | |
| | AUUCCAUUGAGGGUAGAGUCUCUACAGCUGAGUUUUUUAAUGCCAUGGUA | |
| | UCAUUUAUCUAAUUUUUUUUUUUUUUUUUUUUGAGAAGGAGUCUCGCUCU | |
| | GUCAGCCAGGCUGGAGUGCAGUGGCGCGAUCUCAGCUCACUGCAAGCUCC | |
| | GCCUCCCAGGUUCAAGCCAUUCUCCUGCCUCAGCCUCUUGAGCAGCUGGA | |
| | CUACAGGCGCCUGCCACCACGCCCGGCUAAUUUUUUGUAUUUUUAGUGGA | |
| | GACGGGGUUUCACCGUGUCAGCCAGGAUGGUCUCGAUCUUCUGACCUUGU | |
| | GAUCCACCCGCCUCAGCCUCCCAAAGUGCUGGGAUUACAGGCGUGAGCCA | |
| | CCACGCCCGGCCUCAUUUAUCUAAUUCUUAAUAAGCAAUCCAUUUUGCUU | |
| | UGUUUCUUCUCUCCCUCGCUUCCUUCUCUUCUUCCUUGCUUCUAUUUGGU | |
| | GCCAAACACCGCGUUUUAUUUAUGAUAUUAAAAAGUAAAGUGAGUGUGAU | |
| | GGUGGCUGGUCACCCAUUGUUUUGAAUUAAUAACAGAAAAAAAGAAAUAU | |
| | UUUAGUGGCCCAUAUGUUUAUUUUUAUGUGUUUAUGUAUGUGGUUUUGGG | |
| | GUUUUUUAGUGCCUUCUAUCCCUAGAAGUCACUUUAAUCUCAAGACUGUC | |
| | CAAAUAAACUUUUCAGCAAAAGCAUCAGGUGAGAAGUCUGGGAGAAACCA | |
| | UCCUAUAAAACAAUAUGAACAGAAUGGAGCAGUGAACACAAUGCUCCCAU | |
| | UCUUUCUCUGUCCUCCAAGCCAUCUUUCUUCUUAUAGACUCCCUGCCAUG | |
| | UGUUGCCUGGCUCAGGUUCUACAUGUUCAUCCUGCCAUAUUGGCACUGGC | |
| | UUCUUUGAAUUCUGCUCUAGUUCUGAGACGGUGGCGCUUUAUUCACUACU | |
| | AACUCCCAGGACCAGUGCAUGCUAUUCUUCAAUUUAUUGUGUAGAUUAAA | |
| | AGGUAUCUUCUUAAAAAAUGAUGCAUGCUGCUGGGAAAUACAGACACUUU | |
| | AAUUUUACUUAGAAUCUUACUUGGAAUGUGUUAGUAGCCAGAUAAACAAG | |
| | CAAGACUGACUCAGGAAAGAAGCCAGAGAAAGGGUAAGUGUGUCAAGCAA | |
| | AAGUAGACAGCAGCACCAAAUUGAAAAAAAAAAAAAUGAGAGGAGGAGAA | |
| | GAGGCUAAAGGUACAUCUCCUUUUACUUUUCUUUCUUUCUUUCUUUCUUU | |
| | UUUGUAAUUUUGCUUUAAGCUUUCCAUUCAAAUUAUCUUAUGCUUCUUGG | |
| | ACUAGCUAUUAAUAUUAGACUUAUGGUUGAUUAGCAUUUCAAAGAGAUAG | |
| | AGGCAAUGUAUGAUGAUUCUGAGUCACCUACUCCAUUGAAUGACAGCUUG | |
| | ACUAACCCAGUGACACAUACAGGUCUCAGCCACACCCCUAUCCUGAACGU | |
| | GUCUUCUCAGAAUUCUCUCCCUUAAUCUUGAUCUUUGUGUUCUUCUUUGA | |
| | CUUUCCAUUUUAUGUCUCUUUUGCCUCUUGUAAUGAAUUCUCUGUUUUCC | |
| | UAAUCUUCCUCCUUACCUCUUUCAAAGUCUGUGUAUUUCUUUCUCCCUCU | |
| | UUGUGAGUGAGCUUUCUCUCUCUCUUUUUCUCUAAUAUUUUGCAUACCCU | |
| | UAAUUCUUAUCUGUACAAGCCAUGGAUUUUGAAUUAUAUAUUUGAAAUAU | |
| | UAACUGCAGCUAUUUAAUAAAGAUUUACAACUCUAGGUUUCUGUUAAAUA | |
| | ACAAGUAGUUGAAAGUUAAAAAUUUCACAUUUUACAGGAGAGGUUUUUUG | |
| | CUAUGUAAGCCACUUCUUCUUUGGACUUUAGGUCAGAAUAUUUAAGUAGA | |
| | AAAAGAACUAUCAGUUCUAAUUUAAGCUGUUAUUUUAAUGUUAUAGACAG | |
| | UAAAUUUAAAUUAUUCAUGGCAUUAUGGAAUAUUCCUGGGUUUCCUGAUC | |
| | CUCUAGUUUUAGGCUACAGUGUUUCAUGAACUGGCUUAACUACAAAAACC | |
| | AUUAUCUAAAAUCCAUACCAAACAUUAAAAGGGCAGAAAAGGGGAGAAGG | |
| | CAUACAGACAUUGAAAAAUUCUGCACAAACUUCAGCCUGUAAGUGCUUUG | |
| | AACUCACACACAACCUUUAAUGUUUUCCUUGCAUUCUUUGAAUGAACCUC | |
| | ACUUUUCUGUUCAGAACCCUAGCAUGUCUAGGAACUUACAUUGCAUGUGC | |
| | UUAAUCAUGGAAGUGACCAUAUGUUCUUUUUAUUUCUAACUUUAUUUGGC | |
| | UCUGUCUGCCCUGACUUUUUCCUCUCUGGUCAUAAAGUUUUUAUCUUACC | |
| | AUUCAGUUUCUCCCAGAAAGCCUCUGAACCAGUCCAACUUUGUUAUUUCU | |
| | GUCUGCUCCCCGAGUCCUUGAUGGAAGUGUGGGUGAGGAUGGGGGACUGU | |
| | UUAAACUUUUCCCUUUUAGGUUACCAUCUUAUCUCCUUGCUCUCCCAGUA | |
| | UCAAUAUUCUCUCUGCUAGAGAGCAGCUUCUCAACCUACUUGGGUCCCUU | |
| | AGGGAUGUCUCUGCAUACUAAUCUUUCAGUACUUAUCUGCAAUCCAUUAC | |
| | UUACCCUCAUUAAAUACAGUUUGGAUCAUUUCCCAGAGUCAAUGGCAAAA | |
| | AUAAAAAGGAAUUUUUAGUUUAAUUAAAGACUGGCACCAUUUUUUAAUUA | |
| | AUUUUUUGACAAAUAGUAAUUGUGUAUAUUUAUGGGGUAUAAUGUGAUGU | |
| | UUUGACACAUGUUUAGAUUGUGGAAUGAUCAAAUCAGAGUGAUUAGCAUA | |
| | UCCAUUACCUCAAAUAUUUGUUAUUUCUCUGUGGUGAGAACACCUAAACU | |
| | CCUCUCUUUUAGCUAUUCUGAAAUAUCUGCUACAUUAUUAUUAACUAAAG | |
| | UCAUCUUCCUGUGCAAUGGAACACCAGAACUUAUUCCUCCUAUCUAACUA | |
| | UAACUUUGUACUUAUUGACCAACAUCUCCUCUUUUCCUGUUCACUUCCCA | |
| | ACUCCCAGCCUCUGGUAACCACCAUUCUCCUCUCUACUUCUAUGAGUUUA | |
| | ACUUUUUUAGUUUCCACAUAUAAGUGAGAUUAUACAGUGUUUGUCUCUCU | |
| | GUGUGUGGCUUAUUUCACUUAACAUAAUGUCCUCCAGGUUCAUCCAUGUU | |
| | GUUGCAAAUGACAGAAUUUCCUGUUUUUGUAAAGGCUGAAAAGUAUUCCA | |
| | UUGUGUGUGUAUAUAUAUGUAUAUGUAUAUACACACUUUAAAAUUAUUCA | |
| | UCAGUUUAUAGACAAAAGUUAUUUCCAUAUCUUGGCUAUAAUGAAUAUUG | |
| | CUGCAAUGAAUAUGGGAGUGUAGACAUCUCUUUGGCAUACUAAUUUUAAU | |
| | UCCUUUGUGUGUACACCCAGUAGUGGGGAUUGCUGGAUAAUAGGGUAAUU | |
| | GUAUUUUUACUUUUGUGAGGAACUUCCAUGCUAGUUUCCAAAGUGGCUGU | |
| | ACUAAUUUACAGUAGCAUCAAUAAUAUAAAGUCUUCCCCUUUUUUCCACA | |
| | UCCUUAACAACAUUUGUUAUCUUUCAUCUUGAUAGUAGCCACCGUAACAG | |
| | GGUGAAGUGAUAUGUCAUUGUGGUUUUAAUUUGCAUUUUACUAAUGACUA | |
| | GAGAUGUUAAGCACUGUGGGCAUUUGUAUGUCUUCUUUUGAGAAAUGUUU | |
| | AUUCAAAUUCCUUGCCCAUUUUUAAAGUUAUUUUUUUCCUUGUUAUUGAG | |
| | UAGUUUGAGUUCCCUGCAUAUUUUGGAUAUUAGCCCCUUAUCCAGCAUAU | |
| | GAUUUGCAAAUAUUUUCUCCUAAUCUGUGGGUUGUCUCUUUACUCCCUGA | |
| | AUUGUUUUCUUUGCUGUGCAGAGUUUUUUGGUUUGAUGCAAUCCCAUUUG | |
| | UCUAUUUUUGCUUUUAUUGCCUGUGCUUUUGGGGUCAUAUCCAAGAAAUU | |
| | UCUGCCCAGACCAAUAUGGGUACUCUCCCCUAAGUACUUUUUAGUAGUUU | |
| | UACAGUUUCCAGUCUUAUGUUUAAGUCCUUAAUCCAUUGUGAGUUUAUUC | |
| | UUGUAUUAGGGGUGAGAUAAGAGUCCAUUUUGGUUCUUCUGUAUGUGGAC | |
| | UUCUGUUUUCUCAACAGCAUUUAUUGAAGUGACUGUCUUUUUCUCAUUGU | |
| | GUGUCCCUAGUGCCCUUGUCAGAAAUCAAUUGACUAUUGAUGUGUGGGUU | |
| | UAUUUCUGUGCUCUCUGUCCUAUACCAUUAGUUGAGGUGUCUGUUUUUAC | |
| | GCCAAUAUCAUGCUGCUUUGCUGUUUUGAUUACUGUGGCUUUGUAUAUUA | |
| | UUUUGAAAUCCUGUAGCAUGACGACUCCAGGUUUGUCCUUUUUGGUCAAC | |
| | AUUGCUUUGGCUAUUUGGAGUCUUUUAUGAUUCCGUACAAAUUUUAUAAU | |
| | UGUUUUUUCUGGGCCAAGUGUGGUGUCUUAUGCCUGUAAUCCCAACACUU | |
| | UGGGGGGCCAACACAGGCAGAUCAUGAAGUCAGGAGUUUGAGACCAGCCU | |
| | GACCAACAUGGUGAAACCCCGUCUCUACUAAAAAUACAAAAAGUUCGCCG | |
| | GGUAUGGUGGCGUGACCUGUAGUCCCAACUACUUGGGAGGCUGAGGCAGG | |
| | AGAAUCACUUGAACCUGGGAGGUGGAGGUUGCAGUGAGCCAAGAUCACAC | |
| | CACUGCACUGGGUGACAGAGUGAGACUCUGCCUCAAAAAAAAAAAAAAAA | |
| | AAGAAAUAAUUGUUUUUUCUAUUUCUGUAAAGAAUGACAUUGGGAUUUUG | |
| | AUAAGGAUUGCAUUGAAACUGUAGGUCACUUUGGGUAAUAUGGACCUUUU | |
| | AAAAAUAUUAAUUCUUCCAGUCUAUAAACAUGGGAUAUCUUUUCAUUUGU | |
| | CAUCAAUGUUUUAUAGUUUUCAGUGUAGAGAUCUUUCACUUUAUUGGUUA | |
| | AAUGUAGUCCUAAAUAUUUUGAUUUUUUGAUGCUAUUAUAAAUAGGAUUG | |
| | UUUUCUUAUUUCUUUUUCAGACACUUUGCUGUUAGUGUGUAGACAUACCA | |
| | CUGAUUUUUGUAAGUUGAUUUAUGUAUCCUGUAACUUUACUGAAUUUGUU | |
| | UUUCAGUUCUAGUAGUUUUUUUUGGUGGAGUCAUUACGAUUUUCUUUAUA | |
| | UAAGGUCAUGUUGUCAGCAAAUGAAGAUGAUUUUACUUCAUCAUUUCCUA | |
| | UUUUGAUGCCUUUUAUUUCUUUCUCUUAUCUAAUUGUUCUGGCUAGUACU | |
| | UCGAGUGCUAUGUUGAAAAGAAGUGAUGAGAGUGGGCAUCCUUGUCUUUU | |
| | UCCUGAUCUUAAAGGAAAAGUCUUCAGCUUUUUACCAUUAAAUAUGAUGU | |
| | UAGGUGUGAAUAUUGGCAUCAUUUUCAACAAUAAAAAUGUUUUUAAAAUU | |
| | UUAUGUUGUGGCAAAAAUUGGCUAUGCAAGAUAUUUUUAUCAUUUUAUAA | |
| | AAGAUUAUGGAUCAAAAAAUAUUAAUGAAAAUAAAACAAAAUAUAUUAAA | |
| | GCUUCCUCUAUUAUCUUUAUUCAACUUAGGUUUAAAUUGUAUGAUAAAUU | |
| | AGAAAUGGAAGAUAUUAAGAAAAUAAAAGGCUUUAUAGAACUUUUGUAGA | |
| | AAUUUGAAAGAUUUGCAUAAUUUGUAUAGAGUAAAAUUGGGGACAGUUUU | |
| | CCAUGAGGUCAUAAGUACAGGAUAGGAGGGCAUUAAGGUGGUGGAAUAAA | |
| | AUUGGUACAUACUGGUAUGGGACAUAAUUACAGCAAAAUGUGUGCAGGGG | |
| | GUAGGAUUCAUUUAUUCAGUCAGUUGGACAGUCAAUUAUAUAUUUUACAA | |
| | AUUGUCUAGUUAGUGUUAUUUUGUGCCAGUCAUUAUACUAGGUUUUGGGA | |
| | UUUCAGUGGUAAAUAAAAACAGACAACAUGAAGUAUAACUGUAUAAGCAU | |
| | UCAUUGAGUAAUAACACAAUAAUUUAAUGAUUAUGUAUGUGUUUGGCAGG | |
| | AAGACAACCGAAGCCAUAGAACCACAUAGCAGGGGGUCUUGUUAUGCAGA | |
| | GUGUAGAAUCAGAGAUUUCCAUGAUGAAAUGAUAUUGAAUUAAAUAUUAA | |
| | AGUGGGAGUGAAGAGAACAUCCUUUCUGUUUUAUUUUGAGAUUGGCAACC | |
| | CUCGGCAUUCUUAAAGUUCAAGGGGAAGGAUUCAGUACCUAGGGAAUAGU | |
| | AGAACAUAUGGAGGAAAAAAAGAUAUCUGCAAGAUAUCUGAGAUGGUAGA | |
| | AAUAGGAAAUGAAGUUGUAGGAUUUUGUGGUGGGGAAGCGGGUCACAGUU | |
| | UAGUGCUUUCCUUAAGACAGGAAAAGAUACUUGUGAUUGUUUUGUAAAUA | |
| | GGCUCUUGAACAGGGAGGAAACUGGUGAAGCUAUUGCUUUAAGAAUGGUG | |
| | GCAAUAGAUUGUUGAGCGAUUGGAAUAGCUUAAGAGUGAAAGACCAGGUC | |
| | AAGUGGAAGAUAGCUGCAUCUAAGCAGUCAUUGAGUAAUAAGGUCAUGAA | |
| | UCGAGAUAGCUUAAUGUCCACGUGAAAAUUAUCUUCUAUGUUUUCUGUUU | |
| | UCGUGACAUGUUUUCACUCCUAUUUUUUUUAAAACAAAAUGCAGUUGUGU | |
| | GUAUCUCAGAAGUCCUAUUCAGUAAAAAUUUUCCCUAUACUCUCAGGAAG | |
| | AGUUUUAUUGCAACUCCCCCAGAAUCCCACAGAAUUUGAUUUCUACCUCU | |
| | UAGUGCACACUCUAUGAAACAAUGAAUUGUUUAUGUUCUUGUUACCUGAA | |
| | UAAAACUGAUAAUUUCUCAUGGGCUUGGAGAGUUGUUAUUUAUUUUUUAU | |
| | AGCAUACACAAGGCAGAAUUUGGAAAAUAUUGGACACACAAUAGAUAACA | |
| | AUUAGAUAUUGUUAUUGAUUGACUGAGUGCCUAGAUGAAGAGAAUCAACA | |
| | UUUGCAUUUUUUAUAUUUUCCACAGGUAGCCUGUCUUGUUGCUUUUCUUC | |
| | UUAGUGUAUCUUUAAGUAAAAGACUUCAUAAUUUGGAAAAUGCCAAUACC | |
| | UUUAUUGUUGAAAUCAAUUGCAUGCCUAAAGCAGACAGAUUAACUGCUUU | |
| | UCAAGACAUAGAGAAUUCAUGGUAACUGGUUUAUCUAACAGUAUUAUUAU | |
| | UCAGUUUCAGAAUAAUACAUACCAAAAUAUGUUGUGUUUUCCCAUUCUUA | |
| | GUGGCUGUUCUAACAGGUUCACCAACAACGUUGUUUACUUCUCUCUUUCU | |
| | CUCUCUUACUUUCUCUCAUGUCUCUAUAUAUCUAUAAUCUUUCUAUCUAU | |
| | CCACUGAUCUAUAAUCCCAUGUAUAUACAUAUAUGUACCCUGAAGCGAGA | |
| | UAAACAUGGAUUUAAAUCGUGUUGCCUUAAUUUAACUAUGACUCAGAAAU | |
| | GUCAUACAGUCACUGAGCCUCAGUUUCUGCAUCUGAAAAUGGUAAAUAAU | |
| | UUCUCUUAUCUUGCAGACUUUAAGACAGGAUUAGAAAUAACAUCUUGUGA | |
| | AGUAUUUAAUAAUUGGUAGACAAUUCACAGUGGCUAUUAUUUUGGUGUAU | |
| | ACUUUAUGGUACUCAUUGUUAUGUAGUUGUAUAUAGGCAUAAAUGGUUAA | |
| | UGCUAUUCAUAACUGACAAUGUAUUCUGAUCCUUUUUGGUGGACAACUGA | |
| | GAGAAAAGUCUGUGGUUUCAGAUUCUGCCCCUUUAGGAAUGGAUAAUUGA | |
| | GAGGAAGGAGCCGGGAUUCUUCAUUCCUCUCCCUCCAUUAGCAUCCAAAC | |
| | UGGAACCUUCAUGAUGGAGGAUAGGAUGGAUUCUCCUGUUAGAGAAGAUA | |
| | GUUGCACCAUCUGAACACUGGUUACUCAGGAUUCUAUGUUUAUCUUUUAA | |
| | CUUUUUCAAUGAGGACGACUAUUGUAUUGGCAGAAAGAGUGCUUUUUGAU | |
| | AUGGUUUAUGUAUAUGCCCAACUCCUAAUGCACAAACCUGUUAGUAGAGC | |
| | ACAUUCAGGAUACUCAAGCCCUGACUACUGUUGAAUAAAGAAACAAUAAC | |
| | CCUAUUUUAUUUAGAGGAACCACCUCAAAGUCUCUUGCAGUAAUGCUCAA | |
| | UUCUCUUCCCUUCCUAACUCACCAGGAAGAAGGGGGCUGGAGGCACAAAU | |
| | ACAUGUUUCUUCCCACUCCCAAAGAGAAAGGCUGUGCAGUGGGAGUAGGC | |
| | UAGAUGUUACAAUUCUUUGCUCCAUUAGGGGAUUAAAAUGACAGGUGACA | |
| | CUUCUCUUUUAUAAGCUAGGCACCUAGGAAAAAAAGGUUGCUUGUUUUGG | |
| | GUUUGCUUUAUAAUAAGAAGAAUAUGCUGAAGCACAGGGUGCUAGAAAAU | |
| | AAAAGAUGGGUAAGAGGUUUGAUGAAAGACUGGGAAUAAAAGUAGUUGCA | |
| | UUUCUAGAACAGCAUGAAAAUGUAAUCCUACAAUGUUUGAGAUUGCCAUG | |
| | GAUGAAGUUUUAAAGAAGUGACUUUGCUCAGGAAAUUAAGUUUUUGAUCA | |
| | AGGAGAGUAAAGGUUAAGAAAGUAUUUGCAAUUUGGUAUCAGGUUUAGAG | |
| | AAGACUUCAAGUGUUAUAGUAUAGAGGCCAUUUGGAAAGUCAACCCUUAG | |
| | CCUAAAGUAUGGAGAAAGGGGAGGGAGAGGAGGGCUUUUAAAAGGAAGUA | |
| | GGAAAGACAAAAUCGUAUCUUGCUCAUUUGAUAAUUUUGCCUAAAAUCUG | |
| | AUGGGACUGAUCUUAGCUUAGUGGCCCCAUCUUUUUAAAAACGUUUCAGC | |
| | CCAUCUUUUCUGGAAUUGGUAAGAUUGGAAGUGGAUAGCAGCUGGGCGUG | |
| | UCUUCAAAUGAUUUGUUGUUGAACAGAAUAAUUCCUUUUCUAGAACUAAC | |
| | CCAUACUAAGGUGUUGUAUAGAGGUGGAGUAGGGUGGUGAUAGCUAGUUA | |
| | UUUAUUGGGCAAACAUGCUAAGCAAAGUAGCUUAGAAAGUAUAAUGGCAC | |
| | AAAGAUAAACUGUUGGCUUACUUCCUGCUUAGUGAAAUAUUGUUUAUUAA | |
| | GACUCAAUUCAUAGUUGGCUUCUAUUGAUUUUUUACCAUCCAGUACUCAA | |
| | AGAUGUGCAAAAAUGUUCCAGCAUUUGCUCAAUAAUCAAAUGAUCACUUU | |
| | CUUUGUAUUGACAAUCUCUCUUGAUGUUAGUUUGUUCCACAUGUCAUCCA | |
| | AGGCUGACAAUCGUUAUAGUAGCCUCAAGUUUUUCGUCAUUACAACUUCU | |
| | UGACCUCUCUCCUUCUUUCUCUUUCUCCAACUUAAGACCCAGGUAUAUAG | |
| | AACCAUUUGCAACUUCUACAACAUACUGUCAUUGCUUAUACUUUACCCAG | |
| | UGGUUGGAACAUUCUAACUUACUUCUUCACUUGCCUCACUCUACUGAAUU | |
| | CUCAAGACGGGGCUCAAUGUCCUAUAGGAAGCAUGGCCAGUUGCAGGAUG | |
| | AAGUCAAAGAUAGCUGUCUCUGCUACGUGCUCCAGUUAGAUUUCUGUGCU | |
| | UUGUAACAGCAUUAAUCACAACAUAUAGCAUGUCAUUUUCCUGCUCCUCU | |
| | CUUUCUGGGUCAUGAAUAACUUGAGGAGGAAGAAUCUAUUUUACUUUUGC | |
| | UUUGAAAUACACACACACACACACACACACACACACAUACACACACACAC | |
| | ACACACAGAAUACCUAAUGUAUGAUUCAACAUAAUGUCUGGCUUAGUAGG | |
| | UCAUCAGUAGGUUCAAAGAAUAAAUGAAUAUAUGAAUUGCAAGUGCUUUG | |
| | CAUAAUGGAUAUGUAAUAAGUAACUGUUAAAUAAAUGUUUUCAAAGGAGU | |
| | UACUCUUCUUGAAGAAAGCAAACUUCUUGAUUCUAUUUUUAUUACAAAAG | |
| | UUUAAAGAUAUAUUUAAUGAGACAUUUUCAAAGGAAAUGCCAUAAAUGGA | |
| | GGUCAUUAAUCAUUCACUAAUUCCUUUUCAACCAACUUCAGGUGAUUGUU | |
| | UCCCCCAAAUCAAAUAAAUGUUAAAAUUAUUUGUAUAGCUGUUAAAGUGA | |
| | UUGAUAUAACUUACAUUUAAAUGGAAGAAAAGUGUUUUGAUAGUAACAUU | |
| | UUCAGCCCCAAGAUAGCAUGUUUUCCUCAUGUGUUUUCCAAAGAAAGGAA | |
| | GCUAGAUUACUAUGACUUAAUUUAUAAUAUAUUUCAACAUUGAUGGAUGA | |
| | AAAAAUAAUGAAGUGUUUAUUUACCUGAAGAAAGUAAAUUCAAGGUCCAA | |
| | UAUAUCUUAAUAUACUGAUUUAUUGCUUAUGUAAUGUAUAAUAUUAAUUA | |
| | UGUACAACUGCAUUAUACAACAUACAUAAUGGCAGUAAUUAUGUUCGUGU | |
| | GUCUGAAAUACAUUUUUGAAUGAGGUAUUUAUUUAUUGAAUUCAAAGGGC | |
| | AGAACAAUAAGUUAAAAGAAAGUAUAAGGCAUGGCCAGAGCUCGGUCCAG | |
| | UUUCCAGCAUACAACAAGCUCUCCAUAAAGCAUAAUUUCUCUUUUAAGGA | |
| | UAUUGUCCUUAAGGCAGUGUGCUGUGGUAGUGUUGAGUCAAGCAGUCUGG | |
| | ACUUAAUAAGACGUUUCCCUGGCAUUCAAAAGUUUUUGAUCUGUUAAAGG | |
| | UAGAAAAAUUUUUAUUAAAAAGACUAUUUUGUAUUAGCCAAAAGGGGAUA | |
| | UAAUCGUUGUUAAUGUAAAAAUAGAUAUGAUGUAUUACUUAAAAAUUUUU | |
| | GACUCUCUAAUAUCUUUCAAAUUUCCAACACCAACAGUAAUAUAAGUCCA | |
| | GUAAGCCAUGAAUCAAAUUGGAACUGUACUUCAGCAAUGGUGUAGUUCUU | |
| | UGCAGUGCAGUCUAUUAUGUUAAGUAGUUUCUUACCGUAUGAAAUUCAUG | |
| | UGUUCUUUUCAUAAACCUAGGAUUAUAAAAAGUCUAAUGUGAUGUGUGAG | |
| | UUUAAGGCUAUGAAUCCAGCUUUAGGGAGGUCUCAUUAAGGAAAAAGGGG | |
| | ACAGAUGGAUCCAUGUGCCUACUGUGUGUUAUAGUGCCUACUGAGUGCUU | |
| | UUGGAAUAAAAAGAUGAGUAAGUUUGAGCCUUUUAACAAUAGCUACUUCC | |
| | CUUAGAAGGAGGAAAGGAAAUUACAGAAUACCUCUUUGAAUUUUUGACCA | |
| | GUUAUAAAUUUUUUUUAAAAGUGAAAUAAAGAAGUUAACUAAUUGGAUUU | |
| | GCUUCACUAUUUCAGUCAAUCUGUGUUGAAUCUUUGAGCAAAACUGGUUG | |
| | AGCAGCUUACAUGACAGCUCUCCAAGUAAAUGGAAAAUUUCCCAAGCUCA | |
| | UGCUGGUUAGGAAACAGAAGUCCUCUUUUAAAGCAGUGCAGAACUCAGCA | |
| | GUUUGGGUUUCAUUGAACUGGCUUAGUGGUUUCAACUUAUAUAACUUCCA | |
| | UAUGGAAAGUUAAGGGGAACAUAGAAACCAAACCCUCUUAUUGUACCACC | |
| | UGAGAGCAGCUGCUCUUUCGGGGGUUUCUGGUACAUUGCCUAGGACCCUG | |
| | GAGAUCUUUUAUUUUUCCUAAAAAUCUGGAAUUCUGUCCAAAGACCAACG | |
| | AAACCAUGAAAGUCAAACUUAUGCAAACUAAUUUCUAAUGGCAGUGAGAG | |
| | GAGGAAAUCCAGUUUAAUCUCCUUUUUAAAGUGCACAAACACUUGGAACU | |
| | GUUUGACCUAACAGUCAGCAAAGGGUCCAGAGAGAACCAACACAAAACAG | |
| | GCAACAGGAUUGUGUUCUCUAGUAAAUCAUGGAUGGUGAAAAAAUUUUGU | |
| | UUUUCUUUUGAAAUAGUCUAGAGGCAAAUGAGAAUAUUAGGCUGAACACU | |
| | GUAAAUUACAGACAACUAGGAACCAUGAUUGGUAAGGACUAAAGAUGCAA | |
| | UUUUGAAACUAAAACAAACAAAAAAUUUAUGUCUCACUUGGUUAUGGGAG | |
| | GACAUUGAGAAUCAAUGAAGGUCUGUCAAGUAUUAUACUAAAUGUAUUAU | |
| | ACUAAAUGUAUUAUAUGUGCUUUGUCAUCUAAUUUUUUAUCACUUCUGUG | |
| | GGUAGGAAUUAUUUCAACCUAAUUUUUGCUUCUGAAGAAACUGACAGAGA | |
| | GACGGAAUGUGUUUCUAAAGGACUGAAAAGGGGUGAAUUUAGGAUCGAAU | |
| | CCUAGGUGUGUUUCUACCUGUUUUAAACAUACUGCUUUCUAUUUUGAGGU | |
| | GUUUAUAAUAUUUCUUUUAGGUAUAUGAAUUUACAAAUGCCAAGAGUGGG | |
| | UUGAACCAUUCUUAAAAUAUUGAUAGUUUCAUAAAAAAUAUUGUAAAAAU | |
| | CUUUACAUGCCUAAAAUUGAUUACUUCUCUGAGAAAGUUUCUACAUGAUU | |
| | GAAUUUGUGUGGCCGAAUGAAAAGACAUAUGUUUUAGAAGAGAAAAGUAA | |
| | UGAAUGAUUGAUGAUUGUGCAGUGUUUCUAUCCAGUGAUUUAGAACUCAG | |
| | CUUGUUUGAAUUGCUUUAGCAGAGGGAUCACAAGCGAUAUAUAUUUUUUA | |
| | AGUAAAUGUUUAAAACAUUGGCUAUAUCUUUAAUUAGAGAAAAAUUUCUG | |
| | GACUCACGACGAGCACAGAAAUUUGAGAAAAUUAGUGUAAUUAAAACUCA | |
| | GAUGUCCAGCAAGUUUGUAUUAUAUUUCUUUUGUGCUUCAUAAGAUGUGA | |
| | AUAAUAUUUUCCAAAUAAUUAAGACUGAAUGUUAACUCUGUGAGAAAAAG | |
| | AAUUUAUUUUUAGAAAUUUGAAUAAGAAUCUCCCUUUAAAAUCCACAUUU | |
| | CCAUGCCAUAUGUGAUCAUUUUAUGAGUAAAAUUUUACCUUUCCUAGAAU | |
| | CCAAGAUCAUUAUUAUAAACAUCAGCUACUGAGUUACUCUGUUAACUGCC | |
| | AUUGAUACAAUUUUUACAAAAUAUAAACUUGAGUAACAACUGGUCCUAGA | |
| | UUUAACAAACAGAAACAAAAAUUACCCAUUUAUUGUAAUAAUGACAUAUU | |
| | UUUAAAUCUUCUAUAACUGGAAUAAAAUAACCAGCCACUAUAUGUAUCUG | |
| | AUCCUCUGCAUUCUCAUUCCAACUGCAUGAUGAAGGUCAAACUCCUGCUC | |
| | AAUGCAUGAUCUUUGUCACGAGGUGGAGCUUACCAGGACUUAUGACUGUU | |
| | UACAAACUGACCUUUUUCUCCUUUGACCCCUAUCUUUUUCUUAGUAUAUG | |
| | UGCUGCUGAAGUAAGCAUUCUCCUUUGACUCUUUCCCUUCAUUUUAUUAU | |
| | UAUUUUUUUCAAUUUUUCUUUUCAAUUUUUUGUGGGUACUCCCUUCAUUU | |
| | UAGAAUCUCAAAGUAGCACUUAGUCACCAAAUGUCUGUUUCUUCUGUUUU | |
| | GAAGUCUAAUCUAACUCCUCUGGCUCCCCUGAGCUAAUGCUGAUUCCCAA | |
| | AGAGAAGGCACCCUUAACUCAAAGUUAGAAAUAUAAUCAAAGUCCCUAAG | |
| | UUUCCUCAAAUAUCUCUUUCUAGCAUGUCUUUCUGAACUGAGUCUUAUCA | |
| | GAAAUUUCUCCUGCAAAAAUACUUUCCUGGCUUGUACUUCCUAAUUUUAU | |
| | UCAAGAAUGUAGCAGGAAAUUCAUGAAGCAUUGAUGAAGUGUCUAUUAUA | |
| | UGGAAGAUACUCAGGUUCUUCACCACAAAGAUGGCUAAGACGCAUGCUCA | |
| | UCCCCCAAGAUGCCCAUGUUAAAGUGGGGGGCAGGGACAUAAAGAUGUAA | |
| | UAUAUAUAUAGAUGAUUGUGAAAUGAUAUAGACGAGAUUCAAUCAAAGAA | |
| | AAGAUGUUCUUGAAAAGCAAAUCUCCCACCAUGAUUAAGAGUCUGACUAA | |
| | GAAUAUGGGAAUGAACGUGAUAGGCAGUGGGCUGAGAGAUGUGCAAAUAA | |
| | AUGCUGUGUUCUAGGAACCAUGUAUAGUUAAAUAAGGCUGAGCAUAGACA | |
| | UGAGAGAGAUGGGGAGUGGAAGGAGGGCAGGCUGUAAGGGUGAGAACCAU | |
| | ACCACAAAUGAGACCAUGCUAGGGAAUCAGACUUGAUUUUACACAUGAUG | |
| | GGCUGCUUUUGGAGUGUGUUUAUUUUAGAUGAAUUAUUCUGUAGCAACAG | |
| | UGCAGAAGAGAUAUUUGAAAGGGGCAACAGUGGAGGCAGGAAAACCAGUU | |
| | AGGAAGUGAUUGUUGUCAUCUGAGCUGGAUAGGAUGAAGCUUUAGAUUGG | |
| | GUCCAUAGCGAUAGAGAUGGAGGAGAGAAAAUUUAGAGAAAUAUUUAUUA | |
| | AAUGUACGUGAUAUGGAGAGUUCUAAAUGUUUACCUUGAGCAGCAGGAAC | |
| | AGAUGCAGAUUUUGUGGGGCCUGAAAAUAAAGUAAAAUUAUUAAUUCAAA | |
| | AGCAGGUAUGAAAGUAAAUAUUUAUUUAAAAUGAGAGAAUAAAUCACAAG | |
| | GUUACACACUUUAAUAAAAUGACAAGCCACAAACCUCACAAAAUUGAGAA | |
| | AAAGAAAUGUAUUUUAUAUAAGCACAAACUCAGUAUUUCUUCAGUUCAAC | |
| | UUUCCUUUAGCCAUAUUCCAAAACUAUCUGUACUCAUUCUAAUAUCUCCC | |
| | AACGUGAAGAAAUAUGAUAGAGAUGAAAUCAGAAUUCAAAGAGAAUCAGG | |
| | AGUGAGCAAGAUACAUCCCAGAACCUUUGUUCCUGGUGGUAAAUUAAGCA | |
| | AUCAUGUCUGGCGAAAGUCAGCACUUGUUAUUUCUUAUCUGCGUAAAAUA | |
| | UUUUCCAUUUUAAACCAAGAAGACUCUGAAUUAUCGGGAAUUACAUGUUU | |
| | UUGGCCAUAAGAGUGGAAUAAUGACUGUAAUCAUUAACAGUAAGGAGUGA | |
| | GGGAGGCUACUCAAUGAGGAAAGAAAUGUGUAACAUUGCUUCACUUAGGA | |
| | AUAUUAUCCCCCUCUGCUAUUCACUCUUUUAGUGCCUUCCUGAAUAGAAU | |
| | AUCCUCCAAUAUUCAGAUACAAUGGGCUCUAGCAUACUCUUUAAGAACCA | |
| | AAAUCUUGAAAUCUAUAAUGAAUCAAUGCAGAUAAACUUUUCUGAACUUU | |
| | AAUUAGAUUUUUAUCUAUUUGAUGUCUAUGCACAGAAACAAGAUAGCAAA | |
| | ACUAAGAAUAAAUAGGAUUUUAUAAAUUGUUUUAUUUAAAGAAUAGCAAA | |
| | UAUUAACUUUAGAGUAUUAAUUCCUGUGAUUUCCUUGAUAUUUUUUAAGA | |
| | CAAUCAGUGAUUCCUAUGAGAAAUGUGGCAAGACUGGUGAAACAUGCCAU | |
| | CUUUUAAGCUUUCUGUUCUUCCUUUGAGAGACAGCCCAGCAUAGGGAUUA | |
| | AUGCAGGAACCAUGGAGUUUGACUGAUUUAGUUGGACUCCAGGCUGCUUA | |
| | CUGGUUAUAUUCUACUUAACCUUUAUUUGCCUCAGUUUCUUCAUCUGUUA | |
| | AAUAGGGAUAAAAUGACACCUACCUUACUGGGUUAUUGAGUGGAUUAUAG | |
| | GUGUUAAUAUAUGAAAAUAUCUUAGAAGCAAUGCCUGGCUUCUAAGGUGG | |
| | CUGUUAUUGUUGUUAUCCAAAGAGAAGGAAACAUAAUUUUCCUUAGACUA | |
| | GCCUAGAAGUCCCUGUACAACUGAGUCAGUCAGAUCUCCAGACUUUAUUC | |
| | CUAGCUACUAUACCAGGUAUGCUGAGCUCCAGCCCAGUGUUGCUCAGUGU | |
| | GUUCCAUAUGGAUGUUCUGAGAUAAUUUGUUAUGCCCAGUAAGUGUAGAG | |
| | CUGAAUUAAAUAUUUUAGAGGUGGCAGAAAGUUCCAUAUAGUUUUUCAUG | |
| | CUUUAGACUCAUAACUCCAAGGCUAUAUGGAUGGUCUCUGAAGAUGAUGG | |
| | GGCAAUAUCCUUUCUAUCUGUUACCGUGAGCUUUCACUGAACAUGACCAU | |
| | AAUUGACUGUGCUAAGACUUUGGCCUUUCCUACUUUUGUGGUAUAAGUUC | |
| | AGACCCUUUGAAGAAGCAGUUCAUAAAUGCAAUUAAAUAAAUUUCACCCC | |
| | AACAUUAGCUGUCCUGUCCUGACAAGGGAAGUGUUGCUUGUUUACUUAGC | |
| | ACUUGUGUCUUGUUAAUACUGGUGAUGGCCAUCUGUGGGUAUGAAGAGCU | |
| | AAUGGCUGUUAAGUGACAGACGAAUCAUUUCCACAGAGCAUGCAUACACG | |
| | UUCUUUCUUUGGAUGGCGUCUACAGUUUGUCCUUGGCAGACCAUUGCUAU | |
| | UUUCAGACUUGCUUCCAGUAUCCUCUUGUAUGUGUUGCCUCACAGUAACU | |
| | UGUCACUUCACUUAAGCAGAAAGCUCACGGUUCUCUCAUUGCUGUACCUC | |
| | CUGUACUAGCCUCUGAAUGCAAUGUUUGGGUGACUGCCAUUAUCACUUCC | |
| | UUUGGCUUUUGAUCACUAAACUUCCAUUAUCCAGGCAUCACUUGUAUUUA | |
| | UGAGUAAACUUUUCCUUUUGCACCACCCCCAUACUCCAAGUCUUAUAGAU | |
| | GAGCCAUUUCCUGACAGCUAAGGAGCAAUCAACUCCACUGUCCUCCUACA | |
| | UAAUAAUGAAUAAUCUUUUCUUGCAGUCAAUAAAACAGGCUCCCACGAUC | |
| | AUAUCCUUUUAUGACUUGGGGAUGCUGGUGAAAAAUCUUGACCAGAUAUU | |
| | AUUUGAUUUGGGAGAAUAGAGCAAUGUAAAUAUGGCAUUCAAUAUGCUUU | |
| | AAAAUAAUUAUCUGGAAGGUGUAACUUUCAUGCUAAACAUGAUUUACACU | |
| | ACAAAGAAAGUAGUCUAGGAGAAAUAAACCACGGAACUUGUGGGCAUUUU | |
| | GUAAUUGUCUAAUCUAGCUCCUUUAUUUUAUAGUUGUGGAUGCUGGGGCC | |
| | AAGUAGAGUUGACUUUCUCAAGGGCACAUAGUCAAGGAGCAGUGGAGAUG | |
| | CUGGAAUAGAGUCUUCUGAUUCACAGCCUAUUACUCUUCAUUCUCGGAUG | |
| | UUUUAUAACUGAUAAUAUCUUUGAAAAUAUCUAAAUAUUUUUGUUUGGGA | |
| | GGCUACUUGAGAUCACUUCCUUUUCACUGAUAGGUCAGAGGACACACAAG | |
| | AAGUCCACUCUUAACAUAUCAGUAAGUGAGUGAUGGAUGAAGUGAAGAGG | |
| | UAAGUGUAGACACCUCCAUUUUUAAGUUACAUUGUUGGGGAGAUGGGAAG | |
| | AAAUGCUUAAUGAAUUAGCUCUCCGUCUUUAGUCAAUCUAUCUACUAUGA | |
| | UUUAUCCAGUAAUGCAUUGGCCCCCGGAACUUGGUGCUUUUACAUCUAGC | |
| | ACAUCUCCAUAUCUUAUAUUUGAGUAAACUUUUAAAUAUUUAUUAAUUGU | |
| | UAUUCUGAGAGUGGAAAUAUCGUAAGUGUAGAAUAAGGAGAUUAGGCUCU | |
| | ACUCUAAAUUCUAUUGUGAACCUGAAUCCUAAUCAUGACAAAGUCACUAC | |
| | UCUUUGCCUCAGUUUCUUCACAUGUAAAAUUGAGGUGCUGCUUUAAAUAG | |
| | CAUCAUCUAAAAUUCAUUUCAGAUAUUGUGAAUGGAAAAGCUACAAUCUC | |
| | UGAAUGUUACAACUGUCAGGUCAGUUACCCUCAUGGAUUUCAUACAUUUC | |
| | CCAUGCCUGGUUGUUGGCAGAGGGAAAAGAGUGGGAGUGUGGUGUCUGUG | |
| | UUAGUAUUGCCAGCUGAUGGGCUUUAAAACAGGAGUAGUAUGGUUUGAGU | |
| | AACAAAAAUACCUUCUUAGGAUGCCUCACCAAAUAAUAGAGGGUGCUGAA | |
| | GAGAGUAUCUAAGUAAGAGACCUGGCUAUUUCUGGAGGUUUUUCAACUAG | |
| | ACCUACGUUUAAUAUUUAAAAACAUGAAAACUCAUGUAAUUAACAUGUAA | |
| | CUUAUUUGUAUUAAAAUUAGUAUGCUGACAUGAUAUUUUCUAAAACUGGG | |
| | CCACAUAUGCAUGGAUUUCCUAAAUAAUCCUUCAAGAGAAUAGAGCAGGA | |
| | AAUGAAGUGAUAUGCCCAUUUGCAGUGUGGUAUGGGGCUUCCUGAAUGUA | |
| | UAAAUGUGGAAGGCAGCAAGGUGGGGUUGGAUACCAGUAAUGCCAAGAAC | |
| | UUAAUUUCAGCAUUUCAACCACCGUGUACAUCCUCAAAAUGCCUUUCACU | |
| | UUAAUUUUGUAUAAAUACGAAUUGGAUCCCGGGUUACCUUUUGCUUUAGA | |
| | GAUGAAAAAAAUGUAUUUACGUUUCCCACACAACUGCUAGGAAUUAAAGU | |
| | AGUGUGAAAACUCCACACAAAUAAAUAAUACAUUUAAAACUAUUUUUAGA | |
| | AGAAUAAUUCAAAAAGACAGUAGUUUUGGGACAUAGGCAACAAAAAGUCC | |
| | CUUUUACCUAGGUUUCUUUGAUUCAGAGAUUCAGCAUACGGAUUUAUGGA | |
| | GACAGAUUAUAUCCUCAACAAAUUAAAGAAUACAGGAUAUACACUGUUGU | |
| | CUAAUAUAAUAUGAGGAAACUUUAAAAUUUAUUUCCAUGAAUUCUAUGUU | |
| | CAAUUGGAAAUAUAUGUUCAAUUGAGUAUCAGUAGAAAAAUCUAUUGUUU | |
| | UUACUAAUGACUCAGCUGCUUAAAUGAUCUAUUGUAUCGGUGGCCAGUGG | |
| | CAGGAAGGAGGCUCUGUGCAGUAUUGUUGUAACCAUGACAUUAGUGACAU | |
| | CAUUUAGCAGAAAUAGAAUUUGGGACAUUUGGAAAACAAAGCAGUGGCCU | |
| | CUUGGCUCAACUUUGUGAGAACCUAAUAAAUUCUAAGAAAGUAUAAUGGA | |
| | CAGUGUUGAUUUCUCCAUAUAUAUUUAGUGAUUUUACAUGUAUGCACACA | |
| | CACACACCUACAUACAUAAAGUAUUUCAUUUAAAUUUACCUACUAAUGUA | |
| | UGAUAUUCGCAAUGCCGUAUAGACUUUGGGAAGAAUGUUAAUCUUGUAUU | |
| | GCAAAGCCAUGCUCUACUUUAAAAGCAAUGCCUCCAGAUGUACAGCAUAC | |
| | UUAACAGGGCCAAGAAAACA | |
| 2064 | CAUACAUUUUUAAAAGAUUAAUAAAAAUAAGUCAGGUCAUUUUUUACCCA | RNA |
| | GUUAUUCCAGUUCAGGGUCUUGGGUGACCAGAGCCUGUCCUGGGAGCUCA | sequence |
| | GGGUGCAAGGUGGGAACUAACUCUGGACAGGACGUCAUCCUAUCGCAGGG | of |
| | UGCACUCACACACCCGCCCACACCCACUCAGACUGGGGCCAUUGAGACAA | SHAR |
| | GCCAGUUCACCUAGCAUGCACAGCUUCAGAAUGUGUGAGGCAACGGGAGU | ED_ |
| | GCAUGGACAGAAGCCAUGCAGACAUGGGGAGAACGUGCACACUCCACACA | 00113753 |
| | GACAGUGGCCCCGGCAGGGAAUCAAUAUUUUUUCUUGUCAAUGUUGCAAG | |
| | AAAAUAAUGUUGAAUGACAGGAAAAACAUUAUUCUAGGACCUACUGUAUU | |
| | UCUUUAUAAGUUUCUCUGAACAAACUUUGCUAGAUCAAGUUAUAUAUUUU | |
| | UUUAAGACCUUUGAUACAUAGUGCCAAAUUGCCCUCCCAUAGAAAAGGUG | |
| | UACUUGUUUAGUCAAUCUCACUGAUUUUGUAAGAGUGUGGUUUUUUCCUC | |
| | CUACUCUUACAAAUGCUGGGUGUAAGCCUUUUUUCCCAAUUGAAAGAUAA | |
| | AAAUUGGUACCUCUGUAUUUCAUUUUCCUUCGUUAAUAACAAGGUUGAAU | |
| | AUGUUUCUGUAUGCUUACAGCUAUUUAUAUUUUUUCUUUUUUGAACUGUC | |
| | UGUUUUUUCUCGCGUCCAUUUUUCUAUUGGGAUUUUUCAUAUUUUCCUUC | |
| | CUGAUUUGUAGGAACUCAUUACAUACAAAGAUGAGAUCUUCUGGGCUGUA | |
| | UUUCAGAAGUUUUAUUGUCCCGUGAGAUGUUAAUAGGUGAACUGAGGGGA | |
| | AAAAAGACUUCCAUGGUCAGAUUUAACACAUCAUUUCCUAAACUUCAUUG | |
| | ACCGUGAGGACUUUUUUCUUUUUCCUCCCAGAACACCCAUUGGCAUGGUU | |
| | CAGGACAUUGGUGUUCUGAGCACUAACAUUUGAGAAAUUGUUCUGUAAAG | |
| | GAUAUUAAUGCUUUAUCUGUUACAUAUGUGGUGGGAUUCCCCCUCCUCCG | |
| | CCAGUCAUUUGUCCUUUCCUCUACAUGAUUUUUUUUCAUACAGCUUUUAA | |
| | UUCGUGUAUUCACAUCUCUCAAUAUUUUCUUUUGUGGUAAUAUAGAAGGA | |
| | GGAACCCUGAAGUCAUACUUGUUUGCUUUGAAUCCUGGCACUGCCACUUG | |
| | UUGGGGCCUUUGGAAAAUUACCCUGUCUGUGCCUCAAUUUUACCAUGUAG | |
| | AAAAUGAGAAAAAUUAAUGUUACCAAACAAAGUGAUUGUGAGGAUUAAAU | |
| | UACUUGAUAGAAGUAAAGCACAGAGUAUUAGAUGUUGAUGAUGAUGAUGG | |
| | UGGUUUACUUUUUGGAUUAUAUAGAAAGUCUCUCUCCUUCUCUGUUGUAA | |
| | AAAAAAAAAAAAUUGCCCAUAUUUUAUUGUCAUAUUUUUACGGAAGUUAA | |
| | UUUUGAUAUGAGCUAUGGACUAAAUAAUUUUCUGUUUUCCCCAAUGCUUA | |
| | GCCAGAUUCUCCCACUGCAAUUAAUUGAUUAAUGCUCCCCUUAUCAUAUA | |
| | CUAUUACUUUCUUUUUCUGGGUCUUUCUCUUCUGCUCAGUCUAUUCUGAU | |
| | ACCAAGGCCCCUUCUUUCCUAUUCCUCUUUAGAACGUUCACAGCUACAUU | |
| | UGCCUAAAUCAAGGGCACAUGUUGGAUUCCCUCUGGGAGUUGCCCCAACA | |
| | GACCUUUGGUAGGGGAGAGGGGCCUGCAGGGUGGGAUCCAGGUGUCUCUG | |
| | ACACAGGUGAGAAAUGCGGAUCCUACAGACUGAAUUUUAACUGACGCUGU | |
| | GCCCUGUUCAUCCCCAUGUGCUGCUGAGGCUUGGUGUUGAGGGACAGCCU | |
| | GGGGACUCUCUUAGGAACAUUUAAACUCCAGUGUUGUUCAUGUUGUUCAC | |
| | CAAAUCACAGAAAAAGUGAGAUCCUCAUACUCUACCUUGUCCCUCCUCAU | |
| | UCUGCCGCGCCAGUCAAAAUGACCUCUAAAUAAGCAGAAAAGAUUUGAUU | |
| | AGGGGCAGAGGGGAUUCUAACCUGACACAUCCUAAGCUAUGGAAAGAGAG | |
| | CCAACUCGUCUCAGUUGAUAGGAAGAUUUGAUACUGGGACGCUCCAUGAA | |
| | GGUGAUACUGGAGCACCAUUGAGGAUGUUUCACUCAGAACUGAGAGUCCU | |
| | UGGGUUGCCUGUCAGGCCCAGCACAACCUUCUCCUUUUCUGCACCACAUC | |
| | AUCUCUUGCUCACUUGGUUCCAACCACAAUGGUCUCUUUGCUGUUCCUCA | |
| | AACAUGCUCCCACUUUAGGACAUUUGCACGUGGUUUUUCCUCCAGAAUGU | |
| | UCCUCCCCACAUACCCACAUAGCUUGUUCCCAUGGAAGCUUAAGGCUUUU | |
| | GUUCCAAUGUCACCUUGUCAGUGAGGCCAUCCCUGACCACUCUAUUGAAA | |
| | GCUGUGCCUGGAGAUCCUCAACCUUUCUUGCCUUAUUUUUCUCCACAGUG | |
| | CCAGUCACUGUCUGACCAUCUAUUUCUCCUCAUUGUAUUGUUGAUUGUCU | |
| | CUCUCCCCUAGAAUGUUAGCUUCGAAGAGUCUACCUUCUGUUUGGUUUCC | |
| | UGCUGUACUCUCAACACCUAGAAAAGUGCCUGGCAUAUAUAGGACUCCAC | |
| | UAUUGUUUGUAGAUAGAAGAGAAAAUGAGUGUAUCCGUCAGGCAAAGUCA | |
| | CUUGCAUGAUACUUCAUGAUGUGUCCAGGCCUAGUAACAAUAGCAUGUUU | |
| | UCUGAGAAUGUAGGCUGACUUAAAAGACUCUGGAUUUUCAAGCUAGCCUC | |
| | UACAAAUAAGAUAUCUCUAAAGUGGGGAGAAAGGCUGCUGAUAUGGUUUG | |
| | GCUGUGUUCCCACCCAAAUCUCAUCUUGAAUUGUAGCUCCCAUAAUUCCC | |
| | ACGUGUCAUGGGAGGGACCAAGUGGGAGGUAACUGAAUCAUGGGGGCAGG | |
| | GUUUUCCUGUGCUGUUCUCAUGAUAGUGAAUAAGCCUCUGACGGUUUUAU | |
| | AAAGGGCAUUUCCCCUGUACACGCUCUCUUGCCUGCUGCCAUGUAAGAUG | |
| | UCCCUUUGCUCUCCCUUCCUCCAUCUUCUGCCAUGAUUGUGAGGCCUCCC | |
| | CAGCCAUGUGGAACUGUGAGUCCAUUAAACCUCUCUCCUUUAUAAAUUAC | |
| | CCAGUCUCAGUUAUGUCUUUAUUAGCAGCAUGAGAACAGACUAAUAUAGC | |
| | CGCAUCUCUCAAAAGCGAGAACAGACAUAUGUCUGCGGUAACAUCUUUUA | |
| | UCUGAUGCCCUAGCUUCAUUCCUGAGCUGCCACAGAGCCACUCAAAAUGA | |
| | AAUUCAAAUUCAAACCACAGUGAGAUACCACCACACACCCACCAGUAUGG | |
| | CUACAAUAAAAAUGCAGAAAAUACCAAGUGUGGGCGAGGAUGGGGAGGAA | |
| | CUGAAACUCUCAUACACUUUGAAAAACUAUUUGGCAGUAUCUUCCAAAGC | |
| | UGCAUGUGCAUAUAUCAUGACCCAGCAUUUUCACUUCUGCAUACCACCCA | |
| | ACAGAAACACCUACCUAUGUCCAGCUGGAAGACAGGCAGGCACAACAUUC | |
| | ACAACAGCACUGUUUGUAAUGGCCAAAAUCUGGAGCAAAAAAUGGAGAAA | |
| | AAUAGUAAAAUGGAUUUUUUAUGUGGUAUAUUCACACAUACACUACACAA | |
| | UAUAUGAAAUGCAUGAUCUAUGACCACAUGCAAUGAUAUAGAUGUAUUGA | |
| | ACAAACAUAAUGUUGCACCAAAGUCAGACACAAAAUUGUAUAAACUAUGA | |
| | AUUUGCUUCCAUCAGUAUAAGAUCUAUAAACAGGUAAAGCUAUCCAUGCU | |
| | GCUAGAAAUCCAGAGUGACUAGAAAGGAGGAUUAAGGAAGCUUUGGGAUG | |
| | CUGGUAAUAUUCUGCUUCUUUACUGGGGUUCUGGUUACACAGGAAUAUUU | |
| | AGUUUCUGAUGAUCCAUCAAGUUGUACAUGUACAUGUGAAAAUUCCUGGG | |
| | UAUAUAAAUGUUUUGAAAACUAGGUCCAUGUAUCAGAACCACCAAUUGGA | |
| | GUUGCAGGCCACACAUUUGCUAAAGCUGUUUGUUACAAAAAGCUGAAACU | |
| | AUUCCAGGAUGCACUGCUCCAGCACUUGGGGUGGAGGUGGGUGGGAGGAG | |
| | GAGGUGGGGGUUGGGGAGGGAACCUUCUGACCCUGGCUGGGUCACACCUC | |
| | UGAGAUGCUACAAGAACGAAGAAUGGUAGGGGCCAGGUGUAGAUGGGAGA | |
| | AGGCAGCUGGUAGACAGCCUCGGCUUUCUGAGGUAUCUGAGGUCUCUGAU | |
| | GGCCUCAAGGCAAACACUGCCUCCUGUCAGGCUCAGAACAGUAAAGACUC | |
| | UCCAGCCAGCCUUCCUGAAUGCCAACAGCCACAGGACUGUGCAGGUCUAA | |
| | UUUCCAAUCAGAUAUGAAGGGAAGGAGAAUGAUCUCUGCUGAUAAACCGA | |
| | GUCUGGAAACCUUGUUAUAGAAUGCAGUAGAAAAAAAGACAGGAGAGAGG | |
| | GAAAAACAUAUGAAGCGGUUCUUAGGUAAUUUCUAAGAAGGAAGCUGCAG | |
| | CAGGAACUCUGGCUACAUACCAGAGAUCGCCCUCUUAUUUCUUUCACUUA | |
| | AUAACAUUAAACAGGAAAAGAAGGAAUGGAAAUUCAGGAACUUCAAGGUC | |
| | CCACUAGGGCACUGAGGACCAGUGUAGAGGAGGACUCAUCCUGCACCAGA | |
| | CCUAUAGGCAUGGAUUAUACCAUGCCCAUGGUGCCAAUUAAGCUUUAAGA | |
| | AUCACACUUGCUAGCAUUGGAGAGGUUAGGGGGUCGGGGAGAGGUGGUAC | |
| | CUAAAGAAGGAAAAAGACGACUAUCUGUGUUAGGCCAUUCUUGCAUUGCU | |
| | AUAAAGAAAUACCUGAGACUGGGUGAUUUAUUUAAAAAAAAAAAAAAAAA | |
| | GAGGUUUAAUUGGCUCACGAUUCUGCAGGCUUUACAGGAAGCAUGGUGCU | |
| | GGCCUCUGCUCAGCUUCUAAGAGGUUUCAGGCAACUUACAAUCAUGAUGG | |
| | AUGGUGAAGGGGGACCAGGCACAUCAUGUGGCAAAAGCAAGAGCAAGAGA | |
| | GAGAGAGGGUGCGGGGAGGGGAAGUACCACAUAUUUCUAAACAAUCAGAU | |
| | CUCGUGAGAACUCACUCACUAUGGUAAAGAUAGCACCAUGCCAUGAAGGA | |
| | UCCACCCCCAUGACCCAAACACCCCCCGCCAGGCCCCACCUCCAGCACUG | |
| | GGGAUACAAUUCAACAUGAGAUUUUGGCAGGGACAAGCAUGCAAACUAUA | |
| | UCACCAUCUGGUAACAUCCUAUGGCUAUGGAAGGGAAGGGAAUAGGCAAA | |
| | AUUGGUGGUUGAAUUUGGUUGACAACCAAAACUCAGUGGACAACCCCUUU | |
| | UUGGUAUCCCAGUGGUAGAAGCCACUUGGAGAAGGUUGGGAAGCUGAAUC | |
| | AGCCCAGGAUCUGUGUGGUCUUCAGCCUUCCAGGAAGUAGAUCAUGCUGG | |
| | ACAAAAACUUCAGACCUCAACCCACAGGAUCUAGAAAGGCCAGGAUGGAA | |
| | GAAGCCACAAGGAAAGGAGAUACUUAGAGCUCAGUGGUGCUGGUUAGGGA | |
| | GUCUUUAAGGACACUUGAGAGCCAGCACCUGUGCCCUCAUCCUGGCUUCA | |
| | GAUUCUGUGGCCUAAGUGCUAAAGCCUCCCGCCCACUCCUACAGAUGUGA | |
| | AUGAGAGUCAGGGAAAAACUGGGGGUGGGUAUGGGAGUCCGAUGAGAGAA | |
| | GAGCACAUCAAACCCAGGGUAUCUGUGAGAGGGAGCUGGAUCCAUAGGGC | |
| | CUGCUCUGCAUCUGCAAGUCCAGAUGUAAUGGCAACACACUUGGGCAGUU | |
| | ACCAUCGCUACCUAUGUGCAGAAGGCUACACAAACAGGAAGAGGAAAUAU | |
| | UCAAGCACUGGCUCGUUCAUUUUAACUCGAGAGUGUCUAAAACAGAUCCC | |
| | CAACAGCACGGAAAGUUUCAGUUUUGUAGUCAAGCAGAUAAAGAUGUGGC | |
| | UAAGGAGAGUAUUCUAAGGCAGAUGCCAAGUGUAUGCCCUCUGUGAGUGC | |
| | AAUGUGCAUUGCCCAUGGGCCUAGAGCAUUCUAGAAUAGAUAUGCAAAAU | |
| | ACAUGACCACUUAGGGAAAGUUGAGAUGAGUCACUCUUUCUAAGAGUAAC | |
| | CGUUUAAAACAUCACACUUAGUAUCUCUGAGCCCCACAGCACAUGCACUG | |
| | UGAAGAAUAAAAAUUCAGAUAACAUUUAGACUUGCAUGAACAAUUGUUUU | |
| | UUUUAAAUAGACACCAAAGGAAACUCGAACCCAAAUUCUAGAUUCAGAAU | |
| | GCCCAACACUGCUUACCAGAGACAAUGGAUUUUGGUUUAGGUUUUAAAGA | |
| | GUGAUAAGUAUCAGAUUCAGAUGCUAAGAGGUAAAAGGAGAAGACAGUUU | |
| | UGCUGCUCACUCAGUGGGUUUUCUCAGUUUUAGACAAAGGUCACAAGGGA | |
| | UCUUGGGGGCUCCAGACACUAUCGAGUAAUUGGUAAAAAGGAUGAUCAUG | |
| | AUAUCAAAUACCUGGAGUGCUAAUUUCUUUAGAAACCAAAUUAUAUAAGA | |
| | CCUUUGAGGCAUGUGAACGUUCAUCCCCAGUUCAGAACUGCCUGGAAGAA | |
| | GCAGAGUUGACAUUGUUCAUUUUUAAAUACUGUGCAAGUUCUGCUAGAUG | |
| | GCUAGCAUCAUAAUGGGGUAAGAGCAGAUCCUGAGUAGAUCUUCCCUAGC | |
| | ACUCCUUAGCUGUUUCCCAUGAACCUUAGAGAAUCAAAGACCUAUAGGCU | |
| | UAGUGGAUACUAAGAGAGAUUUGAAGAUAAACAUGCCAUUAAGGCAAAGC | |
| | CCUUCUCUGACCUGACCCAAGCAUGUCAGGCCAGAGACAGUAAGAAAAGG | |
| | CACCAGAAAAGGCAUUCCAUGGAUAAGAGAGAAGCCAGGGCCCUCCACCC | |
| | UUUUGAACAUUUUGAAAACUGAAAAGGGAAGGCCUGUAAGCAGAUGCUCA | |
| | GGGCCUUCAUGAUCUCUUUCAGCCAGGUUCUUGCACUAGUAUCAGCUGAU | |
| | CAAGUCCUUUCCCUCAGUUUAAACCAUGGACUACCGUACUGAGGGCUUAG | |
| | GAGCUGUGCUUUCUGACAUAGCAGGAAGAUAUCGGGAAGGUAGCUAAAUU | |
| | CUACUGGCAGAAGCUUAAACUAUGUGAGAUCUAAUAUCAAUAUCCUGUUU | |
| | ACAGAUCAGAGCCAUUGGCACAUAAUUAGGCAGUGAGACACCAAUUCUAG | |
| | GAGGUGUGUAUACAAGGUGGAGGGGUGAUCCACAGAAGCCUCCCCUCUGU | |
| | GCUCUCCAGAGCCAAGCAAGACAUGACAGGGAAGUAGAGGGUGGUCGUUU | |
| | CGGUUAUACAGAUGCUGCACCUCUUGAAGGAGUCAGAGGCAUGUGAGAUU | |
| | CCCAGGGUCCUAAGGGAUCUGAGUUCCUUUAUGCCAGAAAGCUUGGGCUU | |
| | UCCAAGGUAAGGCCAAUGGUAGCUCUCACUUAUGUGGCUCCGGUAAACCU | |
| | AACUGCUCCAGCCAGGAGGGACAAGAUGGAAGCUCAGCAUAGGAGGUGGU | |
| | CAAGGAAAGAAACCUAGGACCUCUGAGGUUGAACUAGACUAGUGGUUUUU | |
| | GUUUUUGUUUUUUUCAAGUAAAUUUGAAUUUGGCAUUGAGAUGGGCACAA | |
| | AUUAGAACAGCUUAAGGAUGUAUUAUUAAAAUGAGUGCUGAAAUAUUCUC | |
| | UAUGAAGACACUUUCCCCCAAGAAUACUGUGGUAUGGUUUUGAAGACCUC | |
| | AUAAGGCUUGGGAUGGAACAUAGUAUGUGAAUGUUGCAAUGCUUUCCACC | |
| | UUCUGGAAAUCUGUAACUGGAGGGCAGAAUUCUGGUGGAGGGUUCCACUU | |
| | GUAAGUCUCACCCUAAUAGUGUUGGUGGUACUUUAAAUAAUAAAUUGAAA | |
| | AAAAUUAUAUUAAUUUCUCUAAACUGAAAUUAUGAAACAUAUGUCACCAU | |
| | CCAUACCUGUGAGCAUUAUCCCUGUGCCUUUUCCUUGUCUCUCCUGGGGA | |
| | UCUCAGAGAGGCCUCCCUUGUUUUUCAAAUGAAUCAUAUAGCAGUUUAUU | |
| | CCUUGAUCUCUAGUUCCACAGUUCCUCUCCCCACUUCCCUCAGUAUCUUU | |
| | AUUUCCCUUCAAAUUUCACUUUACAGGUCUCUUCAUCUGAUCAGUGAGUU | |
| | UCUUGCCAAACAUAUUUCAAGACCUUCUGGAAGUACCUUGCCAAUAACCC | |
| | AUUGCCACAUCAUUAUUGCUAACUCUUCAUUAUUCUCCAUAUUCUCUUUU | |
| | CAAUUUCAAGGAUAACAAUAUCAAUCAGUGUGGACCUAACCUGUGCUUCU | |
| | UAAAGCCUCUCUGGACUGGGGUUUUAAAAGGAUCACACUAAACCCUUAAU | |
| | UACUCCUCUUUUCUUGUACUCCCUUCUCUGCUCUUGUCCUGGAAGAGAGA | |
| | UCAUGACAAGGGAUGAUAGCUCAUAGUGAAGGAGCCACUCCUUGGCUGGC | |
| | AAGAGGCUCUCCUAAACCACUACAAACAAUUAGUCUUAAUCCUGGCUUUC | |
| | AAAUAGAAGCACCUGGGGAGCUUUUAAAAAAAUAUGGAUGCCUGGGUCCU | |
| | AUCCCACACCAAUUAAAUCAGGAACUCUUGGGAUGAAGCCCUAGGGUUGG | |
| | UAUUUUUUAAAACUGCAGUGUGAUUCUAAUGUGCACUCAGUUUCAAAGCA | |
| | CUGUUAGGAGCUGUGGUUAGCCAUGAUCCUUUGUUGAGCAUCUAUUUGCA | |
| | GCCAGAUGUCAGCCCACACAAUCAGGGCAUAGAGGAGUAGCACAAGGAUU | |
| | CUCCAAUCAAGUUAUUCAAUUCUCCCACUCACUGCUUCUUGCCUGCUUUG | |
| | CCUCCCCCACUCUGAGCCAGGGCUCCUUUUAUUAAAGCAACUCCUAAGAA | |
| | CACAGGGACGCUCUCUACCCCUUGCAUGAACUCCUCUGCAAAUCUCUUAG | |
| | GAGUUAGCACUUCAAUCACUAUCAUUCAUUCAGAAAAUGUAAGUCACCUU | |
| | UUUUGCUUUGAGUUAAUCAUUUCCCAUUUUCCCAGGUGAUUCAGAAAGCC | |
| | AGUGCUCACUGACAAUGAGUGGGUAGACGCGUGGGCCUGCAGACCUGCCU | |
| | CGCUGUUCCAUUAACAGCAGAACACACUUCGAGAGGCAACUGACAAUGGA | |
| | GGCAACAACAACAACAACAAAAGCAGUAUUUUUCCAAUCAUAAAAAAAGA | |
| | AGUCUGUUUCUGAAAACACAGUUUGAUUUGUUUAUAUUUCAUGCAGUGUU | |
| | AAAAUGCUCAGUCGGUUCUAACCUUGUUUGCAUUAAUCCUCUUUUCGGAA | |
| | UUUGCACAUUUGUUUUCAAAGCAUUGGGUUGGAGCAGUUUGCUGCUACAU | |
| | GGAAAUCAUAUCAGCAAUCGUACUCAGAACAUGAAAUGAACCUGCACAUA | |
| | GAACCCACACAAUUUUAAUGUCUGCUAACCAACUGAUAAGCACAUAGGAA | |
| | GGGCAUACAAGAAUUAGAAUAGAGAGCCAUUGAGCCAACAAAGGUUCACU | |
| | UUCUAAGAAUCACCACCUUCUGAGGUUACUGGUUAGUACUCUCAACAGCC | |
| | AUUGCAGCAGAGGCUAAUUCAUUCAUUCACACAUUCAAGAAAUUUUUCCU | |
| | UGGACAUCUACUGUAUGCCAGGUACGAUGCUAUGAGUUAGGACUAGACUA | |
| | AGGAAUAAAACACAGUCUCAGCCCCCAGAGUCUACAGUCUUCAGCUAAUC | |
| | AUAUAAACAAAUAAUGAGCUUUGCAAAAGCUUCUCCUCUCAUUGGUAUGC | |
| | UCUUCUCCCAUCUCUUUGUCUGGCUAAAUUCUAUUCAACCUGCUAGAUCU | |
| | UGAGUAAGAUGUUUCUUUAUCUAAAAUAAGCGUCCCCAACCCCCAGGCCA | |
| | UGGACCAGCACCAGUCUGUGUCCUAUUAGAAACCAGGCUGCAUGGCUGGG | |
| | UGCAGUGGCUCACGCCUAUAAUCCCAGCACUUUGGGAGGCUGAGAUGGGU | |
| | GGAUCACCUGAGGUCAGGAGUUUGAGACCAGCCUGGCAAACAUGGUGAAA | |
| | CCCCAUCUCUACUAAAAAUACAAAAAUUAGCUGAGCGUGAUGGCAGGCAC | |
| | CUGUAAUCCUAGCUACCUGGGAGGCUGAGGCAGGAGAAUCACUUGAACCC | |
| | AGGAGGCAGCAGUUGCAGAGAUUGUGCCACUGCACUCCAGCCUGGGUGAC | |
| | AACAGCAAAACUCCAUCUCAAAAAAAAAAAAAAAAAAAAAAACAAGAGAA | |
| | GAAAAAAAACCAGGCUGCACAGCAGGUGAGCAGCAGGCCAGUGAGCAUUA | |
| | CCACCUGAGCUCUGCCUCCUGUCAGAUUAGCAGUGGCAUUAGAUUCUCAC | |
| | AAGAAUGUGAACCCUAUUGUGAACUGUGCAUGCGAGGGGUCUAGGUUGCA | |
| | CGUUCCUUAUGAGAAUCUAAUGCCUGAUGAUCUGAGGUGAAACAGUUUCA | |
| | UCCUGAAACCAUCCCUCCCCUUGCCCAUCAGUGGAAAAAUUGUCUUCCAC | |
| | AAAACUGGCCCCUGGUGCCAAAAAAGUUGGGGAUUGCUAAUCUAGAAAAU | |
| | GCUCCUUGACACCUCAAGUUAGAGUUAGGCGUCCUUUCUAGGUAUUUCCA | |
| | UAGCAACAGUGCUUACUCCUAUCUGAGGACUUUGCACUUCGUAUUGCAAU | |
| | UACUUGUUUAAUUAUUUAUAUCCUCUCACUAGGCAAUGAGCUACUUAAGG | |
| | ACAAGAGCUAUGUCAUGUCCAUCUUUAUAUCCCCAAUGACUAGCACUAUG | |
| | CCUAGCACCUAUUAGGAACUCAAUAGAUAAUUGUUGAAUGAAUAAGUGAA | |
| | UGAGUGAUUGAGGUAUUCAUAGGGUAUUAUAAGAACACAGAGAAGAGACA | |
| | CCUAAAGAUACUUCCUGGGAGAGGUAGCUUCUAGGGGAAGUUAUAAAUGG | |
| | UUAAGUCUGAAUUAUCCAGGAUUAGGCUGGUUGGACAGUAGAGAGAGACA | |
| | UUCCAAGCAGAAGGAUAGCAUGAGAAAAAGAAAGCCACAAAAAACAAAAG | |
| | AGGCAUGCCAGAAAAUGCCCAGCAACACGUGGAGAGGACACACGCUAAGG | |
| | CAGCCGUAAGGUCAUGAGGGAUGGAAGGUUCCAACCAAACCAGACUCAAA | |
| | CUCCAUUCUUCCCUUCGUUCCCAUCCAAAUAAAAGAAUGAACACACAGAU | |
| | CAGAGGAGUCCUAGGAGCUGAUCUCCAGGGUUUACAUACUGGUUACCAAC | |
| | AUAUUACAUAGUCCCUAAGUCACAAACCAUGCCAAAAACUCUUCCUUGAU | |
| | ACAAGAACUAUGUCUUACCCUGCCCUGUGAGGUUCAGUGAAUGGGACCUU | |
| | CCAGGGAAAAGGUGCCAGGUCAUCUUCUUAAUACACUCCUUGCAGAAGGU | |
| | UUCUGUCUCUCAUUACAAAUGGUGGUAAAACACCUUGUUCGCUUUGGUAG | |
| | GCCCACCUUUCCUCUGCCCAGCUUCUUCCUAAAACCUAGCUCCUUUUGUU | |
| | CAACCUGAGCAACCCUUAGGAGAGCAUGUUGCCUUAUUUCUCCUUACUCC | |
| | AUUGUUUUUUGUCUUGUUUUUUGAGAUGGAGUCUCGCUCUGUCACCCAGG | |
| | CUGGAGUUUAGUGGCACAAUCUCAGCUCACUGCAACCUCUGCCUCCCGGG | |
| | UUCAAGUGAUCCUCCUGCCUCAGCCUCCUGAGUAGCUGGGACUAUAGGCA | |
| | UGCGCCACCAUGCCUGGCUAAUUUUUGUAUUUUUUAGUAGAGAUGGGGUU | |
| | UUACCAUAUUGGUUGGCCUCGAACUCCUGACCUCGUGAUCCACCCCCACC | |
| | CCACCCCACCUCCCAAAGUGCUGGGAUCACAGGUGUGAGCCACCGCACCC | |
| | AGCCCAUUGUCUUUUAAAAUAUUAAUUUCAGUGAUUUCGUCAUUUACAUU | |
| | CAAUGCAACCAAUAUCCUAUUUAAGGUAAUUCCAGAGUUUCAGUAAAACA | |
| | UCUCUGAUUACCCACUUCAAGUAAGAAAUUCUAAGAAGAUCAGCUGAUAG | |
| | AGUUCAGUCAUUAAUUCAUUCAACAACUAUUUAACACAUCUGUGUCAAGC | |
| | ACUGUGCUGGAGUCUGGAAAUUCAAAGAUGAAUGAGAAAUGGUCUCUACU | |
| | CUUAAACUGUCUGCAGUGUGGAGGAAGUUCUAGGAUGGGGAAAUUGCUUA | |
| | UGUGGGGCAGUGCUGAGGCUACUGCUGACCUACCUAAGCCAGCACUUCAG | |
| | GUUUAUGCAGGUCCCUGAAUCAGCAAUACAAGCAUCACCUGGAAACUUGU | |
| | UAGAAAUGCACAUUCUUGGGCUCUUCGUACAAACCUACUGAAUCAGAAUA | |
| | AGAGAGUGAGGCCCAGCCACCUAGGUUUUAACAGGACCUCCCAGGAAUGC | |
| | UGAGGCUCACUUUGGUUUGGGAAUCUCUGGUCUAAGCUCUGUUCUGAAUC | |
| | ACACCAGGCCUAUAUGGAAGGUGAUUAGCCAUUCCCAGCCUUCUUCUCUA | |
| | CAAUCCCCCCUUCUUCUUUUAGGAGAUUAACAUGAAACAUCAAUCCAGAG | |
| | UGACUACCCAAAAUGAAGUAAAAAUACUAUUUUCUCACAUAAUAGCAACA | |
| | CAUGACACUGCUCCCAAAUCACAGAGACGGUUUUAUGAAGAUCAGUAUGG | |
| | UAUGGACUAAACCGACUAGAGCCACAUCAGCAUCAACACAGAGGGAUUCA | |
| | GAAAGGAUAAUAUCAUCAUUGUCUCCUGUAUUAGUCUGUUCUCACAUUGC | |
| | UAUGAAGACACACCCGGGACUGAGUAACUUAUGAAGAAAAAAGGUUUAAU | |
| | UGACCCACAGUUCCACAUGGCUGGGAGGCCUCUGGAAACUUACAAUUGUG | |
| | GCAGAAUGCAAAGGAGAAGCAAGGUACCUCUUGCAUGGCAGCAGGAAAAA | |
| | GAGCCGCAGGGGAAACUGCCGCUUGUAAACCAUCAGAUCUCGCGAGCCCU | |
| | CCCUCACUAUCACGAGAACAGCAUGGGGGAAACCGUCCUCAUGAUCCUCC | |
| | CAAUAGCCCCCAAUCACCUCCCAUCAGGACCCUCCCUCCACACGUGGGGA | |
| | UUACAAUUCAAGAUGAGAUUCAGGUGGGGACACAAAGCCAAACCAUAUCA | |
| | UCUUCAUUCUCCCCCCUGGCACUUGAGGGGAAAAAAGAAUCAUAUCAGCU | |
| | UGACUAUUCAAAAAAGAAGCAAGAGCGCCACAGUUAUUUCAGUGCAUGGG | |
| | AAAAGGGAUAAACUGAAACAAUAAACUUGAACGUUUAGGAAUAUAAAGGU | |
| | AAGGGGGAGAGAGAUAACAAUCUUGGCCUCUGAGUUCUGCAACCCUAUGG | |
| | UUGUUUUAAACAAAUGAAAGCAGAUUGCUUUCUUCCUGGAGUUCUAGUGA | |
| | AAUGUAUCUUGCCACCAGCAGACCGUUCGGAGCUCCUGGCCACAGUUUAU | |
| | UGGCUCAUUGCCACGUGUCAUUAGUGACCUAUUUUCGCUGCUGUGAAAUC | |
| | GGCCAUGUCUGAAGGCCCCCGAAUCAGCAGUUUCUCCCUCCACUCACCGG | |
| | CUCCAGGCUGGCAAAGCUGACACCAGUGCCACAGAAAAUGCAACCCGCUG | |
| | CCCUCUCCCCAAAAUGUGGGGAGAGCACGGUUUUGCUAUGAAGAUGUCGG | |
| | CUUUGACCUGAGGCUGACUUGAGAGCCCAGCUGUCAUGUCUCUUCCAAAU | |
| | AAGCUGCUGAACUUGGAAUUAUCUUGGCUGAAUAAUUAAUCAGGACUAAA | |
| | AAUAACCUCAAACUGCCUGGGCUUUGCUUAAAUCCUACACAGGUGAGAGA | |
| | AGUAGGUUUUUUUUUUAACGUAAUUGCAAAACCCAUAUUUUGAAAGGCUC | |
| | UGCACCAAAAUAUGAAAAUAAUUACCCAACAUGCCAAUUUGGGAGAUAAU | |
| | UCACAGUUAAUCUGGCUCUCUUAAAAAAAUGCCACCUUUUGAAUUUCACA | |
| | UUGACUUCAGAUUGUCAGUUCUUGGUGGAUAUGAAAAGCAGCCGAGAGUU | |
| | GUACUCAAAAUUUUCUAAGAAUACCUUCAAACACAAAUCCAUGGGGAAAA | |
| | AUAUAUCCGUUGGUAUAAAGAGCGCCUGUUACCAGGCAGAUUAGACAGUG | |
| | AGCCGAGUUCCACAAGAGGGCGCCUUCCCCAUGCUUCCUUCUCGAAGGUG | |
| | AGGGCGCUGCGUUUCCGCCCCAGCCUUGGCUGCCCUUUUUUUCCUUUGCC | |
| | CUUAUACAAACGAUGUGCUCCCAAGAGCGAAUGCUCAGUGUCCUGUGGAA | |
| | UGCUACUCCUGUGACCACUUUCUAGCUGAGAUCAUCAGGCAAAUGUUAUC | |
| | UCUGUAACAUCAGAGGAGCAACUUCAAAUGAUGAGAAAUCAAGCAAAACA | |
| | UUAAAAAUACAGGGGGAAAAAACAAAUACAUCUAAACUGCCUUCUCUCCA | |
| | UGGUGAUCUAGCUUUGUGUGUUCAUUUUCUUGAUAUUGCUGUGAUACAAA | |
| | UAGGAUGUGAUGAAAUGAAAGUACAUCCAUCACAGCAAUCUUGUGUCCAG | |
| | CCUGAAUCUCAGACCCAGAUGUGGCUCCAACCAACAAGUGACCAUGAGCA | |
| | CGGGGCCCAGGUGUAUUCUGGGAAGCUGCUCACCCUCACUGCCAUUUCUU | |
| | UCAUCCCCGUAGGCACAGCAUCACCUUACAUCGAAGACAGAUUCAACGAA | |
| | UCUGCCUCUCAUCCUAGGUGGCCACUAGGCAACUCCUCUGGGACCAUUUC | |
| | UUCCUGCUCUAUGCUCCCAUGACCUUCAUUCAUACUCCCUCUUCCCCUGC | |
| | UGGAAACCUACUUCUUUUACCUCCUCAAAGGUAAAAGGACCAGCUGGUGA | |
| | ACAAAAGAAUACAUUUGCUCUAUCUAAAGAGAAGCAUUAGAGCAAGGGAG | |
| | GAAAAACUGAUGCAAGACAAAUGACUUGGGCUCAUCAUAAAAUACUGAGC | |
| | AGUCAGCAGAACAUUAAGAAGAGACUUCUACUCCCCACCACACACAUAAC | |
| | CAUCUGCACACCUGAAUGCCGAGACUAAGCCUCACCAGACGCCAGCAGCA | |
| | UAAAUGAGAGGAGUCACUUACCCCUCUGAGGCUCAGGCUUCUCGACUGGA | |
| | AAAAAAAAAAGAUAAUAAAAAUAUCAGUUCAGUUGGGCAUAGUGGCUCAU | |
| | GUCUGUAAUCCCAGCACUUUGGGAGGCCAAGGCGGGUGGAUCACCUGAGG | |
| | UCAGGAGUUCAAGACCAGCAUGGCCAACAUGGUGAAACCUUGUCUCUAUU | |
| | AAAAAUACAAAAAUUAGCUGGGCAUGGUGGUGCAUACCUGUAGUACCAGC | |
| | UACUCAAGAGGCUGAGGCAGGAGAAUCACUUGAGCCCAAGAGGUGGAGGU | |
| | UGCAGUGAGCUGAGAUCAUUCCACUGCACUCCGGCCUGGGCAACAGAGCA | |
| | AGACUCCCUCUCAAAAACAAACAAACAAAAAAAACCUAAUGAAUUGUCAG | |
| | UCUCAAGCCAGUGGGUAGCCAGUGGGUAACCUGGUAAUGUAAUGGUUCUG | |
| | UGAAAGAGAGGGUCCAUAGAAUACAACAAAUGUGUGAUCUUGUUACUUCA | |
| | UUUGCUGUGAUGUGAGCUGAAUAAGAGUCACAAAGAAUAAGAGAAGAGCU | |
| | GUGGUUGUUGUACAGAAAUAAAUACUAUUCAUCUUGAAACUUUCACCUCA | |
| | UCCCCAGAAGUAUCCAAUAGAAUUUAUUAAUGCGUAUUUCCUACCCAGUG | |
| | UAGUCAAUCAAUGUGUAAUAAAAAUGCUGAUGGCCACAGCUUAAAUUAUU | |
| | AAAAUUCUAGCAGGUUUACCAGGUCCUGAGCUUAGAAAUGCAUGCAAAUC | |
| | UCUUCAUUUGAAUAGUUGACUUAAUAUCAUUAGUUCCCCCCUAUAUCCUC | |
| | UGGUUAGAGCUCACCUCUAAUGAAGCCUCAGGAGCUGAUGAUAUUCCCAU | |
| | ACAGUCUUGUUAGUUUUAGAGAGAAAACUCUCCCCACAACCACAAAAGUC | |
| | UUCUCCUAAGCCCUUAACCUAGAGGUGUCACAAAGAGCAUGAGGCAUCAU | |
| | CACUCCUGGGACAAUGACCCUAAAUCCUUGUCCCACUGAGAUAGUAGCCU | |
| | UAUCUUCCUGUCAAUAGGAAACCCUUGGUCAUUUGUUGUGGGCCAAGAGG | |
| | AACCUGUGGCCAGAGGACGUUAGGAGGAAAACCUGGUUUGGCAAAAAUGA | |
| | UGCAGAACAGGGACUUGCCAAAUAGUAAGGGUAAAAAGAAGAUGACUUCU | |
| | AACUGGCUAUGUGGCUGAAAGUCACCGAAAUGGGCAGUUCCACCUUGUGG | |
| | CCUUUGGCUCUCCAGAGGCAGGAAUGAGGAUAAAAAGGUAGUGGAAAUAC | |
| | CUAGAAGAUGAGACCUGAGACCAGCAGAGGCAGAGAGGUCUUUCUGGUUU | |
| | UCUAUGGGAGAGUAGCAUUCUUCAUUUCUCUCCUCUGUGCUCCCUGUGAC | |
| | UUCUGUUUAACUUCUGUCAAAGUAGUUGUGACGCCACAUCACUCUUUCUU | |
| | AUUUGUCUAUGUCCCUCAGUAAACUUCAAGCUCCACGUGGGCCAGAACCA | |
| | GGUUUAAUUUGUUCAUAAUGGUGAAUAAACUUGUAUCCCUUGAACUGAUU | |
| | ACAGCCCAAGCUCAAAAAUGUCUUAAAUGAGUAAAUGAAUGCUGCAUACA | |
| | AAGGUUAGCCCUGAAGGAGAAAUGAGCAACAUAUUCUAAUGGAGAUGUAU | |
| | GUGGCACAGAACUUGGAGAUCAAAGUCAGCCUCUUGGGUAGACUUCAGGG | |
| | AUGGGCUUUAAACAAAGAGGCAGGAAGAUCCAUAGCAUUACUUGGGGGAU | |
| | GAAUAAGACUCUAGAAUUGAGUUGGCCCAGAAAUUUUGGGUACAAGCAAC | |
| | ACUCACUUUGUUUGAUAUGAUUACAGAGUAUUUUCUUCUGUUUACUACUU | |
| | UCUUUAAUUUAAUUAUGUGAUAAAAAGUGAUCACUGUGUGUAUUAGUCUA | |
| | UUUUCAUACUGCUAUAAAGAACUACCUGAGACUGAGUAAUUUAUGAAGAA | |
| | AAAGCUUUAACUGAUUCACAGUUCCACAUGGUUGGGGAGGCCUCAGGAAA | |
| | CUUACAAUUAUGGCAGAAGAUGAAGGGAAAGCAAGGCACAUCUUACAUGG | |
| | CAGAAGGCAAGAGAGAGAGUAGAGGAAAAUACCACUUUUAAACCAUCAGA | |
| | UCUCGUGAGAACUCCUUCACUUUCACGAGUAAAGCAAGGGGGAGACCGCC | |
| | CCCGUGAUCCAAACACCUUCCACCAGGUUCCUCCCCCAACACGUGGGGAU | |
| | UACAAUUCGAGAUGAGAUUUGGGUGGGGACAGAAUCAAACCACAGCACUG | |
| | UGUAUUUCAACUACUUCCAGAGAUGAUGGGCACAUAAGGAGUAAAGAAUA | |
| | GAGGGGGGGACAAUUUCCUCCACGUGUCUGGAAGAACUUUCACAAAAACA | |
| | UACUCAUGGGUCGUUCUACUCCAAGAAGACGGAUAGCUCUUAACACAUAG | |
| | UAAAUAACUUUUCUAUCUGAAGUCUGCUUCUCUUUCUCCAAUGUAAUUGA | |
| | UUUGUAAAGGGGUUUUGCCCCACCCCCCACUCUCCUUGAUUAAGUUCAUC | |
| | CGUGAACACCAGGAGGCUGGUCUGCUCACCCCUACACAGGUGUCCUUUCU | |
| | AUUUUGUUUUUUGAGACGAAGUCUCAGCUCACUGCAACCUCCGCCUCCCG | |
| | AGUUCAAGUGAUUCUCCCACUUCAGCCUCCCCAGUAGCUGGGAUUACAGG | |
| | UGCACACCACCAUGCCUGGCUAAUUUUUUUGUAUUUUUAGUAGAAACAGG | |
| | AUUUCGCCAUGUUGGCCAGGCUGGUCUCGAACUCCUGACCUCAGGUGAUC | |
| | CACCUGCCUUGGCCUCCCAAAGUGCUGGGAUUACAGGCAUGAGCCACCAU | |
| | ACCCGGCCCAGGUAUCCUUUCUUAGAUGGGCAUUACAUCAGUCCCACUAU | |
| | CAGUCCCCAAACUAAGGUCAGUGUCUUAAGACUUGAAAGUCAAUAUGACU | |
| | AUUAGUACUACUUAAGGGGAAUAAUUGUGUGCUACUUUUGGCUUCAGUAA | |
| | GUAUUGUAGGCAUAUCACAGCAAAGGCUUUAGAGGCUUAGAGAGAAAUCG | |
| | AGAUAAUUGAGCAAAGGUGGUGACUUAAAACCAGCUCUACAAUCUGUGUU | |
| | AUUCUCUUCGCUGGAGAGAACCCUGCAAAAAGGGGCAGUUAGGAGUGAAG | |
| | CAGAUAGCCUAUGUUCUUGAACCUGUGAGCACUCACUGGUGACAUGGUUU | |
| | GGAGAGUAGAAAACUGGCCUAUGCGUCUGGACACCCAGCCGCUGUCAACU | |
| | UCAGCAAAGCAAAGUUUGGCUUCCCAGCAAGCUAAGGAGGCCUUUUCGCC | |
| | CUUGCACUUGAUGUUCCCUUAACACGGAAUGCUCUGUCCCAGACCUGUCU | |
| | AUGGCUGGCUCCUCCUUCUUCUCGGGUCUAAUCUCAAAUGCGACUUCCCC | |
| | AGAGAGGCCUUCCCAGAUAGUCCCCUCCCUGGUCAUUCUCUAUCAUGUCG | |
| | UUGUAUUUCACUCAUGGCACUCAUCACCAUCCAAAGCUGUCUUUCCAUGU | |
| | ACUUGUUUAUUUGAUUAUUCACUGUGUCCUUUUGCUCCAUAGAUAGAUAA | |
| | GAUUCAUAAAAGCAGGGCCCCUGACUCUCUUGUUUGCUACUUUAUUACAA | |
| | GCACUUCAAAUAAUGUCUGGCACAUAGUAGGACCUCUAUCACUGUGUACU | |
| | GGAUGUACGAACAAAUGAAUGAAUGAAUGAGUAGCCUGAAUAUGGGGCUC | |
| | AAGAUGGCAGUGAGAAAAGUGCUGUCCCAUCAUGAAUAGGGUAGGUUUUA | |
| | UCUCCUGAAGAGGAUAUGUGCAUUUUCUAAAGGAAAGGAGGUAGGGAACA | |
| | AGGAAGAUAUCUGUAUCAUCGGAAAAGAUCUUUUAUUCCCAUAUUGAAGC | |
| | ACCAACACGUCCUCCCCAACAUGCAUUUCUGCAUGUGGGUGACAUGCCAG | |
| | AAGGCAGGCAGGGAGGGGCAGGCAUUAAAGAACUAACAAACUCACCAAGU | |
| | UAUCACUGUGGUUUUAAAGUGGGGUGAUUAAAAAAAACAAAUGUUGGUGA | |
| | GGAUGUGGAGAAAAGGAAAUCCUAGUGUGCUGUUGGUAGGAAUGUAAACU | |
| | GGUAUAGCCCUUAUGGAAAAUUGGAUGGAGGUUUCUCAAAAAUCUAAAAA | |
| | UAGAACCACCAUAUGAUCAGGAAAUGAAAUUGGUAACUCAAAGAGAUAUC | |
| | UGCACUCCCAUGUUCAUUGUGGCAUUAUCUACAAUAACCAAGAUACCGAA | |
| | UCAACCUAAGUGUCCAAAACAGAUGAAUGGAUAAAGAAAAUGUGGUAUUU | |
| | AUACACAGCGGAAUUUGAUUUAGCUUUUAAAAAGAAGGAAACCCUGUCAU | |
| | UUAUGACAACAUGGACGGACCUGGAAGACAUUAUUUAUUUAAAUGAAACA | |
| | AGCCAAACUCAGAAAGAAAAAUACUGUAUAAUUGCACUUAUAUGUGGAAU | |
| | CUAAAAAUAGUCACACUCAUAGAAGCAGAGACAUAGUGGUUACCACGGGC | |
| | UGGGGGGAGGGAGACAUGCGGAGGUGUUGGUCAAAGGAUAUACAAUUUCA | |
| | GUUAUGAAAGAUGAAUAAGUUCUGGAGAUCUACUCUGCAACAUAGUGCCU | |
| | ACAGCUAACAAUAUGGUAUUGUAUACUUGAAAUUUUCUAAGAGAGUAGAU | |
| | CUUAUAUUGAGUGUUCUUACCACAAUAUAAUAAUAAUAACAAACGGAUGG | |
| | GAGGGAACUUUGAGAGAUGAUUAUUUAUGUCUACAGGUUUGAUGGUUUUA | |
| | UGAGUGGACACUUAUCCCCAGACUCACUGAGUUGUUUACAUUAAAUAUGU | |
| | ACAUGUUUUACAUGUUAAUCAUACCACAAUAAAAUGUAAUAGAUUAGACA | |
| | GAUAGAUAGAUAAGCAGGGUGCUUACAUACAGCUCACAAAAGGCAGAGAA | |
| | UGGCUUUGUCUUGCCGUUUGAGUGCAGUGAGAGAAUAUAAUUAAAGUGAC | |
| | CAAAAAUCCCUGGGAAUUUAUUCAAUUCUUUUGAACAAUGACCAUCCUUG | |
| | GGAAGGGAAGUCCCAUCCAUAUCAGGAAGGGGACACCAGCAAGCUUAAUC | |
| | AUCUUGCAUUUAAAUGUUUUCCAUCUAUACUUAGAGCUCAACACACAAUA | |
| | CAAACACAGCCUUCAACACUGGGACCCUCUCCACCCACAGCACGUGCCCU | |
| | CUGACCUGCUCUCCAAGCAGUGGUGGUGGCCUGCCUCUGUCCAGAAGCAC | |
| | ACCUGCCACGGCCCAGUUCUCUCUCAGUAUGUUUGGUAGAAGGAUUAAGG | |
| | GUGUCCAUCAUGGUUGCUAAACAGUACCAAGCUAGUGUCCUAUUAUAAAA | |
| | UUACUUUCACACAUAUAGGAUGCAAUGGUUCUAAUGAGGGGAAAGGCAUG | |
| | CUAAGAACAAUUACCCCCCCUUAGAUUAGCUUAGUACUUUAUAUGGUUAA | |
| | CAAAUCUCUUUUACCAAAAUCAUCUCAUUUUAAUUUCAGUCCUCAUCACU | |
| | GAAAUUCAUGUAAAAAAAGAGCAGGUGUUUUUCCAUCAUCGUUUUGUAGA | |
| | GGAGGAAUCUAAGGUCAAAACAGGUGAAUGACUUGUUCCUGGUCACAGAA | |
| | CUGGGAGGACCCAGGUCUCCUUGUCUCCAAAUACACUAUUAAAAGUCAAG | |
| | GUAAUGGGGAAAGAUCCAUGGCUGACACCCUCCUUGUUACAACUGAAUUG | |
| | CCCAUAUGUAAAGCUUUCUGCAGAUACAGAAUAACUUAUUUUAUAAUUUU | |
| | AUUGUGUUGUUGGAUCAGGAUCAAAGGCCGGACAAGCUUAUCACUGGAGG | |
| | CAAUUAUUGAAUGAUGAAAAGAAGGAAGGAGGCAAAGAAGAAACGAAGUA | |
| | AGAAAAGUCUUCCUUUUUAAGGGAUCAGGCUGGCUACACAGCACAUCCAU | |
| | AUCCUGUAUAUGAGAAUGAGAGCAGAAUAGGGACCACUACAAUUGACUGU | |
| | CACCCAGUAAGUACUAUGAGCAUAGCAGUUUGGAACUGACAUGGGCCUUG | |
| | GAGAUCAUAAUAAGCUGUGCGGUUUUGCUUGCUAAGUUACUCAUGCCCAC | |
| | UGCCACAUCAGCUGAUCUGAACUCACAUUCCCAUACACAAACAUUGAAAA | |
| | CAAAUACUUUCUGGACUAAAAAUAAAGUACACAUAAAAGUCUGUUUAAGU | |
| | GAAUCAUCCUUCCUUGGGAAGCUAAAAAUGGGUGGGAGGGGAGCAAUCUG | |
| | UAGGCUGCAGAUGGGAUGGGUGGCAGUGUGGACAGAGUCUCUCUUUGAUC | |
| | CUCUGAGAAUUCCCCAGUUGAAAAUGCUGUGGGGAGAUGAAUCCUUCUCU | |
| | CAGAAUACAGGCUGCAUCUGACCUUCCUCCUUUGAGUGUUAGCUUCUGCU | |
| | CACACACUCUGUCUCUCUGUCUCUCUCUCAUUCUCUCUCUGUCUCUCUGU | |
| | CUCUGCCUCUCUCUCUCUCUGCCUGUAAAUAACUUAUUCUGUAAUAGAAU | |
| | UGUGGCUGAGAUAGGGGAGGCUGGAAAGUCCCAGCCUGGGGUUACGCAAG | |
| | GAACUUUAUUAUUUCCAGUAAUGUUAGGAGUUGGCUAAAUGGUCUGUGAA | |
| | AGAGACUUGUCCCUUUUCCUCCUGCCUUCCAUCACAUGUUCAUUCAUUUA | |
| | UUCCCGCAUUAAUUUAUUGUGUAAACAUUCACUGAUAGUUUCUGUAGGUA | |
| | CACAUAUGAAUAAGAAAGGGUCCCUACUGUCCAGAAGCUCUACUCUUAUC | |
| | AUUUCCAGUAAUGUUAGGAGUUGGCUAAUUGGUCUGCAAAAGAGCCUUAU | |
| | UCCUUUUCCUCCUGGCUUCCAUCACAUGUUCAUUCAUUUAUUCCCGCAUU | |
| | AAUUUAUUGUGUAAACAUUCAUUGAUAGGUUCCGUAGGUACACACAUGAA | |
| | UAAAGAGUCCCUACUGUCCAGAAGCUCUACUCCAGUGAAAAGGAGCAGCC | |
| | UUUGGGAGAGAUGCAGGAAAUGAGGCUGAAGGGUGUUUGAGAAAGGGCAG | |
| | AAUGGCCUCAUAUGCCACUCUGAGGAAUUUGUGCUUGAUUCUACUGGUCA | |
| | GGUAAGGAUUUCCCAGACUUUGGUAUGGAAAACUGUAUGGAUCAGUUUUU | |
| | UAAAAGAAUUUAAUUAAAUAUUCUUGGUAUUGACAACUCAUUUUUACUAC | |
| | AUUAUUUAAAUAUAUACAAACAUAAAGCAUGUUAAAAAAAACUUCAGAUG | |
| | AAUUAAAUUUAACAGUGUUUAACUGAGCAAUAAGCAAUUUGUGAAUCAGG | |
| | CAGCCCUCGGAAGCAGAAGAGGUUCAGAGAUCUCGGUCCACAAGGUUGGA | |
| | CAGGCAGCCUUAUGGACAGGAAACAGAAGUGAGAUACAGAAACAGCUUGA | |
| | UUGGUUACAGCUCAGUGGUUGCUUUAUUUGCACAUGGUCUGAUCAGUUGG | |
| | UAGCCUGUGAUUGGCUGAGACUUUACUCCUAGUUAGGCUUUCAGUUUGUG | |
| | UGUGUACUAAGUUAGGUUGUAGUUUGUUACCUAGGCACUCAAGGUAGGGA | |
| | GGCAUUCUCAGACCAAAUUUAGUUUAACCAUGGGGUAUAACUUCCCAGUG | |
| | UGUAUACCUUAUGCAAUUAUAAAUCCAAAAGAAUUAUAAUGUAAAACCAA | |
| | UUAAAUGCAAAUUAACAAAAUUCAUUUAAUGCAACGAAUAUUACUGAAAC | |
| | AAAAUGUCUGCUGCCUAGGAAUGUGGUACUGACUGUAAGCAUGUGGAAUA | |
| | UUUUCAGUUCAGGUUGUCUCCGUGACGUGCUGAGCAAUGACUCAGUAAUG | |
| | UCACUCCUCCCUAGUCAUACUAGCUUGGAACCAACCAUUCAAAGAGGCUG | |
| | UGGUGAUGUCACAUGGCCAGCACUUCCCUUGUCUAUAUGUUUGGUUCUGG | |
| | GGUAAUUCUUUUUUUUUCUGUCACCCAGGCUGGAGUGCAGUGGAGCAAUC | |
| | UCAGCUCACUUCAACCUCCAUCUCCUGGGUUCAAGCGAUUCUUGUGCCUC | |
| | AGCCUCCCGAGUAGCUGGGACUAGAGACACAAGCCACCAUGCUCGGAUAA | |
| | UUUUUAUAUUUUUAAUAGAGACAGGUUUCACCAUGUUGGCCAGGCUGGUC | |
| | UCAAACUCCUGACCUCAGGUGAUCCACCCACCUUGGCCUCCCAAAGUGCU | |
| | GGGAUUACAGGCAUGAGCUACCAUGCCCAGCCUGUCUUCAGGGUAAUUUU | |
| | AAAUGCAAAGGCAGUUGUAAAGACAAAUCAACUGGCAGCAUUUAGUUAUA | |
| | CAAACAGUCAUCCAAUGAUCUAGAAGAUGCAUCUAUUUAAAUUUUAAACU | |
| | GUCCUUGAAAUUAAAGUAUUUUUUUUUUAAUUUCUGAUGAUACCUUCAGU | |
| | CCCAAGAGUACAUCACUGAAGCCCUUAGCAUGCAGUUUGAGGAAAUAUUA | |
| | UGUAAGUGCUAGGGUCCUGCAACAGACAGAUUUUGGGCAAGGAAGUGACA | |
| | UGAUCAGAUUUUCAUAUUAGAGCAAUUACACAAGGGGCGGUAUGAUGGAU | |
| | AGAGUAAAGGAGAGAAAAACUCAGAGUAAGGAGGCCACUUGGGAGACAAC | |
| | UACAAUAAUUUAGGCAAAGGAUGAUGAAUGAUCUAAACAAAGUCAGUGUG | |
| | AGUGAUGAGGCAUAAGAGAAUAGACUAGAAACGUUGGUAAAAGGCAGAAU | |
| | CUGUAGAACUUAGGGACUGAUUAAAUGUGGCAGGUGAAGGAGAAGGGGUA | |
| | UUUCAACCUUAGGCAACACUAUUAACCCUAGAUGGGAAUAUAAGGAAAGC | |
| | AAGUUUAAUUUCAGAUAUGGUGACUUGGGGGUGGGGAAGCUGUGAACUAU | |
| | UGGGGUGAGGCCAGUCAAAUACAGUGGGCUAGAACUGCAGAGAAAAAGCU | |
| | UCUAUGUGAGGCUAGCGUGCUAGCAUGGCUGAGUAGACACCAGCCUACAG | |
| | AGUGUAAUUUCUAGGGCCAAGAAACUCUCAUCACCCCUGCCCCCCCACCC | |
| | CCACCAAAUAAAAUGUUUCUCCUACCUACAGGUCACUACUACUCCUAUCA | |
| | UCUAACUUAAUGCUCUGAUACUUUUUUUCUUUUGGAGACAGAGUCUCUGU | |
| | UGCCCAGGUUGGAGUGCAGGGGCACAAAAUCAUAGCUCACUGCAACCUCA | |
| | AAUUCCUGGACUCAAGUGAUUCUCCUACCUCAGCCUCCCGAGUGGCUGGG | |
| | ACUACAGGUGUACACCACUCUGACCGGCUAAUUUUUUUGUAAAUUUUUGU | |
| | UUUGCAGAGACGUGGUCUCUCUAUAUUGACCAGGCUAGUCAUGAACUCCU | |
| | GGCCUCAAGCAAUCCUCCCACCUUAGCCUCCUGGAGUGCUGAGGUUUCAG | |
| | GUGAGAGCCAUAUCAAAAGAGAGUGACACUUACCCUGGGAUUGAUGUUCU | |
| | GGAUCCAGAAAUGCUCCGGUGGAAACAAUGUGGAAUUUGCACUCAUUCAG | |
| | AUCAUAAUUUAGAUUUCUGCCCUGACACUUACUAGGUAUGAAAUCCUAGC | |
| | AAAUCAUUUAUCCUCUCUAUCUUGCUUGUAAAAUGAGGUUUUAAAACCUU | |
| | UAUCAAAAGCUAGUUAGGAAUGUUAAGUAAUAUAACACAUUACAGUAUCU | |
| | AGCACAGUGCUUGCAACAUCUUGGCCAACUAAUCACGUUCAUUCUCAAAU | |
| | AUAAUGGUUAAGUACAUGGAAGAAACCAGACUUCCUGGGUUCAAAUCAUG | |
| | UCUCUACAACCUUCUACAUGACCUUACGUAAGUAAACUAACCUCUCUGUG | |
| | UCAUAAGGAGAUAAUGAUAUUCUGUAAUGCAUAGAGUUAUUGGAGCAAUU | |
| | UGUGAGUCAAUGCACAUGAAACUCAGAAUCGUUUGUCUUGCCCAUGGUAU | |
| | GUGCUAUCUAAGUGUUAGCUGCUAUUGUUGUCAUAAUUACUCCUUCCUCC | |
| | CUUCCAAUGACUAUGCUUCAGAUUCUUUCCUGGUCAUCGUUUUAAUGGAA | |
| | GGAGAGAAAGAUCAUGGCAUGCUGGGCACCUAAGCCACUGAGUGUGAAAC | |
| | UCCAUACGAGAGGGAAAAGGGGGAGUUUGAUGGCCCUAAAGGCAUUCUUU | |
| | GACUGCUUCUUAAUUUUGUCUAAGACAAAUCAGGGUCUGCACUGCCUCAG | |
| | GGAGGGAAACAUAGAGUGACUGAAGUCCAAAGCCUUGUGAUAAAAUGGAA | |
| | AGACUGUCAUCGGAUGUCUGGCCAAGAAGGCAGCUGUCCAGAUGCAGGGA | |
| | CUGUGAGUUCUUAUGAACUAUGACAACACUCACUUUCAAUGAACUUUAUU | |
| | UUUCACUUAUCAGUGGUUAAAUGCCAAAAUUACAUUGGAUUUUGCACCAA | |
| | ACUGGGAAGGGAGGGGGAACAAAAAAACAGCUCAAACUGACUUGCAUUUC | |
| | CCGAGGGCUGAGUCCUGAGAAGUUACAGCUGGGGAAGCCGGAUCAGGCGC | |
| | AUGGGCCAGUGAGCAAACACAGAUUUUUCUGAUAUAUUAAGCAUUAUUAU | |
| | GUCAAGUACCUGACCCUAAUCAACCAAGGUCAUGAACAAACUAGUCCUUU | |
| | UAACAUUUUUAUGAAAGAAACUGCUGUGUGCUUUUCUCUCUGGACUCUCU | |
| | CACUUGGAACACAGAUGUGACCCGGCUCUUCCAAGACUUCCCUCACGGAG | |
| | GCAUUAGGCCGAUGCCCUGAGCUGAGUUUUUGGUGAUUUGCUGCCUCCAC | |
| | UAGGAUCGGCGAAGAAUAUUUUAAAAUAAAUUUCAAUUUGUUUGAUCCUG | |
| | AGAGGAUGCAAUGACUUUUAGCUCAAAGUGGGACAGAGAGGCCUAUUAGA | |
| | GAUGGACACAUAAUUUUGAGGGGGUAAAAAUUUUAGCUCAUUAAACUUUU | |
| | CCCAAAUUAAAAAGCACAGAGAGGAGUGGAGUGGAAAGAGCUUCAUCUUU | |
| | GUGGUAAGAUAGAUUUUAGAUCCAAUUCCAGGUCUGUCCCCUGCACUGUU | |
| | UGAUUCAGGGCAAGUCACUUAACCCCCUGAGCUCACUGGCGUUAGCUAUA | |
| | AAAUAAUGACAGCACUACCUACCUCCUAGGCUGCCAUAAAACCCAUUACG | |
| | AUCUGACCCUGAUGGUUUCUCUCCAGCCUCAACUGUAGCCUCUCCCUCCU | |
| | GAACAGGCCUCCCUUUAAAUACCACUUCCUCUAGGAAGUCUUCUUGGAUA | |
| | UCACUUCUCCCAGUUAAAGUCUAGGUUGUGUACCCUCCUAUGAGCCCAGA | |
| | GCACCUGUGUGUAUCCCAUCAAUAAACUUAGAAACCAUCAUGUUGAUGUU | |
| | CCAGAUCACCCCUGGACUAUAAACUGUUCAAAACAAGAUCAGGCUCUUCU | |
| | AAUUGCAGGUGUCUCCAGAGCUUAAUGCAAGGCCAGACACACAUGAAGCC | |
| | UUCUAUAUGCUUUUGUAAAAUGAAUGAAGACUGGUAAACAUUGCUAUUCU | |
| | GUUGCUAUUUAUAUGGUCCUCAGAUGAAAGUUACUCCAUACGAAGUCUGA | |
| | CAUACUAAAGCAUUCUCCAGGAUGCUUUUCUGAGUUGCCAUGCCUGAUUC | |
| | UUGAUGUUGUUCUCACCACCAGACUACCCCCUUUAUUCUUUAUUCUAGAC | |
| | UCCACUUGCCCUUGAAGCUGAGACUACUGGCUUUCCUCACUGCCUACCUA | |
| | AGACACCUCAGCCAGGAUUCCAAACAAAGUUCAAUGUCUGCACUCUCCUU | |
| | GCCCUUGCAGAUCUAGCAGGGCCCCCUCAAUUCUGUCUCUGCCAAAUCCC | |
| | AUUCAAUAGGAUACACAAAAUUCACUCUGACUUGCUGUCUUUGAAAGCAU | |
| | GAAGGACGUGACAGUCUCAUUGGUCUUUACUGAAGAAAGAUCUAACCCAA | |
| | GCAUCCUCUUCUCCUACUGCCCAGGUAUGGGUCAGAUUCUUUAAGAAUAU | |
| | AUUGGGAAGGGGGAGAUUUAUUUCACAUUACUUCUCUGAAUUGGAACUUG | |
| | CUCUGAGCACCCUGUUGUUCUCCUUUCUACUUCCGUAGGCCUCUCCUCCA | |
| | UUGAUCACUAUCCAGAACUCCCAGACAAUCCCCUUAGAUGGAUACUUCUC | |
| | CCUUUUCAAGGAUUCAGACAAGAGGUUGAUGUGGUACUGGCUAUACUUCA | |
| | AUCAAGUGCCCAGACCCUUCAUUCUCCAUAGCAUCACCACAGAGGAGCAA | |
| | AAGGCAUGGGUCUCGUCUGAACAACCAGGGUAACUCAAGGAGCCAACACC | |
| | ACAUUUUCUUCUUAUGGAACUUAAAUCUGUACACAUCACCUAAAGGAAAA | |
| | UUAUCCAAAAAGGUAGACUUUCCUCAUGGGGUUUCCAUCAUGGACACAAG | |
| | CUUGUUCCUACACAGUGUCUCACAUCUCCAAAACAAUGUGUUCUUCCCAC | |
| | UCUGCAGAGUGGCAAAAACAAGAUUUGAUACCCAACUCACUACUCAGACU | |
| | UGAUCUUGACCUUGGAGCCAUUCCUUCCCUAGGGGUACAUUUCCUUAAUU | |
| | AUGCAUCAUUCAGACUAAGGAAAUUCAGAGGAGCUUCAUCAGGAAGUCAG | |
| | CAAUGGGGGAACAUCUCUAUUCCUCAGUGCAGUCCAGCCUUAUGGUAGAG | |
| | GAGUGCAGUGGCCCAAAGAAGGAAUUCUGAAAUGAAGCAACCAGGACUUG | |
| | AAGAUGUCUUCUCCCAGCUUUGUGACCUCCUCAUGUUACUUCCUGCACCU | |
| | CAGUCUCCUUUUCUGUGAUAUGGUGAUGAUUAUAGUGAGGCUUUAAAGAG | |
| | UUAACACACAUAUAGAGCACACCUUAGCAAAGAUGUGCUCCAUAAAUUUU | |
| | AUAUAUAGUUUUCCGCCAGUGUAGACAUGGCUAUUAUUUUUCUGUUUAUC | |
| | UAGCAAAUAAUCGUUGAAGCAGAUAUAUACACAUUCACAGACACCUCCUG | |
| | AGGGCAAACACUAAUUUCCAGAGACAGUAAAAUCUGGAAAUAUUUUUCUA | |
| | AAUGACUCAUGAGGAAGUCUCAAAGUGCAGUCAACACAGCAAGCUCACUC | |
| | CUGAUGAACUUUCAACCUACACAUUUUCAUUAGGUGCAAAUGGGAUUUUC | |
| | CUGCUCUCUCUGCAUUUUGGCUGAAGCCCCUAAAGUGAAAACAAUAAGUA | |
| | GCAAGCACCUCUAUCCCCAAUCCUACAGAGACCUGCUGGAUCCCAGGGCU | |
| | CACUAGAAGGUGCCCAAACUGAUCUUUCACCUCCAGAGUAUGUAAAGGAA | |
| | GACUUGAAAGUCCCCAAGAGAGCUCGUUGAAGGGAAUGCCUGAGAAUCGA | |
| | GAUCCCUUGGCUUUUCUCUCCCACUGGAGAACUUGACAUGUGUCCUUGAA | |
| | GACUUGAGGACAUAGUUGAACAGAGACCACAGCCUGCCUCAUGUUGAGAC | |
| | AAUUUCUGAGGUCAGGAACCUGCCAAGAACUGCCAAGAGGGCAGGCGAGG | |
| | ACAGAGGCAGCAGGGGAAGCAGCCUUAUUUUCCUUGGUGGGGUAGAGCAG | |
| | GAAUGCAGGGGUUCCUGCAAGCCUGGUGGACACCAUGGAAUCUAUCCUGA | |
| | AACAUCUUGAAAGGAGCCCACCUUAUAGGGUGGCCUGCCAGAGCAAGGAG | |
| | AUAUUAGCAGUGAGAAGCUGGAGUACAACCGCUAAGACAGGAGUUGGGGU | |
| | GGAAGAAGGAAAGUGGGACGCAUGGGGUCGGCUGAAGAGCAUGCACCAAU | |
| | AGCAGUAAACUAGUACAUCAGAAAAGUCCCAAUGCAUAGGAUCCACAAAU | |
| | GCACAUCAUGGAAAAGUGUAAUGAAGGGCCCAGCAUCUGGGCAAAGCCAG | |
| | CCAAGACAGAGCCACAAAGAGCACCAGAUACUUUAAAAAGGCUGCCCAUC | |
| | UUCUGUUUCCCUUUCUGCCCCACUCCCCUACCAUGGAGAAACCAAAAGCA | |
| | GCAAGGCAAAUCGGGGAGGAGGAGUGAGUCAGGAACAAGUGAAAAAACAG | |
| | ACCAUGAGACCAGACUCUCCUUUCCUAUUUUGGGAACCCCAGCAAGGGUC | |
| | AGGCCCGGGCUGUGAAAAUGAGAGAAGAUGUGAAGAGACAAAGGCUUUGA | |
| | UUUAGACUCUAUUGGGUCUUUUCGUACUUAUAGAUGAGUCAUAAUCCUCA | |
| | AUAAAAGACUCAUUUUUGUUGCUUGAACAUGAUCAGACUACAGACGCAGA | |
| | CAGGGAGGGAAGGUUGAGCACAGUAUAUUUCCAAGGUUGUCAUGGCAGAG | |
| | AAAAUGAAGUAGUUUCCUGUUCAUAGCUAUUGGGCUCAAUAUAUGCACAA | |
| | UACAUUGGUUAAGCAACACACAUGCCAAAGCAAAAUCUCAUCAGCCCCAU | |
| | CCCCCACCGACAGAGGGGUCUGAGCUGCUGAUUCUGUGAGUAUGGUACAA | |
| | ACUAACCUAAUCCUCAGCAGACAGGUCUUUGCCCUUAAGAUUGUUAUGAG | |
| | GAACUCUGAAAGUUUCUUUCUUAUACCCCUUUUCCUCCAGCCUGCUGGGC | |
| | AGAGGAGAAAGUGCCCAUCAAUCAAUCUUUAGGUCCACAGAGAUCUACCC | |
| | UAAGGAUUGAGAUCACUGCUCUUAAUGACAUUACUAGAUUGGGAGGCAAA | |
| | GUUCUCUGUCUGCCCCAGUUUCUUUCUACAGAGAGAGAAGGGACUCUUCC | |
| | AGUCACAGGGACAUUGGAUAUUUAAUGUUGGGUAGAGAAAGAGGGACCCA | |
| | AUUUAGUUGGGAAAGAAAGUAAUUUAAGAUCUUCCCCAAGAAAUAUAAAA | |
| | CUUUCUCUGAGAAGCAAAUUUAGAGAUUCAAAUGACUCCAAAGAGGACUC | |
| | AAGAAUGGAAAGAAAUGAUCCUCCCUUUCUCAAAACACUCAUAAUCAUAA | |
| | UCGUAAUCAUAACCAUAUCAUUCCAGCAAAUUUCAAGAUCUCAGCAAGGU | |
| | GGUUAGGCAUGCCCCUUUUCCUCCCUCUUUCUUAUUUUCUACUCUUUUCU | |
| | CUUCGCCCCAGGACAAAGUCAAGUCAUUUUUCAUACUUUUUUCACCUUGC | |
| | UAUUUAUUCCAUUACUCUUUGAAAGCAAGUCCCUGGGUCAACCACGUCUU | |
| | CCUUUCACAAUGACUCGGGAAAACAAUGACAAAUUCCAUGUGGAAACCAG | |
| | AGAGCCGAGAGGAAAGUGCUGGCCAUUUUCUUCCAGUUCUCCACUCUCCU | |
| | GAAAAAAGCUUAUGGCUUGGUAGAAAUUGUGAAUUCUCUUGAAAUAACCA | |
| | GAAAAAGCACAGCUGGAAUAGCAAUAUCUAAACAAAAAUGCAGCUGAUCU | |
| | GCUAAUAUUAACUAGUCUAAAAGUACCAUGAAGCAUGUGGUUUUCAUUAC | |
| | AGCAACAAAGCCACUAUAGUAUGACAAGCAGCCUGCCUUCCAAGGUUACC | |
| | CUCAUUUACAUGAUAUUGUAUUACACAUGGAGUUGGACUCUUGGGUUUAA | |
| | AGAAAAACAAGAGUUUCACAGCAAGAGGUGAAAUGAAGAUUUAGGCACUC | |
| | AGCCAAAGGUUUGAGACUACCCAGAAAUGAUCUGAGCUCACAGUCAUGUG | |
| | AUUCCUCUCUGCUCAGUACUCCAGCAGCGUGCCAUUUAGUCUCCACUCAG | |
| | GUGGAACUCUCACACAUGGCUCUUAUCACAGGUAAUUGUGUACAAGAUCA | |
| | GGGCGUCUCUUGAUCAAAACCACAUCCCAAAUAGCACCUACCACAGGUCU | |
| | UUACCAUCAACUGAGCUCGCGAUCAGCAAGACGAUGUAGUUGGAAAGGGC | |
| | AGACUUUGGAGUCCCAGAGCUGAGUUGCAAGCCCAGCCUCUGUUCCUUUA | |
| | AUGCUGUUUAAUCUUAGGCAAGUUUUCCACUUCUCCAAGCCUCAGUUUGC | |
| | UAAUCUGUAAAAAGGUUAUAGGGAGGAUUAUUACUGCUAUCAUAUUCACA | |
| | GCUAACAUUUAUGGGCAUGCUUAUAAUAUUCCAGGGACUUCAUGUUGUUA | |
| | CACGCAUUAUUCUAUUUAAUCCCCAAAAUAAUUUGAUGAGGAAACUACUA | |
| | AUAUUUUCUCCAUUUACAGAUAAGGAUACUGAGGGUCAAAGAAGCUAAGC | |
| | AGCUUGCCAAAGCUACCUGAUGAGUAAGUGGCUGAGCUGGGGUUUGACCC | |
| | UGGGUGUGACUCCUGUCAAGGCCUGUGUUCUCACUCUCUAGAUAAAGUCA | |
| | ACACUACAACAUGACUAUAACAUGCAAAUAGCAAAAUGGUUAAAGGCAAG | |
| | AGUUUCUACCACUUCCUUGUUGGCCCCUAUCUGGACUUUGGUUUCCUUGU | |
| | CUGUAAAAUGAGAGUAGUAACAGUUCCUACCUCAUAGGAUGGCUAUAAAA | |
| | AUUAAAUAACUUAAUAUGCAUAUGACCUAUGGAGUAAUACUCAGCACAUA | |
| | GUAGUUACUCUGCAAAUGUUUACCACUUUGUAUGUAUUACUAUAAAACAG | |
| | CUGACACAUAGAUGAUGAGAAAUGUGGGAAUUAAAAUAAGAAACAGAAGA | |
| | GCAUGAGAGGCCACCAAACGACCAACAGCUCUGUGGGCACCUGCAAAACC | |
| | CUCGUGGGAAACUUGCAGAGAUGACCAGAGACACUUUCUGGGGUGCUGAC | |
| | UUCCUUUGAUGCAGAGGAAGGGAUCUCAGACAUUAAGAUCUGACCAUAUU | |
| | UUACACGCCUGUUUUACAUCUGUAGCUGGUAAAACCAGGAAACAACUGGA | |
| | UAAGAAAUGUAAUGCUAAACAGAUGGAUACCUUAGGACCAAAGAGUCAGG | |
| | CUAGGGCAGUCCCCUAGUGGCCUUGUCCAGGCCCAGGCACACCCUGGAAG | |
| | CAUCACUUUUGUCUGAUGAUGUCACUACACAAAGUCACAUGGUGACCCUG | |
| | AGGCAGACAAGCCAGUUGGUGCACAGGAAUGGAGCUGACAUGCCUUGUUC | |
| | UUCAACCCCCUGAAGCAUUUUCAGAUCAUACCUAUUUCAGAAGGCUGGAA | |
| | GUGAACCCAGCAAGAGGCUUCUGUGUUGAUAAGUCUUUGUAUCAAAAUUG | |
| | ACCUGACUGGGAAUUUCUUAGCAAACUUUUCAAACAAGAGAAGGACAUUC | |
| | UCUUAGAUAUGUGAUGUGAGUGAGGAUUAAAUGACCUGAUAGAAAAUGUA | |
| | AACUACUUUUAAAUAAUUUAUGGAGUUGCUGUCAUCAUUAAAACCACAGA | |
| | GAUUUAUCAACUCUCAUUUCCGAGUAAGAUUUGAAGGAAUAGAUUUGUAG | |
| | UCUUGAGAUGGGACAGGGCACCAAGAAUGAAUACUUACGAUUCAAAGCCA | |
| | CAAACAACUUUGUCUCAGAUGGUGUUCCUAGAUUUUGCUUCCAAGUGUAA | |
| | AUUUCUGGCUAUAGCAAAGAUGGUAGCAAGUAAGUGAUCAUUUCACCUCU | |
| | UUCCAAACUCCCUACUUUUUUGCACAUGUCAGAUGCAACCUCAGGUUUAU | |
| | AACUUUAAUUUUAAUAUCCAUCUCAAGCUAACCCAGUUUCCUGGAAAUAA | |
| | AUGCCUACAAAAAGAAAAAAAACUUUUAAAGAAAAAUACAAUGAGGCUGC | |
| | UUGUGGGAAGGGAUCUCUUCCUCAAGAAGCAAUAUGCAGAAAUUACCAAA | |
| | AAAGAUGGAAAAAACAUGAAUUUAAAAUCUGACCAUAUUGAGUCUUGUUU | |
| | CCUUACUCAGUGAUUUUCAAAAUAUAGCCUUAUUUUCACAAUACUAUAAA | |
| | AGUAAUGCUGAUUAGUUACUAAAAUCUGGAUAUGAUAUAGACUUUAAUGU | |
| | ACCAAGGAGAUGACAAAAUCUUAUUUUAAAGGCUGUUGAUGAUUGCUUCU | |
| | CAGCCUUUUGGCUAAGAUCAAAUGUAAAAGCUGUUGAUGAAAUUUUUCCU | |
| | AAGAGGAAAGUGUUUUUUCCUUAGCAAAAUGCUUUGAAGAGCCAUUUGGC | |
| | UGUAAAAAAAUAAAAAAAAUAGGUUCUGAAAUAUUGUUCAGAAGGAAAAC | |
| | CAGGAAACCAGUAGUAACUUUGUUUCUCUGCAUAAUGAUGCAAGAAGAAA | |
| | AAAAUAAGUUUCUAAGGGCAGACUGAGAUGGUGGGGACAUAAGUUGGUGG | |
| | AGGAAAUAGGGAGGUAAAGAGAGAAGGCAGGAGGACCAGGUUCAGGAAAA | |
| | UGGAAUCUGGAGGCCCAGGAGAAGAGAGCUCAAGAGAAAUUCUCACUUAU | |
| | GAAAAGGGCUGGGGAUAUCUGCAUUUAGAAAUCAAUGGUGAUAUCCCAUA | |
| | UUUAGUUUUAAAGAAUUUGCUAUGCUCUGCAGCAAUGCAAUCUCCCUGUU | |
| | UUCAAAAACUCUUUGUAAAAAUUAACUACACACAAAUGCUUUCUAUAAAG | |
| | GCAUUUAGGGAAGAUAAAGGAAAAGAAGCAACUGUGUUUCAGUUUUGAGA | |
| | AAAGAAAACCACCUGAGGAUGAGAAGUGAGAUUAAGAAUUUACCAAAAUC | |
| | AGAGAACCUAGGAGAAAGUUACCACAUCCCACCCUACCCUGCCCCUGAGA | |
| | ACUUCCACAGAAAAUGAAUAGCCUCUGGGAGGCCGAGGCGGGUGGAUCAU | |
| | GAGGUCAAGAGAUCGAGAUCAUCCUGGCCAACAUGGUGAAACCCCAUCUC | |
| | UGCUAAAAAUACAAAAAUUAGCUGGGCAUGGUGGCGCAUGCCUGUAGUCC | |
| | CAGCUACUCUGGAGGCUGAGGCAGGAGAAUAGCUUGAACCCGGGAGGCGA | |
| | AGAUUGCAAGUGAGCUGAGAUCGUGCCACUGCACUCCAGCCUGGGCAACA | |
| | GAGCGAGAGUCCGUCUCAAAAAAAAAAAAAAAGAAAGAAAAGAAAAGAAA | |
| | GAAAGAAAAAAGAAAAAAAUGAAUAGCAAAGUAUCCUGAGUUUCCUUUUA | |
| | CAUAUGGAAGCCAUUUUUAUUUGAAUACUAAUAGUUGGAAUGAUCAAUGA | |
| | GGCUGGCAGAAUUGGGGAACACUGAGAAAGGAGACAUCUGGAUUACAUUA | |
| | AAUAAGUGUAAUCUCCCCUGAUGCAAAAAUCUUUCUUAUCAAAGCAAGGU | |
| | UAGGUAGCAUCACCACCUUUCCUGAAGCUGGUGCCAUUAUCAAAAUAGUC | |
| | UAGUAAUCAUUAAGAAAGCAGCUGGUGAAAUUCAAAUAUAGCUUUUAUGG | |
| | CUAGAAAAAUAGUUUUAUCUCAAAAGUGAUUACUCAGGAUGCAGCUGUGA | |
| | GAGCAUGAUUUCCUUCUUUAUGUACAGAGAAAAAUUUCCUAUGAGCUUUU | |
| | GCAGUGGCGAUGGAAAGAGGAAUGAAUCCACAAGUCCUGAAAGUCUUCAA | |
| | GUUUUUGACAAGAUGUGAUUUUAAGGGUGUGGGGGGCUGAGUUGCUCUUU | |
| | AUACCCACCUCUAUACUCUGGAAGUCAUGCCAUUUGAAGUCAGUGGGUUU | |
| | UAAAGAUUCAGGAAACACUACUCAAAGUAUCUGGCUACAGGGCUCAACUU | |
| | GCUGAGAGAGAGUAUUCCUUUCCUUAAGAUUUCUACACAUGUGAUGCAGA | |
| | AAUUGUUACACAAAGUUCUGACCAUACCUGUGUAUCAUUGUUCAGCCACA | |
| | GUCUCUUAUAGAAAAUUGUAAUGAAUUGCUUUUCAAUAUGCACCUGCAGC | |
| | AAGCUCUGAGACAAUCCUCCAGGAAGCCUUCCUUAAUCCUCCCCAGUAAG | |
| | AAGUAGGGUCCCUCCUGUGCUUGAAAUAAUUAUACAGAUGACUAGAUAAG | |
| | AGGCUUGGUGAAAUAAGCUUCUCAUAUGCACAUCUCGCUGGUGCAAAAAG | |
| | UACUUGCAGUUUUGGACCGUGAAUUGUAAGUCGUUAUAACUAGGCUCAAA | |
| | CAUGUCUUUAACCAAAAAAGGAAUCAUUACAAUCAAUAAAUUUCUGCCAG | |
| | UGAGAAAUACGUUUAUUCCUGUAGCCUAAAAAUCCAUGCUUCAGGAUUUG | |
| | ACAAACUUUUGGAGAGCAUUUUCUGCAUCCUGCUGGUUGUGGAAGCAUUU | |
| | UCCCUGCAAAAAGUUGUUGAGAUGCUUGAAGAAGUGGUAGUUUUGAAGUG | |
| | UCAUCUUCUCUUAUUCUAUGCAACAACAAUGAACCAUUUCUUGAUUGGAU | |
| | UGUGAUGUGCGAGGAAAAGUGGAUUUUAUAUGACAACCGGCGAUGACCAG | |
| | CUCAGUGUUUGGACCGAGAAGAAGCUCCAAAGCUACUUCUCAGCGAGAGG | |
| | UCAGGUGAACAUGGGAGAUGAGGCAAAACUUCGUAGCCCAAUUUGUUCAA | |
| | CUUCUGAAGCGUGGGUUGUGAUGUGCAGUCGGACAUUGUGGUGGAGAAGA | |
| | AUUGGGCCCUUCCUGUUGACCAAAGCCAGCUGCAGGCAUUGCAGUUUUUG | |
| | GUGCAUCUCAUUGGUUUGCUGAGCAUACUUCUCAGGUGUAAUGGUUUCCC | |
| | AGGGAUUCAGAAAGCUGCAGUGGAUCAGACCAGCAGCAGCCCACCAAGCA | |
| | GUGACCAGGACCUUUUUUGGUGCAAGUUUGGCUUUGGGAAGUGUUUUGGA | |
| | GCUUCUUCUCGGUCCAACCACUGAGCUGGUCAUCGCCAGUUGUCAUAUAA | |
| | AAUCCACUUUUCCUCGCACAUCACAAUCCAAUCAAGAAAUGGUUCAUUGU | |
| | UGUUGCAUAGAAUAAGAGAAGAUGACACUUCAAAACGACAAUUUUUAAAA | |
| | AAAUUUUCGGUGAGCUCAUGAGGCACCCACUUAUCAGGUUUUUCACCUUU | |
| | CCAAUUUGCUUCAAAUGCUGAAUGACCAUAGAAUGUUUCAGGGGGUGUGG | |
| | CUUCUUAGUGUCCCCAGUAUUUCAUGGAAGGCCUACUCAGGUUGGUGGUA | |
| | UUGGUGUUUACUUGUCCGGAGUGAGAUCUGUUAAAAAAACAAACAGGCAU | |
| | AUUAAAAUAUUAAGAGUGUAUUUGAGGAGACAGCAAUUCACGCAUAGGGC | |
| | AGCUCCAAGCCAGAAGUGGUCAGGGCCCUGCCAAAGAAGUGAAAGAGAAA | |
| | AGCAUUAUAGGGUGAAUGCGAAAGUGAGGCAAAGAAAAUAUUAAGUUGGU | |
| | UCAGAUCAGAUAUUGCCUUAUUUGGAUUUUCUCAGCAGAAAGUCCCUGGU | |
| | AAUAUAAUGCUCAGUUGGCUUGUGACGGGCUGAGCUGAAGUUUCAAUUUG | |
| | UUUACAUAGGAAUGCAGGGUGAUAGAGCCAUCCCAGCCUAAUGACCUCUC | |
| | AUUAAGUUAGUUUAACAAGUCCCACAAGGUUAUGGUGAGUUUGUCAGAGA | |
| | UGUUUGAACCAGAGCAGCUCCAUCUUGAAUAGGGGCUGUGUAAAAUGAGG | |
| | CUGAGACCUGAUGGGCUGCAUUCCCAAGAGGUUAAGGCAUUCUAAGUCAC | |
| | AGAAUGAGAUAAGAGGUUGGCACAAGAUACAGGCAUAAAAACCUUGUUGA | |
| | UAAAACAGGUUGCAGUAAAGAAGCCAGUUAAAACUCACGAAAACCAAGAA | |
| | GGUGAGGAGAGUGAUCUCUGGUCGAUCUCACUGCUACACUCCCACCAGCG | |
| | CCAUGACAGCUUAUAAAUGCCAUGGCAAUGACAGGAAGUUACCCUAUGUG | |
| | GUCUAAAAGGAGGAGGCAUGAAUACUCCACCCUUUAUUUAGCAUAUAAUC | |
| | AACAAAUAACUGUAAAAAUGGGCAACCAGCCUGGCUGCUCUGCCUGUGGA | |
| | GUAGCCAUUCUUUUAUUCAUUUACUUUUCUAAUAAACUUGCUUUCAUUUU | |
| | ACUUUAUGGACUCACCCUGAAUUCUUUCUUGCACAAGAUCCAAGAACCCU | |
| | CUCUUGGGGUCUGGAUCAGGACCCCUUCCCAGUAACAAGUUCUGAGUGGG | |
| | GCCCUACCCAGCCUUAUCUAAUGCCAACCGUAUCACCCUGAAAAUCCAGC | |
| | AUAACUAAUUUGUAUCAUUUGAAAAACAGCCUGUGAAAAACCAUGCCUCC | |
| | UGUAUGUAACAUCCUGGUUCUUGCGAUAUCUCGGUACCUUCCCCACAGGU | |
| | CCGUAACACAUGCUGGUCAUGUUGCUGCUGAGUAAUGGUGAUCACUCUAU | |
| | GGAUAUGCUGCGUCACCUUUACCUCUAUUGUCACAGGAGCUAGGAGGAAC | |
| | UGAGCACAGCAGGUUGGCCAGGGGCUAUGACAGCGUUCCCAUGGAAACCU | |
| | AAUUAUUCUAAUGUGUAAGAUACAUACUAUCCUAUUACAGCAUUUAGUUA | |
| | UAACAGCCUGAGAGUUGCAUUACUUUCAGAAAUGCAUGUGCCUUGUCGAU | |
| | CAAGCUGUAAGUUCCUUGAGAGCAGAAGCCACGUGUCAGUCAACUUUGUA | |
| | UCUCAAUGAAUUUUGGUUGAUAAGCAGUGGGUAAACUUUUGGUGAAGGGA | |
| | UGAAUGAAACCUGAAGGGGAUUGGUAAAUACGCCACCCCAAAAUAUGCCA | |
| | CUUUGGGAUAGGACUAUUUUGAGAUCCGACAGAUGCAGAAAGAAGUCUUC | |
| | CAAGAGCUUCUCUUUCUUAAAAAAUCUGACUCAAAUCAGAAAUUCCUGGG | |
| | AAAUGAGGACUGCCAUAAAUUGCCUCUCCAAGGGAAUUUUUAAGGCCAUG | |
| | AAGAAGACAGAAAGUCACCACUGAGAUGGACCUGAACACACACACCUUGC | |
| | CAAGAUAGCCCCUGGCUUCCACAAGAGUUCCCCAUGCAUUUACCUUCCCG | |
| | UAGUGGACUGCUCUUGAAAGCCCCAAACCCUUUUUUCUUUCUGUCAAGUC | |
| | UCCACAGUUUAUCUUUCUUUGUUUAAAAUGGUAUAUGAGCUUUUCGGCCU | |
| | AUUAGCUUGUUUGUAUGCCUUUUCUCCUGAUAAUCUGUCUUUUGUCAGUC | |
| | UAAUUUGCAGUGCUCCAGUGAAUCUAAAAGGGUAGAGGAAAAAGGGUUAU | |
| | UUGUUUCCCCUAUAAACUGAAUUAUAAAUUCCAUUACAACAGGGUCUAUG | |
| | UCAUUUUAUCUUCUUAGUACUUAACGUAAAGCAGUGUUUAAUAGGUCAGA | |
| | CGUGCUCAAUUUAGGGUAUCCAAAGGGAGCAGAAUUGUAUAUAUACAUAU | |
| | AUAUUUCAUGUGCAAUAUUUACAAUAUAUUUAUAUUCAUAAUAUAUACAA | |
| | UAUUGCAAAAAUCCUAAUAGAAGCUAUAACAUUAUUCUAAAGAUUGCUGU | |
| | UCAUAGCAGGGAUUAUAUCAGUCUGUGUAGAAAGCUGUUUUUAGCAAAUG | |
| | AUAAGCAAUUCCUUUAUAUGUGUUGAUUUACUUCCUAUGGCAAAAACAAA | |
| | UAAUAUAUAAAUAUUAUAUUAUAUAUAAAUAUAAAUAAAUUCCUUAUUAU | |
| | UUAGCAACUGUGAUGUUUUUGUAAAGAAAAUCUUUCACAUAGAAAAAAAU | |
| | AAAAGCUCUUCCUGUUCUUGGUGGUAAAUAGUAUGGAAACCAGUGCAGUG | |
| | CAAUUCAAUUUCAAAGAAUUAAAUAUCACUAAAACUUGUUUAUUUAUUUU | |
| | UUUAUUUUUGAGACAGGGUCUCACUGUGUCACCCAGGCUGGAGUGCAGUG | |
| | GCACAAUCAUAGCUCACUGCAGCUUCAACCUCCCACGCUCAAGCCACCCU | |
| | CCCAUCUCAGCCUCCCAAGUAGCUGAGUUUACAGGAAUGCGCCACCACAU | |
| | GCAGAUAAUUUUUGUAUUUUUUGCAGAGACAGGGUCUCACCAUAUUGCCC | |
| | AGGCUGGGCUUGAACUCCUGAUCUCAAGUAAUCUGCCCACCUCAGCCUCC | |
| | CAAAGUACUAACUCUUAUUAAAUGUUGUGGUCAGAACAACUCUGUCCUUA | |
| | GUUAUAUGGAAGUGACCUGCAGCAGGUGAUGGAGAAGAAUCAGAAGACUG | |
| | ACUAAGCAACUUUCAGUUACUUCAUAUGGAUUUCACAUUGGUGGAAAAGC | |
| | AGGAACAAAAAGAAGUCUUGAUUCAUGCACAGUUGAUAGCAGGAGACCAU | |
| | UUACUGGCCCUCAUGAGAUCCUCCCUGGAAGAGGAUCAAUAUCAAGGGUG | |
| | AAUGAUUCCUAUUUCUUCACUUCCUGUUUGUUCAACCAGAAAUUUCUACG | |
| | UAUGUUGUUGUAUUCCUCAGGACACUUCCAAUUUCAAGUGACAAAAACCC | |
| | AACUCAAACUAAUUUGACACAAAAGGGAAUGUACUGGCUCUUACAACUGA | |
| | AAAGUUGAAAAGUCGGUUCUAGUUCAGGUAUGGCUGGAUCCAGGACAUCA | |
| | GAUGAUGUCCUUAAGAUGCACUUGCUCUACCUUCAUCUUCAGGCUCUGCU | |
| | UCCCUCUCCCUAAACAAAGUUUCUACACUGGGCAGUGCAAAGUAUCUCUG | |
| | CAUCUUCAUGUUUAAAUGGUUCUUUGAUCCCACAAUCUUCAGAAUGACUC | |
| | UCUCAGUGCAGCCCCACAUCAAGGACUCUGAAAUGACAAUGAAUGCCUCU | |
| | GCUUGGGUUAGAUGCCCACUACAGGACACAUCACUAUAUUGGGGAGGAUG | |
| | AGGCAUGAUUGUCCAAUCUGGCUCAAAUGCUCAGUCGUCUGGGAAAAUGG | |
| | CCAUGUGUGUGACCGCUCCACAGACAUGAGAGGCUGGGAUUUCCAACAGA | |
| | AAGAUGCUAGGAAGACAGAAGAGUUGCUUAUGCCCUAAGAUCAAGAUCUG | |
| | UGUAGAGUGUCCCCUCAACCACCCAAAUCACAACCUCCCCAUAUAAGGCC | |
| | UAAUCGGAUGUUUUUUGUUGUUGCUUUUGUUUUUGUUUUUUGUUGUUUUG | |
| | UUUUGUUUUGUUUUGUUUUAGAUGGAGUCUCACUCUGUUGCCAUGCUGGA | |
| | GUGCAGUGGUGCGAUCUUGGCUCACUGCAACCCCCACCUCCCAGGUUCAA | |
| | GCAAUUAUCCUGCCUCAGCCUCCCGAGUAGGUGGUAUUACAGCCACGUGC | |
| | CACCAUGCCCAGCUAAUUUUUGUGUUUUUAGUGGAGACGGGAUUUCAUCA | |
| | UGUUGGCCAGGCUGGUCUCAAGCUCUUGACCUCGUGAUCCACCCGCUUCG | |
| | GCCUCCCAAAGUGCUGGGAUUACAGGCGUGAGCCACCUCGCCCAGCCCUA | |
| | AUCAUAUGUUUUUAAGCAAAAGGCCUAAAAAGCUUCCUCCUAGGUUCUGU | |
| | UCAAAACUGAAACAAAACAAAGCAAAAAAUAAAAAUAAUAAUAAUAAUGA | |
| | UUGAGGAAGCCAUGAGAUUCCAGGCACUAUAUUAAGUUUAAAGGCUUCCA | |
| | AAUGUUCAACUUCUAAUGAAUGAUUCACUUCACCUCAAUGCCUCUGUUUU | |
| | AGCAAAAAGCACCUGUUCUUUAAAACAGAUUUACCUCUUCAGCUGAGAUA | |
| | GAAAUUCUAAGAUAGAAUAAUUUAAGAGCUAGAAAGGGCCAAAUAACAGU | |
| | UUUCAUCAGGGAAAUAAAACAAUGUCCAUUCUCUGGAUUGGAUACAACCC | |
| | AAGAAAAGCCAUUACCCAGCAGUGACAUAUGAAAUAUCUGCUUUGAUUUC | |
| | AUGUGGGAGAUUAUUUAGCAAUGUCAAGACAAAACAUGCCAAUUCCUGGC | |
| | AGUUGUCAAAAUGCCAUUCCUAGUGUAACCCUGACAAAAAAAAAUCCACU | |
| | CCCUCUGCAAAUGUCACCAAGCUUCUGAUGGAAAAUGGAUAUAUUUGCCA | |
| | GUUUGGGAGAAUAAAGAAAAGACUGGAAGGAAGAAAAAGUUUCCCUUUAG | |
| | GAGGAAACUAGAAGGAAAAGGAGGGAGGAAGGAAGAAAGGGAAGGAGAAA | |
| | AAAAAGGGAGAGAAGCAGGGAAGGAGGAAGGGUGACCUUUUAGUCUACCC | |
| | UUCAGAUUUAUGCUAAAAGUUUGCCAGGUCAGAAAACAGGCUUUGGAAUU | |
| | AAGGGAGAUAUGAGACCAACGUCAGGCCCUGCCACUUAAGAGUUACCUAC | |
| | ACUUGGUAACUUCACCUUCAUUUGUCUCAGUGUCUUCACUGGAAAAAUGA | |
| | AAAAUAAUAGUACAAUUAAUAAUGGUUAAAAGAAGCAACCUGGCCAGGCA | |
| | CGGUGGCUCACGCCUGUAAUCCCAGCACUUUGGAAGUCUGAGGCGGGUGG | |
| | AUCACCUGAGGUCAGGAGUUCAAGACAGCCUGGCCAACGUGGCGAAACCC | |
| | UGUCUCUAUUAAAAAUACAAAAAUUAGCUGGGCAUGGUGGCGUGCACCUG | |
| | UAAUGCCAGCUGUUAGGGAGGUGGAGGCAGGAGAAUCACUUGAACCCAGG | |
| | AGGCAGAGAUUGCAGUGAGCCAACAUUGCUCCAUUGCACUCCAGCCUGGA | |
| | CGACAAGAGCAAAACUCCAUUUCAAAAACAAAAAUGAAGUAACCCAUGUG | |
| | CAGUGCUGAGCCUGGUGCCAAUCUGUAGAGAGCACAAUACAUAAGAUCUG | |
| | GUAUUAUAGGCCUACGGAAACAAUGUAUCCUGGUGCUCCAUGGAAAACAU | |
| | UUUCUUUAUUUAAAUUCAUCGACUUAAAAACACCUAGACAGAAAUUAUGA | |
| | AUAGGCUAUGCCUCUUUAUUUCAUAGGCAGAAUGGCAAGUUUUGAAUAAA | |
| | AGUAUGCUCUUGUUUGACACAUGGAAAAUAGGAAAAGGCAUCUGAAGAAU | |
| | CUUCACAUUAUCAAGCCUGAACCUUCGCAGAACACUAGAGGUCCCUCUUG | |
| | GCACUGAAUCCUCCAGCCCCGAUGAUGCAGAAUUCAGAGUGACAAUUUUC | |
| | UGCCUCAGCCCCACUAGAUGUCUCUUCAAUAAAGUUCUAAUACAGUUAGU | |
| | GCCCUGGCAUUGAUUCGAUGCCUGGGAACCAUUACAUAGAAAGAACAUAG | |
| | UUUUAAAUAAUCAAACCAUUUUCCUAAAUGUGCAUACAUUCUAGAAGCCA | |
| | CCCUCCACUGCCACACACAUAAUUCAAACUGAAGAUGUCUUUUGUUUUCC | |
| | AGAACCAUUGCCUGAUAUUAAGAUGAUAUCAGGGAAAGGGAGCAAAGGUG | |
| | ACAAGGAAGUUGUUUCGAUUAAUUGGGAACCAUAGAUCGUACUUGGAAGC | |
| | CAUGGGGGAGAUGCCCUCCACGAAAUUAAAAAUGAAGUUACUCUCUCAGA | |
| | GUUAAAUCACCAAAUCAAUGGGAGUGCUAGCAAUGUGGCUUUAAUGGCCC | |
| | AGGUUCCAGUAAGCAGAAAAGUCAGACUUCUGUCUUCUACAUAGCCAACU | |
| | UCCUCAAAACACGAAAGAAGAGAAUGCCUUUCUGAAGGAAGGCAAUUUCU | |
| | UCUAGUUGCAUCUAAUCAAAACUUUAUUGUGUUUCUAUUGCCUGCUGUUU | |
| | UCCUAAGAAACGUUGAAUUACUGGGAUGAUACCUGAUUCUCACCAGCUAC | |
| | CAGAACCACCUCCUUAGUUUUCAGGGAGUGUUUUUUGACACAAACCACUU | |
| | CAACAGCAUUGCAGCAGCUAGUUUAAAAGGCAAUAUUUUGUCUUGUGUUU | |
| | AAUAAGCUUAAAAACUCACAAGCCCACCUAGCAUUUCUAACCAGCAUUUA | |
| | UCCAUUUGUCUGAAUGGAUCGAAGAUUGCCUCAUGGGCAUAAUAACUGAC | |
| | UUUAGGCAUAAUGGUUGUGAUGCCUGGCACAGUGGGGUAUUCUGUUAGGG | |
| | GAGCGAGAGAGCUGUGGACAGGGAAAUCACGUAGCUUGGGAAGCCUCAAA | |
| | CUACAAAGAUAUAGUAGCAGGUAUGGGCAAGGCACAGAGAAGCUGAAAGU | |
| | GGGGUGCAUGCGAGACCACAACAUAGGCUGGUAUUGUGUUUAAGAAAUUU | |
| | CAAUGUAAAUGUCUUUAGGCAGGGGAUUCACCCGAGUUAGCCACCAUUUC | |
| | CACCAUAUUCCAUUGCUUCCUAUUGUCUUAGGCCUGGGGUAAUUUGAUAA | |
| | CCUCGAACAUCUGGCCCCUUUGGCCUGAGUCAGAGCUGUUUUUUCAGGGA | |
| | GGGUUUGCUAGAAAGUAAAUGAUUGGGACAAUUAGAGAGAUUAGAAUAAG | |
| | AUCUAUAGAUUUAGAUAACACUAUCAUAACAAGUCUAAUUUACUAAUUUU | |
| | GAUCAUUGUACUGUGAUUAUGUAAGAAAAUAGUUAUUUUUAGGAAAUUAA | |
| | CUCAAGUAUAUGAGAGCAAAGGCAUAAAGAUAAAAAUAACUACAGAUGAC | |
| | AAUGUCUCAUUCCAGAUCUGCCAUAUAAGGAUUUCUAGAAGUGAGGCCCU | |
| | GAAACCUGCAUUUACCAUGAUCCCCAGGGAAUUCCCGUGCACCUUCAAGA | |
| | CUGAGAGGCCCGGAUCUGUAACACCUGCUGAAGAAUGACACCCAUUUACG | |
| | AGAUAAAGAAAACACUGUUCUUUGCUGCUCCCCUGGUUACCCUCAAAAGC | |
| | CAGAAAGAAGGGAGGAUGAAGAGAAAGUCAGCCUCUGCAGACAUAGCUGU | |
| | CAUUUGUAACUGCUGCUGUUUCCCAGUCAGCUUAAAUGGAAAAUGGACUG | |
| | CUCUUACACCAACGUCACUUAAGUCCAUCACGGCAGUACUCCAGGUCCUC | |
| | CUUUCCCAUGUAUUGCCUGAGUGGGGCACAUUGCCCCCUGACAGACACAG | |
| | GAUGCUGCCCCCAGGCCAACACUUAGAGCUAGAACAGCGACGCUCAUCAC | |
| | AGAGCUAGAAUUGCAAGCUCAGUCAGGUACAGGGAGUGUUGUGCCUGAUU | |
| | GCAAAAAACUGACUCAGCCUUUCAGUAAAAGUGCCUUAGGGAAUGGAACU | |
| | AGAUUGUCCUGUGACAAACCAAUGAACACAUCUGCUUGUCUCAAAAUAUG | |
| | UGAAGCAGAACCAAGCUUCCAGUGCUGAACACAGGACUGCUCUAGUCACC | |
| | AGGCAGCAUCCUUUCCCAAAGAAGGUUUCAUAGAACACUAGCCUAGUAGA | |
| | UGAUCUUGGCAAAAAGGAACCCAUAACUAAGUAAGUUCUGGAAACGCUGU | |
| | AUUUUGUUUCUCCCCUCUUGGAAACUGAAGAUGCACAUUAACAUAUUAAG | |
| | UGCUCUGAAAAGUCACGCACUGAAGAAAAAACCAGUAUGACUCUUGUUUA | |
| | GCUCAGUAUUUCCUGAAUAUGUUCUUUGAUGAGACUCCUUUUUCAUUGAG | |
| | UGCCUAUUAAUAUUUUCUAGAAUAAGGUUUUAAAGAAAAAAACUGAGGAA | |
| | ACGUUAUAGAGGAGAAUUUAUAUUCAAAGGGGAAGUCAUUUAGUUCCAUA | |
| | AAUGGAACGUUAAUUGGAGAAAGAUUGGCAGUUGUGAAGCACAUCUAAAA | |
| | GUAUUGGCAGAGCCUCUGAAGGACUUGGGAAAUGGACUUCCAUUCCCUGC | |
| | CAGGAAACUUCCUUAGGAAUAAAGGGACUCCUUUGGUCCUGAUCAGAAAG | |
| | GGAGCAGUAUUCCUUGUCUGCAUUAGAGUGGAAUAUUGGGAUGAUUAGCU | |
| | ACCAGAAGAGUCCUCUUUGAUCUUUCCCACUCCUUCAACCCCGUAUCCAU | |
| | UCCACCGGAAAUCCUACCCUACGUCCUCAAUCUCAAUCUCCCACUUCCAG | |
| | UGCUGCAACCUCAGCUCACUGCAGCCUUGACUUUCUGGGCUCAAGCGAUC | |
| | CUCCCACCCCAGCCUCCCAACCAUCUGGGACCACAGGGAGGUGCAAUCAC | |
| | GCCUGGCUAAUUUUUGUAUUUGUAGUAGACACAGGGUUUCUCCAUGUUGC | |
| | CCAGGCUGGUCUCUAACUCCUGGGCUCAAGCAACCCACCUGCCUUCGCCU | |
| | CCCAAAGUGCUGGGAUUAUAGGCAUGAGCCACUGUACCCAGCCUCAAUCU | |
| | CCCACUUCUUACACAUCCACUGCUGCAAUAGUCUACUCACUGGUUUCCCU | |
| | ACUUUCACUUUUAUUCUCCUGCUAUGGUCUGAAUAUUUGUGUCCCACAAA | |
| | AAUUCAUAUGUUGAAAUUCUAACCCUUAAGGUGAUAGUAUUAGGAUAGUA | |
| | UUGGGGCCUUUGGGAGGUGAUGAGGUAGUGAGAGUGGCACCCUCAUGAUU | |
| | GGGAUUAGUGCCCUUAUAAAAGAGGCCCCAAAGAGAGCCCUGAGGUUGCA | |
| | GCGAGAAGGCCACAUCUAUGAACCAUAAAGCCAGCCCUCACCAGACACCG | |
| | AAUUUGCCAGUGCCUUGAUCUUGGACUUCACAGCCUCCAGAACUGUAAGA | |
| | AAUAAAUGUUGUCUAUAAGCCAGCCAGUCUGUGGUAUUUUGUUAUAACUG | |
| | CCUAAACAGACUGACACAACAUCUAGUGUAUUGUCCAUACUGCAGCAAGG | |
| | UGAUCUUAGCACUUAGAAUCUGAUAAAAAUCACACUGCUUAAAAACUUCC | |
| | AAAACCUGUCCCUUGCCAUGGAGAAAACACAAAGAUAACAUAGGCCUGGC | |
| | CUGGCAUUUCCCUUCAAACUCAUCUUCACUAAUCUUCCACUCAUCUGCAU | |
| | GUUCCAGCCACACUCCUCUUCUCCUGGAUUCUCUUUACCUGGAAUUCUCC | |
| | UUCCCAUCUCGAUUUUGCCUAUUUGCCCUUCAAUAUAGAUCACGGUGAUC | |
| | UAUGAUGGUCAUAGUGAUCACAGAUUCAUCACAGAACACUAUGCAAAAAU | |
| | CAUUUCCUCCAGGAAGACUUUCUUGAUUCAACAGUCUAGGUUGGAGCAGC | |
| | UCAAGUGCAAUCCAUACUUCGGUGCCAAUCUACAAAUUGUUUCCAAUGUG | |
| | AUAAAUAUUAAAAUUAAGAAAUAAGCAUUUAGAAAGUUCUACAGUGUUUU | |
| | AAAAUUACCAUGACAUCCAAGCACAUGGUCAGUAGACUCCCCUUGUUGAA | |
| | CAGGGCAAAGAGCAAUUCAGGUGUUGUCAAAAUCACGUGGUCAGUCACAU | |
| | GUAGCACAAUUUGUAUGCUAGUAUCAGUCCAGGGCAGAUUGAAGGGAAAA | |
| | GUGGUCUUUCAGCACACAUAGUUAGAGAUGCACUGGUCUAGGUCACUUUG | |
| | UCAUGUUAUAUAGUCACAGCACCAUGUACCUUCUUUCCAAAGCAUCUAUC | |
| | ACAGUUGGUAAUUUUAUGUAUUUUUUGCGAACUUUUUUCUUAUUGUCUUU | |
| | CUCCUACACAAGACUCCUAGCUUCCUGACAGCAAGAACCAACCAGGCUUG | |
| | UUGCUGCUCAAUGUCAAAUCUUUCUAGUCCAGCUGAAUACCUGGGACACA | |
| | GUUGGUGCUUAAACAAUGUGACUAAUAAAUAAGUGGAAAACGUAUACCUA | |
| | GUGAGCCUUGUCAUCAAUUUUAAUACUGUAUCACCUCAACACCAUUCUUA | |
| | GCACUUACUGAGUUACCUGUGGCUUAUAUGUGAGGAUAAGGGUGAGCAAU | |
| | GAGAGAGAGAUGAGAGAAUAAUUAUUUCAUUUUAUGAACUCAACCAGACC | |
| | CUUUCAGAUCACAUUUCAAUGGCAUAAUAGGUAUAAAAUGAGUAGUGAAC | |
| | AAUGAUGUUUUAAGGGUACAAAGGAAUGCUUUGUCUUUGGUAACCCAUAU | |
| | CUGUGACCUAUUGUCACACAUCUUGGGUAUUUCGUUUUGUUUGAUGUUUA | |
| | UAUUUGACUCUAAUUAAGCAACAUCAUAAAAUUUCAUUGCCCUCCCAGCA | |
| | ACAGAGCUCAUGCCAGCUUUCGGUGAUGUUGUGUUUCCUCACCCAGCUCU | |
| | AUCCUCCCUGCCUUUGUUCACAGCUGUUUUGCUGAUAAAAUGGAGAGAAA | |
| | GAGAGCUGGUAUUCAGCUGACAGCUUGCUUCUGGCAGCUGCUGGACCUCA | |
| | GAGCAUAAUUUCCUCCCUAGUGGGUUAUCAGAAUCAAACACCCUCGACUA | |
| | AUCAGAAUUUCAAAGAAAGCAGAUAUCCAGUUGAAUUUAAGAAGAUUAUU | |
| | CCAAAAGAGCUCUGAGUAGGACAUUGGACUUUUGGCACCCUAACUAUUUG | |
| | CUCAAACAGGCAUCAGAGUCAGCUGGAGUCUUGAACUUUUUCCAGGAAAG | |
| | AGAAGAAAUUUUAAUAUAGUGAAAAGAGCAAUGGAUGGUGGUAGCAAGUC | |
| | UUAGCCCUGCUCUGGGAAAGGUACCUAAUCUCCGUGGUUUGAUUUCCUCA | |
| | UACGUUGAAAAGGGGGAUUGGACCAGAUGACCUCUUGAUCUCUUCUAGCU | |
| | CUGAAAUUAUGGAAAUGAAUGAUUCAGUUCCUAGACAGAGGUUUCAAGAA | |
| | UGCUAAGGAGCUGAGUAUCUGUUCCCAGAUGGCAAAGUUGGCAAAGUUUA | |
| | UAUAGGACUUAAAGGUCUUUCUAUUUACUACUCAUUCAGUUUAAUCCUUC | |
| | UUCUGAAAGACAGUACAGUAUUUACCUCUUGCCUAAUAUUGUGAACAUGA | |
| | AUUUGUUUAUACAAUCUCAUGACUCUGAGAUCUAACUCUAAUUCCAUAAA | |
| | UAGAACUAUAAAUACUAAUGGUAUUCCCAAAAACAUCAUGCUUGGUGAAA | |
| | AUGUUCUAGGAUCAAAUACGUUUCUUUCUUUCUUUCUUUUUUUUUUUUUU | |
| | UUUUUUUUUGAGAUGGAGUUUCGCUCUUGUUGCCGAGGCUGGAGUGUAAU | |
| | UGUGUGAACUCAGCUCACUGCAACCUCUGCCUCCCGGGUUCAAGCAAUUC | |
| | UCCUGCCUCAGCCCACUGAGUAGCUGGGAUUACAGGCAUGUGCUGCCACA | |
| | CCCGGCUAAUUUUGUAUUUUUAGUAGAGACGGGGUUUCUCCAUGUUAGUC | |
| | AGGCUGGUCUUGAACUCCCAACCUCAGGUAAUCCGCCCACCUCGGCCUCC | |
| | CAAAGCACUGGGAUUACAGGAGUGAGCCACUGCGCCCAGCUGGAUCAAAU | |
| | ACGUUUCUGAAAUGCUGUACUCAAUAUCCUCUCCUUAAUGUUUCACAUGG | |
| | CAGAAGGGUGCAUUAAAGCCUAGAAAGUCAUGCGCUAAAGAAAGCCCACU | |
| | UAAGUUUGCUUGCCCUAGAAUUUCUCAGCGUUGAAGUCUUUUUUUUCCUC | |
| | UGGAACACUUGUUUUCAUUUUUAUUUUUUAAUUUUUAUUUAUUUAUUUAU | |
| | UUAUUUAUUUAUUAUUAUACUUUAAGUUCUAGGGUACAUGUGCACAACAU | |
| | GCAGGUUUGUUACAUAGCUAUACAUGUGCCAUGUUGGUUUGCUGCACCCA | |
| | UCAACUCAUCAUUUACAUUAGGUAUUUCUCCUUAUGCUAUCCCUCUCCCA | |
| | GCCCCCCACCCCUCAACAGGCCCUGAUGUGUGAUGUUCCCCUUCCUGUGU | |
| | CCAAGUGUUCUCAUUGUUCAAUUCCCACCUAUGAGUGAGAACAUGCGGUG | |
| | UUUGGUUUUUUGUCCUUGCGAUAGUUUGCUGAGAAUGAUGGUUUCCAGCU | |
| | UCAUCCAUGUCCCUGCAAAGGACACAAACUCAUCCUUUUUUAUGGCUGCA | |
| | UAGUAUUCCAUGGUAUAUAUGUGCCACAUUUUCUUAAUCCAGUCUAUCAU | |
| | UGAUGGACAUUUAGGUUGGUUCCAAGUCUUUGCUAUUGUGAAUAGUGCCG | |
| | CAAUAAACAUACGUGUGCAUGUGUCUUUAUAGUAGAAUGAUUUAUAAUCC | |
| | UUCGGGUAUAUACCCAGUAAUGGGAUUGCUGAGUCAAAUGGUAUUUUCUA | |
| | GUUCUAGAUCCUUGAGGAGUCGCCACACUGUCUUCCACAAUGGUUGAACU | |
| | AAUUUAUGCUCCCACCAACAGUAUAAAAGUGUUCCUAUUCUUCCACAUCC | |
| | UCUCCUGCAUCUGUUGUUUCCUGACUUUUUAAUGAUCGCCAUUCUAACUG | |
| | GUUUGAGAUGGUAUCUCAUUGUGGUUUUGAUUUGCAUUUCUCUGAUGGUC | |
| | AGUGAUGAUGAGCAUUUUUUCAUGUGUCUGUUGGCUGCAUAAAUGUCUUC | |
| | UUUUGAGAAGUGUCUGUUCAUAUCCUUCGCCCACUUGUUGAUGGGGUUGU | |
| | UUCUUUUCUUGAAAAUUUGUAGAAGUUCUUUGUAGAUUCUGGAUAUUAGC | |
| | CCUUUGUCAGAUGGGUAGAUAGCAAAAAUGUUCUCCCAUUCUGUAGGUUG | |
| | CCUGUUCACUCUGAUGGUAACUUCUUUUGCUGUGCAGAAGCUCUUUAGUU | |
| | UAAUUAGAUCCCAUUUGUCAAUUUUGGCUUUUGUUGCCAUUGCUUUUGGU | |
| | GUUUUAGUCAUGAAGCCCUUGCCCAUGCCUAUGUCCUGAAUGGUAUUGCC | |
| | UAGGUUUUCUUCUAGGUUUUAGGUUUAACAUUUAAGUCUUUAAUCCAUCU | |
| | UGAAUUAAUUUUUGUAUAAGAUGUAAGGAAGGGAUCCAGUUUCAGCUUUC | |
| | UACUUAUGACUAGCCAGUUUUCCCAGCACCAUUUAUUAAAUAGGGAAUCC | |
| | UUUCCCCAUUUCUUGUUUUUGUCAGGUUUGUCAAAGAUCAGAUGGUUGUA | |
| | GUUGUGUGGCGUUAUUUCUGAGGCCUCUGUUCUGUUCCAUUGGUCUAUAU | |
| | AUCUGUUUUGGUACCACUGCCAUGCUCUCUUGGUUACUGCAGACUUGUAA | |
| | UAUAGUUUGAAGUCAGGUAGCGUGAUGCCUCCAGCUUUGUUCUUUUUGCU | |
| | UAGGAUUGUCUUGGUAAUGCGGGCUCUUUUUUGGUUCCAUAUGAACUUUA | |
| | GUUUUUCCAAUUCUGUAGAGAAAGUCAUUGGUAGCUUGAUGGGGAUGACA | |
| | UUGAAUCUAUAAAUUACCUUGGGCAGUAUGGCCAUUUUCACGAUAUUGAU | |
| | UCUUCCUAUCCUUGAGCAUGGAAUGUUCUUCCAUUUGUUUGCAUCCUCUU | |
| | UUAUUUCAUUGAGCAGUGGUUUGUAGUUCUCCUUGAAGAGGUUCUUCACA | |
| | UCCCUUGUAAGUGGGAUUCCUAGGUAUUUUAUUCUCUUUGAAGCAAUUGU | |
| | GAAUGGGAGUUCACUCAUGAUUUGGCUCUCUGUUUGUCUGUUAUUGGUGU | |
| | AUAGGAAUGCUUGUGAUUUUUGCACAUUGAUUUUGUAACCUGAGACUUUG | |
| | CUGAAGUUGCUUAUCAGGUUAAGGAGAUUUGGGGCUGAGACAAUGGGGUU | |
| | UUCUAAAUAUACAAUCACGUCAUCUGCAAACACGGACAAUUUGACUUCCU | |
| | CUUUUCCUAAUUGAAUACUCUUUAUUUCUUUCUCUUGCCUGAUUGCCCUG | |
| | GCCAGAACUUCCAACACUAUGUUGAAUAGGAGUGGUGAGAGAGGGUGUUC | |
| | UUGUCUGGUGCCGGUUUUCAAAGGGAAUGCUUCCAGUUUUUGCCCAUUCA | |
| | GUAUGAUAUUGGCUGUGGGUUUGUCAUAACUUGCUCUUAUUAUUUUGAGA | |
| | UAUGUUCCAUCAAUACCUAGUUUAUUGAGAGUUUUUAGCAUGAAGGGCUG | |
| | UUGAAUUUUGUCAAAGGCCUUUUCUGCAUCUAUUGAGAUAAUCAUGUGGU | |
| | UUUUGUCUUUCGUUCUGUUUAUAUGCUGGAUUACAUUUAUUGAUUUGUGU | |
| | AUGUUGAACCAGCCUUGCAUCCCAGGGACGAAGCCCACUUGAUCAUGAUG | |
| | GAUAAGCUUUUUGAUGUGCUGCUGGAUUUGGUUUGCCAGUAUUCUAUUGA | |
| | GGAUUUUCACAUUGAUGUUCAUCAGGGAUAUUGAUCUAAAAUUCUCUUUU | |
| | UUGUUGUGUGUCUGCCAGGCUUUGGUAUCAAGAUGUUGCUGGCCUCAUAC | |
| | AAUUAGUUAGGGAGGAUUCUCUCUUUUUCUACUCAUUGGAAUAGUUUCAG | |
| | AAGGAAUGGCACCAGAUCCUGUUUGUACCUCAGGUAGAAUUCGGCUGUGA | |
| | AUCCUUCUGGUCCUGGACUUUUUUUGGUUCGUAGGAUAUUAAUUAUUGCC | |
| | UCAAUUUCAGAACCUGUUAUUAGUAUAUUCAGAGAUUCAACUUCUUCCUG | |
| | GUUUAGUCUUAGGAGGGUGUAUGUAUCCAGGAAUUUAUCCAUUUUUUUCU | |
| | AGAUUUUCUGGUUUAUUUGCACAGAGGUGUUUAUAGUAUUCUCUGAUGGU | |
| | AGUUUGUAUUUCUGUGGGAUCAGUGGUGAUAUCCCCUUUAUCAUUUUUUA | |
| | UUGAGUCUAUUUGAUUCUUCUCUCUUUCUUCUUUAUUAGUCUUGCUAGUG | |
| | GUCUAUCAAUUGUGUUGAUCUUUCAGAAAAAAGAGCUCCUGGAUUCACUG | |
| | AAUUUUUGGAGGUUUUUUGGUGUCUCUAUCUCCUUCAGUUCUGCUCUGAU | |
| | CUUAGUUAUUUCUUGUCUUCUGCUAGCUUUUGAAUGUGUUUGCUCUUGCU | |
| | UCUCUAGUUCUUUUAAUUGUGAUAUUAGGGUGUCAAUUUUAGAUCUUUCC | |
| | UGCUUUCUCUUGCGGGCAUUUAGUGCUAUAAAUUUCCCUCUACACACUGC | |
| | UUUAAAUGUGUCCCAGAGAUUCUGGUACGUUGUGUCUUUGUUCUCAUUGG | |
| | UUUCAAAGAAUAUCUUCAUUUCUGCCUUCAUUUCGUUAUUUACCCAGUAG | |
| | UCAUUCAGGAGUAGGUUGUUCAGUUUCCAUGUAGUUGUGUGGUUUUGAGU | |
| | UAGUUUCUUAAUCCUGAGUUCUAGUUUGAGAUGGCACUGUGGUCUGAGAG | |
| | ACAGUUUGUUGUGACUUCUGUUCUUUUACCUUUGCCGAGGACUGCUUUAC | |
| | UUCCAAUUAUGUGGUCAAUUUUAGAAUAAGUGCAAUGUGCUGAGAAGAAU | |
| | GUAUAUUCUGUUGAUUUGGGGUGGAGAGAUCUGUAGAUGUCUAUUAAGUC | |
| | UGCUUGGUUCAGAGCUGAGUUCAAGUCCUGGAUAUCCUUGUUAAACUUCU | |
| | GUCUCAUUGAUCUAAUAUUGACUGUUGGGUGUUAAAGUCUCCCAUUAUUA | |
| | UUUGUGGGAGUCUAAGUCUCUUUGUAGGUCUCUAAGGACUUGCUUUAUGA | |
| | AUCUAGGUGCUCCUGUAUUGGGUGCAUAUAUAUUUAGGAUAGUUAGUUCU | |
| | UGUUGAAUUGACCCCUUUACCAUUAUGUAAUGACCUUCAUUGUCUCUUUU | |
| | GAUCUUUGUUGGUUUAAAGUCUGUUUUAUCAGAGACUAGGAUUGCAACCC | |
| | CUGCUUCUUUUUUUUUUUUCUUUCCAUUUGCUUGGUAGAUCUUCCUCCAU | |
| | CCCUUUAUUUUCAGCCUAUGUGUGUCUCUGCAUGUGAGAUGGGUCUCCUG | |
| | AAAACAGCAAACUGAUGGUUCUUGACUCUUUAUCCAAUUUGCCAGUCUGU | |
| | GUCUUUUAAUUGGGGCAUUUAGCCCAUUUAUAUUUAAGGUUAAUAUGGUU | |
| | AUGUGUGAAUUCAAUCCUGACAUUAUGAUGUUAGUUGGUUAUUUUGCUCG | |
| | UUAUUUGAAGCAGUUUCUUCAUAGCAUUGGUGGUCUUUACAAUUUGGCAU | |
| | GUUUUUGCAGUGGCUGGUACCGGUCGUUCAUUUCCAUAUUUAGUGCUUCC | |
| | UCCAGGAGCUCUUGUAAGGCAGGGCAGGUGGUGACAAAGUCUCUCCACAU | |
| | UUGCUUGUCUGUAAAGGAUUUUAUUUUUCCUUUGCUUAUGAAGCUUAGUU | |
| | UGGCUGGAUAUGAAAUUCUGGGUUGAAAAUUCUUUUCUUUAAGAAUGUUG | |
| | AAUAUUGGCCCCCACUCUCUUCUGGCUGGUAGGGCUUCUGCUGAGAGAAC | |
| | CGCUGUUAGUCUGAUGGGCUUCCCUUUGUGGGUAACCUGACAUUUCUCUC | |
| | UGGCUGCUGGAACAUUUAUUAACACUUACUAAGCUAGGAUUCCACUGAAC | |
| | AAACUUGGAAAAUGGUGCUGUAAGAGGGAUCUGUGCACCCACGAGCAUCC | |
| | UCCACUUGGUAGAGGCCAGGAAGUUCUUUCAAAAGGCCAAUACUCCAGUC | |
| | CACCCCAGACUUCCUGAAUCAGAAUCUUGGGAGGUGGAGUCCAGAAAUAU | |
| | UAGCUUUAACAAGCCUUCCAGACAAUGGUAUGCUGGAGCUGGGAGGUAUC | |
| | AACUCUCCAGAACAGAUUGUUAUUAAAAUUCCAGCAAUUUUAGAACUUCG | |
| | UGAGCUAUUUGACUUCACAUUGGUAGACUGAAAUUUGCUGUGGUAGAAGU | |
| | AUUUACACCAUGAAAAUUGGCAGAUGCUACAAUUCAGGGCUUUUCUGUUC | |
| | UCCCCCAGACAGCUAGUUAUUAGACAUUUAUCCAUAAACCACUGCCUCCA | |
| | GGUCAUCCUUACAUACAUUAAAAUUUGAGACCCACUGAUAAUGCAAGUAU | |
| | GUGCUAUUUCCAGAAGGAAUUAUGUCAAAACUGUUAAAAUUUAAUCCCUA | |
| | GUUUUACCAUGUUUUUAGAUAUUCCUAAAAAAGAUGUGCAGAAAAAAAAU | |
| | CUUACAUUUUCUUUUCAAAAUUAAGUAGAAAUUAUCUAGGAAUAUCUAGA | |
| | AAUAAUCAAGUAAGCAGAACCUCAUUAUGAAGAAGCAAUUUUUUUGUCUU | |
| | UACAGACUUUGGUUGUUGUUUGGGUUUGUUUUUUACAAUGUAUUUUUUAU | |
| | UUUUUAUUUUAGGUGCAGGCAUUUAAUCAUAAAAUUAAUAUUUUGUGCUU | |
| | UGGCAGUUAAAAAUUAUAUAUUUAAGGUGUGUGUAACUUGAUGAUUUGAU | |
| | AUACAUAUACAUUGUGAAAUAAUCACCACAAUCAAGCUAAUUAACGUAUC | |
| | UAUCACCUCACAUAGUUGCCAUGUUUUGUGUGUUGUGUGAUCAGAACAUU | |
| | UUAGACCUCUGUCUCAGCAAAUUUCAAGUGCACAAUAGUAUUCUUCACUA | |
| | AAUUUACAGUGCUAUAUAUUAGAUCUUCAGAACUUAUAUUUUGUAUAACU | |
| | GACUUGAUACCCCUUGAUCAAAAUCUCCCCAUUCUACCCCACCCCACCCC | |
| | AGCCCCUGGCAACCACCAUUCCACUCCCUGAUCCUACAAGUUUGAGUGUU | |
| | UUAGAUAGGUGAGAUCUCAAGUAUUUUUUCUUCUGAGUCUGGCUUAUUUC | |
| | AUUUAGCUUAAUGUCCUCCAGCUUCAUCCAUGUUGUCUCAAUGGCAGGAU | |
| | UUCCCACUUUUUUAAGGUCAAAUAAUAUUUAUUUAUAUAUAUCACAUUUU | |
| | CUUUAUUUUACAGGCUUUCUAAAUUGAGAUAUGUACAGGUCUUGCUGACA | |
| | GAUAUAGCUUUCUUGCUCACAGAUAUAGCUUUACUGUGGUCUAUUCUUUC | |
| | AUAAUCUGGAAAAAAAAUCUAGAAGUAUAAAAAUAUUCUCGCCAGGCGUG | |
| | GUGGCUCACACCUGUAAUCCCAGCACUUUGGGAGGCUGAGGUGGGCAGAU | |
| | CACGAGGUUAGGAGAUCGAGACCAUCCUGGUUAACACAGUGAAACGCUGU | |
| | CUCUACUAAAAAAAAUACAAAAAUUAGCCAGGCGUGGUGGCAGGCACCUG | |
| | UAGUCCCAGCUACUCGGGAGGCUGAGGCAGGAGAAUGACGUGAACCCAGA | |
| | AGGCGGAGCUUGUAGUGAGCUGAGAUUGUGCCACUGCACUCCAGCCUGGG | |
| | CGACAGACCAAGACUCCUUCUCAGCAAAAAAUAAAAUAAAAAUAAAAAAA | |
| | UAAAAAUUUUCUCAGAUUGUUGUCACUUUUUAAAAAAACUUAUAUACUCC | |
| | UCUUCGCCACCACCUUUCAUUGAAAAGAAACACAUCUCACACCUGUUCAG | |
| | AUUUUAGCUGGGCUCUUUUGUCCCACUGGUGCUUUAGGGGUAUUGAUAUA | |
| | GUACAUUAUUUCUUUCUUAAAGGGGAUGCCUUGGACUGAUCCAAUUAGAG | |
| | UCCUGAUCUCACAUUUGAUAGCAAACACAGAGCCCAGAAUGAUCUUUUCU | |
| | UUAUUUCUCUGGUUCAAGAAAAGUACAGAAUCCAAUGACAGAAGCAGAAA | |
| | AUAGAUUGUGAGAUGGAAGAGUCUGAGUUUCCACUUAACAUUUGUCCUUC | |
| | UGUUCCCUCUGAAGGUGGACAACAUGAAAGUGACUGCUGGACUAAAAAUG | |
| | GUUUUGCUUGAACAGCUUAACUGACAUAAACAUACGCCACUGAGAGUCAU | |
| | AUCCCAGGUCACUAGAACCUUGACCCACCACCAAGAUGUAUUUAAUUUUU | |
| | GUGAAUCCAGCAGUUAAUUCUGACAGCCAAAUGCUACCAGGUUGUUUAUC | |
| | UCUCUUGUAAAAUGCCAUAUGUUAUAUGCUUAGAGGCAAGCAUCUCAUGU | |
| | CUCCAUUUUAAAAUUAACAAGUUAAUUUUUAUAGCCCAAAUCUUAAGAAU | |
| | UUGAGCAUCAGAUGCCUUUAAAAAUUUGGUAUAUUUUUAUGUCAUACUUU | |
| | UAAAUUAUUUCCAUCUAAUAGUGUUUGCUCCAUAAACAAAAUAUGUCACU | |
| | UAUAACUUUAUAUUUUUGUAAGUUUUUAUGUAAGUUAAAGAAAAUUAAAU | |
| | AUCUUGUAUUACAUACUAAAUUAGAAAAAAAAACCUAUUGUUAUAUAGUU | |
| | GAACAUUGAGUUAUUUAAAAUUAACAUCAAAAUACAGUAUCUGAAUGUUU | |
| | UUACAAAAAAUUCUUAUCAUUUACACAAAAUAAUAAUUUCUUUUAAUUUU | |
| | UAAGGAGAUUUGAAAAGAGUUUAGAAUUUGAAAACAUUUUGCUAAUUCAG | |
| | CCUUCAAGUAAAUUCAUGUAACAUGGAAUAUACACACUUAAAAGCUGGCU | |
| | ACACCUAAAUAAAAUUCCAAAGAAAUCUUUCCGAAAUGAUAGAGUCGCUG | |
| | ACCCAUAAUAGGGAAAUGUUUGGGGUCAUUGAAACCUUUAUCAAAUUGUU | |
| | UUAAUCUAACUUCAUGAUUACCUACUUGCCCAGCAAUGUAGAUUAUUGUG | |
| | UUUUGCAGGAUGUUAACAUUGAAGUUUGAACUGAGGUACCCACCCUUACA | |
| | AAUAUAGGAUGUCAAAAUGAAUACAACAUACUGCAUAUGUACAAGAUUUA | |
| | CAAAGAAUUAUCGAACUUAGCUUAUGAUGUUUAGAUGCUCAGCCAGAUUA | |
| | AGGAUAUACAAAGGUGAAGGGUUUAAAAGAAUUUUUAGAGAGUUAUUGCC | |
| | AUACUAUGAAUGUCUGUUUCCACCCCUAUCCUUAAGGCUCCACCUUGAGC | |
| | UUAACAGGCCUGUGGACAGGUCAUCUGUACUUCUACAGCGAGGGCAAGGG | |
| | AUACAUGAAUGGUUCCCUUGGACAGAGCGGGCCUUGAGAGAAAUCCAAUG | |
| | GAGGUCAACCUAGGACUUCCCAAAGGGCUAGAAGGAAUCUAUCAACAAGA | |
| | AGCUGGAAGGGAAAAUUGGAUGUCUACUCAUUCCAGAAAGAGUUGGAAAU | |
| | GACCAUACAAAUUCUCAAGGAGAGGUCAGCAAUAUUUCUCUGGAAAGGGU | |
| | CAGAUAGUAAAUAUUUCAGGCUUUGAGGGCUGUACCGCCUUUGUAGCAAC | |
| | UACUCAACUGUUUGGUUAUUGCACAAAAGCAGCCAUAAGCAAUGUGUAAA | |
| | CAAAUGGAUGUGUCUAUGUGCAGGUAAAACUAUUGACAAAAACAGGUGGC | |
| | AGGUUGGAUGGAGCCUACAGGCCAUACUUUAUCAACCCUGCUAUAGAUGC | |
| | AUCAGCUUGAGACAAAGGAGGAGGAGGUGAGCAGAGCAGAGCCCAGCUAU | |
| | GGAAAAGCCCAUGAAGGUCAAGUUCACUUCAACAUCCUGCCAGGUCUGCA | |
| | GCAUGUUCACACGACAGCCUUCCCCAUUCCUCAUUCCUCUUCCCUCCCUG | |
| | GCACACCUAUCCUGGAGACAUUAGAAGCUGGCCAGCGAGCUGGUUUGAGA | |
| | CAGGAGGAAAAGCCAGAGAAAGAGAAAUAAAGAAGCAGCAGCCACCAACC | |
| | CUGCACCCUUUCUCCCCAGCAAGCUUCCAGCCUGGGGAAGACAGCCAAGA | |
| | GAGGAGCGAAUAUUUGACAUUAACUCCAAUUCAGAGAUCCUGGACUGAAC | |
| | ACUCUUAAUUUCUGAAUGGAGACAAUUUCCAACUUAAAGUGACCAGAGAU | |
| | AGAACUGUUUAUUCCCUAAGAAUCAGUAGAAAAGUCACAGGACAGAGGAA | |
| | GGGAAAAGGAAAGCUAUAUUUUUAUUUUUAUUUACUUAUUUUUCACCCUG | |
| | UCUUAUGAUGCUGAGCUAUAUUUUUAUUAAAUCACACUUGCUUUUUCUAA | |
| | AUACUGGUUACAUACACAAGCCCAGAGUCAAACAUUUUCUUACCCUAUCU | |
| | GAAGACUCCAGCAGUAACAGAGAAACAAAUAACGAUGACAUCCACCUCAG | |
| | CCACGUCUGGCAAACUUGACACAAGCGGCUCACCAAGAAGGCAACCCAGC | |
| | AGUUAAUGUUACUGACCCAUUUGUCAUAUCCAGUUUCUCCAACUGUCCAU | |
| | AACAGGUAUUUAAUUAUAGAGGAUGAUUGGGGAUUCAUUUGUCCUUCAGG | |
| | AAAUUUCACGACUAAGGUAUGUCUUUCUUCCAGUAACAUGCACACACUCA | |
| | CACAGACUCAUUUAAUUCUCAAACCUAACAUGAUUAGGUUGGUCUAGGAA | |
| | AGGCCUCUCUGAGAAGGUGAAUUAAAGUUGAUCUGGACGAUGAGAGACAG | |
| | UCACGCAGAGAUCAACAAGGAGUACAUUCCAGAGAGGGGAAAAUGUUCAU | |
| | GCAAAGGACCUAUGGUAGGAACAAAUGUCAGGGAGCAGAAAGAAGCUUGG | |
| | UGUGUCUGAAGCCUGUGCAUGAUGUGAGGAUGAUAAGAGGUAUGUACAGA | |
| | GAGGCAGGUGAGGGUCAUGUGCUGAGGGCUUUUCAAGAAAGGACUUUGCU | |
| | GCAUUUGGAUUUUAUUCUAAGUUUGACAAAAAGACAGAUGAUGGGGGGCA | |
| | UUAAAUCAGGAGAAUAACAUAAACAUGACCUGACCUACAUUUUUAAUAUU | |
| | GUGUUCUGGCUUCUUUAGGAAGAAUGAAUUGUUAGGAGAUAAUAAUGGAA | |
| | AAUCAAUACAGAAACCAUUAGAGUCCUCUCGGCAAGAGAUGGUGGUGUAC | |
| | UUGCCUUAGGAUAGUAGUAAAGAUGAAGAAAAGUGGAAGAAUUCGGGGCA | |
| | GACUUUGAAAUAGAAUUGGCAGGACAUGCUGAGAGAUGGAUGUGGGAAUA | |
| | AGGGAAUAGAGGGAAUGAGAAUAAUGUCUAGAUUUGUGGCUUGAGCAACU | |
| | GGGUGGUGGUGUGAGGCCACUUACUGAAAUGGAGUCUGCUGAAUAGAAAC | |
| | AGAUGGGGAGGUUGGUAAAGAAUAAAGAUUGUCCUUAAUCGUGUUAGGUU | |
| | UGAGAUACCAUUUGGACACCCAAGUGGAGAUAUCAGGUAGGCAGAUAGAC | |
| | UUGGGCAAGUCAGUGCUGGAAAGCAAUAUUUGGGGUUAUCAACAUAAAAA | |
| | UGCUACUUGAUGCCAUAAGAAUAAAUGAGGUCAUCAAGGGAGAGAAGAAC | |
| | AGAAAGAAAAGUGAAGAAAAAAAUCCCAAGUCAUAAGCCCUGGGAUAUUC | |
| | CAUUGUUCAGAAUAAUAAGAAGAAAGAGGAUCCAUCAAACUAGUCUGCAA | |
| | AAAAGCAGCCUGAGAGGUGGAAAAUAAACCAAGCAAGUGUAGUGCCACAA | |
| | AAGCCAGAAGACCAAAGUGUUCUGUAAAGAAAGGAAUAGCCAACUGCACC | |
| | UAAUGCUGUAGAGAUGCCAGGAAAGGGGAGGAAGUGAUGUGACUGUAAUC | |
| | AGCAGCCUAGAGGUCAUCGGUGGGCAAUUCAAGAACAGUCUCAAUGGAGG | |
| | UGGGAUCACAAGUUCAGUAAAAUGGGGAUGAAGUCAUUAACUACAGAAAA | |
| | GCAACUUAAGACAUAUCACCAAGACAAGUAGCAGAGUAAUAAGGAAGUAG | |
| | CUAUUAAUAAAAAAAAAUUUAUCAGCCAAGUAUGAAAUGCAAGGCAUUGU | |
| | ACUAAGUGCAAUUGGAGAAUAGGAGAGAAACACUUUACAUUCCAUGCUCU | |
| | AUAGAGCUCAAAAAGUAUGGCAAGGCAUUGGAAGACUAAUCAUGGUGACA | |
| | UGUAUUUAUUAGCUAUUGCCAUACUAACACUAUAUAACAAAUAGUCCCCA | |
| | CAAUACAAUACACAAUAAGGAUUAAUCUCAAACACAAUAAGGAUUGAUUU | |
| | UUGCUUGUAAGUCAGCUGGAAGGUUCUGCUGAUUGGAGUCUGGCUCAGGG | |
| | AGUAGGAGCUGGGCUUGUUCAAGUGUCUGUACUCAGGGCCUUUCUCGGUG | |
| | CCAGAACCUUGGUGAGUGCAAAAGUUGCUUGUCCAUGAGUAUGGCUCUAC | |
| | CCACCAAAUUGGAAGAACACGAUCUGCCAAUGAAGAGAAUGAGGCUAGCA | |
| | UAUAAAGAGGGAUGAAGAUAAUUAUUAAGGAGUCUUAACUAUACCUUUGA | |
| | GCUUGAAUCCAAAUAUGCCUGAAGUCUUCCAUCCUGCGCUUCCCAGUAAC | |
| | GGAAACUAAAAACCUCCCAUUGUGUUUAAUAUAGUCUUAAUUGGACUCUG | |
| | CCACUUGCAAUCCAAAGAGUUCUGACCAAUACAUCCAAGUUAAUUUUGGA | |
| | CCUUUAAAAAAAUAAAUAUCCAUUGAUGAUUUAAUCAAAGAGAGAUUAUU | |
| | UUAAGGGGGAAUUUAGGAAGAUUCAUUUUCACAAACUGGUUAGGAGAGAU | |
| | AAUAAACUGCUGAGAUGAAGUCCAGCUGGCAAGAAUUUCAUAAUUUAGUU | |
| | CCUGAGGAUAAAAGGAAGGAAAAUCCCAUUUUCCUGUACCACAGCUGAUA | |
| | UUCAGUUGGUUCACAUAGGUAUAGCCAUGCUGGUUAUUUAAUGCCAGCAU | |
| | CCAUUCUGAUAAGUGGUAUCCACAUCUUCUGAUUAUUAGGUAUCUUGAUU | |
| | AUCACUGCUGUCCAUAUCUAUGCUGCAAUAUAUGUUCAACAAAGUGAAGC | |
| | UGCUUAGGAAUAUUUUAGAAUUCUGUUUUCUCUCUACCUAUGAUGUGUAA | |
| | CCUCAAUUUCUUUCACCUUGGAUAUCAAGAUGCAACAGAAUAAAUUUGUC | |
| | UGCAGCCUGGAUGAACUUGCUCCCCACGAGAGGGACUGACUUUACUCAUC | |
| | CUUCUCUUUUAACCCAGUCAUGCUGAAAUUAUCAGAGAGAGCCAGGCAGA | |
| | AUGAAAAUGGCAGUAACAUCCAGACUCUCCUAGACAUUGACCGGAAGUUG | |
| | ACCUCUGGCCACAGAUGCCAGAAGCAGGCAGGCAAGGAAGGGACCAAGGA | |
| | GAAGCCAUCAAGAAAAUGAAGGUACGAUAAACUGUGAAAAUUGCGUUGGC | |
| | AAUGCAUGAGUUACCACUGAGUCAACCUAUAUUUCCAACAAAAUCCUGGA | |
| | GAAUAAGCCUGCAGAAUACACAGACUAAAAAGAUGGCGGCUCCCCACCCC | |
| | AAAACACCUUCCCUACCCACCAGCCUCUGCUCAAAAUACCACCACACAAA | |
| | CUUGAUUUUUUAAUCUAAGGAAAGAAUCAAGCAGAUGAACUAUGAUUGAA | |
| | GCUUAUUUUGUAAUAGCAAUGAUUCAAAAACAAACUCUAUGUCCAAACUU | |
| | AGUCAGCUUUCCCUGACUGGCUAAAUAAAUUAUGAUGUGCCCAUACAAUG | |
| | GUGAGGCAGGAGAAUAGGGUCUGGAGGCAGAGAACCUAAGAUCGAUUCAC | |
| | AUUGACUUCUUAGGACUAAAUCAAAAGGAAAACCCCAACUUUCCACAACU | |
| | AAGAAACAAAAGUGUGGAAGCCUACUUCCUUUGCAAACCCUCCACCCACU | |
| | UUUUCUGCCUGGCAGAUGGAAAAUUGAACAUAUCUCUGAUUGGUUGCAGA | |
| | AAGCAUAGGAGUGUAACUUUUGUAACUUUACUUCAGCCUCUGAUGGGUUG | |
| | CUUUCCACAACCAUUCAGACACUUGCAUAGAAUGUAACCUUUGUAACUUC | |
| | ACGUCAGCCUCUGAUUGUGGGACCCACUUCAUUUACCUAGGGUGUACACA | |
| | AAGGAACCAGUGGGAAACCUCUAGAGGGUAUUUAAACCCCAGACAAUUCU | |
| | GGAACCAGGCUUUUGAGCCCCUAUGCUCGGCCAGCACCCACCCUGUGGAG | |
| | UAUACAUUCAUUUUCAAUAAAUCUUUGCUUUUGUUGCUUCAUUCUUUCCU | |
| | UGCUUUGUUUGUGUGUUUUGUCCAAUUCUUUGUUCAAGAUGCCAAGAACC | |
| | UGGACACCCUCCACCAGUAACAAUGGAAUACUGUAAUUCCAUUGAGCCCA | |
| | GACAGUGUCACGUGCCACCCCUACCUGCAUGGGAUCCUGGGGAGGCUAAU | |
| | AGGUUUAGUUGAGAAUAUUACCAUCUUGAACAAAGUCAGAGUUCUCAUCA | |
| | GAAAGAAGUAGAAAUGACAACUUGCUGCAUCUGCCACGCAACUUCAAUAU | |
| | CCUGCAAUAUGAGAUUGUUUGAGAAACGAAUGUACAACCACAUGAUGGAA | |
| | UUCUCUACAGAUGUCUAAUGCUAGAGAAAGAAUAAAGGGGAAAAUUGUGU | |
| | GGUAAAUGUUUUAGACAUUUUAAAUGAAAAAAGCAUUUUCAUUUAAAAAA | |
| | UGAAAAAGUUGAAAAAAUGUAAAUAAUAUGAUCACGUGUGUGUAUAUGUU | |
| | UGCGCGUGGAUGGGUGCGUGUGUAUCUCCUAGUACAUGCAUAGAAAAUCU | |
| | GUGGAAGAGGCUGGGCGCGGUAGCUGAUGCCUGUAACCCCAGCACUUUGG | |
| | GAGGCCGAGGCAGGCAGAUCACGAGGUCAGGAGAUCGAGACCAUCCUGGC | |
| | UAACACUGUGAAACCCCGCCUCUACUAAAAAUACAAAAAAAUUAGCCGGG | |
| | CAUGGUAGCACGCACCUGUAGUCCCAGCUACUCGGGAGGCUGAGGCAGGA | |
| | GAAUCGCUUGAACCCAGGAAGCAGAGGUUGCAGUGAGCUGAGAUUGCACC | |
| | ACCGUACUCAAGCCUGGGCUACAGAGCGAAAUUCCAUCUCAAAACAAACA | |
| | AACAAACAAUUACAAAAAUUAGCCAGGAGUGGUGGUGGGUGCCUAUAAUC | |
| | CCAGCUACUCAGGAGGCUUAGGCAGGAGAAUCGCUUGAAUCCAGGAGGCG | |
| | GAGGUUGCAGUGAGCCGAGAUCCUGCCACUGCGCUCCAGCCUGGCCACAG | |
| | AGCAAGACUCCAUCUCAGAAAAAAAAAAAAGAAAGAAAGAAAAGAAAAUC | |
| | UGUAGAAGAAUACGUAAGAAAGCACUAUUGGUAGUUAUUUCUGGAAGAUA | |
| | AGACUGAGAAGGAACAUGACAUUUACUUUUCACUUCAAUCCAUAGUAUUU | |
| | CUCUUGUCAUUAGAAUCAGAGAAUUUAUUGGUAUAAAAAUACCCAGGACU | |
| | CCUACCGUGGUAAUUUGAAAAUUUGUUUCCAGAUAGUCUAAAUAUAAAAC | |
| | ACUCUGGAAAAAGCUGCCUCAAUCCUGUCCCCUGGGACCCUCAGCAAGGG | |
| | GCCUGUUAACCACGAUUCCACUUCAGCAAAGAAGCCCCACCAGAAGGAAA | |
| | AAACCACGCACCAGCCAGGACUCUACGGGAGAGAGAAGCCCACCAAAGGC | |
| | UUUAGAGGAUAACAUCGCAGUUGUUCUCCAUGUUACUUUUUUCUCCAAGG | |
| | UAAGGAAUACACUCCUAAAUGUUCCUAAACUCAUUUUGCUAAUCUGAUUU | |
| | AUCAUAAUAACCCUCAAUAAAAGUCCUCCUUUGUAUUUAAGUUAAAACUC | |
| | UUUAUGUAACCUAUAAUUUUAGUUCUAUCAGGCUCACAGAAAGUUGGUCA | |
| | CCAUCCUCCCUAUUGUAAUUAAACACUCAAAUGCCUCCUAACGACUAAAG | |
| | AAAUUAAACCUAUAUAUCUAGGACCCAGGAGCCAAAACCGUAACAGCUCA | |
| | GACAAAAGCUUGAGUUUUUUUAAGUUUGUAUUUUAUAUUAACAAUUUUUU | |
| | AGCAUUUUACACUCUACUCCAUGAUAUAGCUAUAACUGAUUCCCACUAAA | |
| | AUAAUUUCUUCCUCUCCCCUCUCUUCUGGAAUAUUUGAAUAAAUUGAUAU | |
| | UCCAGGAAGAUGUGUCUUGUUUUCGCAAAGGCCACCCCCAACAACAUAUU | |
| | CCCUCAGAUUAUGCUUUCAUAUGACCUCAUCCCUCAGGCUUUUCUACUUU | |
| | UAAGUCAACCAUUUCUAAUUCUCUUAACCCAUAGACAUAGGUCCCAAUUU | |
| | UUCAAUUCUUCAAAUUUCCCCAUCAUUUUCCUCCAAAGCCUAUUCCACUU | |
| | AUCACUAACGCUCAAAUAAUGUGCUCUAAAAUUCACUGCAGUGCUCCAAU | |
| | GAGAUGCUGUUGGAAAGGGUCUCAAUUUAUGCUCCCAUUUUCUCUCCCCU | |
| | CAUAUUUGUACACCCACUGUUUAUUUGUACCUCUGUACUUCCUGGAAGGA | |
| | AGAACACAUUUAAUAAUCAAGCCAACUAGUAUUGCUGAGGCUUUCUACUA | |
| | AAAACUUGUAGAUCAUUGCAUAGAGCACAGCAGGACUGAUUAGUAUGCCA | |
| | GGGUGAAGACAUUGAAAAAAUAAACUAGAAAAGUCCAAGGGAAGUUGUGA | |
| | UAGACUGUGUGAAGUUUAGAAUAGAAAAUGGGUUCAUAAAUUAAGGAGAG | |
| | ACGUGGGAAAUUUUGCAAAUAUAUGCAUAAAGUGAACAGCCAAACGGUGA | |
| | GUUUCACCCAGUGUAACACCCAGCAUCUGUCCCAAUUUGAAGCCCUUACC | |
| | UUUAAGCAGGCCGGCCUGCCCAGCAGUUCACAUAGCCUCAGCGAGAGGGC | |
| | AUGCCAAGAAGUCUGCUUUGUUCCCCGGCCAAUGCUGGACCUUCCUGCCG | |
| | AGACAUGCCUCACACUCAUAUUUCCCAGCCUGUUCUAAUGACGCCAGCUC | |
| | UCAACUUCUCAGCUGCUGUCUCUGACUUUGCUCCUCUGUCCAAUGCCUGA | |
| | UAUUAUUGUAUCUCUGGGAUUAAGCCUUGGUUCCCUGAAAUUGCAUUCAC | |
| | CACAAACUACAUGUGCUUUUAGCUGCUCCAGAGAACAUCCUAGCAUAAUC | |
| | UGGCACAUCCUCUGGACAAAACCCUGGUUGGACCUAGCCAAUAAGACAGA | |
| | AGUUACUGCUGGGAUAAUCCCAGUUGCUGAAGAGGAAAACUAUGUUUAUC | |
| | UAUGCAUACUAAUAUGACUGAAAAAAUAUUUUCUUUAUUAAUCUAUGGAG | |
| | AAAAAUGUCCCUAACACAGUCUGGGACAAAAAUCACUUUACAAGGUUCUU | |
| | UACAACCUUCU | |

In some instances, one or more transposable elements are associated with conversion, generation, development or activity of CAF. In some instances, one or more transposable elements are associated with conversion, generation, development or activity of myCAF. In some instances, the transposable element regulates the differential expression of a RNA, a protein, or an element of a signaling pathway related to the initiation, development or function of CAF. In some instances, the transposable element regulates the differential expression of a RNA, a protein, or an element of a signaling pathway related to the initiation, development or function of myCAF. In some instances, one or more transposable elements are associated with the expression or activity of lncRNA disclosed herein. In some instances, the transposable element regulates the differential expression of the lncRNA disclosed herein. In some instances, the expression or activity level of the lncRNA disclosed herein is altered by change in expression level of the transposable element. In some instances, one or more transposable elements are within a genomic locus encoding the lncRNA disclosed herein.

In some instances, one or more enhancers (e.g., super-enhancers, binding sites of master transcription factors) are within a genomic locus encoding the lncRNA disclosed herein. In some instances, one or more super-enhancers (e.g., primary CAF super-enhancer) are within a genomic locus encoding the lncRNA disclosed herein. In some instances, one or more binding sites of transcription factors are within a genomic locus encoding the lncRNA. In some instances, the one or more enhancers comprise super-enhancers that are associated with the conversion, generation, development or activity of CAF. In some instances, the one or more enhancers comprise binding sites of master transcription factors that are associated with the conversion, generation, development or activity of CAF. In some instances, the master transcription factor comprises paired related homeobox 1 (PRRX1).

In some instances, one or more enhancers (e.g., super-enhancers, binding sites of master transcription factors) are located at most 10 nucleotides, at most 20 nucleotides, at most 30 nucleotides, at most 40 nucleotides, at most 50 nucleotides, at most 55 nucleotides, at most 60 nucleotides, at most 65 nucleotides, at most 70 nucleotides, at most 75 nucleotides, at most 80 nucleotides, at most 85 nucleotides, at most 90 nucleotides, at most 95 nucleotides, or at most 1000 nucleotides upstream from the genomic locus transcribing lncRNA disclosed herein. In some instances, one or more enhancers (e.g., super-enhancers, binding sites of master transcription factors) are located at least 10 nucleotides, at least 20 nucleotides, at least 30 nucleotides, at least 40 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides at least 90 nucleotides, at least 95 nucleotides, at least 1000 nucleotides or more upstream from the genomic locus transcribing lncRNA disclosed herein. In some instances, one or more super-enhancers (e.g., primary CAF super-enhancer) are located at most 70 nucleotides, at most 75 nucleotides, at most 80 nucleotides, at most 85 nucleotides, at most 90 nucleotides, at most 95 nucleotides, at most 100 nucleotides, at most 150 nucleotides, at most 200 nucleotides, at most 250 nucleotides, at most 300 nucleotides, at most 350 nucleotides, at most 400 nucleotides, at most 450 nucleotides, at most 500 nucleotides, at most 550 nucleotides, at most 600 nucleotides, at most 650 nucleotides, at most 700 nucleotides, at most 750 nucleotides, at most 800 nucleotides, at most 850 nucleotides, at most 900 nucleotides, at most 950 nucleotides, or at most 1000 nucleotides upstream from the genomic locus transcribing lncRNA disclosed herein. In some instances, one or more super-enhancers (e.g., primary CAF super-enhancer) are located at least 10 nucleotides, at least 20 nucleotides, at least 30 nucleotides, at least 40 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 1000 nucleotides or more upstream from the genomic locus transcribing lncRNA disclosed herein. In some instances, one or more binding sites of transcription factors are located at most 70 nucleotides, at most 75 nucleotides, at most 80 nucleotides, at most 85 nucleotides, at most 90 nucleotides, at most 95 nucleotides, at most 100 nucleotides, at most 150 nucleotides, at most 200 nucleotides, at most 250 nucleotides, at most 300 nucleotides, at most 350 nucleotides, at most 400 nucleotides, at most 450 nucleotides, at most 500 nucleotides, at most 550 nucleotides, at most 600 nucleotides, at most 650 nucleotides, at most 700 nucleotides, at most 750 nucleotides, at most 800 nucleotides, at most 850 nucleotides, at most 900 nucleotides, at most 950 nucleotides, or at most 1000 nucleotides upstream from the genomic locus transcribing lncRNA disclosed herein. In some instances, one or more binding sites of transcription factors are located at least 10 nucleotides, at least 20 nucleotides, at least 30 nucleotides, at least 40 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 1000 nucleotides or more upstream from the genomic locus transcribing lncRNA disclosed herein.

In some instances, one or more enhancers (e.g., super-enhancers, binding sites of master transcription factors) are located at most 70 nucleotides, at most 75 nucleotides, at most about 80 nucleotides, at most 85 nucleotides, at most 90 nucleotides, at most 95 nucleotides, at most 100 nucleotides, at most 150 nucleotides, at most 200 nucleotides, at most 250 nucleotides, at most 300 nucleotides, at most 350 nucleotides, at most 400 nucleotides, at most 450 nucleotides, at most 500 nucleotides, at most 550 nucleotides, at most 600 nucleotides, at most 650 nucleotides, at most 700 nucleotides, at most 750 nucleotides, at most 800 nucleotides, at most 850 nucleotides, at most 900 nucleotides, at most 950 nucleotides, or at most 1000 nucleotides downstream from the genomic locus transcribing lncRNA disclosed herein. In some instances, one or more enhancers (e.g., super-enhancers, binding sites of master transcription factors) are located at least 10 nucleotides, at least 20 nucleotides, at least 30 nucleotides, at least 40 nucleotides, at least 50 nucleotides, at least 55 nucleotides, at least 60 nucleotides, at least 65 nucleotides, at least 70 nucleotides, at least 75 nucleotides, at least about 80 nucleotides, at least 85 nucleotides, at least 90 nucleotides, at least 95 nucleotides, at least 1000 nucleotides or more downstream from the genomic locus transcribing lncRNA disclosed herein. In some instances, one or more super-enhancers (e.g., primary CAF super-enhancer) are located at most 70 nucleotides, at most 75 nucleotides, at most 80 nucleotides, at most 85 nucleotides, at most 90 nucleotides, at most 95 nucleotides, at most 100 nucleotides, at most 150 nucleotides, at most 200 nucleotides, at most 250 nucleotides, at most 300 nucleotides, at most 350 nucleotides, at most 400 nucleotides, at most 450 nucleotides, at most 500 nucleotides, at most 550 nucleotides, at most 600 nucleotides, at most 650 nucleotides, at most 700 nucleotides, at most 750 nucleotides, at most 800 nucleotides, at most 850 nucleotides, at most 900 nucleotides, at most 950 nucleotides, or at most 1000 nucleotides downstream from the genomic locus transcribing lncRNA disclosed herein. In some instances, one or more super-enhancers (e.g., primary CAF super-enhancer) are located at least 10 nucleotides, at least 20 nucleotides, at least 30 nucleotides, at least 40 nucleotides, at least 50 nucleotides, at least 55 nucleotides, at least 60 nucleotides, at least 65 nucleotides, at least 70 nucleotides, at least 75 nucleotides, at least about 80 nucleotides, at least 85 nucleotides, at least 90 nucleotides, at least 95 nucleotides, at least 1000 nucleotides or more downstream from the genomic locus transcribing lncRNA disclosed herein. In some instances, one or more binding sites of transcription factors are located at most 70 nucleotides, at most 75 nucleotides, at most 80 nucleotides, at most 85 nucleotides, at most 90 nucleotides, at most 95 nucleotides, at most 100 nucleotides, at most 150 nucleotides, at most 200 nucleotides, at most 250 nucleotides, at most 300 nucleotides, at most 350 nucleotides, at most 400 nucleotides, at most 450 nucleotides, at most 500 nucleotides, at most 550 nucleotides, at most 600 nucleotides, at most 650 nucleotides, at most 700 nucleotides, at most 750 nucleotides, at most 800 nucleotides, at most 850 nucleotides, at most 900 nucleotides, at most 950 nucleotides, or at most 1000 nucleotides downstream from the genomic locus transcribing lncRNA disclosed herein. In some instances, one or more binding sites of transcription factors are located at least 10 nucleotides, at least 20 nucleotides, at least 30 nucleotides, at least 40 nucleotides, at least 50 nucleotides, at least 55 nucleotides, at least 60 nucleotides, at least 65 nucleotides, at least 70 nucleotides, at least 75 nucleotides, at least about 80 nucleotides, at least 85 nucleotides, at least 90 nucleotides, at least 95 nucleotides, at least 1000 nucleotides or more downstream from the genomic locus transcribing lncRNA disclosed herein.

In some instances, one or more protein-coding genes are located within the genomic locus encoding the lncRNA disclosed herein. In some instances, one or more protein-coding genes that are associated with conversion, generation, development, activity, or function of the CAF are located within the genomic locus encoding the lncRNA disclosed herein. In some instances, one or more protein-coding genes that are associated with a pro-tumorigenic or fibrosis development function of the CAF are located within the genomic locus encoding the lncRNA disclosed herein. In some instances, one or more protein-coding genes of LRRC15 (NM_001135057), COL12A1 (NM_004370), DYRK2 (NM 003583), FN1 (NM 212482), or CAPN9 (NM_006615) are located within the genomic locus encoding the lncRNA disclosed herein. In some instances, one or more protein-coding genes of LRRC15 (NM_001135057), COL12A1 (NM_004370), DYRK2 (NM 003583), FN1 (NM_212482), or CAPN9 (NM 006615) are located upstream of the lncRNA disclosed herein. In some instances, one or more protein-coding genes of LRRC15 (NM 001135057), COL12A1 (NM_004370), DYRK2 (NM_003583), FN1 (NM 212482), or CAPN9 (NM_006615) are downstream of the lncRNA disclosed herein.

In some instances, one or more protein-coding genes that are associated with conversion, generation, development, activity, or function of the CAF are located at most 70 nucleotides, at most 75 nucleotides, at most 80 nucleotides, at most 85 nucleotides, at most 90 nucleotides, at most 95 nucleotides, at most 100 nucleotides, at most 150 nucleotides, at most 200 nucleotides, at most 250 nucleotides, at most 300 nucleotides, at most 350 nucleotides, at most 400 nucleotides, at most 450 nucleotides, at most 500 nucleotides, at most 550 nucleotides, at most 600 nucleotides, at most 650 nucleotides, at most 700 nucleotides, at most 750 nucleotides, at most 800 nucleotides, at most 850 nucleotides, at most 900 nucleotides, at most 950 nucleotides, or at most 1000 nucleotides upstream from the genomic locus transcribing lncRNA disclosed herein. In some instances, one or more protein-coding genes that are associated with the CAF are located at least 10 nucleotides, at least 20 nucleotides, at least 30 nucleotides, at least 40 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 1000 nucleotides or more upstream from the genomic locus transcribing lncRNA disclosed herein. In some instances, one or more protein-coding genes that are associated with a pro-tumorigenic or fibrosis development function of the CAF are located at most 10 nucleotides, at most 20 nucleotides, at most 30 nucleotides, at most 40 nucleotides, at most 50 nucleotides, at most 55 nucleotides, at most 60 nucleotides, at most 65 nucleotides, at most 70 nucleotides, at most 75 nucleotides, at most 80 nucleotides, at most 85 nucleotides, at most 90 nucleotides, at most 95 nucleotides, or at most 1000 nucleotides upstream of the lncRNA disclosed herein. In some instances, one or more protein-coding genes that are associated with a pro-tumorigenic or fibrosis development function of the CAF are located at least 10 nucleotides, at least 20 nucleotides, at least 30 nucleotides, at least 40 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 1000 nucleotides or more upstream from the genomic locus transcribing lncRNA disclosed herein.

In some instances, one or more protein-coding genes that are associated with the CAF are located at most 10 nucleotides, at most 20 nucleotides, at most 30 nucleotides, at most 40 nucleotides, at most 50 nucleotides, at most 55 nucleotides, at most 60 nucleotides, at most 65 nucleotides, at most 70 nucleotides, at most 75 nucleotides, at most 80 nucleotides, at most 85 nucleotides, at most 90 nucleotides, at most 95 nucleotides, or at most 1000 nucleotides downstream of the lncRNA disclosed herein. In some instances, one or more protein-coding genes that are associated with the CAF are located at least 10 nucleotides, at least 20 nucleotides, at least 30 nucleotides, at least 40 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 1000 nucleotides or more downstream from the genomic locus transcribing lncRNA disclosed herein. In some instances, one or more protein-coding genes that are associated with a pro-tumorigenic or fibrosis development function of the CAF are located at most 10 nucleotides, at most 20 nucleotides, at most 30 nucleotides, at most 40 nucleotides, at most 50 nucleotides, at most 55 nucleotides, at most 60 nucleotides, at most 65 nucleotides, at most 70 nucleotides, at most 75 nucleotides, at most 80 nucleotides, at most 85 nucleotides, at most 90 nucleotides, at most 95 nucleotides, or at most 1000 nucleotides downstream from the genomic locus transcribing lncRNA disclosed herein. In some instances, one or more protein-coding genes that are associated with a pro-tumorigenic or fibrosis development function of the CAF are located at least 10 nucleotides, at least 20 nucleotides, at least 30 nucleotides, at least 40 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 1000 nucleotides or more downstream from the genomic locus transcribing lncRNA disclosed herein.

In some instances, epigenetic modifications or changes are present within the genomic locus of the lncRNA disclosed herein. In some instances, epigenetic modifications or changes comprises H3K27ac, H3K4me3, H3K4me1, H4K16ac, H3K27me3, H3K79me2, H3K36me3, H2AFZ, H3K9ac, H3K4me2, H4K20me1, H2BK120ac, H3K56ac, H2AK9ac, H3K18ac, H4K5ac, H2AK5ac, H3K9me1, H3K4ac, H2BK5ac, H3K14ac, H3K79me1, H3K23ac, H2BK15ac, H3K4me2, H2BK12ac, H4K91ac, H4K20me1, H2BK20ac, H4K8ac, or a combination thereof. In some instances, H3K27ac modifications are present within the genomic locus encoding the lncRNA disclosed herein. In some instances, such epigenetic modifications or changes can be used as a marker to identify the genomic locations transcribing the lncRNAs as described herein.

In some instances, the lncRNA disclosed herein modulates contractile, ECM remodeling, ECM production or combinations thereof. In some instances, the lncRNA disclosed herein modulates ECM structure organization. In some instances, the lncRNA disclosed herein modulates extracellular matrix organization. In some instances, the lncRNA disclosed herein modulates extracellular structure organization. In some instances, the lncRNA disclosed herein modulates external encapsulating structure organization. In some instances, the lncRNA disclosed herein modulates the expression and/or components of ECM. Non-limiting examples of components of ECM include collagen, elastin, reticulin, associated-microfibrils, fibronectin, laminin, proteoglycan, proteoglycan polymer, glycoconjugate, glycosaminoglycan, or variants thereof. In some instances, the lncRNA disclosed herein modulates GTPase activity. In some instances, the lncRNA disclosed herein modulates activation of GTPase activity. Non-limiting examples of GTPase activity include signal transduction in response to activation of cell surface markers (e.g., transmembrane receptors), protein biosynthesis, regulation of cell differentiation, regulation of cell proliferation, regulation of cell division, regulation of cell movement, translocation of proteins, transportation of vesicles within cell or variations thereof. In some instances, the lncRNA disclosed herein modulates GTPase expression. In some instances, the lncRNA disclosed herein modulates GTP expression. In some instances, the lncRNA disclosed herein modulates the ability of GTPase to bind to GTP. In some instances, the lncRNA disclosed herein regulates genes involved in ECM structure organization, and/or GTPase activity. In some instances, the lncRNA disclosed herein induces or facilitates transition of fibroblast to CAFs. In some instances, the lncRNA disclosed herein induces or facilitates transition of fibroblast to myCAFs. In some instances, the lncRNA disclosed herein regulate metastasis formation and/or drug resistance. In some instances, the lncRNA disclosed herein increases expression of one or more markers of myCAFs in CAFs compared to control (e.g., cells with substantially no or low presence of lncRNA disclosed herein, or fibroblasts that are not associated with cancer). In some instances, the lncRNA disclosed herein increases expression of one or more genes in listed in Table 1 or Table 13 in CAFs compared to fibroblasts that are not associated with cancer. In some instances, the lncRNA disclosed herein increases expression of one or more genes listed in Table 1 or Table 13 in CAFs compared to CAFs without an activated TGFβ pathway. In some instances, the lncRNA disclosed herein increases expression of one or more genes listed in Table 1 or Table 13 in HDFs treated with TGFβ with or without starvation compared to HDFs without TGFβ treatment. In some instances, the lncRNA disclosed herein increases expression of one or more ECM-modulatory gene in CAFs when compared to a fibroblast that is not associated with cancer. ECM-modulatory genes comprise markers of myCAFs. Non-limiting examples of ECM-modulatory genes are LRRC15 (NM_001135057), MMP11 (NM 005940), COL11A1 (NM 080629), C1QTNF3 (NM 030945), CTHRC1 (NM_138455), COL12A1 (NM 004370), COL10A1 (NM 00493), COL5A2 (NM_000393), THBS2 (NM 003247), AEBP1 (NM 001129), ITGA11 (NM_001004439), PDPN (NM_006474), FAP (NM_004460), COL8A1 (NM 001850), COL1A1 (NM 000088), COL1A2 (NM_000089), FN1 (NM_212482), or POSTN (NM_006475). In some instances, higher expression of ECM-modulatory gene expression is associated with poor response to checkpoint blockade treatment, standard-of-care chemotherapy treatments of cancers, or a combination thereof. In some instances, higher expression of ECM-modulatory gene expression is associated with poor response to checkpoint blockade treatment of cancers.

### Modulators

In some aspects, the modulator disclosed herein is a modulator of lncRNAs (e.g., myCAF lncRNAs). In some instances, the modulator inhibits the transcription or decreases the amount of transcripts of the lncRNA. In some instances, the modulator upregulates the transcription or activity of the lncRNA. In some aspects, the modulator disclosed herein is a nucleic acid editing or modifying moiety. In some instances, the nucleic acid editing or modifying moiety targets the genomic region disclosed herein. In some instances, the nucleic acid editing or modifying moiety targets the long noncoding transcript. In some instances, the nucleic acid editing or modifying moiety targets a premature form of the long noncoding transcript.

In some aspects, the nucleic acid editing or modifying moiety is a programmable nucleic acid sequence specific endonuclease. In some instances, the nucleic acid editing or modifying moiety is a nucleic acid guided endonuclease. In some instances, the nucleic acid editing or modifying moiety is a CRISPR-based moiety. In other instances, the nucleic acid editing or modifying moiety is a meganuclease-based tool. In other instances, the nucleic acid editing or modifying moiety is a zinc finger nuclease (ZFN)-based moiety. In other aspects, the nucleic acid editing or modifying moiety is a transcription activator-like effector-based nuclease (TALEN)-based moiety. In other instances, the nucleic acid editing or modifying moiety is an Argonaute system.

In some aspects, the CRISPR-based moiety disclosed herein is a Type I, Type II, Type III, Type IV, Type V, or Type VI CRISPR system. CRISPR/Cas systems may be multi-protein systems or single effector protein systems. Multi-protein, or Class 1, CRISPR systems include Type I, Type III, and Type IV systems. In some instances, Class 2 systems include a single effector molecule and include Type II, Type V, and Type VI. In some aspects, the CRISPR-based moiety disclosed herein comprises a single or multiple effector proteins. An effector protein may comprise one or multiple nuclease domains. An effector protein may target DNA or RNA, and the DNA or RNA may be single stranded or double stranded. Effector proteins may generate double strand or single strand breaks. Effector proteins may comprise mutations in a nuclease domain thereby generating a nickase protein. Effector proteins may comprise mutations in one or more nuclease domains, thereby generating a catalytically dead nuclease that is able to bind but not cleave a target sequence.

In some aspects, the CRISPR-based moiety disclosed comprises a single or multiple guiding RNAs (gRNAs). In some instances, the gRNA disclosed herein targets a portion of chr3:194355288 to chr3:194370349 on the sense strand. In some instances, the gRNA disclosed herein targets a portion of chr3:194355288 to chr3:194370349 on the antisense strand. In some instances, the gRNA disclosed herein targets a portion upstream of chr3:194355288 to chr3:194370349. In some instances, the gRNA disclosed herein targets a portion downstream of chr3:194355288 to chr3:194370349. In some instances, the gRNA disclosed herein targets a portion of chr3:194355288 to chr3:194358966 on the sense strand. In some instances, the gRNA disclosed herein targets a portion of chr3:194355288 to chr3:194358966 on the antisense strand. In some instances, the gRNA disclosed herein targets a portion upstream of chr3:194355288 to chr3:194358966. In some instances, the gRNA disclosed herein targets a portion downstream of chr3:194355288 to chr3:194358966. In some instances, the gRNA disclosed herein targets a portion of chr3:194368496 to chr3:194370349 on the sense strand. In some instances, the gRNA disclosed herein targets a portion of chr3:194368496 to chr3:194370349 on the antisense strand. In some instances, the gRNA disclosed herein targets a portion upstream of chr3:194368496 to chr3:194370349. In some instances, the gRNA disclosed herein targets a portion downstream of chr3:194368496 to chr3:194370349. In some instances, the gRNA disclosed herein targets a portion of chr6:74958809 to chr6:75026433 on the sense strand. In some instances, the gRNA disclosed herein targets a portion of chr6:74958809 to chr6:75026433 on the antisense strand. In some instances, the gRNA disclosed herein targets a portion upstream of chr6:74958809 to chr6:75026433. In some instances, the gRNA disclosed herein targets a portion downstream of chr6:74958809 to chr6:75026433. In some instances, the gRNA disclosed herein targets a portion of chr1:66390975 to chr1:66516344 on the sense strand. In some instances, the gRNA disclosed herein targets a portion of chr1:66390975 to chr1:66516344 on the antisense strand. In some instances, the gRNA disclosed herein targets a portion upstream of chr1:66390975 to chr1:66516344. In some instances, the gRNA disclosed herein targets a portion downstream of chr1:66390975 to chr1:66516344. In some instances, the gRNA disclosed herein targets a portion of chr12:67394371 to chr12:67455635 on the sense strand. In some instances, the gRNA disclosed herein targets a portion of chr12:67394371 to chr12:67455635 on the antisense strand.

In some instances, the gRNA disclosed herein targets a portion of chr12:67394371 to chr12:67455635. In some instances, the gRNA disclosed herein targets a portion of chr12:67394371 to chr12:67590771 on the sense strand. In some instances, the gRNA disclosed herein targets a portion of chr12:67394371 to chr12:67590771 on the antisense strand. In some instances, the gRNA disclosed herein targets a portion upstream of chr12:67394371 to chr12:67590771. In some instances, the gRNA disclosed herein targets a portion downstream of chr12:67394371 to chr12:67590771. In some instances, the gRNA disclosed herein targets a portion of chr9:87219871 to chr9:87277312 on the sense strand. In some instances, the gRNA disclosed herein targets a portion of chr9:87219871 to chr9:87277312 on the antisense strand. In some instances, the gRNA disclosed herein targets a portion upstream of chr9:87219871 to chr9:87277312. In some instances, the gRNA disclosed herein targets a portion downstream of chr9:87219871 to chr9:87277312. In some instances, the gRNA disclosed herein targets a portion of chr2:215718043 to chr2:215720944 on the sense strand. In some instances, the gRNA disclosed herein targets a portion of chr2:215718043 to chr2:215720944 on the antisense strand. In some instances, the gRNA disclosed herein targets a portion upstream of chr2:215718043 to chr2:215720944. In some instances, the gRNA disclosed herein targets a portion downstream of chr2:215718043 to chr2:215720944. In some instances, the gRNA disclosed herein targets a portion of chr1:230710698 to chr1:230795492 on the sense strand. In some instances, the gRNA disclosed herein targets a portion of chr1:230710698 to chr1:230795492 on the antisense strand. In some instances, the gRNA disclosed herein targets a portion upstream of chr1:230710698 to chr1:230795492. In some instances, the gRNA disclosed herein targets a portion downstream of chr1:230710698 to chr1:230795492. The gRNA may comprise a crRNA. The gRNA may comprise a chimeric RNA with crRNA and tracrRNA sequences. The gRNA may comprise a separate crRNA and tracrRNA. Target nucleic acid sequences may comprise a protospacer adjacent motif (PAM) or a protospacer flanking site (PFS). The PAM or PFS may be 3′ or 5′ of the target or protospacer site. Cleavage of a target sequence may generate blunt ends, 3′ overhangs, or 5′ overhangs.

The gRNA disclosed herein may comprise a spacer sequence. Spacer sequences may be complementary to target sequences or protospacer sequences. Spacer sequences may be 10, 11, 12, 13, 14, 15, 16, 17, 18, 19, 20, 21, 22, 23, 24, 25, 26, 27, 28, 29, 30, 31, 32, 33, 34, 35, or 36 nucleotides in length. In some aspects, the spacer sequence may be less than 10 or more than 36 nucleotides in length.

The gRNA disclosed herein may comprise a repeat sequence. In some aspects, the repeat sequence is part of a double stranded portion of the gRNA. A repeat sequence may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some aspects, the spacer sequence may be less than 10 or more than 50 nucleotides in length.

The gRNA disclosed herein may comprise one or more synthetic nucleotides, non-naturally occurring nucleotides, nucleotides with a modification, deoxyribonucleotide, or any combination thereof. Additionally and/or alternatively, a gRNA may comprise a hairpin, linker region, single stranded region, double stranded region, or any combination thereof. Additionally or alternatively, a gRNA may comprise a signaling or reporter molecule.

The gRNA disclosed herein may be encoded by genetic or episomal DNA. The gRNA disclosed herein may be provided or delivered concomitantly with a CRISPR nuclease or sequentially. The gRNA disclosed herein may be chemically synthesized, in vitro transcribed or otherwise generated using standard RNA generation techniques known in the art.

The CRISPR-based moiety disclosed herein can be a Type II CRISPR system, for example a Cas9 system. The Type II nuclease can comprise a single effector protein, which, In some aspects, comprises a RuvC and HNH nuclease domains. In some aspects, a functional Type II nuclease may comprise two or more polypeptides, each of which comprises a nuclease domain or fragment thereof. The target nucleic acid sequences may comprise a 3′ protospacer adjacent motif (PAM). In some aspects, the PAM may be 5′ of the target nucleic acid. Guide RNAs (gRNA) may comprise a single chimeric gRNA, which contains both crRNA and tracrRNA sequences. In some instances, the gRNA may comprise a set of two RNAs, for example a crRNA and a tracrRNA. The Type II nuclease may generate a double strand break, which in some cases creates two blunt ends. In some aspects, the Type II CRISPR nuclease is engineered to be a nickase such that the nuclease only generates a single strand break. In such cases, two distinct nucleic acid sequences may be targeted by gRNAs such that two single strand breaks are generated by the nickase. In some aspects, the two single strand breaks effectively create a double strand break. In some aspects, where a Type II nickase is used to generate two single strand breaks, the resulting nucleic acid free ends may either be blunt, have a 3′ overhang, or a 5′ overhang. In some aspects, a Type II nuclease may be catalytically dead such that it binds to a target sequence, but does not cleave. For example, a Type II nuclease may have mutations in both the RuvC and HNH domains, thereby rendering both nuclease domains non-functional. A Type II CRISPR system may be one of three sub-types, namely Type II-A, Type II-B, or Type II-C.

The CRISPR-based moiety disclosed herein can be a Type V CRISPR system, for example a Cpf1, C2c1, or C2c3 system. The Type V nuclease may comprise a single effector protein, which comprises a single RuvC nuclease domain. In other cases, a function Type V nuclease comprises a RuvC domain split between two or more polypeptides. In such cases, the target nucleic acid sequences may comprise a 5′ PAM or 3′ PAM. Guide RNAs (gRNA) may comprise a single gRNA or single crRNA, such as may be the case with Cpf1. In some aspects, a tracrRNA is not needed. In other examples, such as when C2c1 is used, a gRNA may comprise a single chimeric gRNA, which contains both crRNA and tracrRNA sequences or the gRNA may comprise a set of two RNAs, for example a crRNA and a tracrRNA. The Type V CRISPR nuclease may generate a double strand break, which generates a 5′ overhang. In some aspects, the Type V CRISPR nuclease is engineered to be a nickase such that the nuclease only generates a single strand break. In such cases, two distinct nucleic acid sequences may be targeted by gRNAs such that two single strand breaks are generated by the nickase. In some aspects, the two single strand breaks effectively create a double strand break. In some aspects where a Type V nickase is used to generate two single strand breaks, the resulting nucleic acid free ends may either be blunt, have a 3′ overhang, or a 5′ overhang. In some aspects, a Type V nuclease may be catalytically dead such that it binds to a target sequence, but does not cleave. For example, a Type V nuclease may have mutations a RuvC domain, thereby rendering the nuclease domain non-functional.

The CRISPR-based moiety disclosed herein may be a Type VI CRISPR system, for example a C2c2 system. A Type VI nuclease may comprise a HEPN domain. In some aspects, the Type VI nuclease comprises two or more polypeptides, each of which comprises a HEPN nuclease domain or fragment thereof. In such cases, the target nucleic acid sequences may by RNA, such as single stranded RNA. When using Type VI CRISPR system, a target nucleic acid may comprise a protospacer flanking site (PFS). The PFS may be 3′ or 5′ or the target or protospacer sequence. Guide RNAs (gRNA) may comprise a single gRNA or single crRNA. In some aspects, a tracrRNA is not needed. In other examples, a gRNA may comprise a single chimeric gRNA, which contains both crRNA and tracrRNA sequences or the gRNA may comprise a set of two RNAs, for example a crRNA and a tracrRNA. In some aspects, a Type VI nuclease may be catalytically dead such that it binds to a target sequence, but does not cleave.

For example, a Type VI nuclease may have mutations in a HEPN domain, thereby rendering the nuclease domains non-functional.

Non-limiting examples of suitable nucleases, including nucleic acid-guided nucleases, for use in the present disclosure include C2c1, C2c2, C2c3, CasI, Cas1B, Cas2, Cas3, Cas4, Cas5, Cas6, Cas7, Cas8, Cas9 (also known as Csnl and Csxl2), CasIO, Cpf1, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, Csx100, Csx16, CsaX, Csx3, Csxl, Csx15, Csfl, Csf2, Csf3, Csf4, homologues thereof, orthologues thereof, or modified versions thereof.

In some aspects, The CRISPR-based moiety disclosed herein is an Argonaute (Ago) system. Ago protein may be derived from a prokaryote, eukaryote, or archaea. The target nucleic acid may be RNA or DNA. A DNA target may be single stranded or double stranded. In some aspects, the target nucleic acid does not require a specific target flanking sequence, such as a sequence equivalent to a protospacer adjacent motif or protospacer flanking sequence. The Ago protein may create a double strand break or single strand break. In some aspects, when an Ago protein forms a single strand break, two Ago proteins may be used in combination to generate a double strand break. In some aspects, an Ago protein comprises one, two, or more nuclease domains. In some aspects, an Ago protein comprises one, two, or more catalytic domains. One or more nuclease or catalytic domains may be mutated in the Ago protein, thereby generating a nickase protein capable of generating single strand breaks. In other aspects, mutations in one or more nuclease or catalytic domains of an Ago protein generates a catalytically dead Ago protein that may bind but not cleave a target nucleic acid.

Ago proteins may be targeted to target nucleic acid sequences by a guiding nucleic acid. In some aspects, the guiding nucleic acid is a guide DNA (gDNA). The gDNA may have a 5′ phosphorylated end. The gDNA may be single stranded or double stranded. Single stranded gDNA may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some aspects, the gDNA may be less than 10 nucleotides in length. In some aspects, the gDNA may be more than 50 nucleotides in length.

Argonaute-mediated cleavage may generate blunt end, 5′ overhangs, or 3′ overhangs. In some aspects, one or more nucleotides are removed from the target site during or following cleavage.

In some aspects, the nucleic acid editing or modifying moiety is a repressive dCas9 with the aid of a (single) guide RNA targeting the portion of the genomic region that is transcribed to the long noncoding transcript. In some instances, the nucleic acid editing or modifying moiety is dCas9-KRAB-MECP2 with the aid of a (single) guide RNA targeting the portion of the genomic region that is transcribed to the long noncoding transcript. In other specific aspects, the nucleic acid editing or modifying moiety is dCas9-KRAB-DNMT1 with the aid of a (single) guide RNA targeting the portion of the genomic region that is transcribed to the long noncoding transcript. In the above-mentioned aspects, the (single) guide RNA targets 5′ side of an enhancer region the genomic region that is transcribed to the long noncoding transcript. In the certain aspects, the (single) guide RNA targets 5′ side of an enhancer region the genomic region that is transcribed to the long noncoding transcript.

In some aspects, the modulator disclosed herein is a CRISPRi complex comprising a dCas9 and an sgRNA. In some aspects, the modulator disclosed herein is a dCas9 and an sgRNA targeting transcription starting site of the lncRNA disclosed herein, thereby the transcription of the lncRNA is inhibited. In some aspects, the sgRNA is designed using CRISPick (https://portals.broadinstitute.org/gppx/crispick/public) from the Broad institute. In some aspects, the sgRNA is designed to target −150 bp and +300 bp of the annotated transcriptional start site of the lncRNA disclosed herein. In some aspects, the sgRNA is designed to target −150 bp and +300 bp of the potential transcriptional start site of the lncRNA disclosed herein.

In some aspects, the modulator disclosed herein is a synthetic or an artificial oligonucleotide or a polynucleotide. In specific aspects, the synthetic or artificial oligonucleotide or polynucleotide is a small interfering RNA (siRNA), a microRNA (miRNA), an inhibitory double stranded RNA (dsRNA), a small or short hairpin RNA (shRNA), an antisense oligonucleotide (ASO), a piwi-interacting RNA (piRNA), a heterogeneous nuclear RNA (hnRNA), a small nuclear RNA (snRNA), an enzymatically-prepared siRNA (esiRNA), or the precursors thereof. In some instances, the nucleic acid molecule is an ASO. In some instances, the ASO is a gapmer or a mixmer. In some instances, the ASO is a modified oligonucleotide. In some instances, the ASO is a DNA analogue. In some instances, the ASO is modified with morpholino nucleotides. In some instances, the ASO is a phosphorodiamidate morpholino oligomer (PMO). In some instances, the nucleic acid molecule is single-stranded. In some instances, the nucleic acid is double-stranded.

In some instances, the ASO is about 6-50 nucleotides long. In some instances, the ASO is about 6-45, 6-40, 6-35, 6-30, 6-20, 6-18, 7-45, 7-40, 7-35, 7-30, 7-20, 7-18, 8-45, 8-40, 8-35, 8-30, 8-20, 8-18, 9-45, 9-40, 9-35, 9-30, 9-20, 9-18, 10-45, 10-40, 10-35, 10-20, 10-18, 11-30, 11-45, 11-40, 11-35, 11-30, 11-20, 11-18, 12-45, 12-40, 12-35, 12-30, 12-20, or 12-18 nucleotides long. In some instances, the ASO is about 12-30 nucleotides long. In some instances, the ASO is at least 6, 7, 8, 9, or 10 nucleotides long. In some instances, the ASO is at most 18, 20, 30, 35, 40, 45, 50, 55, or 60 nucleotides long.

In some instances, the modulator disclosed herein (e.g., ASO) targets the lncRNA transcribed from a genomic region or a subset thereof listed in Table 10 or Table 12. In some instances, the modulator disclosed herein (e.g., ASO) targets the lncRNA comprising a sequence or a fragment thereof listed in Table 11. In some instances, the modulator disclosed herein (e.g., ASO) targets the lncRNA sharing at least 80%, 85%, 90% or 95% sequence homology with a full-length sequence listed in Table 11. In some instances, the modulator disclosed herein (e.g., ASO) targets the lncRNA sharing at least 80%, 85%, 90% or 95% sequence homology with a fragment of a sequence listed in Table 11.

In some instances, the ASO comprises a sequence from SEQ ID NOs: 1-40. In some instances, the ASO comprises SEQ ID NO: 4. In some instances, the ASO comprises SEQ ID NO: 5. In some instances, the ASO comprises SEQ ID NO: 15. In some instances, the ASO comprises SEQ ID NO: 19. In some instances, the ASO comprises SEQ ID NO: 2. In some instances, the ASO comprises SEQ ID NO: 16. In some instances, the ASO comprises SEQ ID NO: 18. In some instances, the ASO comprises SEQ ID NO: 24. In some instances, the ASO comprises SEQ ID NO: 25. In some instances, the ASO comprises SEQ ID NO: 26. In some instances, the ASO comprises SEQ ID NO: 34. In some instances, the ASO comprises SEQ ID NO: 38. In some instances, the ASO comprises SEQ ID NO: 39. In some instances, the ASO comprises SEQ ID NO: 40. In some instances, the ASO comprises SEQ ID NO: 9. In some instances, the ASO comprises SEQ ID NO: 10. In some instances, the ASO comprises SEQ ID NO: 11. In some instances, the ASO comprises at least 9 consecutive nucleotides with no more than 1 mismatch from one of SEQ ID NOs: 1-40. In some instances, the ASO comprises at least 9 consecutive nucleotides with no more than 2 mismatches from one of SEQ ID NOs: 1-40. In some instances, the ASO comprises at least 9 consecutive nucleotides with no more than 3 mismatches from one of SEQ ID NOs: 1-40. In some instances, the ASO comprises at least 10 consecutive nucleotides with no more than 1 mismatch from one of SEQ ID NOs: 1-40. In some instances, the ASO comprises at least 10 consecutive nucleotides with no more than 2 mismatches from one of SEQ ID NOs: 1-40. In some instances, the ASO comprises at least 10 consecutive nucleotides with no more than 3 mismatches from one of SEQ ID NOs: 1-40. In some instances, the ASO comprises at least 11 consecutive nucleotides with no more than 1 mismatch from one of SEQ ID NOs: 1-40. In some instances, the ASO comprises at least 11 consecutive nucleotides with no more than 2 mismatches from one of SEQ ID NOs: 1-40. In some instances, the ASO comprises at least 11 consecutive nucleotides with no more than 3 mismatches from one of SEQ ID NOs: 1-40. In some instances, the ASO comprises at least 12 consecutive nucleotides with no more than 1 mismatch from one of SEQ ID NOs: 1-40. In some instances, the ASO comprises at least 12 consecutive nucleotides with no more than 2 mismatches from one of SEQ ID NOs: 1-40. In some instances, the ASO comprises at least 12 consecutive nucleotides with no more than 3 mismatches from one of SEQ ID NOs: 1-40. In some instances, the ASO comprises at least 13 consecutive nucleotides with no more than 1 mismatch from one of SEQ ID NOs: 1-40. In some instances, the ASO comprises at least 13 consecutive nucleotides with no more than 2 mismatches from one of SEQ ID NOs: 1-40. In some instances, the ASO comprises at least 13 consecutive nucleotides with no more than 3 mismatches from one of SEQ ID NOs: 1-40. In some instances, the ASO comprises a nucleic acid sequence of 80%, at least 85%, at least 90%, at least 95% identical to a sequence from SEQ ID NOs: 1-40. Table 2 shows sequences of ASO candidates targeting lncRNAs disclosed herein.

TABLE 2
Sequences of exemplary ASO candidates
targeting IncRNAs

| SEQ | | | |
|---|---|---|---|
| ID | ASO | | |
| NO: | name | ASO sequence | Target |
| 1 | SEAL1_ | GGGAATTGGGCTGTAT | human |
| | G1 | | XLOC_055514 |
| 2 | SEAL1_ | TGGTGTTCAATAGGCT | human |
| | G2 | | XLOC_055514 |
| 3 | SEAL1_ | GAAACCTCATCATCCG | human |
| | G3 | | XLOC_055514 |
| 4 | SEAL1_ | GAAGTTAGGCTCATGG | human |
| | G4 | | XLOC_055514 |
| 5 | SEAL1_ | GGTCTGGTTAGGAGTT | human |
| | G5 | | XLOC_055514 |
| 6 | SEAL1_ | TTATATCTGGAGACCC | human |
| | G6 | | XLOC_055514 |
| 7 | SEAL1_ | GATAGTGTATGGCTTG | human |
| | G7 | | XLOC_055514 |
| 8 | SEAL1_ | CAGTCACACGAATACC | human |
| | G8 | | XLOC_055514 |
| 9 | SEAL9_ | GATAACTCCTGCAAGT | human |
| | G1 | | XLOC_055515 |
| 10 | SEAL9_ | GATTGACCGTTCTGGA | human |
| | G2 | | XLOC_055515 |
| 11 | SEAL9_ | CTATGTGTACCCTGTA | human |
| | G3 | | XLOC_055515 |
| 12 | SEAL2_ | GTGCCTTGTGACTAGT | human |
| | G1 | | XLOC_069921 |
| 13 | SEAL2_ | CCCATTAGCCTATATG | human |
| | G2 | | XLOC_069921 |
| 14 | SEAL2_ | ATGTTAGTCCTCCTAG | human |
| | G3 | | XLOC_069921 |
| 15 | SEAL2_ | ATGTCCAATCCTACAC | human |
| | G4 | | XLOC_069921 |
| 16 | SEAL2_ | GTCTTTGACCAGTGTA | human |
| | G5 | | XLOC_069921 |
| 17 | SEAL2_ | GATCTAACTATCCACC | human |
| | G6 | | XLOC_069921 |
| 18 | SEAL2_ | CTGTGTGGAACTTATC | human |
| | G7 | | XLOC_069921 |
| 19 | SEAL2_ | ATGATATGCACCATCC | human |
| | G8 | | XLOC_069921 |
| 20 | SEAL3_ | GGTCTCACTAAAGGGT | human |
| | G1 | | XLOC_005184 |
| 21 | SEAL3_ | GCCCTATGTTAGTATC | human |
| | G2 | | XLOC_005184 |
| 22 | SEAL3_ | GTCAACCATTTGTAGG | human |
| | G3 | | XLOC_005184 |
| 23 | SEAL3_ | TATGTGGTAACAATCC | human |
| | G4 | | XLOC_005184 |
| 24 | SEAL3_ | GTGTCCCACCTATTAG | human |
| | G5 | | XLOC_005184 |
| 25 | SEAL3_ | CTTGCTCTAGTATGAC | human |
| | G6 | | XLOC_005184 |
| 26 | SEAL3_ | CCATGATAAGAGGGTC | human |
| | G7 | | XLOC_005184 |
| 27 | SEAL3_ | ACCTTATAGCACATGC | human |
| | G8 | | XLOC_005184 |
| 28 | SEAL4_ | GATTACCCACTTCAAG | human |
| | G1 | | ENSG00000203585/ |
| | | | SHARED_00113753 |
| 29 | SEAL4_ | GACACCTCAAGTTAGA | human |
| | G2 | | ENSG00000203585/ |
| | | | SHARED_00113753 |
| 30 | SEAL4_ | CAAGGGATGATAGCTC | human |
| | G3 | | ENSG00000203585/ |
| | | | SHARED_00113753 |
| 31 | SEAL4_ | AGGCATGGATTATACC | human |
| | G4 | | ENSG00000203585/ |
| | | | SHARED_00113753 |
| 32 | SEAL4_ | ACTAGGCTACATTACC | human |
| | G5 | | ENSG00000203585/ |
| | | | SHARED_00113753 |
| 33 | SEAL4_ | CCCTAGAATAAGTACC | human |
| | G6 | | ENSG00000203585/ |
| | | | SHARED_00113753 |
| 34 | SEAL4_ | GTCCCAATTTGAAGCC | human |
| | 753_G1 | | ENSG00000203585/ |
| | | | SHARED_00113753 |
| 35 | SEAL4_ | GGTAAGGAATACACTC | human |
| | 753_G2 | | ENSG00000203585/ |
| | | | SHARED_00113753 |
| 36 | SEAL4_ | TCCAAACTTAGTCAGC | human |
| | 753_G3 | | ENSG00000203585/ |
| | | | SHARED_00113753 |
| 37 | SEAL4_ | AACCATTAGAGTCCTC | human |
| | 753_G4 | | ENSG00000203585/ |
| | | | SHARED_00113753 |
| 38 | SEAL4_ | GCTGACCCATAATAGG | human |
| | 753_G5 | | ENSG00000203585/ |
| | | | SHARED_00113753 |
| 39 | SEAL4_ | GTAACCCATATCTGTG | human |
| | 753_G6 | | ENSG00000203585/ |
| | | | SHARED_00113753 |
| 40 | SEAL4_ | GTATCATTGTTCAGCC | human |
| | 753_G7 | | ENSG00000203585/ |
| | | | SHARED_00113753 |

Note:
ENSG00000203585 and SHARED_00113753 are two different transcripts transcribed from a genomic region within chr12: 67394371-67590771 (+).

In some instances, the ASO comprises any one of sequences set forth in Tables 3-9. In some instances, the ASO comprises at least 9 consecutive nucleotides with no more than 1 mismatch from sequences set forth in Tables 3-9. In some instances, the ASO comprises at least 9 consecutive nucleotides with no more than 2 mismatches from sequences set forth in Tables 3-9. In some instances, the ASO comprises at least 9 consecutive nucleotides with no more than 3 mismatches from sequences set forth in Tables 3-9. In some instances, the ASO comprises at least 10 consecutive nucleotides with no more than 1 mismatch from sequences set forth in Tables 3-9. In some instances, the ASO comprises at least 10 consecutive nucleotides with no more than 2 mismatches from sequences set forth in Tables 3-9. In some instances, the ASO comprises at least 10 consecutive nucleotides with no more than 3 mismatches from sequences set forth in Tables 3-9. In some instances, the ASO comprises at least 11 consecutive nucleotides with no more than 1 mismatch from sequences set forth in Tables 3-9. In some instances, the ASO comprises at least 11 consecutive nucleotides with no more than 2 mismatches from sequences set forth in Tables 3-9. In some instances, the ASO comprises at least 11 consecutive nucleotides with no more than 3 mismatches from sequences set forth in Tables 3-9. In some instances, the ASO comprises at least 12 consecutive nucleotides with no more than 1 mismatch from sequences set forth in Tables 3-9. In some instances, the ASO comprises at least 12 consecutive nucleotides with no more than 2 mismatches from sequences set forth in Tables 3-9. In some instances, the ASO comprises at least 12 consecutive nucleotides with no more than 3 mismatches from sequences set forth in Tables 3-9. In some instances, the ASO comprises at least 13 consecutive nucleotides with no more than 1 mismatch from sequences set forth in Tables 3-9. In some instances, the ASO comprises at least 13 consecutive nucleotides with no more than 2 mismatches from sequences set forth in Tables 3-9. In some instances, the ASO comprises at least 13 consecutive nucleotides with no more than 3 mismatches from sequences set forth in Tables 3-9. In some instances, the ASO comprises a nucleic acid sequence of 80%, at least 85%, at least 90%, at least 95% identical to sequences set forth in sequences set forth in Tables 3-9. Tables 3-9 show additional sequences of ASO candidates targeting lncRNAs disclosed herein and the criteria by which they were selected in silico. The ASO candidates were selected in silico based on the following criteria: 1) no off-target hybridization, 2) absence of questionable motifs, 3) target accessibility, 4) hybridization free energy, and 5) secondary structures.

TABLE 3
ASOs sequences targeting human
XLOC_055514 (Chr3)

| SEQ | |
|---|---|
| ID | |
| NO. | Sequence |
| 41 | TGTAGAGTGTGAGCCC |
| 42 | TTGTAGAGTGTGAGCC |
| 43 | GCTAGATCATGTTTGC |
| 44 | TAGATGTATCCTAGCT |
| 45 | TTAGATGTATCCTAGC |
| 46 | GTTAGATGTATCCTAG |
| 47 | GATGTTAGATGTATCC |
| 48 | GGGAATTGGGCTGTAT |
| 49 | GAATAGTTTGTCCCTC |
| 50 | TGGTGTTCAATAGGCT |
| 51 | ATGGTGTTCAATAGGC |
| 52 | GAAACCTCATCATCCG |
| 53 | ATCACCCGCTGATGGT |
| 54 | ATGAGCATCACCCGCT |
| 55 | ATCGGCTCTTATTAGC |
| 56 | GATCGGCTCTTATTAG |
| 57 | ACCACCAATCCCGATC |
| 58 | TACCACCAATCCCGAT |
| 59 | CTACCACCAATCCCGA |
| 60 | CCGCTCCACAAGACAC |
| 61 | ATCGGCTCTTATTAGC |
| 62 | GATCGGCTCTTATTAG |
| 63 | ACCACCAATCCCGATC |
| 64 | CCGCTCCACAAGACAC |
| 65 | ATCGGCTCTTATTAGC |
| 66 | GATCGGCTCTTATTAG |
| 67 | GAAGTTAGGCTCATGG |
| 68 | GTCACACGAATACCTG |
| 69 | AGTCACACGAATACCT |
| 70 | CAGTCACACGAATACC |
| 71 | GGTCTGGTTAGGAGTT |
| 72 | TGGTCTGGTTAGGAGT |
| 73 | TTATATCTGGAGACCC |
| 74 | GATAGTGTATGGCTTG |
| 75 | GGATGCTGCTTGTAATC |
| 76 | GATCATGTTTGCCTTGC |
| 77 | GATTGTTCTCACCAACC |
| 78 | TGTTAGATGTATCCTAG |
| 79 | TAGAATAGTTTGTCCCT |
| 80 | AGAAGCATCCTGACTGC |
| 81 | TTGTAGAGTGTGAGCCCT |
| 82 | AGATCATGTTTGCCTTGC |
| 83 | GACAGCTAGAGATAACTC |
| 84 | GAGATACAAGAGTTCTAC |
| 85 | TCATCAGAACCTCACTTG |
| 86 | ACTCTTCAAGCACTGGAC |
| 87 | TTGACAGTCCCACGCCCA |
| 88 | GCCCAGGAAAGAACTTCA |
| 89 | GACGTGATATGCCTGTAT |
| 90 | CATGATAGTGTATGGCTT |
| 91 | GATGCTGCTTGTAATCCAT |
| 92 | CTGGATGCTGCTTGTAATC |
| 93 | GGGTCTGTCTTCTGCTGGA |
| 94 | TTTGTAGAGTGTGAGCCCT |
| 95 | GAATTACTGCAAATCAGCC |
| 96 | GATTGTTCTCACCAACCCG |
| 97 | ACTGAATCTGCGTTGTTGG |
| 98 | GAACTGAATCTGCGTTGTT |
| 99 | TCATGGGAATTGGGCTGTA |
| 100 | GCACAGATACTCTTCAAGC |
| 101 | TCTTAACCCAGAACCCTTA |
| 102 | TGGAAATCTACCACCAATC |
| 103 | GACGTGATATGCCTGTATC |
| 104 | TGATAGTGTATGGCTTGGT |
| 105 | GATGCTGCTTGTAATCCATT |
| 106 | TGTAGAGTGTGAGCCCTGGT |
| 107 | ATGTTTGCCTTGCTTAGAGA |
| 108 | ATCTGCGTTGTTGGGAAGCC |
| 109 | GATACAAGAGTTCTACTTAG |
| 110 | TGCACAGATACTCTTCAAGC |
| 111 | CCTTCCAGTTCCTGCACAAA |

TABLE 4
ASO sequences targeting human
XLOC_069921 (Chr6)

| SEQ | |
|---|---|
| ID | |
| NO. | Sequence |
| 112 | GATGTGCTTATACTGC |
| 113 | GGCACTCAACTCTATG |
| 114 | ACCAGTCTATTGTAAC |
| 115 | GCATAACCAGTCTATT |
| 116 | GGCATAACCAGTCTAT |
| 117 | CTTAATTGCCAACTAG |
| 118 | GTGCCTTGTGACTAGT |
| 119 | GATTAACACCAATTGC |
| 120 | ACCCTATGCTACTGTA |
| 121 | TAACCCTATGCTACTG |
| 122 | GTTTAACCCTATGCTA |
| 123 | AGTTTAACCCTATGCT |
| 124 | CAGTTTAACCCTATGC |
| 125 | ACAGTTTAACCCTATG |
| 126 | CCCATTAGCCTATATG |
| 127 | GCACCAGTATGACATT |
| 128 | ATGTTGAGCCATGCCC |
| 129 | GATGTTGAGCCATGCC |
| 130 | CACCAGATGTTGAGCC |
| 131 | GGCTTGATGTGTCCTC |
| 132 | GTGGCTTGATGTGTCC |
| 133 | GAGCCTAATGTATGCC |
| 134 | GTTACTAAGCCAATCT |
| 135 | TAGTCCTCCTAGCAGC |
| 136 | ATGTTAGTCCTCCTAG |
| 137 | GTACCATATGTTAGTC |
| 138 | GAGTACCATATGTTAG |
| 139 | AACAGTCAGAGTATCC |
| 140 | TAACTATGAGGTCACC |
| 141 | TATAATGCTAGATCCC |
| 142 | GTATAATGCTAGATCC |
| 143 | TCATTGTTAGGACCAC |
| 144 | AGGTCACTCTAGTAAC |
| 145 | TAGGTCACTCTAGTAA |
| 146 | ATAGGTCACTCTAGTA |
| 147 | AATAGGTCACTCTAGT |
| 148 | GGGAAATAGGTCACTC |
| 149 | ATGTCCAATCCTACAC |
| 150 | GCCATGTCCAATCCTA |
| 151 | GGTTTAGTAGGGATAT |
| 152 | TTAGGGTTTAGTAGGG |
| 153 | GTCTTTGACCAGTGTA |
| 154 | GGGTTGCATGAGGTGT |
| 155 | CAATTCACTGGTTGGA |
| 156 | GATCTAACTATCCACC |
| 157 | TAGACTGCAACTAAGT |
| 158 | GTGTGGCTCATTAGGC |
| 159 | TACATCTCATAGTACC |
| 160 | CTACATCTCATAGTAC |
| 161 | CCTACATCTCATAGTA |
| 162 | GTGTGGAACTTATCAA |
| 163 | CTGTGTGGAACTTATC |
| 164 | GATATGCACCATCCAT |
| 165 | ATGATATGCACCATCC |
| 166 | GCCAGCCTTAGTTTAA |
| 167 | ATTAGAGTTGGCATAC |
| 168 | GTCTAAGTTGCAGGAG |
| 169 | TAGTCTAAGTTGCAGG |
| 170 | ATGAGTACCAGCAACC |
| 171 | GATGGCAATGAGTACC |
| 172 | ATAGGACATCAGTTGC |
| 173 | GCATCAATTAGATCTC |
| 174 | GGCATCAATTAGATCT |
| 175 | ACCAAATGATGTGCTTA |
| 176 | CACCAAATGATGTGCTT |
| 177 | ACACCAAATGATGTGCT |
| 178 | GACACCAAATGATGTGC |
| 179 | TGACACCAAATGATGTG |
| 180 | ACTCAACTCTATGACAC |
| 181 | CACTCAACTCTATGACA |
| 182 | GCACTCAACTCTATGAC |
| 183 | GGCACTCAACTCTATGA |
| 184 | CAGAAGCAACTAATTTC |
| 185 | CCAGAAGCAACTAATTT |
| 186 | CCCAGAAGCAACTAATT |
| 187 | GCCTCACTGTTACTAAA |
| 188 | CTTGTGGTGGGATGGTG |
| 189 | TCTTGTGGTGGGATGGT |
| 190 | TTCTTGTGGTGGGATGG |
| 191 | TACTCACACTATATTGC |
| 192 | GACCTTAGAGAATACTC |
| 193 | TGACCTTAGAGAATACT |
| 194 | TTGACCTTAGAGAATAC |
| 195 | AGCATATCTTACTGTTC |
| 196 | CCACTTTAAGTAAGCAT |
| 197 | TCCACTTTAAGTAAGCA |
| 198 | GCCTATATGATTATCAG |
| 199 | CCATTAGCCTATATGAT |
| 200 | CCCATTAGCCTATATGA |
| 201 | TCCCATTAGCCTATATG |
| 202 | CTCACCACAGTATAGTA |
| 203 | TCTCACCACAGTATAGT |
| 204 | ATCTCACCACAGTATAG |
| 205 | CATCTCACCACAGTATA |
| 206 | TCATCTCACCACAGTAT |
| 207 | GTCATCTCACCACAGTA |
| 208 | TAGAGAAGCACCAAACT |
| 209 | TTAGAGAAGCACCAAAC |
| 210 | GGGTGTTGTAATTTGTA |
| 211 | CAGGAACTTAACAAAGT |
| 212 | ACCAGATGTTGAGCCAT |
| 213 | CACCAGATGTTGAGCCA |
| 214 | GTTAGGACCACAGATAG |
| 215 | GCATATCTGTACCATAC |
| 216 | AAACTTGTTCTGTGCCA |
| 217 | AGAACAATTCACTGGTT |
| 218 | CATCAGATCATGGCAAA |
| 219 | CCCTACATCTCATAGTA |
| 220 | GTCAGCATCTAGCAAAT |
| 221 | CTATTAGTCTAAGTTGC |
| 222 | CACTAACAAGGCTGTTC |
| 223 | GACACCAAATGATGTGCT |
| 224 | TGGCACTCAACTCTATGA |
| 225 | CATCCATAGGAGATTGAT |
| 226 | TGTATAATGCTAGATCCC |
| 227 | TGTGTGAACAATGTGGTG |
| 228 | CAAACCTTACCTATAGTG |
| 229 | GTGAACTTTAGACCAATG |
| 230 | ATCTCGATCTCTATAGCA |
| 231 | AACACGGCTTCTCACCAG |
| 232 | CACTAACAAGGCTGTTCA |
| 233 | ATGACACCAAATGATGTGC |
| 234 | CCAGAAGCAACTAATTTCA |
| 235 | GCATAACCAGTCTATTGTA |
| 236 | CTACAGAAGGATGCAAATC |
| 237 | CACTTAATTGCCAACTAGA |
| 238 | CATTCTATTTGCTGTGACA |
| 239 | GGTTGTTTGTGCTGGTCTC |
| 240 | ACAGTTTAACCCTATGCTA |
| 241 | TGATCTGCAACCACAAGTA |
| 242 | GTAGTAGGTGTAGATATGC |
| 243 | CAGGCATCAATTAGATCTC |
| 244 | CACTTAATTGCCAACTAGAA |
| 245 | GTTTAACCCTATGCTACTGT |
| 246 | ATTGCATATCTGTACCATAC |
| 247 | AGTGCTACAAACTATTCAGT |
| 248 | TGCAAGCATTTATTCTAGTG |
| 249 | CCTGACCTACATTTGCAACT |
| 250 | GCCTGACCTACATTTGCAAC |
| 251 | TGGTATAACAAATTAGGGCC |
| 252 | ATGGTATAACAAATTAGGGC |
| 253 | GATATGCACCATCCATCAAT |
| 254 | TGATATGCACCATCCATCAA |
| 255 | ATTGCCAGCCTTAGTTTAAA |
| 256 | CATTGCCAGCCTTAGTTTAA |
| 257 | TCATTGCCAGCCTTAGTTTA |
| 258 | AACTGCTGGAAACCTCACTT |
| 259 | GAACTGCTGGAAACCTCACT |
| 260 | TTAGTCTAAGTTGCAGGAGT |
| 261 | ATTAGTCTAAGTTGCAGGAG |
| 262 | TATTAGTCTAAGTTGCAGGA |
| 263 | CTATTAGTCTAAGTTGCAGG |
| 264 | TTGATGGCAATGAGTACCAG |
| 265 | GTTGATGGCAATGAGTACCA |
| 266 | AGTTGATGGCAATGAGTACC |
| 267 | AAGTTGATGGCAATGAGTAC |
| 268 | ACATCAGTTGCAGAAGGACT |
| 269 | CCAGGCATCAATTAGATCTC |
| 270 | CCACTAACAAGGCTGTTCAG |
| 271 | ACCACTAACAAGGCTGTTCA |
| 272 | AACCACTAACAAGGCTGTTC |
| 273 | AAACCACTAACAAGGCTGTT |
| 274 | TAAACCACTAACAAGGCTGT |

TABLE 5
ASO sequences targeting human
XLOC_005184 (Chr1)

| SEQ | |
|---|---|
| ID | |
| NO. | Sequence |
| 275 | GGTCTCACTAAAGGGT |
| 276 | CGATCTTCAAAGGGAC |
| 277 | GCGATCTTCAAAGGGA |
| 278 | AGGCGATCTTCAAAGG |
| 279 | TAGTGAGGCGATCTTC |
| 280 | CTAGTGAGGCGATCTT |
| 281 | TCTAGTGAGGCGATCT |
| 282 | ATCAACTAAAGCACCC |
| 283 | GCCCTATGTTAGTATC |
| 284 | TGCCCTATGTTAGTAT |
| 285 | TTGCCCTATGTTAGTA |
| 286 | GTTTGCCCTATGTTAG |
| 287 | CTTTAAGGCGTGATTT |
| 288 | TTCTTACAGAGCAACG |
| 289 | GCAGGATGAGTAGTTC |
| 290 | ACAATAGGTGGTATAC |
| 291 | GTTACAATAGGTGGTA |
| 292 | GGTTACAATAGGTGGT |
| 293 | AGCTCTGTATCGCAAA |
| 294 | AAGCTCTGTATCGCAA |
| 295 | CAAGCTCTGTATCGCA |
| 296 | TCAAGCTCTGTATCGC |
| 297 | ATCAAGCTCTGTATCG |
| 298 | GTAATGAGAGTTATCC |
| 299 | ATGATCCTCCTATGAC |
| 300 | GGCTATGATCCTCCTA |
| 301 | TTGTAGGAGCAGCATA |
| 302 | GTCAACCATTTGTAGG |
| 303 | CAATCCTACCTATTGA |
| 304 | TATGTGGTAACAATCC |
| 305 | GTATGTGGTAACAATC |
| 306 | GTGTATTGGGAGTATG |
| 307 | GTCCATCCAAGTCATG |
| 308 | AATCTTGAGCTACTAC |
| 309 | GTGAGGTTCCGATAAA |
| 310 | GGTGAGGTTCCGATAA |
| 311 | TGGTGAGGTTCCGATA |
| 312 | GGTTGGTGAGGTTCCG |
| 313 | GGGTTGGTGAGGTTCC |
| 314 | CACCCGAAGTTTGCAA |
| 315 | TCACCCGAAGTTTGCA |
| 316 | TTCACCCGAAGTTTGC |
| 317 | TTTCACCCGAAGTTTG |
| 318 | TACATTTCACCCGAAG |
| 319 | GCTAATACTAATGTCC |
| 320 | GGCTAATACTAATGTC |
| 321 | CATAGGTGGTTTCCGG |
| 322 | TCATAGGTGGTTTCCG |
| 323 | ACCTCAGTACCTGCGT |
| 324 | AACCTCAGTACCTGCG |
| 325 | GTGTCCCACCTATTAG |
| 326 | ATAGTAGGTTCTAGGG |
| 327 | GATAGTAGGTTCTAGG |
| 328 | GGATAGTAGGTTCTAG |
| 329 | TGGATAGTAGGTTCTA |
| 330 | GTGGATAGTAGGTTCT |
| 331 | AGTGGATAGTAGGTTC |
| 332 | TAGTGGATAGTAGGTT |
| 333 | GCATGGTATAGTGGAT |
| 334 | CGAACACACTCTTCTA |
| 335 | GCCAAACCCGCACATG |
| 336 | ATGCCAAACCCGCACA |
| 337 | TATGCCAAACCCGCAC |
| 338 | CATATGCCAAACCCGC |
| 339 | CCATATGCCAAACCCG |
| 340 | ACGTCTATCAATGGTA |
| 341 | GCTAGGGCTGATTCAT |
| 342 | GACTATGAGTAATTCC |
| 343 | CACGATACATAGCAAT |
| 344 | GTCAGTTAGAAGTCAC |
| 345 | GTATGACCTGGACCCA |
| 346 | AGTATGACCTGGACCC |
| 347 | TAGTATGACCTGGACC |
| 348 | CTCTAGTATGACCTGG |
| 349 | GCTCTAGTATGACCTG |
| 350 | TTGCTCTAGTATGACC |
| 351 | CTTGCTCTAGTATGAC |
| 352 | CGTGACCACCAGAGAT |
| 353 | CCGTGACCACCAGAGA |
| 354 | ACCGTGACCACCAGAG |
| 355 | AACCGTGACCACCAGA |
| 356 | CAACCGTGACCACCAG |
| 357 | AACAACCGTGACCACC |
| 358 | CAACAACCGTGACCAC |
| 359 | CCAACAACCGTGACCA |
| 360 | CCCAACAACCGTGACC |
| 361 | TCCCAACAACCGTGAC |
| 362 | GACTCCCAACAACCGT |
| 363 | TGACTCCCAACAACCG |
| 364 | GCCAACTGCCACTAGT |
| 365 | GTGGACTAAGATTGCT |
| 366 | TGTGGACTAAGATTGC |
| 367 | GTTATCTGAGGGAATC |
| 368 | GTACTTGTGTTGGTCA |
| 369 | AGTGTACTTGTGTTGG |
| 370 | CGACCTCACAGCAAGG |
| 371 | CCGACCTCACAGCAAG |
| 372 | TACACCGACCTCACAG |
| 373 | ATACACCGACCTCACA |
| 374 | AATACACCGACCTCAC |
| 375 | TCAATACACCGACCTC |
| 376 | ATCAATACACCGACCT |
| 377 | GATCAATACACCGACC |
| 378 | GTATGAAGTCTGCCCA |
| 379 | AGTATGAAGTCTGCCC |
| 380 | TGTCTTTCGGAGCTAC |
| 381 | CTGTCTTTCGGAGCTA |
| 382 | ACTAGGAGGTAATCTG |
| 383 | TAACTAGGAGGTAATC |
| 384 | CGTTACCTCCTAGTAA |
| 385 | GTCCGTCTATAATCAT |
| 386 | TATGAGTCGGGAATCA |
| 387 | GTATGAGTCGGGAATC |
| 388 | TGTATGAGTCGGGAAT |
| 389 | AATCTGTATGAGTCGG |
| 390 | TGCTAGGTGAGTATCA |
| 391 | CTGCTAGGTGAGTATC |
| 392 | GGTCCCAGGTAAGTTA |
| 393 | GGTTCCTGTAGTTGAT |
| 394 | GACTGGAATATAGCCG |
| 395 | TGCTATTTGGACTGTC |
| 396 | ACCCTATTGTCATGGT |
| 397 | TACCCTATTGTCATGG |
| 398 | CATGATAAGAGGGTCC |
| 399 | CCATGATAAGAGGGTC |
| 400 | GTACTGGAATGAGTCT |
| 401 | CGTTCTGATTGGTGTT |
| 402 | CAATAGCCGTTCTGAT |
| 403 | TCAATAGCCGTTCTGA |
| 404 | ATCAATAGCCGTTCTG |
| 405 | TATCAATAGCCGTTCT |
| 406 | ATATCAATAGCCGTTC |
| 407 | CATATCAATAGCCGTT |
| 408 | CCATATCAATAGCCGT |
| 409 | ACCATATCAATAGCCG |
| 410 | CTTGAGGTATTAGACC |
| 411 | ACCGAACCACTCCATC |
| 412 | AACCGAACCACTCCAT |
| 413 | CAACCGAACCACTCCA |
| 414 | GCAACCGAACCACTCC |
| 415 | TGCAACCGAACCACTC |
| 416 | TATCTTGCGGTGTGTG |
| 417 | GTATCTTGCGGTGTGT |
| 418 | TGTATCTTGCGGTGTG |
| 419 | GTGCTCCAATGATATC |
| 420 | TTGCGGTCACACATTT |
| 421 | ATTGCGGTCACACATT |
| 422 | TATTGCGGTCACACAT |
| 423 | CTATTGCGGTCACACA |
| 424 | ACCTTATAGCACATGC |
| 425 | TATCACACTACTTCGA |
| 426 | TAATAAAGCACGGTCT |
| 427 | TTAATAAAGCACGGTC |
| 428 | CATTAATAAAGCACGG |
| 429 | GGTTCATACGCATTTA |
| 430 | AGGTTCATACGCATTT |
| 431 | TAGGTTCATACGCATT |
| 432 | TTAGGTTCATACGCAT |
| 433 | GTTAGGTTCATACGCA |
| 434 | TGTTAGGTTCATACGC |
| 435 | TTGTTAGGTTCATACG |
| 436 | GGCAATCTTGGTGTAC |
| 437 | TGGCAATCTTGGTGTA |
| 438 | TCAGTACGATCAGAGA |
| 439 | TTCAGTACGATCAGAG |
| 440 | TTTCAGTACGATCAGA |
| 441 | ATTTCAGTACGATCAG |
| 442 | CAATTTCAGTACGATC |
| 443 | GATCAATTTCAGTACG |
| 444 | AAGAAACTCACAATGCC |
| 445 | GAAGAAACTCACAATGC |
| 446 | TGCGTGGAGACCAGATG |
| 447 | CCATATGCAAACTATAG |
| 448 | CACCATATGCAAACTAT |
| 449 | AAACACTGTTCTATCCC |
| 450 | TAAACACTGTTCTATCC |
| 451 | GTAAACACTGTTCTATC |
| 452 | CAAGTAAACACTGTTCT |
| 453 | CCAAGTAAACACTGTTC |
| 454 | GCCAAGTAAACACTGTT |
| 455 | AGCCAAGTAAACACTGT |
| 456 | CAGCCAAGTAAACACTG |
| 457 | ACAGCCAAGTAAACACT |
| 458 | CACAGCCAAGTAAACAC |
| 459 | GCATAAAGCTACACAGC |
| 460 | TTGGGACACAAGGCATA |
| 461 | GCCAATTTAACCATCCA |
| 462 | ATGCCAATTTAACCATC |
| 463 | GATGCCAATTTAACCAT |
| 464 | CGATGCCAATTTAACCA |
| 465 | TCGATGCCAATTTAACC |
| 466 | AGTCGATGCCAATTTAA |
| 467 | AAGTCGATGCCAATTTA |
| 468 | AAAGTCGATGCCAATTT |
| 469 | TAAAGTCGATGCCAATT |
| 470 | TTAAAGTCGATGCCAAT |
| 471 | GTTAAAGTCGATGCCAA |
| 472 | CCTTGATGATCTGTTAG |
| 473 | GCCTTGATGATCTGTTA |
| 474 | AGCCTTGATGATCTGTT |
| 475 | AAGTCAGCCTTGATGAT |
| 476 | AAAGTCAGCCTTGATGA |
| 477 | GAAAGTCAGCCTTGATG |
| 478 | AGAAAGTCAGCCTTGAT |
| 479 | GAGAAAGTCAGCCTTGA |
| 480 | AGAGAAAGTCAGCCTTG |
| 481 | CAAGAGAAAGTCAGCCT |
| 482 | CCAAGAGAAAGTCAGCC |
| 483 | CCCAAGAGAAAGTCAGC |
| 484 | CTGCAAGTGAGAGATGC |
| 485 | TCTGCAAGTGAGAGATG |
| 486 | GTGTTCTCTGCAAGTGA |
| 487 | TGTGTTCTCTGCAAGTG |
| 488 | ATGTGTTCTCTGCAAGT |
| 489 | ACATTGCTATTAGAGTC |
| 490 | GACATTGCTATTAGAGT |
| 491 | TGACATTGCTATTAGAG |
| 492 | GTGACATTGCTATTAGA |
| 493 | TGTGACATTGCTATTAG |
| 494 | GTAAATACTCCAGGTTT |
| 495 | TGTAAATACTCCAGGTT |
| 496 | ATGTAAATACTCCAGGT |
| 497 | AATGTAAATACTCCAGG |
| 498 | TCAGTGGCAGATATTAC |
| 499 | TTCAGTGGCAGATATTA |
| 500 | GTTCAGTGGCAGATATT |
| 501 | AGTTCAGTGGCAGATAT |
| 502 | CAGTTCAGTGGCAGATA |
| 503 | GCAGTTCAGTGGCAGAT |
| 504 | ATTTGCAGTTCAGTGGC |
| 505 | AATTTGCAGTTCAGTGG |
| 506 | CAATTTGCAGTTCAGTG |
| 507 | ACAATTTGCAGTTCAGT |
| 508 | AACAATTTGCAGTTCAG |
| 509 | GGTGAGGTTCCGATAAA |
| 510 | CCCAAGTTTGCCTGTGA |
| 511 | ACTAACACTGCCATCCC |
| 512 | TACTAACACTGCCATCC |
| 513 | ATACTAACACTGCCATC |
| 514 | AATACTAACACTGCCAT |
| 515 | GAATACTAACACTGCCA |
| 516 | AGAATACTAACACTGCC |
| 517 | GAAACTTGAATTGGTTC |
| 518 | GGAAACTTGAATTGGTT |
| 519 | GGGAAACTTGAATTGGT |
| 520 | TACAAAGATTGGGCATG |
| 521 | TTACAAAGATTGGGCAT |
| 522 | CTTACAAAGATTGGGCA |
| 523 | TCTTACAAAGATTGGGC |
| 524 | CTCTTACAAAGATTGGG |
| 525 | ACTCTTACAAAGATTGG |
| 526 | ATGGAAATGACTGTGTA |
| 527 | CTATGGAAATGACTGTG |
| 528 | ACCGAACCACTCCATCT |
| 529 | AACCGAACCACTCCATC |
| 530 | CAACCGAACCACTCCAT |
| 531 | GCAACCGAACCACTCCA |
| 532 | TGCAACCGAACCACTCC |
| 533 | CTGCAACCGAACCACTC |
| 534 | CCTGCAACCGAACCACT |
| 535 | CCCTGCAACCGAACCAC |
| 536 | CATTGTCTGCTCCAGGA |
| 537 | CCATTGTCTGCTCCAGG |
| 538 | GTAATCCCATTGTCTGC |
| 539 | AGTAATCCCATTGTCTG |
| 540 | CAGTAATCCCATTGTCT |
| 541 | CCAGTAATCCCATTGTC |
| 542 | TCAGCCTTTACTCCAAA |
| | G |
| 543 | ATCAGCCTTTACTCCAA |
| | A |
| 544 | GATCAGCCTTTACTCCA |
| | A |
| 545 | GGGCACTCTCACATTGG |
| | G |
| 546 | AGGGCACTCTCACATTG |
| | G |
| 547 | CTGCAAGTGAGAGATGC |
| | C |
| 548 | TCTGCAAGTGAGAGATG |
| | C |
| 549 | TGTGTTCTCTGCAAGTG |
| | A |
| 550 | ATGTGTTCTCTGCAAGT |
| | G |
| 551 | TATGTGTTCTCTGCAAGT |
| 552 | GTATGTGTTCTCTGCAA |
| | G |
| 553 | TGTATGTGTTCTCTGCAA |
| 554 | ATGTATGTGTTCTCTGCA |
| 555 | AATGTATGTGTTCTCTGC |
| 556 | GCGATCTTCAAAGGGAC |
| | C |
| 557 | TCTAGTGAGGCGATCTTC |
| 558 | TCAGCCAGATGAAAGATT |
| 559 | ATCCTTCAGCCAGATGAA |
| 560 | AGTCCAAGTCTCTTAGAT |
| 561 | TAGTCCAAGTCTCTTAGA |
| 562 | CTAGTCCAAGTCTCTTAG |
| 563 | TGATCCTGAGAAACTAGT |
| 564 | ATGATCCTGAGAAACTAG |
| 565 | TGCCCAAATCTATATGCT |
| 566 | GTGCCCAAATCTATATGC |
| 567 | AGTGCCCAAATCTATATG |
| 568 | GAGTGCCCAAATCTATAT |
| 569 | AGAGTGCCCAAATCTATA |
| 570 | CAGAGTGCCCAAATCTAT |
| 571 | ACAGAGTGCCCAAATCTA |
| 572 | GACAGAGTGCCCAAATCT |
| 573 | CTTAATCACAAGCACACA |
| 574 | ACTTAATCACAAGCACAC |
| 575 | GAACTTAATCACAAGCAC |
| 576 | CAGAACTTAATCACAAGC |
| 577 | GGTCAGAACTTAATCACA |
| 578 | TGGTCAGAACTTAATCAC |
| 579 | CTTGGTCAGAACTTAATC |
| 580 | CCTTGGTCAGAACTTAAT |
| 581 | GCTTCCAAGTTCATCAGC |
| 582 | GGCTTCCAAGTTCATCAG |
| 583 | AGGCTTCCAAGTTCATCA |
| 584 | AAGGCTTCCAAGTTCATC |
| 585 | AAAGGCTTCCAAGTTCAT |
| 586 | CAAAGGCTTCCAAGTTCA |
| 587 | TCAAAGGCTTCCAAGTTC |
| 588 | TTCAAAGGCTTCCAAGTT |
| 589 | GCACATAGCTTCAAAGGC |
| 590 | GGCACATAGCTTCAAAGG |
| 591 | ACATATTGATTCTGGGCC |
| 592 | CACATATTGATTCTGGGC |
| 593 | TCCACATATTGATTCTGG |
| 594 | AATTAATGACAGGCTCCC |
| 595 | TAATTAATGACAGGCTCC |
| 596 | CTAATTAATGACAGGCTC |
| 597 | CCTAATTAATGACAGGCT |
| 598 | TGCCTAATTAATGACAGG |
| 599 | GACAGTTCTTACATTAGC |
| 600 | CGTCTAAAGCAGCCTTGC |
| 601 | CCGTCTAAAGCAGCCTTG |
| 602 | TCCGTCTAAAGCAGCCTT |
| 603 | TTCCGTCTAAAGCAGCCT |
| 604 | ATTCCGTCTAAAGCAGCC |
| 605 | AATTCCGTCTAAAGCAGC |
| 606 | GTATTGGGAGTATGTGGT |
| 607 | TGTATTGGGAGTATGTGG |
| 608 | GTGTATTGGGAGTATGTG |
| 609 | AGTGTATTGGGAGTATGT |
| 610 | CTGTGAAAGTGTATTGGG |
| 611 | TCTGTGAAAGTGTATTGG |
| 612 | ATGGTGAAATGGGTATTC |
| 613 | CATGGTGAAATGGGTATT |
| 614 | TGTTAATGAAGGCATGGT |
| 615 | GGAAACCCTATTCTTTGA |
| 616 | GCATGACAACTATTTCCA |
| 617 | AGCATGACAACTATTTCC |
| 618 | AGTTTGCCTGTGAAATTC |
| 619 | CAAGTTTGCCTGTGAAAT |
| 620 | CCAAGTTTGCCTGTGAAA |
| 621 | CTTCCAAAGACTTCGTAT |
| 622 | ACTTCCAAAGACTTCGTA |
| 623 | ATCCAGCCAAACACTCCC |
| 624 | CATCCAGCCAAACACTCC |
| 625 | ACATCCAGCCAAACACTC |
| 626 | AACATCCAGCCAAACACT |
| 627 | CACATTCATAGAGCCCACA |
| 628 | GCACATTCATAGAGCCCAC |
| 629 | AGCACATTCATAGAGCCCA |
| 630 | CAACTAAAGCACCCATATA |
| 631 | TCAACTAAAGCACCCATAT |
| 632 | ATCAACTAAAGCACCCATA |
| 633 | ACTTCCACAAGATAACCCT |
| 634 | CACTTCCACAAGATAACCC |
| 635 | CCACTTCCACAAGATAACC |
| 636 | CCCACTTCCACAAGATAAC |
| 637 | TCCCACTTCCACAAGATAA |
| 638 | ATCCCACTTCCACAAGATA |
| 639 | CATCCCACTTCCACAAGAT |
| 640 | AGAATAAGCTCTCAACTGT |
| 641 | GTTACAGGAGAATAAGCTC |
| 642 | TTCAAGGATGGTTACAGGA |
| 643 | ATTCAAGGATGGTTACAGG |
| 644 | TCTCCACTCACCATGAACA |
| 645 | ACTCTCCACTCACCATGAA |
| 646 | AACTCTCCACTCACCATGA |
| 647 | AAACTCTCCACTCACCATG |
| 648 | ACATACATTTCACCCGAAG |
| 649 | TACATACATTTCACCCGAA |
| 650 | CTACATACATTTCACCCGA |
| 651 | GAGCCCATCACAGCGAAAG |
| 652 | ATCATATAAGAACTACCGG |
| 653 | CCGTAAGACAGCTCTTCCT |
| 654 | TCCGTAAGACAGCTCTTCC |
| 655 | GGTCCCAGGTAAGTTAATT |
| 656 | TTGATCTCATTCGGAAGGA |
| 657 | CTTGATCTCATTCGGAAGG |
| 658 | TGCAGCCTAAGTTTATTAG |
| 659 | CATATGTTAAGGATGACTG |
| 660 | TATCAATCAGTGTGGAGCT |
| | T |
| 661 | GTATCAATCAGTGTGGAGC |
| | T |
| 662 | AGTATCAATCAGTGTGGAG |
| | C |
| 663 | GAGTATCAATCAGTGTGGA |
| | G |
| 664 | AGAGTATCAATCAGTGTGG |
| | A |
| 665 | CATCCCACTTCCACAAGAT |
| | A |
| 666 | TCATCCCACTTCCACAAGA |
| | T |
| 667 | CACACCCTTGGTCAGAACT |
| | T |
| 668 | ACACACCCTTGGTCAGAAC |
| | T |
| 669 | CACACACCCTTGGTCAGAA |
| | C |
| 670 | TCACACACCCTTGGTCAGA |
| | A |
| 671 | GTCACACACCCTTGGTCAG |
| | A |
| 672 | CGTCACACACCCTTGGTCA |
| | G |
| 673 | ACGTCACACACCCTTGGTC |
| | A |
| 674 | CACGTCACACACCCTTGGT |
| | C |
| 675 | CCACGTCACACACCCTTGG |
| | T |
| 676 | CCCACGTCACACACCCTTG |
| | G |
| 677 | GCCCACGTCACACACCCTT |
| | G |
| 678 | ATCAAGCTCTGTATCGCAA |
| | A |
| 679 | CATCAAGCTCTGTATCGCA |
| | A |
| 680 | CCATCAAGCTCTGTATCGC |
| | A |
| 681 | TTGTAGGAGCAGCATAGGC |
| | A |
| 682 | TTTGTAGGAGCAGCATAGG |
| | C |
| 683 | CTGACTAACATTTGGGCTA |
| | G |
| 684 | GAACATGAGTCTAAATGAA |
| | C |
| 685 | CTGAACATGAGTCTAAATG |
| | A |
| 686 | CCTAGTGGCTCAATCAGTT |
| | A |
| 687 | ACCTAGTGGCTCAATCAGT |
| | T |
| 688 | TACCTAGTGGCTCAATCAG |
| | T |
| 689 | ATACCTAGTGGCTCAATCA |
| | G |
| 690 | AAACGCAACTGAACTGGAT |
| | C |
| 691 | AGAGATCAGTCACCCAACA |
| | G |
| 692 | CCTGCAAGCCTAAGATGAA |
| | A |
| 693 | GATCAGCATAACTAGAGAT |
| | T |
| 694 | ATAGTCAATCAGTGCCCTA |
| | A |

TABLE 6
ASO sequences targeting human
ENSG00000203585/SHARED_00113753
(Chr12)

| SEQ | |
|---|---|
| ID | |
| NO. | Sequence |
| 695 | GGACCTAGCCAATAAG |
| 696 | TAGCATAATCTGGCAC |
| 697 | TCCTAGCATAATCTGG |
| 698 | ACATCCTAGCATAATC |
| 699 | ACGCCAGCTCTCAACT |
| 700 | GACGCCAGCTCTCAAC |
| 701 | TGACGCCAGCTCTCAA |
| 702 | ATGACGCCAGCTCTCA |
| 703 | AATGACGCCAGCTCTC |
| 704 | TAATGACGCCAGCTCT |
| 705 | CTAATGACGCCAGCTC |
| 706 | TCTAATGACGCCAGCT |
| 707 | TTCTAATGACGCCAGC |
| 708 | GTTCTAATGACGCCAG |
| 709 | TGTTCTAATGACGCCA |
| 710 | CTGTTCTAATGACGCC |
| 711 | CCTGTTCTAATGACGC |
| 712 | GCCTGTTCTAATGACG |
| 713 | CTGCCGAGACATGCCT |
| 714 | TCCTGCCGAGACATGC |
| 715 | TTCCTGCCGAGACATG |
| 716 | CTTCCTGCCGAGACAT |
| 717 | ACCTTCCTGCCGAGAC |
| 718 | GACCTTCCTGCCGAGA |
| 719 | CACATAGCCTCAGCGA |
| 720 | TCACATAGCCTCAGCG |
| 721 | GTCCCAATTTGAAGCC |
| 722 | CAAACGGTGAGTTTCA |
| 723 | CCAAACGGTGAGTTTC |
| 724 | GATTAGTATGCCAGGG |
| 725 | TGATTAGTATGCCAGG |
| 726 | GATCATTGCATAGAGC |
| 727 | GTAGATCATTGCATAG |
| 728 | CGCTCAAATAATGTGC |
| 729 | ACGCTCAAATAATGTG |
| 730 | CACTAACGCTCAAATA |
| 731 | TCACTAACGCTCAAAT |
| 732 | ATCACTAACGCTCAAA |
| 733 | TATCACTAACGCTCAA |
| 734 | TTATCACTAACGCTCA |
| 735 | ACTTATCACTAACGCT |
| 736 | CACTTATCACTAACGC |
| 737 | CCCATAGACATAGGTC |
| 738 | ACCCATAGACATAGGT |
| 739 | GCTATAACTGATTCCC |
| 740 | CGTAACAGCTCAGACA |
| 741 | CCGTAACAGCTCAGAC |
| 742 | TATATATCTAGGACCC |
| 743 | CTATATATCTAGGACC |
| 744 | CCTAACGACTAAAGAA |
| 745 | CTCCTAACGACTAAAG |
| 746 | CCTCCTAACGACTAAA |
| 747 | GCCTCCTAACGACTAA |
| 748 | TGCCTCCTAACGACTA |
| 749 | ATGCCTCCTAACGACT |
| 750 | AATGCCTCCTAACGAC |
| 751 | AAATGCCTCCTAACGA |
| 752 | CAAATGCCTCCTAACG |
| 753 | GTTCTATCAGGCTCAC |
| 754 | TAGTTCTATCAGGCTC |
| 755 | GGTAAGGAATACACTC |
| 756 | AACATCGCAGTTGTTC |
| 757 | TAACATCGCAGTTGTT |
| 758 | GATAACATCGCAGTTG |
| 759 | GGATAACATCGCAGTT |
| 760 | AGGATAACATCGCAGT |
| 761 | TAGAGGATAACATCGC |
| 762 | TTAGAGGATAACATCG |
| 763 | AGCCAGGACTCTACGG |
| 764 | CAGCCAGGACTCTACG |
| 765 | AAACCACGCACCAGCC |
| 766 | CGATTCCACTTCAGCA |
| 767 | TTAACCACGATTCCAC |
| 768 | GTTAACCACGATTCCA |
| 769 | TGTTAACCACGATTCC |
| 770 | GCCTGTTAACCACGAT |
| 771 | CCGTGGTAATTTGAAA |
| 772 | CGTAAGAAAGCACTAT |
| 773 | CGTGTGTATCTCCTAG |
| 774 | GCGTGTGTATCTCCTA |
| 775 | CGCAACTTCAATATCC |
| 776 | ACGCAACTTCAATATC |
| 777 | CACGCAACTTCAATAT |
| 778 | CCACGCAACTTCAATA |
| 779 | GCCACGCAACTTCAAT |
| 780 | TGCCACGCAACTTCAA |
| 781 | ATCTGCCACGCAACTT |
| 782 | CATCTGCCACGCAACT |
| 783 | AGGCTAATAGGTTTAG |
| 784 | CGATTCACATTGACTT |
| 785 | TCGATTCACATTGACT |
| 786 | ATCGATTCACATTGAC |
| 787 | AGAACCTAAGATCGAT |
| 788 | TATGATGTGCCCATAC |
| 789 | TCCAAACTTAGTCAGC |
| 790 | GTCCAAACTTAGTCAG |
| 791 | GTACGATAAACTGTGA |
| 792 | GGTACGATAAACTGTG |
| 793 | AGGTACGATAAACTGT |
| 794 | CCTAGACATTGACCGG |
| 795 | TCCTAGACATTGACCG |
| 796 | TATGATGTGTAACCTC |
| 797 | CCTATGATGTGTAACC |
| 798 | ACCTATGATGTGTAAC |
| 799 | CTGATAAGTGGTATCC |
| 800 | TCACATAGGTATAGCC |
| 801 | GTTCACATAGGTATAG |
| 802 | GGTTCACATAGGTATA |
| 803 | GTTGGTTCACATAGGT |
| 804 | AGTTGGTTCACATAGG |
| 805 | GAGTCTTAACTATACC |
| 806 | GGAGTCTTAACTATAC |
| 807 | TCGTGTTAGGTTTGAG |
| 808 | ATCGTGTTAGGTTTGA |
| 809 | AATCGTGTTAGGTTTG |
| 810 | TCCTTAATCGTGTTAG |
| 811 | GTCCTTAATCGTGTTA |
| 812 | ATGGTGGTGTACTTGC |
| 813 | AGTCCTCTCGGCAAGA |
| 814 | AACCATTAGAGTCCTC |
| 815 | GAAACCATTAGAGTCC |
| 816 | CACGCAGAGATCAACA |
| 817 | TCACGCAGAGATCAAC |
| 818 | GTCACGCAGAGATCAA |
| 819 | AGTCACGCAGAGATCA |
| 820 | CAGTCACGCAGAGATC |
| 821 | TGATCTGGACGATGAG |
| 822 | AAAGTTGATCTGGACG |
| 823 | TAGGTTGGTCTAGGAA |
| 824 | TTAGGTTGGTCTAGGA |
| 825 | TCGAACTTAGCTTATG |
| 826 | GTACCCACCCTTACAA |
| 827 | GCTGACCCATAATAGG |
| 828 | CGCTGACCCATAATAG |
| 829 | TCGCTGACCCATAATA |
| 830 | ATGATAGAGTCGCTGA |
| 831 | AATGATAGAGTCGCTG |
| 832 | AAATGATAGAGTCGCT |
| 833 | CCGAAATGATAGAGTC |
| 834 | TCCGAAATGATAGAGT |
| 835 | TTCCGAAATGATAGAG |
| 836 | CTTTCCGAAATGATAG |
| 837 | GTTATATGCTTAGAGG |
| 838 | ACTAGAACCTTGACCC |
| 839 | TATCCCAGGTCACTAG |
| 840 | CTGAGAGTCATATCCC |
| 841 | CGCCACTGAGAGTCAT |
| 842 | ACGCCACTGAGAGTCA |
| 843 | TACGCCACTGAGAGTC |
| 844 | ATACGCCACTGAGAGT |
| 845 | CATACGCCACTGAGAG |
| 846 | ACATACGCCACTGAGA |
| 847 | AACATACGCCACTGAG |
| 848 | ATAAACATACGCCACT |
| 849 | CATAAACATACGCCAC |
| 850 | GACATAAACATACGCC |
| 851 | GATCCAATTAGAGTCC |
| 852 | TGATCCAATTAGAGTC |
| 853 | TGCCTTGGACTGATCC |
| 854 | ATGCCTTGGACTGATC |
| 855 | GATGCCTTGGACTGAT |
| 856 | TAGATAGGTGAGATCT |
| 857 | TTAGATAGGTGAGATC |
| 858 | ATCTCCGTGGTTTGAT |
| 859 | AATCTCCGTGGTTTGA |
| 860 | ACCTAATCTCCGTGGT |
| 861 | TACCTAATCTCCGTGG |
| 862 | GTACCTAATCTCCGTG |
| 863 | GTGGTAGCAAGTCTTA |
| 864 | GGTGGTAGCAAGTCTT |
| 865 | GCACCCTAACTATTTG |
| 866 | GGCACCCTAACTATTT |
| 867 | TGGCACCCTAACTATT |
| 868 | TTGGCACCCTAACTAT |
| 869 | TTTGGCACCCTAACTA |
| 870 | CTGAGTAGGACATTGG |
| 871 | CTCGACTAATCAGAAT |
| 872 | CCTCGACTAATCAGAA |
| 873 | CCCTCGACTAATCAGA |
| 874 | ACCCTCGACTAATCAG |
| 875 | CACCCTCGACTAATCA |
| 876 | ACACCCTCGACTAATC |
| 877 | AACACCCTCGACTAAT |
| 878 | AAACACCCTCGACTAA |
| 879 | CAAACACCCTCGACTA |
| 880 | TCAAACACCCTCGACT |
| 881 | ATCAAACACCCTCGAC |
| 882 | AATCAAACACCCTCGA |
| 883 | GAATCAAACACCCTCG |
| 884 | CGGTGATGTTGTGTTT |
| 885 | TCGGTGATGTTGTGTT |
| 886 | TTCGGTGATGTTGTGT |
| 887 | TTTCGGTGATGTTGTG |
| 888 | CTTTCGGTGATGTTGT |
| 889 | GCTTTCGGTGATGTTG |
| 890 | AGCTTTCGGTGATGTT |
| 891 | CAGCTTTCGGTGATGT |
| 892 | CCAGCTTTCGGTGATG |
| 893 | TCATGCCAGCTTTCGG |
| 894 | CATCTTGGGTATTTCG |
| 895 | GTAACCCATATCTGTG |
| 896 | GTATCACCTCAACACC |
| 897 | GAGCCTTGTCATCAAT |
| 898 | CTAGTGAGCCTTGTCA |
| 899 | CCTAGTGAGCCTTGTC |
| 900 | ACCTAGTGAGCCTTGT |
| 901 | TACCTAGTGAGCCTTG |
| 902 | ATACCTAGTGAGCCTT |
| 903 | TATACCTAGTGAGCCT |
| 904 | GTATACCTAGTGAGCC |
| 905 | GCACACATAGTTAGAG |
| 906 | TAGTATCAGTCCAGGG |
| 907 | CTAGTATCAGTCCAGG |
| 908 | ATCCATACTTCGGTGC |
| 909 | AATCCATACTTCGGTG |
| 910 | CAATCCATACTTCGGT |
| 911 | GCAATCCATACTTCGG |
| 912 | TGCAATCCATACTTCG |
| 913 | GTGCAATCCATACTTC |
| 914 | GATTCAACAGTCTAGG |
| 915 | CAATATAGATCACGGT |
| 916 | TCAATATAGATCACGG |
| 917 | CCCTTCAATATAGATC |
| 918 | GTATTGTCCATACTGC |
| 919 | GTGTATTGTCCATACT |
| 920 | AGTGTATTGTCCATAC |
| 921 | ATCTAGTGTATTGTCC |
| 922 | CATCTAGTGTATTGTC |
| 923 | GACACAACATCTAGTG |
| 924 | TAGTCTACTCACTGGT |
| 925 | ATAGTCTACTCACTGG |
| 926 | GCAATAGTCTACTCAC |
| 927 | GCTGCAATAGTCTACT |
| 928 | GGAGGTGCAATCACGC |
| 929 | ACGTCCTCAATCTCAA |
| 930 | AAATCCTACCCTACGT |
| 931 | GTATCCATTCCACCGG |
| 932 | GCTACCAGAAGAGTCC |
| 933 | ATTGGGATGATTAGCT |
| 934 | TATTGGGATGATTAGC |
| 935 | GTCTGCATTAGAGTGG |
| 936 | TGGAACGTTAATTGGA |
| 937 | ATGGAACGTTAATTGG |
| 938 | CTGAGGAAACGTTATA |
| 939 | ACCAGTATGACTCTTG |
| 940 | TTTGATAACCTCGAAC |
| 941 | CGAGTTAGCCACCATT |
| 942 | CCGAGTTAGCCACCAT |
| 943 | CCCGAGTTAGCCACCA |
| 944 | ACCCGAGTTAGCCACC |
| 945 | CACCCGAGTTAGCCAC |
| 946 | TCACCCGAGTTAGCCA |
| 947 | TTCACCCGAGTTAGCC |
| 948 | ATTCACCCGAGTTAGC |
| 949 | TAGGCTGGTATTGTGT |
| 950 | ATAGGCTGGTATTGTG |
| 951 | CATAGGCTGGTATTGT |
| 952 | CGAGACCACAACATAG |
| 953 | GCGAGACCACAACATA |
| 954 | TGCGAGACCACAACAT |
| 955 | ATGCGAGACCACAACA |
| 956 | CATGCGAGACCACAAC |
| 957 | CACGTAGCTTGGGAAG |
| 958 | ATCACGTAGCTTGGGA |
| 959 | AATCACGTAGCTTGGG |
| 960 | AAATCACGTAGCTTGG |
| 961 | GGAAATCACGTAGCTT |
| 962 | GGGAAATCACGTAGCT |
| 963 | TTAGGCATAATGGTTG |
| 964 | GGGCATAATAACTGAC |
| 965 | CGAAGATTGCCTCATG |
| 966 | TCGAAGATTGCCTCAT |
| 967 | ATCGAAGATTGCCTCA |
| 968 | GATCGAAGATTGCCTC |
| 969 | TCTGAATGGATCGAAG |
| 970 | GTCTGAATGGATCGAA |
| 971 | TGTCTGAATGGATCGA |
| 972 | TTGTCTGAATGGATCG |
| 973 | GAACCACCTCCTTAGT |
| 974 | GGATGATACCTGATTC |
| 975 | CGTTGAATTACTGGGA |
| 976 | CCTAAGAAACGTTGAA |
| 977 | TCCTAAGAAACGTTGA |
| 978 | ATCAATGGGAGTGCTA |
| 979 | CCCTCCACGAAATTAA |
| 980 | GCCCTCCACGAAATTA |
| 981 | TGCCCTCCACGAAATT |
| 982 | ATGCCCTCCACGAAAT |
| 983 | GATGCCCTCCACGAAA |
| 984 | AGATGCCCTCCACGAA |
| 985 | TCGTACTTGGAAGCCA |
| 986 | ATCGTACTTGGAAGCC |
| 987 | GATCGTACTTGGAAGC |
| 988 | AGATCGTACTTGGAAG |
| 989 | TAGATCGTACTTGGAA |
| 990 | ATAGATCGTACTTGGA |
| 991 | CATAGATCGTACTTGG |
| 992 | CCATAGATCGTACTTG |
| 993 | ACCATAGATCGTACTT |
| 994 | AACCATAGATCGTACT |
| 995 | GAACCATAGATCGTAC |
| 996 | GGAACCATAGATCGTA |
| 997 | GATTGTGATGTGCGAG |
| 998 | ACGTTTATTCCTGTAG |
| 999 | GTGAATTGTAAGTCGT |
| 1000 | CCGTGAATTGTAAGTC |
| 1001 | ACCGTGAATTGTAAGT |
| 1002 | GACCGTGAATTGTAAG |
| 1003 | GGACCGTGAATTGTAA |
| 1004 | TGGACCGTGAATTGTA |
| 1005 | TTGGACCGTGAATTGT |
| 1006 | TTTGGACCGTGAATTG |
| 1007 | TGCACATCTCGCTGGT |
| 1008 | ATGCACATCTCGCTGG |
| 1009 | TATGCACATCTCGCTG |
| 1010 | ATATGCACATCTCGCT |
| 1011 | CATATGCACATCTCGC |
| 1012 | TCATATGCACATCTCG |
| 1013 | GTATCATTGTTCAGCC |
| 1014 | ACCATACCTGTGTATC |
| 1015 | GGTTAGGTAGCATCAC |
| 1016 | AAGGTTAGGTAGCATC |
| 1017 | CAAGGTTAGGTAGCAT |
| 1018 | GGCTGTTGATGATTGC |
| 1019 | TACGATTCAAAGCCAC |
| 1020 | CTTACGATTCAAAGCC |
| 1021 | ACTTACGATTCAAAGC |
| 1022 | TTCCGAGTAAGATTTG |
| 1023 | CATTTCCGAGTAAGAT |
| 1024 | AGATGGATACCTTAGG |
| 1025 | ACGACCAACAGCTCTG |
| 1026 | AACGACCAACAGCTCT |
| 1027 | AAACGACCAACAGCTC |
| 1028 | CCAAACGACCAACAGC |
| 1029 | ACCAAACGACCAACAG |
| 1030 | CACCAAACGACCAACA |
| 1031 | CCACCAAACGACCAAC |
| 1032 | GCCACCAAACGACCAA |
| 1033 | AGAATCGTTTGTCTTG |
| 1034 | CTCAGAATCGTTTGTC |
| 1035 | ACTCAGAATCGTTTGT |
| 1036 | AACTCAGAATCGTTTG |
| 1037 | CGACTAGAGCCACATC |
| 1038 | CCGACTAGAGCCACAT |
| 1039 | ACCGACTAGAGCCACA |
| 1040 | GATATGAGCTATGGAC |
| 1041 | CTTCATTGACCGTGAG |
| 1042 | ACTTCATTGACCGTGA |
| 1043 | AACTTCATTGACCGTG |
| 1044 | AAACTTCATTGACCGT |
| 1045 | TAAACTTCATTGACCG |
| 1046 | CGTGAGATGTTAATAG |
| 1047 | CCGTGAGATGTTAATA |
| 1048 | CCCGTGAGATGTTAAT |
| 1049 | ACATCCTAGCATAATCT |
| 1050 | GTAACACCCAGCATCTG |
| 1051 | TGTAACACCCAGCATCT |
| 1052 | ATGATGTTTAGATGCTC |
| 1053 | GAACTTAGCTTATGATG |
| 1054 | AACGTTGAATTACTGGG |
| 1055 | AAACGTTGAATTACTGG |
| 1056 | AGGGCACATGTTGGATT |
| 1057 | AAGGGCACATGTTGGAT |
| 1058 | GCACCAGCCAGGACTCTA |
| 1059 | AAACCACGCACCAGCCAG |
| 1060 | CAGCCTTCAAGTAAATTC |
| 1061 | CTAATTCAGCCTTCAAGT |
| 1062 | GATACCTTCAGTCCCAAG |
| 1063 | TGATACCTTCAGTCCCAA |
| 1064 | TTGGTTCACATAGGTATAG |
| 1065 | GTTGGTTCACATAGGTATA |
| 1066 | TGAGGAGACAGCAATTCAC |
| 1067 | TTGAGGAGACAGCAATTCA |
| 1068 | GATGTGTAACCTCAATTTCT |
| 1069 | ACCTATGATGTGTAACCTCA |
| 1070 | GTACAGAATCCAATGACAGA |
| 1071 | AGTACAGAATCCAATGACAG |
| 1072 | AAACACCTTGTTCGCTTTGG |
| 1073 | GTCTCTCATTACAAATGGTG |
| 1074 | CTGCCGCTTGTAAACC |
| 1075 | ACTGCCGCTTGTAAAC |
| 1076 | AACTGCCGCTTGTAAA |
| 1077 | AAACTGCCGCTTGTAA |
| 1078 | GGAAACTGCCGCTTGT |
| 1079 | ATGAAGACACACCCGG |
| 1080 | CGACTAGAGCCACATC |
| 1081 | CCGACTAGAGCCACAT |
| 1082 | ACCGACTAGAGCCACA |
| 1083 | AACCGACTAGAGCCAC |
| 1084 | AAACCGACTAGAGCCA |
| 1085 | TAAACCGACTAGAGCC |
| 1086 | CTAAACCGACTAGAGC |
| 1087 | ACTAAACCGACTAGAG |
| 1088 | GACTAAACCGACTAGA |
| 1089 | GATTACCCACTTCAAG |
| 1090 | GAGCTATGTCATGTCC |
| 1091 | GACTTTGCACTTCGTA |
| 1092 | AGTTAGAGTTAGGCGT |
| 1093 | GACACCTCAAGTTAGA |
| 1094 | TGACACCTCAAGTTAG |
| 1095 | GTCTAGGTTGCACGTT |
| 1096 | GGTCTAGGTTGCACGT |
| 1097 | AACCTGCTAGATCTTG |
| 1098 | CAACCTGCTAGATCTT |
| 1099 | TCAACCTGCTAGATCT |
| 1100 | GAACACACTTCGAGAG |
| 1101 | AGCAGAACACACTTCG |
| 1102 | CGCTGTTCCATTAACA |
| 1103 | TAGTCTTAATCCTGGC |
| 1104 | CAAGGGATGATAGCTC |
| 1105 | GGATCACACTAAACCC |
| 1106 | CCAATAACCCATTGCC |
| 1107 | GCCAATAACCCATTGC |
| 1108 | TACCTTGCCAATAACC |
| 1109 | GTACCTTGCCAATAAC |
| 1110 | CACCCTAATAGTGTTG |
| 1111 | GTCTCACCCTAATAGT |
| 1112 | ACTTATGTGGCTCCGG |
| 1113 | CGGTTATACAGATGCT |
| 1114 | AGGCATGGATTATACC |
| 1115 | TAGGCATGGATTATAC |
| 1116 | ACCAGAGATCGCCCTC |
| 1117 | TACCAGAGATCGCCCT |
| 1118 | ATACCAGAGATCGCCC |
| 1119 | TATCCGTCAGGCAAAG |
| 1120 | GTATCCGTCAGGCAAA |
| 1121 | GAGATAGATTCTTCGT |
| 1122 | AGGCTACATTACCTCC |
| 1123 | CTAGGCTACATTACCT |
| 1124 | ACTAGGCTACATTACC |
| 1125 | CACTAGGCTACATTAC |
| 1126 | TCGTGTTTATTGCTGG |
| 1127 | GTTGATTGTGATCCAG |
| 1128 | GTTTGTGACGCCTGCC |
| 1129 | AGTTTGTGACGCCTGC |
| 1130 | AAGTTTGTGACGCCTG |
| 1131 | AAAGTTTGTGACGCCT |
| 1132 | GAGCCCATCTGATTGC |
| 1133 | CGTGACTCCAGAATGT |
| 1134 | CCGTGACTCCAGAATG |
| 1135 | ATGATCCTCCGTGACT |
| 1136 | GATGATCCTCCGTGAC |
| 1137 | GATTGAACACGTTGTT |
| 1138 | TGATTGAACACGTTGT |
| 1139 | TTGATTGAACACGTTG |
| 1140 | GTTGATTGAACACGTT |
| 1141 | AGTTGATTGAACACGT |
| 1142 | TAGTTGATTGAACACG |
| 1143 | GTAGTTGATTGAACAC |
| 1144 | AGGTAGTTGTAGGCTT |
| 1145 | GAGGTAGTTGTAGGCT |
| 1146 | GCCAGTCAATAATCCC |
| 1147 | GACCTTAGAGCCAGTC |
| 1148 | CCCTAGAATAAGTACC |
| 1149 | GCCCTAGAATAAGTAC |
| 1150 | ACTCTGCGTTAGGAAT |
| 1151 | CACTCTGCGTTAGGAA |
| 1152 | ACAGCCGACTCAAAGC |
| 1153 | TTACAGCCGACTCAAA |
| 1154 | AAGAGCATAACGTACC |
| 1155 | TAAGAGCATAACGTAC |
| 1156 | CTAAGAGCATAACGTA |
| 1157 | ACTAAGAGCATAACGT |
| 1158 | TACTAAGAGCATAACG |
| 1159 | CGCCCTGAAACCCAAC |
| 1160 | ATGAAACATCAATCCAG |
| 1161 | ATTAGCCATTCCCAGCC |
| 1162 | CAGCTTAAGGATGTATT |
| 1163 | TAGAACAGCTTAAGGAT |
| 1164 | GTTGACTCTGCCAATTA |
| 1165 | TGTTGACTCTGCCAATT |
| 1166 | GCCAATAACCCATTGCCA |
| 1167 | TGCCAATAACCCATTGCC |
| 1168 | TACCTAGTGACCCAATGC |
| 1169 | GTACCTAGTGACCCAATG |
| 1170 | TAAACACACTCGCTCACT |
| 1171 | ATAAACACACTCGCTCAC |
| 1172 | TTTACCTCGAATCAAGCT |
| 1173 | AAGTCATCTTCTGGTTGT |
| 1174 | ACATTTCAAGAAGCTACG |
| 1175 | CTAATGAGCATAGCCAGG |
| 1176 | CGACTAGAGCCACATCAGC |
| 1177 | AAACACCTTGTTCGCTTTG |
| 1178 | TCTCTCATTACAAATGGTG |
| 1179 | TAAACAGGTAAAGCTATCC |
| 1180 | CTTCCATCAGTATAAGATC |
| 1181 | GTACATACAGCTTAGATCA |
| 1182 | AGTACATACAGCTTAGATC |
| 1183 | GTATCAGCCTCTGTGAAAC |
| 1184 | TGTATCAGCCTCTGTGAAA |
| 1185 | AACTTACACATCTGCTTGA |
| 1186 | TAACTTACACATCTGCTTG |
| 1187 | GCTAAATTCTATTCAACCTG |
| 1188 | AAAGCTTCTCCTCTCATTGG |
| 1189 | GGCACAAATTAGAACAGCTT |
| 1190 | GGGCACAAATTAGAACAGCT |
| 1191 | ATCTGGTAACATCCTATGGC |
| 1192 | TAAAGATAGCACCATGCCAT |
| 1193 | TGTCATCTTTAAGCTACAGA |
| 1194 | TCTGTCATCTTTAAGCTACA |
| 1195 | CTTCAGTGTGGTGTTTGTAT |
| 1196 | TGATTTGACCCTGTAATTGC |
| 1197 | AAGCACATTAACACCCACTG |
| 1198 | GAAGCACATTAACACCCACT |

TABLE 7
ASO sequences targeting human
ENSG00000288903 (Chr9)

| SEQ | |
|---|---|
| ID | |
| NO. | Sequence |
| 1199 | GCCAAGGTTAGTGAGC |
| 1200 | GTCTAAAGATTGGGAT |
| 1201 | GCCTTAATCAGTTGTC |
| 1202 | CCATAACTTGATTAGC |
| 1203 | GCATGTTGGTCCCTAC |
| 1204 | TTGGACAACACTAGGG |
| 1205 | AGCACTAAGCGTCTTG |
| 1206 | AAGAGCACTAAGCGTC |
| 1207 | AGCAGCAAACGTCCTG |
| 1208 | CAGCAGCAAACGTCCT |
| 1209 | ACCCAGTAGCAAGTTG |
| 1210 | TCAATTACACCTGATC |
| 1211 | GGACACCTTAACTGCA |
| 1212 | CCCTACTGATGATCTG |
| 1213 | GTACCTAAGTCATCAG |
| 1214 | GGAAGTACCTAAGTCA |
| 1215 | GACTAACTACAGGAGT |
| 1216 | TGACTAACTACAGGAG |
| 1217 | GTTGACTAACTACAGG |
| 1218 | GGATGCACAACTTGAT |
| 1219 | AGCAATCCAAGCGTGC |
| 1220 | AAGCAATCCAAGCGTG |
| 1221 | GCAATAGCCGAAACAT |
| 1222 | GTATATCAGACCCTCA |
| 1223 | TGTATATCAGACCCTC |
| 1224 | GCTTAATTCTTCGGTG |
| 1225 | GGCTTAATTCTTCGGT |
| 1226 | GGGCTTAATTCTTCGG |
| 1227 | GCAATGTCCAATGACC |
| 1228 | GTGCAATGTCCAATGA |
| 1229 | CCTAAGTCATCGGAGA |
| 1230 | ACCTAAGTCATCGGAG |
| 1231 | TACCTAAGTCATCGGA |
| 1232 | GTACCTAAGTCATCGG |
| 1233 | AGTACCTAAGTCATCG |
| 1234 | GGAAGTACCTAAGTCA |
| 1235 | GACCCTGTAATTAGTC |
| 1236 | GTTCACATAGAGTATC |
| 1237 | GCATACACTCCCAATA |
| 1238 | AGTCTATCCAATCATG |
| 1239 | AGTGACCTACTTATCC |
| 1240 | GTTAGCTCACCAACAC |
| 1241 | TGTTAGCTCACCAACA |
| 1242 | TTGTTAGCTCACCAAC |
| 1243 | GGTTCATCCTGGTCAC |
| 1244 | TACTAAGACCCACAAC |
| 1245 | CCTTACTAAGACCCAC |
| 1246 | GCCATCCTATAACTAC |
| 1247 | TAAGCCATCCTATAAC |
| 1248 | GCCCTAATCAAGTAGA |
| 1249 | AGCCCTAATCAAGTAG |
| 1250 | GAATCTCTTATGGGTA |
| 1251 | TAACTCTCTAGTACAC |
| 1252 | CATAACTCTCTAGTAC |
| 1253 | GACACCTGATGTACAC |
| 1254 | GGACACCTGATGTACA |
| 1255 | AGGACACCTGATGTAC |
| 1256 | GACATGCCCTATGACA |
| 1257 | TCTATCAGAGGAACCC |
| 1258 | TATTGTCAGGCTACTC |
| 1259 | GTTACATCAACCCATC |
| 1260 | GCCAAACCCTAGTCTG |
| 1261 | GGCTTAATCCCTGAAC |
| 1262 | GATCCCAATTTAATCC |
| 1263 | TTGATGAGGTTAGATC |
| 1264 | TGATACATGAGTACGA |
| 1265 | CTGATACATGAGTACG |
| 1266 | GTAACTCAGCCTATGC |
| 1267 | ATCTTCATTGTGGGCC |
| 1268 | GGATCAGCCAATTTGC |
| 1269 | GAACCAGCCTACAGTT |
| 1270 | TTGAGAACCAGCCTAC |
| 1271 | TGATTCCCTGATAACC |
| 1272 | TGCTTAGACATAATCC |
| 1273 | GGTTGAAGATGCTTAG |
| 1274 | GCATGACGCAATATAA |
| 1275 | AAGCATGACGCAATAT |
| 1276 | GGATTACTAAATTGGG |
| 1277 | CTAGTCAGCTCAGATA |
| 1278 | GTTTGACATAACCAGT |
| 1279 | GATCCGTAAAGCATGG |
| 1280 | GTCTTGCCAGTCAGCT |
| 1281 | GGATTGCCAACTGTGA |
| 1282 | GGCAGCAATAAGGTTC |
| 1283 | GGTCAAGCTCCTCTAC |
| 1284 | CGGTCAAGCTCCTCTA |
| 1285 | CAATGGCATCGCTGGT |
| 1286 | ACCTGATATAGTCACC |
| 1287 | GCTACCTGATATAGTC |
| 1288 | GGCTACCTGATATAGT |
| 1289 | TTACTGAATACGGGCC |
| 1290 | TTTACTGAATACGGGC |
| 1291 | GATTTACTGAATACGG |
| 1292 | GTGATAATCCCTCAGC |
| 1293 | GACAAGCTCACTAAGG |
| 1294 | GCTAACACATAAGGGT |
| 1295 | GTGTACTCAACTCCAC |
| 1296 | TTATGGGCAGTCAGTC |
| 1297 | GTATTATGGGCAGTCA |
| 1298 | TGTATTATGGGCAGTC |
| 1299 | CTTCTTAGCATAAGCG |
| 1300 | ATGATAGTCCCTCTGG |
| 1301 | AACATGATAGTCCCTC |
| 1302 | GTAACATGATAGTCCC |
| 1303 | AGTAACATGATAGTCC |
| 1304 | TTTGGTAAACTGGGCC |
| 1305 | GTAACTACATCAGGTA |
| 1306 | GTCCAAGTTACTGATC |
| 1307 | GCAATTAGGTAGAGTT |
| 1308 | CCGGCAATTAGGTAGA |
| 1309 | CTTTATCCAAAGCCGG |
| 1310 | TCTTTATCCAAAGCCG |
| 1311 | ACTAGATTGTGTATGC |
| 1312 | GACCTTTACTAGATTG |
| 1313 | GTGACCTTTACTAGAT |
| 1314 | AATGTATAGGGCTTGC |
| 1315 | GTCCCATAACCCATGC |
| 1316 | AGTCCCATAACCCATG |
| 1317 | ATGAAGTCCCATAACC |
| 1318 | CAATCAGGCTCTAAGC |
| 1319 | GTATTACCAGCCATCA |
| 1320 | CAAGCTACAAGATTGC |
| 1321 | GTCCTATGATACCAAC |
| 1322 | GAGTCCTATGATACCA |
| 1323 | AGAGTCCTATGATACC |
| 1324 | AAGAGTCCTATGATAC |
| 1325 | CTTACAGGGTGGACAT |
| 1326 | ATCTTACAGGGTGGAC |
| 1327 | GAATAGCCTAGCTCTG |
| 1328 | GGAATAGCCTAGCTCT |
| 1329 | GCCTGGACCGTGATCA |
| 1330 | CTCTAGTTGTGAAGTG |
| 1331 | TACCAGTACTTACAGC |
| 1332 | AAGCTCCAACGTTAAC |
| 1333 | GAAGCTCCAACGTTAA |
| 1334 | TAGGAGGTTAGTTAAC |
| 1335 | TTGTAGGAGGTTAGTT |
| 1336 | ATTGTAGGAGGTTAGT |
| 1337 | TGATTGTAGGAGGTTA |
| 1338 | GTGATTGTAGGAGGTT |
| 1339 | TTAGGTAAGCTGTGAC |
| 1340 | CCCTCAGCATAACTAC |
| 1341 | CGACAACTTGCCTCCA |
| 1342 | ACCGACAACTTGCCTC |
| 1343 | CACCGACAACTTGCCT |
| 1344 | ACACCGACAACTTGCC |
| 1345 | AACACCGACAACTTGC |
| 1346 | TAGCAACCATATAGCT |
| 1347 | TTAGCAACCATATAGC |
| 1348 | CATTAGTTGTGTTAGC |
| 1349 | GGAGACCTAACAGTTC |
| 1350 | TGGGACATTTAGGGTC |
| 1351 | GTCAACTAAGCACTGG |
| 1352 | AGACACAGCATCGATC |
| 1353 | TAGACACAGCATCGAT |
| 1354 | TTAGACACAGCATCGA |
| 1355 | GTTAGACACAGCATCG |
| 1356 | GGTTAGACACAGCATC |
| 1357 | GCAGCCTACAAGACTA |
| 1358 | TAGTTATACCACAGCC |
| 1359 | GTAGTTATACCACAGC |
| 1360 | TGCGAATGTATGAGTG |
| 1361 | CATAGGTGTTGTGCGA |
| 1362 | GACCTTAGCACTGTCC |
| 1363 | GTCAATGCAACCTACT |
| 1364 | GCCAGGTCAATGCAAC |
| 1365 | AGCCCATCCATAGTGA |
| 1366 | ACAGCCCATCCATAGT |
| 1367 | GGTAGACTGTTGAATG |
| 1368 | CTAGGTAGACTGTTGA |
| 1369 | TCTAGGTAGACTGTTG |
| 1370 | GGTACAATATCTCTCC |
| 1371 | TCAGTCTATCAGGTAC |
| 1372 | GTAGGTTCACAGCTAG |
| 1373 | GAGTAGGTTCACAGCT |
| 1374 | GGAGTAGGTTCACAGC |
| 1375 | TTTGGCAACCCATTGC |
| 1376 | GTTGGCATACTTTGGC |
| 1377 | GAGAGTCAGTGTTACC |
| 1378 | TCGGGTTGGTGGTGAG |
| 1379 | GTCAACACTAACTTGC |
| 1380 | GGTCAACACTAACTTG |
| 1381 | ATGGAAGATCATACCC |
| 1382 | GGCACAACATACTGTC |
| 1383 | TAGGACTATGCACAAG |
| 1384 | GAACTTAGCACACTAG |
| 1385 | GGAACTTAGCACACTA |
| 1386 | TAGGAACTTAGCACAC |
| 1387 | GCAACCTGATGAGTAG |
| 1388 | TAGCAACCTGATGAGT |
| 1389 | GTAGCAACCTGATGAG |
| 1390 | TTAAGCAGTAGCAACC |
| 1391 | AACCTTAAGCAGTAGC |
| 1392 | GAACCTTAAGCAGTAG |
| 1393 | GGAACCTTAAGCAGTA |
| 1394 | GGGAACCTTAAGCAGT |
| 1395 | CTTCGATCTCAGGGCT |
| 1396 | GTACTTGGACTTCACC |
| 1397 | CCCAAGGTTAGAGTAC |
| 1398 | CAGTAAGGCATCGTTT |
| 1399 | CCAGTAAGGCATCGTT |
| 1400 | TCCAGTAAGGCATCGT |
| 1401 | ACCAAGAGTTGAGTAC |
| 1402 | TAGCACTACAATATCC |
| 1403 | CTGAGTTACGGTTAGC |
| 1404 | TCTGAGTTACGGTTAG |
| 1405 | ATCTGAGTTACGGTTA |
| 1406 | TATCTGAGTTACGGTT |
| 1407 | ATATCTGAGTTACGGT |
| 1408 | GTGGAGTGATTGATAC |
| 1409 | CGTGGAGTGATTGATA |
| 1410 | GCAGCTTAGATGATAC |
| 1411 | CTATGGACTTACTGGT |
| 1412 | GAACTATGGACTTACT |
| 1413 | GGAACTATGGACTTAC |
| 1414 | GGGAACTATGGACTTA |
| 1415 | CGTCAATGTGAAGATT |
| 1416 | GCGTCAATGTGAAGAT |
| 1417 | GAAACACGGATGGTTC |
| 1418 | GCACAGGTCTCATATG |
| 1419 | ATGTTAGGTTGGAGTC |
| 1420 | GTTGACACTTATCTGG |
| 1421 | GGTGTTCAAAGTTGAC |
| 1422 | TGGGTAACTATTGAGG |
| 1423 | CTGGATTACTATCAGG |
| 1424 | GCTGGATTACTATCAG |
| 1425 | CTGCTGGATTACTATC |
| 1426 | GTTGACCCTATTGATT |
| 1427 | TGTTGACCCTATTGAT |
| 1428 | CTGTTGACCCTATTGA |
| 1429 | GCTGTTGACCCTATTG |
| 1430 | GTGCTGTTGACCCTAT |
| 1431 | TGTGCTGTTGACCCTA |
| 1432 | GTGTGCTGTTGACCCT |
| 1433 | ATAGGGCTTGAATCAG |
| 1434 | TAACTGGGATAGGGCT |
| 1435 | GCTGAACCTTGTGATA |
| 1436 | GTGCTGAACCTTGTGA |
| 1437 | GACTGTTGAACTGGCC |
| 1438 | GGACTGTTGAACTGGC |
| 1439 | GGGACTGTTGAACTGG |
| 1440 | TAGGGACTGTTGAACT |
| 1441 | GTAGGGACTGTTGAAC |
| 1442 | GCAGTTAGTAGTATCT |
| 1443 | AGCAGTTAGTAGTATC |
| 1444 | GGAAACTAGCCTTAGG |
| 1445 | GTTGGTATAGAGCATG |
| 1446 | AGTTGGTATAGAGCAT |
| 1447 | TAAGTTGGTATAGAGC |
| 1448 | GGTTATCAGTAGGCTT |
| 1449 | GGGTTATCAGTAGGCT |
| 1450 | TGGGTTATCAGTAGGC |
| 1451 | CGTTCAAAGGGCATGG |
| 1452 | GCTAGGACACAGACGC |
| 1453 | GTAGACCACATCACTT |
| 1454 | ACGGTCCTCCCAAATC |
| 1455 | CACGGTCCTCCCAAAT |
| 1456 | GTCCACCCTTAGAAGA |
| 1457 | TGTCCACCCTTAGAAG |
| 1458 | GATGTCCACCCTTAGA |
| 1459 | TGATGTCCACCCTTAG |
| 1460 | GCTCATAATGATGTCC |
| 1461 | GTATTAGATGGAATCC |
| 1462 | CTTAGTTGGTAGTATC |
| 1463 | GGTCCTAGTAAACACT |
| 1464 | GCTCATAGAGGTCTTA |
| 1465 | GTCAGTGCAAGTAAGT |
| 1466 | GGCAGTTCAGTCCTCC |
| 1467 | CTTGGCAGTTCAGTCC |
| 1468 | CTAACCTATCCTATGG |
| 1469 | GCTAACCTATCCTATG |
| 1470 | ACAAGCTAACCTATCC |
| 1471 | TACAAGCTAACCTATC |
| 1472 | CGAAGACTTGGTTCAT |
| 1473 | GCTTTGTGGTTTGCGA |
| 1474 | ATTAAAGTAAGCCCGG |
| 1475 | CATTAAAGTAAGCCCG |
| 1476 | GCATTAAAGTAAGCCC |
| 1477 | AGTATTGAACTAGCCT |
| 1478 | CAAGTATTGAACTAGC |
| 1479 | CAGTACAACCTAGCCC |
| 1480 | CCAGTACAACCTAGCC |
| 1481 | CCCAGTACAACCTAGC |
| 1482 | TAGTGGTGGTTTACGG |
| 1483 | ATAGTGGTGGTTTACG |
| 1484 | TATAGTGGTGGTTTAC |
| 1485 | GTGGTAGATTGGATTC |
| 1486 | GTGTGGTAGATTGGAT |
| 1487 | TTGTGTGGTAGATTGG |
| 1488 | CTTGGGTAAGGTTCAA |
| 1489 | CTCTTGGGTAAGGTTC |
| 1490 | AGGACTAATACACGTA |
| 1491 | GGTCCCAATACTATCC |
| 1492 | GTTACCTCCAAGCAAT |
| 1493 | GTTACACCAGAGCAGC |
| 1494 | GATGAGTGTCTAAACC |
| 1495 | GGAGTAGGGTCATTTA |
| 1496 | CTAGCTTTATGGAACC |
| 1497 | TTCTCACAGCGAACCC |
| 1498 | TTTCTCACAGCGAACC |
| 1499 | GCCTCCCATCAATGAT |
| 1500 | GCATCATACGACATTC |
| 1501 | GCAGATTGTAGGGTAC |
| 1502 | GTTGCTACACTTGCCA |
| 1503 | AGTTGCTACACTTGCC |
| 1504 | CAGTTGCTACACTTGC |
| 1505 | CCAGTTGCTACACTTG |
| 1506 | AGTCACCCTCTAATTG |
| 1507 | GTAAGTCACCCTCTAA |
| 1508 | GCAGTAAGTCACCCTC |
| 1509 | TTGCAAGATTCCGCTT |
| 1510 | ATTGCAAGATTCCGCT |
| 1511 | TATTGCAAGATTCCGC |
| 1512 | TGTATTAGTGGCTTGC |
| 1513 | CTATGTATTAGTGGCT |
| 1514 | GGTATAGCAGACACTC |
| 1515 | GGGTCTCCATCATTGT |
| 1516 | GCATTACATGGGTCTC |
| 1517 | GCTAATGGGTTTGTGC |
| 1518 | CGGAATAAAGTTGCTA |
| 1519 | CTTAGTTAGAAGGTGT |
| 1520 | CAACTTAGTTAGAAGG |
| 1521 | GGTGTCCCAAGTAAAC |
| 1522 | CTCATTGACTTGCAAC |
| 1523 | TGGAGTTGTACTGAAC |
| 1524 | GGACTGGAGTTGTACT |
| 1525 | ATAATTCAACGCAGTG |
| 1526 | CAGAATAATTCAACGC |
| 1527 | GATAGTTTGCCTACTC |
| 1528 | GGATAGTTTGCCTACT |
| 1529 | AATAGGATAGTTTGCC |
| 1530 | GAATAGGATAGTTTGC |
| 1531 | GTTCTAAGGTGTCAAG |
| 1532 | GAAGTTCTAAGGTGTC |
| 1533 | TGGACAGTTACAGACC |
| 1534 | CTCGTATTGAAAGGTA |
| 1535 | TCTCGTATTGAAAGGT |
| 1536 | ATCTCGTATTGAAAGG |
| 1537 | ACTGCTCCTGATATAG |
| 1538 | CCCTTGCAGATGATAC |
| 1539 | TTTAGTAAGGATCACC |
| 1540 | GTTAGGCATATCATAC |
| 1541 | GGTGCTAATAACTCCT |
| 1542 | GTGGGTGCTAATAACT |
| 1543 | GTGTTAGTCATGTACA |
| 1544 | AGTGTTAGTCATGTAC |
| 1545 | GAAGTTTATGTGATCC |
| 1546 | GGTTAGGGAATCATTC |
| 1547 | ATTATTGCCCTACTAG |
| 1548 | GTGTATCCACTCAACC |
| 1549 | AAATATTCCCTCCGGC |
| 1550 | GTCCACCAGTAGATTT |
| 1551 | GCCAGTGTTGTCTACC |
| 1552 | GGGAGTATGAGCTGTT |
| 1553 | GGCTTGTAATACCTAC |
| 1554 | CGGCTTGTAATACCTA |
| 1555 | CCGGCTTGTAATACCT |
| 1556 | GGTCTCTACACTAGTT |
| 1557 | GACCTAAGCCAGATTA |
| 1558 | GATTAGACCAGACCCT |
| 1559 | AGATTAGACCAGACCC |
| 1560 | AGGATACTTAACCCTC |
| 1561 | GAGGATACTTAACCCT |
| 1562 | GGAGGATACTTAACCC |
| 1563 | GCATCCTACATCAACC |
| 1564 | GGCATCCTACATCAAC |
| 1565 | ACGTTGCCTCTGTGTG |
| 1566 | CTGTATTGGATAAACC |
| 1567 | GGCTATCACACTCAGA |
| 1568 | GTAAGGCACCCATGAG |
| 1569 | AGTAAGGCACCCATGA |
| 1570 | CAGTAAGGCACCCATG |
| 1571 | GACCGCTCAGTAAGGC |
| 1572 | GGACCGCTCAGTAAGG |
| 1573 | GTGGAACCTTTAACTG |
| 1574 | GTGCAAGTAGATTACA |
| 1575 | TGTGCAAGTAGATTAC |
| 1576 | GCCGAGTCTTCATGTC |
| 1577 | TGCAGTAGACTAGGGT |
| 1578 | TGTGCAGTAGACTAGG |
| 1579 | GTTGACATTGCTTGCC |
| 1580 | GTCATTACTGGATTAG |
| 1581 | GGTACAGTTTGGCTTA |
| 1582 | GTGGGTAGTATGTCAT |
| 1583 | GTCCATATGTAGAGTA |
| 1584 | CAATGTGTGTTGCGGG |
| 1585 | TCAATGTGTGTTGCGG |
| 1586 | GTCAATGTGTGTTGCG |
| 1587 | GGTCAATGTGTGTTGC |

TABLE 8
ASO sequences targeting human
ENSG00000230838 (Chr2)

| SEQ | |
|---|---|
| ID | |
| NO. | Sequence |
| 1588 | TCTTACGTGTCTGAAG |
| 1589 | CTCTTACGTGTCTGAA |
| 1590 | ACTCTTACGTGTCTGA |
| 1591 | TATGACTCTTACGTGT |
| 1592 | ATATGACTCTTACGTG |
| 1593 | TACCTTGAGTGACCCA |
| 1594 | TTACCTTGAGTGACCC |
| 1595 | TCGGTAATGCTATGCA |
| 1596 | ATCGGTAATGCTATGC |
| 1597 | CGCTCTTGGTTGACAT |
| 1598 | TCGCTCTTGGTTGACA |
| 1599 | GTCATTGTATAGCCAG |
| 1600 | CTGTCATTGTATAGCC |
| 1601 | CTTGAGTACCTTACAC |
| 1602 | GACCCTAGCACTTGAG |
| 1603 | AGACCCTAGCACTTGA |
| 1604 | GATTGGGCTTGGACAC |
| 1605 | TGATTGGGCTTGGACA |
| 1606 | TTGATTGGGCTTGGAC |
| 1607 | CGATAGTTCAGTCTTC |
| 1608 | GCGATAGTTCAGTCTT |
| 1609 | TGCGATAGTTCAGTCT |
| 1610 | GTGCGATAGTTCAGTC |
| 1611 | TGTGCGATAGTTCAGT |
| 1612 | ATGTGCGATAGTTCAG |
| 1613 | AATGTGCGATAGTTCA |
| 1614 | TAATGTGCGATAGTTC |
| 1615 | GTAATGTGCGATAGTT |
| 1616 | GGTAATGTGCGATAGT |
| 1617 | TAGGTAATGTGCGATA |
| 1618 | TTAGGTAATGTGCGAT |
| 1619 | CTTAGGTAATGTGCGA |
| 1620 | TCTTAGGTAATGTGCG |
| 1621 | ATCTGTCACACACCGG |
| 1622 | AATCTGTCACACACCG |
| 1623 | CTGGTGGTTGGTACAA |
| 1624 | TGAAGTAGCCATAGCC |
| 1625 | CTATTATACCAGAGGC |
| 1626 | CGACATTTCAGCACTT |
| 1627 | ACGACATTTCAGCACT |
| 1628 | TACGACATTTCAGCAC |
| 1629 | GTACGACATTTCAGCA |
| 1630 | AGTACGACATTTCAGC |
| 1631 | CAGTACGACATTTCAG |
| 1632 | TCAGTACGACATTTCA |
| 1633 | TGAAGTGTCAGTACGA |
| 1634 | GGAGTCTAGGTAGCTA |
| 1635 | GAAGGCACCCTACACT |
| 1636 | GTGAAGGCACCCTACA |
| 1637 | GAAGATTACCTAGCAG |
| 1638 | GGCATAGAAGATTACC |
| 1639 | CCATTAGGGCATAGAA |
| 1640 | GACGTGGTTCTCCTAT |
| 1641 | CCAGAACTATTACGAA |
| 1642 | ACCAGAACTATTACGA |
| 1643 | GTACCAGAACTATTAC |
| 1644 | GCAGTACCAGAACTAT |
| 1645 | GGTCTCTTTACTGGAC |
| 1646 | CCATCAGCAGTACCGT |
| 1647 | CATTGTGTTGGGACGT |
| 1648 | GCATTGTGTTGGGACG |
| 1649 | GACCTTTAGCCACTCA |
| 1650 | CCTTGACCATATTGCA |
| 1651 | GACCCATTATAGTTTC |
| 1652 | CAGACCCATTATAGTT |
| 1653 | TACAGACCCATTATAG |
| 1654 | GCTATTACAGACCCAT |

TABLE 9
ASO sequences targeting human
ENSG00000244137 (Chr1)

| SEQ | |
|---|---|
| ID | |
| NO. | Sequence |
| 1655 | CAGCACGTTTCTTAGC |
| 1656 | GCAGCACGTTTCTTAG |
| 1657 | TGCAGCACGTTTCTTA |
| 1658 | TGATGTGTGTGGACCC |
| 1659 | CTATGGGTGATGTTAG |
| 1660 | GTGAACACCTCCTAGA |
| 1661 | GTCTACCTATGAACTA |
| 1662 | GCTAGAGGAATTGTAT |
| 1663 | GACCCAGTGAACCTAG |
| 1664 | GTATGTAACCTGGAGC |
| 1665 | TTTGTGAACTTTCCGG |
| 1666 | CAAGACTGCCTAGTGG |
| 1667 | ACAAGACTGCCTAGTG |
| 1668 | GACATCCTTAGCCAGC |
| 1669 | CTTCAATAGTGCAGAC |
| 1670 | GCACAGTAGAGTAATC |
| 1671 | GACGTATCCTTCCAAA |
| 1672 | TCATAAGAGAGTAGCC |
| 1673 | TGGATGAAGGCGTATT |
| 1674 | GTATTGACCATGCCTC |
| 1675 | GGTCGTATAATACTTC |
| 1676 | GGGTCGTATAATACTT |
| 1677 | AGGGTCGTATAATACT |
| 1678 | AAGGGTCGTATAATAC |
| 1679 | CTAAGGGTCGTATAAT |
| 1680 | TGCAACAGTCTATGGT |
| 1681 | GTTATGAGGATGGTTC |
| 1682 | ATCGTATGGATGGGCC |
| 1683 | GACAACATTCCATCGT |
| 1684 | GTTAACCCTGTACCCA |
| 1685 | GTGTTAACCCTGTACC |
| 1686 | GCGTAGACATATAATG |
| 1687 | CGATGCAGGTTTATAG |
| 1688 | ACGATGCAGGTTTATA |
| 1689 | GCTGATTTGTAGGTAG |
| 1690 | GGCTGATTTGTAGGTA |
| 1691 | GGGCTGATTTGTAGGT |
| 1692 | CAAGCGACCACACTGC |
| 1693 | ACAAGCGACCACACTG |
| 1694 | GACAAGCGACCACACT |
| 1695 | GGACAAGCGACCACAC |
| 1696 | GAGTAAGTGCCAACCC |
| 1697 | AGAGTAAGTGCCAACC |
| 1698 | AGATGCGGGACTATTT |
| 1699 | CAGATGCGGGACTATT |
| 1700 | CCAGATGCGGGACTAT |
| 1701 | GCCTTAGATCCATGAT |
| 1702 | GTGCCTTAGATCCATG |
| 1703 | AGTGCCTTAGATCCAT |
| 1704 | GAGTGCCTTAGATCCA |
| 1705 | TCCTCATCAATAGGTC |
| 1706 | AGGTCTATGTCAGTGC |
| 1707 | CATAACACACGGCTAG |
| 1708 | GCATAACACACGGCTA |
| 1709 | AGCATAACACACGGCT |
| 1710 | CAGCATAACACACGGC |
| 1711 | ACAGCATAACACACGG |
| 1712 | CACAGCATAACACACG |
| 1713 | GTTAGAAGCACTCCAC |
| 1714 | AGGCTTCAGGACGGAC |
| 1715 | TAGAGTTCAGCCCTCC |
| 1716 | GCAGGAGTTTCAGTCG |
| 1717 | AAGCGGTGAATGTTGC |
| 1718 | CGCAGAAGTTGTCAGA |
| 1719 | AGAGGTTCATGCGGGT |
| 1720 | TAAAGAGGTTCATGCG |
| 1721 | ACCACAGGATCGGATG |
| 1722 | GACCACAGGATCGGAT |
| 1723 | GGTGCTAATGACTCTA |
| 1724 | GGTCCCTATTCAATTG |
| 1725 | GTATAAGCCTAGCTCA |
| 1726 | CTATGCACCTATTTGG |
| 1727 | AGTTCTGTTCGATGTC |
| 1728 | AAGTTCTGTTCGATGT |
| 1729 | AAAGTTCTGTTCGATG |
| 1730 | GTATGGGATTCAGGTG |
| 1731 | ATGGAGTATGGGATTC |
| 1732 | GTAGACATTGACCTTC |
| 1733 | GGTCTAACACAATGGC |
| 1734 | GAACGGATGCCAGCAC |
| 1735 | CCTTAGAAGCAGAACG |
| 1736 | CGATAGTGAGTTTGCA |
| 1737 | ACGATAGTGAGTTTGC |
| 1738 | TCATAGGTGATGGGAC |
| 1739 | GTTCAAGGTCTCGCAC |
| 1740 | GGTTCAAGGTCTCGCA |
| 1741 | AGGTTCAAGGTCTCGC |
| 1742 | ACTTCTACTAGGGCTA |
| 1743 | GTGCTATAACCAAGAC |
| 1744 | TTCGAGAGAGGAATTC |
| 1745 | TTGAACGTCATAGTGA |
| 1746 | TTTGAACGTCATAGTG |
| 1747 | GCTATCCTCGTGATAC |
| 1748 | GCCCTTGTTAAAGTAC |
| 1749 | GGTGCTGCTATTCTAC |
| 1750 | TTATGCGTCTTTGGAG |
| 1751 | TTTATGCGTCTTTGGA |
| 1752 | GTTTATGCGTCTTTGG |
| 1753 | CTACCACTCAATAGGC |
| 1754 | TATGTTATGGATGCCC |
| 1755 | GTTATGTTATGGATGC |
| 1756 | GGTTTAGCTCTCAGAC |
| 1757 | GCATTGGTTTAGCTCT |
| 1758 | GACCCAAGTTATGCCA |
| 1759 | AGACCCAAGTTATGCC |
| 1760 | GAGACCCAAGTTATGC |
| 1761 | GACATCTCTTATGGGT |
| 1762 | ATCCATGCAGATTGAC |
| 1763 | ACCCTGTATGAGATCC |
| 1764 | TACCCTGTATGAGATC |
| 1765 | GTACCCTGTATGAGAT |
| 1766 | AGTACCCTGTATGAGA |
| 1767 | TATGAAGTCTAGTGGG |
| 1768 | TTAGATTGGTGGCATG |
| 1769 | GAATTAGATTGGTGGC |
| 1770 | TTTCGTTTAGTGAGAG |
| 1771 | AGCTAGATAAGTCTCC |
| 1772 | GACCCTATTACCAAGA |
| 1773 | GCAAGGCACATATCCC |
| 1774 | GAGTACATCCTGATTC |
| 1775 | GTCCATGAATTGTGTC |
| 1776 | TGACAATCGGCTATGC |
| 1777 | CTGACAATCGGCTATG |
| 1778 | ATGTGCTGACAATCGG |
| 1779 | AATGTGCTGACAATCG |
| 1780 | GGAGGAGTGTAGTTTC |
| 1781 | TCATGTTTGGTAGGAC |
| 1782 | GACTTATTGAACAGGG |
| 1783 | GGACTTATTGAACAGG |
| 1784 | TAGCAGACTAGACCCA |
| 1785 | TTAGCAGACTAGACCC |
| 1786 | TTTAGCAGACTAGACC |
| 1787 | GCCCACCATCAAGTAG |
| 1788 | ATCCTACTCAAACGGC |
| 1789 | GGTCAATCCTACTCAA |
| 1790 | AATCCTCGGTGGCAAA |
| 1791 | CAATCCTCGGTGGCAA |
| 1792 | GTCCAGTGAATGCTAT |
| 1793 | CCTCATTCACACGAAC |
| 1794 | CCCTCATTCACACGAA |
| 1795 | CTCATGCCAGTTTAGC |
| 1796 | GACTCATGCCAGTTTA |
| 1797 | AACCTCACTCCCGATG |
| 1798 | GCAACCTCACTCCCGA |
| 1799 | GGTAGTTCAGGTCTCC |
| 1800 | ACAGACACCTCGTGCC |
| 1801 | TACAGACACCTCGTGC |
| 1802 | TTACAGACACCTCGTG |
| 1803 | GTACTATGATCTCTTG |
| 1804 | GATGTACTATGATCTC |
| 1805 | GCACTAACTTCCTAGC |
| 1806 | CGGCACTAACTTCCTA |
| 1807 | TCTCACTTGGACACCG |
| 1808 | CGTGCCCAAACCTGAC |
| 1809 | ACGTGCCCAAACCTGA |
| 1810 | GGTCTACTTTAACATC |
| 1811 | GTCCTCTACAGCAAGC |
| 1812 | GCACTCCAACACTATC |
| 1813 | TAGCACTCCAACACTA |
| 1814 | CTTAGCACTCCAACAC |
| 1815 | TTACGTATGAATTCCC |
| 1816 | CTTACGTATGAATTCC |
| 1817 | GATGTACCTCCTGCAA |
| 1818 | GCTCTAGGATGTACCT |
| 1819 | AGCTCTAGGATGTACC |
| 1820 | CAGCTCTAGGATGTAC |
| 1821 | GAGTGATCCATTGCCC |
| 1822 | TGAGTGATCCATTGCC |
| 1823 | GTGAGTGATCCATTGC |
| 1824 | CGTCCATGTGAGTGAT |
| 1825 | AGAGCGTCCATGTGAG |
| 1826 | AAGAGCGTCCATGTGA |
| 1827 | CTCGGAAATTCACAGC |
| 1828 | GGAAACTCGGAAATTC |
| 1829 | GGTGCTTGAGAGATTC |
| 1830 | CCATGAGGGTTTAGGT |
| 1831 | CTCATTATGACAGTCC |
| 1832 | AGTAGTGAATAGACCC |
| 1833 | CAGTAGTGAATAGACC |
| 1834 | GGTAACACTAGCTGCA |
| 1835 | TATATCTGTGGAGGGC |
| 1836 | GTACCTATATCTGTGG |
| 1837 | GCAGTACCTATATCTG |
| 1838 | GGCAGTACCTATATCT |
| 1839 | GCTCCAACCAATCAAG |
| 1840 | TCTTGATGAGAGGTAC |
| 1841 | TAGCCTGACACACCTC |
| 1842 | TCTAGCCTGACACACC |
| 1843 | CGGTTTGTTGAAGAGT |
| 1844 | CTCGGTTTGTTGAAGA |
| 1845 | TCTCGGTTTGTTGAAG |
| 1846 | CTCTCGGTTTGTTGAA |
| 1847 | GCTCTCGGTTTGTTGA |
| 1848 | AGCTCTCGGTTTGTTG |
| 1849 | TACACCCAGATTAGAG |
| 1850 | GTTACTAAGCAGGTCT |
| 1851 | AGTTACTAAGCAGGTC |
| 1852 | GACCCAGTGAGTAGTT |
| 1853 | AGCTTGTCCATTAAGG |
| 1854 | GTAAGGCATTATCACC |
| 1855 | GGGTACAGACAACATC |
| 1856 | CGGGTACAGACAACAT |
| 1857 | GCGAGGTCACATACAA |
| 1858 | AGCGAGGTCACATACA |
| 1859 | CAGCGAGGTCACATAC |
| 1860 | CCAGCGAGGTCACATA |
| 1861 | GGCACACCTAGCTCAC |
| 1862 | ACTATTTGGAGGCGGC |
| 1863 | AACTATTTGGAGGCGG |
| 1864 | GAACTATTTGGAGGCG |
| 1865 | GTGACCTACTTTGAAC |
| 1866 | TGAAGGCACCGCATGG |
| 1867 | TTGAAGGCACCGCATG |
| 1868 | GTTGAAGGCACCGCAT |
| 1869 | CATGTGAGTAGGGTTG |
| 1870 | TGTATTAGCCAGACCC |
| 1871 | GTAGTTGTGAGTAACT |
| 1872 | TGTAGTTGTGAGTAAC |
| 1873 | ACTCTAACTTTCACGA |
| 1874 | GCCCACCTTGCATCAA |
| 1875 | GGTCAGTCACCCTATC |
| 1876 | GGACTACTCTTTACCA |
| 1877 | AGGACTACTCTTTACC |
| 1878 | CTGGTTAGTTGATAAC |
| 1879 | CGAAGAGTTGATGTTA |
| 1880 | CCGAAGAGTTGATGTT |
| 1881 | TCCGAAGAGTTGATGT |
| 1882 | CTCCGAAGAGTTGATG |
| 1883 | TTCCACGTAACAACAG |
| 1884 | TATTCCACGTAACAAC |
| 1885 | GTATTCCACGTAACAA |
| 1886 | GATTGCTCACTCAAGC |
| 1887 | GTTGGACTCTTTGGGA |
| 1888 | CATCCTCTGAATGCGT |
| 1889 | ACATCCTCTGAATGCG |
| 1890 | GCCAAAGGTATCACTC |
| 1891 | ACGAGTCCCATTCTTG |
| 1892 | TCACCACGAGTCCCAT |
| 1893 | TAATCACCACGAGTCC |
| 1894 | TTAATCACCACGAGTC |
| 1895 | ATTAATCACCACGAGT |
| 1896 | GTGTAGTTCTGGTCTG |
| 1897 | GGTGTAGTTCTGGTCT |
| 1898 | GTGGTGTAGTTCTGGT |
| 1899 | GCTTGAACTTATATGG |
| 1900 | GGCTTGAACTTATATG |
| 1901 | AACTCGTTTAGCAGCT |
| 1902 | AAACTCGTTTAGCAGC |
| 1903 | TTCCACCGCAGCACAA |
| 1904 | ATTCCACCGCAGCACA |
| 1905 | ATGCTAATCCCTTAGG |
| 1906 | GTGACAATGCTAATCC |
| 1907 | GTCATGCCTACAGTGT |
| 1908 | TTATTAAGGGTCATGC |
| 1909 | TTAGATTAGCCTTGGG |
| 1910 | CTTAGATTAGCCTTGG |
| 1911 | GACTGCTATGACACTA |
| 1912 | GGACTGCTATGACACT |
| 1913 | TACTGAAACGGGATCT |
| 1914 | TTTACTGAAACGGGAT |
| 1915 | CATCAAGGTCAGTTGG |
| 1916 | GGGCATAACTCTTGGG |
| 1917 | CGAGAACTTCTATGAG |
| 1918 | CCGAGAACTTCTATGA |
| 1919 | CTTCAGGCTTAGTTAG |
| 1920 | GCCCTAGTCTTACCAG |
| 1921 | AGCCCTAGTCTTACCA |
| 1922 | ATAGCCCTAGTCTTAC |
| 1923 | TTTAATAGCCCTAGTC |
| 1924 | CTTTAATAGCCCTAGT |
| 1925 | TTCTGATTACAAGCGC |
| 1926 | ATTCTGATTACAAGCG |
| 1927 | CACTAACCAATAGCCC |
| 1928 | CCACTAACCAATAGCC |
| 1929 | TCAACCAACGAAATAG |
| 1930 | CTTGGGTTATCATACA |
| 1931 | ACTTGGGTTATCATAC |
| 1932 | CCATGCAGATTAGAGT |
| 1933 | TGGATTGAGAGGTTAC |
| 1934 | GTTGGATTGAGAGGTT |
| 1935 | GCTGAACAATCGAGTG |
| 1936 | AGCTGAACAATCGAGT |
| 1937 | TGAGCTGAACAATCGA |
| 1938 | GCTATGCTCTAGTTGC |
| 1939 | GCCTAGATGTGCCCAT |
| 1940 | TAGCCTAGATGTGCCC |
| 1941 | GTAGCCTAGATGTGCC |
| 1942 | CGTAGCCTAGATGTGC |
| 1943 | TCGTAGCCTAGATGTG |
| 1944 | CTGTCGTAGCCTAGAT |
| 1945 | CCTGTCGTAGCCTAGA |
| 1946 | TCCTGTCGTAGCCTAG |
| 1947 | TAGTGAGCATCTAAGG |
| 1948 | ACTAGTCTTTGTAGTG |
| 1949 | AGTCTCTACACTAGTC |
| 1950 | TAGCTTGGTCAGAGGG |
| 1951 | GTAGCTTGGTCAGAGG |
| 1952 | GGTAGCTTGGTCAGAG |
| 1953 | GGGTAGCTTGGTCAGA |
| 1954 | TATGGGTAGCTTGGTC |
| 1955 | CTATGGGTAGCTTGGT |
| 1956 | TTGTAACTTGGTGGAC |
| 1957 | CTTGTAACTTGGTGGA |
| 1958 | GCTTGTAACTTGGTGG |
| 1959 | AGCTTGTAACTTGGTG |
| 1960 | CAGCTTGTAACTTGGT |
| 1961 | GCTTTGATGGGTTGAC |
| 1962 | ACTTGTGAGTCCTGTC |
| 1963 | GTCTACTTGTGAGTCC |
| 1964 | GATTACTGAGTCCACC |
| 1965 | CCTAACCCAACTGTGC |
| 1966 | ACCTAACCCAACTGTG |
| 1967 | GAGTGACCCTGTTGAT |
| 1968 | CTCATTAACTCCCGAG |
| 1969 | CTTGGTTTGATGGCGC |
| 1970 | TCTTGGTTTGATGGCG |
| 1971 | GCCCTGTCACCAATAG |
| 1972 | GTCCTAAACCATGCTA |
| 1973 | CCTTACGCTTAATCAA |
| 1974 | TTATGATAGACGGGCA |
| 1975 | TTTATGATAGACGGGC |
| 1976 | GCATGTCCTATAACAG |
| 1977 | GTTCAGTAGGTCAGTC |
| 1978 | GGAAGTTCAGTAGGTC |
| 1979 | GGTCATGTAGAGTGCT |
| 1980 | TGGTCATGTAGAGTGC |
| 1981 | CTGGTCATGTAGAGTG |
| 1982 | GATCTGTTGCCACCCA |
| 1983 | GTGATCTGTTGCCACC |
| 1984 | TTAGTGGATGGTTGAT |
| 1985 | CTTAGTGGATGGTTGA |
| 1986 | CCTTAGTGGATGGTTG |
| 1987 | ACCTTAGTGGATGGTT |
| 1988 | TACCTTAGTGGATGGT |
| 1989 | TTACCTTAGTGGATGG |
| 1990 | TACATTCAGGTGGTCC |
| 1991 | GACTGATAGACCATAC |
| 1992 | TTGACTGATAGACCAT |
| 1993 | GATTGACTGATAGACC |
| 1994 | ACCTAGACTGATGTTA |
| 1995 | GCAACTGAGCTATCCT |
| 1996 | GGTCTATTGGTTAAGA |
| 1997 | AGCTTGTGTGGACCCT |
| 1998 | GACTTAATCCAACTGG |
| 1999 | GCTAACCATTATACAC |
| 2000 | AGCCCTTACTGCTAAC |
| 2001 | GAGCCCTTACTGCTAA |
| 2002 | AGAGCCCTTACTGCTA |
| 2003 | TGGTAGACAGGCAATC |
| 2004 | ATGTTTAGTCCAAGGC |
| 2005 | GTATGTTTAGTCCAAG |
| 2006 | GAGTATGTTTAGTCCA |
| 2007 | GGGAGTATGTTTAGTC |
| 2008 | GATTGGCTGTTACCTC |
| 2009 | AGGATTGGCTGTTACC |
| 2010 | CGCTGCCACAGGATTG |
| 2011 | TTGGTCCTGATAATCC |
| 2012 | CTTGGTCCTGATAATC |
| 2013 | GAACCCTACTGCTGAG |
| 2014 | GAGTAGCATTGGCAGT |
| 2015 | GCAATTGAGACACCTC |
| 2016 | GCAGCAGTCTAGGTTC |
| 2017 | GCCCATGTGAACAACC |
| 2018 | TCTAGCCCATGTGAAC |
| 2019 | GTATCGGGTGAGGTCT |
| 2020 | GTGATAGGCTCAACCT |
| 2021 | ATCTCAGCAGCCCGTG |
| 2022 | CTCAGCATTACACGGT |
| 2023 | ACTCAGCATTACACGG |
| 2024 | GACTCAGCATTACACG |
| 2025 | GGACTCAGCATTACAC |
| 2026 | GTAATCTGTACTCTAC |
| 2027 | GAGTAGAGCACTTAGC |
| 2028 | CTGCGTTACCATGAGT |
| 2029 | GCTGCGTTACCATGAG |
| 2030 | AGCTGCGTTACCATGA |
| 2031 | GCCCACGACCATTAGG |
| 2032 | GATTAGTAGGTCATGC |
| 2033 | GGATTAGTAGGTCATG |
| 2034 | AGGATTAGTAGGTCAT |
| 2035 | CAGGATTAGTAGGTCA |
| 2036 | ACAGGATTAGTAGGTC |
| 2037 | GGGTTGCACTTTACAG |
| 2038 | GCCCTATCGGAGTTGG |
| 2039 | AGGTAATTGATCCCAC |
| 2040 | CCAGGTAATTGATCCC |
| 2041 | TTCCGAAGTGGTAAGG |
| 2042 | TTTCCGAAGTGGTAAG |
| 2043 | GTTTCCGAAGTGGTAA |
| 2044 | TGTTTCCGAAGTGGTA |
| 2045 | GTGTTTCCGAAGTGGT |
| 2046 | GGTGTTTCCGAAGTGG |
| 2047 | GACTCATCATAACTAC |
| 2048 | GGACTCATCATAACTA |
| 2049 | CCTACAAGTGAGATCC |
| 2050 | GTCCTACAAGTGAGAT |
| 2051 | GGTCCTACAAGTGAGA |
| 2052 | TGGTCCTACAAGTGAG |
| 2053 | AGTGGGTCTTGTCAGC |
| 2054 | GAGTGGGTCTTGTCAG |
| 2055 | GTGCTGAATCACTATG |
| 2056 | AACCATATGCAGGATC |
| 2057 | CGGAATACAAGCAATC |
| 2058 | GGGTATGGGTTCAACA |
| 2059 | AGGGTATGGGTTCAAC |

In some instances, the modulator described herein comprises one or more sugar-modified nucleotide. In some specific instances, the sugar-modified nucleotide is a 2′-fluoro modified nucleotide. In some specific instances, the sugar-modified nucleotide is a 2′-alkoxy modified nucleotide (e.g., 2′-methoxy modified nucleotide). In some specific instances, the sugar-modified nucleotide is a 2′-amino modified nucleotide. In some specific instances, the sugar-modified nucleotide is a 2′-azido modified nucleotide.

In some instances, the modulator described herein comprises one or more backbone-modified nucleotide. In some specific instances, the modified backbone is a methylphosphonate.

In some specific instances, the modified backbone is phosphorothioate. In some specific instances, the modified backbone is a guanidinopropyl phosphoramidate. In some specific instances, the modified backbone is a mesyl-phosphoramidate (MsPA) linkages.

In some specific instances, the modified backbone is phosphorothioate, and the phosphorothioate is a stereochemically enriched phosphorothioate. In certain instances, the strand contains at least one stereochemically enriched phosphorothioate. In some instances, the strand comprises at least 1, 2, 3 stereochemically enriched phosphorothioates. In some instances, the strand comprises only 1, 2, 3, or 4 stereochemically enriched phosphorothioates.

In some instances, the modulator described herein comprises one or more purine modification. In some specific instances, the purine modification described herein is 2,6-diaminopurine. In some specific instances, the purine modification described herein is 3-deaza-adenine. In some specific instances, the purine modification described herein is 7-deaza-guanine. In some specific instances, the purine modification described herein is 8-azido-adenine.

In some instances, the modulator described herein comprises one or more pyrimidine modification. In some specific instances, the pyrimidine modification described herein is 2-thiothymidine. In some specific instances, the pyrimidine modification described herein is 5-carboxamide-uracil. In some specific instances, the pyrimidine modification described herein is 5-methyl-cytosine. In some specific instances, the pyrimidine modification described herein is 5-ethynyl uracil.

In some instances, the modulator described herein comprises an abasic substitution. In those cases where a hybridized polynucleotide construct is contemplated for use as siRNA, a reduction of miRNA-like off-target effects is desirable. The inclusion of one or more (e.g., one or two) abasic substitutions in the hybridized polynucleotide constructs may reduce or even eliminate miRNA-like off-target effects, as the abasic substitutions lack nucleobases that are capable of engaging in base-pairing interactions and alleviate steric hindrance. Thus, the modulator disclosed herein may include one or more (e.g., one or two) abasic substitutions. In specific instances, abasic substitution is at the 5th nucleotide from the 5′ end of the antisense strand described herein. The modulator described herein may contain a strand including a seed region including a hypoxanthine nucleobase-containing nucleoside (e.g., inosine).

In some instances, the modulator described herein comprises one or more type of modifications as described above. Accordingly, in some instances, about 10% of the nucleotides from the modulator described herein are modified with one or more type of modifications as described above. In other instances, about 20% of the nucleotides from the modulator described herein are modified with one or more type of modifications as described above. In other instances, about 30% of the nucleotides from the modulator described herein are modified with one or more type of modifications as described above. In other instances, about 40% of the nucleotides from the modulator described herein are modified with one or more type of modifications as described above. In other instances, about 50% of the nucleotides from the modulator described herein are modified with one or more type of modifications as described above. In other instances, about 60% of the nucleotides from the modulator described herein are modified with one or more type of modifications as described above. In other instances, about 70% of the nucleotides from the modulator described herein are modified with one or more type of modifications as described above. In other instances, about 80% of the nucleotides from the modulator described herein are modified with one or more type of modifications as described above. In other instances, about 90% of the nucleotides from the modulator described herein are modified with one or more type of modifications as described above. In other instances, 100% of the nucleotides from the modulator described herein are modified with one or more type of modifications as described above.

In some instances, the one or more types of modifications described herein occurs at different positions within the modulator described herein. In specific instances, the one or more types of modifications described herein occurs in the seed region within the modulator described herein. In specific instances, the one or more types of modifications described herein occurs at 3′ terminal of the modulator described herein. In specific instances, the one or more types of modifications described herein occurs at 5′ terminal of the modulator described herein. In specific instances, the one or more types of modifications described herein occurs dispersedly within the modulator described herein. In specific instances, the one or more types of modifications described herein occurs in clusters within the modulator described herein.

In some instances, the ASO is a gapmer comprising a central region of consecutive DNA nucleotides flanked by a 5′-wing region and 3′-wing region, wherein at least one of 5′-wing region and 3′-wing region comprises a nucleic acid analogue, wherein the nucleic acid analogue comprises one or more ribose modifications, one or more backbone modifications, one or more nucleobase modifications, or a combination thereof.

In some instances, the one or more ribose modifications disclosed herein include locked nucleic acid (LNA), tricyclo-DNA, 2′-fluoro, 2′-O-methyl, 2′-methoxyethyl (2′-MOE), 2′-cyclic ethyl (cET), unlocked nucleic acid (UNA), conformationally restricted nucleoside (CRN), or any combination thereof. In some instances, the one or more backbone modifications comprise phosphorothioate, methylphosphonate, guanidinopropyl phosphoramidate, or any combination thereof. In some instances, the one or more nucleobases comprise purine modifications (e.g., 2,6-diaminopurin, 3-deaza-adenine, 7-deaza-guanine, 8-zaido-adenine, or any combination thereof). In some instances, the one or more nucleobases comprise pyrimidine modifications (e.g., 2-thiothymidine, 5-carboxamide-uracil, 5-methyl-cytosine, 5-ethynyl-uracil, or any combination thereof).

In some instances, the nucleic acid analogue comprises an LNA. In some instances, the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA. In some instances, the LNA comprises a beta-D-oxy LNA. In some instances, the 5′-wing region comprises at least two LNAs. In some instances, the 5′-wing region comprises three consecutive LNAs. In some instances, the 3′-wing region comprises at least one LNA. In some instances, the 3′-wing region comprises two consecutive LNAs. In some instances, the nucleic acid molecule disclosed herein comprises one or more phosphorothioate internucleotide linkages. In some instances, each internucleotide linkage in the nucleic acid molecule is a phosphorothioate backbone.

In some instances, the modulator is a gapmer, and one or more assays are utilized to assess the efficiency of the gapmer. Accordingly, in some instances, the efficiency of the modulator is assessed by the expression (e.g., transcript expression) of the lncRNA disclosed herein. In some instances, the efficiency of the modulator is assessed by the features of the lncRNA disclosed herein. In some instances, the efficiency of the modulator is assessed by the binding of an RNA-binding protein to the lncRNA disclosed herein. In some instances, the efficiency of the modulator is assessed by the modification and/or functional features of the lncRNA disclosed herein. In some instances, the efficiency of the modulator is assessed by structural features of the lncRNA disclosed herein.

In some instances, the modulator is a mixmer, and one or more assays are utilized to assess the efficiency of the mixmer. Accordingly, in some instances, the efficiency of the modulator is assessed by the expression of the lncRNA disclosed herein. In some instances, the efficiency of the modulator is assessed by the features of the lncRNA disclosed herein. In some instances, the efficiency of the modulator is assessed by the binding of an RNA-binding protein to the lncRNA disclosed herein. In some instances, the efficiency of the modulator is assessed by the modification and/or functional features of the lncRNA disclosed herein. In some instances, the efficiency of the modulator is assessed by structural features of the lncRNA disclosed herein.

In some instances, the modulator is an siRNA which targets a cytoplasmic target, and one or more assays are utilized to assess the efficiency of the modulator. Accordingly, in some instances, the efficiency of the modulator is assessed by the expression of the lncRNA disclosed herein. In some instances, the efficiency of the modulator is assessed by the features of the lncRNA disclosed herein. In some instances, the efficiency of the modulator is assessed by the binding of an RNA-binding protein to the lncRNA disclosed herein. In some instances, the efficiency of the modulator is assessed by the modification and/or functional features of the lncRNA disclosed herein. In some instances, the efficiency of the modulator is assessed by structural features of the lncRNA disclosed herein.

In some instances, the modulator disclosed herein suppresses CAF activities, which may include, but not limited to involvement in regulating ECM structure organization and/or GTPase activity. In some instances, the modulator disclosed herein modulates extracellular matrix organization. In some instances, the modulator disclosed herein modulates extracellular structure organization. In some instances, the modulator disclosed herein modulates external encapsulating structure organization. In some instances, the modulator disclosed herein modulates the expression and/or components of ECM. Non-limiting examples of components of ECM include collagen, elastin, reticulin, associated-microfibrils, fibronectin, laminin, proteoglycan, proteoglycan polymer, glycoconjugate, glycosaminoglycan, or variants thereof. In some instances, the modulator disclosed herein modulates GTPase activity. In some instances, the modulator disclosed herein modulates activation of GTPase activity. Non-limiting examples of GTPase activity include signal transduction in response to activation of cell surface markers (e.g., transmembrane receptors), protein biosynthesis, regulation of cell differentiation, regulation of cell proliferation, regulation of cell division, regulation of cell movement, translocation of proteins, transportation of vesicles within cell or variations thereof. In some instances, the modulator disclosed herein modulates GTPase expression. In some instances, the modulator disclosed herein modulates GTP expression. In some instances, the modulator disclosed herein modulates the ability of GTPase to bind to GTP. In some instances, the modulator disclosed herein regulates genes involved in ECM structure organization, and/or GTPase activity. In some instances, the modulator disclosed herein reduces expression or activity of genes involved in ECM structure organization, and/or GTPase activity. In some instances, the modulator disclosed herein suppresses phenotype development of CAF. In some instances, the modulator disclosed herein induces or facilitates transition of CAFs to fibroblasts. In some instances, the modulator disclosed herein induces or facilitates transition of myCAFs to fibroblasts. In some instances, the modulator disclosed herein suppresses the transition of fibroblasts to CAFs. In some instances, the modulator disclosed herein reverses the transition of fibroblasts to CAFs. In some instances, the modulator disclosed herein suppress metastasis formation and/or drug resistance. In some instances, the modulator disclosed herein reduces expression or activity of one or more markers of myCAFs. In some instances, the modulator disclosed herein reduces expression or activity of one or more genes in listed in Table 1 or Table 13 in CAFs compared to a CAFs without modulator. In some instances, the modulator disclosed herein suppresses expression of one or more genes listed in Table 1 or Table 13 in CAFs with an activated TGFβ pathway compared to CAFs without modulator. In some instances, the modulator disclosed herein suppresses expression of one or more genes listed in Table 1 or Table 13 in HDFs treated with TGFβ with or without starvation compared to HDFs without modulator. In some instances, the modulator disclosed herein reduces expression or activity of one or more ECM-modulatory gene in CAFs when compared to a CAFs without modulator. Non-limiting examples of ECM-modulatory genes are LRRC15 (NM 001135057), MMP11 (NM_005940), COL11A1 (NM 080629), C1QTNF3 (NM 030945), CTHRC1 (NM_138455), COL12A1 (NM 004370), COL10A1 (NM 00493), COL5A2 (NM_000393), THBS2 (NM 003247), AEBP1 (NM_001129), ITGA11 (NM_001004439), PDPN (NM 006474), FAP (NM 004460), COL8A1 (NM_001850), COL1A1 (NM_000088), COL1A2 (NM 000089), FN1 (NM 212482), or POSTN (NM_006475). In some instances, higher expression of ECM-modulatory gene expression is associated with poor response to checkpoint blockade treatment, standard-of-care chemotherapy treatments of cancers, or a combination thereof. In some instances, higher expression of ECM-modulatory gene expression is associated with poor response to checkpoint blockade treatment of cancers. In some instances, the RNA expression level of a marker of myCAF is at least about 10%, at least about 20%, at least about 30%, at least about 40%, at least about 50%, at least about 60%, at least about 70, at least about 80%, or at least about 90% lower than in a myCAF with a modulator disclosed herein compared to a myCAF without a modulator. In some instances, the RNA expression level of a marker of myCAF is at least about 1.5 folds, at least about 2 folds, at least about 3 folds, at least about 4 folds, at least about 5 folds, at least about 6 folds, at least about 7 folds, at least about 8 folds, at least about 9 folds, or at least about 10 folds lower in a myCAF with a modulator disclosed herein compared to a myCAF without a modulator.

### Pharmaceutical Compositions

Further provided herein are pharmaceutical compositions comprising the modulator disclosed herein and a pharmaceutically acceptable salt, excipient, or derivative thereof.

The suitable pharmaceutically acceptable salts or derivative thereof include but are not limited to (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, and the like; and (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like.

A pharmaceutical composition described herein can be prepared to include the modulator disclosed herein, into a form suitable for administration to a subject using carriers, excipients, and vehicles. In some instances, excipients include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases.

Other pharmaceutically acceptable vehicles include aqueous solutions, non-toxic excipients, including salts, preservatives, buffers and the like, as described, for instance, in Remington: The Science and Practice of Pharmacy, 21st Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

The pharmaceutical compositions described herein may be administered in combination with one or more therapeutic agents. The pharmaceutical compositions described herein may be administered in combination with one or more therapeutic agents that mediates tumor fibrosis.

The pharmaceutical compositions described herein may be administered in combination with one or more anti-cancer drug. The pharmaceutical compositions described herein may be administered in combination with one or more anti-tumor drug.

The pharmaceutical compositions described herein may be administered locally or systemically. The therapeutically effective amounts will vary according to factors, such as the degree of infection in a subject, the age, sex, health conditions, and weight of the individual.

Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition can be sterile and fluid to the extent that easy syringability exists. The composition can be stable under the conditions of manufacture and storage and preserved against the contaminating action of microorganisms, such as bacteria and fungi. The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of certain particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in an appropriate solvent with one or a combination of ingredients enumerated above followed by filtered sterilization. Generally, dispersions are prepared by incorporating the pharmaceutical composition into a sterile vehicle that contains a basic dispersion medium and the other ingredients from those enumerated above.

It is advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the pharmaceutical vehicle. The specification for the dosage unit forms are related to the characteristics of the pharmaceutical composition and the particular therapeutic effect to be achieve. The principal pharmaceutical composition is compounded for convenient and effective administration in effective amounts with a suitable pharmaceutically acceptable vehicle in an acceptable dosage unit. In the case of compositions containing supplementary active ingredients, the dosages are determined by reference to the usual dose and manner of administration of the ingredients.

The pharmaceutical composition can be orally administered, for example, in a carrier, e.g., in an enteric-coated unit dosage form. The pharmaceutical composition and other ingredients can also be enclosed in a hard or soft-shell gelatin capsule or compressed into tablets. For oral therapeutic administration, the pharmaceutical composition can be incorporated with excipients and used in the form of ingestible tablets, troches, capsules, pills, wafers, and the like. Such compositions and preparations may contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Any material used in preparing any dosage unit form can be of pharmaceutically acceptable purity and substantially non-toxic in the amounts employed. In addition, the pharmaceutical composition can be incorporated into sustained-release preparations and formulations.

The pharmaceutical composition described herein may comprise one or more permeation enhancer that facilitates bioavailability of the modulator described herein. WO 2000/67798, Muranishi, 1990, Crit. Rev. Ther. Drug Carrier Systems, 7, 1, Lee et al., 1991, Crit. Rev. Ther. Drug Carrier Systems, 8, 91 are herein incorporated by reference in its entirety. In some aspects, the permeation enhancer is intestinal. In some aspects, the permeation enhancer is transdermal. In some aspects, the permeation enhancer is to facilitate crossing the brain-blood barrier. In some aspects, the permeation enhancer improves the permeability in the oral, nasal, buccal, pulmonary, vaginal, or corneal delivery model. In some aspects, the permeation enhancer is a fatty acid or a derivative thereof. In some aspects, the permeation enhancer is a surfactant or a derivative thereof. In some aspects, the permeation enhancer is a bile salt or a derivative thereof. In some aspects, the permeation enhancer is a chelating agent or a derivative thereof. In some aspects, the permeation enhancer is a non-chelating non-surfactant or a derivative thereof. In some aspects, the permeation enhancer is an ester or a derivative thereof. In some aspects, the permeation enhancer is an ether or a derivative thereof. In some specific aspects, the permeation enhancer is arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof. In one aspect, the permeation enhancer is sodium caprate (C10). In some instances, the permeation enhancer is chenodeoxycholic acid (CDCA), ursodeoxychenodeoxycholic acid (UDCA), cholic acid, dehydrocholic acid, deoxycholic acid, glucholic acid, glycholic acid, glycodeoxycholic acid, taurocholic acid taurodeoxycholic acid, sodium tauro-24,25-dihydro-fusidate or sodium glycodihydrofusidate. In some instances, the permeation enhancer is polyoxyethylene-9-lauryl ether, or polyoxyethylene-20-cetyl ether.

Further provided herein are kits comprising the modulator disclosed herein. Further provided herein are kits comprising the pharmaceutical composition disclosed herein. In some aspects, the kit comprises suitable instructions in order to perform the methods of the kit. The instructions may provide information of performing any of the methods disclosed herein, whether or not the methods may be performed using only the reagents provided in the kit.

For use in the therapeutic applications described herein, kits and articles of manufacture are also described herein. In some aspects, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic. The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The container(s) optionally have a sterile access port (for example the container is an intravenous solution bag or a vial having a stopper pierceable by a hypodermic injection needle). Such kits optionally comprise a composition with an identifying description or label or instructions relating to its use in the methods described herein.

A kit may include one or more additional containers, each with one or more of various materials (such as reagents, optionally in concentrated form, and/or devices) desirable from a commercial and user standpoint for use of the modulator described herein. Non-limiting examples of such materials include, but not limited to, buffers, diluents, filters, needles, syringes, carrier, package, container, vial and/or tube labels listing contents and/or instructions for use, and package inserts with instructions for use. In some instances, a set of instructions is included.

In some aspects, a label is on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain aspects, a pharmaceutical composition comprising the modulators provided herein and optional additional active agent is presented in a pack or dispenser device which can contain one or more unit dosage forms. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert.

Compositions containing the modulators described herein formulated in a compatible pharmaceutical carrier can also be prepared, placed in an appropriate container, and labeled for treatment of an indicated condition.

### Methods

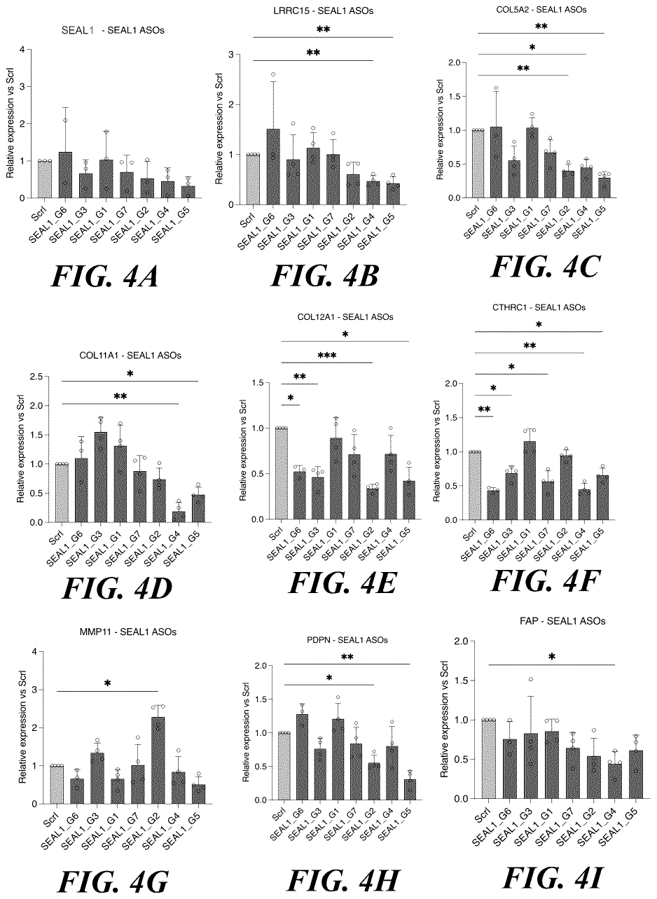

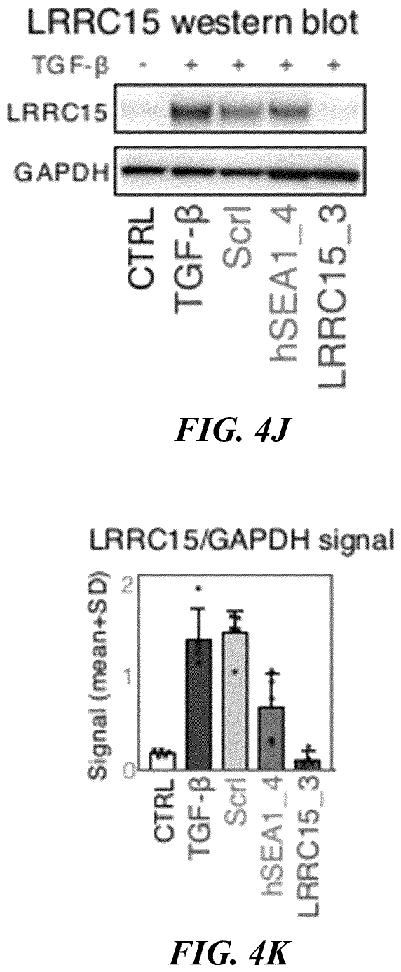

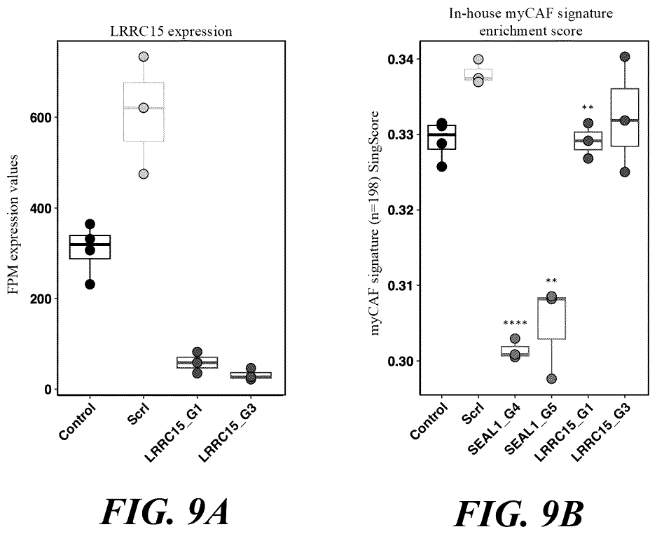

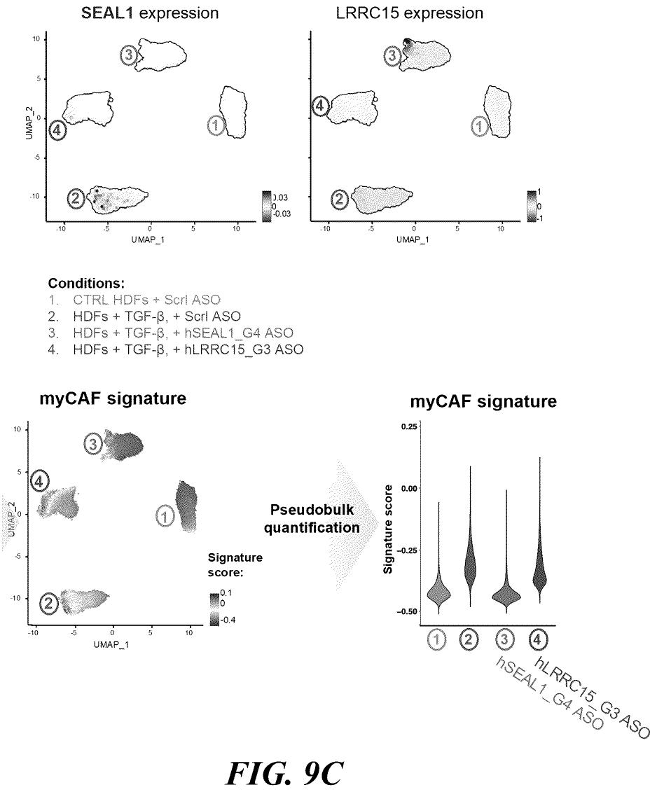

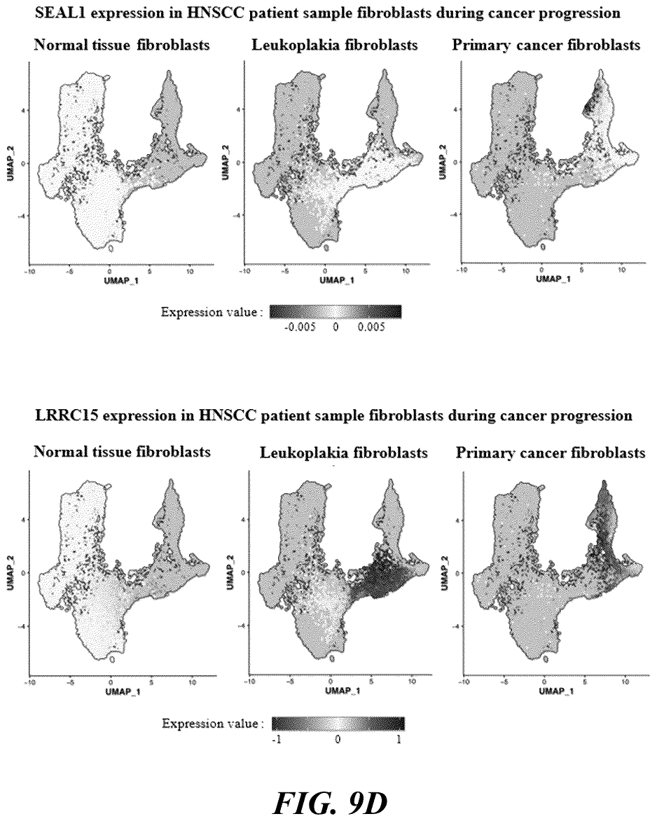

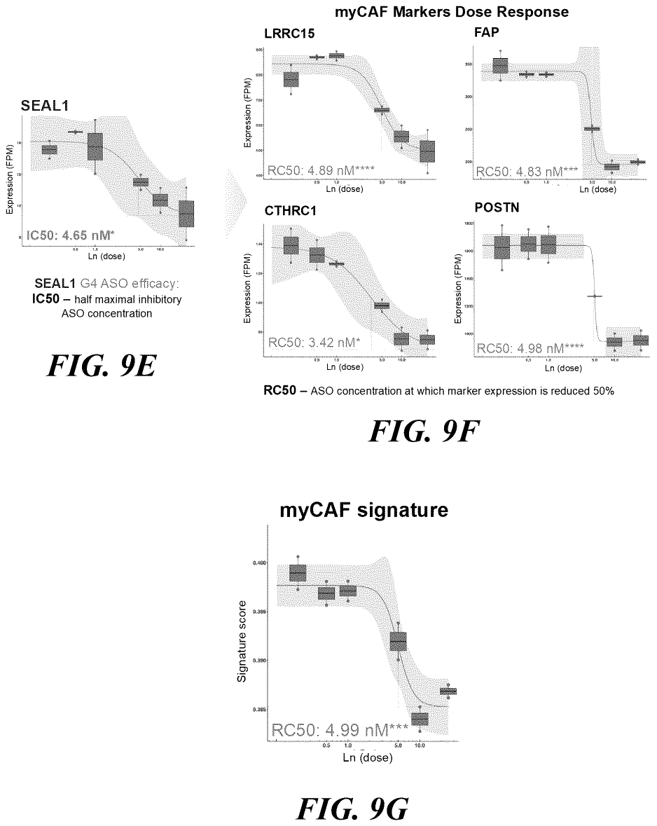

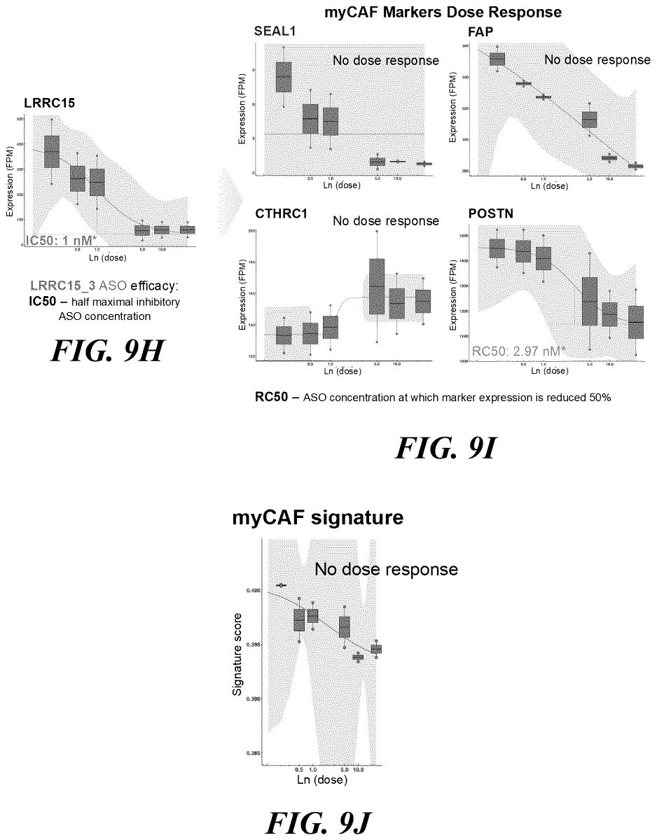

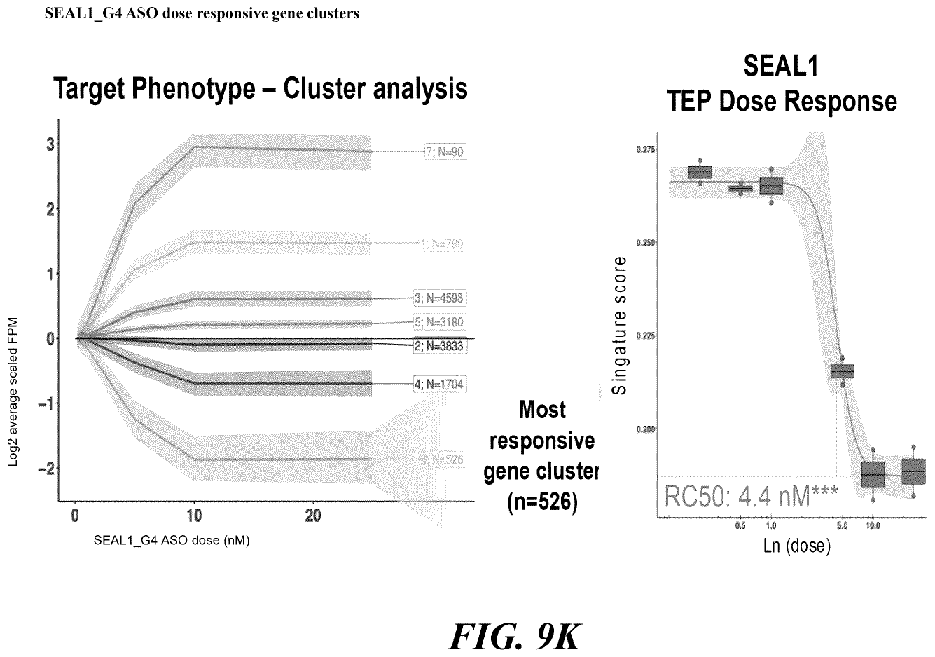

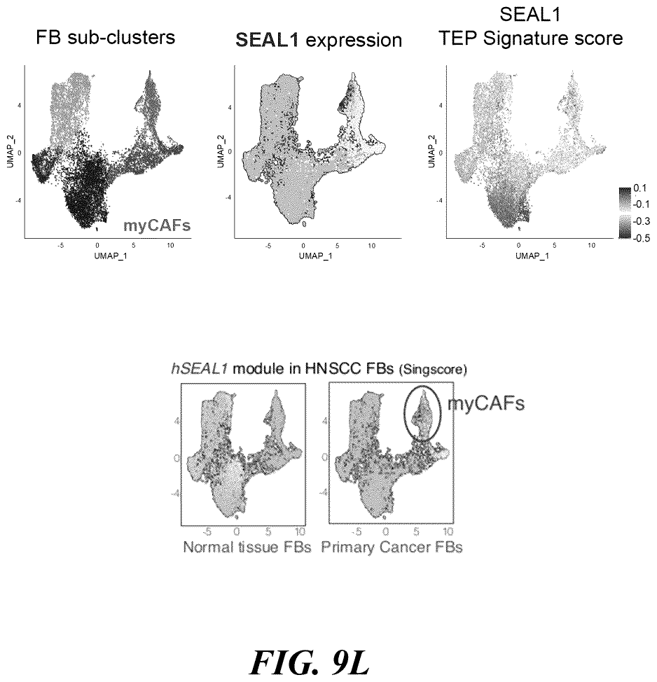

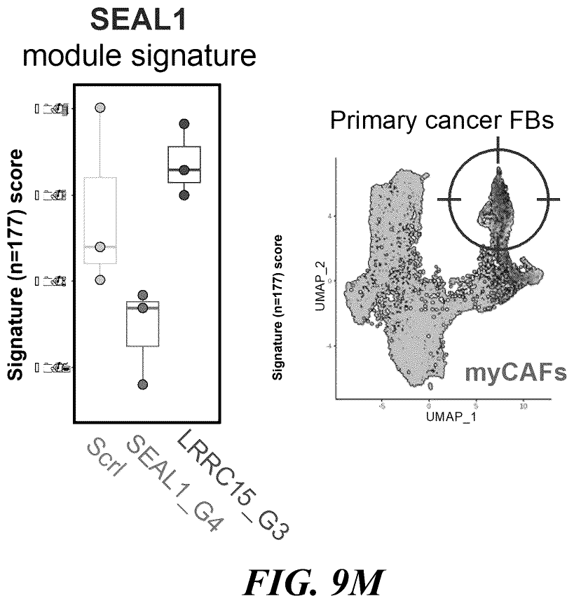

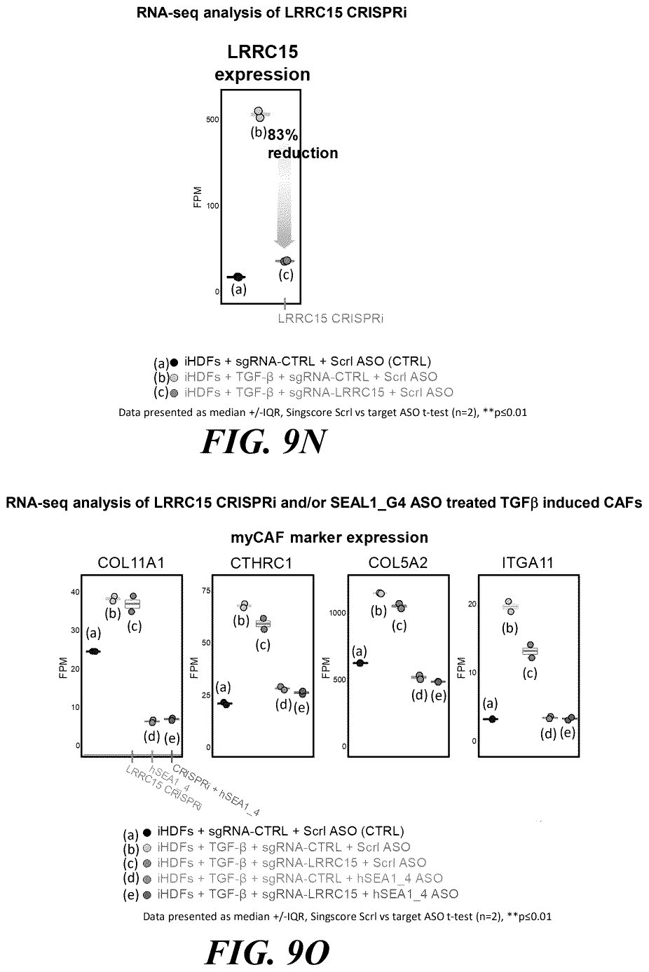

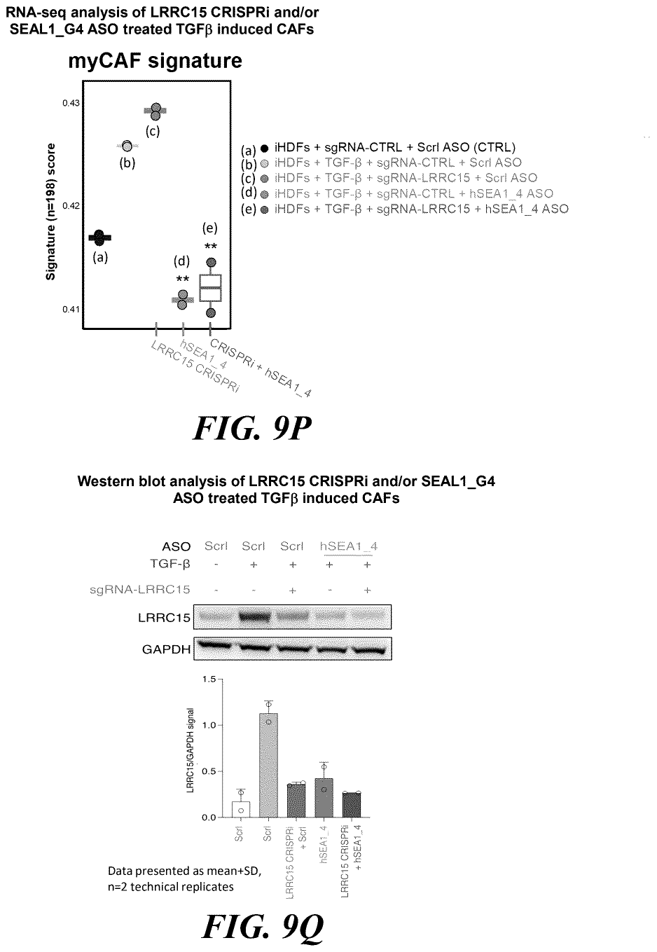

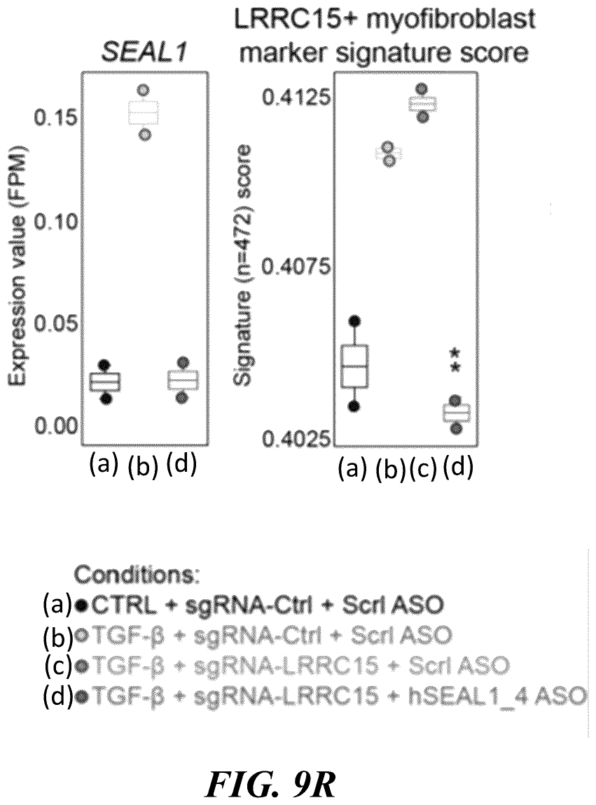

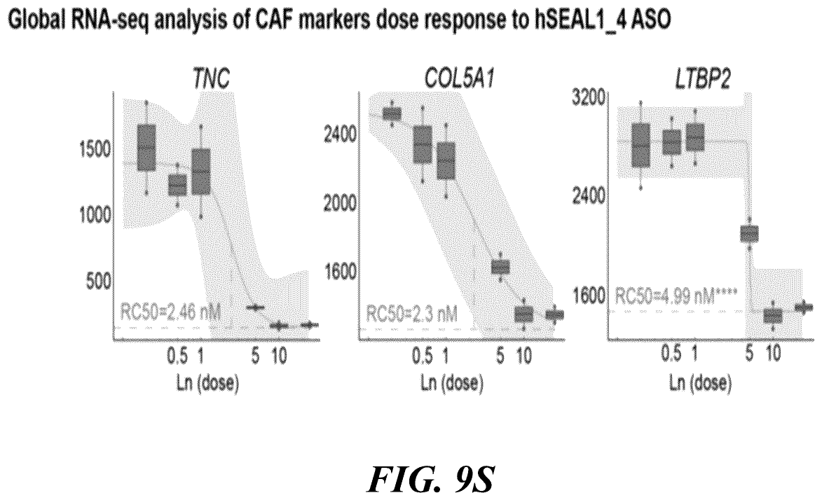

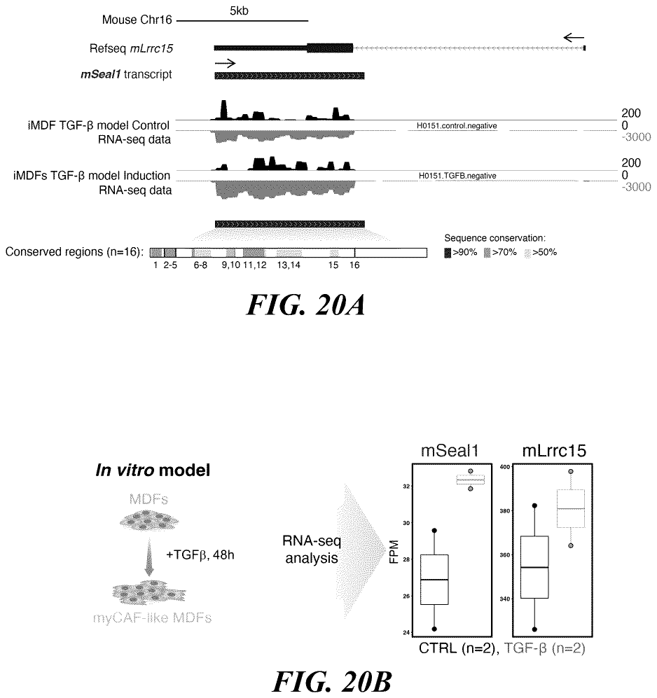

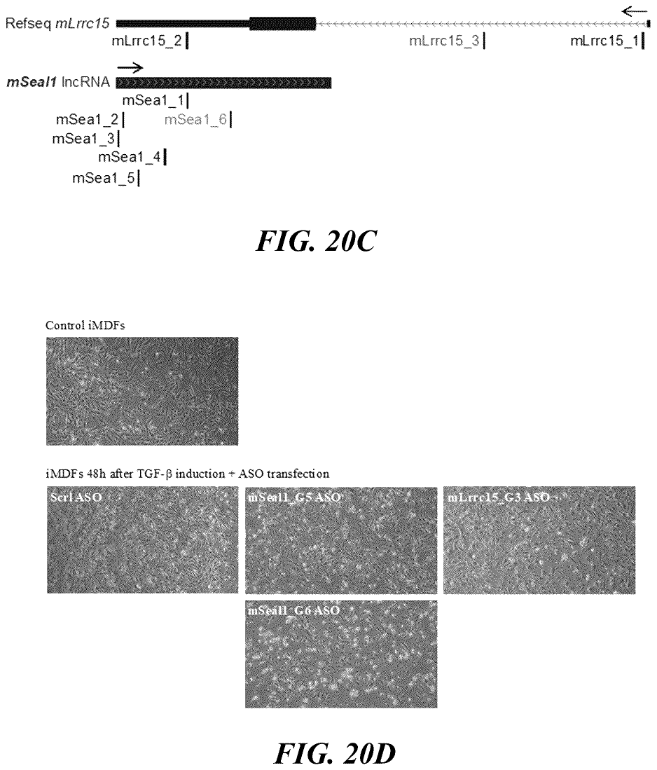

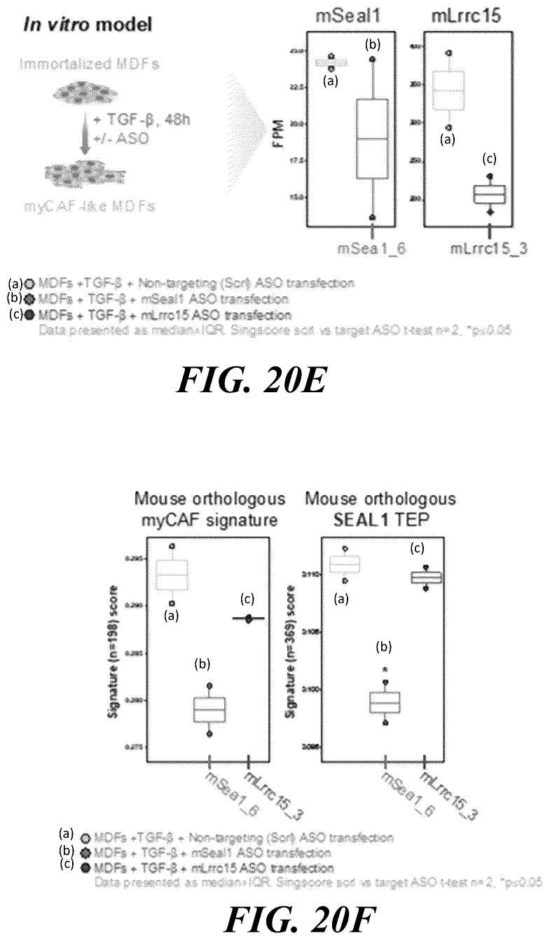

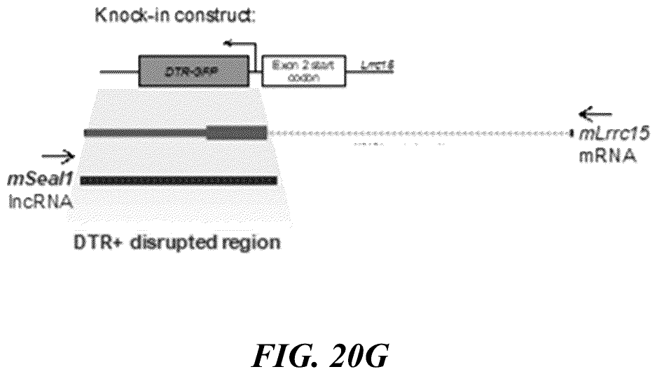

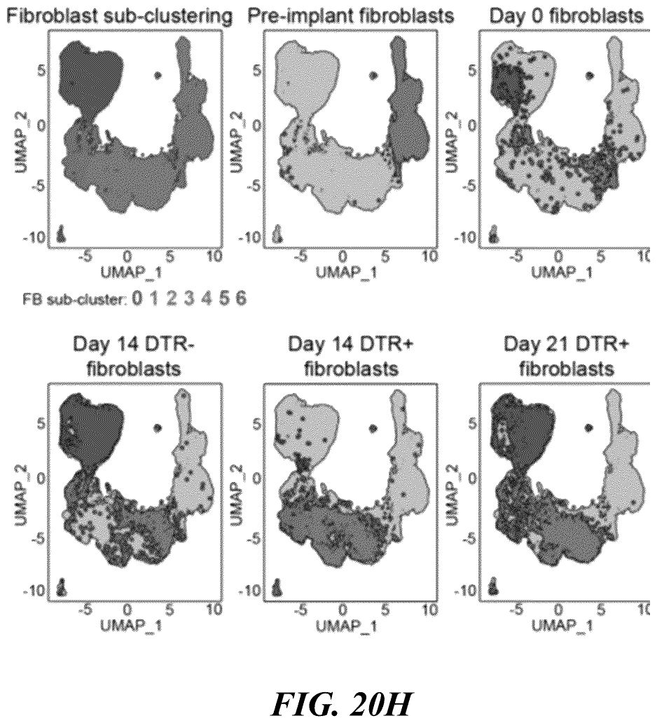

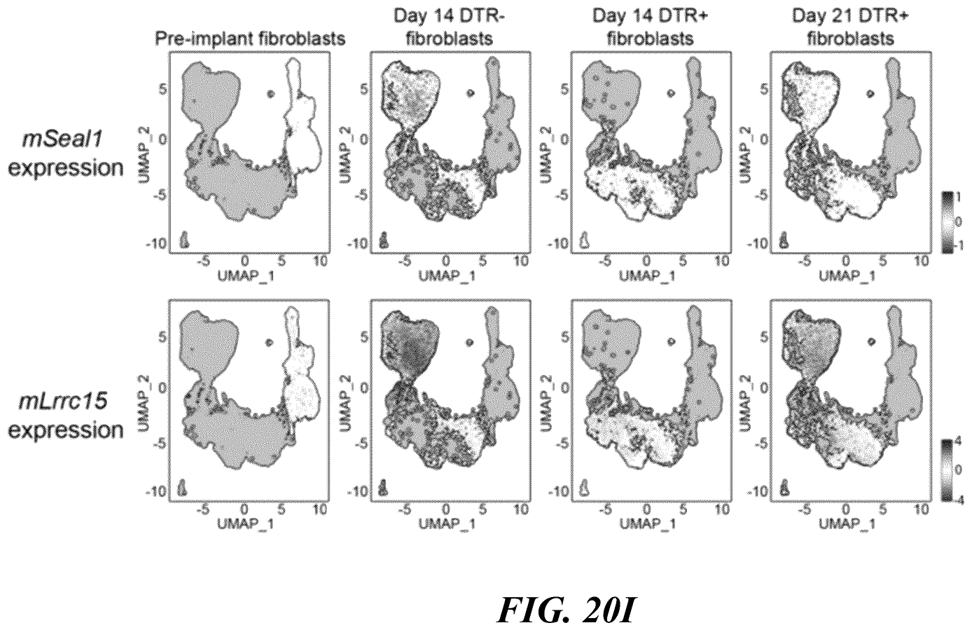

As disclosed herein, certain gene/transcription factor may be a reliable cancer-related biomarker (e.g., LRRC15, see FIG. 20I), but such gene/transcription factor may be less potent a therapeutic target to design TME/myCAF-related modulators against compared to other more promising targets (e.g., see FIG. 9H-9G, 9P). In contrast, strong evidence as disclosed herein (e.g., FIGS. 4A-4I, 6A-6I, 6K, 9E-9G, 9S, 11A-11I, 13A-13I, 13K, 14D-14J, 14L-14R, 15E-15J, 15L-15Q, and 17A-17H) support the notion that the lncRNA as disclosed herein serves as a promising therapeutic target to design TME/myCAF-related modulators against.

Accordingly, further provided herein are methods of inhibiting a growth of a solid tumor or facilitating access to a solid tumor in a subject in need thereof, the method comprising administering to the subject an effective amount of the modulator described herein or the pharmaceutical composition described herein. In some instances, the method disclosed herein directly inhibits a growth of a solid tumor. In some instances, the method disclosed herein directly facilitates access to a solid tumor. In some instances, the method disclosed herein reduces desmoplasia or fibrous tissue around tumor cells. In some instances, the method disclosed herein indirectly inhibits a growth of a solid tumor. In some instances, the method disclosed herein indirectly facilitates access to a solid tumor. In some instances, the method disclosed herein does not per se affect primary tumor growth. In some instances, the method disclosed herein inhibits the metastasis of a tumor. In some instances, the method disclosed herein improves the therapeutic outcome of an anti-tumor therapy by reducing the tumor's drug resistance. In some instances, the method disclosed herein interferes stage 0 of a tumor progression which is a pre-cancerous stage. In some instances, the method disclosed herein interferes stage I of a tumor progression when the tumor is localized to a small area and has not spread to lymph nodes or other tissues. In some instances, the method disclosed herein interferes stage II of a tumor progression when the tumor has grown without spreading. In some instances, the method disclosed herein interferes stage III of a tumor progression when the tumor has grown larger and has possibly spread to lymph nodes or other tissues. In some instances, the method disclosed herein interferes stage IV of a tumor progression when the tumor has spread to lymph nodes or other tissues. In some instances, solid tumor in subject is from a subject with cancer. Non-limiting examples of cancers are breast cancer, colorectum cancer, lung cancer, prostate cancer, bladder cancer, head and neck cancer, skin cancer (e.g., skin melanoma), pancreatic cancer, liver cancer, esophageal cancer, brain cancer, stomach cancer, gallbladder cancer, or ovarian cancer. In some aspects, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma.

Provided herein are methods of reducing expression or activity of the lncRNA disclosed herein in a subject in need thereof, the method comprising administering to the subject the modulator disclosed herein, or a pharmaceutical composition disclosed herein. Further provided herein are methods of preventing, alleviating, or treating cancer (e.g., head and neck squamous carcinoma, pancreatic cancer) or a symptom associated with cancer in a subject in need thereof, the method comprising administering to the subject an effective amount of the modulator disclosed herein or the pharmaceutical composition provided herein.

In some instances, the modulator disclosed herein or the pharmaceutical composition described herein reduces expression or activity of genes involved in ECM structure organization and/or regulation of GTPase activity in a subject. In some instances, the modulator disclosed herein or the pharmaceutical composition described herein reduces expression or activity of one or more myCAF markers in cell of a subject. In some instances, the modulator disclosed herein or the pharmaceutical composition described herein reduces expression or activity of genes that differentially express in myCAF rather than in a fibroblast that is not associated with cancer. In some instances, the modulator disclosed herein or the pharmaceutical composition described herein reduces expression or activity of genes with higher expression in myCAF than in fibroblasts that is not associated with cancer. In some instances, the modulator disclosed herein or the pharmaceutical composition described herein increases expression or activity of genes with lower expression in myCAF than in fibroblasts that is not associated with cancer. In some instances, the modulator disclosed herein or the pharmaceutical composition described herein reduces expression or activity of genes with higher expression in myCAF than in other sub-population of CAFs. In some instances, the modulator disclosed herein or the pharmaceutical composition described herein increases expression or activity of genes with lower expression in myCAF than in other sub-population of CAFs. In some instances, the modulator disclosed herein or the pharmaceutical composition described herein reduces expression or activity of genes listed in Table 1 or Table 13. In some instances, the modulator disclosed herein or the pharmaceutical composition described herein reduces expression or activity of an ECM-modulatory gene. In some instances, the modulator described herein or the pharmaceutical composition disclosed herein reduces expression or activity of one or more ECM-modulatory genes in cell or tissue of a subject. Non-limiting examples of ECM-modulatory genes are LRRC15 (NM_001135057), MMP11 (NM_005940), COL11A1 (NM_080629), C1QTNF3 (NM_030945), CTHRC1 (NM_138455), COL12A1 (NM 004370), COL10A1 (NM 00493), COL5A2 (NM_000393), THBS2 (NM_003247), AEBP1 (NM_001129), ITGA11 (NM 001004439), PDPN (NM 006474), FAP (NM_004460), COL8A1 (NM_001850), COL1A1 (NM_000088), COL1A2 (NM 000089), FN1 (NM_212482), POSTN (NM_006475), or variations thereof.

In some instances, the modulator described herein or the pharmaceutical composition described herein restores an expression or activity of at least one or more genes (e.g., myCAF markers, ECM-modulatory genes) to a level comparable to a healthy cell. In some instances, the modulator described herein or the pharmaceutical composition described herein restores an expression or activity of at least one or more genes (e.g., myCAF markers, ECM-modulatory genes) by at least about 20%, at least about 30%, at least about 40%, at least about 50%, at least about 60%, at least about 70%, at least about 80%, or at least about 90% comparable to a healthy cell.

In some instances, the modulator described herein or the pharmaceutical composition described herein is encapsulated in a liposome or coupled with a nanoparticle. In some instances, the modulator described herein or the pharmaceutical composition described herein is encoded by a transgene in an expression vector. In some instances, the modulator or the synthetic polynucleic acid described herein is encapsulated in an extracellular vesicle.

In some instances, the modulator or the pharmaceutical composition comprising the modulator is administered systemically. In some instances, the modulator or the pharmaceutical composition comprising the modulator is administered locally. In some instances, the modulator or the pharmaceutical composition comprising the modulator is administered intratracheally, orally, nasally, intravenously, intraperitoneally, or intramuscularly. In some instances, the modulator or the pharmaceutical composition comprising the modulator is administered via a targeted delivery to a tumor tissue (e.g., head and neck squamous carcinoma) of the subject.

For delivery to the target cell or tissue (e.g., head and neck squamous carcinoma), the modulator described herein can non-covalently bind an excipient to form a complex. The excipient can be used to alter biodistribution after delivery, to enhance uptake, to increase half-life or stability of the strands in the modulator described herein (e.g., improve nuclease resistance), and/or to increase targeting to a particular cell or tissue type. Exemplary excipients include but are not limited to a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include but are not limited to polyamines (e.g., polylysine, polyarginine, polyamidoamine, and polyethylene imine); cholesterol; a dendrimer (e.g., a polyamidoamine (PAMAM) dendrimer); a serum protein (e.g., human serum albumin (HSA) or low-density lipoprotein (LDL)); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly(L-lactide-co-glycolic) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; or a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

In some aspects, administration of the modulator or the pharmaceutical composition comprising the modulator is a targeted delivery to a tumor of the subject. In some instances, the targeted delivery is via a local application. In some instances, the targeted delivery is via conjugation or coupling of the modulator to one or more specific targeting or binding moieties that target the tumor (e.g., tumor cell, a cell in the tumor microenvironment, specific extracellular matrix in the tumor microenvironment, etc.).

Further provided herein are methods of diagnosing or monitoring a cancer or a cancer development in a subject, the method comprising: (a) obtaining a sample from the subject; (b) detecting an expression and/or an activity of a biomarker from the sample, wherein the biomarker comprises an lncRNA described herein (e.g., an lncRNA transcribed from a genomic region or a subset thereof listed in Table 10 or Table 12, an lncRNA comprising a sequence or a fragment thereof listed in Table 11, an lncRNA sharing at least 80%, 85%, 90% or 95% sequence homology with a full-length sequence listed in Table 11, or an lncRNA sharing at least 80%, 85%, 90% or 95% sequence homology with a fragment of a sequence listed in Table 11); and (c) diagnosing the subject with cancer or to have a high/higher chance to contract cancer if the expression and/or the activity of the biomarker is at least 50%, at least 60%, at least 70%, at least 80%, or at least 90% higher when compared to a control. In some aspects, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry. In some aspects, the detecting comprises any sequencing-based methods that include but are not limited to bulk RNA sequencing, single cell or single nucleus RNA sequencing, DNA sequencing, DNA methylation profiling (e.g., for example, average % methylated CpG along the lncRNA locus described herein or the methylation status of specific CpGs within the lncRNA locus described herein), sc- or snATAC-seq or bulk ATAC-seq, DNase I assay for accessibility, CUT&RUN or ChIP-seq for specific histone marks (e.g., H3K27ac, H4K16ac, H3K9me3, H3K27me3, H3K4me1, H3K4me3, etc.). In some aspects, the biomarker further comprise INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1, or a gene from Table 1, Table 13, or Table 14. In some aspects, the detecting comprises detecting the activity and/or expression of transcription factors TEAD2, RUNX1, RUNX2, NFATC4. In some aspects, the method further comprises (d) administering the subject the modulator described herein, or the pharmaceutical composition described herein. In some aspects, the cancer is a solid tumor. In some aspects, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma. In some instances, the sample is a tumor fibroblast sample.

Further provided herein are methods of diagnosing or monitoring a cancer or a cancer development in a subject, the method comprising: (a) obtaining a sample from the subject; (b) detecting an expression and/or an activity of a biomarker from the sample, wherein the biomarker comprises INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGAI1, PDPN, FAP, COL8A1, TEAD2, RUNX1, RUNX2, NFATC4, or a gene from Table 1, Table 13, or Table 14, or a combination thereof; and (c) diagnosing the subject with cancer or to have a high/higher chance to contract cancer if the expression and/or the activity of the biomarker is at least 50%, at least 60%, at least 70%, at least 80%, or at least 90% higher when compared to a control. In some aspects, the detecting comprises using SI nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry. In some aspects, the detecting comprises any sequencing-based methods that include but are not limited to bulk RNA sequencing, single cell or single nucleus RNA sequencing, DNA sequencing, DNA methylation profiling (e.g., for example, average % methylated CpG along the lncRNA locus described herein or the methylation status of specific CpGs within the InRNA locus described herein), sc- or snATAC-seq or bulk ATAC-seq, DNase I assay for accessibility, CUT&RUN or ChIP-seq for specific histone marks (e.g., H3K27ac, H4K16ac, H3K9me3, H3K27me3, H3K4me1, H3K4me3, etc.). In some aspects, the method further comprises (d) administering the subject the modulator described herein, or the pharmaceutical composition described herein. In some aspects, the cancer is a solid tumor. In some aspects, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma. In some instances, the sample is a tumor fibroblast sample.

Further provided herein are methods of diagnosing or monitoring a cancer or a cancer development in a subject, the method comprising: (a) obtaining a tumor fibroblast sample from the subject; (b) counting a number of fibroblasts that exhibit a higher expression of a biomarker, wherein the biomarker comprises a biomarker from the sample, wherein the biomarker comprises an lncRNA described herein (e.g., (e.g., an lncRNA transcribed from a genomic region or a subset thereof listed in Table 10 or Table 12, an lncRNA comprising a sequence or a fragment thereof listed in Table 11, an lncRNA sharing at least 80%, 85%, 90% or 95% sequence homology with a full-length sequence listed in Table 11, or an lncRNA sharing at least 80%, 85%, 90% or 95% sequence homology with a fragment of a sequence listed in Table 11); and (c) diagnosing the subject with cancer or to have a high/higher chance to contract cancer if the number of fibroblasts that exhibit a higher expression of the biomarker is higher than the number in a control. In some aspects, the counting in (b) is performed by fluorescence-activated cell sorting (FACS), single cell or single nucleus RNA sequencing combined with cell sorting. In some aspects, the biomarker further comprise INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1, or a gene from Table 1, Table 13, or Table 14. In some aspects, the detecting comprises detecting the activity and/or expression of transcription factors TEAD2, RUNX1, RUNX2, NFATC4. In some aspects, the method further comprises (d) administering the subject the modulator described herein, or the pharmaceutical composition described herein. In some aspects, the cancer is a solid tumor. In some aspects, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma.

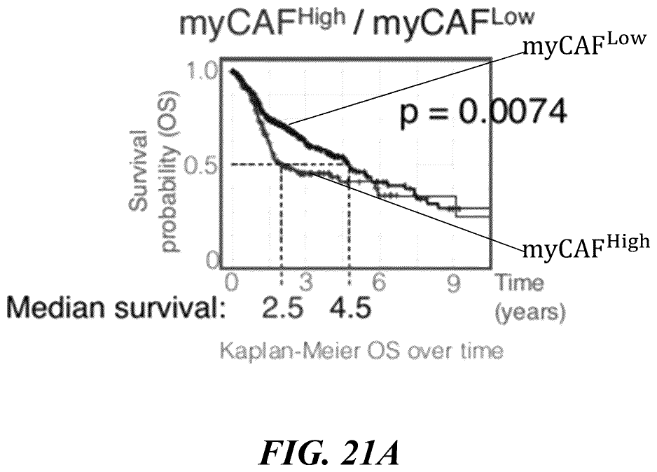

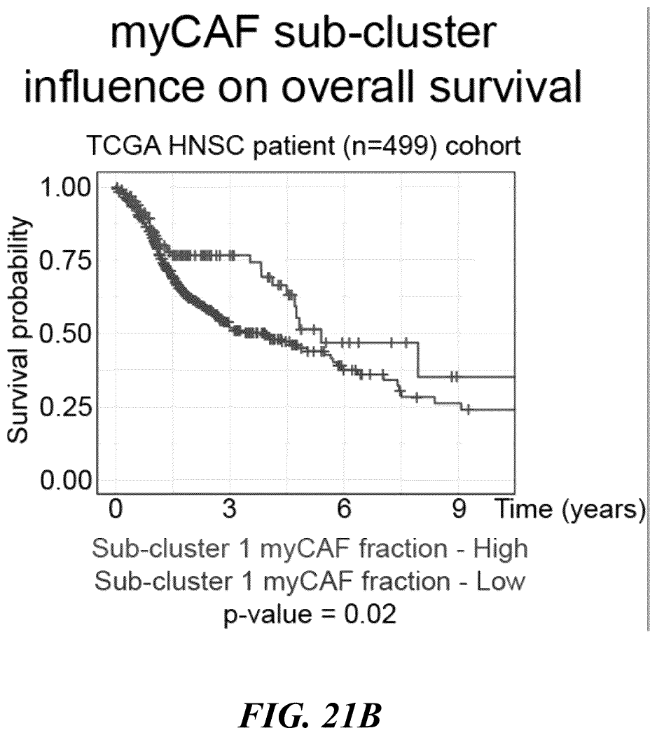

As disclosed herein, the lncRNA disclosed herein is associated with poor overall patient survival by promoting tumor progression, cancer cell migration and metastasis, resistance to standard-of-care treatments, immune suppression as well as immunotherapy (PD-L1/PD-1 pathway blockade) failure. Accordingly, further provided herein are methods of predicting severity and progression/metastasis of a cancer in a subject, the method comprising: (a) obtaining a sample from the subject; (b) detecting an expression and/or an activity of a biomarker from the sample, wherein the biomarker comprises a biomarker from the sample, wherein the biomarker comprises an lncRNA described herein (e.g., (e.g., an lncRNA transcribed from a genomic region or a subset thereof listed in Table 10 or Table 12, an lncRNA comprising a sequence or a fragment thereof listed in Table 11, an lncRNA sharing at least 80%, 85%, 90% or 95% sequence homology with a full-length sequence listed in Table 11, or an lncRNA sharing at least 80%, 85%, 90% or 95% sequence homology with a fragment of a sequence listed in Table 11); and (c) diagnosing the subject to have a more severe or a progression of the cancer if the expression and/or the activity of the biomarker is at least 50%, at least 60%, at least 70%, at least 80%, or at least 90% higher when compared to a control. In some aspects, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry. In some aspects, the detecting comprises any sequencing-based methods that include but are not limited to bulk RNA sequencing, single cell or single nucleus RNA sequencing, DNA sequencing, DNA methylation profiling (e.g., for example, average % methylated CpG along the lncRNA locus described herein or the methylation status of specific CpGs within the InRNA locus described herein), sc- or snATAC-seq or bulk ATAC-seq, DNase I assay for accessibility, CUT&RUN or ChIP-seq for specific histone marks (e.g., H3K27ac, H4K16ac, H3K9me3, H3K27me3, H3K4me1, H3K4me3, etc.). In some aspects, the biomarker further comprise INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1, or a gene from Table 1, Table 13, or Table 14, as supported by e.g., FIG. 21A. In some aspects, the detecting comprises detecting the activity and/or expression of transcription factors TEAD2, RUNX1, RUNX2, NFATC4. In some aspects, the method further comprises (d) administering the subject the modulator described herein, or the pharmaceutical composition described herein. In some aspects, the cancer is a solid tumor. In some aspects, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma. In some instances, the sample is a tumor fibroblast sample.

Further provided herein are methods of predicting severity and progression/metastasis of a cancer in a subject, the method comprising: (a) obtaining a sample from the subject; (b) detecting an expression and/or an activity of a biomarker from the sample, wherein the biomarker comprises a biomarker from the sample, wherein the biomarker comprises INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1, TEAD2, RUNX1, RUNX2, NFATC4, or a gene from Table 1, Table 13, or Table 14, or a combination thereof, and (c) diagnosing the subject to have a more severe or a progression of the cancer if the expression and/or the activity of the biomarker is at least 50%, at least 60%, at least 70%, at least 80%, or at least 90% higher when compared to a control. In some aspects, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry. In some aspects, the detecting comprises any sequencing-based methods that include but are not limited to bulk RNA sequencing, single cell or single nucleus RNA sequencing, DNA sequencing, DNA methylation profiling (e.g., for example, average % methylated CpG along the lncRNA locus described herein or the methylation status of specific CpGs within the InRNA locus described herein), sc- or snATAC-seq or bulk ATAC-seq, DNase I assay for accessibility, CUT&RUN or ChIP-seq for specific histone marks (e.g., H3K27ac, H4K16ac, H3K9me3, H3K27me3, H3K4me1, H3K4me3, etc.). In some aspects, the method further comprises (d) administering the subject the modulator described herein, or the pharmaceutical composition described herein. In some aspects, the cancer is a solid tumor. In some aspects, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma. In some instances, the sample is a tumor fibroblast sample.

As disclosed herein, the number of a subset of fibroblasts enriched with the lncRNA disclosed herein is a reliable prognosis readout (see, e.g., FIG. 21B). Accordingly, further provided herein are methods of predicting severity and progression/metastasis of a cancer in a subject, the method comprising: (a) obtaining a tumor fibroblast sample from the subject; (b) counting a number of fibroblasts that exhibit a higher expression of a biomarker, wherein the biomarker comprises a biomarker from the sample, wherein the biomarker comprises an lncRNA described herein (e.g., (e.g., an lncRNA transcribed from a genomic region or a subset thereof listed in Table 10 or Table 12, an lncRNA comprising a sequence or a fragment thereof listed in Table 11, an lncRNA sharing at least 80%, 85%, 90% or 95% sequence homology with a full-length sequence listed in Table 11, or an lncRNA sharing at least 80%, 85%, 90% or 95% sequence homology with a fragment of a sequence listed in Table 11); and (c) diagnosing the subject to have a more severe or a progression of the cancer if the number of fibroblasts that exhibit a higher expression of the biomarker is higher than the number in a control. In some aspects, the counting in (b) is performed by fluorescence-activated cell sorting (FACS), single cell or single nucleus RNA sequencing combined with cell sorting. In some aspects, the biomarker further comprise INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1, or a gene from Table 1, Table 13, or Table 14. In some aspects, the detecting comprises detecting the activity and/or expression of transcription factors TEAD2, RUNX1, RUNX2, NFATC4. In some aspects, the method further comprises (d) administering the subject the modulator described herein, or the pharmaceutical composition described herein. In some aspects, the cancer is a solid tumor. In some aspects, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma.

Further provided herein are methods of monitoring an efficacy or therapeutic resistance of a therapy treating a cancer in a subject, the method comprising: (a) obtaining a sample from the subject; (b) detecting an expression and/or an activity of a biomarker from the sample, wherein the biomarker comprises an lncRNA described herein (e.g., an lncRNA transcribed from a genomic region or a subset thereof listed in Table 10 or Table 12, an lncRNA comprising a sequence or a fragment thereof listed in Table 11, an lncRNA sharing at least 80%, 85%, 90% or 95% sequence homology with a full-length sequence listed in Table 11, or an lncRNA sharing at least 80%, 85%, 90% or 95% sequence homology with a fragment of a sequence listed in Table 11); and (c) concluding the therapy treating the cancer is effective or is less likely to develop therapeutic resistance if the expression and/or the activity of the biomarker is at least 10%, at least 20%, at least 30%, at least 40%, at least 50%, at least 60%, at least 70%, at least 80%, or at least 90% reduced by the therapy. In some aspects, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry. In some aspects, the detecting comprises any sequencing-based methods that include but are not limited to bulk RNA sequencing, single cell or single nucleus RNA sequencing, DNA sequencing, DNA methylation profiling (e.g., for example, average % methylated CpG along the lncRNA locus described herein or the methylation status of specific CpGs within the InRNA locus described herein), sc- or snATAC-seq or bulk ATAC-seq, DNase I assay for accessibility, CUT&RUN or ChIP-seq for specific histone marks (e.g., H3K27ac, H4K16ac, H3K9me3, H3K27me3, H3K4me1, H3K4me3, etc.). In some aspects, the biomarker further comprise INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1,or a gene from Table 1, Table 13, or Table 14. In some aspects, the detecting comprises detecting the activity and/or expression of transcription factors TEAD2, RUNX1, RUNX2, NFATC4. In some aspects, the method further comprises (d) administering the subject the modulator described herein, or the pharmaceutical composition described herein. In some aspects, the cancer is a solid tumor. In some aspects, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma. In some instances, the sample is a tumor fibroblast sample.

Further provided herein are methods of monitoring an efficacy or therapeutic resistance of a therapy treating a cancer in a subject, the method comprising: (a) obtaining a sample from the subject; (b) detecting an expression and/or an activity of a biomarker from the sample, wherein the biomarker comprises INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1, TEAD2, RUNX1, RUNX2, NFATC4, or a gene from Table 1, Table 13, or Table 14, or a combination thereof; and (c) concluding the therapy treating the cancer is effective or is less likely to develop therapeutic resistance if the expression and/or the activity of the biomarker is at least 10%, at least 20%, at least 30%, at least 40%, at least 50%, at least 60%, at least 70%, at least 80%, or at least 90% reduced by the therapy. In some aspects, the detecting comprises using S1 nuclease protection assay, microarray analysis, polymerase chain reaction (PCR), hybridization technologies, reverse transcriptase polymerase chain reaction (RT-PCR), Northern blot, serial analysis of gene expression (SAGE), immunoassay, and/or mass spectrometry. In some aspects, the detecting comprises any sequencing-based methods that include but are not limited to bulk RNA sequencing, single cell or single nucleus RNA sequencing, DNA sequencing, DNA methylation profiling (e.g., for example, average % methylated CpG along the lncRNA locus described herein or the methylation status of specific CpGs within the InRNA locus described herein), sc- or snATAC-seq or bulk ATAC-seq, DNase I assay for accessibility, CUT&RUN or ChIP-seq for specific histone marks (e.g., H3K27ac, H4K16ac, H3K9me3, H3K27me3, H3K4me1, H3K4me3, etc.). In some aspects, the method further comprises (d) administering the subject the modulator described herein, or the pharmaceutical composition described herein. In some aspects, the cancer is a solid tumor. In some aspects, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma. In some instances, the sample is a tumor fibroblast sample.

Further provided herein are methods of monitoring an efficacy or therapeutic resistance of a therapy treating a cancer in a subject, the method comprising: (a) obtaining a tumor fibroblast sample from the subject; (b) counting a number of fibroblasts that exhibit a higher expression of a biomarker, wherein the biomarker comprises a biomarker from the sample, wherein the biomarker comprises an lncRNA described herein (e.g., (e.g., an lncRNA transcribed from a genomic region or a subset thereof listed in Table 10 or Table 12, an lncRNA comprising a sequence or a fragment thereof listed in Table 11, an lncRNA sharing at least 80%, 85%, 90% or 95% sequence homology with a full-length sequence listed in Table 11, or an lncRNA sharing at least 80%, 85%, 90% or 95% sequence homology with a fragment of a sequence listed in Table 11); and (c) concluding the therapy treating the cancer is effective or is less likely to develop therapeutic resistance if the number of fibroblasts that exhibit a higher expression of the biomarker is reduced by the therapy. In some aspects, the counting in (b) is performed by fluorescence-activated cell sorting (FACS), single cell or single nucleus RNA sequencing combined with cell sorting. In some aspects, the biomarker further comprise INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1, or a gene from Table 1, Table 13, or Table 14. In some aspects, the detecting comprises detecting the activity and/or expression of transcription factors TEAD2, RUNX1, RUNX2, NFATC4. In some aspects, the method further comprises (d) administering the subject the modulator described herein, or the pharmaceutical composition described herein. In some aspects, the cancer is a solid tumor. In some aspects, the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma.

### Certain Terminology

The term “noncoding RNA” as used herein, can refer to RNA species that are not translated into protein. The term “long noncoding RNA” or “lncRNA” as used herein, refers to a noncoding RNA that is at least 100, 200, 300, 400, 500, 600, 700, 800, 900, or 1000 nucleotides long. In some instances, “long noncoding RNA” or “lncRNA” comprises at least 1, 2, 3, 4, 5, 6, 7, 8, 9, or 10 exons.

The term “myCAF” or “myofibroblast CAF” or “myofibroblast cancer-associated fibroblasts” as used herein, can refer to cancer-associated fibroblasts (CAFs) that is associated with collagen-fibril organization, extracellular matrix (ECM) organization, tumor innervation and/or tissue development in tumor microenvironment. In some instanced “myCAF” or “myofibroblast CAF” or “myofibroblast cancer-associated fibroblasts” exhibits a higher expression of an ECM-modulatory gene when compared to a fibroblast that is not associated with cancer.

The term “healthy cell” as used herein refers to in healthy cells of a healthy individual. In some instances, it refers to a cell of the same subject but before contracting any disorders, such as cancer.

The term “normal tissues” as used herein refers to in healthy tissues of a healthy individual. In some instances, it refers to a tissue of the same subject but before contracting any disorders, such as cancer.

The term “nucleic acid editing or modifying moiety,” as used herein, can refer to a moiety that edits or cleaves the target nucleic acid. It can also refer to a moiety that suppresses the transcription of the target nucleic acid.

The term “nucleic acid analogue,” as used herein, can refer to compounds which are analogous (structurally similar) to naturally occurring nucleic acid (see, e.g., Freier & Altmann; Nucl. Acid. Res., 1997, 25, 4429-4443 and Uhlmann; Curr. Opinion in Drug Development, 2000, 3(2), 293-213), and examples of suitable nucleic acid analogues are provided by WO2007031091, which are hereby incorporated by reference.

The term “gapmer” is a chimeric nucleic acid that contains a central sequence of phosphorothioate DNA nucleotides (“DNA gap”) flanked by sequences of modified RNA residues at either end to protect the DNA gap from nuclease degradation, whereas the central DNA gap region allows RNase-H-mediated cleavage of the target RNA. Gapmer has an internal region having a plurality of nucleosides which is capable of recruiting RNase H activity, such as RNaseH, which region is positioned between external wings at either end, having one or more nucleosides, wherein the nucleosides comprising the internal region are chemically distinct from the nucleoside or nucleosides comprising the external wings.

A “locked nucleic acid” or “LNA” is often referred to as inaccessible RNA, and is a modified RNA nucleobase. The ribose moiety of an LNA nucleobase is modified with an extra bridge connecting the 2′ oxygen and 4′ carbon. An LNA oligonucleotide offers substantially increased affinity for its complementary strand, compared to traditional DNA or RNA oligonucleotides.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers to the primary structure of the molecule. Thus, the term includes triple-, double- and single-stranded DNA, as well as triple-, double- and single-stranded RNA. In some aspects, it also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Vials, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, RNA, double- and single-stranded DNA, as well as double- and single-stranded RNA, microRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2′-aminoadenosine, 2′-thiothymidine, inosine, pyrrolo-pyrimidine, 3′-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, and 2′-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30: 1911-1918.

The terms “siRNA” and “short interfering RNA” are interchangeable and refer to single-stranded or double-stranded RNA molecules that are capable of inducing RNA interference. In some aspects, siRNA molecules have a duplex region that is between 18 and 30 nucleotides in length.

The terms “microRNA,” “miRNA,” and MiR” are interchangeable and refer to endogenous or artificial non-coding RNAs that are capable of regulating gene expression. It is believed that miRNAs function via RNA interference.

The terms “piRNA” and “Piwi-interacting RNA” are interchangeable and refer to a class of small RNAs involved in gene silencing. In some instances, piRNA molecules are between 26 and 31 nucleotides in length.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against one or more lncRNAs of the disclosure.

The term “complementary” and “complementarity” are interchangeable and refer to the ability of polynucleotides to form nucleotides with one another. In some instances, nucleotides are formed by hydrogen bonds between nucleotide units in antiparallel polynucleotide strands or regions. Complementary polynucleotide strands or regions can base pair in the Watson-Crick manner (e.g., A to T, A to U, C to G). 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

“Administering”, as it applies in the present disclosure, refers to contact of an effective amount of a modulator of one or more lncRNAs of the disclosure, to the subject. Administering a nucleic acid, such as a microRNA, siRNA, piRNA, snRNA, or antisense nucleic acid, to a cell comprises transducing, transfecting, electroporating, translocating, fusing, phagocytosing, shooting or ballistic methods, or any means by which a nucleic acid can be transported across a cell membrane.

“Pharmaceutically acceptable excipient or carrier” refers to an excipient that may optionally be included in the compositions of the disclosure and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

An “effective amount” of modulator of one or more lncRNAs of the disclosure (e.g., microRNA, siRNA, piRNA, snRNA, antisense nucleic acid, ribozyme, or small molecule inhibitor, CRISPRs etc.) is an amount sufficient to effect beneficial or desired results, such as an amount that inhibits the activity of a lncRNA, for example by interfering with transcription. An effective amount can be administered in one or more administrations, applications, or dosages.

By “therapeutically effective dose or amount” of a modulator of one or more lncRNAs of the disclosure is intended an amount that, when administered as described herein, brings about a positive therapeutic response. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Alternatively, homology can be determined by readily available computer programs or by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art.

As used herein, a “sample” refers to a sample of tissue or fluid isolated from a subject, including but not limited to, for example, urine, blood, plasma, serum, fecal matter, bone marrow, bile, spinal fluid, lymph fluid, samples of the skin, external secretions of the skin, respiratory, intestinal, and genitourinary tracts, tears, saliva, milk, blood cells, organs, biopsies, and also samples containing cells or tissues derived from the subject and grown in culture, and in vitro cell culture constituents, including but not limited to, conditioned media resulting from the growth of cells and tissues in culture, recombinant cells, stem cells, and cell components.

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

“Diagnosis” as used herein generally includes determination as to whether a subject is likely affected by a given disease, disorder or dysfunction of the disclosure. The skilled artisan often makes a diagnosis on the basis of one or more diagnostic indicators (e.g., a biomarker), the presence, absence, or amount of which is indicative of the presence or absence of the disease, disorder or dysfunction.

The term “comprising” (and related terms such as “comprise” or “comprises” or “having” or “including”) is not intended to exclude that in other certain instances, for example, an instance of any composition of matter, composition, method, or process, or the like, described herein, “consist of” or “consist essentially of” the described features.

The term “subject” or “patient,” as used herein, generally encompasses organisms such as mammals. Examples of mammals include, but are not limited to, any member of the mammalian class: humans, non-human primates, such as chimpanzees, and other apes and monkey species; farm animals, such as cattle, horses, sheep, goats, swine; domestic animals, such as rabbits, dogs, and cats; laboratory animals, including rodents, such as rats, mice and guinea pigs, and the like.

In one aspect, the mammal is a human.

The term “treatment” or “treating,” as used herein, are used interchangeably. These terms generally refer to an approach for obtaining beneficial or desired results including but not limited to therapeutic benefit and/or a prophylactic benefit. In some instances, the term refers to eradication of the underlying disorder being treated. In other instances, the term refers to the eradication of one or more of the physiological symptoms associated with the underlying disorder such that an improvement is observed in the patient, notwithstanding that the patient is still afflicted with the underlying disorder.

The term “alleviating” or “alleviate,” as used herein, refers to amelioration, improving, or stalling the further progression of the underlying disorder being treated. In other instances, the term refers to the amelioration, improving, or stalling the further progression of one or more of the physiological symptoms associated with the underlying disorder such that an improvement is observed in the patient, notwithstanding that the patient is still afflicted with the underlying disorder.

The term “preventing” or “prevent,” as used herein, refers to the situation where the compositions disclosed herein are administered to a patient at risk of developing a particular disease, or to a patient reporting one or more of the physiological symptoms of a disease, even though a diagnosis of this disease has not been made.

Whenever the term “at least,” “more than,” or “less than” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

The term “a,” “an” and “the” include plural referents unless the content clearly dictates otherwise. Thus, for example, reference to “a lncRNA” includes a mixture of two or more lncRNAs, and the like.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The term “about,” particularly in reference to a given quantity, is meant to encompass deviations of plus or minus ten percent.

### EXAMPLES

The following is a description of various non-limiting examples of methods and materials used in the studies, and are put forth so as to provide those of ordinary skill in the art with a complete disclosure and description of how to make and use the present disclosure, and are not intended to limit the scope of the disclosure nor are they intended to represent that the experiments below were performed and are all of the experiments that may be performed.

Efforts have been made to ensure accuracy with respect to numbers used (e.g., amounts, percentages, etc.), but some experimental errors and deviations should be accounted for.

### Example 1: Discovery of Target Long Noncoding RNA (OncRNA) Candidates Involved in Cancer Associated Fibroblast (CAF) Pro-Tumorigenic Function

Cancer associated fibroblasts (CAFs) are involved in pro-tumorigenic functions that contribute to the development of cancers, such as head and neck squamous cell carcinoma (HNSCC). To explore whether long non-coding RNAs (lncRNAs) may be involved in CAF pro-tumorigenic functions, target lncRNAs candidates that may be associated with pro-tumorigenic CAF were identified using the workflow. Deep bulk RNA-seq data of human dermal fibroblasts (HDFs) and induced CAFs were generated and analyzed by first quality evaluating raw sequencing files using FastQC program and mapping to the Genome Reference Consortium Human Build 38 (GRCh38/hg38) reference genome assembly using Spliced Transcript Alignment to a Reference (STAR) program. The sequencing files were further processed with a pipeline based on StringTie and Cufflinks tools to assemble the transcriptome and identify novel lncRNAs. Novel lncRNAs were defined to be un-annotated genes of 200 bp and >1 exon that were predicted to be non-coding. To filter the list of novel lncRNAs for lncRNA candidates, lncRNA expression quantification, using the featureCounts tool, was analyzed. In addition, protein-coding genes (PCGs) near each of the lncRNA locus was identified using BEDOPS program to identify lncRNA candidates with nearby CAF-relevant PCGs (e.g., LRRC15, COL12A1, DYRK2, FN1, CAPN9). A CAF-relevant PCG nearby the lncRNA can indicate a potential co-regulatory function. The workflow also involved analyzing the identified lncRNA candidates with public dataset, which included scRNA-seq data from HNSCC patients, bulk RNA-seq data from The Cancer Genome Atlas (TCGA)-HNSCC samples, and Encyclopedia of DNA Elements (ENCODE)/Genotype-Tissue Expression (GTEx) database, to further select lncRNA candidates of interest.

To analyze the identified lncRNA candidates from dataset with publicly available scRNA-seq data from HNSCC patients, the scRNA-seq data (Puram et al, Single-cell transcriptomic analysis of primary and metastatic tumor ecosystems in head and neck cancer.

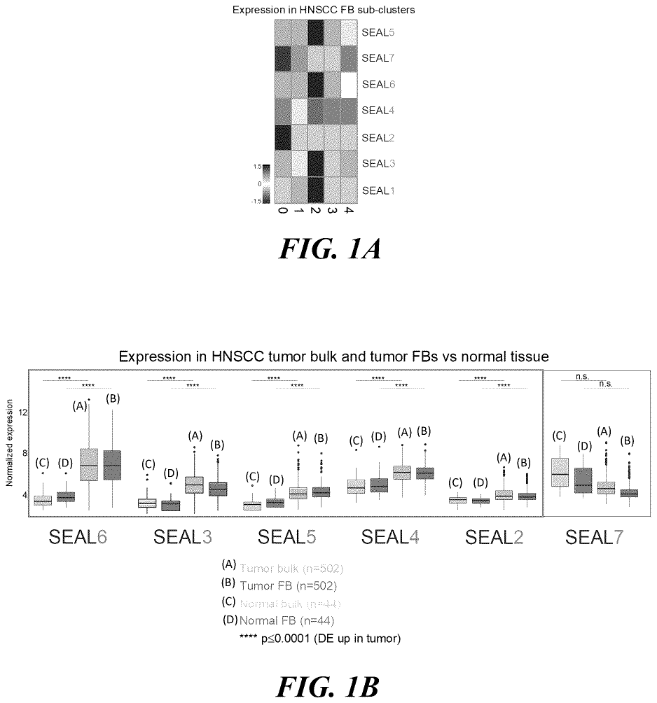

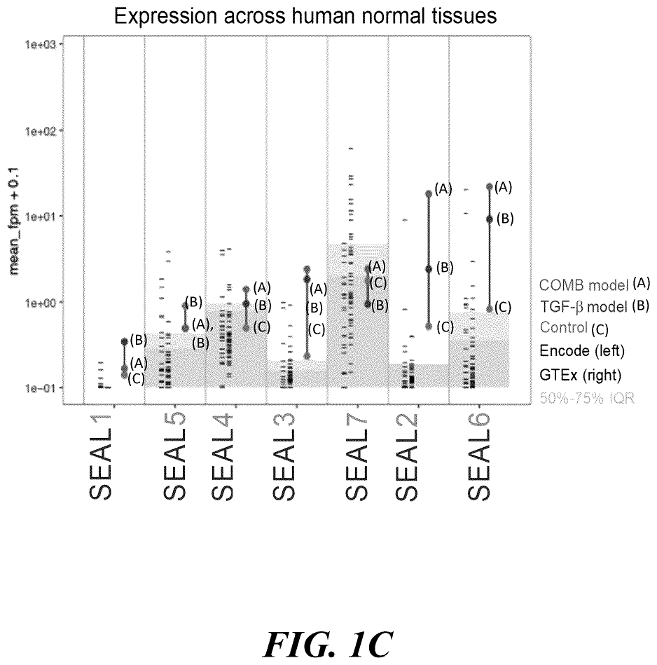

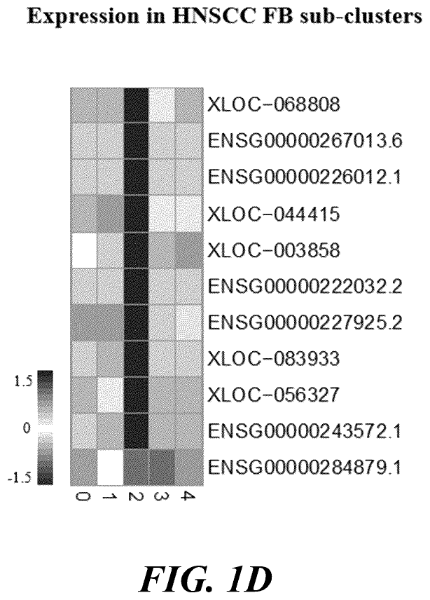

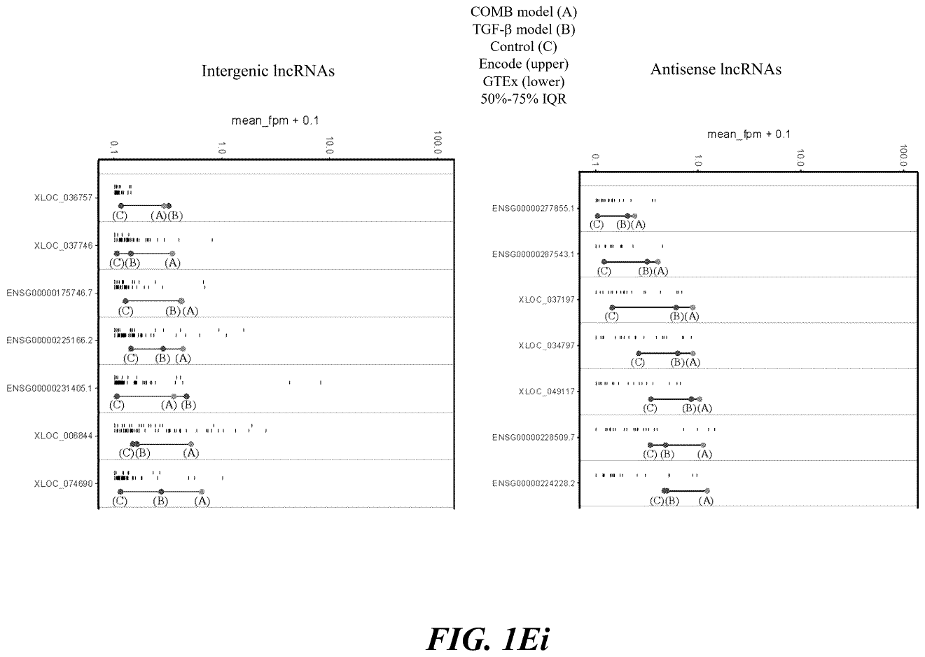

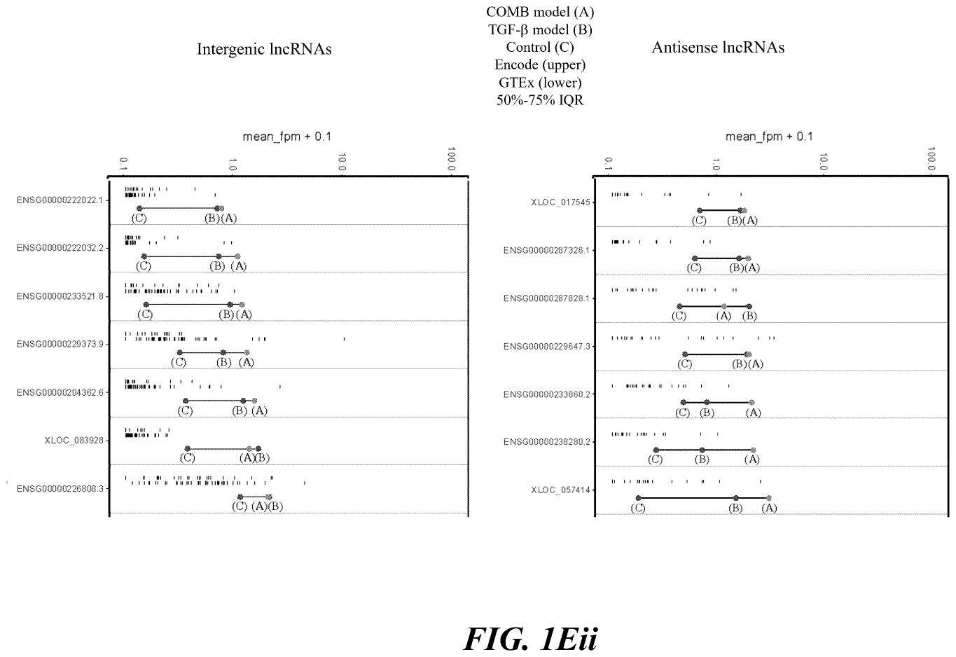

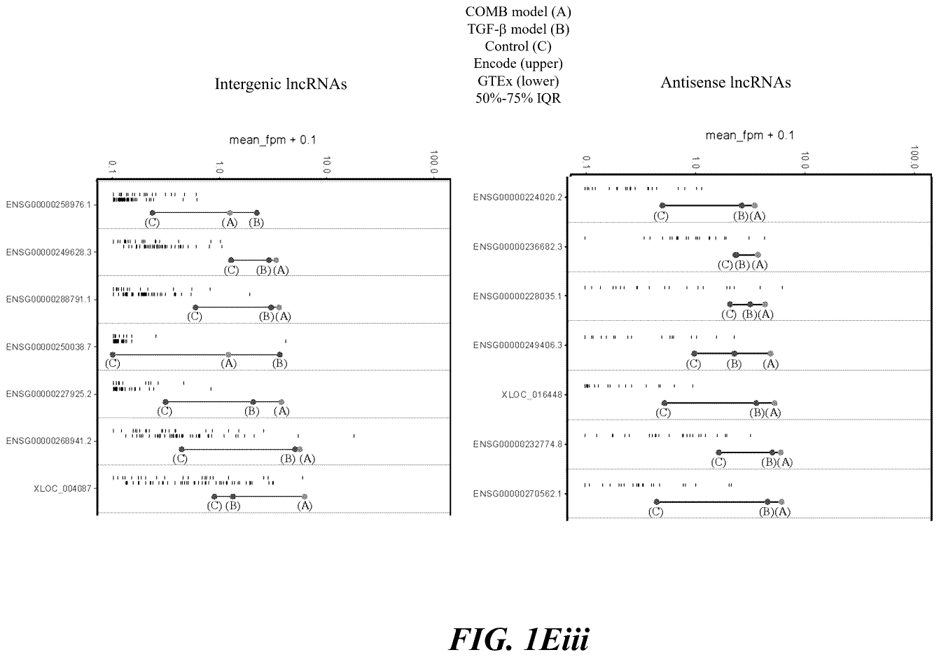

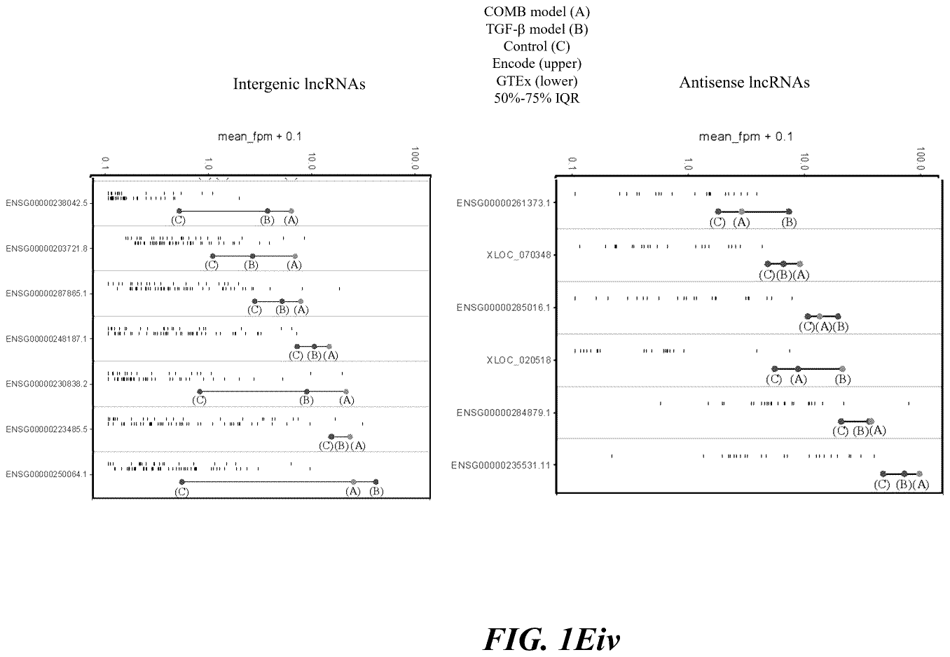

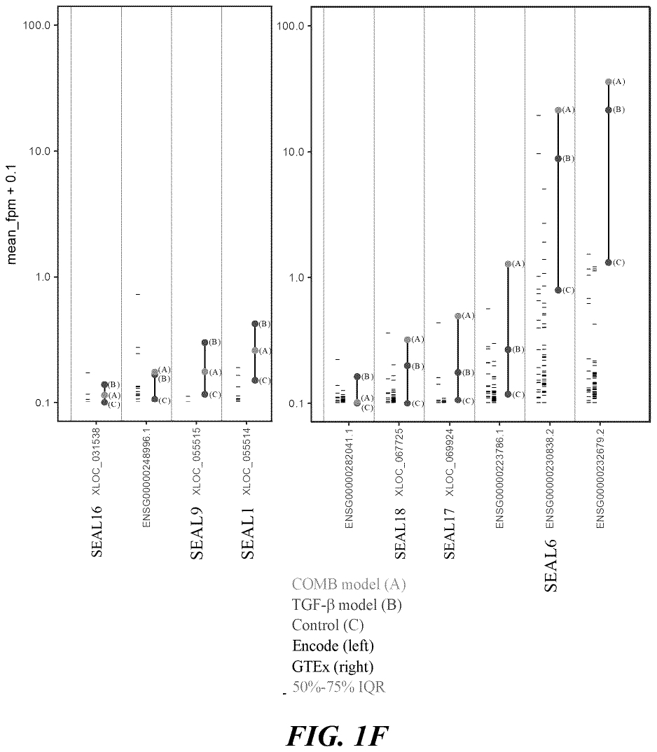

Cell 171(7), 2017) was obtained and Spearman's correlation coefficient of the global transcriptome between all pairs of cells in the raw and processed data was calculated to classify the cell as malignant versus non-malignant. Cell types were inferred to non-malignant cells with the Seurat's label transfer method (e.g., FindTransferAnchors), using the processed data as the reference and raw data as query to identify lncRNAs that were in non-malignant cells in the tumor microenvironment. Next, the fibroblasts (FBs) were sub-clustered using Seurat's method FindClusters at a resolution of 0.6 to identify lncRNAs of non-malignant fibroblasts. The FB sub-cluster identities were validated by either individual gene expression (e.g., MCAM and RGS5 as pericyte-markers) or gene set enrichment analysis of CAF-relevant marker lists, using SingScore R package. Results identified cluster 0 and 1 to be made up of pericyte-like cells. Clusters 2 and 3 were found to comprise of CAFs, with cluster 2 made up specifically of myofibroblast CAFs (myCAF)-like. Cluster 4 was identified to be made up of resident FB-like cells. For quantification of lncRNA candidates identified from dataset in the HNSCC CAF sub-clusters, the Salmon mapping-based quantification program was used to quantify gene-level expression across the cells of the HNSCC scRNA-seq dataset. The dataset was then log-normalized and scaled. As shown in the heatmap representation of the results in FIG. 1A, candidate target lncRNAs called Stromal Enhancer Associated lncRNAs (SEALs; SEAL1, SEAL2, SEAL3, SEAL4, SEAL5, SEAL6, SEAL7, see Table 10) were highly expressed in fibroblasts. In particular, heatmap representation of gene expression in the HNSCC CAF sub-clusters showed that SEAL1, SEAL3, SEAL5, and SEAL6 were specifically expressed in HNSCC FB cluster 2, representing myCAFs (FIG. 1A). Out of the 7 SEALs, SEAL1, SEAL2, and SEAL3 were not previously annotated lncRNAs. To identify further potential lncRNA candidates based on the HNSCC scRNA-seq data, lncRNAs that were differentially upregulated (based on log 2FC >1) in TGFβ and/or COMB induced CAFs and also differentially upregulated in the HNSCC cluster 2 myCAF-like FBs (based on adjusted p-value 0.05 and log 2FC >0) were extracted. Out ofthose lncRNAs, the ones showing particularly specific expression in cluster 2 FBs and low expression in the other FB clusters, were selected (FIG. 1D, Table 12). LncRNAs that were differentially upregulated in TGFβ and/or COMB induced CAFs (based on adjusted p-value≤0.05 and log 2FC >0.5) were also assessed for presence of a human colorectal cancer CAF super-enhancer (SE) region. SE regions were identified by Rank Ordering of Super-Enhancers (ROSE) algorithm using a publicly available dataset of primary colorectal cancer CAF H3K27ac ChIP-seq data (Lee et al, PRRX1 is a master transcription factor of stromal fibroblasts for myofibroblastic lineage progression. Nature Communications 19(1), 2022). LncRNAs that were differentially upregulated and associated with a SE region were assessed for tissue specificity and lncRNAs showing a higher expression in induced CAFs than in normal tissues (FIG. 1E, Table 12) were selected as candidate lncRNAs. Antisense lncRNAs were only compared to the ENCODE project data repository as those data contain strand information (antisense lncRNAs are strand-specific).

TABLE 12
Genomic regions (coordinates with strand information)
which encode IncRNAs associated with myCAF

| LncRNA name | LncRNA category | Genomic coordinates (strand) |
|---|---|---|
| XLOC_068808 | Antisense, novel | chr6: 169218159-169219142 (+) |
| ENSG00000267013.6 | Intergenic, annotated | chr18: 55105904-55124306 (−) |
| ENSG00000226012.1 | Antisense, annotated | chr21: 38237217-38238201 (−) |
| XLOC_044415 | Antisense, novel | chr2: 215373406-215380595 (+) |
| XLOC_003858 | Antisense, novel | chr1: 245799772-245813879 (+) |
| ENSG00000222032.2 | Intergenic, annotated | chr2: 237428920-237434883 (−) |
| ENSG00000227925.2 | Intergenic, annotated | chr1: 221819842-221840717 (−) |
| XLOC_083933 | Antisense, novel | chr9: 134828897-134829761 (−) |
| XLOC_056327 | Antisense, novel | chr3: 55066447-55098407 (−) |
| ENSG00000243572.1 | Intergenic, annotated | chr3: 55178172-55189525 (+) |
| ENSG00000284879.1 | Antisense, annotated | chr2: 87455476-87767359 (+) |
| ENSG00000277855.1 | Antisense, annotated | chr3: 71581721-71628694 (+) |
| ENSG00000287543.1 | Antisense, annotated | chr15: 74040190-74043332 (−) |
| XLOC_034797 | Antisense, novel | chr17: 76361430-76372793 (−) |
| XLOC_049117 | Antisense, novel | chr20: 4163106-4172518 (−) |
| ENSG00000228509.7 | Antisense, annotated | chr2: 190672297-190843734 (−) |
| ENSG00000224228.2 | Antisense, annotated | chr1: 172775905-173064474 (+) |
| XLOC_017545 | Antisense, novel | chr12: 1504740-1532192 (−) |
| ENSG00000287326.1 | Antisense, annotated | chr10: 126387092-126460957 (+) |
| ENSG00000229647.3 | Antisense, annotated | chr2: 207166120-207249105 (+) |
| ENSG00000233860.2 | Antisense, annotated | chr4: 76708853-76802426 (−) |
| ENSG00000238280.2 | Antisense, annotated | chr10: 62624249-62805887 (−) |
| XLOC_057414 | Antisense, novel | chr3: 156480878-156492990 (−) |
| ENSG00000224020.2 | Antisense, annotated | chr9: 124658412-124744618 (+) |
| ENSG00000236682.3 | Antisense, annotated | chr2: 127387077-127406228 (+) |
| ENSG00000228035.1 | Antisense, annotated | chr1: 115229423-115368072 (+) |
| ENSG00000249406.3 | Antisense, annotated | chr17: 50199876-50215922 (+) |
| XLOC_016448 | Antisense, novel | chr12: 57239370-57243163 (+) |
| ENSG00000232774.8 | Antisense, annotated | chr14: 61570405-61662070 (+) |
| ENSG00000270562.1 | Antisense, annotated | chr3: 71584943-71587409 (+) |
| XLOC_070348 | Antisense, novel | chr6: 116351101-116371719 (−) |
| XLOC_020518 | Antisense, novel | chr13: 101694567-101706175 (+) |
| ENSG00000284879.1 | Antisense, annotated | chr2: 87455476-87767359 (+) |
| ENSG00000235531.11 | Antisense, annotated | chr8: 71828167-72121231 (+) |
| XLOC_037197 | Antisense, novel | chr19: 2602781-2612054 (+) |
| ENSG00000287828.1 | Antisense, annotated | chr1: 3306636-3327974 (−) |
| ENSG00000261373.1 | Antisense, annotated | chr16: 89711856-89718165 (+) |
| ENSG00000285016.1 | Antisense, annotated | chr2: 111429324-111699033 (−) |
| XLOC_036757 | Intergenic, novel | chr18: 55786092-55840306 (−) |
| XLOC_037746 | Intergenic, novel | chr19: 18791314-18799563 (+) |
| ENSG00000175746.7 | Intergenic, annotated | chr15: 39250669-39254845 (+) |
| ENSG00000225166.2 | Intergenic, annotated | chr2: 215438221-215486639 (+) |
| XLOC_006844 | Intergenic, novel | chr1: 200326148-200337384 (−) |
| XLOC_074690 | Intergenic, novel | chr7: 1288260-1291258 (−) |
| ENSG00000222022.1 | Intergenic, annotated | chr2: 237421420-237425276 (−) |
| ENSG00000222032.2 | Intergenic, annotated | chr2: 237428920-237434883 (−) |
| ENSG00000258976.1 | Intergenic, annotated | chr14: 74614433-74616647 (−) |
| ENSG00000229373.9 | Intergenic, annotated | chr13: 113888222-113923512 (+) |
| XLOC_083928 | Intergenic, novel | chr9: 134622841-134625269 (−) |
| ENSG00000204362.6 | Intergenic, annotated | chr1: 17185833-17198177 (+) |
| ENSG00000226808.3 | Intergenic, annotated | chr10: 43778946-43901004 (+) |
| ENSG00000249628.3 | Intergenic, annotated | chr12: 1500491-1510070 (+) |
| ENSG00000288791.1 | Intergenic, annotated | chr2: 215758890-215777870 (+) |
| ENSG00000227925.2 | Intergenic, annotated | chr1: 221819842-221840717 (−) |
| XLOC_004087 | Intergenic, novel | chr1: 3482551-3487051 (−) |
| ENSG00000238042.5 | Intergenic, annotated | chr1: 221812364-221978523 (−) |
| ENSG00000203721.8 | Intergenic, annotated | chr1: 200253419-200400705 (−) |
| ENSG00000287865.1 | Intergenic, annotated | chr7: 155213744-155242723 (+) |
| ENSG00000248187.1 | Intergenic, annotated | chr4: 128563770-128570567 (−) |
| ENSG00000230838.2 | Intergenic, annotated | chr2: 215718043-215720946 (+) |
| ENSG00000223485.5 | Intergenic, annotated | chr6: 169151349-169163007 (−) |
| ENSG00000250064.1 | Intergenic, annotated | chr4: 28117558-28638131 (+) |
| ENSG00000231405.1 | Intergenic, annotated | chr22: 27138352-27159878 (+) |
| ENSG00000233521.8 | Intergenic, annotated | chr22: 27220842-27225134 (−) |
| ENSG00000250038.7 | Intergenic, annotated | chr4: 28315671-28402936 (−) |
| ENSG00000268941.2 | Intergenic, annotated | chr20: 58622930-58635835 (+) |
| ENSG00000248996.1 | Antisense, annotated | chr5: 177494995-177503647 (+) |
| XLOC_031538 (SEAL16) | Antisense, novel | chr17: 13541892-13545884 (+) |
| XLOC_067725 (SEAL18) | Intergenic, novel | chr6: 74872099-74902546 (+) |
| ENSG00000282041.1 | Intergenic, annotated | chr22: 35376422-35377261 (+) |
| XLOC_069924 (SEAL17) | Intergenic, novel | chr6: 75030717-75046445 (−) |
| ENSG00000223786.1 | Intergenic, annotated | chr6: 74069451-74690727 (+) |
| ENSG00000232679.2 | Intergenic, annotated | chr1: 221996310-222064797 (−) |
| XLOC_075178 (SEAL14) | | chr7: 40706463-40711285 (−) |
| XLOC-054623 (SEAL15) | | chr3: 124845800-124846379 (+) |
| XLOC_049867 (SEAL19) | | chr20: 57985899-57999562 (−) |

LncRNA candidates identified from dataset were analyzed with another publicly available scRNA-seq data from HNSCC patients that includes patient samples from different stages of tumor progression (Choi et al, Single-cell transcriptome profiling of the stepwise progression of head and neck cancer. Nature Communications 14(1), 2023). Seurat package was used for preprocessing analysis. Seurat Object was created by including cells that have at least 100 genes detected, and genes expressed in at least 3 cells. RunH-armony integration was then performed to adjust for ample condition batch effects (parameters theta=0, lambda1, sigma=0.1), followed by UMAP dimensionality reduction (dims=1:50). Seurat's method FindClusters was used to first identify the cell types and then the Fibroblast (FB) sub-clusters. The expression of FB markers, DCN, LUM, COL1A1, COL1A2, COLUA1, and COL6A2, was confirmed to be predominantly expressed by the FB cluster, wherein epithelial cells were recognized by their expression of epithelial cell markers (e.g., KRT17, KRT6A, KRT5, KRT19, KRT8, KRT16, KIRT18, KIRT6B, KRT15, KRT6C, KARTCAP3, SFN, and EPCAM) and distinct from the FB cluster. FB sub-clusters 2 and 3 were annotated to be myCAF-like based on gene set enrichment analysis of a myCAF marker list using the SingScore R package. FBs of each sample condition (normal tissue, pre-cancerous leukoplakia and primary cancer) were then analyzed. The majority of normal tissue FBs were composed of sub-clusters 0, 1 and 4, leukoplakia FBs partially of sub-cluster 0 but mainly of sub-cluster 2, and primary cancer partially of sub-cluster 2 but mainly of sub-cluster 3. Based on this analysis, the sub-cluster 2 FBs were defined as partially transitioned myCAFs and sub-cluster 3 as fully differentiated myCAFs. To define a myCAF signature which is specific for the fully differentiated myCAF state, the protein-coding genes (PCGs) differentially upregulated in primary cancer FBs sub-cluster 2+3, as compared to normal tissue FBs sub-clusters 0+1+4 were extracted. The primary cancer FBs upregulated PCGs were then intersected with the myCAF marker list (Buechler et al, Cross-tissue organization of the fibroblast lineage. Nature 593(7860), 2023) used above, giving 198 PCGs in common between the two lists. These 198 PCGs constitute a defined myCAF signature that was used for subsequent analyses (Table 13). This defined myCAF signature was confirmed to not be expressed in normal tissue FBs, to be partially expressed in leukoplakia FBs and particularly enriched in primary cancer FBs, as assessed using the SingScore R package. Further, the scRNA-seq data from HNSCC patients that includes patient samples from different stages of tumor progression was used for deconvolution of the TCGA-HNSC bulk RNA-seq data using BayesPrism (Chu et al, Cell type and gene expression deconvolution with BayesPrism enables Bayesian integrative analysis across bulk and single-cell RNA sequencing in oncology. Nat Cancer 3(4), 2022). By this analysis, the TCGA-HNSC patient (n=499) cohort was partitioned based on HNSCC Cluster 3 nyCAF fraction, identifying myCAF fraction—High (n=145) vs nyCAF fraction—Low (n=354) patient groups. Kaplan-Meier survival analysis based on Overall Survival (OS) was then performed, showing that the myCAF fraction—High patient category was associated with significantly (p=0.0074) worse OS compared to the myCAF fraction—Low patient category, with a median survival of 2.5 years as compared to 4.5 years, respectively (FIG. 21A). This finding was validated in another independent HNSCC sc-RNA-seq dataset. This suggested that the myCAF signature marks a cell population of prognostic value in a large patient cohort, further emphasizing this signature as a relevant read-out for unfavorable myCAF disease-states.

Next, lncRNAs were identified by deep RNA-sequencing of HDFs and induced CAFs (e.g., 150 million reads/sample). Specifically, an in-house reconstructed gtf of controlled or induced CAFs was created, containing information about 76685 genes. Out of these 76685 genes, 46335 genes were identified to encode annotated mRNAs (n=20006, 43%), annotated lncRNAs (n=17568, 38%) or novel lncRNAs (n=8761, 19%). Novel lncRNA genes were defined as previously un-annotated genes encoding transcripts of more than 200 bp, with more than one exon, and of predicted non-coding potential. While these three gene categories represent around 61% of the total number of genes, they encode for around 90% (n=331332 mRNA, n=74230 annotated lncRNA and n=13236 novel lncRNA transcripts) of the total number (467369) of transcripts, thereby constituting the majority of the transcriptome. Other gene categories such as novel mRNA genes, pseudogenes or other non-coding RNA genes were therefore not considered in the current study. These gene categories were further evaluated for their expression across human normal tissues of the ENCODE repository. Annotated mRNA genes did not show tissue specific expression while, in contrary, lncRNA genes displayed higher tissue specificity. Novel lncRNA genes surpassed the other gene categories with a majority showing high tissue specificity.

The identified lncRNAs were selected based on having overlapping (antisense lncRNAs), or neighboring (intergenic lncRNAs), loci with PCGs of the defined myCAF signature. They were further selected based on having a positive fold change (log 2FC) expression induction in induced CAFs, as compared to HDFs. The top 10 antisense lncRNAs identified were SEAL14, ITPRIP-AS1, SEAL15, P4HA3-AS1, SEAL16, PDLIM7-AS1, SEAL9, SEAL1, and NKILA. The top 10 intergenic lncRNAs identified were LINC03004, LINC02093, ENSG0000282041, SEAL17, SEAL18, ENSG00000287335, ENSG00000223786, SEAL19, SEAL6, and LINC01705.

Some of these lncRNAs were found to be expressed in fibroblasts of HNSCC, with a subset of the lncRNAs associated with a specific fibroblast sub-cluster. The top 10, based on log 2FC expression, antisense and intergenic lncRNAs of this category were evaluated for tissue specificity using the ENCODE project and GTEx portal data repositories as described. For antisense lncRNAs, only ENCODE project data was analyzed as that data is strand-specific (strand-specific data is required to accurately quantify antisense lncRNA expression). As shown in FIG. 1F, 4 out of the 10 antisense lncRNAs (including SEAL1 and SEAL9) and 6 out of the 10 intergenic lncRNAs (including SEAL6) showed higher expression in induced CAFs than the inter-quartile range (IQR) expression across human normal tissues (Table 12).

TABLE 13
Additional myCAF marker signature genes
Gene name

ACTA2, ACTN1, ADAM12, ADAMTS12, AEBP1, ALDH18A1, ANTXR1, ARF4, ARL4C, BACE2, BASP1,
BGN, BHLHE40, BMP1, BST2, C11orf24, C1orf198, C1QTNF6, CADM1, CALD1, CALU, CCND1, CD276,
CDC42EP3, CERCAM, CHN1, CHPF, CKAP4, CLEC11A, CLIC4, CNN2, COL10A1, COL11A1, COL12A1,
COL1A1, COL1A2, COL5A2, COL8A1, COLGALT1, CREB3L1, CSRP2, CTHRC1, CTSB, CTSK, CTSZ,
CXCL2, CXCL3, DAP, DIO2, DPYSL3, DUSP10, EDIL3, EDNRA, EFEMP2, EGFL6, ERN1, FAP, FBXO32,
FKBP10, FN1, FSCN1, FZD1, GAPDH, GEM, GGT5, GJA1, GLT8D2, GOLM1, GPX7, GPX8, GREM1,
HAPLN3, HCFC1R1, HES4, HLA-B, HLA-C, HS3ST3A1, ID1, ID4, IER3, IFI27, IFI6, IL32, INHBA, ITGB5,
ITPRIP, KDELR2, KDELR3, KIAA1217, KIF26B, KLF6, LAMP5, LEF1, LMCD1, LMO7, LOXL2, LRRC15,
LUM, MAGED1, MARCKSL1, MARVELD1, MDK, MICAL2, MIF, MMP11, MMP14, MMP19, MMP2,
MSRB3, MXRA5, MYH9, MYL9, NEK6, NR4A2, NREP, NRP2, NTM, NXN, OLFML2B, P3H1, P3H4,
P4HA3, P4HB, PALLD, PARVA, PDGFC, PDLIM7, PEA15, PERP, PKM, PLOD1, PLOD2, PMAIP1,
PMEPA1, PODNL1, POSTN, PRDX4, PRSS23, PTGER3, PTK7, PYCR1, RAB31, RAI14, RBM3, RCAN2,
RCN1, RCN3, RGCC, RGS3, RIN2, RNF144A, ROR2, RUNX2, SCARF2, SDC1, SEC13, SERPINH1, SFRP2,
SHISA5, SLC16A3, SLC38A5, SLC39A14, SMCO4, SMIM3, SMYD3, SNAI2, SPARC, SPATS2L, SPHK1,
SPON1, SSR3, STK17B, SUGCT, SULF1, SULF2, SYTL2, TAGLN, TENM3, TGFB1I1, TGFBI, THBS2,
THY1, TMEM119, TMEM263, TMEM45A, TNFAIP3, TOM1, TPM1, TPM4, TPST2, TSPO, TUBA1C,
TUSC3, UBTD1, UNC5B, VCAN, VGLL4, VOPP1

Furthermore, bulk RNA-seq of TCGA HNSCC samples were deconvoluted by using the HNSCC scRNA-seq data and applied the Bayesprim R package to investigate cell type-specific gene expression. The output of Bayesprism analysis is a cell type-specific gene expression count matrix from which fibroblast specific expression can be extracted. As shown in FIG. 1B, the expression of lncRNA candidates SEAL2, SEAL3, SEAL4, SEAL5, and SEAL6 were upregulated in the tumor fibroblast cell population compared to normal tissue fibroblasts, suggesting the possible role of the lncRNA candidates in tumor fibroblast cell populations.

For tissue specificity evaluation, the expression of lncRNA candidates was evaluated with the ENCODE project and GTEx portal data repositories of normal tissue types bulk RNA-seq. Gene expression was quantified using the tool featureCounts and normalized using the R package DESeq2. As shown in FIG. 1C, the expression of lncRNA candidates SEAL1, SEAL2, SEAL3, and SEAL6 in the induced CAFs was higher than the mean expression and the inter-quartile range (IQR) expression across the normal human tissues. This data suggested that lncRNA candidates SEAL1, SEAL2, SEAL3 and SEAL6 have low expression across normal tissues. Collectively, these analyses revealed SEAL1, SEAL9, SEAL2, SEAL3, SEAL4, SEAL5, SEAL6, and SEAL7 to be possible lncRNA candidates for further exploration of their involvement in CAFs.

### Example 2: Generation of TGF, and TGFβ+Starvation (COMB) Induced CAF Models

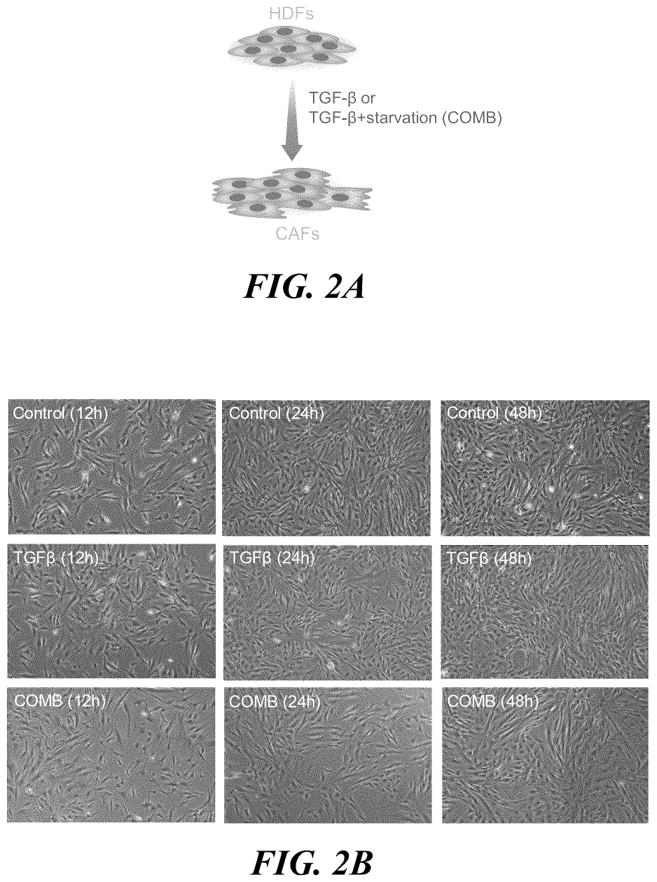

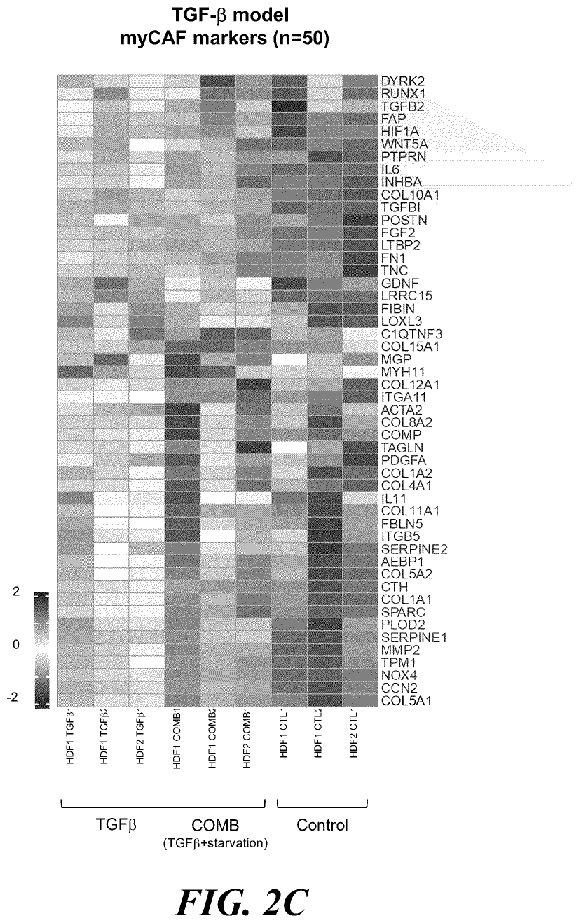

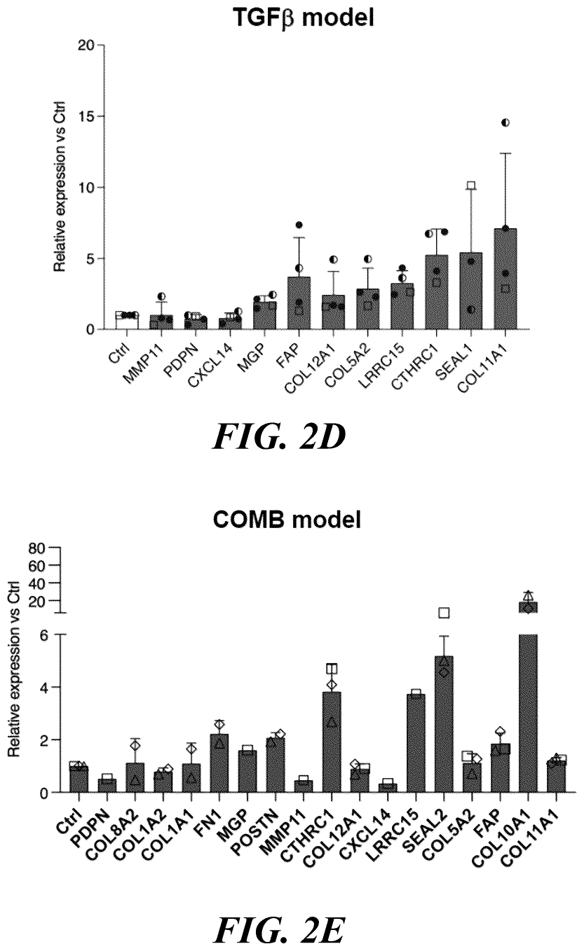

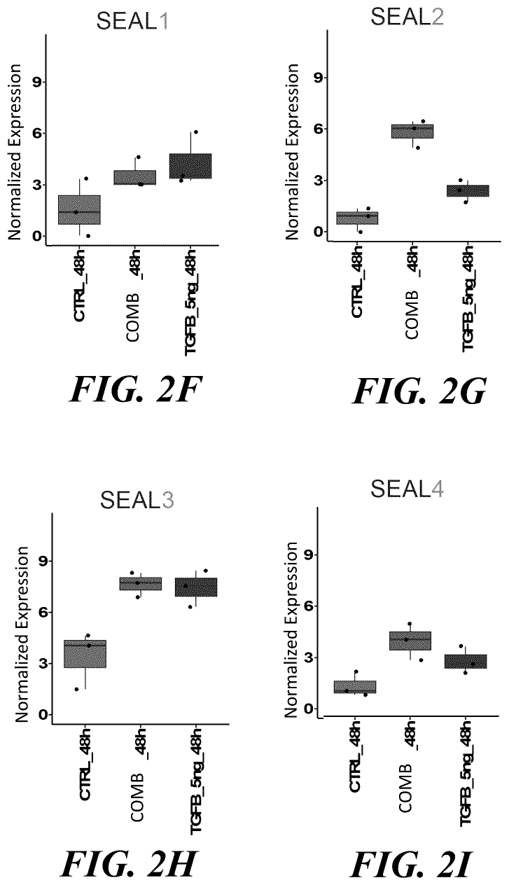

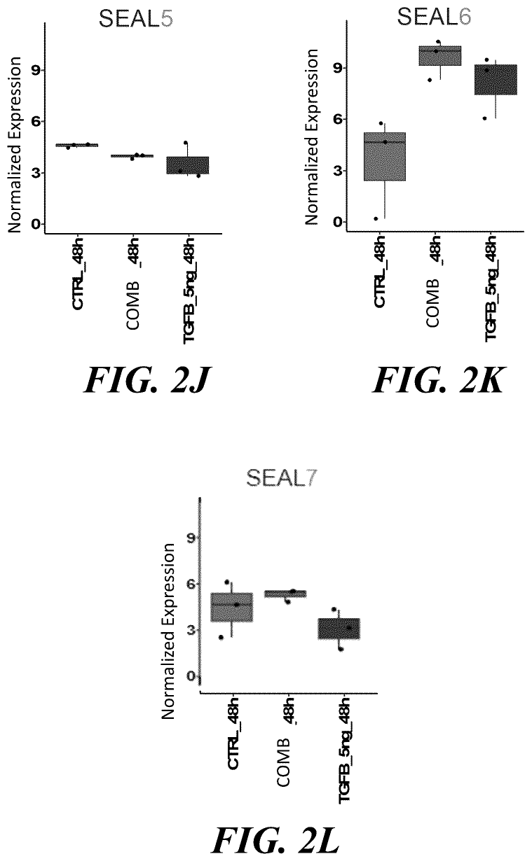

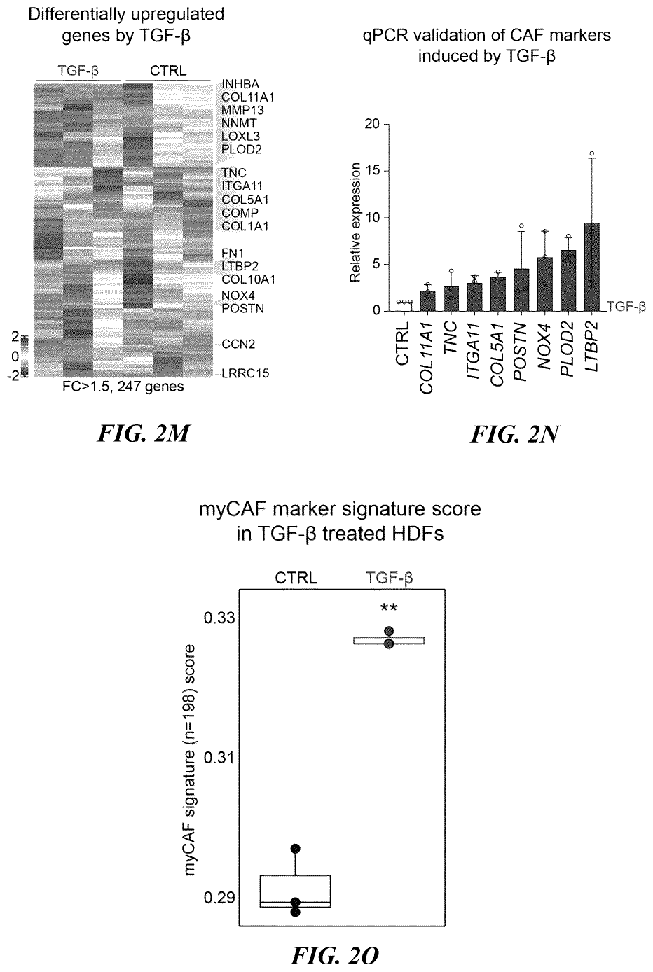

To characterize and investigate the function of the identified lncRNA candidates (e.g., SEAL1, SEAL9, SEAL2, SEAL3, SEAL4, SEAL5, SEAL6, SEAL7), as described in Example 1, an in vitro myofibroblast CAF (myCAF) model was established, as shown in FIG. 2A. Primary HDFs from healthy donors were obtained and cultured at 70-90% confluency in FGM-2 Fibroblast Growth Medium-2 BulletKit (Lonza) and were passaged 1-2 times/week using TrypLE Express (Gibco). All experiments were performed on low-passage HDFs (less than passage 5). For TGFβ and TGFβ+ starvation (COMB) induction, 100,000 HDFs were seeded in 6-well plates and were left to acclimate for 48 hours before induction. For TGFβ induction, cells were treated with 5 ng/ml TGFβ for 48 hours and then harvested for downstream analyses. For COMB induction, cells were treated with 5 ng/ml TGFβ in cell medium without fetal bovine serum (FBS) (starvation) for 48 hours before harvest. As shown in FIG. 2B, HDFs without induction, and with TGFβ or COMB induction were monitored at 12 hours, 24 hours, and 48 hours, and the fibroblasts were still viable with TGFβ or COMB induction.

Once a TGFβ/COMB induced model was established, gene expression of TGFβ/COMB induced CAFs was investigated by performing RNA-seq. RNAs (e.g., 500 ng) were extracted from control HDFs and TGFβ/COMB induced CAFs. With the extracted RNAs, polyA-enriched or Ribozero-depleted strand-specific libraries were prepared and sequenced on the Illumina NovaSeq 6000 PE150 at a depth of 40-200 million reads/sample. RNA-seq data were analyzed for differential gene expression. As shown in FIG. 2C, expression of several myofibroblast CAF (myCAF) gene markers, such as COL10A1, COL11A1, COL5A2, FAP, were upregulated compared to control, suggesting that the TGFβ/COMB was able to induce HDFs to myofibroblast CAFs. This finding was validated with RNAs from HDFs and TGFβ/COMB induced CAFs, reverse transcribed using the QuantiTect Reverse transcription kit (Qiagen), and subjecting the reverse transcribed RNAs to qPCR analysis. qPCR analysis was performed with TB Green Premix Ex Taq II (Tli RNase H Plus) (Takara) and specific TaqMan assays for myCAF gene markers, and control gene RPL30 to normalize expression values to account for differences in cDNA input. As shown in FIG. 2D and FIG. 2E, qPCR analysis showed that some expression of myCAF gene markers (e.g., COL11A1, COL5A1, LRRC15) are upregulated in TGFβ and COMB induced CAFs compared to control, respectively. As shown in FIG. 2M, expression of several myCAF gene markers, such as INHBA, COL11A1, MMP13, NNMT, LOXL3, PLOD2, TNC, ITGA11, COL5A1, COMP, COL1A1, FN1, LTBP2, COL10A1, NOX4, POSTN, CCN2 and LRRC15 were upregulated compared to control, suggesting that the TGFβ model was able to induce HDFs to myofibroblast CAFs. The upregulation of these markers in TGFβ induced CAFs, as compared to control, was validated by performing qPCR analysis of the myCAF gene markers COL11A1, TNC, ITGA11, COL5A1, POSTN, NOX4, PLOD2 and LTBP2, as shown in FIG. 2N. Singscore analysis with the myCAF signature (Table 13) confirmed that the expression of these genes was significantly enriched in TGFβ induced CAFs as compared to control HDFs, as shown in FIG. 2O. In addition, gene ontology analysis of upregulated protein-coding genes upon TGFβ induction were found to be associated with myCAF functions (e.g., extracellular matrix organization, mesenchyme development, axon guidance, and wound healing, muscle tissue development, connective tissue development, respiratory system development, bone development, ser family amino acid biosynthetic process, transmembrane receptor protein Ser/Thr kinase signaling pathway). Extracellular matrix organization was the top enriched (adjusted p-value of 1.36e−11) GO process. Of note, an enrichment of innervation processes (e.g. axon guidance pathways) and/or ECM dysregulation pathways are associated with aggressiveness, perineural invasion and outcome for some solid cancers and represent important CAF-driven pro-tumorigenic processes. Therefore, the GO pathway analysis further supported the in vitro model recapitulated the role of CAFs in tumor development.

Expression of lncRNA candidates were also analyzed with the RNA-seq data. As shown in FIG. 2F-2L, expression of SEAL1, SEAL2, SEAL3, SEAL4, SEAL6, and SEAL7 were upregulated in TGFβ and/or COMB induced CAFs compared to control, suggesting that the lncRNA candidates may modulate myCAFs.

Furthermore, snATAC-seq and snRNA-seq were performed with scramble control ASO (scrl ASO) transfected HDFs and in TGFβ induced CAFs. Briefly, 3 million of Scrl ASO transfected CTRL HDFs and TGF-β induced myCAFs were washed with PBS (Thermo Fisher), incubated in Swelling Buffer (10 mM Tris-HCl pH 7.5, 2 mM MgCl2, 3 mM CaCl₂)) for 5 min on ice, and then centrifuged at 700 g for 6 min at 4° C. Cells were resuspended in Lysis Buffer (10% v/v glycerol, 0.5% v/v NP40, 10 mM Tris-HCl pH 7.5, 2 mM MgCl2, 3 mM CaCl₂), 4 U/mL SUPERase (Thermo Fisher)), incubated for 8 min on ice, and then centrifuged at 700 g for 6 min at 4° C. Nuclei were resuspended in diluted Nuclei buffer (10× Genomics), targeting a recovery of 10,000 nuclei. Transposition, GEM generation, barcoding and sequencing library preparation was performed following the following manufacturer's instructions (Chromium Next GEM Single Cell ATAC Kit v2, PN-1000390, 10× Genomics). Libraries were sequenced on an Illumina NovaSeq S4, PE10×ATAC at a depth of 300 million reads per sample. For snRNA-seq, single nuclei preparations were barcoded using the 10× chromium platform (Single cell 3′ Reagent Kit v3, 10× Genomics). Post GEM-RT clean-up was performed on barcoded cDNA. 250 million reads/sample were sequenced on a NovaSeq 6000 (Illumina). snRNA-seq raw data was mapped to the GRCh38/hg38 reference genome assembly and processed using 10× Genomics CellRanger (v7.1.0).

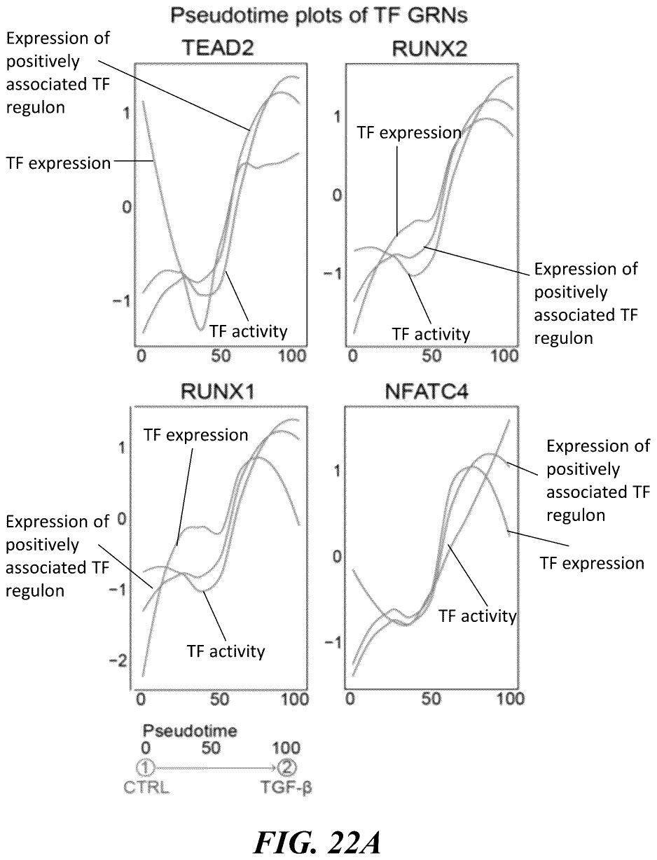

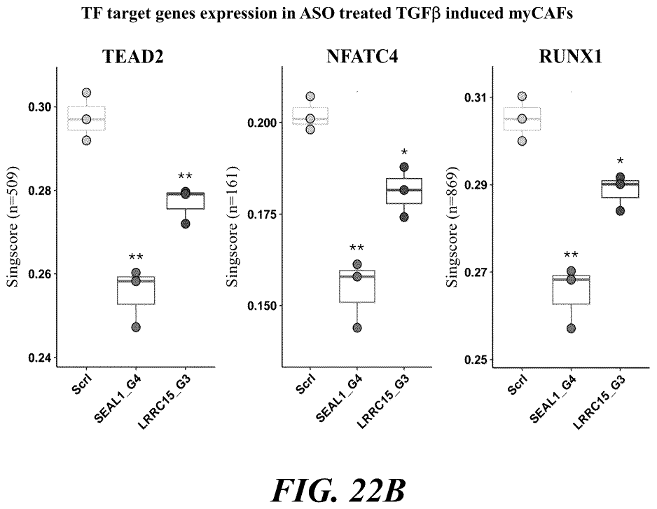

Next, the snRNA-seq and snATAC-seq data were integrated to build gene regulatory networks (GRNs) using the R package scMEGA v1.0.1 (Li et al, scMEGA: Single-cell multi-omic enhancer-based gene regulatory network inference. Bioinformatics Advances 3(1), 2023). The integrated, pair snRNA/ATAC dataset was subjected to trajectory analysis to identify cell states. Identified transcription factors (TFs) were filtered based on correlation between TF binding activity (chromVAR) and TF expression along trajectories, and PCGs based on correlation between chromatin accessibility (ATAC signal) and expression along trajectories. This analysis identified 47 TFs associated with the TGF-β induced myCAF cell state, as measured by increased activity and expression. The 47 TFs include SREBF1, IKZF1, PITX1, XBP1, VDR, ZNF274, HOXA2, RELA, MZF1, PRRX2, RUNX3, TBX5, ZSCAN29, ZIC4, NR2C2, SPI1, ATF3, SOX4, GMEB2, ZNF740, HSF1, ZNF16, FIGLA, GATA6, ETV5, BCL6, KLF17, NFATC1, TEAD2, ATF4, GLIS3, SRF, RUNX1, TEAD2, GLI2, ZNF148, TBX15, LEF1, JUND, MYC, BACH2, FOX, RUNX2, SP2, EGR2, GLIS2, AND NFATC4. Some of these TFs, such as TEAD2, NFATC4, and RUNX1, was found to be expressed in HNSCC cancer progression model (described in Example 1), demonstrating the primary cancer myCAF specific activity of these TFs. As shown in the pseudotime plots of FIG. 22A, the TFs, such as TEAD2, RUNX2, RUNX1, and NFATC4 were also found to have increase in their expression, activity, and expression of positively associated TF regulon during the transition from HDFs to TGFβ induced CAFs. In particular, the TF activity and TF regulon expression displayed a clear correlation along the pseudo-time trajectory, while the TF expression showed a less clear correlation.

### Example 3: Characterization of SEAL1 in TGFβ Induced Cancer Associated Fibroblasts (CAFs)

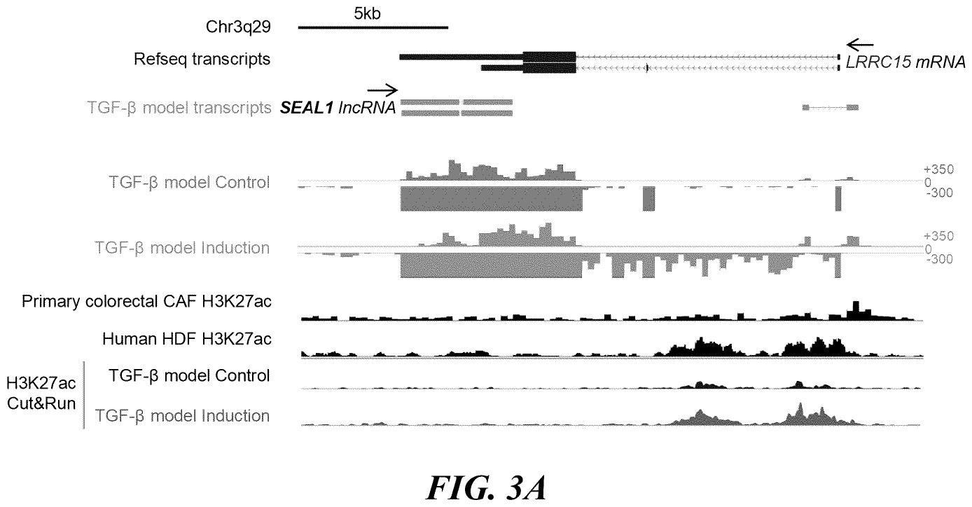

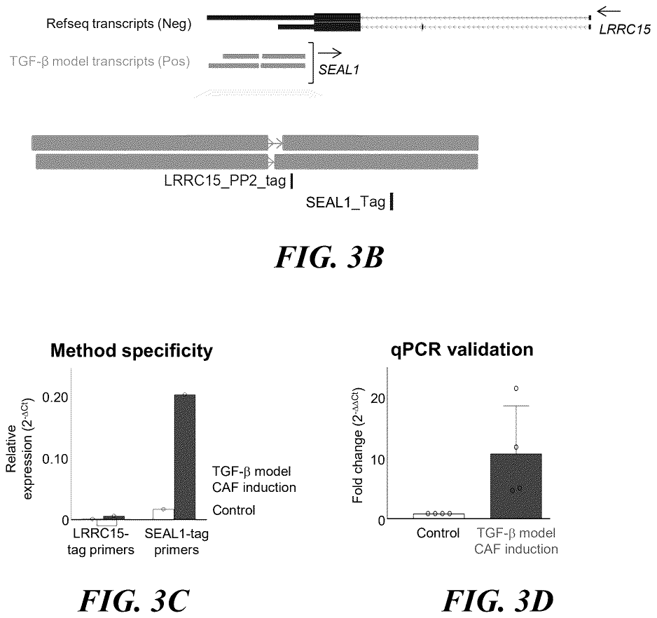

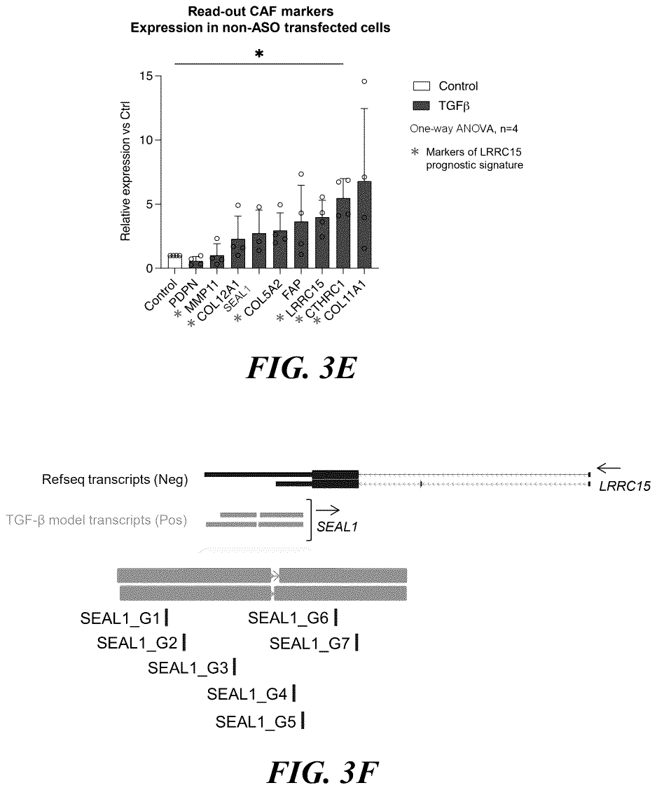

To gain insight into the genomic SEAL1 locus, bam files from the bulk RNA-seq data of TGFβ induced CAFs were used to create coverage tracks using the BAMscale tool. The output BigWig files were averaged into Wig file tracks using Wiggletools, and finally bedGraphs files (positive and negative tracks) were created for visualization in the UCSC Genome Browser on GRCh38/hg38. As shown in FIG. 3A, SEAL1 is located on Chr3:194355288-194370349 on the sense strand, overlapping with some region of LRRC15 mRNA, transcribed in the opposite (anti-sense) orientation. Expression of SEAL1 was also upregulated in TGFβ induced CAFs compared to control. In addition, since H3K27ac is associated with gene activation, human colorectal cancer CAF and HDF H3K27ac ChIP-seq data were obtained and aligned to the GRCh38 genome using Bowtie2 program to observe H3K27ac marks in the SEAL1 region (FIG. 3A, bottom). Control HDFs and TGFβ induced CAFs were further analyzed by H3K27ac Cut&Run to detect active enhancer regions. For this, HDFs were cultured and treated as described in Example 2 and 48 hours later, 500,000 cells were used for EpiCypher CUTANA™ ChIC/CUT&RUN kit with H3K27ac antibodies. Libraries were prepared using the CUTANA™ CUT&RUN library prep kit and sequenced on a NovaSeq PE100 (Illumina) at 15 million reads depth. As shown in FIG. 3A, SEAL1 was linked to a H3K27ac marked genomic region (FIG. 3A, bottom).

As SEAL1 transcript is at least partially align with some region of the LRRC15 transcript, SEAL1-specific tagged primers and LRRC15-specific tagged primers were designed, as shown in FIG. 3B. The SEAL1 transcript was detected and distinguished from the LRRC15 transcript, using a targeted cDNA-qPCR approach. First, cDNA from control and TGFβ induced CAFs was synthesized using SEAL1 specific tagged primers. Next, qPCR amplification was conducted using the tag-specific primers (e.g., LRRC15-tag primers, SEAL1 tag primers). The amplification was normalized to a reference gene (e.g., GAPDH) that was amplified using the same approach with tagged primers. As shown in FIG. 3C, SEAL1-tag primers were able to amplify specifically SEAL1, compared to LRRC15-tag primers in both control and TGFβ induced CAFs, showing the specificity of the SEAL1-tagged primers to amplify and detect SEAL1. Using the SEAL1 primers, the increased expression of SEAL1 was detected in TGFβ induced CAFs compared to control, via qPCR, as shown in FIG. 3D.

To modulate SEAL1 expression in TGFβ induced CAFs and investigate the effect of SEAL1 knockdown, various antisense oligonucleotides (ASOs) were designed to target different regions of SEAL1 transcript as shown in FIG. 3F. Prior to transfecting the cells with the ASOs, expression levels of myCAF marker transcripts and SEAL1 transcript were measured in control and TGFβ induced CAFs via qPCR to establish baseline expression of these markers without ASO treatment, as shown in FIG. 3E. FIG. 3E also shows that some of the myCAF markers that were measured are part of the LRRC15 prognostic signature. The LRRC15 prognostic signature includes MMP11, C1QTNF3, COL11A1, CTHRC1, COL12A1, COL10A1, COL5A2, THBS2, AEBP1, LRRC15, and ITGA11, which are associated with poor patient outcome across cancer types. Next, primary HDFs were induced by TGFβ at the same time as transfection with ASOs. Briefly, 100,000 primary HDFs per well of 6-well plate were plated one day before transfection with SEAL1 ASOs or Scramble control ASO (Scrl). Right before ASO transfection, the cell medium was changed to medium containing 5 ng/ml TGFβ. ASOs were diluted in TE buffer to a working solution of 20 M and was mixed with OptiMEM medium and X-tremeGENE HP transfection reagent. The mixture was incubated at room temperature for minimum of 15 minutes, and then added dropwise to the cells at 100 l/well, with the total amount of 50 pmol ASO/well. Cells were harvested after 48 hours for RNA extraction and qPCR analysis. As shown in FIG. 4A-4I, qPCR was performed to investigate expression of SEAL1 and myCAF markers (LRRC15, COL5A2, COL11A1, COL12A1, CTHRC1, MMP11, PDPN, and FAP) upon transfection of SEAL1 ASOs versus Scrl. qPCR analysis showed that transfection of SEAL1_G2, SEAL1_G4, and SEAL1_G5 ASOs clearly reduced expression of SEAL1 compared to Scrl (FIG. 4A). Transfection of SEAL1_G2 ASO also resulted in statistically significant reduced expression of COL5A2 (FIG. 4C), COL12A1 (FIG. 4E), MMP11 (FIG. 4G), and PDPN (FIG. 4H). Transfection of SEAL1_G4 ASO resulted in statistically significant reduced expression of LRRC15 (FIG. 4B), COL5A2 (FIG. 4C), COL11A1 (FIG. 4D), FAP (FIG. 4I), and CTHRC1 (FIG. 4F). Similarly, transfection of SEAL1_G5 ASO resulted in statistically significant reduced expression of LRRC15 (FIG. 4B), COL5A2 (FIG. 4C), COL11A1 (FIG. 4D), COL12A1 (FIG. 4E), CTHRC1 (FIG. 4F), and PDPN (FIG. 4H). Furthermore, LRRC15 protein level after treatment of one of the SEAL1 ASOs and an LRRC15 ASO was investigated. As shown in FIG. 4J and FIG. 4K, LRRC15 protein level increased in response to TGFβ treatment, and the LRRC15 ASO successfully eliminated LRRC15 protein expression. Interestingly, the SEAL1 ASO achieved only a partially reduced LRRC15 protein level. These data showed that downregulation of SEAL1 expression by ASOs reduces the expression of myCAF markers in the presence of some retained LRRC15 protein, suggesting that SEAL1 may achieve this effect independently from LRRC15.

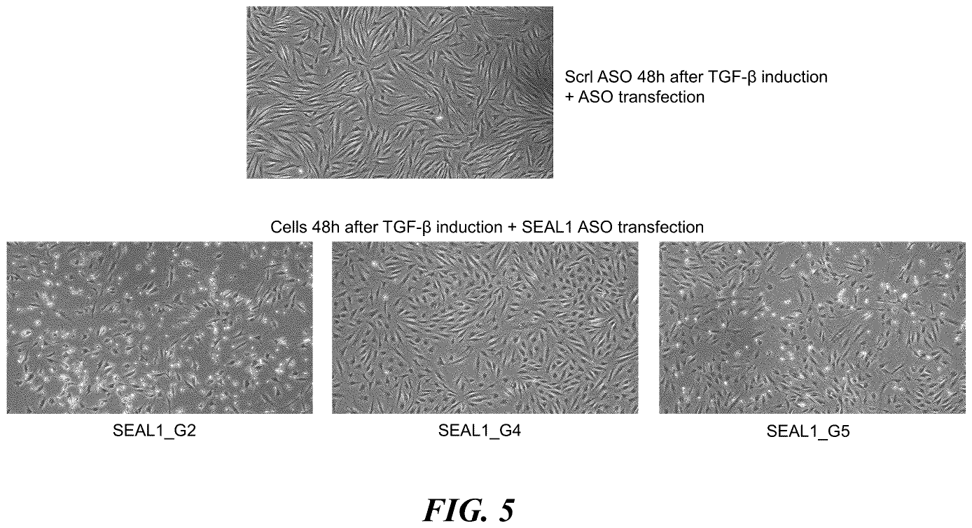

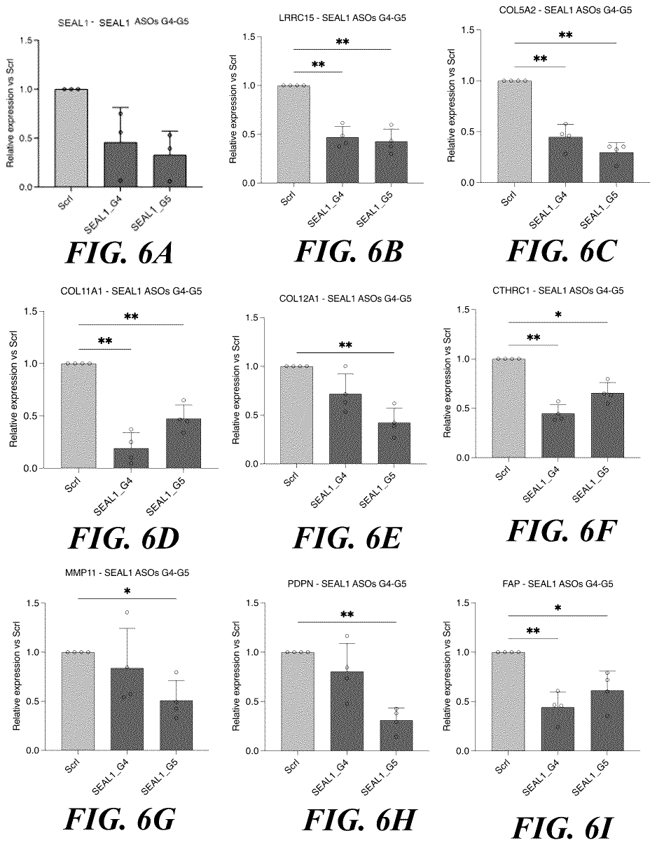

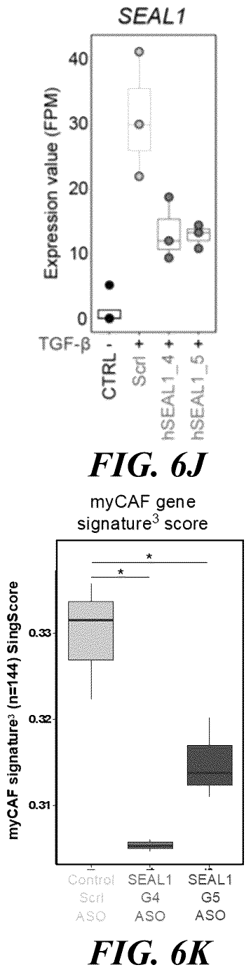

As transfection of SEAL1_G2, SEAL1_G4, and SEAL1_G5 ASOs resulted in the most decreased expression of SEAL1 and various myCAF markers, whether these ASOs affect cell viability was next investigated. As shown in FIG. 5, microscopic images were taken of HDFs after 48 hours of TGFβ induction and SEAL1 ASO transfection or Scrl. Transfection of Scrl, SEAL1_G4, or SEAL1_G5 ASOs resulted in high RNA yield and moderate to low toxicity, respectively, compared to SEAL1_G2 ASO, which showed relatively higher toxicity. Taken together, these data suggested that SEAL1_G4 and SEAL1_G5 ASOs have high efficiency and relatively low toxicity. To confirm such results, qPCR analysis, performed as described in Example 2, measuring expression of SEAL1 and myCAF markers (LRRC15, COL5A2, COL11A1, COL12A1, CTHRC1, MMP11, PDPN, and FAP) was conducted with TGFβ induced CAFs transfected with Scrl, SEAL1 G4, or SEAL_G5. Transfection of SEAL1_G4 and SEAL1_G5 ASOs lead to reduced expression of SEAL1 compared to Scrl (FIG. 6A). In addition, transfection of SEAL1_G4 and SEAL1_G5 ASOs lead to statistically significant reduced expression of LRRC15 (FIG. 6B), COL5A2 (FIG. 6C), COL11A1 (FIG. 6D), CTHRC1 (FIG. 6F), and FAP (FIG. 6I). Transfection of SEAL1_G5 ASO further lead to statistically significant reduced expression of COL12A1 (FIG. 6E), MMP11 (FIG. 6G), and PDPN (FIG. 6H).

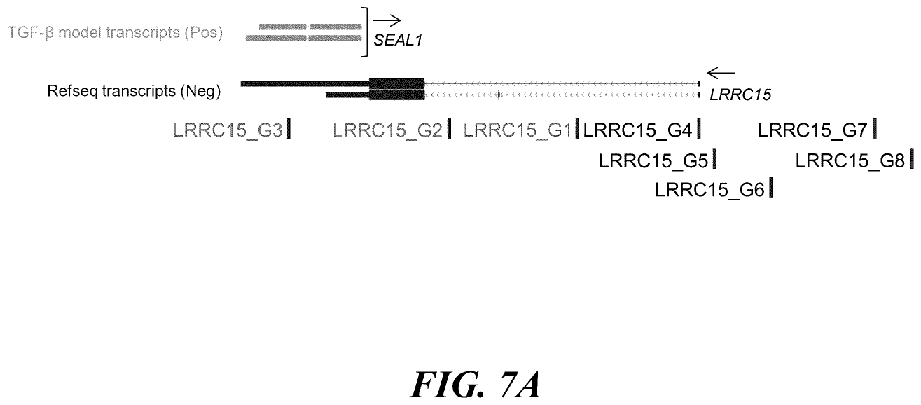

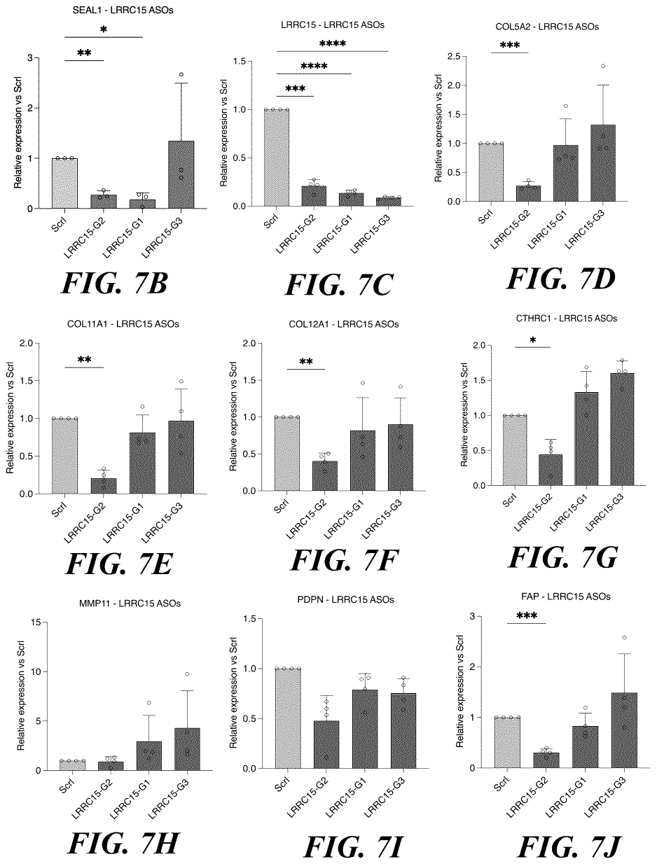

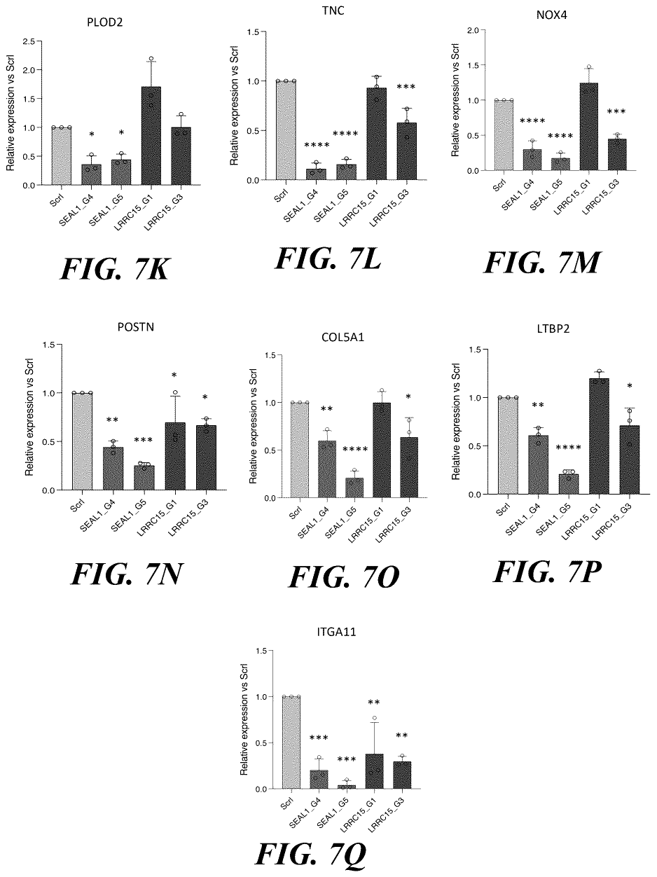

RNA-seq was performed with TGFβ induced CAFs transfected with Scrl, SEAL1_G4, or SEAL1_G5. Similar to FIG. 6A, RNA-sequencing showed that transfection of SEAL1_G4, or SEAL1_G5 reduced expression of SEAL1 compared to Scrl. (FIG. 6J). With the RNA-seq data, gene expression patterns of TGFβ induced CAFs transfected with Scrl, SEAL1_G4, or SEAL1_G5 were analyzed using the signature myCAF gene set with Singscore, wherein a higher score indicates that the pattern of gene expression in a sample is concordant with the pattern in the myCAF signature gene set. As shown in FIG. 6K, TGFβ induced CAFs transfected with SEAL1_G4, or SEAL1_G5 showed decreased Singscore of myCAF signature gene set compared to Scrl, indicating that inhibition or downregulation of SEAL1 by ASO treatment reduced gene expression pattern of the myCAF signature gene set. Gene ontology enrichment analysis was also conducted with genes that were downregulated by both SEAL1_G4 ASO and SEAL1_G5 ASO treatment. Downregulated genes were enriched in myCAF-related functions, such as extracellular matrix structure organization (e.g., extracellular matrix organization, extracellular structure organization, external encapsulating structure organization), and regulation or activation of GTPase activity, suggesting the potential role of SEAL1 in CAF pro-tumorigenic function. TGFβ induced CAFs transfected with SEAL1_G4, SEAL1 G5, LRRC15_G1 (5′CCACTTAGTAATCAGC (SEQ ID NO: 2071)), LRRC15_G3 (5′CTGGGTTAAGATTGGC (SEQ ID NO: 2072)) or Scrl control ASO were evaluated after 48h for the expression of myCAF markers (PLOD2, TNC, NOX4, POSTN, COL5A1, LTBP2, ITGA11) by qPCR, as shown in FIG. 7K-7Q. qPCR analysis showed that transfection of SEAL1_G4 and SEAL1_G5 ASOs led to statistically significant reduced expression of PLOD2 (FIG. 7K), TNC (FIG. 7L), NOX4 (FIG. 7M), POSTN (FIG. 7N), COL5A1 (FIG. 7O), LTBP2 (FIG. 7P) and ITGA11 (FIG. 7Q) markers compared to Scrl. LRRC15_G3 ASO led to statistically significant reduced expression of TNC (FIG. 7L), NOX4 (FIG. 7M), POSTN (FIG. 7N), COL5A1 (FIG. 7O), LTBP2 (FIG. 7P) and ITGA11 (FIG. 7Q) compared to Scrl; however less significant than SEAL1 ASOs. LRRC15_G1 ASO led to statistically significant reduced expression of POSTN (FIG. 7N) and ITGA11 (FIG. 7Q) compared to Scrl. Furthermore, expression of the TEAD2, NFATC4 and RUNX1 TFs predicted target PCGs were also more downregulated upon transfecting TGF-β induced myCAFs with the SEAL1_G4 ASO, as compared to the LRRC15_G3 or non-targeting Scrl ASOs (FIG. 22B). Taken together, these results suggest that SEAL1 might have a stronger impact on myCAF markers than LRRC15.

RNA-seq was performed with TGFβ induced CAFs transfected with Scrl, LRRC15_GI and LRRC15_G3 ASOs, and FIG. 9A shows that transfection with LRRC15_G1 and LRRC15_G3 reduced expression of LRRC15 compared to Scrl. The RNA-seq gene expression patterns of TGFβ induced CAFs transfected with Scrl, SEAL1_G4, SEAL1_G5, LRRC15_GI or LRRC15_G3 were analyzed using the defined myCAF signature (Table 13) with Singscore, where a higher score indicates that the pattern of gene expression in a sample is concordant with the pattern in the myCAF signature. As shown in FIG. 9B, TGFβ induced CAFs transfected with SEAL1_G4 or SEAL1_G5 showed a decreased Singscore of the myCAF defined signature, as compared to Scrl and LRRC15_G1 or LRRC15_G3 ASOs. This suggests that SEAL1 has a stronger impact on the myCAF cell identity than LRRC15. Sub-cellular fractionation of TGFβ induced CAFs demonstrated that SEAL1 transcript was predominantly localized in the nucleus, suggesting a transcriptional regulatory role, while LRRC15 mRNA showed only a partial nuclear localization.

To further compare the effect of SEAL1 and LRRC15 ASOs on the myCAF cell identity, control HDFs transfected with Scrl ASO and TGFβ induced CAFs transfected with either Scrl ASO, SEAL1_G4 ASO or LRRC15_G3 ASOs were analyzed with single nuclei RNA-seq (snRNA-seq), which enables quantification of gene expression on single cell level. For this, around 3-7 million cells were harvested for each condition (HDF-Scrl ASO, TGFβ-Scrl ASO, TGFβ-SEAL1_G4 ASO, TGFβ-LRRC15_G3 ASO). For RNA extraction, 15% of cells/condition were put aside. 3 million cells/condition were used for nuclei isolation; the cell pellet was resuspended in 10 ml of swelling buffer and incubated for 5 min at 4° C. The supernatant was discarded and lysis buffer, including SUPERase RNAse inhibitor (Roche), was added to dissolve the cell pellet during 5 min incubation on ice. 30,000 cells/condition were used for snATAC-seq preparation. Single nuclei preparations were barcoded using the 10× chromium platform (Single cell 3′ Reagent Kit v3, 10× Genomics). Post GEM-RT clean-up was performed on barcoded cDNA. 250 million reads/sample were sequenced on a NovaSeq 6000 (Illumina). snRNA-seq raw data was mapped to the GRCh38/hg38 reference genome assembly and processed using 10× Genomics CellRanger (v7.1.0). The DropletQC method was used to calculate the nuclear fraction for each cell and based on the nuclear fraction and high UMI (unique molecular identifier) count, the top 15,000 nuclei were selected for further analysis. Quality control, clustering and data analysis was performed using the Seurat package, and a CellRanger index generated using the transcriptome assembly built for the TGFβ/COMB in vitro model. Expression of selected transcripts was quantified as log normalized expression values. As shown in FIG. 9C, SEAL1 expression was not detected in control HDFs (condition 1), while weak LRRC15 expression was detected. In the TGFβ induced CAFs (condition 2), SEAL1 expression was detected in a sub-population of the TGFβ induced CAFs (FIG. 9C, top left), whereas LRRC15 expression was detected in the entire population (FIG. 9C, top, right). Transfection with SEAL1_G4 ASO (condition 3) effectively removed the SEAL1 transcript (FIG. 9C, top left), but not all the LRRC15 transcript (FIG. 9C, top right). Transfection with LRRC15_G3 ASO (condition 4) effectively removed LRRC15 transcript. Singscore analysis with the defined myCAF signature confirmed that the expression of these genes was clearly induced in the TGFβ induced CAFs (condition 2) as compared to control HDFs (condition1), and markedly reduced in SEAL1_G4 transfected cells (condition 3), respectively (FIG. 9C, bottom left). The signature was not reduced, however, in LRRC15_G3 transfected cells (condition 4, FIG. 9C, bottom left). The defined myCAF signature was confirmed by pseudobulk quantification to be reduced with the SEAL1_G4 ASO (hSEAL1_G4) and not with the LRRC15_G3 ASO (hLRRC15_G3) (FIG. 9C, bottom right side violin plot). These results support the notion that SEAL1 may have a larger impact on the myCAF cell identity than LRRC15.

To investigate the expression of SEAL1 and LRRC15 during HNSCC cancer progression in a scRNA-seq dataset (Choi et al, Single-cell transcriptome profiling of the stepwise progression of head and neck cancer, Nature Communications 14(1), 2023), the SEAL1 lncRNA and LRRC15 mRNA transcripts were quantified using the MAGIC algorithm (Van Dijk et al, Recovering gene interactions from single-cell data using data diffusion. Cell 174(3), 2018) in the FBs of each sample condition (normal tissue, pre-cancerous leukoplakia and primary cancer). As shown in FIG. 9D, SEAL1 lncRNA was only expressed in primary cancer FBs, corresponding to the FB sub-cluster 3 myCAFs (FIG. 9D, top), while LRRC15 mRNA was expressed in leukoplakia FBs as well as primary cancer FBs (FIG. 9D, bottom). This suggests that SEAL1 may be more specific for the fully activated myCAF cell state (in primary cancer FBs) than LRRC15. An independent HNSCC scRNA-seq dataset (Puram et al, Single-cell transcriptomic analysis of primary and metastatic tumor ecosystems in head and neck cancer. Cell 171(7), 2017) analyzed in Example 1 also validated the expression of SEAL1 and LRRC15 in sub-cluster of fibroblasts that represent fully differentiated myCAF cell state. Furthermore, since triple-negative breast cancer (TNBC) is another solid tumor type with a recognized myCAF component, a TNBC bulk RNA-seq dataset (Jiang et al, Genomic and Transcriptonic Landscape of Triple-Negative Breast Cancers: Subtypes and Treatment Strategies. Cancer Cell 35(3), 2019) was queried for SEAL1 and LRRC15 expression. Both SEAL1 and LRRC15 were expressed in tumor samples (n=119) and not in normal tissue (n=20) samples. To further confirm SEAL1 expression on the single cell level, a single nuclei RNA-seq (snRNA-seq) dataset on 33 patients from the Human Tumor Atlas Network (HTAN) consortium was analyzed. SEAL1 and LRRC15 were detected in a sub-population of fibroblasts that also expressed the in-house defined myCAF signature as well as the SEAL1 module signature. These results showed that SEAL1 may be expressed in a similar myCAF population, without expression in normal tissue or leukoplakia FBs, as was confirmed in HNSCC (FIG. 9D). In contrast, LRRC15 displayed a weak expression some of the normal tissue FBs and clear induction in leukoplakia FBs. LRRC15 was also found to be expressed in primary cancer FBs, however less than in leukoplakia FBs. While SEAL1 and LRRC15 both displayed a CAF-associated expression pattern, SEAL1 appeared to surpass LRRC15 with regards to primary cancer myCAF specificity.

For analysis of SEAL1_G4 and LRRC15_G3 ASO dose-response, TGFβ induced CAFs were transfected as described above with 0, 0.5, 1, 5, 10 and 25 nM SEAL1_G4, LRRC15_G3 ASOs or control Scrl ASOs. RNA was extracted and analyzed by RNA-seq. The SEAL1_G4 and LRRC15_G3 ASOs dose response RNA-seq analysis was done using the R package drc (dose-response curves), giving the IC₅₀ ASO concentration for the target expression (achieving the half maximal inhibitory effect) and the RC₅₀ ASO concentration for the expression of downstream targets (achieving half reduction in expression level). Statistical significance was calculated by t-test between data and model. As shown in FIG. 9E-9G, bulk RNA-seq analysis was performed to investigate the expression of SEAL1, myCAF markers and myCAF signature gene set (Table 13) upon transfection with SEAL1_G4 versus Scrl. RNA-seq analysis showed that SEAL1 expression displayed a statistically significant ASO dose response reduction, with an IC₅₀ ASO concentration determined to be 4.65 nM, indicating a high efficacy of the SEAL1_G4 ASO (FIG. 9E). Transfection of the SEAL1_G4 ASO also caused a statistically significant ASO dose response reduction in the expression of the myCAF markers LRRC15, FAP, CTHRC1, POSTN, LTBP2, with RC₅₀ values determined to be 4.89 nM for LRRC15, 4.83 nM for FAP, 3.42 nM for CTHRC1, 3.98 nM for POSTN, and 4.99 nM for LTBP2 (FIG. 9F and FIG. 9S). Decreasing trend in the expression of TNC and COL5A1 was observed with varying dosage of SEAL_G4 ASO (FIG. 9S). Similarly, Singscore calculation of the myCAF signature set also displayed a statistically significant ASO dose response reduction to the SEAL1_G4 ASO transfection, with an RC₅₀ of 4.99 nM (FIG. 9G). A lower Singscore value of the myCAF signature corresponds to a lower expression, as determined by RNA-seq analysis. As shown in FIGS. 9H-9J, bulk RNA-seq analysis was performed to investigate the expression of LRRC15, myCAF markers and myCAF signature gene set upon transfection with LRRC15_G3 versus Scrl. RNA-seq analysis showed that LRRC15 expression displayed a statistically significant ASO dose response reduction, with an IC₅₀ ASO concentration determined to be 1 nM, indicating a high efficacy of the LRRC15_G3 ASO (FIG. 9H). Transfection of the LRRC15_G3 ASO caused a statistically significant ASO dose response reduction in the expression of the myCAF marker POSTN, with an RC₅₀ value of 2.97 nM (FIG. 9I). The expression of SEAL1 and the other myCAF markers FAP and CTHRC1 was reduced but did not show a dose response reduction to the LRRC15_G3 ASO (FIG. 9I). Similarly, Singscore calculation of the myCAF signature showed a minor reduction, but not a dose response reduction to the LRRC15_G3 ASO (FIG. 9J). Taken together, these results suggest that the myCAF markers, and the myCAF signature, are reduced in direct response to the SEAL1_G4 ASO, but not to the LRRC15_G3 ASO. The RNA-seq analysis of TGFβ induced CAFs transfected as described above with 0, 0.5, 1, 5, 10 and 25 nM SEAL1_G4 ASO was then analyzed to define a gene signature that displays a dose response to the SEAL1_G4 ASO. An RNA-seq dose response cluster analysis was performed, using unbiased hierarchical clustering (k=7), to identify gene clusters showing SEAL1_G4 ASO dose responsive changes in expression (FIG. 9K). 7 gene clusters that displayed gene expression changes upon SEAL1_G4 ASO transfection were identified: clusters 7 (n=90), 1 (n=790 genes), 3 (n=4598) and 5 (n=3180) were upregulated, while clusters 2 (n=3833), 4 (n=1704) and 6 (n=526) were downregulated (FIG. 9K, left). The Cluster 6 genes (n=526) showed the strongest downregulation in response to SEAL1_G4 ASO and was referred to as the SEAL1 Target Engagement panel (SEAL1 TEP, Table 14). Singscore analysis of the SEAL1 TEP demonstrated a significant SEAL1_G4 ASO dose responsive reduction with an RC₅₀ value of 4.4 nM (FIG. 9K, right). Gene ontology enrichment analysis was conducted with the SEAL1 TEP signature genes (n=526, Table 14) that displayed SEAL1_G4 ASO dose responsive reduction. The SEAL1 TEP genes were enriched in myCAF-related functions, such as neuron projection development, axon development, semaphorin-plexin signaling pathway, collagen-containing extracellular matrix, extracellular matrix structural constituent, suggesting the potential role of SEAL1 in CAF pro-tumorigenic functions. In particular, neurodevelopmental and axonogenesis pathways have been described to be CAF-related features involved in innervation of the TME, a process linked to perineural invasion and poor patient prognosis. To investigate the expression of the SEAL1 TEP during HNSCC cancer progression, the SEAL1 TEP was analyzed by Singscore in all FB sub-clusters (FIG. 9L, top left) of the HNSCC human patient samples. As shown in FIG. 9L, the fibroblast sub-cluster with the highest (meaning higher expression) SEAL1 TEP score (FIG. 9L, top right) is the same fibroblast sub-cluster that displays expression of the SEAL1 lncRNA (FIG. 9L, top middle), and corresponds to the fibroblast sub-cluster which constitutes the myCAFs (FIG. 9L, top left). FIG. 9L bottom left and bottom right similarly show a FB-specific hSEAL1 module linked to myCAF pathways that displayed specific expression in primary cancer myCAFs. To validate the association of SEAL1 with pro-tumorigenic functions, a high dimensional weighted gene co-expression network analysis (hWGCNA) was performed using the HNSCC cancer progression scRNA-seq dataset (Choi et al, Single-cell transcriptome profiling of the stepwise progression of head and neck cancer, Nature Communications 14(1), 2023). This analysis identified gene modules that have a similar expression pattern in an unbiased manner, considering the expression of all protein-coding genes present, and the expression of CAF lncRNAs (Table 10) in all cells of the dataset. The analysis identified 41 fibroblast-specific co-expression modules, whereof one of them (module M1) contained SEAL1, LRRC15, SEAL9 and 177 PCGs. The SEAL1 module signature (n=177) was enriched in myCAF-related functions such as extracellular matrix organization, ECM-receptor interaction, collagen formation, synapse organization and TGFβ signaling pathway, similar to the SEAL1 TEP pathway analysis. An unbiased analysis based on human HNSCC patient samples thus confirmed the association of SEAL1 with fibroblast-specific co-expression networks, and specific CAF pro-tumorigenic pathways involving ECM dysregulation and tumor innervation. As shown in FIG. 9M, Singscore analysis showed that the SEAL1 module (n=177) is reduced upon transfecting TGFβ induced CAFs with SEAL1_G4 ASO (FIG. 9M, left), as compared to control Scrl ASO transfection (FIG. 9M, left). Transfection with LRRC15_G3 ASO (FIG. 9M, left) did not reduce the signature. This result suggests that the expression of the SEAL1 module depends on SEAL1, rather than LRRC15. Singscore analysis of the HNSCC disease progression dataset also showed that the SEAL1 module (n=177) was expressed in primary cancer FBs that correspond to the myCAF sub-cluster (FIG. 9M, right).

An independent HNSCC scRNA-seq dataset was used to validate the abilities of the myCAF signature and SEAL1 expression to identify prognostically relevant myCAFs. FB sub-clustering identified three clusters, 0, 1 and 2, whereof sub-cluster 1 expressed SEAL1. Singscore analysis of the myCAF signature and SEAL1 co-expression module confirmed the SEAL1-expressing sub-cluster 1 to represent the fully differentiated myCAF cell state, corresponding to the sub-cluster 3 identified in the HNSCC cancer progression dataset. The cell type and FB sub-cluster information was used to deconvolute the TCGA-HNSC patient cohort, partitioning the patients based on having a high or low sub-cluster 1 myCAF fraction content, respectively (FIG. 21B). The sub-cluster 1 myCAF fraction—high patient category was associated with significantly (p=0.02) worse OS as compared to sub-cluster 1 myCAF—low patient category, similarly to the HNSCC cancer progression dataset sub-cluster 3 myCAF fraction survival analysis (FIG. 21A). This finding validates the association of the myCAF signature, and SEAL1 expression, with myCAFs of prognostic value.

To verify that the effect of ASO-mediated SEAL1 targeting was independent from LRRC15, LRRC15 was depleted by the clustered regularly interspaced short palindromic repeats—inhibition (CRISPRi) technique. To enable a prolonged HDF culturing required for the CRISPRi experiment, primary HDFs cultured, as described in Example 2, and at 50% confluency were transfected with 1 l SV40 viral supernatant (ALSTEM) and 4 l TransPlus reagent (ALSTEM) in antibiotic-free FGM-2 medium to generate an immortalized HDF cell line (iHDF). Medium was changed to complete FGM-2 medium the next day and 48 hours after transfection the cells were selected with 1 g/ml Puromycin (ThermoFisher) during 10 days. After 10 days of Puromycin selection, the medium was replaced with complete FGM-2 medium. Next, dCAS9-iHDF cells were generated by transfecting the iHDF line with lentiviral hCMV-Blast-dCas9-SALL1-SDS3 particles (Horizon Discovery) at an MOI of 0.3 with 4 μL TransPlus reagent. Medium was changed the next day with fresh antibiotic-free FGM-2 medium and 48h after transfection the cells were selected with 4 □g/ml Blasticidin (Fisher Scientific) during 10 days. After 10 days, the medium was replaced with complete FGM-2 medium. Guide RNAs (sgRNAs) targeting the LRRC15 transcription start site were designed using CRISPick design module from the Broad institute, cloned into the CRISPRi lentiviral non-targeting control plasmid (Horizon Discovery) and used to generate viral particles together with packaging plasmids (Trono lab) in HEK293 cells. The following sgRNAs were used for the annotated LRRC15 transcriptional start site (TSS): TSS_G2 (5′TCAAGGCTGCAGCATGAGTG (SEQ ID NO: 2073)), TSS_G4 (5′GGAGCCTGAGAGGAGGACGA (SEQ ID NO: 2074)), TSS_G5 (5′GTGGCTCGCCTAAGCTGTCC (SEQ ID NO: 2075)), TSS_G6 (5′GTACCCTGTAGTGTCAGCCC (SEQ ID NO: 2076)), TSS_G8 (5′GCCGGGCCCTCTAAGCAGAG (SEQ ID NO: 2077)) and TSS_G9 (5′GGTCCAGCAGAAATGGGAAG (SEQ ID NO: 2078)), and for the alterative TSS: alt_TSS_GI (5′GGGCGGGCTCAGCAGTGATG (SEQ ID NO: 2079)) and alt_TSS_G3 (5′AGGTAGAATAGATCCAGCCG (SEQ ID NO: 2080)). To generate lentiviral particles with sgRNA plasmids, HEK cells were transfected with packaging plasmids psPAX2 and pMD2.G, and one unique sgRNA plasmid, per well using the X-tremeGENE HP DNA Transfection Reagent (Roche) in Optimem medium during 4h. 4h after transfection, the medium was replaced with complete FGM-2 medium, and 24h later, the medium was collected as viral supernatant, filtered through a 40 μm strainer and stored at −80° C.. The dCAS9-iHDF cell line was transduced using 4 μL TransPlus reagent in complete FGM-2 medium with viral particles carrying the sgRNAs targeting LRRC15, versus non-targeting sgRNAs. Medium was changed the next day. The cells were then induced by TGFβ and transfected with SEAL1_G4 versus control Scrl ASOs, as described above. Cells were harvested 48h after ASO transfection for RNA-seq and western blot analysis, and RNA was extracted, as described above. As shown in FIGS. 9N-9P, bulk RNA-seq analysis was performed to investigate the effect of ASO-mediated SEAL1-targeting independent from LRRC15. For this, non-TGFβ induced (control) dCAS9-iHDF cells were transfected with non-targeting sgRNA plus non-targeting Scrl ASO. TGFβ-induced dCAS9-iHDF cells were transfected with either non-targeting sgRNA plus Scrl ASO (TGFβ control), with LRRC15-targeting sgRNA plus Scrl ASO (LRRC15 depletion only), with non-targeting sgRNA plus SEAL1_G4 ASO (SEAL1 depletion only) or with LRRC15-targeting sgRNA plus SEAL1_G4 ASO (depletion of both LRRC15 and SEAL1). The LRRC15-targeting sgRNAs effectively reduced LRRC15 expression 83% (FIG. 9N), compared to non-targeting sgRNAs plus Scrl ASO (FIG. 9N). The LRRC15-targeting sgRNAs plus SEAL1_G4 ASO also effectively reduced SEAL1 expression in TGFβ induced cells, compared to non-targeting sgRNAs plus Scrl ASO (FIG. 9R) in TGFβ induced cells. The LRRC15+ myofibroblast marker signature (Buechler et al, Cross-tissue organization of the fibroblast lineage. Nature 593 (7860), 2021) score was also significantly reduced in TGFβ induced cells treated with both LRRC15-targeting sgRNAs plus SEAL1_G4 ASO compared to TGFβ induced cells treated with non-targeting sgRNA plus Scrl ASO. Of note, this signature was not reduced in TGFβ induced cells treated with LRRC15-targeting sgRNAs plus Scrl ASO, meaning that these markers responded to SEAL1 depletion rather than LRRC15 depletion. The myCAF markers COL11A1, CTHRC1, COL5A2 and ITGA11 were effectively reduced only when the cells were also transfected with the SEAL1_G4 ASO (FIG. 9O). In the cells with LRRC15 depletion only, meaning transfection with LRRC15-targeting sgRNA plus Scrl ASO, the markers only showed a minor reduction (FIG. 9O). Moreover, few genes were differentially expressed in cells with LRRC15 depletion only compared to treatment with non-targeting sgRNAs plus Scrl ASO. Only two gene ontology pathways were enriched among the downregulated protein coding genes, of which only one was associated with myCAF function (e.g., collagen fibril organization pathway). Several pathways were identified among the genes upregulated by depletion of LRRC15, but were associated with immune response functions such as response to bacterium and cytokine production. This suggested that LRRC15 may be related to cellular functions that are unrelated to myCAF tumor-supportive mechanisms. Similarly, Singscore of the myCAF marker signature (Table 13), showed that only the cells with depleted SEAL1, meaning transfection with non-targeting sgRNA plus SEAL1_G4 ASO (SEAL1 depletion only) or with LRRC15-targeting sgRNA plus SEAL1_G4 ASO (depletion of both LRRC15 and SEAL1), significantly reduced the myCAF signature score (FIG. 9P). The cells with LRRC15 depletion only did not reduce the myCAF signature. Taken together, these results demonstrated that SEAL1-targeting reduced myCAF marker expression independent from LRRC15 expression level, and that LRRC15-targeting only did not reduce myCAF marker expression.

As shown in FIG. 9Q, western blot analysis to detect the LRRC15 protein was performed. Around 200,000 cells from each condition were collected and lysed in 70 μl RIPA buffer (1×: 50 mM Tris-HCl pH 8, 150 mM NaCl, 1% NP40, 0.5% NaDOC, 0.1% SDS) supplemented with 1× protease inhibitor (Sigma) and incubated 30 minutes on ice. The cell lysates were centrifuged 15 min at 13,000 rpm and 4° C. and the protein content measured in the supernatant using the Pierce BCA kit (ThermoFisher). Protein electrophoresis was performed in NuPAGE™ 12% Bis-Tris gels and MOPS SDS Running Buffer (20×) (ThermoFisher), with 10 μg of protein from each sample. Blotting was performed using the iBlot2 transfer system and nitrocellulose membranes (ThermoFisher). Proteins detection was performed using anti-LRRC15 (ab150376, Abcam) or anti-GAPDH antibody (ab8245, Abcam) primary antibodies, HRP-conjugated secondary antibodies (anti-mouse, ThermoFisher, anti-rabbit, CellSignal) and the SuperSignal™ West Femto Maximum Sensitivity Substrate (ThermoFisher). Proteins were visualized with an Amersham ImageQuant™ 500 CCD imaging system (Cytiva) and quantified relative the control GAPDH with ImageJ. The conditions analyzed were the following: control dCAS9-iHDF cells or TGFβ-induced dCAS9-iHDF cells transfected with non-targeting Scrl ASO, with LRRC15-targeting sgRNA plus Scrl ASO (LRRC15 depletion only), with non-targeting sgRNA plus SEAL1_G4 ASO (SEAL1 depletion only) or with LRRC15-targeting sgRNA plus SEAL1_G4 ASO (depletion of both LRRC15 and SEAL1). The LRRC15 protein was effectively reduced in cells with LRRC15 depletion only (FIG. 9Q). The cells with SEAL1 depletion only (FIG. 9Q) also reduced LRRC15 protein levels; however less than with LRRC15 depletion only or depletion of both LRRC15 and SEAL1. These results demonstrated that SEAL1 reduced the myCAF marker signature (FIG. 9P), independent from LRRC15 protein expression.

Since there are overlapping SEAL1 and LRRC15 regions on opposite strands (e.g., antisense transcription orientation), the effect of LRRC15 knockdown on myCAF expression was investigated to see if SEAL1 or LRRC15 regulate expressions of myCAF markers. Different ASOs against LRRC15 were designed as shown in FIG. 7A. LRRC15_G1-G4 were designed in the exonic and intronic regions of LRRC15, and LRRC15_G5-G8 were designed in the upstream regulatory region. HDFs were induced with TGFβ and then transfected with LRRC15 ASOs, using the same protocol as with SEAL1 ASOs. Transfected cells were harvested for RNA extraction and subsequent qPCR analysis, performed as described in Example 2, was performed with myCAF markers (LRRC15, COL5A2, COL11A1, COL12A1, CTHRC1, MMP11, PDPN, and FAP). As shown in FIG. 7C, expression of LRRC15 was reduced with transfection of all LRRC15 ASOs. However, as shown in FIGS. 7D-7J, only LRRC15_G2 led to statistically significant reduced expression of some myCAF markers (e.g., COL5A2, COL11A1, COL12A1, CTHRC1, and FAP) compared to Scrl. This is different from what was seen with SEAL1 ASOs, suggesting that SEAL1 may regulate myCAF markers more than LRRC15. Interestingly, as shown in FIG. 7B, expression of SEAL1 was increased with LRRC15_G3, implying a possible co-regulatory mechanism, such as by lncRNA-mRNA interaction, lncRNA-protein interaction, or lncRNA-DNA interaction.

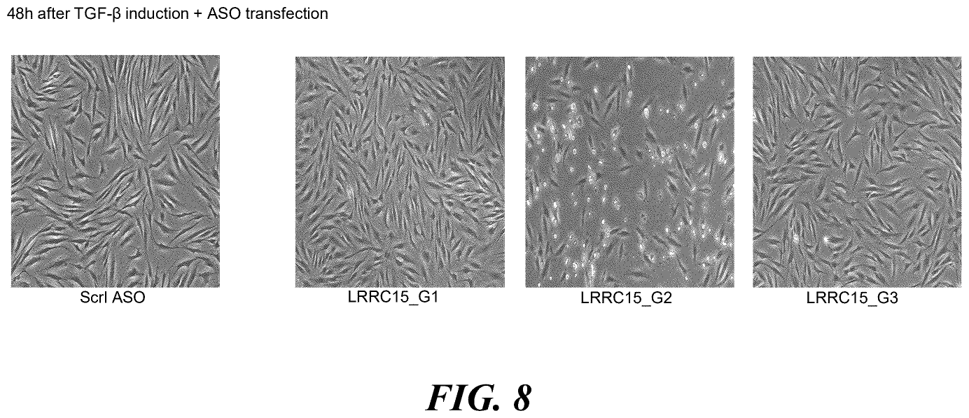

To check whether cell viability was affected by transfection of LRRC15 ASOs, microscopic images of HDFs were taken after 48 hours of TGFβ induction and LRRC15 ASO transfection or Scrl. As shown in FIG. 8, transfection with Scrl, LRRC15_G1, or LRRC15_G3 displayed low toxicity, as compared to LRRC15_G2.

Additional functional genomics methods, such as PRO-Cap analysis to map SEAL1 and LRRC15 transcription initiation upon HDF induction and Cut&Run analysis to map chromatin activation marks (e.g., H3K27ac) upon HDF induction is performed to understand the potential co-regulatory interaction and mechanism between LRRC15 and SEAL1.

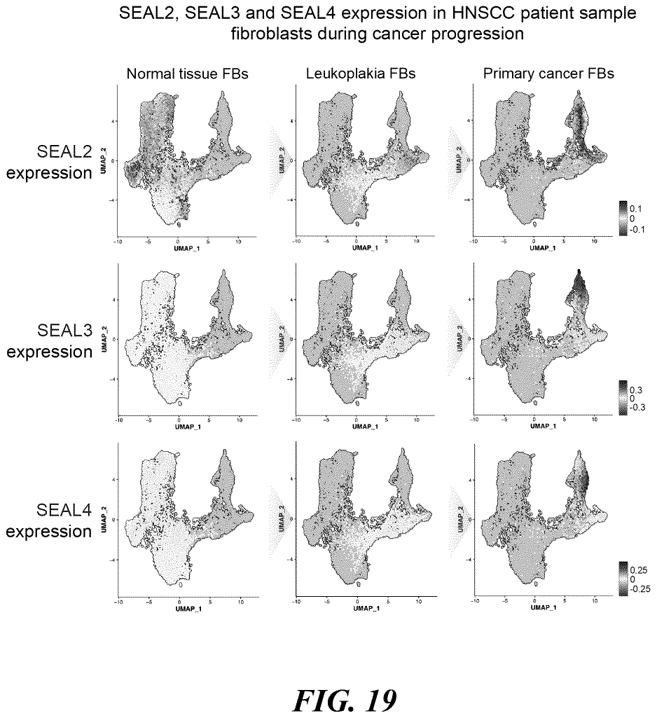

The expression of SEAL2, SEAL3 and SEAL4 was investigated in the human HNSCC cancer progression model described in Example 1 (Choi et al, Single-cell transcriptome profiling of the stepwise progression of head and neck cancer, Nature Communications 14(1), 2023). The SEAL2, SEAL3 and SEAL4 lncRNAs were quantified with MAGIC algorithm (Van Dijk et al, Recovering gene interactions from single-cell data using data diffusion, Cell 174(3), 2018) in the FBs of each sample condition (normal tissue, pre-cancerous leukoplakia and primary cancer). As shown in FIG. 19, the SEAL2 lncRNA showed some expression in normal tissue FBs, but higher expression in primary cancer FBs (FIG. 19, top), in the FB sub-cluster that corresponds to sub-cluster 3 myCAFs. The SEAL3 and SEAL4 lncRNAs are only expressed in primary cancer FBs, in the FB sub-cluster that corresponds to sub-cluster 3 myCAFs (FIG. 19, middle and bottom). This suggests that SEAL3 and SEAL4 are specific for the fully activated myCAF cell state in HNSCC primary cancer. Then, to quantify lncRNA candidates in pancreatic ductal adenocarcinoma (PDAC), scRNA-seq raw data from 24 PDAC patients (Peng et al, Single-cell RNA-seq highlights intra-tumoral heterogeneity and malignant progression in pancreatic ductal adenocarcinoma, Cell Research 29(9), 2019) were obtained and analyzed as in Example 1 to annotate cell types, identify FBs and sub-cluster FBs. FB sub-clustering identified 6 clusters. The FB sub-cluster 1 was annotated to be myCAF-like based on gene set enrichment of the myCAF signature (Table 13) using the Singscore package. The SEAL2, SEAL3 and SEAL4 lncRNAs were quantified with MAGIC algorithm (Van Dijk et al, Recovering gene interactions from single-cell data using data diffusion, Cell 174(3), 2018) in the FBs. The SEAL2 and SEAL3 lncRNAs were only expressed in the cells that correspond to cluster 1 myCAFs. The SEAL4 lncRNA was expressed in the cluster 1 myCAFs, but also in other FBs. This suggests that SEAL2 and SEAL3 are specific for the fully activated myCAF cell state in PDAC primary cancer.

TABLE 14
The SEAL1 Target Engagement Panel (SEAL1 TEP)
Gene name

ABHD2, ABTB2, ACSL1, ACSS2, ADAM12, ADAMTS3, ADAMTS9, ADAMTSL1, ADCK1, AFF3, AGAP1,
ALKBH3, ANKIB1, ANKRD44, ANO1, ANO6, AP3B1, ARHGAP24, ARHGAP26, ARHGAP6, ARL5A, ASCC3,
ASTN2, ATAD2B, ATF2, ATG7, ATP6V1H, BBS4, BBS5, BBS9, BCAS3, BCR, BMERB1, BMPR1B, BNC2,
BNIP3L, BTBD11, BZW1, BZW1P2, C12orf56, C18orf54, CACNA1C, CACNA2D1, CADPS2, CAP2, CARMIL1,
CCBE1, CCDC3, CCDC91, CCN4, CCNT2-AS1, CDH11, CDH4, CDK5RAP2, CDKAL1, CENPBD1P1, CENPP,
CEP112, CERS6, CH25H, CHCHD3, CHCHD6, CHST11, CHST6, CHSY3, CLOCK, CLSPN, CMSS1, CNKSR2,
CNTN1, COA1, COL11A1, COL15A1, COL24A1, COL7A1, COL8A1, COLEC12, CRYBG1, CSMD2, CTSC,
CUEDC1, CWC27, DDAH1, DDX10, DELEC1, DENND1A, DENND1B, DEPDC1, DIAPH2, DIAPH3, DNAH1,
DNAJC1, DNHD1, DOCK4, DOK6, DPP4, DPP9, DPP9-AS1, DPYD, DPYSL3, DTWD2, DYNC2H1, ECE1,
EEFSEC, EFHD1, EHBP1, EIF4EBP3, ELP3, EML1, ENO1P4, ENOX1, ENPP1, ENSG00000174977,
ENSG00000187951, ENSG00000213204, ENSG00000224407, ENSG00000226833, ENSG00000227992,
ENSG00000228470, ENSG00000230928, ENSG00000232176, ENSG00000242474, ENSG00000251569,
ENSG00000253106, ENSG00000254192, ENSG00000258864, ENSG00000258881, ENSG00000258892,
ENSG00000260482, ENSG00000260578, ENSG00000260917, ENSG00000261130, ENSG00000261448,
ENSG00000261512, ENSG00000263276, ENSG00000267127, ENSG00000267904, ENSG00000268790,
ENSG00000269044, ENSG00000273373, ENSG00000276012, ENSG00000278893, ENSG00000279415,
ENSG00000280202, ENSG00000280206, ENSG00000282057, ENSG00000282936, ENSG00000283228,
ENSG00000283563, ENSG00000284906, ENSG00000284946, ENSG00000285547, ENSG00000285577,
ENSG00000286104, ENSG00000288646, EPG5, EPHB1, EPS15, EVI5, EXOC4, EXOC6B, F2RL1, FAF1, FAM126A,
FAM168A, FAM172A, FAM184B, FAM20A, FAM20C, FAM24B, FANCB, FANCC, FANCE, FAT3, FBN1, FBN2,
FBXL17, FBXL7, FBXO9, FER, FGF14, FGGY, FMNL2, FNDC3B, FOXO1, FOXS1, FRMD5, FSBP, FTO, GAB1,
GALK2, GALNT10, GALNT13, GAS7, GLIS1, GLYCTK, GMDS, GMDS-DT, GNG12, GOLGA4, GPATCH8, GPC6,
GPHN, GRIN2D, GSR, GSTM3, HAUS7, HBS1L, HDAC8, HERC4, HES1, HLCS, HMCN1, HMMR, HYDIN2,
IGF1R, IGF2BP2, IL15, IL1R1, IMMP2L, INHBE, INSYN2B, INTS7, INVS, ISLR2, ITGA10, ITGB3BP, ITGBL1,
JAZF1, JPX, KATNAL1, KATNB1, KCNJ6, KCNK9, KCNMB3, KIAA0753, KIAA1549L, KIF16B, KIF26B, KIFAP3,
KIRREL3, KIZ, KLF12, KLHL29, KMT2C, KMT5B, LAMA4, LARGE1, LARS2, LDB2, LDLRAD3, LDLRAD4,
LIN9, LINC00607, LINC00894, LINC01655, LINC02257, LIPG, LPP, LRBA, LTBP1, LYPLA1, MAGI1, MAMDC2,
MAML2, MAML3, MAP4, MAP7, MAPKAP1, MARCHF7, MAST2, MATN2, MBIP, MBOAT1, MCM4, MDFI,
MDFIC, ME3, MED23, MEIS2, METTL15, MGMT, MIATNB, MICU1, MINAR1, MIR711, MITF, MLLT3, MNAT1,
MRPL48, MSC-AS1, MSRA, MTX2, N4BP2, N4BP2L2, NAALADL2, NALF1, NAXD, NBAS, NCAM1, NCAM2,
NCOA3, NCOA7, NCOR1, NCS1, NDE1, NEGR1, NES, NETO1, NFATC3, NFIB, NGF-AS1, NHS, NHSL2, NINL,
NOL10, NSF, NSFP1, NSL1, NTM, NTN1, NTNG1, NTSR1, NUFIP2, OGFOD2, OMD, OPCML, OSMR, PACS1,
PAICS, PAIP1, PALS2, PAPPA2, PARD3B, PARP4, PCBP3, PDE1C, PDE3B, PDIA4, PGBD5, PHKA2, PICALM,
PKNOX2, PLCB1, PLCL1, PLS3, PLXNA1, PLXNA4, PLXNC1, PNMA2, POLA1, POLN, PORCN, PPIP5K2,
PPM1L, PPP1R12B, PPP1R21, PPP4R3B, PPP6R3, PRC1, PRC1-AS1, PRDM6, PRKCA, PRKG1, PRRC2C, PSD3,
PSMD1, PTPN14, PTPRD, PTPRG, PTPRM, R3HDM1, RAB43, RAB43P1, RABGAP1L, RAD18, RAD51B,
RALGPS1, RAMP1, RBFA, RBM33, RBMS3, RCAN2, RFX3, RGS17, RGS5, RGS7, RIC1, RNF216, RNLS, ROS1,
RPL24P8, RPS6KA3, RSU1, RTKN2, SCARF2, SCART1, SCD5, SCFD2, SCN9A, SEL1L3, SEMA3A, SEMA3C,
SEMA5A, SENP8, SGCD, SGCG, SHROOM4, SHTN1, SIL1, SIPA1L1, SLC10A7, SLC11A2, SLC13A3, SLC16A7,
SLC1A2, SLC1A3, SLC2A13, SLC35A1, SLC35E3, SLC35F6, SLC38A4, SLC39A11, SLC39A8, SLC45A4,
SLC46A3, SLC7A11, SLC9A9, SLCO3A1, SLIT3, SLIT3-AS2, SLK, SMARCA2, SMIM7, SNTB1, SORBS1,
SORCS2, SPATA21, SPC25, SPOCK1, SPON1, SRP19, SRPX, SSBP2, SSC5D, ST6GAL2, STK3, STRN, STX8,
STXBP5, SUCLA2, SULF2, SUPT3H, SVEP1, SYNDIG1, SYNJ2, TATDN1, TBC1D1, TBC1D3C, TBC1D3D,
TBC1D3L, TBC1D5, TBC1D8, TBC1D9, TBCK, TCP11L2, TENM2, TENM3, TENM4, TET2, TEX261, THAP4,
THSD4, TMCO4, TMED5, TMEM117, TMEM168, TMEM198B, TMTC2, TMX2, TNC, TNS1, TPK1, TRAF3,
TREX2, TRHDE, TRIL, TRIM6, TRIM66, TSHZ2, TSHZ3, TSPAN9, TTC28, TTF2, TUB, TULP4, UBAC2, UBE2E1,
UBE2E3, UMAD1, USP25, UTRN, VCAN, VCAN-AS1, VEPH1, VPS13B, WARS2, WDPCP, WDR25, WDR59,
WDR70, WIF1, WNT7B, XYLT1, YKT6, ZCCHC4, ZHX2, ZMYND11, ZNF277, ZNF407, ZNF654, ZNF704,
ZNF714, ZRANB3, ZRSR2P1, ZSCAN5A

### Example 4: Characterization of SEAL2 in COMB Induced CAFs

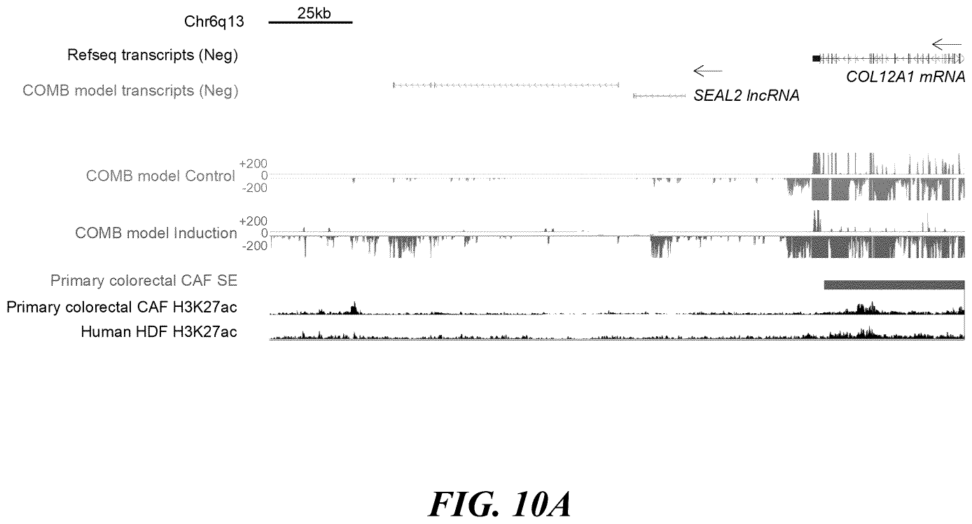

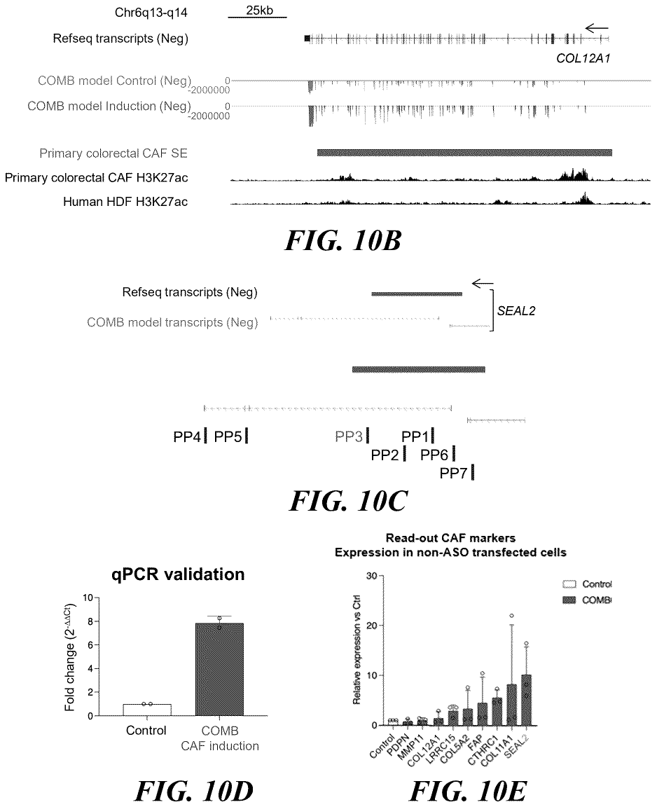

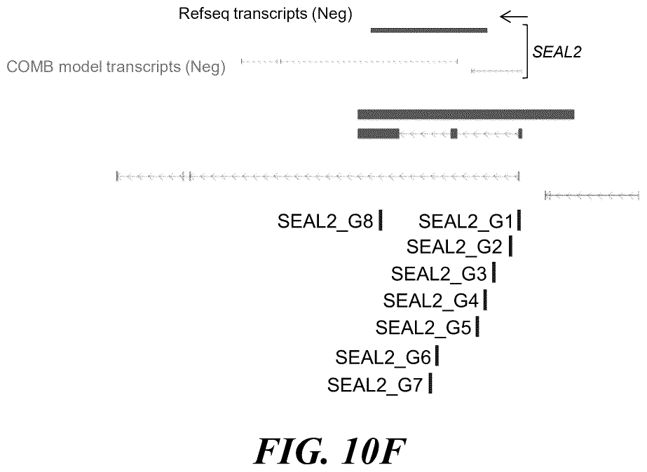

Using the procedure as described in Example 3, genomic locus transcribing SEAL2 was determined. As shown in FIG. 10A, SEAL2 is located in chr6:74958809-75026433 on the anti-sense strand, near a CAF-related protein coding gene, COL12A1. Expression of SEAL2 was upregulated in COMB induced CAFs compared to control. Furthermore, a primary CAF super-enhancer (SE) region was identified upstream of SEAL2, as assessed by the Rank Ordering of Super-Enhancers (ROSE) algorithm and visualized on the genome browser. In addition, since H3K27ac is associated with gene activation, human colorectal cancer CAF and HDF H3K27ac ChIP-seq data were obtained and aligned to GRCh38 genome using Bowtie2 program to observe H3K27ac marks in the SEAL2 region. As shown in FIG. 10A, SEAL2 was associated with H3K27ac signals. FIG. 10B shows a genome region zoomed into the COL12A1 transcript, showing that expression of COL12A1 is upregulated in COMB induced CAFs compared to control, as indicated by greater expression signals. COL12A1 also overlapped with the SE region, suggesting that SEAL2 and COL12A1 may be co-regulated through the SE region. Additional in vitro experiments such as H3K27ac Cut&Run analysis will be performed to identify the presence of an active SE region in the in vitro model.

To test the effect of SEAL2 in COMB induced CAFs, SEAL2 specific primer pairs (PP1-7) were first designed, as shown in FIG. 10C. qPCR amplification was conducted with SEAL2-specific primer pairs and all primer pairs could amplify the SEAL2 transcript, whereof PP3 displayed the best efficiency. Thus, qPCR was performed with PP3 with COMB induced CAFs, which validated SEAL2 expression in COMB induced CAFs compared to control, as shown in FIG. 10D.

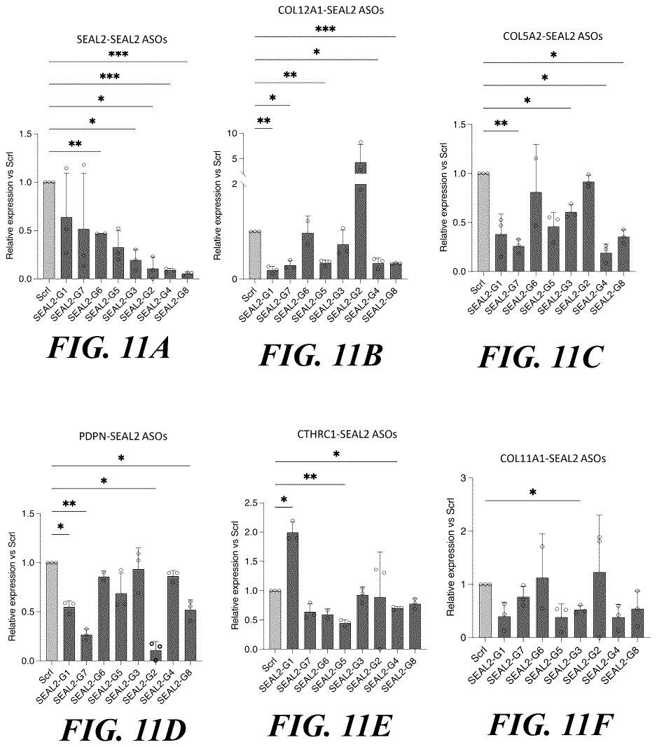

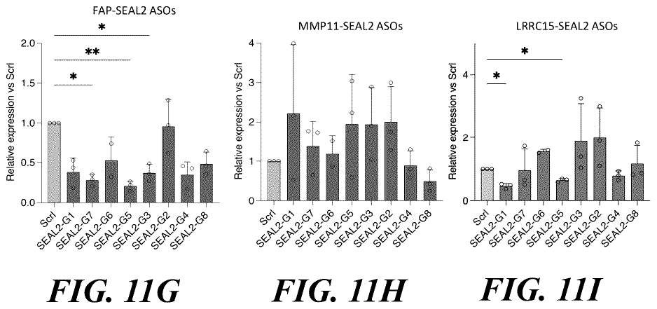

SEAL2 antisense oligonucleotides (ASOs) were also designed as shown in FIG. 10F, to modulate SEAL2 expression in COMB induced CAFs and to investigate the effect of SEAL2 knockdown. Prior to transfecting the cells with the ASOs, read out of myCAF markers and SEAL2 expression were observed in control and COMB induced CAFs via qPCR to establish baseline expression of these markers without ASO treatment, as shown in FIG. 10E. Next, primary HDFs were induced by COMB at the same time as transfection with ASOs. Briefly, 100,000 primary HDFs per well of 6-well plate were plated one day before transfection with SEAL2 ASOs or Scramble control ASO (Scrl). Right before ASO transfection, the cell medium was changed to serum-free medium containing 5 ng/ml TGFβ. ASOs were diluted in TE buffer to a working solution of 20 M and was mixed with OptiMEM medium and X-tremeGENE HP transfection reagent. The mixture was incubated at room temperature for minimum of 15 minutes, and then added dropwise to the cells at 100 l/well, with the total amount of 50 pmol ASO/well. Cells were harvested after 48 hours for RNA extraction and qPCR analysis, as described in Example 2. As shown in FIGS. 11A-11I, qPCR was performed to investigate expression of SEAL2 and myCAF markers (COL12A1, COL5A2, PDPN, CTHRC1, and COL11A1) upon transfection of SEAL2 ASOs versus Scrl. qPCR analysis showed that transfection of most SEAL2 ASOs, resulted in reduced expression of SEAL2 compared to Scrl (FIG. 11A). Transfection of SEAL2_G4, SEAL2_G5, SEAL2_G7, and SEAL2_G8 ASOs lead to decreased expression of several myCAF markers. For example, transfection of SEAL2_G4 ASO resulted in statistically significant reduced expression of COL12A1 (FIG. 11B), COL5A2 (FIG. 11C), and CTHRC1 (FIG. 11E). Transfection of SEAL2_G5 ASO resulted in statistically significant reduced expression of COL12A1 (FIG. 11B), CTHRC1 (FIG. 11E), FAP (FIG. 11G), and LRRC15 (FIG. 11I). Similarly, transfection of SEAL2_G7 or SEAL2_G8 ASO resulted in statistically significant reduced expression of COL12A1 (FIG. 11B), COL5A2 (FIG. 11C), and PDPN (FIG. 11D). Transfection of SEAL2_G7 ASO also resulted in statistically significant reduced expression of FAP (FIG. 11G).

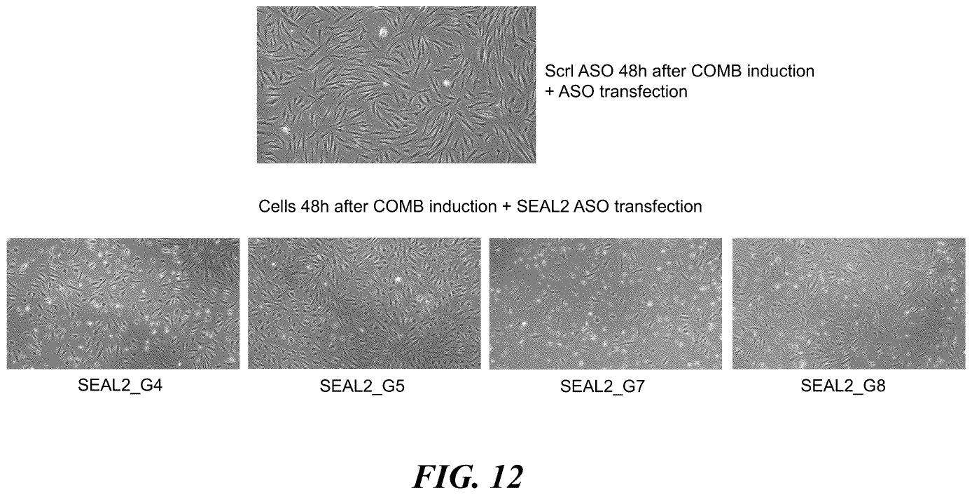

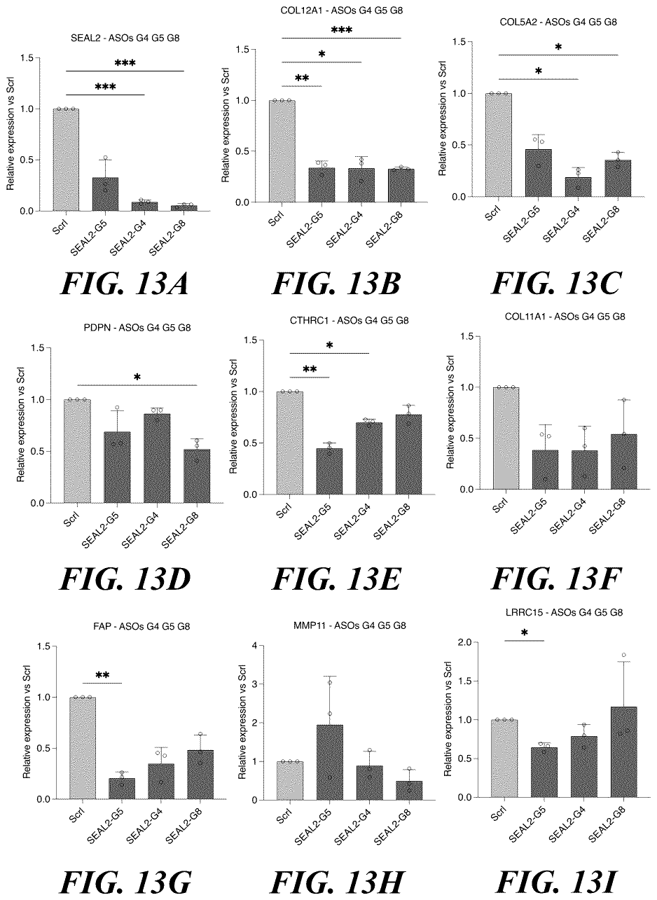

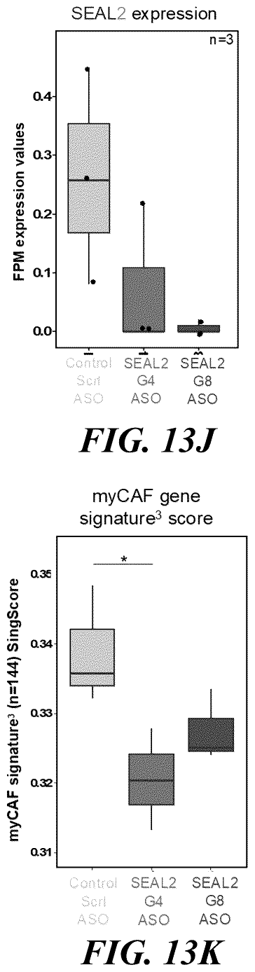

As transfection of SEAL2_G4, SEAL2_G5, SEAL2_G7, and SEAL2_G8 ASOs resulted in the most decreased expression of SEAL2 and various myCAF markers, whether these ASOs affect cell viability was next investigated. As shown in FIG. 12, microscopic images were taken of HDFs after 48 hours of COMB induction and SEAL2 ASO transfection or Scrl. Transfection of Scrl, SEAL2_G4, SEAL2_G5, or SEAL2_G8 ASOs resulted in high RNA yield and low toxicity, compared to SEAL2_G7 ASO. Collectively, these data suggested that SEAL2_G4, SEAL2_G5, and SEAL2_G8 ASOs have high efficiency. To confirm such results, qPCR analysis of expression of SEAL2 and myCAF markers (COL12A1, COL5A2, PDPN, CTHRC1, COL11A1, FAP, MMP11, and LRRC15) was conducted with COMB induced CAFs transfected with Scrl, SEAL2_G4, SEAL2_G5, or SEAL2_G8 ASOs. Transfection of SEAL2_G4, SEAL2_G5, or SEAL2_G8 ASOs lead to reduced expression of SEAL2 compared to Scrl (FIG. 13A). In addition, transfection of SEAL2_G4, SEAL2_G5, or SEAL2_G8 lead to statistically significant reduced expression of COL12A1 (FIG. 13B). Transfection of SEAL2_G4 further lead to statistically significant reduced expression of COL5A2 (FIG. 13C) and CTHRC1 (FIG. 13E). Transfection of SEAL2_G5 ASO further lead to statistically significant reduced expression of CTHRC1 (FIG. 13E), FAP (FIG. 13G), and LRRC15 (FIG. 13I), while transfection of SEAL2_G8 ASO further lead to statistically significant reduced expression of COL5A2 (FIG. 13C) and PDPN (FIG. 13D). These data showed that knockdown of SEAL2 affects expression of myCAF markers, suggesting the potential involvement of SEAL2 in CAFs and CAF pro-tumorigenic function.

RNA-seq was performed with COMB induced CAFs transfected with Scrl, SEAL2_G4, or SEAL2_G8. Similar to FIG. 13A, RNA-sequencing showed that transfection of SEAL2_G4, or SEAL2_G8 reduced expression of SEAL2 compared to Scrl. (FIG. 13J). With the RNA-seq data, gene expression patterns of COMB induced CAFs transfected with Scrl, SEAL2_G4, or SEAL2_G8 were analyzed using the signature myCAF gene set with Singscore, wherein a higher score indicates that the pattern of gene expression in a sample is concordant with the pattern in the myCAF signature gene set. As shown in FIG. 13K, COMB induced CAFs transfected with SEAL2_G4, or SEAL2_G8 showed decreased Singscore of the myCAF signature gene set compared to Scrl, indicating that inhibition or downregulation of SEAL2 by ASO treatment reduced gene expression pattern of the myCAF signature gene set. Gene ontology enrichment analysis was also conducted with genes that were downregulated upon SEAL2_G4 ASO and SEAL2_G8 ASO treatment. Downregulated genes were enriched in myCAF-related functions, such as extracellular matrix structure organization, suggesting the potential role of SEAL2 in CAF pro-tumorigenic function.

### Example 5: Characterization of SEAL3 in TGFβ and COMB Induced CAFs

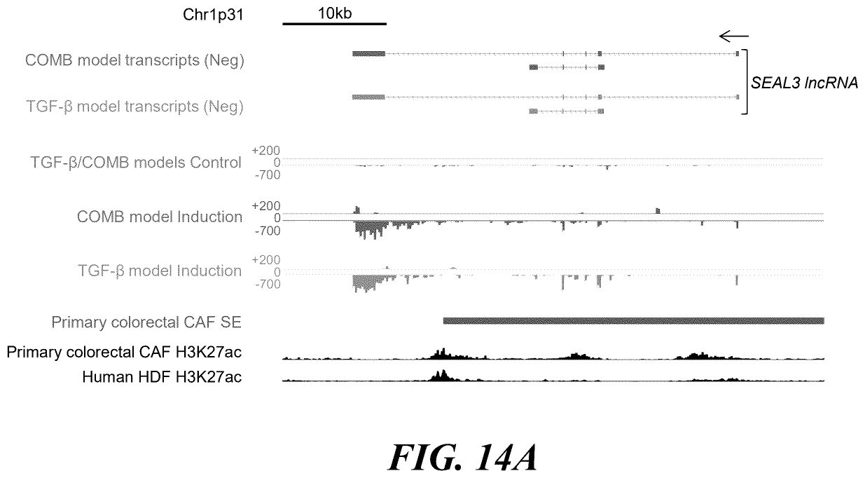

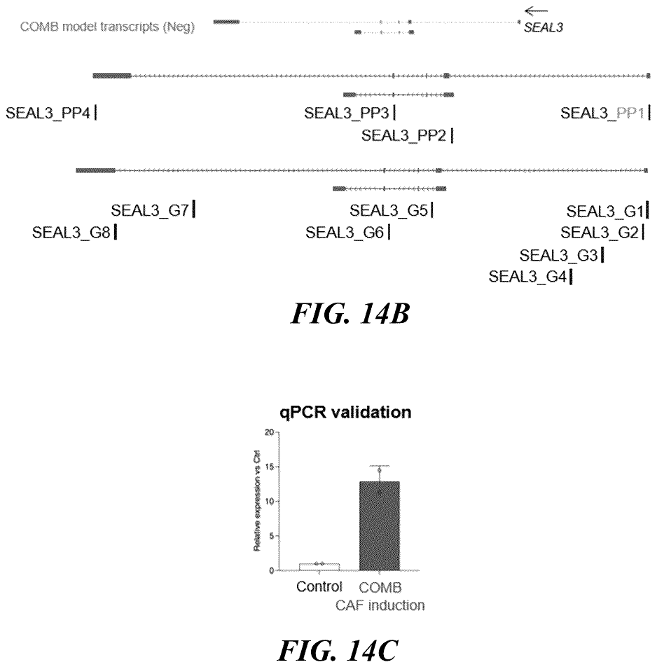

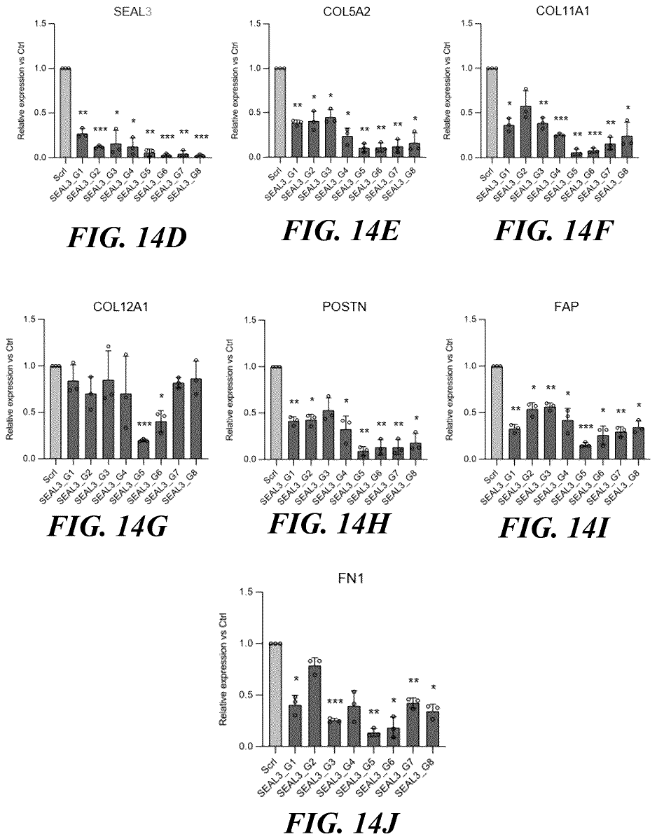

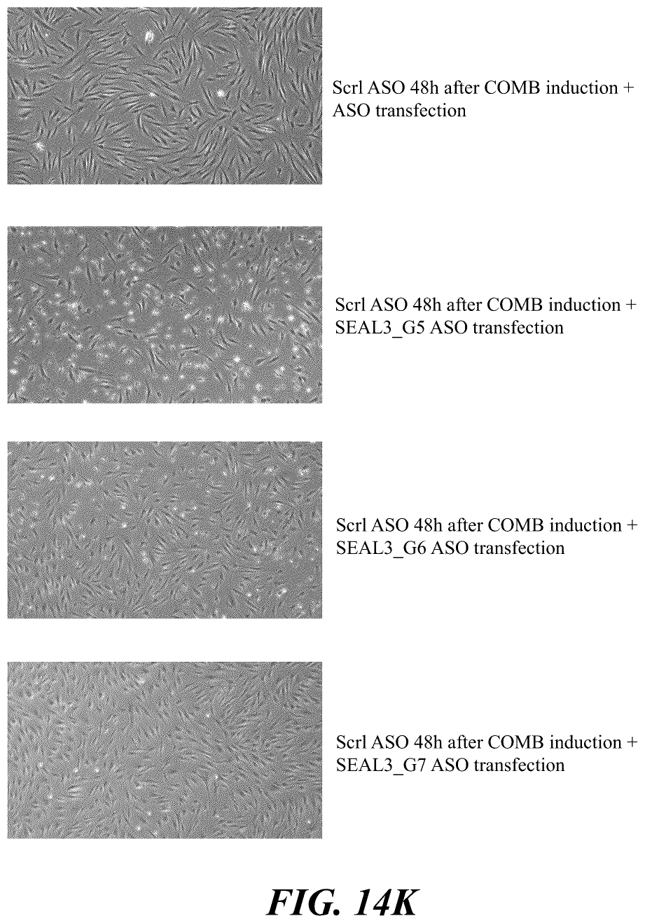

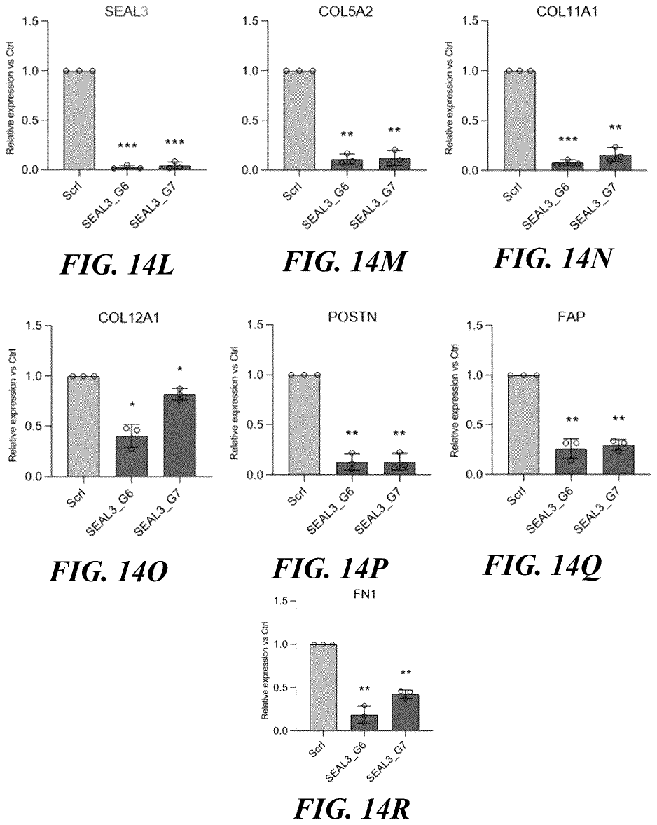

Using the same procedure as described in Example 3, genomic SEAL3 locus was determined. As shown in FIG. 14A, SEAL3 is located in chr1:66390975-66516344 on the anti-sense strand. Expression of SEAL3 was also upregulated in TGFβ/COMB induced CAFs compared to control. No known CAF-related protein coding gene was near SEAL3; however, a primary CAF super-enhancer (SE) region overlapped with SEAL3, as assessed by the ROSE algorithm and visualized on the genome browser. This may indicate a possible CAF-related regulatory control of SEAL3. In addition, since H3K27ac is associated with gene activation, human colorectal cancer CAF and HDF H3K27ac ChIP-seq data were obtained and aligned to GRCh38 genome using Bowtie2 program to observe H3K27ac marks in the SEAL3 region. As shown in FIG. 14A, SEAL3 was associated with H3K27ac signals.

To test the effect of SEAL3 in COMB induced CAFs, SEAL3 specific primer pairs (PP1-4) were first designed, as shown in FIG. 14B, top. Using cDNA synthesized using Quantitect reverse transcription kit, qPCR amplification was conducted with the SEAL3 specific primer pairs. All primer pairs could amplify SEAL3, and PP1 and PP2 displayed the best efficiency. Thus, qPCR was performed with PP1 with COMB induced CAFs, which validated SEAL3 expression in COMB induced CAFs compared to control, as shown in FIG. 14C. SEAL3 specific ASOs were also designed as shown in FIG. 14B, bottom, to modulate SEAL3 expression in COMB induced CAFs. As described in Example 3 and Example 4, the effect of SEAL3 knockdown on TGFβ/COMB induced CAFs can be investigated by using SEAL3 ASOs.

Control HDFs and COMB induced CAFs were transfected with Scrl (control) or SEAL3-targeting ASOs as described in Example 3 and Example 4 and evaluated after 48h for the expression of SEAL3 and myCAF markers (COL5A2, COL11A1, COL12A1, POSTN, FAP, and FN1) by qPCR, as shown in FIGS. 14D-14J. qPCR analysis, performed as described in Example 2, showed that transfection of SEAL3_G1-G8 ASOs led to statistically significant reduced expression of SEAL3 (FIG. 14D) and decreased expression of several myCAF markers compared to Scrl. For example, transfection of SEAL3_G1 resulted in statistically significant reduced expression of COL5A2 (FIG. 14E), COL11A1 (FIG. 14F), POSTN (FIG. 14H), FAP (FIG. 14I) and FN1 (FIG. 14J). Transfection of SEAL3_G2 resulted in statistically significant reduced expression of COL5A2 (FIG. 14E), POSTN (FIG. 14H) and FAP (FIG. 14I), transfection of SEAL3_G3 of COL5A2 (FIG. 14E), COL11A1 (FIG. 14F), FAP (FIG. 14I), and FN1 (FIG. 14J), and transfection of SEAL3_G4 of COL5A2 (FIG. 14E), COL11A1 (FIG. 14F), POSTN (FIG. 14H) and FAP (FIG. 14I). Transfection of SEAL3_G5 and SEAL3_G6 resulted in statistically significant reduced expression of COL5A2 (FIG. 14E), COL11A1 (FIG. 14F), COL12A1 (FIG. 14G), POSTN (FIG. 14H), FAP (FIG. 14I) and FN1 (FIG. 14I). Transfection of SEAL3_G7 and SEAL3_G8 resulted in statistically significant reduced expression of COL5A2 (FIG. 14E), COL11A1 (FIG. 14F), POSTN (FIG. 14H), FAP (FIG. 14I) and FN1 (FIG. 14I). As transfection of SEAL3_G5, SEAL2_G6 and SEAL3_G7 ASOs resulted in the most decreased expression of SEAL3 and various myCAF markers, whether these ASOs affect cell viability was next investigated. As shown in FIG. 14K, microscopic images were taken of HDFs after 48 hours of COMB induction and SEAL3 ASO transfection or Scrl. Transfection of Scrl, SEAL3_G6 or SEAL3_G7 ASOs resulted in high RNA yield and low toxicity, compared to SEAL3_G5 ASO that showed moderate toxicity. Collectively, these data suggested that SEAL3_G6 and SEAL3_G7 ASOs have high efficiency and low toxicity. To confirm such results, qPCR analysis for SEAL3 expression and the expression of myCAF markers (COL5A2, COL11A1, COL12A1, POSTN, FAP and FN1) was conducted with COMB induced CAFs transfected with Scrl, SEAL3_G6 or SEAL3_G7. Transfection of SEAL3_G6 or SEAL3_G7 ASOs lead to reduced expression of SEAL3 compared to Scrl (FIG. 14L). In addition, transfection of SEAL3_G6 or SEAL2_G7 lead to statistically significant reduced expression of COL5A2 (FIG. 14M), COL11A1 (FIG. 14N), COL12A1 (FIG. 14O), POSTN (FIG. 14P), FAP (FIG. 14Q) and FN1 (FIG. 14R). These data showed that knockdown of SEAL3 affects expression of myCAF markers, suggesting the potential involvement of SEAL3 in CAFs and CAF pro-tumorigenic function.

### Example 6: Characterization of SEAL4 in TGFβ and COMB Induced CAFs

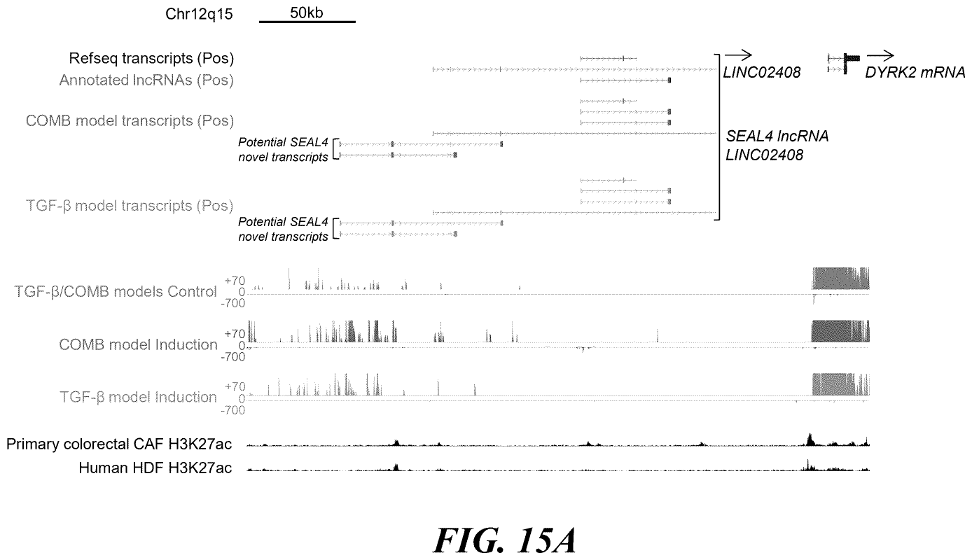

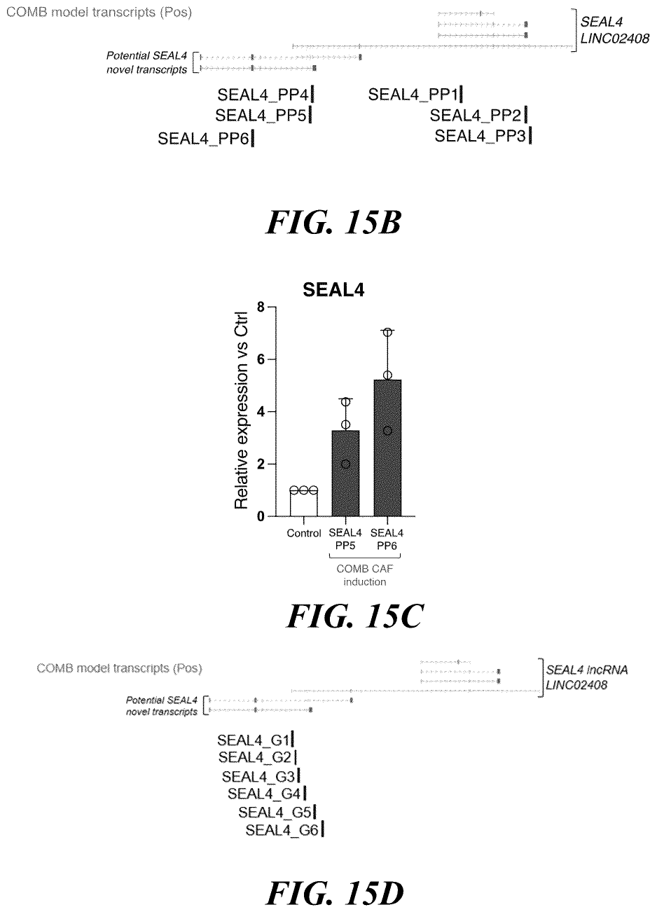

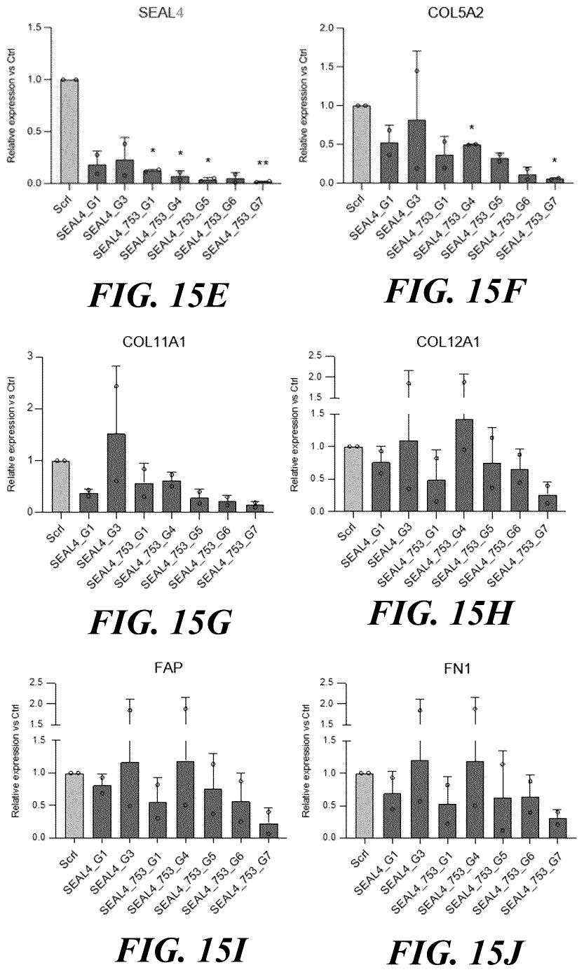

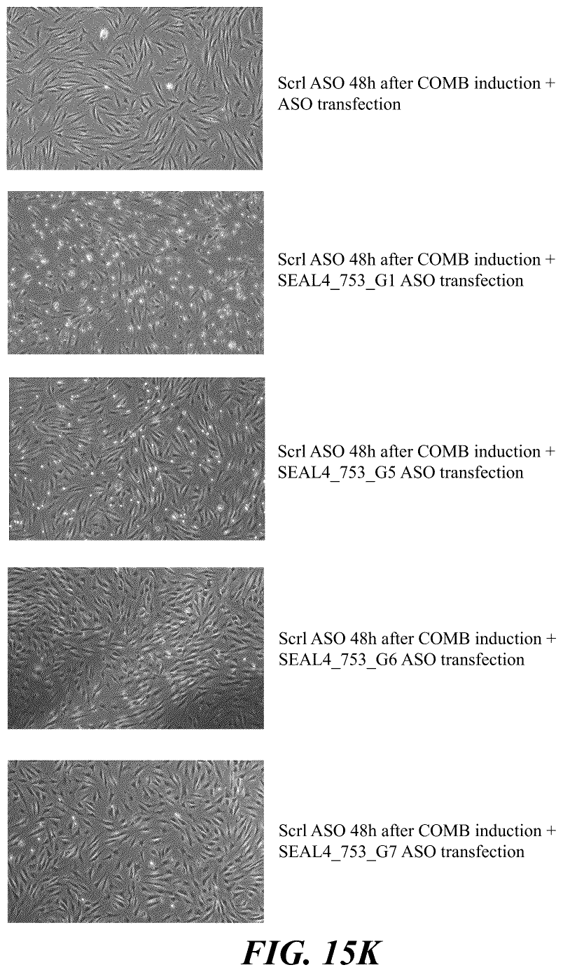

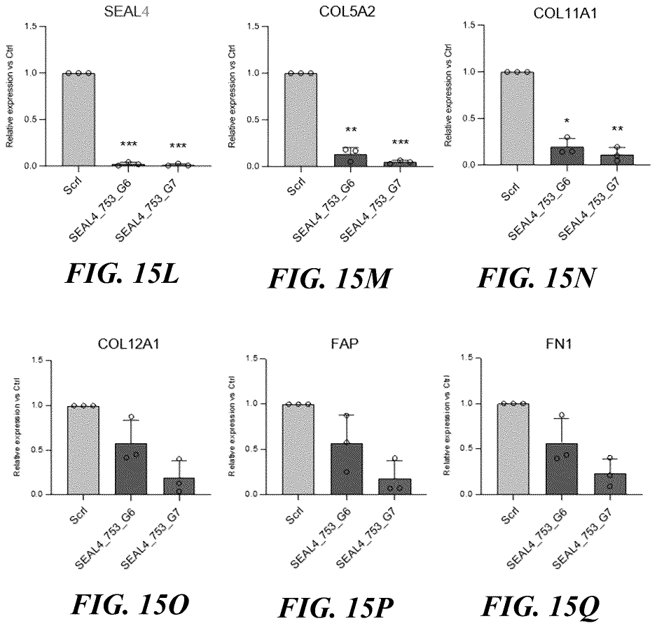

Using the same procedure as described in Example 3, genomic SEAL4 locus was determined. As shown in FIG. 15A, SEAL4 is located in chr12:67394371-67590771 on the sense strand, near the DYRK2 gene. Expression of SEAL4 was also upregulated in TGFβ/COMB induced CAFs compared to control. To be able to test the effect of SEAL4 in COMB induced CAFs, SEAL4 specific primer pairs (PP1-6) were first designed, as shown in FIG. 15B. Using cDNA synthesized using Quantitect reverse transcription kit, qPCR amplification was conducted with the SEAL4 specific primer pairs. All primer pairs could amplify SEAL4, and PP5 and PP6 displayed the best efficiency as shown in FIG. 15C. SEAL4 ASOs were also designed as shown in FIG. 15D, to modulate SEAL4 expression in COMB induced CAFs. As described in Example 3 and Example 4, the effect of SEAL3 knockdown on TGFβ/COMB induced CAFs can be investigated by using SEAL4 ASOs.

Control HDFs and COMB induced CAFs were transfected with Scrl (control) or SEAL4-targeting ASOs as described in Examples 3, 4 and 5 and evaluated after 48h for the expression of SEAL4 and myCAF markers (COL5A2, COL11A1, COL12A1, FAP, and FN1) by qPCR, as shown in FIGS. 15E-15J. qPCR analysis, performed as described in Example 2, showed that transfection of SEAL4 G1, SEAL4 G3, SEAL4_753 G1, SEAL4_753_G4, SEAL4_753_G5, SEAL4_753_G6 and SEAL4_753_G7 ASOs led to reduced expression of SEAL4 (FIG. 15E). SEAL4 ASOs also led to decreased expression of several myCAF markers compared to Scrl. For example, transfection of SEAL4_753_G4 SEAL4_753_G7 resulted in statistically significant reduced expression of COL5A2 (FIG. 15F). Transfection of SEAL4_G1, SEAL4_753_G1, SEAL4_753_G4, SEAL4_753_G5, SEAL4_753_G6 or SEAL4_753_G7 led to decreased expression of COL5A2 (FIG. 15F) and COL11A1 (FIG. 15G). Transfection of SEAL4_G1, SEAL4_753_G1, SEAL4_753_G5, SEAL4_753_G6 or SEAL4_753_G7 led to decreased expression of COL5A2 (FIG. 15F), COL11A1 (FIG. 15G), COL12A1 (FIG. 15H), FAP (FIG. 15I) and FN1 (FIG. 15J). As transfection of SEAL4_753_G1, SEAL4_753_G5, SEAL4_753_G6 and SEAL4_753_G7 ASOs resulted in the most decreased expression of SEAL4 and various myCAF markers, whether these ASOs affect cell viability was next investigated. As shown in FIG. 15K, microscopic images were taken of HDFs after 48 hours of COMB induction and SEAL4 ASO transfection or Scrl. Transfection of Scrl, SEAL4_753_G5, SEAL4_753_G6 and SEAL4_753_G7 ASOs resulted in high RNA yield and low toxicity, compared to SEAL4_753_G1 ASO that showed moderate toxicity. Collectively, these data suggested that SEAL4_753_G5, SEAL4_753_G6 and SEAL4_753_G7 ASOs have high efficiency and low toxicity. To confirm such results, qPCR analysis of SEAL4 expression and myCAF markers (COL5A2, COL11A1, COL12A1, FAP and FN1) was conducted with COMB induced CAFs transfected with Scrl, SEAL4_753_G6 or SEAL4_753_G7 ASOs. Transfection of SEAL4_753_G6 or SEAL4_753_G7 ASOs lead to statistically significant reduced expression of SEAL4 compared to Scrl (FIG. 15L). In addition, transfection SEAL4_753_G6 or SEAL4_753_G7 ASOs lead to statistically significant reduced expression of COL5A2 (FIG. 15M) and COL11A1 (FIG. 15N). Transfection of SEAL4_753_G6 or SEAL4_753_G7 also resulted in decreased expression of COL12A1 (FIG. 15O), FAP (FIG. 15P) and FN1 (FIG. 15Q).

### Example 7: Characterization of SEAL9 in TGFβ Induced CAFs

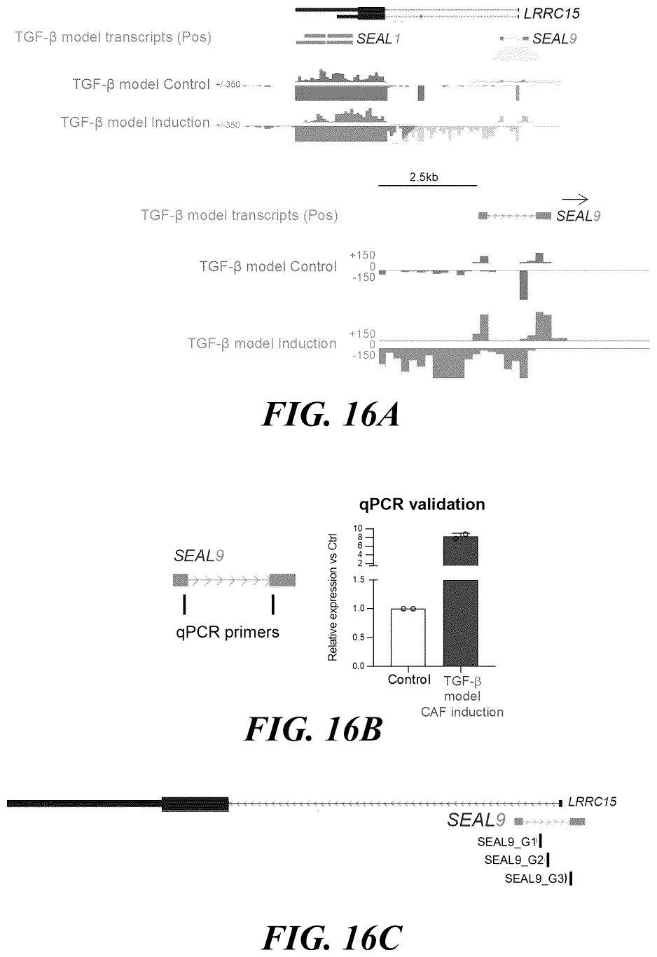

Using the same procedure as described in Example 3, the genomic locus of SEAL9 was determined. As shown in FIG. 16A, besides SEAL1 which maps to hg38 genomic coordinates chr3:194355288-194358966, the genomic locus is shared with another of the novel lncRNAs SEAL9, as well as with the protein-coding gene LRRC15, recognized as a CAF-specific marker proposed to be important for CAF-mediated pro-tumorigenic functions, immunotherapy response and patient prognosis. The LRRC15 mRNA is transcribed from the negative (−) strand and the lncRNAs SEAL1 and SEAL9 from the positive (+) strand. SEAL1 overlaps a region at the 3′ end of LRRC15, including a part of LRRC15 exon 2. SEAL9 overlaps a region at the 5′ start of LRRC15, including LRRC15 exon 1 and the annotated transcription start site (TSS), and further extends upstream of LRRC15. Both SEAL1 and SEAL9 lncRNAs were predicted to contain two exons and of non-coding potential.

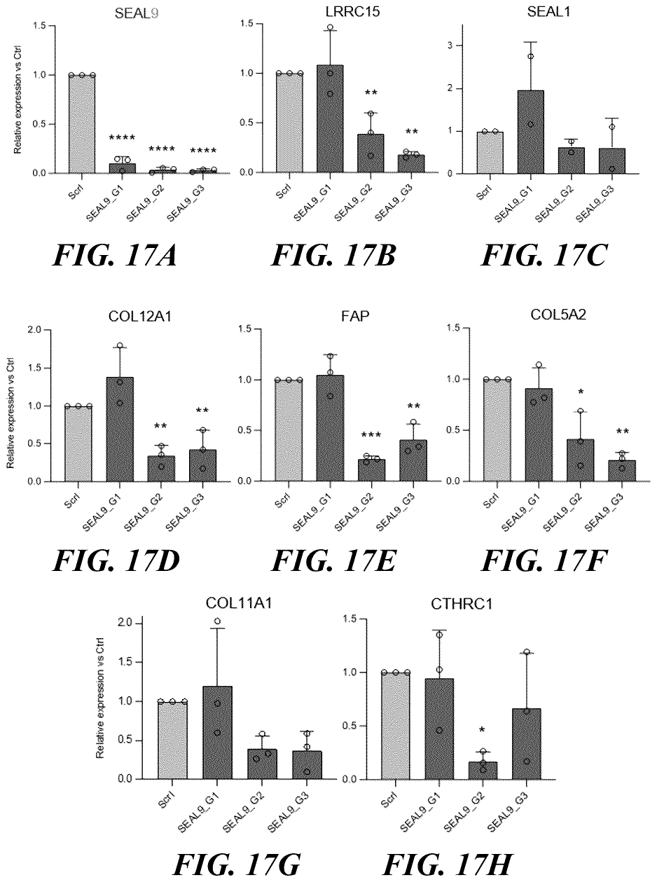

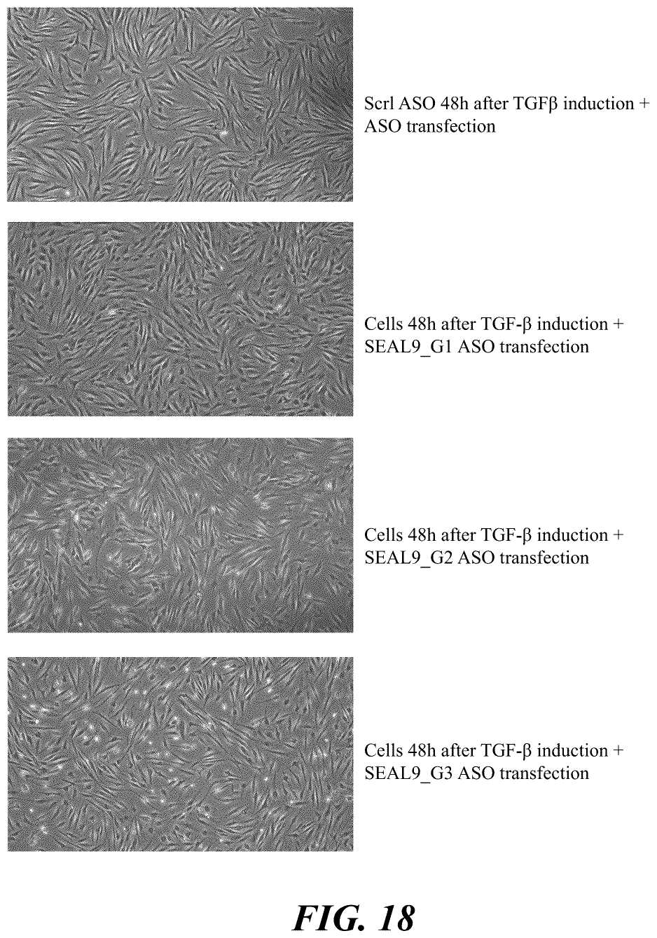

SEAL9 was upregulated in TGFβ induced CAFs compared to control. To validate the expression SEAL9 in TGFβ induced CAFs, SEAL9-specific primers were designed and qPCR performed, detecting the SEAL9 transcript in experimental replicates, as shown in FIG. 16B. SEAL9 ASOs were also designed (Table 2), as shown in FIG. 16C, to modulate SEAL9 expression in TGFβ induced CAFs. Control HDFs and TGFβ induced CAFs were transfected with Scrl (control) or SEAL9-targeting ASOs as described in Example 3, 4, 5 and 6 and evaluated after 48h for the expression of SEAL9, SEAL1, and myCAF markers (LRRC15, COL12A1, FAP, COL5A2, FOL11A1, and CTHRC1) by qPCR, as shown in FIG. 17A-17H. qPCR analysis, performed as described in Example 2, showed that transfection of SEAL9_G1, SEAL9_G2 and SEAL9_G3 ASOs clearly reduced expression of SEAL9 compared to Scrl (FIG. 17A).Transfection of SEAL9_G2 ASO resulted in statistically significant reduced expression of LRRC15 (FIG. 17B), COL12A1 (FIG. 17D), FAP (FIG. 17E), COL5A2 (FIG. 17F) and CTHRC1 (FIG. 17H). Similarly, transfection of SEAL9_G3 ASO resulted in statistically significant reduced expression of LRRC15 (FIG. 17B), COL12A1 (FIG. 17D), FAP (FIG. 17E) and COL5A2 (FIG. 17F). Moreover, COL11A1 was also reduced upon transfection with SEAL9_G2 and SEAL9_G3 ASOs (FIG. 17G). These data showed that downregulation of SEAL9 expression by ASOs reduces the expression of myCAF markers, suggesting the potential involvement of SEAL9 in CAF pro-tumorigenic function. Transfection of SEAL9_G2 or SEAL9_G3 ASOs also resulted in reduced expression of SEAL1 (FIG. 17C), implying a co-regulatory mechanism. As transfection of SEAL9_G2 and SEAL9_G3 ASOs resulted in the most decreased expression of various myCAF markers, whether these ASOs affect cell viability was next investigated. As shown in FIG. 18, microscopic images were taken of HDFs after 48 hours of TGFβ induction and SEAL9 ASO transfection or Scrl. Transfection of Scrl, SEAL9_G1, SEAL9_G2 or SEAL9_G3 ASOs resulted in high RNA yield and low toxicity. Taken together, these data suggested that SEAL9_G2 and SEAL9_G3 ASOs have high efficiency and relatively low toxicity.

### Example 8: Characterization of the Mouse Seal1 Functional Equivalent (mSeal1) in TGF-Induced Mouse Dermal Fibroblasts (MDFs)

To characterize the function of the mouse mSeal1 functional equivalent, an in vitro model analogous to the HDF model described in Example 2, was established. Immortalized MDFs (iMDFs) were cultured at 70-90% in FGM-3 Fibroblast Growth Medium-3 BulletKit (Lonza) and passaged 1-2 times/week using TrypLE Express (Gibco). For TGFβ and TGFβ+ starvation (COMB) induction, 100,000 MDFs were seeded in 6-well plates and were left to acclimate for 48 hours before induction. For TGFβ induction, cells were treated with 5 ng/ml TGFβ for 48 hours and then harvested for downstream analyses. For COMB induction, cells were treated with 5 ng/ml TGFβ in cell medium without fetal bovine serum (FBS) (starvation) for 48 hours before harvest. RNA extraction, sequencing and differential gene expression analysis was performed as described in Example 2. RNA-seq data processing was performed as described in Example 3 and visualized in the UCSC Genome Browser on GRCm39/mm39. As shown in FIG. 20A-20B, gene expression of control and TGFβ-induced iMDFs was investigated by RNA-seq. As shown in FIG. 20A, mSeal1 is located on Chr16:30088120-30093787 on the sense strand, overlapping with some region of mLrrc15 mRNA, transcribed in the opposite (anti-sense) orientation. Sequence alignment analysis, removing smaller matches of <10 nucleotides, identified 16 conserved regions between human and mouse at varying level of similarity, >90%, >75% or >50% (FIG. 20A). Expression of mSeal1 and mLrrc15 was upregulated in TGFβ induced CAF-like iMDFs compared to control (FIG. 20B). To modulate mSeal1 expression in TGFβ induced CAF-like iMDFs and investigate the effect of mSeal1 knockdown, various ASOs were designed (Table 15) to target different regions of the mSeal1 transcript as shown in FIG. 20C. The effect of targeting different regions of mSeal1 (ASOs mSeal1-6) was compared to that of targeting mLrrc15, achieved by designing ASOs targeting different regions of mLrrc15: ASOs mLrrc15_G1 (sequence 5′ TCCTATCGTCAACCGG), mLrrc15_G2 (sequence 5′-GGTGTATTAGTCGTCC-3′) and mLrrc15_G3 (sequence 5′-GTAAACGCTTCCGATG-3′) (FIG. 20C). Next, iMDFs were induced by TGFβ at the same time as transfection with ASOs. Briefly, 100,000 iMDFs per well of 6-well plate were plated one day before transfection with mSeal1, mLrrc15 or Scrl control ASOs. Right before ASO transfection, the cell medium was changed to medium containing 5 ng/ml TGFβ. ASOs were diluted in TE buffer to a working solution of 20 M and was mixed with OptiMEM medium and X-tremeGENE HP transfection reagent. The mixture was incubated at room temperature for minimum of 15 minutes, and then added dropwise to the cells at 100 l/well, with the total amount of 100 pmol ASO/well. Cells were harvested after 48 hours for RNA extraction and RNA-seq analysis. As shown in FIG. 20D, iMDFs with TGFβ induction and ASO (Scrl, mSeal1_G5, mSeal1_G6 or mLrrc15_G3 ASOs) transfection were still viable and healthy, as compared to control iMDFs. An acceptable toxic effect was observed with the mSeal1_G6 ASO (FIG. 20D, bottom). As shown in FIG. 20E-20F, bulk RNA-seq analysis was performed to investigate the expression of mSeal1, mLrrc15, and myCAF signature genes upon transfection with mSeal1_G6 (mSeal1_6) or mLrrc15_G3 (mLrrc15_3) ASOs versus Scrl. RNA-seq analysis showed that the mSeal1_G6 (mSeal1_6) ASO reduced the mSeal1 expression in one of the two experimental replicates (FIG. 20E, left), and that the mLrrc15_G3 (mLrrc15_3) ASO reduced the mLrrc15 expression (FIG. 20E, right). Singscore calculation was performed of the mouse orthologous myCAF signature (Table 13) (FIG. 20F, left) and the mouse orthologous SEAL1 TEP signature (Table 14) (FIG. 20F, right). This analysis showed that Singscores of both signatures were reduced, with the orthologous SEAL1 TEP signature significantly reduced, upon transfection with the mSeal1_G6 (mSeal1_6) ASO, while transfection with the mLrrc15_G3 (mLrrc15_3) ASO only caused a minor reduction in the signature scores (FIG. 20F). These results suggest that the mouse mSeal1 exhibits a function in MDFs as observed in human HDFs, with mSeal1 contributing more to the myCAF identity than mLrrc15. To investigate mSeal1 expression in a mouse model of tumor development, a scRNA-seq dataset of a pancreatic ductal adenocarcinoma (PDAC) mouse model was used (Krishnamurty et al, LRRC15+ myofibroblasts dictate the stromal setpoint to suppress tumour immunity, Nature 611(7934), 2022). Raw data was obtained and analyzed as in Example 1 to annotate cell types, identify FBs, sub-cluster FBs and quantify mSeal1 and mLrrc15. FIG. 20G shows a description of the PDAC mouse model. Mouse PDAC tumor cells were implanted into DTR− or DTR+ genotype mice, and initiated PDAC tumor development. DTR− genotype mice had a wild-type (wt) mSeal1/mLrrc15 genomic locus while DTR+ genotype mice had a heterogeneously modified (DTR-GFP cassette knock-in) mSeal1/mLrrc15 genomic locus, whereby both mSeal1 and mLrrc15 coding sequences were disrupted (figure modified from Krishnamurty et al, Nature 2022). In DTR+ genotype mice, diphtheria toxin (DT) is produced from the modified mSeal1/mLrrc15 genomic locus in cells where the mLrrc15 promoter is active, leading to depletion of these cells upon treating the DTR+ mice with DT. Thus, this model captures a mSeal1/mLrrc15 locus depletion phenotype. FIG. 20H-20I shows the scRNA-seq analysis of the mSeal1/mLrrc15 locus depletion phenotype. Seven fibroblasts sub-clusters were identified, with fibroblast sub-clusters 2 and 4 present in pre-implant (naïve) skin (FIG. 20H, top left and middle). 10 days after tumor implant (Day 0 sample), the fibroblast sub-clusters 0, 1, 3, 5, and 6 appeared with sub-clusters 0, 1, and 3 having the most fibroblasts and representing CAF activation (FIG. 20H, top right). During tumor development (Day 14 DTR− sample), the sub-clusters 0, 1, and 3 became more dominant in DTR− mice, the myCAFs with an active Lrrc15 promoter present (FIG. 20H, bottom left.) In DTR+ mice with DT administration (Day 14 DTR+ sample), the sub-cluster 0 was clearly depleted confirming its identity as mLrrc15/mSeal1-expressing myCAFs (FIG. 20H, bottom middle). The depletion of sub-cluster 0 was accompanied with an increase in sub-cluster 1 and 3 fibroblasts (FIG. 20H, bottom middle). Upon removal of DT, the sub-cluster 0 re-emerged in DTR+ mice (Day 21 sample), further confirming the identity (FIG. 20H, bottom right). FIG. 20I (top panel) showed that mSeal1 was absent in pre-implant fibroblasts, while specifically induced in Day 14 DTR− sample sub-cluster 0 fibroblasts and completely depleted in Day 14 DTR+ sample sub-cluster 0 fibroblasts. Sub-clusters 1 and 3 that were increased in Day 14 DTR+ samples did not display mSeal1 expression (FIG. 20I, top middle). mSeal1 also re-emerged in Day 21 DTR+ sample sub-cluster 0 fibroblasts upon removal of DT (FIG. 20I, top right). mLrrc15 also showed the strongest expression in Day 14 DTR− sample sub-cluster 0 fibroblasts (FIG. 20I, bottom middle) while being mostly absent in pre-implant fibroblasts (FIG. 20I, bottom left), and completely depleted in Day 14 DTR+ sample sub-cluster 0 fibroblasts (FIG. 20I, bottom middle). While sub-clusters 1 and 3 fibroblasts that were increased in Day 14 DTR+ samples did not display mSeal1 expression, some mLrrc15 expression was detected in those cells (FIG. 20I, bottom middle), suggesting a slightly different expression pattern than mSeal1. Upon DT removal, mLrrc15 expression re-emerged in Day 21 DTR+ sample sub-cluster 0 fibroblasts, as well as retained in sub-cluster 1 and 3 fibroblasts. These data suggest that mSeal1 may be induced during PDAC CAF activation and tumor progression. Consistently, as shown in FIGS. 4l and 4m from Krishnamurty et al, both tumor weights and volumes in DTR+ mice were reduced compared to DTR− mice with or without immunotherapy (anti-PDL1) treatment, and the reduction was further potentiated by anti-PDL1 treatment in DTR+ mice. These data independently prove that DTR+ mice were more susceptible to immunotherapy treatment (anti-PDL1), than DTR− mice. These results also suggested that an intact mSeal1/mLrrc15 locus, and expression of mSeal1 and mLrrc15, confers resistance to immunotherapy, strengthening the idea that therapeutic targeting of mSeal1 may be of clinical benefit. The expression of the murine mSeal1 was further confirmed in a scRNA-seq dataset (Samstein et al, Mutations in BRCA1 and BRCA2 differentially affect the tumor microenvironment and response to checkpoint blockade immunotherapy. Nature Cancer 1(12), 2021) from 8 tumor samples of a mouse model of TNBC, the 4T1 syngeneic mouse model. mSeal1 was expressed in the same sub-population of fibroblasts as mLrrc15, and that displayed expression of mouse disease-state fibroblast gene expression (signature defined by Buechler et al, Cross-tissue organization of the fibroblast lineage. Nature 593(7860), 2021).

TABLE 15
Sequences of exemplary ASO candidates
targeting murine Seal1 (mSeal1)
IncRNA (chr16:30088120-30093787)

| SEQ | | | |
|---|---|---|---|
| ID | | | |
| NO. | ASO name | Sequence | Target |
| 2065 | mSeal1_G1/ | GGACGACTAATACACC | mSeal1 |
| | mSeal_1 | | |
| 2066 | mSeal1_G2/ | AGTCTCCGCAAGTAGT | mSeal1 |
| | mSeal_2 | | |
| 2067 | mSeal1_G3/ | TAGATGCGGCTTGTAA | mSeal1 |
| | mSeal_3 | | |
| 2068 | mSeal1_G4/ | GCTTGACACAGTACCG | mSeal1 |
| | mSeal_4 | | |
| 2069 | mSeal1_G5/ | CTCGCAGTACAGTTAC | mSeal1 |
| | mSeal_5 | | |
| 2070 | mSeal1_G6/ | TGTGCCACGGCTTGAC | mSeal1 |
| | mSeal_6 | | |

While preferred instances of the present disclosure have been shown and described herein, it will be obvious to those skilled in the art that such instances are provided by way of example only. It is not intended that the disclosure be limited by the specific examples provided within the specification. While the disclosure has been described with reference to the aforementioned specification, the descriptions and illustrations of the instances herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the instances of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed is:

1. A modulator of a long non-coding RNA (lncRNA), wherein an expression or activity of the lncRNA is associated with an activation of a cancer-associated fibroblast (CAF).

2. The modulator of claim 1, wherein the expression or activity of the lncRNA is upregulated in the CAF compared to a fibroblast not associated with cancer.

3. The modulator of claim 1 or 2, wherein the CAF exhibits a higher expression of an ECM-modulatory gene when compared to a fibroblast that is not associated with cancer.

4. The modulator of claim 3, wherein the ECM-modulatory gene is selected from the group consisting of C1QTNF3, COL5A2, ITGA11, PDPN, POSTN, ACTA2, ACTN1, ADAM12, ADAMTS12, AEBP1, ALDH18A1, ANTXR1, ARF4, ARL4C, BACE2, BASP1, BGN, BHLHE40, BMP1, BST2, C11orf24, C1orf198, C1QTNF6, CADM1, CALD1, CALU, CCND1, CD276, CDC42EP3, CERCAM, CHN1, CHPF, CKAP4, CLEC11A, CLIC4, CNN2, COL10A1, COL11A1, COL12A1, COL1A1, COL1A2, COL5A1, COL8A1, COLGALT1, CREB3L1, CSRP2, CTHRC1, CTSB, CTSK, CTSZ, CXCL2, CXCL3, DAP, DIO2, DPYSL3, DUSP10, EDIL3, EDNRA, EFEMP2, EGFL6, ERN1, FAP, FBXO32, FKBP10, FN1, FSCN1, FZD1, GAPDH, GEM, GGT5, GJA1, GLT8D2, GOLM1, GPX7, GPX8, GREM1, HAPLN3, HCFC1R1, HES4, HLA-B, HLA-C, HS3ST3A1, ID1, ID4, IER3, IFI27, IFI6, IL32, INHBA, ITGB5, ITPRIP, KDELR2, KDELR3, KIAA1217, KIF26B, KLF6, LAMP5, LEF1, LMCD1, LMO7, LOXL2, LRRC15, LUM, MAGED1, MARCKSL1, MARVELD1, MDK, MICAL2, MIF, MMP11, MMP14, MMP19, MMP2, MSRB3, MXRA5, MYH9, MYL9, NEK6, NR4A2, NREP, NRP2, NTM, NXN, OLFML2B, P3H1, P3H4, P4HA3, P4HB, PALLD, PARVA, PDGFC, PDLIM7, PEA15, PERP, PKM, PLOD1, PLOD2, PMAIP1, PMEPA1, PODNL1, POSTN, PRDX4, PRSS23, PTGER3, PTK7, PYCR1, RAB31, RAI14, RBM3, RCAN2, RCN1, RCN3, RGCC, RGS3, RIN2, RNF144A, ROR2, RUNX2, SCARF2, SDC1, SEC13, SERPINHI, SFRP2, SHISA5, SLC16A3, SLC38A5, SLC39A14, SMCO4, SMIM3, SMYD3, SNAI2, SPARC, SPATS2L, SPHK1, SPON1, SSR3, STK17B, SUGCT, SULF1, SULF2, SYTL2, TAGLN, TENM3, TGFB1I1, TGFBI, THBS2, THY1, TMEM119, TMEM263, TMEM45A, TNFAIP3, TOM1, TPM1, TPM4, TPST2, TSPO, TUBA1C, TUSC3, UBTD1, UNC5B, VCAN, VGLL4, and VOPPl.

5. The modulator of any one of the preceding claims, wherein one or more enhancers are within a genomic locus encoding the lncRNA, wherein the one or more enhancers are associated with the CAF.

6. The modulator of any one of the preceding claims, wherein the expression or activity of the lncRNA is not upregulated in a cell that is not associated with cancer.

7. The modulator of any one of the preceding claims, wherein one or more protein-coding genes are at most 100, 200, 300, 400, or 500 nucleotides upstream or downstream of or within the genomic locus encoding the lncRNA, wherein the one or more protein-coding genes are associated with the CAF.

8. The modulator of claim 7, the one or more protein-coding genes are associated with a pro-tumorigenic or fibrosis development function of the CAF.

9. The modulator of claim 7 or 8, the one or more protein-coding genes are selected from the group consisting of LRRC15, COL12A1, DYRK2, FN1, and CAPN9.

10. The modulator of any one of the preceding claims, wherein the lncRNA comprises any one of lncRNAs listed in Table 10 or Table 12, or a fragment thereof.

11. The modulator of any one of the preceding claims, wherein the lncRNA comprises XLOC 055514, XLOC_055515, XLOC_069921, XLOC 005184, ENSG00000203585, SHARED_00113753, ENSG00000288903, ENSG00000230838 (LINC01614), ENSG00000244137, or a fragment thereof, as listed in Table 10.

12. The modulator of any one of the preceding claims, wherein the modulator comprises an endonuclease complex guided by a nucleic acid, wherein the nucleic acid targets the lncRNA or a genomic locus thereof.

13. The modulator of claim 12, wherein the modulator is an ASO-directed RNA editing complex, a CRISPR-directed DNA editing complex, or a CRISPR-directed RNA editing complex.

14. The modulator of any one of claims 1 to 13, wherein the modulator comprises a nucleic acid molecule that hybridizes to the lncRNA.

15. The modulator of claim 14, wherein the nucleic acid molecule is a small interfering RNA (siRNA), a microRNA (miRNA), an inhibitory double stranded RNA (dsRNA), a small or short hairpin RNA (shRNA), an antisense oligonucleotide (ASO), a piwi-interacting RNA (piRNA), a heterogeneous nuclear RNA (hnRNA), a small nuclear RNA (snRNA), or an enzymatically-prepared siRNA (esiRNA) or the precursors thereof.

16. The modulator of claim 15, wherein the ASO is a gapmer or a mixmer.

17. The modulator of claim 16, wherein the ASO is about 13-30, or about 14-18 nucleotides long.

18. The modulator of any one of claims 15-17, wherein the nucleic acid molecule comprises at least 10, 11, 12, or 13 consecutive nucleotides with no more than 1, 2, or 3 mismatches from one of SEQ ID NOs: 1-40.

19. The modulator of any one of claims 15-18, wherein the nucleic acid molecule comprises a nucleic acid sequence that is at least 80%, at least 85%, at least 90%, at least 95% identical to a sequence selected from one of SEQ ID NOs: 1-40.

20. The modulator of any one of claims 15-19, wherein the ASO comprises 5′-wing region and 3′-wing region, and at least 5′-wing region and 3′-wing region comprises a nucleic acid analogue selected from a 2′-methoxyethyl (2′-MOE) RNA and a locked nucleic acid (LNA).

21. The modulator of claim 20, wherein the LNA comprises a beta-D-oxy LNA, an alpha-L-oxy-LNA, a beta-D-amino-LNA, an alpha-L-amino-LNA, a beta-D-thio-LNA, an alpha-L-thio-LNA, a 5′-methyl-LNA, a beta-D-ENA, or an alpha-L-ENA.

22. The modulator of any one of claims 20 and 21, wherein the 5′-wing region comprises at least two LNAs.

23. The modulator of any one of claims 20-22, wherein the 5′-wing region comprises three consecutive LNAs.

24. The modulator of any one of claims 20-23, wherein the 3′-wing region comprises at least one LNA.

25. The modulator of any one of claims 20-24, wherein the 3′-wing region comprises two consecutive LNAs.

26. The modulator of any one of claims 15-25, wherein the ASO comprises one or more phosphorothioate internucleotide linkages.

27. The modulator of any one of claims 15-26, wherein each internucleotide linkage is a phosphorothioate backbone.

28. A modulator comprising an antisense oligonucleotide (ASO), wherein the ASO comprises at least 10, 11, 12, or 13 consecutive nucleotides with no more than 1, 2, or 3 mismatches from SEQ ID NO: 2, 4, 5, 9-11, 15, 16, 18, 19, 24-26, 34 or 38-40.

29. The modulator of claim 28, wherein the ASO comprises a nucleic acid sequence of 80%, at least 85%, at least 90%, at least 95% identical to SEQ ID NO: 2, 4, 5, 9-11, 15, 16, 18, 19, 24-26, 34 or 38-40.

30. A pharmaceutical composition comprising the modulator of any one of claims 1-29 and a pharmaceutically acceptable salt or derivative thereof.

31. A kit comprising the modulator of any one of claims 1 to 29 or the pharmaceutical composition of claim 30.

32. A method of inhibiting a growth of a solid tumor or facilitating access to a solid tumor in a subject in need thereof, the method comprising administering to the subject an effective amount of the modulator of any one of claims 1 to 29 or the pharmaceutical composition of claim 30.

33. The method of claim 32, wherein the modulator of any one of claims 1 to 29 or the pharmaceutical composition of claim 30 reduces expression or activity of genes involved in ECM structure organization, and/or regulation of GTPase activity, and/or regulation of tumor innervation in a tumor microenvironment.

34. The method of claim 33, wherein the genes involved in ECM structure organization and/or regulation of GTPase activity and/or regulation of tumor innervation in a tumor microenvironment is selected from the group consisting of LRRC15, MMP11, COL11A1, C1QTNF3, CTHRCT, COLT2AT, COL10A1, COL5A2, THBS2, AEBPT, ITGATT, PDPN, and FAP.

35. The method of claim 32, wherein the modulator of any one of claims 1 to 29 or the pharmaceutical composition of claim 30 reduces an expression or activity of one or more genes shown in Table 1, Table 13, or Table 14.

36. The method of any one of claims 32 to 35, wherein the modulator of any one of claims 1-29 is encapsulated in a liposome or coupled with a nanoparticle.

37. The method of any one of claims 32 to 36, wherein the modulator of any one of claims 1-29 is administered in combination with an anti-tumor drug.

38. A method of diagnosing or monitoring a cancer in a subject, the method comprising: (a) obtaining a sample from the subject; (b) detecting an expression and/or an activity of a biomarker from the sample, wherein the biomarker comprises an lncRNA comprising a nucleic acid sequence listed in Table 11 or a fragment thereof, or transcribed from a genomic region or a subset thereof listed in Table 10 or Table 12; and (c) diagnosing or monitoring the cancer prognosis based on the expression and/or the activity of the biomarker.

39. A method of predicting severity and progression of a cancer in a subject, the method comprising: (a) obtaining a sample from the subject; (b) detecting an expression and/or an activity of a biomarker from the sample, wherein the biomarker comprises an lncRNA comprising a nucleic acid sequence listed in Table 11 or a fragment thereof, or transcribed from a genomic region or a subset thereof listed in Table 10 or Table 12; and (c) diagnosing the subject to have a more severe or a progression of the cancer if the expression and/or the activity of the biomarker is at least 50%, at least 60%, at least 70%, at least 80%, or at least 90% higher when compared to a control.

40. A method of monitoring an efficacy or therapeutic resistance of a therapy treating a cancer in a subject, the method comprising: (a) obtaining a sample from the subject; (b) detecting an expression and/or an activity of a biomarker from the sample, wherein the biomarker comprises an lncRNA comprising a nucleic acid sequence listed in Table 11 or a fragment thereof, or transcribed from a genomic region or a subset thereof listed in Table 10 or Table 12; and (c) concluding the therapy treating the cancer is effective or is less likely to develop therapeutic resistance if the expression and/or the activity of the biomarker is at least 50%, at least 60%, at least 70%, at least 80%, at least 90% reduced by the therapy.

41. The method of any one of claims 38-40, wherein the biomarker further comprises INHBA, COL11A1, MMP13, NNMT, LOXL3, PLOD2, TNC, ITGA11, COL5A1, COMP, COL1A1, FN1, LTBP2, COL10A1, NOX4, POSTN, CCN2, LRRC15, DCN, LUM, COL1A2, COL3A1, COL6A2, MMP11, COL11A1, C1QTNF3, CTHRC1, COL12A1, COL10A1, COL5A2, THBS2, AEBP1, ITGA11, PDPN, FAP, COL8A1, or a gene from Table 1, Table 13, or Table 14.

42. The method of any one of claims 38-41, wherein the method further comprises (d) administering the subject the modulator of any one of claims 1-29, or the pharmaceutical composition of claim 30.

43. The method of any one of claims 38-42, wherein the cancer is a solid tumor.

44. The method of claim 43, wherein the solid tumor is breast cancer, colorectum cancer, lung cancer, prostate cancer, bladder cancer, head and neck cancer (e.g., head and neck squamous cell carcinoma), skin cancer (e.g., skin melanoma), pancreatic cancer (e.g., pancreatic ductal adenocarcinoma), liver cancer, esophageal cancer, brain cancer, stomach cancer (e.g., stomach adenocarcinoma), gallbladder cancer, ovarian cancer, colon adenocarcinoma, or sarcoma.
